(19) United States
(12) Patent Application Publication
(10) Pub. No.: US 2024/0390520 A1
(43) Pub. Date: Nov. 28, 2024

(54) GENE EDITING OF PCSK9 OR ANGPTL3 AND COMPOSITIONS AND METHODS OF USING SAME FOR TREATMENT OF DISEASE

(71) Applicant: Verve Therapeutics, Inc., Boston, MA (US)

(72) Inventors: Andrew M. Bellinger, Wellesley, MA (US); Kallanthottathil G. Rajeev, Wayland, MA (US); Caroline Reiss, Somerville, MA (US); Jamie Denizio, Boston, MA (US); Hariharan Jayaram, Cambridge, MA (US); Sowmya Iyer, Boston, MA (US); Sara Cristina De Almeida Pinto Garcia, Brookline, MA (US); Kui Wang, Boston, MA (US); Alexandra Chadwick, Somerville, MA (US); Christopher Cheng, North Reading, MA (US); Richard Glenn Lee, Carlsbad, CA (US); Ellen Rohde, Ashland, MA (US)

(21) Appl. No.: 18/611,988

(22) Filed: Mar. 21, 2024

Related U.S. Application Data
(63) Continuation of application No. PCT/US2022/044453, filed on Sep. 22, 2022.
(60) Provisional application No. 63/247,236, filed on Sep. 22, 2021.
(60) Provisional application No. 63/255,333, filed on Oct. 13, 2021.
(60) Provisional application No. 63/389,679, filed on Jul. 15, 2022.

(51) Int. Cl. A61K 48/00; A61P 9/10; C12N 9/22; C12N 9/80; C12N 15/11

(52) U.S. Cl. CPC A61K 48/005; A61P 9/10; C12N 9/22; C12N 9/80; C12N 15/111; C12N 2310/20; C12N 2310/321

(57) ABSTRACT

Disclosed herein are novel gene editing systems capable of being delivered to a subject intravenously through a lipid nano particle pharmaceutical formulation and producing durable in vivo editing of a target gene, such as ANGPTL3, with high on-target gene editing efficiency, reduced or low off-target effect, and no germline editing. The gene editing systems comprise a chemically modified guide nucleic acid sequence with a spacer having a specified arrangement of deoxyribonucleotides and ribonucleotides. The novel gene editing systems comprise mRNA that encodes the gene editor proteins, which may include a modified nickase component. Methods of disease treatments using the gene editing systems are also disclosed.

### CROSS REFERENCE TO RELATED APPLICATIONS

This application is a continuation of International Application No. PCT/US2022/044453, filed Sep. 22, 2022 which claims benefit under 35 U.S.C. § 119 from Provisional Application Ser. No. 63/247,236, filed Sep. 22, 2021; Provisional Application Ser. No. 63/255,333, filed Oct. 13, 2021; and Provisional Application Ser. No. 63/389,679, filed Jul. 15, 2022, the disclosures of which each are incorporated herein by reference in their entirety.

### SEQUENCE LISTING

The instant application contains a Sequence Listing which has been submitted electronically in XML format and is hereby incorporated by reference by its entirety. Said XML copy, created Aug. 2, 2024, is named 53989-715.301 Replacement_SL_.xml and is 6,731,884 bytes in size.

### SUMMARY OF THE INVENTION

Described herein is an in vivo hybrid guide gene editing system comprising: (a) a gene editor protein or a component thereof comprising a nucleic acid binding domain, or a nucleic acid encoding the gene editor protein or the component thereof; and (b) a hybrid guide nucleic acid comprising (i) a spacer sequence comprising a deoxyribonucleotide and a ribonucleotide comprising a ribose, wherein a 2′ hydroxyl group of the ribose is covalently linked to a methyl group (2′-OMe), and (ii) a binding scaffold for the gene editor protein or component thereof, wherein (a) and (b) are constituent components of a pharmaceutical composition that comprises a lipid nanoparticle (LNP) containing (a) or (b), wherein the LNP comprises an amino lipid, a phospholipid, a sterol and a PEG lipid. In some embodiments, the spacer sequence corresponds to a protospacer on a target gene, and wherein the target gene is ANGPTL3. In some embodiments, the gene editor protein or the component thereof comprises a deaminase.

Described herein is an in vivo hybrid guide gene editing system comprising: (a) a gene editor protein or a component thereof comprising a nucleic acid binding domain, or a nucleic acid encoding the gene editor protein or the component thereof; and (b) a hybrid guide nucleic acid comprising (i) a spacer sequence comprising a deoxyribonucleotide and a ribonucleotide, wherein the spacer sequence corresponds to a protospacer on an ANGPTL3 gene, and (ii) a binding scaffold for the gene editor protein or component thereof, wherein (a) and (b) are constituent components of a pharmaceutical composition that comprises a lipid nanoparticle (LNP) containing (a) or (b), wherein the LNP comprises an amino lipid, a phospholipid, a sterol and a PEG lipid. In some embodiments, the gene editor protein or the component thereof comprises a deaminase. In some embodiments, the ribonucleotide comprises a ribose, and wherein the ribose comprises a 2′ hydroxyl group covalently linked to a methyl group (2′-OMe).

Described herein is an in vivo hybrid guide gene editing system comprising: (a) a gene editor protein or a component thereof comprising a nucleic acid binding domain and a deaminase, or a nucleic acid encoding the gene editor protein or the component thereof; and (b) a hybrid guide nucleic acid comprising (i) a spacer sequence comprising a deoxyribonucleotide and a ribonucleotide, and (ii) a binding scaffold for the gene editor protein or component thereof, wherein (a) and (b) are constituent components of a pharmaceutical composition that comprises a lipid nanoparticle (LNP) containing (a) or (b), wherein the LNP comprises an amino lipid, a phospholipid, a sterol and a PEG lipid. In some embodiments, the spacer sequence corresponds to a protospacer on a target gene, and wherein the target gene is ANGPTL3. In some embodiments, the ribonucleotide comprises a ribose, and wherein the ribose comprises a 2′ hydroxyl group covalently linked to a methyl group (2′-OMe).

In some embodiments, the spacer sequence comprises an unmodified ribonucleotide. In some embodiments, wherein the nucleic acid encoding the gene editor protein or the component thereof is an mRNA. In some embodiments, the gene editor protein or the component thereof comprises a single fusion protein or two or more proteins. In some embodiments, the spacer sequence comprises a phosphorothioate backbone modification (PS). In some embodiments, the spacer sequence comprises a (2′-OMe)PS(2′-OMe)PS(DNA)PS(DNA)(RNA)(DNA)(DNA)(DNA) motif. In some embodiments, the (2′-OMe)PS(2′-OMe)PS(DNA)PS(DNA)(RNA)(DNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 30 or 31. In some embodiments, the spacer sequence comprises a (2′-OMe)PS(2′-OMe)PS(DNA)PS(DNA)(RNA)(DNA)(DNA) motif. In some embodiments, the (2′-OMe)PS(2′-OMe)PS(DNA)PS(DNA)(RNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 28 or 29. In some embodiments, the spacer sequence comprises a (2′-OMe)PS(2′-OMe)PS(DNA)PS(DNA)(DNA)(DNA)(DNA) motif. In some embodiments, the (2′-OMe)PS(2′-OMe)PS(DNA)PS(DNA)(DNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 5, 11 or 12. In some embodiments, the hybrid guide nucleic acid comprises a sequence with at least about 80%, about 90%, about 95%, about 99%, about 99.5%, or about 100% sequence identity or sequence similarity of a sequence selected from the group consisting of SEQ ID Nos: 110-113. In some embodiments, the hybrid guide nucleic acid comprises a sequence with at least about 80%, about 90%, about 95%, about 96%, about 97%, about 98%, about 99%, about 99.5%, or about 100% sequence identity or sequence similarity of a sequence selected from the group consisting of SEQ ID Nos: 106-109. In some embodiments, the guide nucleic acid comprises a sequence with at least about 80%, about 90%, about 95%, about 96%, about 97%, about 98%, about 99%, about 99.5%, or about 100% sequence identity or sequence similarity of a sequence selected from the group consisting of SEQ ID Nos: 79-82.

In some embodiments, the nucleic acid binding domain is capable of binding to DNA. In some embodiments, the nucleic acid binding domain is capable of binding to RNA. In some embodiments, the deoxyribonucleotide is located on position 3, 4, 6, 7 or 8 from the 5′ end of the spacer sequence. In some embodiments, the spacer sequence comprises deoxyribonucleotides on positions 3, 4, 6 and 7 from the 5′ end of the spacer sequence. In some embodiments, the spacer sequence comprises deoxyribonucleotides on positions 3 and 4 from the 5′ end of the spacer sequence. In some embodiments, the spacer sequence comprises deoxyribonucleotides on positions 6 and 7 from the 5′ end of the spacer sequence. In some embodiments, the spacer sequence comprises deoxyribonucleotides on positions 3, 4, 6, 7 and 8 from the 5′ end of the spacer sequence. In some embodiments, the spacer sequence comprises one to ten deoxyribonucleotides. In some embodiments, the spacer sequence comprises one to five deoxyribonucleotides. In some embodiments, the DNA binding domain comprises a CRISPR protein or a fragment thereof. In some embodiments, the DNA binding domain comprises a catalytically impaired nuclease. In some embodiments, the DNA binding domain comprises a prime editing protein or a fragment thereof.

In some embodiments, the gene editor protein or the component thereof affects less than about 10%, about 9%, about 8%, about 7%, about 6%, about 5%, about 4%, about 3%, about 2%, or about 1% editing on all off-target sites as compared to a gene editing system comprising a corresponding gRNA without the deoxyribonucleotide. In some embodiments, the gene editor protein or the component thereof affects over about 50%, about 60%, about 70%, about 80%, about 90%, or about 95% editing on the target gene as compared to a gene editing system comprising a corresponding gRNA without the deoxyribonucleotide. In some embodiments, the gene editor protein or the component thereof affects over about 95%, about 96%, about 97%, about 98%, or about 99% editing on the target gene as compared to a gene editing system comprising a corresponding gRNA without the deoxyribonucleotide. In some embodiments, the gene editor protein or the component thereof affects from about 95% to about 99% editing on the target gene as compared to a gene editing system comprising a corresponding gRNA without the deoxyribonucleotide. In some embodiments, the LNP comprises a N-acetylgalactosamine (GalNAc) lipid receptor targeting conjugate. In some embodiments, the target gene is expressed in liver, or cells or tissues of liver origin. In some embodiments, the target gene is expressed in a non-liver organ or cells or tissues of non-liver origin.

Described herein is a method for treating or preventing an atherosclerotic cardiovascular disease in a subject in need thereof, the method comprising administering to the subject a therapeutically effective amount of the in vivo hybrid guide gene editing system described herein. In some embodiments, the subject is a primate. In some embodiments, the primate is human.

Described herein is a gene editing system comprising: (a) a gene editor protein or a component thereof comprising a nucleic acid binding domain, or a nucleic acid encoding the gene editor protein or the component thereof; and (b) a hybrid guide nucleic acid comprising a spacer sequence, wherein the spacer sequence comprises a deoxyribonucleotide and a ribonucleotide comprising a ribose, and wherein a 2′ hydroxyl group of the ribose is covalently linked to a methyl group (2′-OMe).

Described herein is a gene editing system comprising: (a) a gene editor protein or a component thereof comprising a nucleic acid binding domain, or a nucleic acid encoding the gene editor protein or the component thereof; and (b) a hybrid guide nucleic acid comprising a spacer sequence, wherein the spacer sequence comprises a deoxyribonucleotide and a ribonucleotide, and wherein the spacer sequence corresponds to a protospacer on an ANGPTL3 gene.

Described herein is a gene editing system comprising: (a) a gene editor protein or a component thereof comprising a nucleic acid binding domain and a deaminase, or a nucleic acid encoding the gene editor protein or the component thereof, and (b) a hybrid guide nucleic acid comprising a spacer sequence, wherein the spacer sequence comprises a deoxyribonucleotide and a ribonucleotide.

Described herein is a hybrid guide nucleic acid for a gene editing system, wherein the hybrid guide comprises (i) a spacer sequence comprising a deoxyribonucleotide and a ribonucleotide comprising a ribose, wherein a 2′ hydroxyl group of the ribose is covalently linked to a methyl group (2′-OMe), and (ii) a binding scaffold. In some embodiments, the spacer sequence comprises a (2′-OMe)PS(2′-OMe)PS(DNA)PS(DNA)(RNA)(DNA)(DNA)(DNA) motif. In some embodiments, the (2′-OMe)PS(2′-OMe)PS(DNA)PS(DNA)(RNA)(DNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 30 or 31. In some embodiments, the spacer sequence comprises a (2′-OMe)PS(2′-OMe)PS(DNA)PS(DNA)(RNA)(DNA)(DNA) motif. In some embodiments, the (2′-OMe)PS(2′-OMe)PS(DNA)PS(DNA)(RNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 28 or 29. In some embodiments, the spacer sequence comprises a (2′-OMe)PS(2′-OMe)PS(DNA)PS(DNA)(DNA)(DNA)(DNA) motif. In some embodiments, the (2′-OMe)PS(2′-OMe)PS(DNA)PS(DNA)(DNA)(DNA)(DNA) motif is located at the 5′ end of the spacer sequence. In some embodiments, the spacer sequence comprises a sequence with at least about 80%, about 90%, about 95%, about 99%, about 99.5%, or about 100% sequence identity or sequence similarity of SEQ ID NO: 5, 11 or 12. In some embodiments, the hybrid guide nucleic acid comprises a sequence with at least about 80%, about 90%, about 95%, about 99%, about 99.5%, or about 100% sequence identity or sequence similarity of a sequence selected from the group consisting of SEQ ID Nos: 110-113. In some embodiments, the hybrid guide nucleic acid comprises a sequence with at least about 80%, about 90%, about 95%, about 96%, about 97%, about 98%, about 99%, about 99.5%, or about 100% sequence identity or sequence similarity of a sequence selected from the group consisting of SEQ ID Nos: 106-109. In some embodiments, the guide nucleic acid comprises a sequence with at least about 80%, about 90%, about 95%, about 96%, about 97%, about 98%, about 99%, about 99.5%, or about 100% sequence identity or sequence similarity of a sequence selected from the group consisting of SEQ ID Nos: 79-82.

Described herein is a composition comprising a Cas9 nickase, wherein the Cas9 nickase comprises a mutation, and wherein the mutation is selected from the group consisting of: N692A, M694A, Q695A, H698A, K810A, K855A, K848A, K1003A, R1060A as compared to a Cas9 nickase of SEQ ID NO: 695 In some embodiments, the Cas9 nickase comprises a sequence with at least 90% identity to SEQ ID NO: 695. In some embodiments, the Cas9 nickase comprises a sequence with at least 95% identity to SEQ ID NO: 695. In some embodiments, the Cas9 nickase comprises a sequence with at least 98% identity to SEQ ID NO: 695. In some embodiments, the Cas9 nickase comprises a sequence with at least 99% identity to SEQ ID NO: 695. In some embodiments, the Cas9 nickase comprises a sequence with at least 99.5% identity to SEQ ID NO: 695. In some embodiments, the mutation comprises N692A, M694A, Q695A and H698A as compared to the Cas9 nickase of SEQ ID NO: 695. In some embodiments, the mutation comprises K855A as compared to the Cas9 nickase of SEQ ID NO: 695. In some embodiments, the mutation comprises K848A, K1003A and R1060A as compared to the Cas9 nickase of SEQ ID NO: 695. In some embodiments, the mutation comprises K810A, K1003A, R1060A as compared to the Cas9 nickase of SEQ ID NO: 695.

Described herein is a composition comprising a nucleic acid encoding a Cas9 nickase, wherein the nucleic acid encoding the Cas9 nickase comprises a mutation, and wherein the mutation is selected from the group consisting of: AAC at codon 692 is mutated to GCC, AUG at codon 694 is mutated to GCC, CAG at codon 695 is mutated to GCC, CAC at codon 698 is mutated to GCC, AAG at codon 855 is mutated to GCC, AAG at codon 810 is mutated to GCC, AAG at codon 848 is mutated to GCC, AAG at codon 1003 is mutated to GCC, and CGG at codon 1060 is mutated to GCC as compared to a nucleic acid of SEQ ID NO: 694. In some embodiments, the nucleic acid encoding the Cas9 nickase comprises a sequence with at least 90% identity to SEQ ID NO: 694. In some embodiments, the nucleic acid encoding the Cas9 nickase comprises a sequence with at least 95% identity to SEQ ID NO: 694. In some embodiments, the nucleic acid encoding the Cas9 nickase comprises a sequence with at least 98% identity to SEQ ID NO: 694. In some embodiments, the nucleic acid encoding the Cas9 nickase comprises a sequence with at least 99% identity to SEQ ID NO: 694. In some embodiments, the nucleic acid encoding the Cas9 nickase comprises a sequence with at least 99.5% identity to SEQ ID NO: 694. In some embodiments, the mutation comprises AAC at codon 692 is mutated to GCC, AUG at codon 694 is mutated to GCC, CAG at codon 695 is mutated to GCC and CAC at codon 698 is mutated to GCC as compared to the nucleic acid SEQ ID NO: 694. In some embodiments, the mutation comprises AAG at codon 855 is mutated to GCC as compared to the nucleic acid SEQ ID NO: 694. In some embodiments, the mutation comprises AAG at codon 878 is mutated to GCC, AAG at codon 1003 is mutated to GCC, and CGG at codon 1060 is mutated to GCC as compared to the nucleic acid SEQ ID NO: 694. In some embodiments, the mutation comprises AAG at codon 810 is mutated to GCC, AAG at codon 1003 is mutated to GCC, and CGG at codon 1060 is mutated to GCC as compared to the nucleic acid SEQ ID NO: 694.

Described herein is a gene editing system comprising: (a) a Cas9 nickase, and (b) a guide RNA comprising a spacer corresponding to protospacer sequence on a target gene and a binding scaffold for the Cas9 nickase, wherein the Cas9 nickase comprises a mutation, wherein the mutation is selected from the group consisting of: N692A, M694A, Q695A, H698A, K810A, K855A, K848A, K1003A, R1060A as compared to a Cas9 nickase of SEQ ID NO: 695. In some embodiments, the gene editing system comprises a deaminase. In some embodiments, the gene editing system comprises a polymerase. In some embodiments, the polymerase is a reverse transcriptase. In some embodiments, the target gene is PCSK9. In some embodiments, the target gene is ANGPTL3. In some embodiments, the Cas9 nickase comprises a sequence with at least 90% identity to SEQ ID NO: 695. In some embodiments, the Cas9 nickase comprises a sequence with at least 95% identity to SEQ ID NO: 695. In some embodiments, the Cas9 nickase comprises a sequence with at least 98% identity to SEQ ID NO: 695. In some embodiments, the Cas9 nickase comprises a sequence with at least 99% identity to SEQ ID NO: 695. In some embodiments, the Cas9 nickase comprises a sequence with at least 99.5% identity to SEQ ID NO: 695. In some embodiments, the mutation comprises N692A, M694A, Q695A and H698A as compared to the Cas9 nickase of SEQ ID NO: 695. In some embodiments, the mutation comprises K855A as compared to the Cas9 nickase of SEQ ID NO: 695. In some embodiments, the mutation comprises K848A, K1003A and R1060A as compared to the Cas9 nickase of SEQ ID NO: 695. In some embodiments, the mutation comprises K810A, K1003A, R1060A as compared to the Cas9 nickase of SEQ ID NO: 695.

Described herein is a gene editing system comprising: (a) a nucleic acid encoding a Cas9 nickase, and (b) a guide RNA comprising a spacer corresponding to protospacer sequence on a target gene and a binding scaffold for the Cas9 nickase, wherein the nucleic acid encoding the Cas9 nickase comprises a mutation, wherein the mutation is selected from the group consisting of: AAC at codon 692 is mutated to GCC, AUG at codon 694 is mutated to GCC, CAG at codon 695 is mutated to GCC, CAC at codon 698 is mutated to GCC, AAG at codon 855 is mutated to GCC, AAG at codon 810 is mutated to GCC, AAG at codon 848 is mutated to GCC, AAG at codon 1003 is mutated to GCC, and CGG at codon 1060 is mutated to GCC as compared to a nucleic acid of SEQ ID NO: 694. In some embodiments, the gene editing system comprises a nucleic acid encoding a deaminase. In some embodiments, the gene editing system comprises a nucleic acid encoding a polymerase. In some embodiments, the polymerase is a reverse transcriptase. In some embodiments, the target gene is PCSK9. In some embodiments, the target gene is ANGPTL3. In some embodiments, the nucleic acid encoding the Cas9 nickase comprises a sequence with at least 90% identity to SEQ ID NO: 694. In some embodiments, the nucleic acid encoding the Cas9 nickase comprises a sequence with at least 95% identity to SEQ ID NO: 694. In some embodiments, the nucleic acid encoding the Cas9 nickase comprises a sequence with at least 98% identity to SEQ ID NO: 694. In some embodiments, the nucleic acid encoding the Cas9 nickase comprises a sequence with at least 99% identity to SEQ ID NO: 694. In some embodiments, the nucleic acid encoding the Cas9 nickase comprises a sequence with at least 99.5% identity to SEQ ID NO: 694. In some embodiments, the mutation comprises AAC at codon 692 is mutated to GCC, AUG at codon 694 is mutated to GCC, CAG at codon 695 is mutated to GCC and CAC at codon 698 is mutated to GCC as compared to the nucleic acid SEQ ID NO: 694. In some embodiments, the mutation comprises AAG at codon 855 is mutated to GCC as compared to the nucleic acid SEQ ID NO: 694. In some embodiments, the mutation comprises AAG at codon 878 is mutated to GCC, AAG at codon 1003 is mutated to GCC, and CGG at codon 1060 is mutated to GCC as compared to the nucleic acid SEQ ID NO: 694. In some embodiments, the mutation comprises AAG at codon 810 is mutated to GCC, AAG at codon 1003 is mutated to GCC, and CGG at codon 1060 is mutated to GCC as compared to the nucleic acid SEQ ID NO: 694.

Described herein is a nucleic acid comprises a sequence selected from the group consisting of SEQ ID NOs: 723, 725, 727 and 729.

Described herein is a base editor protein, wherein the base editor protein comprises a sequence selected from the group consisting of SEQ ID NOs: 724, 726, 728, and 730.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein a total amount of the guide RNA and the mRNA is about 0.01 mg/kg to about 3 mg/kg. In some embodiments, the total amount of the guide RNA and the mRNA is about 0.1 mg/kg to about 3 mg/kg. In some embodiments, the total amount of the guide RNA and the mRNA is about 0.01 mg/kg to about 2 mg/kg. In some embodiments, the total amount of the guide RNA and the mRNA is about 0.1 mg/kg to about 2 mg/kg. In some embodiments, the total amount of the guide RNA and the mRNA is about 0.01 mg/kg to about 1.5 mg/kg. In some embodiments, the total amount of the guide RNA and the mRNA is about 0.1 mg/kg to about 1.5 mg/kg. In some embodiments, the total amount of the guide RNA and the mRNA is about 0.01 mg/kg to about 1.25 mg/kg. In some embodiments, the total amount of the guide RNA and the mRNA is about 0.1 mg/kg to about 1.25 mg/kg. In some embodiments, the total amount of the guide RNA and the mRNA is about 0.01 mg/kg to about 1 mg/kg. In some embodiments, the total amount of the guide RNA and the mRNA is about 0.1 mg/kg to about 1 mg/kg. In some embodiments, the total amount of the guide RNA and the mRNA is about 0.1 mg/kg. In some embodiments, the total amount of the guide RNA and the mRNA is about 0.2 mg/kg. In some embodiments, the total amount of the guide RNA and the mRNA is about 0.3 mg/kg. In some embodiments, the total amount of the guide RNA and the mRNA is about 0.4 mg/kg. In some embodiments, the total amount of the guide RNA and the mRNA is about 0.5 mg/kg. In some embodiments, the total amount of the guide RNA and the mRNA is about 0.6 mg/kg. In some embodiments, the total amount of the guide RNA and the mRNA is about 0.7 mg/kg. In some embodiments, the total amount of the guide RNA and the mRNA is about 0.8 mg/kg. In some embodiments, the total amount of the guide RNA and the mRNA is about 0.9 mg/kg. In some embodiments, the total amount of the guide RNA and the mRNA is about 1 mg/kg. In some embodiments, the total amount of the guide RNA and the mRNA is about 1 mg/kg to about 3 mg/kg. In some embodiments, the total amount of the guide RNA and the mRNA is about 1.1 mg/kg to about 3 mg/kg. In some embodiments, the total amount of the guide RNA and the mRNA is about 1.2 mg/kg to about 3 mg/kg. In some embodiments, the total amount of the guide RNA and the mRNA is about 1.3 mg/kg to about 3 mg/kg. In some embodiments, the total amount of the guide RNA and the mRNA is about 1.4 mg/kg to about 3 mg/kg. In some embodiments, the total amount of the guide RNA and the mRNA is about 1.5 mg/kg to about 3 mg/kg. In some embodiments, the total amount of the guide RNA and the mRNA is about 1.6 mg/kg to about 3 mg/kg. In some embodiments, the total amount of the guide RNA and the mRNA is about 1.7 mg/kg to about 3 mg/kg. In some embodiments, the total amount of the guide RNA and the mRNA is about 1.8 mg/kg to about 3 mg/kg. In some embodiments, the total amount of the guide RNA and the mRNA is about 1.9 mg/kg to about 3 mg/kg.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (b) a LNP comprising an amino lipid, a PEG lipid, a phospholipid and a sterol, wherein administration of the gene editing system results in a plasma Cmax of the mRNA in the human subject to be about 0.05 μg/mL to about 5 μg/mL. In some embodiments, the plasma Cmax of the mRNA in the human subject is about 0.1 μg/mL to about 5 μg/mL. In some embodiments, the plasma Cmax of the mRNA in the human subject is about 0.2 μg/mL to about 5 μg/mL. In some embodiments, the plasma Cmax of the mRNA in the human subject is about 0.5 μg/mL to about 5 μg/mL. In some embodiments, the plasma Cmax of the mRNA in the human subject is about 1 μg/mL to about 5 μg/mL. In some embodiments, the plasma Cmax of the mRNA in the human subject is about 2 μg/mL to about 5 μg/mL. In some embodiments, the plasma Cmax of the mRNA in the human subject is about 3 μg/mL to about 5 μg/mL. In some embodiments, the plasma Cmax of the mRNA in the human subject is about 4 μg/mL to about 5 μg/mL.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein administration of the gene editing system results in an AUC of the mRNA in the human subject to be about 1 μg×h/mL to about 100 μg×h/mL. In some embodiments, the AUC of the mRNA in the human subject is about 1 μg×h/mL to about 50 μg×h/mL. In some embodiments, the AUC of the mRNA in the human subject is about 1 μg×h/mL to about 20 μg×h/mL. In some embodiments, the AUC of the mRNA in the human subject is about 1 μg×h/mL to about 10 μg×h/mL. In some embodiments, the AUC of the mRNA in the human subject is about 10 μg×h/mL to about 100 μg×h/mL. In some embodiments, the AUC of the mRNA in the human subject is about 10 μg×h/mL to about 50 μg×h/mL. In some embodiments, the AUC of the mRNA in the human subject is about 10 μg×h/mL to about 20 μg×h/mL. In some embodiments, the AUC of the mRNA in the human subject is about 20 μg×h/mL to about 100 μg×h/mL. In some embodiments, the AUC of the mRNA in the human subject is about 20 μg×h/mL to about 50 μg×h/mL. In some embodiments, the AUC of the mRNA in the human subject is about 20 μg×h/mL to about 30 μg×h/mL. In some embodiments, the AUC of the mRNA in the human subject is about 30 μg×h/mL to about 100 μg×h/mL. In some embodiments, the AUC of the mRNA in the human subject is about 30 μg×h/mL to about 50 μg×h/mL. In some embodiments, the AUC of the mRNA in the human subject is about 50 μg×h/mL to about 100 μg×h/mL.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein administration of the gene editing system results in a plasma Cmax of the amino lipid in the human subject to be about 1 μg/mL to about 100 μg/mL. In some embodiments, the plasma Cmax of the amino lipid in the human subject is about 1 μg/mL to about 50 μg/mL. In some embodiments, the plasma Cmax of the amino lipid in the human subject is about 1 μg/mL to about 30 μg/mL. In some embodiments, the plasma Cmax of the amino lipid in the human subject is about 1 μg/mL to about 20 μg/mL. In some embodiments, the plasma Cmax of the amino lipid in the human subject is about 1 μg/mL to about 10 μg/mL. In some embodiments, the plasma Cmax of the amino lipid in the human subject is about 10 μg/mL to about 100 μg/mL. In some embodiments, the plasma Cmax of the amino lipid in the human subject is about 10 μg/mL to about 50 μg/mL. In some embodiments, the plasma Cmax of the amino lipid in the human subject is about 10 μg/mL to about 30 μg/mL. In some embodiments, the plasma Cmax of the amino lipid in the human subject is about 20 μg/mL to about 100 μg/mL. In some embodiments, the plasma Cmax of the amino lipid in the human subject is about 20 μg/mL to about 50 μg/mL. In some embodiments, the plasma Cmax of the amino lipid in the human subject is about 50 μg/mL to about 100 μg/mL.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein administration of the gene editing system results in an AUC of the amino lipid in the human subject to be about 100 μg×h/mL to about 10000 μg×h/mL. In some embodiments, the AUC of the amino lipid in the human subject is about 100 μg×h/mL to about 5000 μg×h/mL. In some embodiments, the AUC of the amino lipid in the human subject is about 100 μg×h/mL to about 2000 μg×h/mL. In some embodiments, the AUC of the amino lipid in the human subject is about 100 μg×h/mL to about 1000 μg×h/mL. In some embodiments, the AUC of the amino lipid in the human subject is about 100 μg×h/mL to about 500 μg×h/mL. In some embodiments, the AUC of the amino lipid in the human subject is about 500 μg×h/mL to about 10000 μg×h/mL. In some embodiments, the AUC of the amino lipid in the human subject is about 500 μg×h/mL to about 5000 μg×h/mL. In some embodiments, the AUC of the amino lipid in the human subject is about 500 μg×h/mL to about 2000 μg×h/mL. In some embodiments, the AUC of the amino lipid in the human subject is about 500 μg×h/mL to about 1000 μg×h/mL. In some embodiments, the AUC of the amino lipid in the human subject is about 1000 μg×h/mL to about 10000 μg×h/mL. In some embodiments, the AUC of the amino lipid in the human subject is about 1000 μg×h/mL to about 5000 μg×h/mL. In some embodiments, the AUC of the amino lipid in the human subject is about 5000 μg×h/mL to about 10000 μg×h/mL.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein administration of the gene editing system results in a plasma Cmax of the PEG lipid in the human subject to be about 0.1 μg/mL to about 50 μg/mL. In some embodiments, the plasma Cmax of the PEG lipid in the human subject is about 0.1 μg/mL to about 25 μg/mL. In some embodiments, the plasma Cmax of the PEG lipid in the human subject is about 0.1 μg/mL to about 10 μg/mL. In some embodiments, the plasma Cmax of the PEG lipid in the human subject is about 0.1 μg/mL to about 5 μg/mL. In some embodiments, the plasma Cmax of the PEG lipid in the human subject is about 0.1 μg/mL to about 1 μg/mL. In some embodiments, the plasma Cmax of the PEG lipid in the human subject is about 1 μg/mL to about 50 μg/mL. In some embodiments, the plasma Cmax of the PEG lipid in the human subject is about 1 μg/mL to about 25 μg/mL. In some embodiments, the plasma Cmax of the PEG lipid in the human subject is about 1 μg/mL to about 10 μg/mL. In some embodiments, the plasma Cmax of the PEG lipid in the human subject is about 10 μg/mL to about 50 μg/mL. In some embodiments, the plasma Cmax of the PEG lipid in the human subject is about 10 μg/mL to about 25 μg/mL. In some embodiments, the plasma Cmax of the PEG lipid in the human subject is about 25 μg/mL to about 50 μg/mL.

Described herein is a pharmaceutical composition for a human subject comprising: (a) a mRNA encoding a base editor protein, wherein the base editor protein comprises a nucleic acid binding domain, (b) a guide RNA that serves to guide the nucleic acid binding domain to a protospacer sequence on a target gene, and (c) a LNP comprising an amino lipid, a PEG lipid, a phospholipid and a sterol, wherein administration of the gene editing system results in an AUC of the PEG lipid in the human subject to be about 10 μg×h/mL to about 5000 μg×h/mL. In some embodiments, the AUC of the PEG lipid in the human subject is about 10 μg×h/mL to about 2000 μg×h/mL. In some embodiments, the AUC of the PEG lipid in the human subject is about 10 μg×h/mL to about 1000 μg×h/mL. In some embodiments, the AUC of the PEG lipid in the human subject is about 10 μg×h/mL to about 500 μg×h/mL. In some embodiments, the AUC of the PEG lipid in the human subject is about 10 μg×h/mL to about 100 μg×h/mL. In some embodiments, the AUC of the PEG lipid in the human subject is about 100 μg×h/mL to about 5000 μg×h/mL. In some embodiments, the AUC of the PEG lipid in the human subject is about 100 μg×h/mL to about 2000 μg×h/mL. In some embodiments, the AUC of the PEG lipid in the human subject is about 100 μg×h/mL to about 1000 μg×h/mL. In some embodiments, the AUC of the PEG lipid in the human subject is about 100 μg×h/mL to about 500 μg×h/mL. In some embodiments, the AUC of the PEG lipid in the human subject is about 500 μg×h/mL to about 5000 μg×h/mL. In some embodiments, the AUC of the PEG lipid in the human subject is about 500 μg×h/mL to about 2000 μg×h/mL. In some embodiments, the AUC of the PEG lipid in the human subject is about 500 μg×h/mL to about 1000 μg×h/mL. In some embodiments, the AUC of the PEG lipid in the human subject is about 1000 μg×h/mL to about 5000 μg×h/mL. In some embodiments, the AUC of the PEG lipid in the human subject is about 1000 μg×h/mL to about 2000 μg×h/mL. In some embodiments, the AUC of the PEG lipid in the human subject is about 2000 μg×h/mL to about 5000 μg×h/mL.

In some embodiments, the phospholipid is distearoylphosphatidylcholine (DSPC). In some embodiments, the sterol is cholesterol. In some embodiments, the LNP comprises a N-acetylgalactosamine (GalNAc) lipid receptor targeting conjugate.

Described herein is a method for treating or preventing an atherosclerotic cardiovascular disease in a human subject in need thereof, the method comprising administering to the subject a therapeutically effective amount of pharmaceutical composition of any one of claims 116-219.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

Novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 presents data from an in vitro potency study illustrating the percent editing of PCSK9 gene in primary human hepatocytes (PHH) as comparted to primary non-human primate hepatocytes (e.g., primary cyno hepatocytes (PCH)) using an LNP PCSK9 base editor formulation comprising a payload of mRNA MA004 and guide RNA GA346 at 1:1 mRNA:gRNA weight ratio and titrated at different concentrations against multiple lots of PHH and PCH cells. As illustrated in FIG. 1, the administered PCSK9 base editor formulation appears more potent in human than non-human primate hepatocytes.

FIG. 2 illustrates a study protocol using LNP PCSK9 base editor formulation comprising a payload of mRNA MA004 and guide RNA GA346 at 1:1 mRNA:gRNA weight ratio involving a first group of 4 NHPs intravenously infused with a single dose of 0.75 mg/kg, a second group of 22 NHPs intravenously infused with a single dose of 1.5 mg/kg, and third group (“vehicle control”) of 10 NHPs intravenously infused with a single dose of the same LNP delivery vehicle without the gRNA and mRNA drug substance payload. The dose is measured by the total weight of mRNA and gRNA over the weight of the subject.

FIGS. 3-7 is data obtained from the study described in FIG. 2 setting forth PCSK9 editing percent change from baseline through liver biopsy assessing whole liver DNA editing at day 15 post infusion and PCSK9 protein reduction at day 14 post infusion (FIG. 3), PCSK9 protein reduction over an extended period of time post infusion (FIG. 4), LDL-C reduction over an extended period of time post infusion (FIG. 5), ALT enzyme levels over an extended period of time post infusion (FIG. 6), and fasting glucose levels over an extended time period post infusion (FIG. 7).

FIGS. 8-10 illustrate a mouse study protocol (FIG. 8) and data generated from the study supporting the durability of liver editing using the PCSK9 base editor LNP formulations described herein in regenerated PCSK9 edited liver cells post intravenous infusion (FIGS. 9-10). Each dot in the graphs depicted in FIGS. 8-9 represents a mouse.

FIG. 11 illustrates data from an NHP study that shows no evidence of editing in sperm from sexually mature male NHPs post intravenous infusion of PCSK9 base editor LNP formulation described herein.

FIGS. 12-15 is a study protocol and data generated therefrom assessing potential off-target candidate sites of PCSK9 base editor formulations described herein. FIG. 12 is a schematic illustrating the experimental methods used for evaluating the presence of actual off-target editing by the PCSK9 base editor formulations described herein. FIG. 13 illustrates the high sensitivity of the validation assay employed. FIGS. 14-15 are jitter plots generated from the off-target protocol and the validation assay employed showing that there is no detectable off target editing in primary human liver and spleen cells among the top 244 off target candidate sites identified.

FIG. 16 illustrates data from a bio-distribution study of NHPs intravenously dosed with single 1 mg/kg LNP constituted with an ABE mRNA MA004 and guide RNA GA346. The NHPs were sacrificed on day 15 post infusion and tissue samples taken and analyzed for adenine editing of the PCSK9 gene. PCSK9 editing was observed to be predominately distributed to the liver.

FIG. 17 illustrates the potency of disclosed gRNA variants directed to ANGPTL3 and their on-target editing percentage of ANGPTL3 at position 6 in human primary hepatocytes. It should be understood that the “position 6,” as used in the data referencing position 6, refers to the 6th position counting from the 5′ end of the protospacer sequence (i.e., the opposite end from the PAM). The gRNA variants were transfected into human primary hepatocyte cells together with ABE mRNA MA004 at the concentrations noted therein in a manner described herein.

FIG. 18 illustrates the potency truncated guide RNA GA441 (n-1: GA475, n-2: GA576, n-3: GA477) and their on-target editing percentage of ANGPTL3 at the desired editing position in Human primary hepatocytes combined with the observed corresponding reduction of potential off-target editing at selected off-target candidate sites (OT #1, OT #2, OT #3, OT #4). The gRNA truncated variants were transfected into human primary hepatocyte cells together with mRNA MA004 in a manner described herein and illustrate how such truncated variants of gRNA GA441 impact potential off-target sites more than on-target editing.

FIG. 19 shows exemplary modified ABE variants transfected with gRNA GA441 and their on-target editing percentage of ANGPTL3 at position 6 in primary human hepatocytes (PHH) transfected with gRNA GA441 together with the identified ABE variant at the noted concentrations in a manner described herein. Each sample was normalized to the respective MA004 ABE mRNA sample per concentration. Each set of columns in the illustrated graph correspond to MA004, MA040, MA041, MA045, MA064, MA065, MA066, and MA067, in order, as read from left to right as further represented by the hash marks in the legend.

FIGS. 20A-20E show data generated using various ANGPTL3 gRNAs and ABE mRNAs mRNA MA004 and variants thereof. MA040, MA041 and MA045 to illustrate the relative potency of each combination of gRNA and mRNA vis-à-vis on-target editing of ANGPTL3 in human primary hepatocytes transfected at the noted concentrations. FIG. 20A, FIG. 20B, and FIG. 20C are results from experiment 1 using MA004 encoding ABE8.8, MA040 encoding ABE8.8 D1135E, and MA041 encoding ABE8.8 R691A/D1135E, respectively, with ANGPTL3 targeted gRNA GA441, GA442, GA472, GA473, GA474, GA475, GA476 and GA477 as specified in the gRNA legend and illustrated in the graphs in order from left to right. FIG. 20D and FIG. 20E are results from experiment 2 using MA004 encoding ABE8.8, and MA045 encoding ABE8.8 R691A, respectively.

FIG. 21 is a comparative graph illustrating the percent on target editing of PCSK9 of human primary hepatocytes transfected with the referenced gRNA variants and ABE mRNA MA004 at the noted 4 concentrations. The graph illustrates the relative potency between different gRNAs in human primary hepatocytes. From left to right of the graph, the gRNA variants in the 4 concentrations are GA346, GA376, GA377, GA380, GA381, GA382, GA383, GA384, GA385, GA386, GA387, GA388, GA389, and GA391 respectively.

FIG. 22 is a comparative graph illustrating the percent on target editing of PCSK9 of cyno primary hepatocytes transfected with the referenced gRNA variants and ABE mRNA MA004 at the noted 4 concentrations. The graph illustrates the relative potency in primary hepatocytes between spacer and tracr chemistries variants of gRNA GA346 embodied in the referenced gRNA variants. From left to right of the graph, the gRNA variants in the 4 concentrations are GA346, GA376, GA377, GA380, GA381, GA382, GA383, GA384, GA385, GA386, GA387, GA388, GA389, and GA391 respectively.

FIG. 23 is a comparative graph illustrating the percent on-target editing of PCSK9 of primary hepatocyte transfected with ABE mRNA M004 in combination with gRNAs GA376, GA377, GA380-GA389, GA391 and GA066 (from left to right) between guide concentration of 312.5 ng/TA/mL to 2500 ng/TA/mL. Four different lots of GA066 were used in this study. GA376, GA377, GA380, GA383, GA385 and GA386 showed comparable dose response to GA066. The graph illustrates the relative potency between the gRNAs in human primary hepatocytes.

FIG. 24 is a table illustrating predicted exposures for LNP constituted with an ABE mRNA MA004 and guide RNA GA346 in Humans. Pharmacokinetic (“PK”) modeling using the NHP data disclosed herein was used to predict exposure correlations in human subjects and potential safety margin. Abbreviations: AUC=area under the plasma concentration-time curve; plasma Cₘₐₓ=plasma maximal concentration; NHP=non-human primate. The modeling employed the following assumption:
(a) A graded infusion: the first 15 minutes the infusion rate (in terms of volume) is 1 mL/min; for the remaining infusion duration, the infusion rate is 3 mL/min.
(b) Predicted exposure for a 75 kg person.
(c) For NHP, 2 mg/kg (single dosing plasma Cmax and AUC_{inf} were the mean values.

FIG. 25 is a table showing the percent reduction in PCSK9 human subjects at different doses of an ABE mRNA MA004 and guide RNA 346 formulated in an LNP described herein based on predictive PK-PD modeling and broken out by human subject body weight and equivalent and 3-fold higher potency in humans than NHPs. PCSK9% reduction=100%−PCSK9% remaining relative to baseline. This is just one method of predicting the human dosing and other factors might affect the particular mRNA used and the assay implemented.

FIG. 26 shows relative editing percentage of an off-target candidate site B2 in primary human hepatocytes transfected with the referenced ABE mRNA and the noted ANGPTL3 guide RNAs GA441, GA475 and GA476. The relative editing percentage of each sample was normalized to the GA441/MA004 sample for site B2.

FIG. 27 shows relative editing percentage of an off-target candidate site B6 in primary human hepatocytes transfected with the referenced ABE mRNA and the noted ANGPTL3 guide RNAs GA441, GA475 and GA476. The relative editing percentage of each sample was normalized to the GA441/MA004 sample for site B6.

FIG. 28A illustrates an exemplary scheme of the gene editing system comprising a guide nucleic acid. The guide nucleic acid comprises a spacer sequence with a first exemplary motif gRNAs GA837, GA693 and GA749 disclosed herein are embodiments of this motif.

FIG. 28B illustrates another exemplary scheme of the gene editing system comprising a guide nucleic acid. The guide nucleic acid comprises a spacer sequence with a second exemplary motif gRNAs GA745, GA692 and GA748 disclosed herein are embodiments of this motif.

FIG. 28C illustrates another exemplary scheme of the gene editing system comprising a guide nucleic acid. The guide nucleic acid comprises a spacer sequence with a third exemplary motif gRNAs GA682, GA723 and GA746 disclosed herein are embodiments of this motif.

FIG. 28D illustrates another exemplary scheme of the gene editing system comprising a guide nucleic acid. The guide nucleic acid comprises a spacer sequence with a fourth exemplary motif gRNAs GA691, GA724 and GA747 disclosed herein are embodiments of this motif.

FIG. 29 shows the percent on target editing of the target gene ANGPTL3 results from the initial on-target in vitro screening for the guide nucleic acids comprising the spacer sequences (guide nucleic acids GA675-GA684) under MessengerMax transfection conditions. GA441 was used as a control. Primary human hepatocyte (PHH) cells (lot STL) were used for this study. Each guide was screened at the following concentrations: 5,000 ng/mL, 2,500 ng/mL, 1,250 ng/mL, and 625 ng/mL, the results of which are represented by the columns in decreasing concentration order from left to right in the graph for each guide.

FIG. 30 shows the percent on target editing of the target gene ANGPTL3 results from the initial on-target screening for the guide nucleic acids comprising the spacer sequences (guide nucleic acids GA685-GA694) and control guide RNA GA441 under MessengerMax transfection conditions in PHH cells (lot STL). Each guide was screened at the following concentrations: 5,000 ng/mL, 2,500 ng/mL, 1,250 ng/mL, and 625 ng/mL, the results of which are represented by the columns in decreasing concentration order from left to right in the graph for each guide.

FIG. 31 shows the results of the in vitro off-target (OT1) editing based on ANGPTL3 guide nucleic acids comprising the spacer sequences of GA675-GA684 and control guide RNA GA441 under MesssengerMax transfection conditions in PHH cells (lot STL).

FIG. 32 shows the results of the in vitro off-target (OT1) editing based on ANGPTL3 guide nucleic acids comprising the spacer sequences of GA685-GA694 and control guide RNA GA441 under MesssengerMax transfection conditions in PHH cells (lot STL).

FIG. 33 shows the results from the initial in vitro on-target screening for ANGPTL3 guide nucleic acids comprising the additional spacer sequences of GA695-GA715 and control guide RNA GA441 by MessengerMax transfection conditions in PHH cells (lot STL).

FIG. 34 shows the in vitro on-target and select off-target (OT1 and OT3 sites) editing results from a follow-up experiment with guide nucleic acids (GA675, GA677, GA678, GA680, GA682, GA683, GA685-GA695) comprising selected spacer sequences and ANGPTL3 control guide RNA GA441 by MessengerMax transfection in PHH cells (lot IRZ).

FIG. 35A shows the results of a SureSelect in vitro study of target and off-target (OT1, OT2, OT3 and other OTs) editing, using 50 ng DNA input, of 16 ANGPTL3 guide nucleic acids comprising selected spacer sequences and ANGPTL3 control guide RNA GA441 under MessengerMax transfection conditions in PHH cells (lots IRZ).

FIG. 35B shows the results of a SureSelect in vitro study of on-target and off-target (OT1, OT2, OT3 and other OTs) editing, using 150 ng DNA input, of 10 ANGPTL3 guide nucleic acids comprising selected spacer sequences and ANGPTL3 control guide RNA GA441 under MessengerMax transfection conditions in PHH cells (lot IRZ).

FIGS. 36A-36B shows the results from LNP transfection experiments using various ANGPTL3 guides. FIG. 36A shows the on-target in vitro editing results from the use of LNPs for transfection in PHH cells (lot IRZ) at two different doses of LNP. FIG. 36B shows the off-targeting editing obtained from the same LNPs transfection experiment for only one dose, except for the control. The LNPs used in these studies are described in Table. 5.

FIG. 37 shows the results of a SureSelect in vitro study of ANGPTL3 on-target and off-target (OT1, OT2, OT3 and other OTs) editing resulting from the LNP transfection experiments in PHH cells (lot IRZ). The LNPs used in these studies are described in Table. 5.

FIG. 38A shows a subset of the SureSelect in vitro on-target and off-target (OT1, OT2, OT3 and other OTs) editing results of the LNP transfection experiments in PHH cells (lot IRZ). The LNPs used in these studies are described in Table. 5.

FIG. 38B shows the Targeted Amplicon in vitro on-target and OT1 editing result of the same subset data, of the LNP transfection experiments.

FIG. 39A shows a subset of the SureSelect in vitro on-target and off-target (OT1, OT2, OT3 and other OTs) editing results of the MessengarMax transfection experiments in PHH cells (lot IRZ).

FIG. 39B shows the Targeted Amplicon in vitro on-target and off-targets OT1 and OT3 editing result of the same subset data of the MessengarMax transfection experiments.

FIG. 40 shows the results of an in vitro on-target dose-response study of ANGPTL3 editing produced by different LNP formulations comprising spacer-modified ANGPTL3 guide nucleic acids in PHH cells (lot IRZ) assessed using Targeted Amplicon sequencing. Three digits after decimal point following formulation, gRNA and mRNA IDs indicate lot or batch number of the respective formulation, gRNA and mRNA. The LNPs used in these studies are described in Table 5.

FIGS. 41A-41B is a comparative illustration of the off-target editing percentages data produced by different LNP formulations comprising ANGPTL3 guide nucleic acids with various modified-spacer sequences as specified and described herein. FIG. 41A shows the in vitro off-target editing results using OT1 primer. FIG. 41B shows the off-target editing results using OT3 primer. Three digits after decimal point following formulation, gRNA and mRNA IDs indicate lot or batch number of the respective formulation, gRNA and mRNA. The LNPs used in these studies are described in Table 5.

FIG. 42 shows the results of an in vitro on-target dose-response study of ANGPTL3 editing using an LNP containing GA837 and MA079 in PHH cells from four different donors (lots NFX, GNA, IRZ, and LFQ) assessed using Targeted Amplicon sequencing. The LNP (VF1542) is described in Table 5.

FIG. 43A shows results of an in vitro on-target ANGPTL3 editing results (as net editing of treated minus untreated samples) of LNP, containing GA837 and MA079, transfected at a single dose in PHH cells (lot IRZ) for two biological replicates. The average net editing % shown above each bar is derived from two technical replicates. The LNP (VF1542) is described in Table 5.

FIG. 43B shows the OGM (optical genome mapping) analysis of genomic DNA from one replicate (Replicate 1) of the same subset of ANGPTL3 editing data of FIG. 43A. Circos plots show all SV (structural variations) categories (insertions, duplications, deletions, inversions, and translocations) for the individual Untreated Control (left) and ABE-treated (i.e., LNP (VF1542)-treated) (middle) samples. The Bionano Dual analysis pipeline was used to identify SVs unique to the treatment condition. As indicated by the clear ABE-Untreated Control Circos plot (right), notably no unique SVs were identified. The gRNA, mRNA and LNP formulations are the same as FIG. 43A.

FIG. 44A shows the hepatic ANGPTL3 on-target editing by selected (cyno equivalent) spacer-modified guide nucleic acids GA347, GA663, GA666, GA668, GA665, GA720 comprising the same spacer sequences and mRNA MA004 in LNP treated NHPs. FIG. 44B shows the corresponding ANGPTL3 protein reduction 15 days after a single dose IV administration of the LNPs. The LNPs used in this study are described in Table 6.

FIG. 45 shows the in vitro on-target editing percentage by selected (cyno equivalent) spacer-modified guide nucleic acids GA347, GA663, GA666, GA668, GA665, GA720 in LNPs in PCH. Corresponding human gRNA IDs with equivalent spacer sequences are listed at the bottom. The LNPs used in this study are described in Table 6.

FIG. 46 shows hepatic ANGPTL3 on-target editing in NHP after IV infusion a single dose of LNP constituted with selected spacer-modified guide nucleic acids GA347, GA668, GA748 and GA749 comprising the same spacer sequences and mRNA MA079. The editing was assayed 15 days after injection to Cynomolgus monkeys, achieved >54% liver ANGPTL3 editing. The LNPs used in this study are described in Table 6.

FIG. 47 shows the spacer-modified guide nucleic acids comprising same spacer sequences achieved significant reduction in plasma ANGPTL3 protein 15 days post dosing in NHP. All spacer-modified guide nucleic acid produced >90% reduction in plasma ANGPTL3 protein at all dose level evaluated. The LNPs used in this study are described in Table 6.

### DETAILED DESCRIPTION

Certain specific details of this description are set forth in order to provide a more thorough understanding of various aspects and embodiments. However, one skilled in the art will understand that the present disclosure may be practiced without certain of the details herein disclosed. In other instances, well-known structures have not been shown or described in detail to avoid unnecessarily obscuring descriptions of the embodiments.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below. Further, headings provided herein are for convenience only and do not interpret the scope or meaning of the claimed disclosure. It is contemplated that any embodiment discussed in this specification can be implemented with respect to any method or composition of the present disclosure, and vice versa. Furthermore, compositions of the present disclosure can be used to achieve methods of the present disclosure.

### Definitions

To facilitate an understanding of the present disclosure, a number of terms and phrases are defined below.

As used in this specification and the appended claims, the singular forms “a,” “an,” and “the” include plural referents unless the content clearly dictates otherwise.

It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “some embodiments,” “an embodiment,” “one embodiment,” “embodiments” or “other embodiments” means that a particular feature, structure, or characteristic described in connection with the embodiments is included in at least some embodiments, but not necessarily all embodiments, of the present disclosures.

The term “nucleic acid” as used herein refers to a polymer containing at least two nucleotides (i.e., deoxyribonucleotides or ribonucleotides) in either single- or double-stranded form and includes DNA and RNA, hybrids of DNA and RNA, and combinations thereof. The term “nucleic acid” as used herein also refers to a polymer containing at least two chemically modified nucleotides (i.e., deoxyribonucleotides or ribonucleotides) in either single- or double-stranded form and includes DNA and RNA, hybrids of DNA and RNA, and combinations thereof.

The term “nucleotide” refers to a molecule that contains a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

A nucleic acid includes any oligonucleotide or polynucleotide, with fragments containing up to 60 nucleotides generally termed oligonucleotides, and longer fragments termed polynucleotides. A deoxyribo-oligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and inter-sugar (backbone) linkages. Additionally, nucleic acids include nucleic acids containing known nucleotide analogs or modified backbone residues or linkages, which are synthetic, nonstandard, and/or non-naturally occurring, and which have similar binding properties as the reference nucleic acid. The nucleic acid may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delays nuclease degradation of oligonucleotides. A phosphorodiamidate linkage (N3′∝P5′) allows preventing nuclease recognition and degradation. Backbone modifications can also include having peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

Nucleic acid molecules described herein may contain a sugar moiety that comprises ribose or deoxyribose, as present in naturally occurring nucleotides, or a modified sugar moiety or sugar analog. The examples of modified sugar moieties include, but are not limited to, 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino). Examples of such analogs and/or modified residues include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, methyl phosphonates, chiral-methyl phosphonates, 2′-O-methyl ribonucleotides, peptide-nucleic acids (PNAs), and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Such modified or substituted oligonucleotides are often preferred over native forms because of properties such as, for example, enhanced cellular uptake, reduced immunogenicity, and increased stability in the presence of nucleases. Thus, the terms “polynucleotide” and “oligonucleotide” can also include polymers or oligomers comprising non-naturally occurring monomers, or portions thereof, which function similarly.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “nucleoside” refers to a compound that consists of a base combined with deoxyribose or ribose. Nucleosides include but not limited to, ribonucleoside and deoxyribonucleoside. Nucleosides are phosphorylated to give nucleotides. Ribonucleosides include adenosine (A), guanosine (G), 5-methyluridine (m⁵U), uridine (U), and cytidine (C). Deoxyribonucleosides include deoxyadenosine (dA), deoxyguanosine (dG), deoxythymidine (dT), deoxyuridine (dU), deoxycytidine (dC). The deoxyribose or ribose (i.e., sugar moieties) can be modified. The examples of modified sugar moieties include, but are not limited to, 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino). Examples of such analogs and/or modified residues include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, methyl phosphonates, chiral-methyl phosphonates, 2′-O-methyl ribonucleotides, peptide-nucleic acids (PNAs), and the like.

The term “deoxyribonucleotide” and “2′-deoxyribonucleotide” are used interchangeably and refer to both unmodified deoxyribonucleotide and chemically modified deoxyribonucleotide unless otherwise specified.

The term “motif,” as used herein, refers to a pattern or an arrangement of specific nucleotides, for example, a motif can refers to (i) one or more 2′-deoxyribonucleotides within a spacer, (ii) a combination of one or more 2′-deoxyribonucleotides and one or more ribonucleotides within the spacer, (iii) a combination of one or more 2′-deoxyribonucleotides, one or more ribonucleotides, and one or more 2′-OMe ribonucleotides within the spacer, wherein the 2′-OMe ribonucleotide can be at the 5′-end or at the 3′-end of a 2′-deoxyribonucleotide, (iv) a combination of one or more 2′-deoxyribonucleotides and one or more 2′-OMe ribonucleotides within the spacer, wherein the 2′-OMe ribonucleotide can be placed/located at the 5′-end or at the 3′-end of the 2′deoxynucleotides, or (v) any of the motifs disclosed earlier (i-iv) further comprising a phosphonothioate backbone.

The term “vector,” as used herein, refers to a nucleic acid molecule capable of transporting another nucleic acid to which it has been linked. In some examples, a vector is an expression vector that is capable of directing the expression of nucleic acids to which they are operatively linked. The term “operably linked,” as used herein, means that the nucleotide sequence of interest is linked to regulatory sequence(s) in a manner that allows for expression of the nucleotide sequence. The term “regulatory sequence,” as used herein, includes, but is not limited to promoters, enhancers and other expression control elements. Such regulatory sequences are well known in the art and are described, for example, in Goeddel; Gene Expression Technology: Methods in Enzymology 185, Academic Press, San Diego, CA (1990). Examples of expression vectors include, but are not limited to, plasmid vectors, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors.

The term “off-targeting,” “off-site targeting,” or “off-target effect” as used herein refers to when the spacer of the guide nucleic acid binds to a sequence of the genome other than the target sequence to which the spacer was specifically designed to bind. The resulting unintentional binding would lead to unintentional editing of genes other than the target gene.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

The term “sequence identity,” as used herein, refers to the amount of nucleotide or amino acid which match exactly between two different sequences. When comparing RNA and DNA sequences Uracil and Thymine bases are considered to be the same base. Gaps are not counted and the measurement is typically in relation to the shorter of the two sequences.
For example, for nucleotide sequences:
A: AAGGCTT
B: AAGGC
C: AAGGCAT
Here identity (A,B)=100% (5 identical nucleotides/min(length(A),length(B))).

Identity(B,C)=100%, but identity(A,C)=85% ((6 identical nucleotides/7). So 100% identity does not mean two sequences are the same.

For amino acid sequences:
A: Ala Ala Gly Gly Gln His His
B: Ala Ala Gly Gly Gln
C: Ala Ala Gly Gly Gln Ala His
Here identity (A,B)=100% (5 identical amino acids/min(length(A), length(B))).
Identity (B,C)=100%, but identity(A,C)=85% ((6 identical amino acids/7).

The term “sequence similarity,” as used herein, can be described as an optimal matching problem that finds the minimal number of edit operations (inserts, deletes, and substitutions) in order to transform the one sequence into an exact copy of the other sequence being aligned (edit distance). Using this, the percentage sequence similarity of the examples above are sim(A,B)=60%, sim(B,C)=60%, sim(A,C)=86% (semi-global, sim=1−(edit distance/unaligned length of the shorter sequence)).

A “subject” in need thereof, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hypercholesterolemia. In some embodiments, the subject has atherosclerotic vascular disease. In some embodiments, the subject has hypertriglyceridemia. In some embodiments, the subject has diabetes. The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “condition,” as used herein, includes diseases, disorders, and susceptibilities. In some embodiments, the condition is an atherosclerotic vascular disease. In some embodiments, the condition is a hypertriglyceridemia. In some embodiments, the condition is a diabetes.

The term “atherosclerosis” or “atherosclerotic vascular disease,” as used herein, refers to a disease in which the inside of an artery narrows due to the buildup of plaque. In some instances, it may result in coronary artery disease, stroke, peripheral artery disease, or kidney problems.

The term “hypertriglyceridemia,” as used herein, refers to high (hyper-) blood levels (-emia) of triglycerides, the most abundant fatty molecule in most organisms. Elevated levels of triglycerides can be associated with atherosclerosis, even in the absence of hypercholesterolemia (high cholesterol levels), and can predispose to cardiovascular disease. Very high triglyceride levels can increase the risk of acute pancreatitis. Hypertriglyceridemia can be associated with overeating, obesity, diabetes mellitus and insulin resistance, excess alcohol consumption, kidney failure, nephrotic syndrome, genetic predisposition (e.g., familial combined hyperlipidemia, i.e., Type II hyperlipidemia), lipoprotein lipase deficiency, lysosomal acid lipase deficiency, cholesteryl ester storage disease, certain medications (e.g., isotretinoin, hydrochlorothiazide diuretics, beta blockers, protease inhibitors), hypothyroidism (underactive thyroid), systemic lupus erythematosus and associated autoimmune responses, glycogen storage disease type 1, propofol, or HIV medications.

The term “diabetes,” as used herein, refers to a group of metabolic disorders characterized by a high blood sugar level over a prolonged period of time. Diabetes can be type 1 diabetes that results from the pancreas's failure to produce enough insulin due to loss of beta cells. Diabetes can be type 2 diabetes characterized by insulin resistance, a condition in which cells fail to respond to insulin properly. Diabetes can be gestational diabetes that occurs when pregnant women without a previous history of diabetes develop high blood sugar levels.

The term “low-density lipoprotein (LDL),” as used herein, refers to a microscopic blob made up of an outer rim of lipoprotein and a cholesterol center. LDL can have a highly hydrophobic core composed of a polyunsaturated fatty acid known as linoleate and hundreds to thousands esterified and unesterified cholesterol molecules. The core of LDL can also carry triglycerides and other fats and can be surrounded by a shell of phospholipids and unesterified cholesterol.

The term “high-density lipoprotein (HDL),” as used herein, refers to the smallest lipoprotein particles. Plasma enzyme lecithin-cholesterol acyltransferase (LCAT) can convert the free cholesterol into cholesteryl, which is then sequestered into the core of the lipoprotein particle, eventually causing the newly synthesized HDL to assume a spherical shape. HDL particles can increase in size as they circulate through the bloodstream and incorporate more cholesterol and phospholipid molecules from cells and other lipoproteins.

The term “cholesterol,” as used herein, refers to a lipid with a unique structure composed of four linked hydrocarbon rings forming the bulky steroid structure. The term “triglyceride,” as used herein, refers to a tri-ester composed of a glycerol bound to three fatty acid molecules. In some embodiments, the fatty acids are saturated or unsaturated fatty acids.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., delaying, decreasing, suppressing, attenuating, diminishing, arresting, or stabilizing the development or progression of a disease or the condition, relieving the disease or the condition, causing regression of the disease or the condition, relieving a condition caused by the disease or the condition, reducing disease severity, or stopping the symptoms of the disease or the condition either prophylactically and/or therapeutically. “Treating” also includes lessening the frequency of occurrence or recurrence, or the severity, of any symptoms or other ill effects related to a disease or condition and/or the side effects associated with the disease or condition. “Treating” does not necessarily require curative results. It is appreciated that, although not precluded, treating a disorder or condition also does not require that the disorder, condition, or symptoms associated therewith be completely eliminated. The term “treating” encompasses the concept of “managing” which refers to reducing the severity of a particular disease or disorder in a patient or delaying its recurrence, e.g., lengthening the period of remission in a patient who had suffered from the disease. “Treating” may refer to the application or administration or a composition to a subject after the onset, or suspected onset, of a disease or condition.

The term “treating” further encompasses the concept of “prevent,” “preventing,” and “prevention.” The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

By “treating or preventing a condition,” for example, as compared with an equivalent untreated control, alleviating a symptom of a disorder may involve reduction or degree of prevention at least 3%, 5%, 10%, 20%, 40%, 50%, 60%, 80%, 90%, 95%, 98%, 99%, 99.5%, 99.9%, or 100% as measured by any standard technique. In some embodiments, alleviating a symptom of a disorder may involve reduction or degree of prevention by at least 2, 3, 4, 5, 10, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 fold as compared with an equivalent untreated control.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence.

“Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The term “parenteral” as used herein includes subcutaneous, intracutaneous, intravenous, intramuscular, intraperitoneal, intradermal, intraarterial, intrasynovial, intrasternal, intrathecal, intravascular, intralesional, and intracranial injection or infusion techniques. In addition, it can be administered to the subject via injectable depot routes of administration such as using 1-, 3-, or 6-month depot injectable or biodegradable materials and methods.

By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

“A therapeutically effective amount” as used herein refers to the amount of each composition of the present disclosure required to confer therapeutic effect on the subject, either alone or in combination with one or more other therapeutic agents. Hence, as used herein, the term “therapeutically effective amount” means an amount of an agent to be delivered (e.g., nucleic acid, composition, therapeutic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition. In terms of treatment, a “therapeutically effective amount” is an amount that is sufficient to palliate, ameliorate, stabilize, reverse or slow the progression of a disease or a condition, e.g., an atherosclerotic vascular disease, hypertriglyceridemia, or diabetes. A “therapeutically effective amount” varies, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Additionally, other medication the patient may be receiving will affect the determination of the therapeutically effective amount of the therapeutic agent to administer. Empirical considerations, such as the half-life, generally will contribute to the determination of the dosage. A “therapeutically effective amount” may be of any of the compositions of the disclosure used alone or in conjunction with one or more agents used to treat a condition. A therapeutically effective amount can be administered in one or more administrations.

An effective initial method to determine a “therapeutically effective amount” may be by carrying out cell culture assays (for example, using neuronal cells) or using animal models (for example, mice, rats, rabbits, dogs or pigs). A dose may be formulated in animal models to achieve a concentration range that includes the IC50 (i.e., the concentration of the composition which achieves a half-maximal inhibition of symptoms) as determined in cell culture. Such information can be used to more accurately determine useful doses in humans. In addition to determining the appropriate concentration range for an disclosure composition to be therapeutically effective, animal models may also yield other relevant information such as preferable routes of administration that will give maximum effectiveness. Adjusting the dose to achieve maximal efficacy in humans based on the methods described above and other methods is well within the capabilities of the ordinarily skilled artisan.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “protospacer,” or “target sequence” and their grammatical equivalents as used herein can refer to a DNA sequence of a target gene. In the native state, a protospacer is adjacent to a PAM (protospacer adjacent motif). The site of cleavage by an RNA-guided nuclease is within a protospacer sequence.

The term “spacer” or “spacer sequence” refers to a nucleic acid sequence that is complementary and binds to the complementary strand of the target gene. A spacer can be within a guide nucleic acid (e.g., gRNA, gDRNA).

The term “base editing,” “gene editing,” “genome editing,” or “gene modification” and its grammatical equivalents as used herein can refer to genetic engineering in which one or more nucleotides are inserted, replaced, or removed from a genome. Gene editing can be performed using a nuclease (e.g., a natural-existing nuclease or an artificially engineered nuclease). Gene modification can include introducing a double stranded break, a non-sense mutation, a frameshift mutation, a splice site alteration, or an inversion in a polynucleotide sequence, e.g., a target polynucleotide sequence.

The term “base editor” as used herein can refer to an agent that binds a polynucleotide and has nucleobase modifying activity. The base editor can comprise a nucleobase modifying polypeptide (e.g., a deaminase) and a nucleic acid programmable nucleotide binding domain in conjunction with a guide polynucleotide (e.g., guide RNA), or nucleic acids encoding the programmable nucleotide binding domain and the deaminase. The agent can be a biomolecular complex comprising a protein domain having base editing activity, i.e., a domain capable of modifying a base (e.g., A, T, C, G, or U) within a nucleic acid molecule (e.g., DNA), or a nucleic acid encoding the same. The polynucleotide programmable DNA binding domain can be fused or linked to a deaminase domain, resulting in a base editor fusion protein. The base editor can comprise a nucleic acid encoding the base editor fusion protein, e.g., a mRNA encoding the base editor fusion protein. The base editor fusion protein can comprise one or more linkers, for example, peptide linkers. The agent can be a fusion protein comprising a domain having base editing activity. The protein domain having base editing activity can be linked to the guide RNA (e.g., via an RNA binding motif on the guide RNA and an RNA binding domain fused to the deaminase). In some instances, the domain having base editing activity is capable of deaminating a base within a nucleic acid molecule. In some instances, the base editor is capable of deaminating one or more bases within a DNA molecule. In some instances, the base editor is capable of deaminating an adenosine (A) within DNA. The base editor can be an adenosine base editor (ABE). In some instances, the base editor is capable of deaminating a cytosine (C) within DNA. The base editor can be a cytosine base editor (CBE).

The term “base editor system” refers to a gene editing system for editing a single nucleobase of a target nucleotide sequence. In various embodiments, the base editor system comprises (1) a polynucleotide programmable nucleotide binding domain (e.g., Cas9); (2) a deaminase domain (e.g., an adenosine deaminase or a cytidine deaminase) for deaminating said nucleobase; and (3) one or more guide polynucleotide (e.g., guide RNA). In some embodiments, the base editor system comprises a base editor fusion protein comprising (1) and (2). In some embodiments, the polynucleotide programmable nucleotide binding domain is a polynucleotide programmable DNA binding domain. In some embodiments, the base editor is an adenine or adenosine base editor (ABE). In some embodiments, the base editor is a cytosine base editor (CBE).

The term “prime editing” as used herein refers to a form of precise genome editing that enables direct, irreversible targeted small insertions, deletions, and base swapping without requiring double-stranded DNA breaks (DSBs), or donor DNA templates. DNA prime editors comprise a catalytically disabled Cas9 nuclease fused to a reverse transcriptase. Prime editing involves a prime editing guide RNA (pegRNA) that is substantially larger than a standard sgRNA used for CRISPR Cas9 editing. The pegRNA comprises a primer binding sequence (PBS) and a template containing the desired RNA sequence added at the 3′ end.

The term “CRISPR RNA” (crRNA) herein refers to an RNA sequence that can form a complex with one or more Cas proteins (e.g., Cas9) and provides DNA binding specificity to the complex. A crRNA provides DNA binding specificity since it contains a “spacer sequence” that is complementary to a strand of a DNA target sequence. A crRNA further comprises a “repeat sequence” (“tracr RNA mate sequence”) encoded by a repeat region of the CRISPR locus from which the crRNA was derived. A repeat sequence of a crRNA can anneal to sequence at the 5′-end of a tracrRNA. crRNA in native CRISPR systems is derived from a “pre-crRNA” transcribed from a CRISPR locus. A pre-crRNA comprises spacer regions and repeat regions; spacer regions contain unique sequence complementary to a DNA target site sequence. Pre-crRNA in native systems is processed to multiple different crRNAs, each with a guide sequence along with a portion of repeat sequence. CRISPR systems utilize crRNA, for example, for DNA targeting specificity.

The term “trans-activating CRISPR RNA” (tracrRNA) herein refers to a non-coding RNA used in type II CRISPR systems, and contains, in the 5′-to-3′ direction, (i) a sequence that anneals with the repeat region of CRISPR type II crRNA and (ii) a stem loop-containing portion (Deltcheva et al., Nature 471:602-607). A modified tracrRNA refers to a tracrRNA with modified ribonucleotide (e.g., 2-OMe modified RNA).

As used herein, the term “guide nucleic acid”, relates to a polynucleotide sequence that can form a complex with a Cas endonuclease and enables the Cas endonuclease to recognize and optionally cleave a DNA target site. The guide nucleic acid can be a single molecule or a double molecule. The guide nucleic acid sequence can be DNA only (gDNA). The DNA can be modified or unmodified. The guide nucleic acid sequence can be RNA only (gRNA). The RNA can be modified or unmodified. The guide nucleic acid sequence can be a combination of DNA and RNA. Optionally, the guide nucleic acid can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide nucleic acid that solely comprises ribonucleic acids is referred to as a “guide RN.” A guide nucleic acid that comprises both RNA and DNA is referred to as a “guide RDNA.”

“Partial hepatectomy,” as used herein, refers to an operation to remove part of the liver. In some embodiments, partial hepatectomy is used to model liver regeneration in vivo.

As used herein, a spacer sequence that corresponds to a protospacer is capable of making a modification to a base within the complimentary strand of the target protospacer nucleic acid sequence. a spacer sequence that corresponds to a protospacer sequence may be identical or substantially identical to the protospacer sequence.

As used herein, “corresponding” refers to a region where a different sequence or component can react to or bind to. For example, a corresponding region of a Cas9 nickase to a scaffold component of a guide RNA refers to a region of the Cas9 nickase that can react and bind to the scaffold component of a guide RNA. For another example, a spacer sequence corresponds to a protospacer sequence refers to the fact that the spacer sequence binds to the protospacer sequence. The binding might have one or more mis-matches.

### Gene Editing Systems

The clustered regularly interspaced short palindromic repeat (CRISPR) system has been adopted for gene editing and has revolutionized the biotechnology and life science space. Despite being a powerful gene editing tool, CRISPR has its own drawbacks. One of the major issues is the off-target mutations caused by the system. (Yin et al., Nature Chemical Biology 14, 311-316 (2018)). It has been found that partially replacing the RNA nucleotides (ribonucleosides) from the guide RNA with DNA nucleotides (deoxyribonucleosides) can lead to decreased off-target activity compared to the unmodified guide RNA. Such DNA-RNA chimera guides are also known as CRISPR hybrid DNA-RNA (chRDNA). However, while the chRDNA can reduce the off-target effect, it often does so by sacrificing the on-target editing efficiency. Thus, there remains an urgent need in finding optimized guide nucleic acids for CRISPR systems that would maintain the powerful on-target editing efficiency with low off-targeting effect, particularly in vivo.

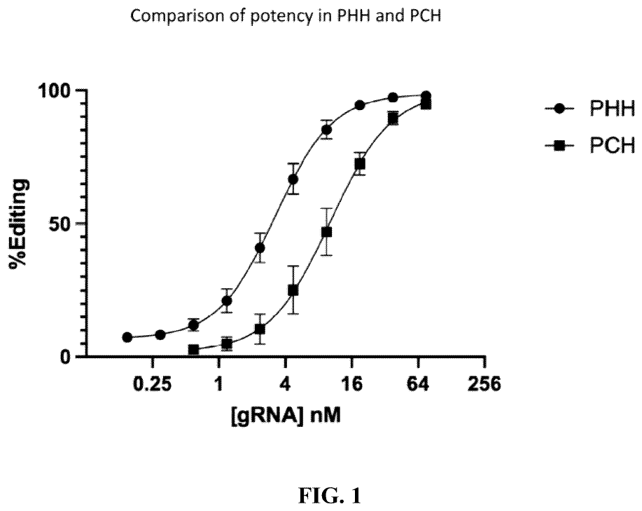

The present disclosure provides a novel gene editing system that offers many of the benefits of using the CRISPR system for gene editing—namely, highly efficient programmable gene editing—while overcoming the limitation of off-target editing. The novel gene editing system described herein achieves efficient gene editing results with reduced off-target effect by using a novel guide nucleic acid to direct a gene editor protein to affect an alteration to a target gene. As disclosed herein, the novel guide nucleic acid is engineered to comprise a mixture of deoxyribonucleotide and ribonucleotide in the spacer sequence comprising at least one deoxyribonucleotide and at least one ribonucleotide in the spacer sequence while retaining its ability to hybridize to the target gene. The number, the position, and the specific modification of the deoxyribonucleotide and/or ribonucleotide in the guide nucleic acid are optimized such that the gene editing system comprising the guide nucleic acid is allowed to perform gene editing with high on-target efficiency but low off-target effect. In some embodiments, the 2′ hydroxyl group on the ribose of the ribonucleotide can be modified to covalently linked to a methyl group. FIGS. 28A-28D provide exemplary gene editing systems with guide nucleic acids comprising exemplary spacer sequences as disclosed herein. As illustrated in FIG. 28A, the exemplary gene editing system (100) comprises a gene editor protein (150) which contains a nucleic acid binding domain that can bind to the target gene (110) and a guide nucleic acid (120). The representative guide nucleic acid (120) comprises a spacer sequence (130) composed of both ribonucleotides (RNA) and deoxyribonucleotides (DNA). The spacer sequence (130) is complementary to a protospacer (160) from the target gene (110). Replacing the ribonucleotides with deoxyribonucleotides at some specific positions of the spacer sequence (130) with 2′-OMe and phosphorothioate backbone chemical modifications results in unique DNA-RNA hybrid sequence motifs at the 5′-end to the spacer sequence (130), which is able to direct the gene editor protein (150) to the target sequence (160) to elicit site-specific on-target gene alteration with reduced off-target effect. Here, the exemplary motif in the spacer sequence (130) is (2′-OMe RNA)(2′-OMe RNA)(DNA)(DNA)(RNA)(DNA)(DNA)(DNA). Note that the specific phosphorothioate backbone modifications are not shown. The guide nucleic acid (120) further comprises a tracrRNA (140) or equivalent thereof at the 3′ end of the spacer sequence (130). The tracrRNA (140) or equivalent thereof recruits a gene editor protein (150) to the target sequence (160) to affect an alteration on the target gene (110). The tracrRNA (140) may be modified. For example, the tracrRNA (140) may comprises a 2′-OMe RNA (not shown). The gene editor protein (150) may comprise a deaminase that can be used for base editing. FIGS. 1B-1D illustrate other exemplary gene editing systems with guide nucleic acids comprising other exemplary spacer sequences. For example, FIG. 28B illustrates a spacer sequence with motif of (2′-OMe RNA)(2′-OMe RNA)(DNA)(DNA)(RNA)(DNA)(DNA). FIG. 28C illustrates another spacer sequence with another motif of (2′-OMe RNA)(2′-OMe RNA)(DNA)(DNA)(DNA)(DNA)(DNA). FIG. 28D illustrates another spacer sequence with another motif of (2′-OMe RNA)(2′-OMe RNA)(2′-OMe RNA)(DNA)(DNA)(DNA)(DNA)(RNA)(DNA)(DNA)(RNA)(RNA)(DNA)(DNA). Note that the specific phosphorothioate backbone modifications are not shown. Note the drawing is for schematic purpose only.

The present disclosure further provides a novel gene editing system for base editing with high on-target efficiency and low off-target editing. The novel gene editing system for base editing described herein achieves the benefits by using the novel guide nucleic acid described herein to direct the gene editor protein comprising a deaminase to modify a nucleobase in the target gene.

The present disclosure further provides a novel gene editing system for editing ANGPTL3 gene with high on-target efficiency and low off-target editing. The novel gene editing system for editing ANGPTL3 gene described herein achieves the benefits by using the novel guide nucleic acid to direct the gene editor protein to affect an alteration to the ANGPTL3 gene.

### CRISPR Cas Systems

There are several different CRISPR/Cas systems and the nomenclature and classification of these have changed as the systems are further characterized. The CRISPR/Cas system is superior to other methods of genome editing involving endonucleases, meganucleases, zinc finger nucleases, and transcription activator-like effector nucleases (TALENs), which may require de novo protein engineering for every new target locus.

Based upon the genes encoding the effector module (i.e., the proteins involved in the interference stage), the CRISPR/Cas systems can be placed in either Class 1 or Class 2. Class 1 systems have a multi-subunit crRNA-effector complex, whereas Class 2 systems have a single protein, such as Cas 9, Cpf1, C2c1, C2c2, C2c3, or a crRNA-effector complex. Class 1 systems comprise Type I, Type III and Type IV systems. Class 2 systems comprise Type II and Type V systems (Makarova et al., Nature Review Microbiology 13: 1-15 (2015)).

Type I systems all have a Cas3 protein with helicase activity and cleavage activity. Type I systems are divided into seven sub-types (I-A to I-F and I-U). Type III systems possess a cas10 gene, which encodes a multidomain protein containing a Palm domain (a variant of the RNA recognition motif (RRM)) that is homologous to the core domain of numerous nucleic acid polymerases and cyclases and that is the largest subunit of type III crRNA-effector complexes. All type III loci also encode the small subunit protein, one Cas5 protein and typically several Cas7 proteins. Type III can be divided into four sub-types, III-A to III-D. Sub-type 111-A has a csm2 gene encoding a small subunit and also has cas1, cas2 and cas6 genes. Sub-type III-B has a cmr5 gene encoding a small subunit and also typically lacks cas1, cas2 and cas6 genes. Sub-type 111-C has a Cas10 protein with an inactive cyclase-like domain and lacks a cas1 and cas2 gene. Sub-type III-D has a Cas10 protein that lacks the HD domain, it lacks a cas1 and cas2 gene and has a cas5-like gene known as csx10. Type IV systems encode a minimal multisubunit crRNA-effector complex comprising a partially degraded large subunit, Csf1, Cas5, Cas7, and in some cases, a putative small subunit. Type IV systems lack cas1 and cas2 genes. Type IV systems do not have sub-types, but there are two distinct variants. One variant has a DinG family helicase, while the other variant lacks a DinG family helicase, but has a gene encoding a small α-helical protein.

Type II systems have cas1, cas2 and cas9 genes. cas9 encodes a multidomain protein that combines the functions of the crRNA-effector complex with target DNA cleavage. Type II systems also encode a tracrRNA. Type II systems are divided into three sub-types, subtypes II-A, II-B and II-C. Sub-type II-A contains an additional gene, csn2. Sub-type II-B lacks csn2, but has cas4. Sub-type II-C is the most common Type II system found in bacteria and has only three proteins, Cas1, Cas2 and Cas9. Type V systems have a cpf1 gene and cas1 and cas2 genes. The cpf1 gene encodes a protein, Cpf1, that has a RuvC-like nuclease domain that is homologous to the respective domain of Cas9, but lacks the HNH nuclease domain that is present in Cas9 proteins.

In Class 1 systems, the expression and interference stages involve multisubunit CRISPR RNA (crRNA)-effector complexes. In these systems, pre-crRNA is bound to the multisubunit crRNA-effector complex and processed into a mature crRNA. In Type I and III systems this involves an RNA endonuclease, e.g., Cas6.

In Class 1 systems the crRNA is associated with the crRNA-effector complex and achieves interference by combining nuclease activity with RNA-binding domains and base pair formation between the crRNA and a target nucleic acid. In Type I systems, the crRNA and target binding of the crRNA-effector complex involves Cas7, Cas5, and Cas8 fused to a small subunit protein. The target nucleic acid cleavage of Type I systems involves the HD nuclease domain, which is either fused to the superfamily 2 helicase Cas3′ or is encoded by a separate gene, cas3″. In Type III systems, the crRNA and target binding of the crRNA-effector complex involves Cas7, Cas5, Cas10 and a small subunit protein. The target nucleic acid cleavage of Type III systems involves the combined action of the Cas7 and Cas10 proteins, with a distinct HD nuclease domain fused to Cas10, which is thought to cleave single-stranded DNA during interferences.

In Class 2 systems, the expression and interference stages involve a single large protein, e.g., Cas9, Cpf1, C2C1, C2C2, or C2C3. In most Class 2 Type II systems, pre-crRNA is bound to Cas9 and processed into a mature crRNA in a step that involves RNase III and a tracrRNA.

In Class 2 systems the crRNA is associated with a single protein and achieves interference by combining nuclease activity with RNA-binding domains and base pair formation between the crRNA and a target nucleic acid. In Type II systems, the crRNA and target binding involves Cas9 as does the target nucleic acid cleavage. In Type II systems, the RuvC-like nuclease (RNase H fold) domain and the HNH (McrA-like) nuclease domain of Cas9 each cleave one of the strands of the target nucleic acid. The Cas9 cleavage activity of Type II systems also requires hybridization of crRNA to tracrRNA to form a duplex that facilitates the crRNA and target binding by the Cas9. In Type V systems, the crRNA and target binding involves Cpf1 as does the target nucleic acid cleavage. In Type V systems, the RuvC-like nuclease domain of Cpf1 cleaves both strands of the target nucleic acid in a staggered configuration, producing 5′ overhangs, which is in contrast to the blunt ends generated by Cas9 cleavage. These 5′ overhangs may facilitate insertion of DNA through non-homologous end-joining (NHEJ) methods.

The Cpf1 cleavage activity of Type V systems also does not require hybridization of crRNA to tracrRNA to form a duplex, rather the crRNA of Type V systems use a single crRNA that has a stem loop structure forming an internal duplex. Cpf1 binds the crRNA in a sequence and structure specific manner, that recognizes the stem loop and sequences adjacent to the stem loop, most notably, the nucleotide 5′ of the spacer sequences that hybridizes to the target nucleic acid. This stem loop structure is typically in the range of 15 to 19 nucleotides in length. Substitutions that disrupt this stem loop duplex abolish cleavage activity, whereas other substitutions that do not disrupt the stem loop duplex do not abolish cleavage activity. In Type V systems, the crRNA forms a stem loop structure at the 5′ end and the sequence at the 3′ end is complementary to a sequence in a target nucleic acid.

Other proteins associated with Type V crRNA and target binding and cleavage include Class 2 candidate 1 (C2c1) and Class 2 candidate 3 (C2c3). C2c1 and C2c3 proteins are similar in length to Cas9 and Cpf1 proteins, ranging from approximately 1,100 amino acids to approximately 1,500 amino acids. C2c1 and C2c3 proteins also contain RuvC-like nuclease domains and have an architecture similar to Cpf1. C2c1 proteins are similar to Cas9 proteins in requiring a crRNA and a tracrRNA for target binding and cleavage, but have an optimal cleavage temperature of 50° C. C2c1 proteins target an AT-rich PAM, which similar to Cpf1, is 5′ of the target sequence, (Shmakov et al., Molecular Cell 60(3): 385-397 (2015)). Class 2 candidate 2 (C2c2) does not share sequence similarity to other CRISPR effector proteins, and therefore may be in a putative Type VI system. C2c2 proteins have two HEPN domains and are predicted to have RNase activity, and therefore may target and cleave mRNA. C2c2 proteins appear similar to Cpf1 proteins in requiring crRNA for target binding and cleavage, while not requiring tracrRNA. Also like Cpf1, the crRNA for C2c2 proteins forms a stable hairpin, or stem loop structure, that may aid in association with the C2c2 protein.

The gene editing systems provided herein may comprise Class 1 or Class 2 system components, including ribonucleic acid protein complexes. The Class 2 Cas nuclease families of proteins are enzymes with DNA endonuclease activity, and they can be directed to cleave a desired nucleic acid target by designing an appropriate guide RNA, as described further herein. A Class 2 CRISPR/Cas system component may be from a Type II, Type IIA, Type IIB, Type IIC, Type V, or Type VI system. Class 2 Cas nucleases include, for example, Cas9 (also known as Csn1 or Csx12), Csn2, Cas4, Cas12a (Cpf1), Cas12b (C2c1), Cas12c (C2c3), Cas13a (C2c2), Cas13b, Cas13c, and Cas13d proteins. In some embodiments, the Cas protein is from a Type II CRISPR/Cas system, i.e., a Cas9 protein from a CRISPR/Cas9 system, or a Type V CRISPR/Cas system, e.g., a Cas12a protein. In some embodiments, the Cas protein is from a Class 2 CRISPR/Cas system, i.e., a single-protein Cas nuclease such as a Cas9 protein or a Cas12a protein. Other non-limiting examples of Cas proteins can include Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, Cas9HiFi, homologues thereof, or modified versions thereof.

### Base Editing Base Editing System

Class 2 CRISPR/Cas systems, particularly CRISPR/Cas9 systems, have been used for precision genome editing. Through the use of a guide RNA (gRNA) with a sequence homologous to that of a sequence of DNA in the target genome (known as the protospacer) adjacent to a specific protospacer-adjacent motif (PAM) comprising the sequence NGG (N is any standard base) in the DNA, Cas9 can be used to create a double-strand break (DSB) at the targeted sequence. NHEJ at DSBs can be used to create indels and knock out genes at genetic loci; likewise, homology-directed repair (HDR) can be used, with an introduced template DNA, to insert genes or modify the targeted sequence.

In some aspects, provided herein are base editor systems capable of nucleobase modifications. In some embodiments, the base editor system comprises (i) a guide polynucleotide or a nucleic acid encoding same, and (ii) a base editor fusion protein comprising a programmable DNA binding domain (e.g., Cas9 or dCas9) and a deaminase, or a nucleic acid encoding same. In some embodiments, the base editor system comprises a guide polynucleotide. In some embodiments, the base editor system comprises a nucleic acid encoding a guide polynucleotide. In some embodiments, the base editor system comprises a base editor fusion protein comprising a programmable DNA binding domain and a deaminase. In some embodiments, the base editor system comprises a nucleic acid encoding a base editor fusion protein comprising a programmable DNA binding domain and a deaminase.

In some embodiments, the guide polynucleotide directs the base editor system to effect a nucleobase alteration in a PCSK9 or ANGPTL3 gene in vivo when administered to a subject.

In some embodiments, the base alteration occurs in at least 35% of whole liver cells in the subject as measured by next generation sequencing or Sanger sequencing.

In some embodiments, the base alteration occurs in at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% of whole liver cells in the subject as measured by next generation sequencing or Sanger sequencing. In some embodiments, the base alteration occurs in at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% of whole liver cells in the subject as measured by next generation sequencing or Sanger sequencing. In some embodiments, the base alteration occurs in 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 35%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% of whole liver cells in the subject as measured by next generation sequencing or Sanger sequencing. In some embodiments, the base alteration occurs in 1%-99.5%, 1%-99%, 1%-98%, 1%-97%, 1%-96%, 1%-95%, 1%-90%, 1%-85%, 1%-80%, 1%-75%, 1%-70%, 1%-65%, 1%-60%, 1%-55%, 1%-50%, 1%-45%, 1%-40%, 1%-35%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% of whole liver cells in the subject as measured by next generation sequencing or Sanger sequencing. In some embodiments, the base alteration occurs in 1%-90%, 5%-85%, 10%-80%, 15%-75%, 20%-70%, 25%-65%, 30%-60%, 35%-55%, or 40%-50% of whole liver cells in the subject as measured by next generation sequencing or Sanger sequencing. In some embodiments, the base alteration occurs in 100% of whole liver cells in the subject as measured by next generation sequencing or Sanger sequencing.

In some embodiments, the base alteration occurs in hepatocytes in the subject. In some embodiments, the base alteration occurs in at least 30% of hepatocytes in the subject as measured by next generation sequencing or Sanger sequencing. In some embodiments, the base alteration occurs in hepatocytes in the subject. In some embodiments, the base alteration occurs in at least % of hepatocytes in the subject as measured by next generation sequencing or Sanger sequencing. In some embodiments, the base alteration occurs in at least 1%, 2%, 3%, 4%, 5%, 6%, 7%, 8%, 9%, 10, 1%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% of hepatocytes in the subject as measured by next generation sequencing or Sanger sequencing. In some embodiments, the base alteration occurs in at most 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% of hepatocytes in the subject as measured by next generation sequencing or Sanger sequencing. In some embodiments, the base alteration occurs in 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 35%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% of hepatocytes in the subject as measured by next generation sequencing or Sanger sequencing. In some embodiments, the base alteration occurs in 1%-99.5%, 1%-99%, 1%-98%, 1%-97%, 1%-96%, 1%-95%, 1%-90%, 1%-85%, 1%-80%, 1%-75%, 1%-70%, 1%-65%, 1%-60%, 1%-55%, 1%-50%, 1%-45%, 1%-40%, 1%-35%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% hepatocytes in the subject as measured by next generation sequencing or Sanger sequencing. In some embodiments, the base alteration occurs in 1%-90%, 5%-85%, 10%-80%, 15%-75%, 20%-70%, 25%-65%, 30%-60%, 35%-55%, or 40%-50% of hepatocytes in the subject as measured by next generation sequencing or Sanger sequencing. In some embodiments, the base alteration occurs in 100% of hepatocytes in the subject as measured by next generation sequencing or Sanger sequencing.

In some embodiments, the base alteration occurred in whole liver cells in the subject is measured by next generation sequencing. In some embodiments, the base alteration occurred in whole liver cells in the subject is measured by Sanger sequencing. In some embodiments, the base alteration occurred in hepatocytes in the subject is measured by next generation sequencing. In some embodiments, the base alteration occurred in hepatocytes in the subject is measured by Sanger sequencing.

In some embodiments, the nucleobase alteration results in a reduction of at least 35% in blood PCSK9 protein level in the subject as compared to prior to the administration as measured by ELISA, Western blots, or LC-MS/MS. In some embodiments, the nucleobase alteration results in a reduction of at least 35% in blood ANGPTL3 protein level in the subject as compared to prior to the administration as measured by ELISA, Western blots, or LC-MS/MS.

In some embodiments, the nucleobase alteration results in a reduction of at least 30%, 35%, 40%, 45%, 50%, 55%, 60%, 65%, 70%, 75%, 80%, 85%, 90%, 95%, 97%, 98%, or 99% in blood PCSK9 protein level in the subject as compared to prior to the administration as measured by ELISA, Western blots, or LC-MS/MS. In some embodiments, the nucleobase alteration results in a reduction of at least 1%, 2%, 3%, 4%, 5%, 6%, 7%, 8%, 9%, 10%, 15%, 20%, 25%, 30%, 31%, 32%, 33%, 34%, 35%, 36%, 37%, 38%, 39%, 40%, 45%, 50%, 55%, 60%, 65%, 70%, 71%, 72%, 73%, 74%, 75%, 76%, 77%, 78%, 79%, 80%, 85%, 90%, 95%, 97%, 98%, 90%, 95%, 97%, 98%, 99%, 99.3%, 99.5%, 99.7%, 99.8%, or 99.9% in blood PCSK9 protein level in the subject as compared to prior to the administration as measured by ELISA, Western blots, or LC-MS/MS. In some embodiments, the nucleobase alteration results in a reduction of at most 1%, 2%, 3%, 4%, 5%, 6%, 7%, 8%, 9%, 10%, 15%, 20%, 25%, 30%, 31%, 32%, 33%, 34%, 35%, 36%, 37%, 38%, 39%, 40%, 45%, 50%, 55%, 60%, 65%, 70%, 71%, 72%, 73%, 74%, 75%, 76%, 77%, 78%, 79%, 80%, 85%, 90%, 95%, 97%, 98%, 90%, 95%, 97%, 98%, 99%, 99.3%, 99.5%, 99.7%, 99.8%, or 99.9% in blood PCSK9 protein level in the subject as compared to prior to the administration as measured by ELISA, Western blots, or LC-MS/MS. In some embodiments, the nucleobase alteration results in a reduction of 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 31%-99.9%, 32%-99.9%, 33%-99.9%, 34%-99.9%, 35%-99.9%, 36%-99.9%, 37%-99.9%, 38%-99.9%, 39%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% in blood PCSK9 protein level in the subject as compared to prior to the administration as measured by ELISA, Western blots, or LC-MS/MS. In some embodiments, the nucleobase alteration results in a reduction of 1%-99.5%, 1%-99%, 1%-98%, 1%-97%, 1%-96%, 1%-95%, 1%-90%, 1%-85%, 1%-80%, 1%-79%, 1%-78%, 1%-77%, 1%-76%, 1%-75%, 1%-74%, 1%-73%, 1%-72%, 1%-71%, 1%-70%, 1%-65%, 1%-60%, 1%-55%, 1%-50%, 1%-45%, 1%-40%, 1%-39%, 1%-38%, 1%-37%, 1%-36%, 1%-35%, 1%-34%, 1%-33%, 1%-32%, 1%-31%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% in blood PCSK9 protein level in the subject as compared to prior to the administration as measured by ELISA, Western blots, or LC-MS/MS. In some embodiments, the nucleobase alteration results in a reduction of 1%-99.9%, 5%-99.5%, 10%-99%, 15%-97%, 20%-95%, 25%-90%, 30%-85%, 31%-80%, 32%-79%, 33%-78%, 34%-77%, 35%-76%, 36%-76%, 37%-75%, 38%-74%, 39%-73%, 40%-72%, 45%-71%, 50%-70%, or 55%-65% in blood PCSK9 protein level in the subject as compared to prior to the administration as measured by ELISA, Western blots, or LC-MS/MS. In some embodiments, the nucleobase alteration results in a reduction of 100% in blood PCSK9 protein level in the subject as compared to prior to the administration as measured by ELISA, Western blots, or LC-MS/MS.

In some embodiments, the nucleobase alteration results in at least 1%, 2%, 3%, 4%, 5%, 6%, 7%, 8%, 9%, 10%, 11%, 12%, 13%, 14%, 15%, 20%, 25%, 30%, 35%, 40%, 50%, 75%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 400% 500%, 600%, 700%, 800%, 900%, 1000% less blood PCSK9 protein level in the subject as compared to prior to the administration as measured by ELISA, Western blots, or LC-MS/MS. In some embodiments, the nucleobase alteration results in at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, or more than 10-fold less blood PCSK9 protein level in the subject as compared to prior to the administration as measured by ELISA, Western blots, or LC-MS/MS.

In some embodiments, the reduction of blood PCSK9 protein level or the blood PCSK9 protein level in the subject as compared to prior to the administration is measured by ELISA (enzyme-linked immunosorbent assay). In some embodiments, the reduction of blood PCSK9 protein level or the blood PCSK9 protein level in the subject as compared to prior to the administration is measured by Western blot analysis. In some embodiments, the reduction of blood PCSK9 protein level or the blood PCSK9 protein level in the subject as compared to prior to the administration is measured by LC-MS/MS (liquid chromatography-tandem mass spectrometry).

In some embodiments, the nucleobase alteration results in a reduction of at least 30%, 35%, 40%, 45%, 50%, 55%, 60%, 65%, 70%, 75%, 80%, 85%, 90%, 95%, 97%, 98%, or 99% in blood ANGPTL3 protein level in the subject as compared to prior to the administration as measured by ELISA, Western blots, or LC-MS/MS. In some embodiments, the nucleobase alteration results in a reduction of at least 1%, 2%, 3%, 4%, 5%, 6%, 7%, 8%, 9%, 10%, 15%, 20%, 25%, 30%, 31%, 32%, 33%, 34%, 35%, 36%, 37%, 38%, 39%, 40%, 45%, 50%, 55%, 60%, 65%, 70%, 71%, 72%, 73%, 74%, 75%, 76%, 77%, 78%, 79%, 80%, 85%, 90%, 95%, 97%, 98%, 90%, 95%, 97%, 98%, 99%, 99.3%, 99.5%, 99.7%, 99.8%, or 99.9% in blood ANGPTL3 protein level in the subject as compared to prior to the administration as measured by ELISA, Western blots, or LC-MS/MS. In some embodiments, the nucleobase alteration results in a reduction of at most 1%, 2%, 3%, 4%, 5%, 6%, 7%, 8%, 9%, 10%, 15%, 20%, 25%, 30%, 31%, 32%, 33%, 34%, 35%, 36%, 37%, 38%, 39%, 40%, 45%, 50%, 55%, 60%, 65%, 70%, 71%, 72%, 73%, 74%, 75%, 76%, 77%, 78%, 79%, 80%, 85%, 90%, 95%, 97%, 98%, 90%, 95%, 97%, 98%, 99%, 99.3%, 99.5%, 99.7%, 99.8%, or 99.9% in blood ANGPTL3 protein level in the subject as compared to prior to the administration as measured by ELISA, Western blots, or LC-MS/MS. In some embodiments, the nucleobase alteration results in a reduction of 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 31%-99.9%, 32%-99.9%, 33%-99.9%, 34%-99.9%, 35%-99.9%, 36%-99.9%, 37%-99.9%, 38%-99.9%, 39%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% in blood ANGPTL3 protein level in the subject as compared to prior to the administration as measured by ELISA, Western blots, or LC-MS/MS. In some embodiments, the nucleobase alteration results in a reduction of 1%-99.5%, 1%-99%, 1%-98%, 1%-97%, 1%-96%, 1%-95%, 1%-90%, 1%-85%, 1%-80%, 1%-79%, 1%-78%, 1%-77%, 1%-76%, 1%-75%, 1%-74%, 1%-73%, 1%-72%, 1%-71%, 1%-70%, 1%-65%, 1%-60%, 1%-55%, 1%-50%, 1%-45%, 1%-40%, 1%-39%, 1%-38%, 1%-37%, 1%-36%, 1%-35%, 1%-34%, 1%-33%, 1%-32%, 1%-31%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% in blood ANGPTL3 protein level in the subject as compared to prior to the administration as measured by ELISA, Western blots, or LC-MS/MS. In some embodiments, the nucleobase alteration results in a reduction of 1%-99.9%, 5%-99.5%, 10%-99%, 15%-97%, 20%-95%, 25%-90%, 30%-85%, 31%-80%, 32%-79%, 33%-78%, 34%-77%, 35%-76%, 36%-76%, 37%-75%, 38%-74%, 39%-73%, 40%-72%, 45%-71%, 50%-70%, or 55%-65% in blood ANGPTL3 protein level in the subject as compared to prior to the administration as measured by ELISA, Western blots, or LC-MS/MS. In some embodiments, the nucleobase alteration results in a reduction of 100% in blood ANGPTL3 protein level in the subject as compared to prior to the administration as measured by ELISA, Western blots, or LC-MS/MS.

In some embodiments, the nucleobase alteration results in at least 1%, 2%, 3%, 4%, 5%, 6%, 7%, 8%, 9%, 10%, 11%, 12%, 13%, 14%, 15%, 20%, 25%, 30%, 35%, 40%, 50%, 75%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 400% 500%, 600%, 700%, 800%, 900%, 1000% less blood ANGPTL3 protein level in the subject as compared to prior to the administration as measured by ELISA, Western blots, or LC-MS/MS. In some embodiments, the nucleobase alteration results in at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, or more than 10-fold less blood ANGPTL3 protein level in the subject as compared to prior to the administration as measured by ELISA, Western blots, or LC-MS/MS.

In some embodiments, the reduction of blood ANGPTL3 protein level or the blood ANGPTL3 protein level in the subject as compared to prior to the administration is measured by ELISA (enzyme-linked immunosorbent assay). In some embodiments, the reduction of blood ANGPTL3 protein level or the blood ANGPTL3 protein level in the subject as compared to prior to the administration is measured by Western blot analysis. In some embodiments, the reduction of blood ANGPTL3 protein level or the blood ANGPTL3 protein level in the subject as compared to prior to the administration is measured by LC-MS/MS (liquid chromatography-tandem mass spectrometry).

In some embodiments, the nucleobase alteration results in a reduction of at least 35% in blood or low-density lipoprotein cholesterol (LDL-C) levels in the subject as compared to prior to the administration. In some embodiments, the nucleobase alteration results in a reduction of at least 35%, 40%, 45%, 46%, 47%, 48%, 49%, 50%, 51%, 52%, 53%, 54%, 55%, 56%, 57%, 58%, 59%, 60%, 61%, 62%, 63%, 64%, 65%, 70%, 75%, 80%, 85%, 90%, 95%, 97%, 98%, or 99% in blood low-density lipoprotein cholesterol (LDL-C) level in the subject as compared to prior to the administration. In some embodiments, the nucleobase alteration results in a reduction of at least 1%, 2%, 3%, 4%, 5%, 6%, 7%, 8%, 9%, 10%, 15%, 20%, 25%, 30%, 35%, 40%, 45%, 46%, 47%, 48%, 49%, 50%, 51%, 52%, 53%, 54%, 55%, 56%, 57%, 58%, 59%, 60%, 61%, 62%, 63%, 64%, 65%, 70%, 75%, 80%, 85%, 90%, 95%, 97%, 98%, 99%, 99.3%, 99.5%, 99.7%, 99.8%, or 99.9% in blood low-density lipoprotein cholesterol (LDL-C) level in the subject as compared to prior to the administration. In some embodiments, the nucleobase alteration results in a reduction of at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% in blood low-density lipoprotein cholesterol (LDL-C) level in the subject as compared to prior to the administration. In some embodiments, the nucleobase alteration results in a reduction of 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 35%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% in blood low-density lipoprotein cholesterol (LDL-C) level in the subject as compared to prior to the administration. In some embodiments, the nucleobase alteration results in a reduction of 1%-99.5%, 1%-99%, 1%-98%, 1%-97%, 1%-96%, 1%-95%, 1%-90%, 1%-85%, 1%-80%, 1%-75%, 1%-70%, 1%-65%, 1%-60%, 1%-55%, 1%-50%, 1%-45%, 1%-40%, 1%-35%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% in blood low-density lipoprotein cholesterol (LDL-C) level in the subject as compared to prior to the administration. In some embodiments, the nucleobase alteration results in a reduction of 1%-99.9%, 5%-99.5%, 10%-99%, 15%-97%, 20%-95%, 25%-90%, 30%-85%, 35%-80%, 40%-75%, 45%-70%, 50%-65%, or 55%-60% in blood low-density lipoprotein cholesterol (LDL-C) level in the subject as compared to prior to the administration. In some embodiments, the nucleobase alteration results in a reduction of 100% in blood low-density lipoprotein cholesterol (LDL-C) level in the subject as compared to prior to the administration. In some embodiments, the nucleobase alteration results in at least 1%, 2%, 3%, 4%, 5%, 6%, 7%, 8%, 9%, 10%, 11%, 12%, 13%, 14%, 15%, 20%, 25%, 30%, 35%, 40%, 50%, 75%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 400% 500%, 600%, 700%, 800%, 900%, 1000% less blood low-density lipoprotein cholesterol (LDL-C) level in the subject as compared to prior to the administration. In some embodiments, the nucleobase alteration results in at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, or more than 10-fold less blood low-density lipoprotein cholesterol (LDL-C) level in the subject as compared to prior to the administration.

In some embodiments, the nucleobase alteration results in a reduction of at least 35% in blood triglyceride levels in the subject as compared to prior to the administration. In some embodiments, the nucleobase alteration results in a reduction of at least 30%, 35%, 40%, 45%, 46%, 47%, 48%, 49%, 50%, 51%, 52%, 53%, 54%, 55%, 56%, 57%, 58%, 59%, 60%, 61%, 62%, 63%, 64%, 65%, 70%, 75%, 80%, 85%, 90%, 95%, 97%, 98%, or 99% in blood triglyceride level in the subject as compared to prior to the administration. In some embodiments, the nucleobase alteration results in a reduction of at least 1%, 2%, 3%, 4%, 5%, 6%, 7%, 8%, 9%, 10%, 15%, 20%, 25%, 30%, 35%, 40%, 45%, 46%, 47%, 48%, 49%, 50%, 51%, 52%, 53%, 54%, 55%, 56%, 57%, 58%, 59%, 60%, 61%, 62%, 63%, 64%, 65%, 70%, 75%, 80%, 85%, 90%, 95%, 97%, 98%, 99%, 99.3%, 99.5%, 99.7%, 99.8%, or 99.9% in blood triglyceride level in the subject as compared to prior to the administration. In some embodiments, the nucleobase alteration results in a reduction of at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, 99.3%, 99.5%, 99.7%, 99.8%, or 99.9% in blood triglyceride level in the subject as compared to prior to the administration. In some embodiments, the nucleobase alteration results in a reduction of 1%-99.9%, 2%-99.9%, 3%-99.9%, 4%-99.9%, 5%-99.9%, 6%-99.9%, 7%-99.9%, 8%-99.9%, 9%-99.9%, 10%-99.9%, 15%-99.9%, 20%-99.9%, 25%-99.9%, 30%-99.9%, 35%-99.9%, 40%-99.9%, 45%-99.9%, 50%-99.9%, 55%-99.9%, 60%-99.9%, 65%-99.9%, 70%-99.9%, 75%-99.9%, 80%-99.9%, 85%-99.9%, 90%-99.9%, or 95-99.9% in blood triglyceride level in the subject as compared to prior to the administration. In some embodiments, the nucleobase alteration results in a reduction of 1%-99.5%, 1%-99%, 1%-98%, 1%-97%, 1%-96%, 1%-95%, 1%-90%, 1%-85%, 1%-80%, 1%-75%, 1%-70%, 1%-65%, 1%-60%, 1%-55%, 1%-50%, 1%-45%, 1%-40%, 1%-35%, 1%-30%, 1%-25%, 1%-20%, 1%-15%, 1%-10%, 1%-9%, 1%-8%, 1%-7%, 1%-6%, 1%-5%, 1%-4%, 1%-3%, or 1%-2% in blood triglyceride level in the subject as compared to prior to the administration. In some embodiments, the nucleobase alteration results in a reduction of 1%-99.9%, 5%-99.5%, 10%-99%, 15%-97%, 20%-95%, 25%-90%, 30%-85%, 35%-80%, 40%-75%, 45%-70%, 50%-65%, or 55%-60% in blood triglyceride level in the subject as compared to prior to the administration. In some embodiments, the nucleobase alteration results in a reduction of 100% in blood triglyceride level in the subject as compared to prior to the administration. In some embodiments, the nucleobase alteration results in at least 1%, 2%, 3%, 4%, 5%, 6%, 7%, 8%, 9%, 10%, 11%, 12%, 13%, 14%, 15%, 20%, 25%, 30%, 35%, 40%, 50%, 75%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 400% 500%, 600%, 700%, 800%, 900%, 1000% less blood triglyceride level in the subject as compared to prior to the administration. In some embodiments, the nucleobase alteration results in at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, or more than 10-fold less blood triglyceride level in the subject as compared to prior to the administration.

In some embodiments, the blood triglyceride level or the reduction of blood triglyceride level in the subject as compared to prior to the administration is measured by any standard technique. In some embodiments, the blood low-density lipoprotein cholesterol (LDL-C) level or the reduction of blood low-density lipoprotein cholesterol (LDL-C) level in the subject as compared to prior to the administration is measured by any standard technique. For example, a clinical analyzer instrument may be used to measure a ‘lipid panel’ in serum samples which entails the direct measurement of cholesterol (total C), triglycerides (TG) and high-density lipoprotein cholesterol (HDL-C) enzymatically. Reagent kits specific for each analyte contain buffers, calibrators, blanks and controls. As used in the present disclosure, cholesterol, triglycerides and HDL-C may be quantified using absorbance measurements of specific enzymatic reaction products. LDL-C may be determined indirectly. In some instances, most of circulating cholesterol can be found in three major lipoprotein fractions: very low-density lipoproteins (VLDL), LDL and HDL. In some embodiments, total circulating cholesterol may be estimated with the formula [Total C]=[VLDL-C]+[LDL-C]+[HDL-C]. Thus the LDL-C can be calculated from measured values of total cholesterol, triglycerides and HDL-C according to the relationship: [LDL-C]=[total C]−[HDL-C]−[TG]/5, where [TG]/5 is an estimate of VLDL-cholesterol. A reagent kit specific for triglycerides containing buffers, calibrators, blanks and controls may be used. As used herein, serum samples from the study may be analyzed and triglycerides may be measured using a series of coupled enzymatic reactions. In some embodiments, H₂O₂ may be used to quantify the analyte. as the end product of the last one and its absorbance at 500 nm, and the color intensity is proportional to triglyceride concentrations.

In some embodiments, the guide polynucleotide is a guide RNA, wherein the guide RNA comprises a spacer sequence that binds to the complementary strand of a protospacer sequence of the PCSK9 gene with 0, 1, or 2 mismatches. In some embodiments, the guide RNA comprises a spacer sequence that binds to the complementary strand of a protospacer sequence of the PCSK9 gene with no mismatches. In some embodiments, the guide RNA comprises a spacer sequence that binds to the complementary strand of a protospacer sequence of the PCSK9 gene with 1 mismatch. In some embodiments, the guide RNA comprises a spacer sequence that binds to the complementary strand of a protospacer sequence of the PCSK9 gene with 2 mismatches. In some embodiments, the guide RNA comprises a spacer sequence that binds to the complementary strand of a protospacer sequence of the PCSK9 gene with 3 mismatches. In some embodiments, the guide RNA comprises a spacer sequence that binds to the complementary strand of a protospacer sequence of the PCSK9 gene with 4 mismatches. In some embodiments, the guide RNA comprises a spacer sequence that binds to the complementary strand of a protospacer sequence of the PCSK9 gene with 5 mismatches.

In some embodiments, the guide polynucleotide is a guide RNA, wherein the guide RNA comprises a spacer sequence that binds to the complementary strand of a protospacer sequence of the ANGPTL3 gene with 0, 1, or 2 mismatches. In some embodiments, the guide RNA comprises a spacer sequence that binds to the complementary strand of a protospacer sequence of the ANGPTL3 gene with no mismatches. In some embodiments, the guide RNA comprises a spacer sequence that binds to the complementary strand of a protospacer sequence of the ANGPTL3 gene with 1 mismatch. In some embodiments, the guide RNA comprises a spacer sequence that binds to the complementary strand of a protospacer sequence of the ANGPTL3 gene with 2 mismatches. In some embodiments, the guide RNA comprises a spacer sequence that binds to the complementary strand of a protospacer sequence of the ANGPTL3 gene with 3 mismatches. In some embodiments, the guide RNA comprises a spacer sequence that binds to the complementary strand of a protospacer sequence of the ANGPTL3 gene with 4 mismatches. In some embodiments, the guide RNA comprises a spacer sequence that binds to the complementary strand of a protospacer sequence of the ANGPTL3 gene with 5 mismatches.

In some embodiments, the nucleobase alteration is outside of the protospacer sequence in less than 1% of whole liver cells in the subject as measured by net nucleobase editing. In some embodiments, the nucleobase alteration is outside of the protospacer sequence in less than 1% of hepatocytes in the subject as measured by net nucleobase editing. In some embodiments, the nucleobase alteration is only within the protospacer sequence as measured by net nucleobase editing.

In some embodiments, the nucleobase alteration is outside of the protospacer sequence in less than 0.01%. 0.02%, 0.03% 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0% 4.0%, 5.0%, 6.0%, 7.0%, 8.0%, 9.0%, 10%, 15%, 20%, 25%, 30%, 35%, 40%, 45%, 50%, 55%, 60%, 65%, 70%, 65%, 80%, 85%, 90% of whole liver cells in the subject as measured by net nucleobase editing. In some embodiments, the nucleobase alteration is outside of the protospacer sequence in less than 0.01%. 0.02%, 0.03% 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0% 4.0%, 5.0%, 6.0%, 7.0%, 8.0%, 9.0%, 10%, 15%, 20%, 25%, 30%, 35%, 40%, 45%, 50%, 55%, 60%, 65%, 70%, 65%, 80%, 85%, 90% of hepatocytes in the subject as measured by net nucleobase editing. In some embodiments, the nucleobase alteration is outside of the protospacer sequence in less than 0.01%. 0.02%, 0.03% 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0% 4.0%, 5.0%, 6.0%, 7.0%, 8.0%, 9.0%, 10, 1%, 20%, 25%, 30%, 35%, 40%, 45%, 50%, 55%, 60%, 65%, 70%, 65%, 80%, 85%, 90% of cells in the subject as measured by net nucleobase editing.

In some embodiments, the deaminase is an adenine deaminase. In some embodiments, the nucleobase alteration is a A●T to G●C alteration. In some embodiments, the deaminase is an adenine deaminase and the nucleobase alteration is a A●T to G●C alteration. In some embodiments, the programmable DNA binding domain comprises a nuclease inactive Cas9 or a Cas9 nickase. In some embodiments, the programmable DNA binding domain comprises a Cas9.

In some embodiments, the nucleobase alteration is at a splice site of the PCSK9 gene. In some embodiments, the nucleobase alteration is at a splice donor site of the PCSK9 gene. In some embodiments, the splice donor site is at 5′ end of PCSK9 intron 1 as referenced in SEQ ID NO: 5. In some embodiments, the nucleobase alteration is at a splice acceptor site of the PCSK9 gene. In some embodiments, the nucleobase alteration results in a frame shift, a premature stop codon, a insertion or deletion in a transcript encoded by the PCSK9 gene. In some embodiments, the nucleobase alteration results in an aberrant transcript encoded by the PCSK9 gene. In some embodiments, the guide polynucleotide is a guide RNA. In some embodiments, the guide RNA is chemically modified. In some embodiments, the guide RNA comprises a tracrRNA sequence. In some embodiments, the guide RNA comprises a chemical modification. Exemplary guide RNA comprising a chemical modification can be found in PCT Application Publication No. WO2021/207712, which is hereby incorporated by reference in its entirety.

In some embodiments, the nucleobase alteration is at a splice site of the ANGPTL3 gene. In some embodiments, the nucleobase alteration is at a splice donor site of the ANGPTL3 gene. In some embodiments, the splice donor site is at 5′ end of ANGPTL3 intron 6 as referenced in SEQ ID NO: 1. In some embodiments, the nucleobase alteration is at a splice acceptor site of the ANGPTL3 gene. In some embodiments, the nucleobase alteration results in a frame shift, a premature stop codon, an insertion or deletion in a transcript encoded by the ANGPTL3 gene. In some embodiments, the nucleobase alteration results in an aberrant transcript encoded by the ANGPTL3 gene. In some embodiments, the guide polynucleotide is a guide RNA. In some embodiments, the guide RNA is chemically modified. In some embodiments, the guide RNA comprises a tracrRNA sequence. In some embodiments, the guide RNA comprises a chemical modification. Exemplary guide RNA comprising a chemical modification can be found in PCT Application Publication No. WO2021/207712, which is hereby incorporated by reference in its entirety.

In some embodiments, the guide RNA comprises a guide RNA sequence. Exemplary additional guide RNA sequence can be found in PCT Application Publication No. WO2021/207712, which is hereby incorporated by reference in its entirety.

In some embodiments, the protospacer sequence comprises a protospacer sequence. In some embodiments, the protospacer comprises the sequence 5′-CCCGCACCTTGGCGCAGCGG-3′ (SEQ ID NO: 731), AAGATACCTGAATAACTCTC-3′ (SEQ ID NO: 732), and 5′-AAGATACCTGAATAACCCTC-3′ (SEQ ID NO: 733).

In some embodiments, the base editor fusion protein comprises the sequence of SEQ ID NO: 734. In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid sequence set forth in SEQ ID NO: 734 or to any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to the amino acid sequence set forth in SEQ ID NO: 734 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NO: 734 or any of the adenosine deaminases provided herein.

In some embodiments, the nucleic acid encoding the base editor fusion protein is a mRNA. The mRNA may comprise modifications, for example, modifications at 3′ or 5′ end of the mRNA. In some embodiments, the mRNA comprises a cap analog.

In some embodiments, the mRNA comprises at least 1, 2, or 3 nucleotides at the 5′ end that comprises 2′-hydroxyl group, 2′-O-methyl group, or additional 2′ chemical modification or a combination thereof. In some embodiments, the mRNA comprises at least 1, 2, or 3 nucleotides at the 5′ end that comprises 2′-hydroxyl group, 2′-O-methyl group, or additional 2′ chemical modification or a combination thereof. In some embodiments, the mRNA comprises at least 1 nucleotide at the 5′ end that comprises 2′-hydroxyl group, 2′-O-methyl group, or additional 2′ chemical modification or a combination thereof. In some embodiments, the mRNA comprises at least 2 nucleotides at the 5′ end that comprises 2′-hydroxyl group, 2′-O-methyl group, or additional 2′ chemical modification or a combination thereof. In some embodiments, the mRNA comprises at least 3 nucleotides at the 5′ end that comprises 2′-hydroxyl group, 2′-O-methyl group, or additional 2′ chemical modification or a combination thereof. In some embodiments, the mRNA comprises at least 4 nucleotides at the 5′ end that comprises 2′-hydroxyl group, 2′-O-methyl group, or additional 2′ chemical modification or a combination thereof. In some embodiments, the mRNA comprises at least 5 nucleotides at the 5′ end that comprises 2′-hydroxyl group, 2′-O-methyl group, or additional 2′ chemical modification or a combination thereof. In some embodiments, the mRNA comprises at least 6 nucleotides at the 5′ end that comprises 2′-hydroxyl group, 2′-O-methyl group, or additional 2′ chemical modification or a combination thereof. In some embodiments, the mRNA comprises at least 7 nucleotides at the 5′ end that comprises 2′-hydroxyl group, 2′-O-methyl group, or additional 2′ chemical modification or a combination thereof. In some embodiments, the mRNA comprises at least 8 nucleotides at the 5′ end that comprises 2′-hydroxyl group, 2′-O-methyl group, or additional 2′ chemical modification or a combination thereof. In some embodiments, the mRNA comprises at least 9 nucleotides at the 5′ end that comprises 2′-hydroxyl group, 2′-O-methyl group, or additional 2′ chemical modification or a combination thereof. In some embodiments, the mRNA comprises at least 10 nucleotides at the 5′ end that comprises 2′-hydroxyl group, 2′-O-methyl group, or additional 2′ chemical modification or a combination thereof.

In some embodiments, the mRNA comprises a poly A tail. The poly A tail may be at the 3′ end of the mRNA.

In some embodiments, the GC % content of the mRNA sequence is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the mRNA sequence is greater than or equal to 40%, 45%, 50%, 55%, 60%, 65%, 70%, 75% or 80%.

In some embodiments, the mRNA sequence comprises an adenine tTNA deaminase (TadA) region. In some embodiments, the GC % of the TadA region is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the TadA region is greater than or equal to 40%, 45%, 50%, 55%, 60%, 65%, 70%, 75% or 80%.

In some embodiments, the mRNA sequence comprises a Cas9 region. In some embodiments, the GC % of the Cas9 region is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the Cas9 region is greater than or equal to 40%, 45%, 50%, 55%, 60%, 65%, 70%, 75% or 80%.

In some embodiments, the mRNA sequence comprises a NLS region. In some embodiments, the GC % of the NLS region is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the NLS region is greater than or equal to 40%, 45%, 50%, 55%, 60%, 65%, 70%, 75% or 80%.

In some embodiments, the mRNA sequence comprises a first linker region that connects the TadA region and the Cas9 region. In some embodiments, the GC % of the first linker region is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the first linker region is greater than or equal to 40%, 45%, 50%, 55%, 60%, 65%, 70%, 75% or 80%.

In some embodiments, the mRNA sequence comprises a second linker region that connects the Cas9 region and the NLS region. In some embodiments, the GC % of the second linker region is greater than or equal to 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the GC % content of the second linker region is greater than or equal to 40%, 45%, 50%, 55%, 60%, 65%, 70%, 75% or 80%.

In some embodiments, the base editor system as provided herein further comprises a lipid nanoparticle (LNP) enclosing a guide polynucleotide or a nucleic acid encoding the guide polynucleotide (i). In some embodiments, the LNP further encloses a base editor fusion protein comprising a programmable DNA binding domain and a deaminase, or a nucleic acid encoding same (ii). In some embodiments, the base editor system further comprises a second LNP enclosing a base editor fusion protein comprising a programmable DNA binding domain and a deaminase, or a nucleic acid encoding same (ii).

A base editor system as provided herein can include one or more LNPs. For example, a base editor system may comprise a LNP enclosing both a guide polynucleotide and a nucleic acid encoding the base editor fusion protein, e.g. an mRNA encoding the base editor fusion protein. In another example, a base editor system may comprise a LNP enclosing a guide polynucleotide, e.g. a guide RNA, and a LNP enclosing a nucleic acid, e.g. an mRNA, encoding the base editor fusion protein. LNPs separately enclosing the guide polynucleotide and the base editor fusion protein or mRNA encoding the base editor fusion protein may allow for flexible dosing and administration of the base editor system. For example, a LNP enclosing a guide RNA can be administered first, followed by administration of a LNP enclosing a mRNA encoding the base editor fusion protein. In some embodiments, a LNP enclosing a guide RNA and a second LNP enclosing a mRNA encoding the base editor fusion protein are administered to a subject at the same time. In some embodiments, a LNP enclosing a guide RNA and a LNP enclosing a mRNA encoding the base editor protein are administered to a subject sequentially. In some embodiments, a LNP enclosing mRNA encoding the base editor fusion protein is administered to a subject, followed by multiple administration or doses of a second LNP enclosing a guide RNA after 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, or 12 weeks or more. The multiple doses of the second LNP may be administered with intervals of 1, 2, 3, 4, 5, 6, 7, 8, 9, 10, 11, 12, 13, 14, 15, 16, 17, 18, 19, or 20 days or more.

In some embodiments, the ratio of the guide polynucleotide and the nucleic acid encoding the base editor fusion protein is about 1:10 to about 10:1 by weight. In some embodiments, the ratio of the guide polynucleotide and the nucleic acid encoding the base editor fusion protein is about 1:1, 1.5:1, 2:1, 3:1, 4:1, 1:1.5, 1:2, 1:3, 1:4 or any ratio between 4:1 or 1:4 by weight. In some embodiments, the ratio of the guide polynucleotide and the nucleic acid encoding the base editor fusion protein can be determined by titration of the guide polynucleotide and the nucleic acid encoding the base editor fusion protein.

In some embodiments, the molar ratio of the guide polynucleotide and the nucleic acid encoding the base editor fusion protein is about 500:1 to about 1:500.

In some embodiments, the ratio of the guide polynucleotide and the nucleic acid encoding the base editor fusion protein is about 1000:1 to about 1:1000 by weight. In some embodiments, the ratio of the guide polynucleotide and the nucleic acid encoding the base editor fusion protein is about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of the guide polynucleotide and the nucleic acid encoding the base editor fusion protein is about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight. In some embodiments, the ratio of the guide polynucleotide and the nucleic acid encoding the base editor fusion protein is at least about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of the guide polynucleotide and the nucleic acid encoding the base editor fusion protein is at least about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight. In some embodiments, the ratio of the guide polynucleotide and the nucleic acid encoding the base editor fusion protein is at most about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of the guide polynucleotide and the nucleic acid encoding the base editor fusion protein is at most about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight.

In some embodiments, the molar ratio of the guide polynucleotide and the nucleic acid encoding the base editor fusion protein is about 10000:1 to about 1:10000. In some embodiments, the molar ratio of the guide polynucleotide and the nucleic acid encoding the base editor fusion protein is about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of the guide polynucleotide and the nucleic acid encoding the base editor fusion protein is at least about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1. In some embodiments, the molar ratio of the guide polynucleotide and the nucleic acid encoding the base editor fusion protein is at least about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of the guide polynucleotide and the nucleic acid encoding the base editor fusion protein is at least about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1. In some embodiments, the molar ratio of the guide polynucleotide and the nucleic acid encoding the base editor fusion protein is at most about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of the guide polynucleotide and the nucleic acid encoding the base editor fusion protein is at most about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.

In some embodiments, the ratio of a nucleic acid encoding the guide polynucleotide and the nucleic acid encoding the base editor fusion protein is about 10:1 to about 1:10 by weight. In some embodiments, the ratio of a nucleic acid encoding the guide polynucleotide and the nucleic acid encoding the base editor fusion protein is about 4:1, 3;1, 2:1, 1.5:1, 1:1, 1:1.5, 1:2, 1:3, or 1:4 by weight. In some embodiments, the molar ratio of a nucleic acid encoding the guide polynucleotide and the nucleic acid encoding the base editor fusion protein is about 500:1 to about 1:500.

In some embodiments, the ratio of a nucleic acid encoding the guide polynucleotide and the nucleic acid encoding the base editor fusion protein is about 1000:1 to about 1:1000 by weight. In some embodiments, the ratio of a nucleic acid encoding the guide polynucleotide and the nucleic acid encoding the base editor fusion protein is about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of a nucleic acid encoding the guide polynucleotide and the nucleic acid encoding the base editor fusion protein is about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight. In some embodiments, the ratio of a nucleic acid encoding the guide polynucleotide and the nucleic acid encoding the base editor fusion protein is at least about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of a nucleic acid encoding the guide polynucleotide and the nucleic acid encoding the base editor fusion protein is at least about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight. In some embodiments, the ratio of a nucleic acid encoding the guide polynucleotide and the nucleic acid encoding the base editor fusion protein is at most about 1000:1, 950:1, 900:1, 850:1, 800:1, 750:1, 700:1, 650:1, 600:1, 550:1, 500:1, 450:1, 400:1, 350:1, 300:1, 250:1, 200:1, 100:1, 95:1, 90:1, 85:1, 80:1, 75:1, 70:1, 65:1, 60:1, 55:1, 50:1, 45:1, 40:1, 35:1, 30:1, 25:1, 20:1, 19:1, 18:1, 17:1, 17:1, 15:1, 14:1, 13:1, 12:1, 11:1, 10:1, 9:1, 8:1, 7:1, 6:1, 5:1, 4:1, 3:1, 2;1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, 0.5:1, 0.4:1, 0.3:1, 0.2:1, or 0.1 by weight. In some embodiments, the ratio of a nucleic acid encoding the guide polynucleotide and the nucleic acid encoding the base editor fusion protein is at most about 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000 by weight.

In some embodiments, the molar ratio of a nucleic acid encoding the guide polynucleotide and the nucleic acid encoding the base editor fusion protein is about 10000:1 to about 1:10000. In some embodiments, the molar ratio of a nucleic acid encoding the guide polynucleotide and the nucleic acid encoding the base editor fusion protein is about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of a nucleic acid encoding the guide polynucleotide and the nucleic acid encoding the base editor fusion protein is at least about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1. In some embodiments, the molar ratio of a nucleic acid encoding the guide polynucleotide and the nucleic acid encoding the base editor fusion protein is at least about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of a nucleic acid encoding the guide polynucleotide and the nucleic acid encoding the base editor fusion protein is at least about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1. In some embodiments, the molar ratio of a nucleic acid encoding the guide polynucleotide and the nucleic acid encoding the base editor fusion protein is at most about 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 190:1, 180:1, 170:1, 160:1, 150:1, 140:1, 130:1, 120:1, 110:1, 100:1, 95:1, 90:1, 85:1, 80:1, 75:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, or 0.1:1. In some embodiments, the molar ratio of a nucleic acid encoding the guide polynucleotide and the nucleic acid encoding the base editor fusion protein is at most about 1:10000, 1:9500, 1:9000, 1:8500, 1:8000, 1:7500, 1:7000, 1:6500, 1:6000, 1:5500, 1:5000, 1:4500, 1:4000, 1:3500, 1:3000, 1:2500, 1:2000, 1:1500, 1:1000, 1:950, 1:900, 1:850, 1:800, 1:750, 1:700, 1:650, 1:600, 1:550, 1:500, 1:450, 1:400, 1:350, 1:300, 1:250, 1:200, 1:190, 1:180, 1:170, 1:160, 1:150, 1:140, 1:130, 1:120, 1:110, 1:100, 1:95, 1:90, 1:85, 1:80, 1:75, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.

Precision genome editing is a growing field with industrial, agricultural, and biomedical applications. One of the dominant genome-editing systems available today is clustered regularly interspaced short palindromic repeats (CRISPR)—CRISPR associated 9 (Cas9). Through the use of a guide RNA (gRNA) with a sequence homologous to that of a sequence of DNA in the target genome (known as the protospacer) adjacent to a specific protospacer-adjacent motif (PAM) comprising the sequence NGG (N is any standard base) in the DNA, Cas9 can be used to create a double-strand break (DSB) at the targeted sequence. Non-homologous end joining (NHEJ) at DSBs can be used to create indels and knock out genes at genetic loci; likewise, homology-directed repair (HDR) can be used, with an introduced template DNA, to insert genes or modify the targeted sequence. A variety of Cas9-based tools have been developed in recent years, including tools that methylate DNA, recognize broader sequence space, or create single-strand nicks. In 2016, Komor et al. described the use of CRISPR-Cas9 to convert a cytosine base to a thymine base without the introduction of a template DNA strand and without the need for DSBs (Komor A C, Kim Y B, Packer M S, et al. Programmable editing of a target base in genomic DNA without double-stranded DNA cleavage. Nature, 2016, 533: 420-4, incorporated herein by reference in its entirety). After the cytidine deaminase domain of rat APOBEC1 was fused to the N-terminus of catalytically-dead Cas9 (dCas9) using the linker XTEN (resulting in a fusion protein called base editor 1, or BET), conversion of cytosine to uracil was observed between position 4 and position 8 within the 20-nt protospacer region of DNA (or, to express it a different way, 13 to 17 nucleotides upstream of the PAM). Of note, any cytosine base within this “window” was amenable to editing, resulting in varied outcomes depending on how many and which cytosines were edited. After DNA replication or repair, each uracil was replaced by a thymine, completing the C to T base editing.3 The next version of base editor (BE2) incorporated a uracil glycosylase inhibitor fused to the C-terminus of dCas9 to help inhibit base excision repair of the uracil bases resulting from the cytidine deaminase activity (which otherwise would act to restore the original cytosine bases); this improved the efficiency of C to T base editing. The final version, BE3, used a Cas9 nickase rather than dCas9; the nickase cut the unedited strand opposite the edited C to T bases, stimulating the removal of the opposing guanidine through eukaryotic mismatch repair. BE2 and BE3 base editing was observed in both human and murine cell lines. The specificity of base editing has been further improved through the addition of mutations to the Cas9 nickase; in similar fashion, Cas9 has been mutated to narrow the width of the editing window from approximately 5 nucleotides to as little as 1-2 nucleotides (Rees H A, Komor A C, Yeh W H, et al. Improving the DNA specificity and applicability of base editing through protein engineering and protein delivery. Nat Commun, 2017, 8: 15790, Kim Y B, Komor A C, Levy J M, et al. Increasing the genome-targeting scope and precision of base editing with engineered Cas9-cytidine deaminase fusions. Nat Biotechnol, 2017, 35: 371-6, each of which is incorporated herein by reference in its entirety).

An alternative cytosine base editing platform is by linking the activation-induced cytosine deaminase domain PmCDAT to dCas9 (Target-AID), they were able to demonstrate targeted C to T base editing in yeast. Furthermore, an alternative C to T editing strategy was also demonstrated without fusing a deaminase domain to Cas9; instead, a SH3 (Src 3 homology) domain was added to the C-terminus of dCas9 while a SHL (SH3 interaction ligand) was added to PmCDA1.6 Optimization of efficiency was achieved through the use of a Cas9 nickase rather than dCas9. Further, an uracil DNA glycosylase inhibitor was added to enhance base editing in the mammalian CHO cell line. The resulting platform was able to consistently edit bases within 3 to 5 bases of the 18th nucleotide upstream of the PAM sequence (Nishida K et al., Science, 2016, 353: aaf8729).

A distinct cytosine base editing platform used Cpf1 (also known as Cas12a) instead of Cas9 as the RNA-guided endonuclease. Catalytically-inactive Cpf1 was fused to APOBEC1 (dLbCpf1-BE0), leading to C to T conversion in a human cell line. While the Cas9 base editor variants BE3 and Target-AID recognize the PAM sequence NGG, dLbCpf1-BE0 recognizes the T-rich PAM sequence TTTV. Although base editing was observed between positions 8 and 13 of the protospacer sequence with dLbCpf1-BE0, the introduction of additional mutations into Cpf1 was able to reduce the window to positions 10 to 12. However, narrowing of the base editing window correlated with a decrease in editing efficiency (Li X et al., Nat Biotechnol, 2018, 36: 324-7).

Cytosine base editing is not wholly predictable; indels can occur at the target site, albeit at lower frequencies that those observed for C to T editing editors. Furthermore, cytosine base editors can occasionally cause C to A or C to G edits rather than the expected C to T edits. Adding linker lengths between Cas9 nickase and the rat APOBEC1 cytosine deaminase domain from 16 amino acids to 32 amino acids, the linker between the Cas9 nickase and the uracil glycosylase inhibitor from 4 amino acids to 9 amino acids, and using a second uracil glycosylase inhibitor was appended to the C-terminus of the new cytosine base editor using another 9 amino acid linker improved cytosine base editor termed “BE4” (Komor A C et al., Sci Adv, 2017, 3: eaao4774).

By fusing Escherichia coli adenine tTNA deaminase TadA (ecTadA) to dCas9 and mutagenesis of the ecTadA domain in conjunction with selection for editing activity revealed that A106V and D108N mutations yielded a base editor capable of editing adenine to guanine in DNA, termed ABE7.10 (Gaudelli N M et al., Nature, 2017, 551: 464-71). Koblan et al. improved the efficiency of ABE7.10 through modification of nuclear localization signals and codon optimization, yielding a version called ABEmax; a similar approach improved the efficiency of the cytosine base editor BE4.10 Huang et al. performed further development of both adenine and cytosine base editors to use alternative PAMs and to expand their editing windows, thereby increasing their targeting range (Koblan L W et al., Nat Biotechnol, 2018, 36: 843-6). The same has proven to be true of base editors, 12-14 although comparisons of Cas9, cytosine base editors, and adenine base editors using the same gRNAs have shown distinct off-target profiles.

The same has proven to be true of base editors, although comparisons of Cas9, cytosine base editors, and adenine base editors using the same gRNAs have shown distinct off-target profiles.

A variety of studies have raised concern about gRNA-independent off-target base editing, caused by the deaminase domain acting in isolation (without the need for engagement of DNA by the Cas9-gRNA complex). Additional studies showed that the gRNA-independent off-target effects of base editors are not limited to DNA. RNA sequencing of cells treated with either cytosine base editors or adenine base editors revealed transcriptome-wide off-target editing of RNA, and that introduction of amino acid substitution in the deaminase domain of adenine base (e.g. R106W) editors reduced off-target editing of RNA without substantially reducing on-target DNA base-editing efficiency. Zuo E, Sun Y, Wei W, et al. Cytosine base editor generates substantial off-target single-nucleotide variants in mouse embryos. Science, 2019, 364: 289-92; Jin S, Zong Y, Gao Q, et al. Cytosine, but not adenine, base editors induce genome-wide off-target mutations in rice. Science, 2019, 364: 292-5; Grünewald J, Zhou R, Garcia S P, et al. Transcriptome-wide off-target RNA editing induced by CRISPR-guided DNA base editors. Nature, 2019, 569: 433-7; Grünewald J, Zhou R, Iyer S, et al. CRISPR DNA base editors with reduced RNA off-target and self-editing activities. Nat Biotechnol, 2019, 37: 1041-8; Zhou C, Sun Y, Yan R, et al. Off-target RNA mutation induced by DNA base editing and its elimination by mutagenesis. Nature, 2019, 571: 275-8; Rees H A, Wilson C, Doman J L, et al. Analysis and minimization of cellular RNA editing by DNA adenine base editors. Sci Adv, 2019, 5: eaax5717, each of which is incorporated herein by reference in its entirety).

Correction of disease-causing mutations via precision editing with standard Cas9 genome editing has largely required HDR. Since HDR is limited to cells in S or G2 phase of mitosis, precision editing of non-mitotic cells is difficult. However, base editors are not reliant on HDR; the editing of postmitotic cochlear cells in mice is feasible with cytosine base editor BE3. By injecting BE3 and a gRNA in the form of a preassembled ribonucleoprotein via cationic liposomes, serine-33 in beta-catenin was edited to phenylalanine (TCT codon edited to TTT), allowing for the transdifferentiation of supporting cells into hair cells. Base editing of cochlear tissue was confirmed via sequencing, showing an editing rate between 0.7% and 3.0% depending on the region of the cochlea. In contrast, standard Cas9 editing via HDR showed negligible signs of efficacy in cochlear cells. A variant of SaBE3, delivered into the liver via adeno-associated viral (AAV) vectors, was reported to directly correct a pathogenic T to C mutation in the Pah gene with an editing rate as high as 29% and thereby treat the disease phenylketonuria in adult mice. Adenine base editing has also been demonstrated to generate mutations in mice. Yeh W H, Chiang H, Rees H A, et al. In vivo base editing of post-mitotic sensory cells. Nat Commun, 2018, 9: 2184; Villiger L, Grisch-Chan H M, Lindsay H, et al. Treatment of a metabolic liver disease by in vivo genome base editing in adult mice. Nat Med, 2018, 24: 1519-25; Ryu S M, Koo T, Kim K, et al. Adenine base editing in mouse embryos and an adult mouse model of Duchenne muscular dystrophy. Nat Biotechnol, 2018, 36: 536-9; Song C Q, Jiang T, Richter M, et al. Adenine base editing in an adult mouse model of tyrosinaemia. Nat Biomed Eng, 2020, 4: 125-30; Pisciotta L, Favari E, Magnolo L, et al. Characterization of three kindreds with familial combined hypolipidemia caused by loss-of-function mutations of ANGPTL3. Circ Cardiovasc Genet, 2012, 5: 42-50. each of which is incorporated herein by reference in its entirety.

Provided herein are compositions of nucleobase editor systems that comprises nucleobase editor proteins, complexes, or compounds that is capable of making a modification or conversion to a nucleobase (e.g., A, T, C, G, or U) within a target nucleotide sequence.

A nucleobase editor or a base editor (BE) refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating an adenine (A) in DNA.

In some embodiments, the base editor comprises a fusion protein comprising a programmable DNA binding protein fused to an adenosine deaminase. In some embodiments, the base editor comprises a fusion protein comprising a Cas9 protein and an adenosine deaminase. In some embodiments, the base editor is a Cas9 nickase (nCas9) fused to an adenosine deaminase. In some embodiments, the base editor is a nuclease-inactive Cas9 (dCas9) fused to an adenosine deaminase. In some embodiments, the base editor comprises a fusion protein comprising a programmable DNA binding protein fused to an cytidine deaminase. In some embodiments, the base editor comprises a fusion protein comprising a Cas9 protein and an cytidine deaminase. In some embodiments, the base editor is a Cas9 nickase (nCas9) fused to an cytidine deaminase. In some embodiments, the base editor is a nuclease-inactive Cas9 (dCas9) fused to an cytidine deaminase. In some embodiments, the base editor further comprises, an inhibitor of base excision repair, for example, a UGI domain. In some embodiments, the fusion protein comprises a Cas9 nickase fused to a deaminase and an inhibitor of base excision repair, such as a UGI or dISN domain. In some embodiments, the dCas9 domain of the fusion protein comprises a D10A and a H840A mutation as numbered in the wild type SpCas9 amino acid sequence. In some embodiments, the UGI comprises the following amino acid sequence:

>splP14739IUNGI_BPPB2 Uracil-DNA glycosylase inhibitor MTNLSDIIEKETGKQLVIQESILMLPEEVEEVIGNKPESDILVHTAYDESTDENVMLLT S D APE YKPW ALVIQDS NGENKIKML (SEQ ID NO: 735).

In some embodiments, a base editor system provided herein comprises a base editor fusion protein. For example, a base editor fusion protein may comprise a programmable DNA binding protein and a deaminase, e.g. an adenosine deaminase. In some embodiments, any of the fusion proteins provided herein are base editors. In some embodiments, the programmable DNA binding protein is a Cas9 domain, a Cpf1 domain, a CasX domain, a CasY domain, a Cas12b domain, a C2c2 domain, aC2c3 domain, or an Argonaute domain. In some embodiments, the programmable DNA binding protein is a Cas9 domain. The Cas9 domain may be any of the Cas9 domains or Cas9 proteins (e.g., nuclease inactive Cas9 or Cas9 nickase, or a Cas9 variant from any species) provided herein. In some embodiments, any of the Cas9 domains or Cas9 proteins provided herein may be fused with any of the deaminases provided herein. In some embodiments, the base editor comprises a deaminase, e.g., an adenosine deaminase and a programmable DNA binding protein, e.g., a Cas9 domain joined via a linker. In some embodiments, the base editor comprises a fusion protein comprising a deaminase, e.g., an adenosine deaminase and a programmable DNA binding protein, e.g., a Cas9 domain joined via a linker. In some embodiments, the linker is a peptide linker. In some embodiments, a linker is present between the deaminase domain and the Cas9 domain. In some embodiments, a deaminase and a programmable DNA binding domain are fused via any of the peptide linkers provided herein. For example, an adenosine deaminase and a Cas9 domain may be fused via a linker that comprises between 1 and 200 amino acids. In some embodiments, the adenosine deaminase and the programmable DNA binding protein are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, the adenosine deaminase and the programmable DNA binding protein are fused via a linker that comprises 4, 16, 32, or 104 amino acids in length. In some embodiments, the adenosine deaminase and the programmable DNA binding protein are fused via a linker that comprises the amino acid sequence of SGSETPGTSESATPES (SEQ ID NO: 736), SGGS (SEQ ID NO: 737), SGGSSGSETPGTSESATPESSGGS (SEQ ID NO: 738), SGGSSGGSSGSETPGTSESATPESSGGSSGGS (SEQ ID NO: 739), or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 740). In some embodiments, the adenosine deaminase and the programmable DNA binding protein are fused via a linker comprising the amino acid sequence SGSETPGTSESATPES (SEQ ID NO: 736), which may also be referred to as the XTEN linker. In some embodiments, the linker is 24 amino acids in length. In some embodiments, the linker comprises the amino acid sequence SGGSSGGSSGSETPGTSESATPES (SEQ ID NO: 741). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 742). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 743). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 744).

In some embodiments, a base editor system provided herein comprises a base editor comprising a fusion protein comprising an inhibitor of base repair. In some embodiments, a base editor comprises a fusion protein comprising a cytidine deaminase and a programmable DNA binding domain, e.g. a Cas9 domain. In some embodiments, a base editor comprises a fusion protein comprising an adenosine deaminase and a programmable DNA binding domain, e.g. a Cas9 domain. In some embodiments, the base editor or the fusion protein further comprises an inhibitor of base repair (IBR). In some embodiments, the IBR comprises an inhibitor of inosine base repair. In some embodiments, the IBR is an inhibitor of inosine base excision repair. In some embodiments, the inhibitor of inosine base excision repair is a catalytically inactive inosine specific nuclease (dISN). In some embodiments, a dISN may inhibit (e.g., by steric hindrance) inosine removing enzymes from excising the inosine residue from DNA. For example, catalytically dead inosine glycosylases (e.g., alkyl adenine glycosylase [AAG]) will bind inosine but will not create an abasic site or remove the inosine, thereby sterically blocking the newly-formed inosine moiety from potential DNA damage/repair mechanisms. Thus, this disclosure contemplates a fusion protein comprising a programmable DNA binding protein and an adenosine deaminase further fused to a dISN. This disclosure contemplates a fusion protein comprising any Cas9 domain, for example, a Cas9 nickase (nCas9) domain, a catalytically inactive Cas9 (dCas9) domain, a high fidelity Cas9 domain, or a Cas9 domain with reduced PAM exclusivity. It should be understood that the use of a dISN may increase the editing efficiency of a adenosine deaminase that is capable of catalyzing a A to I change. For example, fusion proteins comprising a dISN domain may be more efficient in deaminating A residues.

In some embodiments, the base editors provided herein comprise fusion proteins that further comprise one or more nuclear targeting sequences, for example, a nuclear localization sequence (NLS). In some embodiments, the fusion protein comprises multiple NLSs. In some embodiments, the fusion protein comprises a NLS at the N-terminus and the C-terminus of the fusion protein. In some embodiments, a NLS comprises an amino acid sequence that facilitates the importation of a protein, that comprises an NLS, into the cell nucleus. In some embodiments, the NLS is fused to the N-terminus of the fusion protein. In some embodiments, the NLS is fused to the C-terminus of the fusion protein. In some embodiments, the NLS is fused to the N-terminus of the programmable DNA binding protein, e.g. the Cas9. In some embodiments, the NLS is fused to the C-terminus of the programmable DNA binding protein. In some embodiments, the NLS is fused to the N-terminus of the adenosine deaminase. In some embodiments, the NLS is fused to the C-terminus of the adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, a NLS comprises the amino acid sequence PKKKRKV (SEQ ID NO: 745) or MDSLLMNRRKFLYQFKNVRWAKGRRETYLC (SEQ ID NO: 746). Additional nuclear localization sequences are known in the art and would be apparent to the skilled artisan. For example, NLS sequences are described in Plank et ah, PCT/EP2000/011690, the contents of which are incorporated herein by reference for their disclosure of exemplary nuclear localization sequences.

In some embodiments, the fusion proteins provided herein do not comprise a linker. In some embodiments, a linker is present between one or more of the domains or proteins (e.g., adenosine deaminase, napDNAbp, NLS, and/or IBR). In some embodiments, the “−” used in the general architecture above indicates the presence of an optional linker.

Some aspects of the disclosure provide base editors or fusion proteins that comprise a programmable DNA binding protein and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminase domains. In some embodiments, any of the fusion proteins provided herein comprise two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the first adenosine deaminase comprises any one of the mutations provided herein as numbered in SEQ ID NO: 747. In some embodiments, the second adenosine deaminase comprises any one of the mutations provided herein as numbered in SEQ ID NO: 747. In some embodiments, the first adenosine deaminase comprises any one of the mutations provided herein as numbered in SEQ ID NO: 747, and the second adenosine deaminase comprises a wild type adenosine deaminase sequence. In some embodiments, the second adenosine deaminase comprises any one of the mutations provided herein as numbered in SEQ ID NO: 747, and the first adenosine deaminase comprises a wild type adenosine deaminase sequence. As one example, the fusion protein may comprise a first adenosine deaminase and a second adenosine deaminase that both comprise a A106V, D108N, D147Y, and E155V mutation from ecTadA (SEQ ID NO: 747). As another example, the fusion protein may comprise a first adenosine deaminase domain that comprises a A106V, D108N, D147Y, and E155V mutation from ecTadA (SEQ ID NO: 747), and a second adenosine deaminase that comprises a L84F, A106V, D108N, H123Y, D147Y, E155V, and 1156F mutation from ecTadA (SEQ ID NO: 747).

In some embodiments, the adenosine deaminase comprises the amino acid sequence: MSEVEFSHEYWMRHALTLAKRAWDEREVPVGAVLVHNNRVIGEGWNRPIGRHDPTAH AEIMALRQGGLVMQNYRLIDATLYVTLEPCVMCAGAMIHSRIGRVVFGARDAKTGAAG SLMDVLHHPGMNHRVEITEGILADECAALLSDFFRMRRQEIKAQKKAQSSTD (SEQ ID NO: 747).

In some embodiments, the fusion protein comprises two adenosine deaminases (e.g., a first adenosine deaminase and a second adenosine deaminase). In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker. In some embodiments, the linker is any of the linkers provided herein, for example, any of the linkers described in the “Linkers” section. In some embodiments, the first adenosine deaminase is the same as the second adenosine deaminase. In some embodiments, the first adenosine deaminase and the second adenosine deaminase are any of the adenosine deaminases described herein. In some embodiments, the first adenosine deaminase and the second adenosine deaminase are different. In some embodiments, the first adenosine deaminase is any of the adenosine deaminases provided herein. In some embodiments, the second adenosine deaminase is any of the adenosine deaminases provided herein but is not identical to the first adenosine deaminase. In some embodiments, the first adenosine deaminase is an ecTadA adenosine deaminase. In some embodiments, the first adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid sequence set forth SEQ ID NO: 747 or to any of the adenosine deaminases provided herein. In some embodiments, the second adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid sequence set forth SEQ ID NO: 747 or to any of the adenosine deaminases provided herein. In some embodiments, the second adenosine deaminase comprises the amino acid sequence of SEQ ID NO: 747.

In some embodiments, the fusion proteins provided herein do not comprise a linker. In some embodiments, a linker is present between one or more of the domains or proteins (e.g., first adenosine deaminase, second adenosine deaminase, programmable DNA binding protein, and/or NLS).

It should be appreciated that the fusion proteins of the present disclosure may comprise one or more additional features. For example, in some embodiments, the fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, the fusion protein comprises an amino acid sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the amino acid sequences listed in Table 4. In some embodiments, the fusion protein comprises any one of the amino acid sequences listed in Table 4. In some embodiments, the sequence of the fusion protein is any one of the amino acid sequences listed in Table 4. In some embodiments, the fusion protein comprises an amino acid sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the amino acid sequences of SEQ ID NOs: 688, 691, 695, 702, 705, and 707. In some embodiments, the fusion protein comprises any one of the amino acid sequences of SEQ ID NOs: 688, 691, 695, 702, 705, and 707. In some embodiments, the sequence of the fusion protein is any one of the amino acid sequences of SEQ ID NOs: 688 and 702.

In some embodiments, the fusion protein is encoded by the polynucleotide sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences listed in Table 4. In some embodiments, the fusion protein is encoded by any one of the polynucleotide sequences listed in Table 23. In some embodiments, the fusion protein is expressed by the polynucleotide sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences listed in Table 4. In some embodiments, the fusion protein is expressed by any one of the polynucleotide sequences listed in Table 4. In some embodiments, the fusion protein is encoded by the polynucleotide sequence that comprises at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences of SEQ ID NOs: 687, 690, 694, 701, 704, and 706. In some embodiments, the fusion protein is encoded by the polynucleotide sequence that comprises any one of the polynucleotide sequences of SEQ ID NOs: 687, 690, 694, 701, 704, and 706. In some embodiments, the fusion protein is encoded by any one of the polynucleotide sequences of SEQ ID NOs: 687 and 701. In some embodiments, the fusion protein is expressed by the polynucleotide sequence that comprises at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences of SEQ ID NOs: 687, 690, 694, 701, 704, and 706, or a combination thereof. In some embodiments, the fusion protein is encoded by the polynucleotide sequence that comprises any one of the polynucleotide sequences of SEQ ID NOs: 687, 690, 694, 701, 704, and 706, or a combination thereof. In some embodiments, the fusion protein is expressed by any one of the polynucleotide sequences of SEQ ID NOs: 687, 690, 694, 701, 704, and 706. In some embodiments, the polynucleotide sequence further comprises at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences of SEQ ID NOs: 687 and 701.

In some embodiments, the nucleobase editor ABE8.8 comprises a fusion protein comprising the sequence as provided below:

(SEQ ID NO: 734)
MSEVEFSHEYWMRHALTLAKRARDEREVPVGAVLVLNNRVIGEGWNRAIGLHDPTAH
AEIMALRQGGLVMQNYRLIDATLYVTFEPCVMCAGAMIHSRIGRVVFGVRNAKTGAAG
SLMDVLHHPGMNHRVEITEGILADECAALLCRFFRMPRRVFNAQKKAQSSTDSGGSSGG
SSGSETPGTSESATPESSGGSSGGSDKKYSIGLAIGTNSVGWAVITDEYKVPSKKFKVLGN
TDRHSIKKNLIGALLFDSGETAEATRLKRTARRRYTRRKNRICYLQEIFSNEMAKVDDSF
FHRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNRED
LLRKQRTFDNGSIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPKHSLLYEYF
TVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVV
KKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSIT
GLYETRIDLSQLGGD.

Amino acid and nucleotide changes of MA040, MA041, and MA045 compared to MA004: MA004 has codon CGG at nCas9 amino acid position 691 and codon GAC at amino acid position 1135. MA040 has a D1135E nCas9 amino acid mutation; codon 1135 is changed to GAG. MA041 has R691A and D1135E nCas9 amino acid mutations; codon 691 is changed to GCC and co-don 1135 is changed to GAG. MA045 has a R691A nCas9 amino acid mutation; codon 691 is changed to GCC.

Additional adenosine deaminase mutations and variants are described in Patent Application WO2018119354, which is incorporated herein by reference in its entirety.

Additional adenosine deaminases useful in the present application would be apparent to the skilled artisan and are within the scope of this disclosure. For example, the adenosine deaminase may be a homolog of an AD AT. Exemplary AD AT homologs include, without limitation:

Staphylococcus aureus TadA:

(SEQ ID NO: 748)
MGSHMTNDIYFMTLAIEEAKKAAQLGEVPIGAIITKDDEVIARAHNLRE
TLQQPTAHAEHIAIERAAKVLGSWRLEGCTLYVTLEPCVMCAGTIVMSR
IPRVVYGADDPKGGCSGSLMNLLQQSNFNHRAIVDKGVLKEACSTLLTT
FFKNLRANKKSTN
Bacillus subtilis TadA:
(SEQ ID NO: 749)
MTQDELYMKEAIKEAKKAEEKGEVPIGAVLVINGEIIARAHNLRETEQR
SIAHAEMLVIDEACKALGTWRLEGATLYVTLEPCPMCAGAVVLSRVEKV
VFGAFDPKGGCSGTLMNLLQEERFNHQAEVVSGVLEEECGGMLSAFFRE
LRKKKKAARKNLSE
Salmonella typhimurium (S. typhimurium) TadA:
(SEQ ID NO: 750)
MPPAFITGVTSLSDVELDHEYWMRHALTLAKRAWDEREVPVGAVLVHNH
RVIGEGWNRPIGRHDPTAHAEIMALRQGGLVLQNYRLLDTTLYVTLEPC
VMCAGAMVHSRIGRVVFGARDAKTGAAGSLIDVLHHPGMNHRVEIIEGV
LRDECATLLSDFFRMRRQEIKALKKADRAEGAGPAV
Shewanella putrefaciens (S. putrefaciens) TadA:
(SEQ ID NO: 751)
MDEYWMQVAMQMAEKAEAAGEVPVGAVLVKDGQQIATGYNLSISQHDPT
AHAEILCLRSAGKKLENYRLLDATLYITLEPCAMCAGAMVHSRIARVVY
GARDEKTGAAGTVVNLLQHPAFNHQVEVTSGVLAEACSAQLSRFFKRRR
DEKKALKLAQRAQQGIE
Haemophilus influenzae F3031 (H. influenzae) TadA:
(SEQ ID NO: 752)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAI
LHSRIKRLVFGASDYKTGAIGSRFHFFDDYKMNHTLEITSGVLAEECSQ
KLSTFFQKRREEKKIEKALLKSLSDK
Caulobacter crescentus (C. crescentus) TadA:
(SEQ ID NO: 753)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAI
SHARIGRVVFGADDPKGGAVVHGPKFFAQPTCHWRPEVTGGVLADESAD
LLRGFFRARRKAKI
Geobacter sulfurreducens (G. sulfurreducens) TadA:
(SEQ ID NO: 754)
MSSLKKTPIRDDAYWMGKAIREAAKAAARDEVPIGAVIVRDGAVIGRGH
NLREGSNDPSAHAEMIAIRQAARRSANWRLTGATLYVTLEPCLMCMGAI
ILARLERVVFGCYDPKGGAAGSLYDLSADPRLNHQVRLSPGVCQEECGT
MLSDFFRDLRRRKKAKATPALFIDERKVPPEP

In other aspects, the disclosure provides base-editing systems and methods for editing a polynucleotide. For example, provided herein are genome/base-editing systems and methods for editing a polynucleotide encoding ANGPTL3 and variants thereof. To edit a target gene, a target gene polynucleotide may contact the systems disclosed herein comprising a sgRDNA and a adenosine base editor protein, wherein the sgRDNA comprises a spacer sequence as disclosed herein and a tracrRNA sequence, wherein the spacer sequence hybridizes with a target polynucleotide sequence in a ANGPTL3 gene and the tracrRNA sequence binds the adenosine base editor protein (e.g. a Cas9 component of the adenosine base editor). The sgRDNA therefore can direct the base editor protein to the target polynucleotide sequence to result in a to G modification in the target gene. The target gene or target polynucleotide can be a gene encoding ANGPTL3. The target polynucleotide sequence can be in an ANGPTL3 gene. The modification can reduce or abolish expression of functional ANGPTL3 protein encoded by the ANGPTL3 gene in the cell. The introduction can be performed via a lipid nanoparticle that comprises the composition.

For example, the sgRDNA and the Adenosine base editor protein may be expressed in a cell where a target gene editing is desired (e.g., a liver cell), to thereby allowing contact of the target gene with the composition disclosed herein (e.g., sgRNA and the Adenosine base editor protein). In some embodiments, the binding of the Adenosine base editor protein to its target polynucleotide sequence in the target gene is directed by a single guide RDNA disclosed herein, e.g., a single guide RDNA comprising (i) a spacer sequence as disclosed herein and (ii) a tracrRNA sequence, wherein the spacer sequence hybridizes with a target polynucleotide sequence in a target gene. Thus, by designing the guide RDNA sequence, the Adenosine base editor protein can be directed to edit any target polynucleotide sequence in the target gene (e.g., target gene encoding ANGPTL3). The guide RDNA sequence can be co-expressed with the Adenosine base editor protein in a cell where editing is desired. To edit a gene encoding the ANGPTL3 protein, the gene is contacted with the systems described herein. A target polynucleotide sequence in a target gene can be contacted with the single guide RDNA disclosed herein and an adenosine base editor protein or a nucleic acid sequence encoding the Adenosine base editor protein, wherein the single guide RDNA directs the Adenosine base editor protein to effect a modification in the target gene (e.g., target gene encoding ANGPTL3). The target polynucleotide sequence can be the gene locus in the genomic DNA of a cell. The cell can be a cultured cell. The cell may be in vivo, in vitro, or ex vivo.

A base editor system provided herein may comprise a programmable DNA binding protein and a deaminase. As used herein, a deaminase may refer to an enzyme that catalyzes the removal of an amine group from a molecule, or deamination, for example through hydrolysis. In some embodiments, the deaminase is a cytidine deaminase, catalyzing the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T, as described in Komor et al, Nature, Programmable editing of a target base in genomic DNA without double-stranded DNA cleavage, 533, 420-424 (2016), which is incorporated herein by reference in its entirety. In some embodiments, the deaminase is a cytosine deaminase, catalyzing and promoting the conversion of cytosine to uracil (e.g., in RNA) or thymine (e.g., in DNA). In some embodiments, the deaminase is an adenosine deaminase, catalyzing and promoting the conversion of adenine to guanine. In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variants do not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A cytidine deaminase (or cytosine deaminase) comprises an enzyme that catalyzes the chemical reaction “cytosine+H20->uracil+NH3” or “5-methyl-cytosine+H20->thymine+NH3.” In the context of a gene, such nucleotide change, or mutation, may in turn lead to an amino acid change in the protein, which may affect the protein's function, e.g., loss-of-function or gain-of-function. Subsequent DNA repair mechanisms ensure that uracil bases in DNA are replaced by T, as described in Komor et al. (Nature, Programmable editing of a target base in genomic DNA without double-stranded DNA cleavage, 533, 420-424 (2016), which is incorporated herein by reference in its entirety).

One exemplary suitable class of cytosine deaminases is the apolipoprotein B mRNA-editing complex (APOBEC) family of cytosine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytosine deaminases all require a Zn-coordinating motif (His-X-Glu-X23_26-Pro-Cys-X2_4-Cys (SEQ ID NO: 29)) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded (3-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytosine deaminase is the activation-induced cytidine deaminase (AID), which is responsible for the maturation of antibodies by converting cytosines in ssDNA to uracils in a transcription-dependent, strand-biased fashion.

An adenosine deaminase (or adenine deaminase) comprises an enzyme that catalyzes the hydrolytic deamination of adenosine or deoxy adenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase. In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid sequence set forth in SEQ ID NO:747 or to any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to the amino acid sequence set forth in SEQ ID NO: 747 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NO: 747 or any of the adenosine deaminases provided herein. Additional exemplary adenosine deaminase can be found in PCT Application Publication NO. WO2021/207712, which is hereby incorporated by reference in its entirety.

In some embodiments, the adenine base editor comprises an amino acid sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the amino acid sequences listed in Table 2 or Table 4. In some embodiments, the adenine base editor comprises any one of the amino acid sequences listed in Table 2 or Table 4. In some embodiments, the sequence of the adenine base editor is any one of the amino acid sequences listed in Table 2 or Table 4. In some embodiments, the adenine base editor comprises an amino acid sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the amino acid sequences of SEQ ID NOs: SEQ ID NOs: 688, 691, 695, 702, 705, 707, 718, 720, 722, 724, 726, 728 and 730. In some embodiments, the adenine base editor comprises any one of the amino acid sequences of SEQ ID NOs: 688, 691, 695, 702, 705, 707, 718, 720, 722, 724, 726, 728 and 730. In some embodiments, the sequence of the adenine base editor is any one of the amino acid sequences of SEQ ID NOs: 688, 702, 718, 720, 722, 724, 726, 728 and 730.

In some embodiments, the adenine base editor is encoded by the polynucleotide sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences listed in Table 2 or Table 4. In some embodiments, the adenine base editor is encoded by any one of the polynucleotide sequences listed in Table 2 or Table 4. In some embodiments, the adenine base editor is expressed by the polynucleotide sequence that is at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences listed in Table 2 or Table 4. In some embodiments, the adenine base editor is expressed by any one of the polynucleotide sequences listed in Table 2 or Table 4. In some embodiments, the adenine base editor is encoded by the polynucleotide sequence that comprises at least 60%, at least 65%, at least 70%, at least 75%, at least 80%, at least 85%, at least 90%, at least 95%, at least 96%, at least 97%, at least 98%, at least 99%, at least 99.5%, at least 99.7%, or at least 99.9% identical to any one of the polynucleotide sequences of SEQ ID NOs: 687, 690, 694, 701, 704, 706, 717, 719, 721, 723, 725, 727, and 729. In some embodiments, the adenine base editor is encoded by the polynucleotide sequence that comprises any one of the polynucleotide sequences of SEQ ID NOs: 687, 690, 694, 701, 704, 706, 717, 719, 721, 723, 725, 727, and 729. In some embodiments, the adenine base editor is encoded by any one of the polynucleotide sequences of SEQ ID NOs: 687, 701, 717, 719, 721, 723, 725, 727, and 729.

### Prime Editing

The disclosed gene editing systems can encompass a prime editing CRISPR system or a component thereof. Prime editing is a variation on CRISPR systems which expands the guide RNA's responsibility to serve two purposes: to guide Cas9 to a targeted genomic location, and to serve as an RNA template to copy new sequences into the DNA genome (Anzalone A V et al., Nature volume 576, pages 149-157 (2019)). Similar to CRISPR, prime editing requires the presence of a catalytically modified Cas endonuclease and a single guide RNA. The Cas9 endonuclease is catalytically modified to be a Cas9 nickase which nicks the DNA rather than generating a double-strand break. The Cas9 nickase is fused to a reverse transcriptase. The prime editing guide RNA (pegRNA), is substantially larger than standard sgRNA. The pegRNA is a sgRNA with a primer binding sequence (PBS) and the template containing the desired RNA sequence added at the 3′end. Additional information about prime editing can be found in published PCT application WO2020191245A1, which is incorporated herein in its entirety.

The hybrid guide gene editing systems disclosed herein can comprise a polymerase. A polymerase functions to catalyze the polymerization of a nucleic acid strand using an existing nucleic acid as a template. Examples of useful polymerases include DNA polymerases and RNA polymerases. The polymerase can work with a nucleic acid programmable nucleotide binding domain or a nucleic acid programmable DNA binding protein (e.g., in the form of fusion proteins or coupled or associated in trans with the hybrid guide nucleic acid sequences). The polymerase can be a RNA-dependent DNA polymerase (e.g., reverse transcriptase) or a DNA-dependent DNA polymerase (e.g., a prokaryotic polymerase, including Pol I, Pol II, or Pol III, or a eukaryotic polymerase, including Pol a, Pol b, Pol g, Pol d, Pol e, or Pol z).

### Editing Efficiency

The gene editing systems disclosed herein can achieve high efficiency in gene editing with low off-target editing effect. For example, the gene editor protein in the gene editing system with guide nucleic acids comprising deoxyribonucleotide containing motif(s) in the spacer disclosed herein can affect less than 10% editing on all off-target sites as compared to a gene editing system with guide nucleic acid sequence without deoxyribonucleotide. For example, the gene editor protein in the gene editing system with the guide nucleic acid sequence comprising any of the motifs in the spacer sequence disclosed herein can affect less than 7% editing on all off-target sites as compared to a gene editing system with guide nucleic acid sequence without deoxyribonucleotide. The gene editor protein in the gene editing system with the guide nucleic acid sequence comprising any of the motifs in the spacer sequence disclosed herein can affect less than 5% editing on all off-target sites as compared to a gene editing system with guide nucleic acid sequence without deoxyribonucleotide. The gene editor protein in the gene editing system with the guide nucleic acid sequence comprising any of the motifs in the spacer sequence disclosed herein can affect less than 2% editing on all off-target sites as compared to a gene editing system with guide nucleic acid sequence without deoxyribonucleotide. The gene editor protein in the gene editing system with the guide nucleic acid sequence comprising any of the motifs in the spacer sequence disclosed herein can affect less than 1% editing on all off-target sites as compared to a gene editing system with guide nucleic acid sequence without deoxyribonucleotide. The gene editor protein in the gene editing system with the guide nucleic acid sequence comprising any of the motifs in the spacer sequence disclosed herein can affect less than 10%, 9%, 8%, 7%, 6%, 5%, 4%, 3%, 2%, or 1% editing on all off-target sites as compared to a gene editing system with guide nucleic acid sequence without deoxyribonucleotide.

The gene editor protein in the gene editing system with the guide nucleic acid sequence comprising any of the motifs in the spacer sequence disclosed herein can affect at least 50% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect at least 70% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect at least 90% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect more than 50% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect more than 70% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect more than 90% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect at least 50%, 60%, 70%, 80%, or 90% editing on the target gene as compared to a gene editing system with guide nucleic acid sequence without deoxyribonucleotide. The gene editor protein in the gene editing system with the guide nucleic acid sequence comprising any of the motifs in the spacer sequence disclosed herein can affect more than 50%, 60%, 70%, 80%, or 90% editing on the target gene as compared to a gene editing system with guide nucleic acid sequence without deoxyribonucleotide.

### Gene Editor Protein

Provided herein are gene editor proteins comprising a nucleic acid-binding domain. The nucleic acid-binding domain may be able to bind to DNA. The nucleic acid-binding domain may be able to bind to RNA. The nucleic acid-binding domain can be a Cas9 domain. The term “Cas9” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to as a Casn1 nuclease or a CRISPR (clustered regularly interspaced short palindromic repeat) associated nuclease. Cas9 can refer to a polypeptide with at least or at least about 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most or at most about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

Cas9 nuclease sequences and structures of variant Cas9 orthologs have been described in various species. Exemplary species that the Cas9 protein or other components can be from include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polar omonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillator ia sp., Petrotoga mobilis, Thermosipho africanus, Streptococcus pasteurianus, Neisseria cinerea, Campylobacter lari, Parvibaculum lavamentivorans, Coryne bacterium diphtheria, or Acaryochloris marina. In some embodiments, the Cas9 protein is from Streptococcus pyogenes. In some embodiments, the Cas9 protein may be from Streptococcus thermophilus. In some embodiments, the Cas9 protein is from Staphylococcus aureus.

Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference.

The gene editing systems provided herein can comprise a gene editor protein, e.g. a Cas nuclease, with reduced or abolished nuclease activity. For example, a Ca9 protein may be nuclease inactive or may be a Cas9 nickase. Methods for generating a Cas9 protein (or a fragment thereof) having an inactive DNA cleavage domain are known (See, e.g., Jinek et al., Science. 337:816-821 (2012); Qi et al, Cell. 28; 152(5): 1173-83 (2013)). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al, Cell. 28;152(5): 1173-83 (2013)). The Cas9 nickase suitable for use in accordance with the present disclosure has an active HNH domain and an inactive RuvC domain and is able to cleave only the strand of the target DNA that is bound by the sgRNA (which is the opposite strand of the strand that is being edited via cytidine deamination). The Cas9 nickase of the present disclosure may comprise mutations that inactivate the RuvC domain, e.g., a D10A mutation. It is to be understood that any mutation that inactivates the RuvC domain may be included in a Cas9 nickase, e.g., insertion, deletion, or single or multiple amino acid substitution in the RuvC domain. In a Cas9 nickase described herein, while the RuvC domain is inactivated, the HNH domain remains activate. Thus, while the Cas9 nickase may comprise mutations other than those that inactivate the RuvC domain (e.g., D10A), those mutations do not affect the activity of the HNH domain. In a non-limiting Cas9 nickase example, the histidine at position 840 remains unchanged.

Additional suitable mutations that inactivate Cas9 will be apparent to those of skill in the art based on this disclosure and knowledge in the field, and are within the scope of this disclosure. Such additional exemplary suitable nuclease-inactive Cas9 domains include, but are not limited to, D839A and/or N863A (See, e.g., Prashant et al, Nature Biotechnology. 2013; 31(9): 833-838, which are incorporated herein by reference), or K603R (See, e.g., Chavez et al., Nature Methods 12, 326-328, 2015, which is incorporated herein by reference). Cas9, dCas9, or Cas9 variant also encompasses Cas9, dCas9, or Cas9 variants from any organism. Also appreciated is that dCas9, Cas9 nickase, or other appropriate Cas9 variants from any organisms may be used in accordance with the present disclosure.

Cas9 can be a Cas9 from archaea (e.g., nanoarchaea), which constitute a domain and kingdom of single-celled prokaryotic microbes.

The gene editor protein can comprise a CasX or CasY, or a variant thereof, which have been described in, for example, Burstein et al., Cell Res. 2017 Feb. 21. doi: 10.1038/cr.2017.21.

The gene editor proteins can comprise high fidelity Cas9 domains. High fidelity Cas9 domains are engineered Cas9 domains comprising one or more mutations that decrease electrostatic interactions between the Cas9 domain and the sugar-phosphate backbone of DNA, as compared to a corresponding wild-type Cas9 domain. Without wishing to be bound by any particular theory, high fidelity Cas9 domains that have decreased electrostatic interactions with the sugar-phosphate backbone of DNA may have less off-target effects. The Cas9 domain can comprise one or more mutations that decreases the association between the Cas9 domain and the sugar-phosphate backbone of DNA. A Cas9 domain can comprise one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, or more. Any of the Cas9 provided herein can comprise one or more of N497X, R661X, Q695X, and/or Q926X mutation as numbered in the wild type Cas9 amino acid sequence or a corresponding amino acid in another Cas9, wherein X is any amino acid. The Cas9 can comprise one or more of N497A, R661A, Q695A, and/or Q926A mutation of the amino acid sequence provided in the wild type Cas9 sequence, or a corresponding mutation as numbered in the wild type Cas9 amino acid sequence or a corresponding amino acid in another Cas9. Cas9 domains with high fidelity have been described in Kleinstiver, B. P., et al., Nature 529, 490-495 (2016); and Slaymaker, I. M., et al., Science 351, 84-88 (2015); the entire contents of each are incorporated herein by reference.

It should be appreciated that any of the gene editing systems provided herein, may be converted into high fidelity gene editing systems by modifying the Cas9 domain as described herein to generate high fidelity gene editor protein comprising the high fidelity Cas9 domain. The high fidelity Cas9 domain can be a nuclease inactive Cas9 domain or a Cas9 nickase domain.

The gene editor protein can comprise one or more nuclease domains. A gene editor protein of the disclosure can comprise a HNH or HNH-like nuclease domain, a RuvC or RuvC-like nuclease domain, and/or HEPN-superfamily-like nucleases. HNH or HNH-like domains can comprise a McrA-like fold. HNH or HNH-like domains can comprise two antiparallel O-strands and an α-helix. HNH or HNH-like domains can comprise a metal binding site (e.g., divalent cation binding site). HNH or HNH-like domains can cleave one strand of a target nucleic acid (e.g., complementary strand of the crRNA targeted strand). Proteins that comprise an HNH or HNH-like domain can include endonucleases, colicins, restriction endonucleases, transposases, and DNA packaging factors.

A gene editor protein can be a Cas9 protein, a Cpf1 protein, a C2c1 protein, a C2c2 protein, a C2c3 protein, Cas3, Cas 5, Cas7, Cas8, Cas10, or complexes of these, dependent upon the particular CRISPR system being used. The gene editor protein can be a Cas9 or a Cpf1 protein. A gene editor protein can have reduced nuclease activity. In some instances, the gene editor protein is a nickase, i.e., it can be modified to cleave one strand of a target nucleic acid duplex. A gene editor protein can be modified to have no nuclease activity, i.e., it does not cleave any strand of a target nucleic acid duplex, or any single strand of a target nucleic acid. Examples of gene editor protein with reduced or no nuclease activity include, but not limited to, a Cas9 with a modification to the HNH and/or RuvC nuclease domains, and a Cpf1 with a modification to the RuvC nuclease domain. Non-limiting examples of such modifications can include D917A, ET006A and D1225A to the RuvC nuclease domain of the F. novicida Cpf1 and alteration of residues D10, G12, G17, E762, H840, N854, N863, H982, H983, A984, D986, and/or A987 of the S. pyogenes Cas9, and their corresponding amino acid residues in other Cpf1 and Cas9 proteins.

Any variants of Cas9 known in the art can also be the gene editor protein. In some instances, the variant of Cas9 is a catalytically inactive Cas9 (dCas9). A dCas9 can bear mutations at two nuclease domains and thus lack nuclease activity. The dCas9 can function as a programmable sequence-specific DNA-binding protein. dCas9 can be used to physically block the process of transcription, turning off a specific gene, or to shuttle other proteins to a particular site in the genome. As used herein, a “dCas” and “dCas protein” are used interchangeably and refer to a catalytically inactive CRISPR associated protein. In some instances, the dCas protein comprises one or more mutations in a DNA-cleavage domain. The dCas protein can comprise one or more mutations in the RuvC or HNH domain. The dCas protein can comprise one or more mutations in both the RuvC and HNH domain. The dCas can be a fragment of a wild-type Cas molecule. The dCas can comprise a functional domain from a wild-type Cas molecule, wherein the functional domain is chosen from a Reel domain, a bridge helix domain, or a PAM interacting domain. The nuclease activity of the dCas molecule can be reduced by at least 40%, 45%, 50%, 55%, 60%, 65%, 70%, 75%, 80%, 85%), 90%), 95%), 99% compared to that of a corresponding wild type Cas.

Suitable dCas can be derived from a wild type Cas. The Cas can be from a type I, type II, or type III CRISPR-Cas systems. In some instances, suitable dCas proteins are derived from a Cas1, Cas2, Cas3, Cas4, Cas5, Cas6, Cas7, Cas8, Cas9, or Cas10 molecule. The dCas can be derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 sgRNA and DNA binding activity. In some instances, the two point mutations within the RuvC and HNH active sites are DIOA and H840A mutations of the S. pyogenes Cas9 molecule. Alternatively, D10 and H840 of the S. pyogenes Cas9 molecule can be deleted to abolish the Cas9 nuclease activity while retaining its sgRNA and DNA binding activity. In some instances, the two point mutations within the RuvC and HNH active sites are DIOA and N580A mutations of the S. aureus Cas9 molecule. The dCas can be an S. aureus dCas9 molecule comprising a mutation at D10 and/or N580. The dCas can be an S. aureus dCas9 molecule comprising DIOA and/or N580A mutations. The dCas can be an S. aureus dCas9 molecule, any variant or mutant, or any fragment thereof.

Similar mutations can also apply to any other naturally-occurring Cas9 (e.g., Cas9 from other species) or engineered Cas9 molecules. The dCas9 can comprise a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof.

The gene editor protein disclosed herein can be modified. Such modifications may include the incorporation or fusion of a domain from another polypeptide to the gene editor protein, or replacement of a domain of the gene editor protein with a domain of another polypeptide. For example, a modified the gene editor protein can contain a first domain from a Cas9 or Cpf1 protein and a second domain from a protein other than Cas9 or Cpf1. The modification to include such domains in the modified gene editor protein may confer additional activity on the modified gene editor protein. Such activities can include nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity) that modifies a polypeptide associated with target nucleic acid (e.g., a histone).

The gene editor protein can introduce double-stranded breaks or single-stranded breaks in nucleic acid sequences, (e.g., genomic DNA). A nucleic acid sequence can be a target nucleic acid. The gene editor protein can introduce blunt-end cleavage sites or produce cleavage sites having sticky ends, i.e., 5′ or 3′ overhangs. Cpf1, for example, may introduce a staggered DNA double-stranded break with about a 4 or 5 nucleotide (nt) 5′ overhang. A double-stranded break can stimulate a cell's endogenous DNA-repair pathways (e.g., NHEJ or alternative non-homologous end-joining (A-NHEJ). NHEJ can repair a cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by the gene editor protein, the site of cleavage can be destroyed (e.g., the site may not be accessible for another round of cleavage with a nucleic acid-targeting polynucleotide and site-directed polypeptide).

The gene editor protein can comprise a nucleic acid binding domain and thus can bind a nucleic acid. The nucleic acid can be a DNA sequence. The DNA sequence can be a target DNA sequence. The nucleic acid binding domain can be a DNA binding domain.

Any DNA-binding domain can be used in the systems disclosed herein. The DNA binding domain can comprise a zinc finger protein. The zinc finger protein can be non-naturally occurring in that it is engineered to bind to a target site of choice. Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

The systems described herein can employ a meganuclease (homing endonuclease) DNA-binding domain for binding to the target nucleic acid. Naturally-occurring meganucleases recognize 15-40 base-pair cleavage sites and are commonly grouped into four families: the LAGLIDADG family, the GIY-YIG family, the His-Cyst box family, and the HNH family. Exemplary homing endonucleases include I-SceI, I-Ceul, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

The DNA-binding domain of one or more of the nucleases used in the methods and compositions described herein comprises a naturally occurring or engineered (non-naturally occurring) TAL effector DNA binding domain. See, e.g., U.S. Pat. No. 8,586,526, incorporated by reference in its entirety herein.

### Deaminase Domains

The gene editing systems disclosed herein can comprise a deaminase. As used herein, a “deaminase” may refer to an enzyme that catalyzes the removal of an amine group from a molecule, or deamination, for example through hydrolysis. The deaminase can be a cytidine deaminase, catalyzing the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T, as described in Komor et al, Nature, Programmable editing of a target base in genomic DNA without double-stranded DNA cleavage, 533, 420-424 (2016), which is incorporated herein by reference in its entirety. The deaminase can be a cytosine deaminase, catalyzing and promoting the conversion of cytosine to uracil (e.g., in RNA) or thymine (e.g., in DNA). The deaminase can be an adenosine deaminase, catalyzing and promoting the conversion of adenine to guanine. The deaminase can be a naturally-occurring deaminase from an organism, such as a human, chimpanzee, gorilla, monkey, cow, dog, rat, or mouse. The deaminase can be a variant of a naturally-occurring deaminase from an organism, and the variants do not occur in nature. For example, the deaminase or deaminase domain can be at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A cytidine deaminase (or cytosine deaminase) comprises an enzyme that catalyzes the chemical reaction “cytosine+H20→uracil+NH3” or “5-methyl-cytosine+H20→thymine+NH3.” In the context of a gene, such nucleotide change, or mutation, may in turn lead to an amino acid change in the protein, which may affect the protein's function, e.g., loss-of-function or gain-of-function. Subsequent DNA repair mechanisms ensure that uracil bases in DNA are replaced by T, as described in Komor et al. (Nature, 533, 420-424 (2016), which is incorporated herein by reference in its entirety).

One exemplary suitable class of cytosine deaminases is the apolipoprotein B mRNA-editing complex (APOBEC) family of cytosine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytosine deaminases all require a Zn-coordinating motif (His-X-Glu-X23_26-Pro-Cys-X2_4-Cys) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded R-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytosine deaminase is the activation-induced cytidine deaminase (AID), which is responsible for the maturation of antibodies by converting cytosines in ssDNA to uracils in a transcription-dependent, strand-biased fashion.

An adenosine deaminase (or adenine deaminase) comprises an enzyme that catalyzes the hydrolytic deamination of adenosine or deoxy adenosine to inosine or deoxyinosine, respectively. The adenosine deaminase can catalyze the hydrolytic deamination of adenine or adenosine in deoxyribonucleic acid (DNA). The adenosine deaminases (e.g., engineered adenosine deaminases, evolved adenosine deaminases) provided herein can be from any organism, such as a bacterium. The deaminase or deaminase domain can be a variant of a naturally-occurring deaminase from an organism. The deaminase or deaminase domain may not occur in nature. For example, the deaminase or deaminase domain can be at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. The adenosine deaminase can be from a bacterium, such as, E. coli, S. aureus, S. typhi, S. putrefaciens, H. influenzae, or C. crescentus. The adenosine deaminase can be a TadA deaminase. The TadA deaminase can be an E. coli TadA deaminase. The TadA deaminase can be a truncated E. coli TadA deaminase. For example, the truncated ecTadA may be missing one or more N-terminal amino acids relative to a full-length ecTadA. The truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. The truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. The ecTadA deaminase may not comprise an N-terminal methionine.

It should be appreciated that adenosine deaminases provided herein may include one or more mutations. Exemplary adenosine deaminase mutations and variants are described in Patent Application WO2018119354, which is incorporated herein by reference in its entirety. Additional adenosine deaminases useful in the present application would be apparent to the skilled artisan and are within the scope of this disclosure.

### Guide Nucleic Acid

A guide nucleotide sequence can exist as a single nucleotide molecule and comprise two domains: (1) a domain that shares homology to a target nucleic acid and directs binding of a guide nucleotide sequence-gene editor protein to the targeted gene sequence; and (2) a domain that binds a guide nucleotide sequence-gene editor protein. Domain (1) can comprise a spacer sequence. Domain (2) can be referred to as a tracrRNA sequence or equivalent such as a modified tracrRNA. Domain (2) may comprise a stem-loop structure. For example, domain (2) can be identical or homologous to a tracrRNA as provided in Jinek et al., Science 337:816-821(2012), which is incorporated herein by reference. Other examples of gRNAs (e.g., those including domain (2)) can be found in U.S. Patent Application Publication US20160208288 and U.S. Patent Application Publication US20160200779 each of which is herein incorporated by reference in their entirety.

Methods of using guide nucleotide sequence-gene editor protein, such as Cas9, for site-specific cleavage (e.g., to modify a genome) are known in the art (see e.g., Cong, L. et al., Science 339, 819-823 (2013); Mali, P. et al., Science 339, 823-826 (2013); Hwang, W. Y. et al., Nature biotechnology 31, 227-229 (2013); Jinek, M. et al., eLife 2, e00471 (2013); Dicarlo, J. E. et al., Nucleic acids research (2013); Jiang, W. et al., Nature biotechnology 31, 233-239 (2013); each of which are incorporated herein by reference).

The guide nucleic acids of the gene editing systems disclosed herein can comprise a DNA-RNA chimera guide (i.e., chRDNA). The chRDNA can be a single guide RDNA. As used herein, the term “RDNA” refers to a nucleic acid comprising a mixture of ribonucleotide (RNA) and deoxynucleotide (DNA). That is, an RDNA comprises at least one ribonucleotide and at least one deoxynucleotide.

As used herein, the term “sgchRDNA,” “sgRDNA,” “single guide chRDNA,” and “single guide RDNA” are used interchangeably and refer to a polynucleotide comprising a spacer sequence, wherein the spacer sequence comprises a mixture of DNA and RNA nucleotides that is complementary to a sequence in a target nucleic acid. The spacer sequence comprises at least one deoxyribonucleotide and at least one ribonucleotide. The ribose of the ribonucleotide of the spacer sequence can be modified. For example, the ribose can comprise a 2′ hydroxyl group covalently linked to a methyl group (2′-O-Methyl).

As used herein, the term “sgRNA,” “sgRNA,” “single guide RNA,” and “single guide RNA” are used interchangeably and refer to a polynucleotide comprising a spacer sequence, wherein the spacer sequence comprises solely RNA that is complementary to a sequence in a target nucleic acid. The spacer sequence comprises only ribonucleotide. The ribose of the ribonucleotide of the spacer sequence can be modified. For example, the ribose can comprise a 2′ hydroxyl group covalently linked to a methyl group (2′-O-Methyl).

The guide nucleic acid disclosed herein can comprise, for example, a deoxyribonucleotide-deoxyribonucleotide-ribonucleotide-deoxyribonucleotide-deoxyribonucleotide (dN-dN-N-dN-dN) motif. The guide nucleic acid disclosed herein can comprise, in another example, a deoxyribonucleotide-deoxyribonucleotide-ribonucleotide-deoxyribonucleotide-deoxyribonucleotide-deoxyribonucleotide (dN-dN-N-dN-dN-dN) motif. The guide nucleic acid comprises a spacer sequence. The spacer sequence comprises at least one deoxyribonucleotide. The spacer sequence can comprise one to ten deoxyribonucleotides. The spacer sequence can comprise one to nine deoxyribonucleotides. The spacer sequence can comprise one to eight deoxyribonucleotides. The spacer sequence can comprise one to seven deoxyribonucleotides. The spacer sequence can comprise one to six deoxyribonucleotides. The spacer sequence can comprise one to five deoxyribonucleotides. The spacer sequence can comprise one to four deoxyribonucleotides. The spacer sequence can comprise one to three deoxyribonucleotides. The spacer sequence can comprise one deoxyribonucleotide. The spacer sequence can comprise two deoxyribonucleotides. The spacer sequence can comprise three deoxyribonucleotides. The spacer sequence can comprise four deoxyribonucleotides. The spacer sequence can comprise five deoxyribonucleotides. The spacer sequence can comprise six deoxyribonucleotides. The spacer sequence can comprise seven deoxyribonucleotides. The spacer sequence can comprise eight deoxyribonucleotides. The spacer sequence can comprise nine deoxyribonucleotides. The spacer sequence can comprise ten deoxyribonucleotides.

The locations where the ribonucleotide can be replaced with the deoxyribonucleotide can comprise, from the 5′ end of the spacer sequence, position 3, position 4, position 5, position 6, position 7, position 8, position 9, position 10, position 11, position 12, position 13, position 14, position 15, position 16, position 17, position 18, position 19, or position 20. In some instances, the deoxyribonucleotide is located on position 3, 4, 6, 7 and/or 8 from the 5′ end of the spacer sequence. The spacer sequence can comprise deoxyribonucleotides on positions 3, 4, 6 and 7 from the 5′ end of the spacer sequence. The spacer sequence can comprise deoxyribonucleotides on positions 3 and 4 from the 5′ end of the spacer sequence. The spacer sequence can comprise deoxyribonucleotides on positions 6 and 7 from the 5′ end of the spacer sequence. The spacer sequence can comprise deoxyribonucleotides on positions 3, 4, 6, 7 and 8 from the 5′ end of the spacer sequence. The spacer sequence can comprise deoxyribonucleotides on positions 4, 5, 6, 7, 9, 10, 13, and 14 from the 5′ end of the spacer sequence. The spacer sequence can comprise deoxyribonucleotides on positions 3, 4, 5, 6 and 7 from the 5′ end of the spacer sequence. The spacer sequence can comprise deoxyribonucleotides on positions 3, 4, 6 and 7 from the 5′ end of the spacer sequence. The spacer sequence can comprise deoxyribonucleotides on positions 3, 4, 6, 7, 8 and 9 from the 5′ end of the spacer sequence.

The spacer sequence disclosed herein can comprise at least one modified ribonucleotide. For example, the ribonucleotide can be modified at 2′ hydroxyl group to be covalently linked to a methyl group (i.e., 2′-O-methyl). The spacer sequence can comprise one to three modified ribonucleotides. The spacer sequence can comprise one to two modified ribonucleotides. The spacer sequence can comprise one modified ribonucleotide. The spacer sequence can comprise two modified ribonucleotides. The spacer sequence can comprise three modified ribonucleotides. In some instances, the spacer sequence does not have modified ribonucleotide. The spacer sequence can have only unmodified ribonucleotides. The spacer sequence can have only unmodified ribonucleotides and 2′-deoxyribonucleotides. The modified ribonucleotide can be located on the 5′ end of the spacer sequence. The modified ribonucleotide can be located on position 1, 2, and 3 from the 5′ end of the spacer sequence. The modified ribonucleotide can be located on position 1, 3, and 4 from the 5′ end of the spacer sequence. The modified ribonucleotide can be located on position 1 and 2 from the 5′ end of the spacer sequence. The modified ribonucleotide can be located on position 1 and 3 from the 5′ end of the spacer sequence. The modified ribonucleotide can be located on position 2 and 3 from the 5′ end of the spacer sequence. The modified ribonucleotide can be located on position 3 and 4 from the 5′ end of the spacer sequence. The modified ribonucleotide can be located on position 1 from the 5′ end of the spacer sequence.

In some instances, the spacer sequence has three modified ribonucleotides located on position 1, 2, and 3 from the 5′ end of the spacer sequence. The spacer sequence can have three modified ribonucleotides located on position 1, 3, and 4 from the 5′ end of the spacer sequence. The spacer sequence can have two modified ribonucleotides located on position 1 and 2 from the 5′ end of the spacer sequence. The spacer sequence can have two modified ribonucleotides located on position 1 and 3 from the 5′ end of the spacer sequence. The spacer sequence can have two modified ribonucleotides located on position 2 and 3 from the 5′ end of the spacer sequence. The spacer sequence can have two modified ribonucleotides located on position 3 and 4 from the 5′ end of the spacer sequence. The spacer sequence can have one modified ribonucleotide located on position 1 from the 5′ end of the spacer sequence.

In some instances, the spacer sequence comprises five deoxyribonucleotides and two 2′-OMe ribonucleotides. The spacer sequence can comprise five deoxyribonucleotides located on positions 3, 4, 5, 6, and 7 from the 5′ end of the spacer sequence and two 2′-OMe ribonucleotides located on positions 1 and 2 from the 5′ end of the spacer sequence. The spacer can comprise eight deoxyribonucleotides located on positions 4, 5, 6, 7, 9, 10, 13, and 14 from the 5′ end of the spacer sequence and three 2′-OMe ribonucleotides located on positions 1, 2, and 3 from the 5′ end of the spacer sequence. The spacer can comprise four deoxyribonucleotides located on positions 3, 4, 6, and 7 from the 5′ end of the spacer sequence and two 2′-OMe ribonucleotides located on positions 1 and 2 from the 5′ end of the spacer sequence. The spacer can comprise five deoxyribonucleotides located on positions 3, 4, 6, 7, and 8 from the 5′ end of the spacer sequence and two 2′-OMe ribonucleotides located on positions 1 and 2 from the 5′ end of the spacer sequence.

The spacer sequence can further comprise a phosphorothioate backbone modification (PS). The spacer sequence can comprise at least one phosphorothioate backbone modification. The spacer sequence can comprise at least two phosphorothioate backbone modifications. The spacer sequence can comprise at least three phosphorothioate backbone modifications. The spacer sequence can comprise two or three phosphorothioate backbone modifications. The nucleotide residue of the 5′ terminal of the spacer can comprise a phosphorothioate backbone modification. The phosphorothioate backbone modification can be between position 1 and position 2 from the 5′ end of the spacer sequence. The phosphorothioate backbone modification can be between position 2 and position 3 from the 5′ end of the spacer sequence. The phosphorothioate backbone modification can be between position 3 and position 4 from the 5′ end of the spacer sequence. The spacer sequence can comprise three phosphorothioate backbone modifications and the phosphorothioate backbone modifications are located between position 1 and 2, between position 2 and 3, and between position 3 and 4 from the 5′ end of the spacer sequence. The spacer sequence can comprise two phosphorothioate backbone modifications and the phosphorothioate backbone modifications are located between position 1 and 2 and between position 2 and 3 from the 5′ end of the spacer sequence.

The spacer sequence can comprise a (2′-OMe)PS(2′-OMe)PS(DNA)PS(DNA)(RNA)(DNA)(DNA)(DNA) motif. The spacer sequence can comprise a (2′-OMe)PS(2′-OMe)PS(DNA)PS(DNA)(RNA)(DNA)(DNA) motif. The spacer sequence can comprise a (2′-OMe)PS(2′-OMe)PS(DNA)PS(DNA)(DNA)(DNA)(DNA) motif. The spacer sequence can comprise a (2′-OMe)PS(2′-OMe)PS(DNA)PS(DNA)(RNA)(DNA)(DNA)(DNA)(DNA) motif. As used herein, “2-OMe” refers to a modified RNA whose 2′ hydroxyl group of the ribose of the RNA covalently linked to a methyl group; “RNA” refers to an unmodified RNA; “DNA” refers to an unmodified DNA.

The spacer sequence can comprise or consist of SEQ ID NO: 30 or 31. The spacer sequence can comprise or consist of a sequence with at least about 80%, about 85%, about 90%, about 95%, about 96%, about 97%, about 98%, about 99%, about 99.5%, about 100% sequence identity or sequence similarity of SEQ ID NO: 30 or 31. The spacer sequence can comprise or consist of SEQ ID NO: 28 or 29. The spacer sequence can comprise or consist of a sequence with at least about 80%, about 85%, about 90%, about 95%, about 96%, about 97%, about 98%, about 99%, about 99.5%, about 100% sequence identity or sequence similarity of SEQ ID NO: 28 or 29. The spacer sequence can comprise or consist of SEQ ID NO: 11 or 12. The spacer sequence can comprise or consist of a sequence with at least about 80%, about 85%, about 90%, about 95%, about 96%, about 97%, about 98%, about 99%, about 99.5%, about 100% sequence identity or sequence similarity of SEQ ID NO: 11 or 12. The spacer sequence can comprise or consist of SEQ ID NO: 26 or 27. The spacer sequence can comprise or consist of a sequence with at least about 80%, about 85%, about 90%, about 95%, about 96%, about 97%, about 98%, about 99%, about 99.5%, about 100% sequence identity or sequence similarity of SEQ ID NO: 26 or 27. The spacer sequence can comprise or consist of SEQ ID NO: 41. The spacer sequence can comprise or consist of a sequence with at least about 80%, about 85%, about 90%, about 95%, about 96%, about 97%, about 98%, about 99%, about 99.5%, about 100% sequence identity or sequence similarity of SEQ ID NO: 41.

The guide nucleic acid comprising the spacer sequence disclosed herein can comprise or consist of SEQ ID NOs: 110, 111, 112, or 113. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NOs: 110, 111, 112, or 113. The guide nucleic acid comprising the spacer sequence disclosed herein can comprise or consist of SEQ ID NOs: 106, 107, 108, or 109. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NOs: 106, 107, 108, or 109. The guide nucleic acid comprising the spacer sequence disclosed herein can comprise or consist of SEQ ID NOs: 79, 80, 81, or 82. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NOs: 79, 80, 81, or 82. The guide nucleic acid comprising the spacer sequence disclosed herein can comprise or consist of SEQ ID NOs: 102, 103, 104, or 105. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NOs: 102, 103, 104, or 105. The guide nucleic acid comprising the spacer sequence disclosed herein can comprise or consist of SEQ ID NOs: 79, 80, or 82. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NOs: 79, 80, or 82. The guide nucleic acid comprising the spacer sequence disclosed herein can comprise or consist of SEQ ID NO: 132. The guide nucleic acid comprising the spacer sequence disclosed herein can comprise or consist of a sequence with at least about 80%, about 85%, about 90%, about 95%, about 96%, about 97%, about 98%, about 99%, about 99.5%, about 100% sequence identity or sequence similarity of SEQ ID NO: 132.

A guide nucleic acid as disclosed herein can be a single guide nucleic acid comprising a spacer sequence at the 5′-end and a tracrRNA at the 3′-end. The tracrRNA may be modified. For example, some of the ribonucleotides of the tracrRNA can have one or more 2′-OMe modifications. A tracr nucleic acid (e.g., tracrRNA) can have a length of from about 50 nucleotides to about 150 nucleotides.

A tracrRNA sequence can comprise more than one duplexed region (e.g., hairpin, hybridized region). A tracrRNA sequence can comprise two duplexed regions. A tracrRNA may comprise a secondary structure. A tracrRNA may contain more than one secondary structure. A tracrRNA sequence may comprise a first secondary structure and a second secondary structure and a first secondary structure comprises more nucleotides than a second secondary structure. A tracrRNA may comprise a first secondary structure, a second secondary structure, and a third secondary structure and said first secondary structure comprises less nucleotides than said second secondary structure and said second secondary structure comprises more nucleotides than said third secondary structure. The number of secondary structures and corresponding nucleotide lengths is not particularly limited.

Naturally occurring Type V CRISPR systems, unlike Type II CRISPR systems, do not require a tracrRNA for crRNA maturation and cleavage of a target nucleic acid. The tracrRNA can be modified, for example, 2-OMe modification. The tracrRNA can be a sequence of GUUUUAGAGCUAGAAAUAGCAAGUUAAAAUAAGGCUAGUCCGUUAUCAACUUGA AAAAGUGGCACCGAGUCGGUGCUsususu. The tracrRNA can be a sequence of gUUUUAGagcuaGaaauagcaaGUUaAaAuAaggcuaGUccGUUAucAAcuuGaaaaagugGcaccgaguc ggugcusususu.

A target nucleic acid can comprise DNA, RNA, or combinations thereof and can be a double-stranded nucleic acid or a single-stranded nucleic acid. A spacer sequence can hybridize to a target nucleic acid that is located 5′ or 3′ of a protospacer adjacent motif (PAM), depending upon the particular gene editor protein to be used. A PAM can vary depending upon the gene editor protein to be used. For example, when using the Cas9 from S. pyogenes, the PAM can be a sequence in the target nucleic acid that comprises the sequence 5′-NRR-3′, wherein R can be either A or G, wherein N is any nucleotide, and N is immediately 3′ of the target nucleic acid sequence targeted by the targeting region sequence. A gene editor protein can be modified such that a PAM may be different compared to a PAM for an unmodified gene editor protein. For example, when using Cas9 from S. pyogenes, the Cas9 may be modified such that the PAM no longer comprises the sequence 5′-NRR-3′, but instead comprises the sequence 5′-NNR-3′, wherein R can be either A or G, wherein N is any nucleotide, and N is immediately 3′ of the target nucleic acid sequence targeted by the spacer sequence. Other gene editor proteins may recognize other PAMs and one of skill in the art is able to determine the PAM for any particular gene editor protein. For example, Cpf1 from Francisella novicida was identified as having a 5′-TTN-3′ PAM (Zetsche et al. (Cell; 163(3):759-71(2015))), but this was unable to support site specific cleavage of a target nucleic acid in vivo. Given the similarity in the guide sequence between Francisella novicida and other Cpf1 proteins, such as the Cpf1 from Acidaminocccus sp BV3L6, which utilize a 5′-TTTN-3′ PAM, it is more likely that the Francisella novicida Cpf1 protein recognizes and cleaves a site on a target nucleic acid proximal to a 5′-TTTN-3′ PAM with greater specificity and activity than a site on a target nucleic acid proximal to the truncated 5′-TTN-3′ PAM misidentified by Zetsche et al. The polynucleotides and CRISPR systems described in the present application may be used with a Cpf1 protein (e.g., from Francisella novicida) directed to a site on a target nucleic acid proximal to a 5′-TTTN-3′ PAM.

A target nucleic acid sequence can be 20 nucleotides. A target nucleic acid can be less than 20 nucleotides. A target nucleic acid can be at least 5, 10, 15, 16, 17, 18, 19, 20, 21, 22, 23, 24, 25, 30 or more nucleotides. A target nucleotide can comprise ranges of nucleotides between about 5-30, and ranges between. The selection of a specific PAMs is within the knowledge of those of skill in the art based on the particular gene editor protein to be used in a given instance.

The spacer sequence of the present disclosure comprising DNA and RNA on the same strand can be chemically synthesized. Chemical synthesis of polynucleotides is well understood by one of ordinary skill in the art. Chemical synthesis of polynucleotides of the present disclosure can be conducted in solution or on a solid support. Solid phase synthesis is the preferred method of making the guide RNA for early evaluation.

The guide nucleic acid containing DNA may provide the advantage of increased specificity of targeting target nucleic acids such as DNA. Without being bound by any specific theory, spacer sequences comprising DNA in specific regions as discussed herein present the advantage of reducing off-target binding due to localized structural perturbation in the spacer leading to less off-target site-binding.

The spacer sequences of the present disclosure can also comprise modifications that, for example, increase stability of the polynucleotide. Such modifications may include phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and amino alkylphosphoramidates, phosphorodiamidates, thionophosphoramidates, thiono alkylpho sphonates, thionoalkylpho sphotriesters, selenophosphates, and boranophosphates having normal 3′-5′ linkages, 2-5′ linked analogs, and those having inverted polarity wherein one or more internucleotide linkages is a 3′ to 3′, a 5′ to 5′ or a 2′ to 2′ linkage. Suitable nucleic acid-targeting polynucleotides having inverted polarity can comprise a single 3′ to 3′ linkage at the 3′-most internucleotide linkage (i.e., a single inverted nucleoside residue in which the nucleobase is missing or has a hydroxyl group in place thereof). Various salts (e.g., potassium chloride or sodium chloride), mixed salts, and free acid forms can also be included.

The deoxyribose or ribose (i.e., sugar moieties) on the deoxynucleotide or nucleotide of the spacer sequences can be modified. The examples of modified sugar moieties include, but are not limited to, 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino). Examples of such analogs and/or modified residues include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, methyl phosphonates, chiral-methyl phosphonates, 2′-O-methyl ribonucleotides, peptide-nucleic acids (PNAs), and the like.

The spacer sequences of the present disclosure may also contain other nucleic acids, or nucleic acid analogues. An example of a nucleic acid analogue is peptide nucleic acid (PNA).

### Linkers

Gene editing systems provided herein can comprise linkers that connect one or more components of the gene editing systems. The linkers may be used to link any of the protein or protein domains described herein. The linker may be as simple as a covalent bond, or it may be a polymeric linker many atoms in length. The linker can be a polypeptide or based on amino acids. The linker may not be peptide-like. The linker can be carbon bond, disulfide bond, carbon-heteroatom bond, etc. The linker can be a carbon-nitrogen bond of an amide linkage. The linker can be a cyclic or acyclic, substituted or unsubstituted, branched or unbranched aliphatic or heteroaliphatic linker. The linker can be polymeric (e.g., polyethylene, polyethylene glycol, polyamide, polyester, etc.). The linker can comprise a monomer, dimer, or polymer of aminoalkanoic acid. The linker can comprise an aminoalkanoic acid (e.g., glycine, ethanoic acid, alanine, beta-alanine, 3-aminopropanoic acid, 4-aminobutanoic acid, 5-pentanoic acid, etc.). The linker can comprise a monomer, dimer, or polymer of aminohexanoic acid (Ahx). The linker can be based on a carbocyclic moiety (e.g., cyclopentane, cyclohexane). The linker can comprise a polyethylene glycol moiety (PEG). The linker can comprise amino acids. The linker can comprise a peptide. The linker can comprise an aryl or heteroaryl moiety. The linker can be based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

The linker can be an amino acid or a plurality of amino acids (e.g., a peptide or protein). The linker can be a bond (e.g., a covalent bond), an organic molecule, group, polymer, or chemical moiety. The linker can be 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. A linker can comprise the amino acid sequence SGSETPGTSESATPES, which may also be referred to as the XTEN linker. A linker can comprise the amino acid sequence SGGS. A linker can comprise (SGGS)n, (GGGS)n, (GGGGS)n, (G)n, (EAAAK)n, (GGS)n, SGSETPGTSESATPES, or (XP)n motif, or a combination of any of these, wherein n is independently an integer between 1 and 30, and wherein X is any amino acid. In some instances, n is 1, 2, 3, 4, 5, 6, 7, 8, 9, 10, 11, 12, 13, 14, or 15. A linker can comprise SGSETPGTSESATPES, SGGSSGSETPGTSESATPESSGGS. A linker can comprise SGGSSGGSSGSETPGTSESATPESSGGSSGGS. A linker can comprise GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS. The linker can be 24 amino acids in length. The linker can comprise the amino acid sequence SGGSSGGSSGSETPGTSESATPES. The linker can be 40 amino acids in length. The linker can comprise the amino acid sequence SGGSSGGSSGSETPGTSESATPESSGGSSGGSSGGSSGGS. The linker can be 64 amino acids in length. The linker can comprise the amino acid sequence SGGSSGGSSGSETPGTSESATPESSGGSSGGSSGGSSGGSSGSETPGTSESATPESSGGSSG GS. The linker can be 92 amino acids in length. The linker can comprise the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS. It should be appreciated that any of the linkers provided herein may be used to link a first adenosine deaminase and a second adenosine deaminase; a deaminase (e.g., a first or a second adenosine deaminase) and a gene editor protein; a gene editor protein and an NLS; or a deaminase (e.g., a first or a second adenosine deaminase) and an NLS.

Various linker lengths and flexibilities between a deaminase (e.g., an engineered ecTadA) and a gene editor protein (e.g., a Cas9 domain), and/or between a first adenosine deaminase and a second adenosine deaminase can be employed (e.g., ranging from very flexible linkers of the form (GGGGS)n, (GGGGS)n, and (G)n to more rigid linkers of the form (EAAAK)n, (SGGS)n, and (XP)n) in order to achieve the optimal length for deaminase activity for the specific application. In some instances, n is any integer between 3 and 30. In some instances, n is 1, 2, 3, 4, 5, 6, 7, 8, 9, 10, 11, 12, 13, 14, or 15. The linker can comprise a (GGS)n motif, wherein n is 1, 3, or 7.

Additional linkers can be found in PCT Application Publication NO. WO2021/207712, which is hereby incorporated by reference in its entirety.

### ANGPTL3 Gene

The target gene for modification using the systems and methods disclosed herein can be a gene encoding ANGPTL3. ANGPTL3 has been associated with diseases and disorders such as, but not limited to, Arteriosclerosis, Atherosclerosis, Cardiovascular Diseases, Coronary heart disease, Diabetes, Diabetes Mellitus, Non-Insulin-Dependent Diabetes Mellitus, Fatty Liver, Hyperinsulinism, Hyperlipidemia, Hypertriglyceridemia, Hypobetalipoproteinemias, Inflammation, Insulin Resistance, Metabolic Diseases, Obesity, Malignant neoplasm of mouth, Lipid Metabolism Disorders, Lip and Oral Cavity Carcinoma, Dyslipidemias, Metabolic Syndrome X, Hypotriglyceridemia, Opitz trigonocephaly syndrome, Ischemic stroke, Hypertriglyceridemia result, Hypobetalipoproteinemia Familial 2, Familial hypobetalipoproteinemia, and Ischemic Cerebrovascular Accident. Editing the ANGPTL3 gene using any of the methods described herein may be used to treat, prevent and/or mitigate the symptoms of the diseases and disorders described herein.

The ANGPTL3 gene encodes the Angiopoietin-Like 3 protein, which is a determinant factor of high-density lipoprotein (HDL) level in human. It positively correlates with plasma triglyceride and HDL cholesterol. The activity of ANGPTL3 is expressed predominantly in the liver. ANGPTL3 is associated with Dyslipidemias. Dyslipidemias is a genetic disease characterized by elevated level of lipids in the blood that contributes to the development of clogged arteries (atherosclerosis). These lipids include plasma cholesterol, triglycerides, or high-density lipoprotein. Dyslipidemia increases the risk of heart attacks, stroke, or other circulatory concerns. Current management includes lifestyle changes such as exercise and dietary modifications as well as use of lipid-lowering drugs such as statins. Non-statin lipid-lowering drugs include bile acid sequestrants, cholesterol absorption inhibitors, drugs for homozygous familial hypercholesteremia, fibrates, nicotinic acid, omega-3 fatty acids and/or combination products. Treatment options usually depend on the specific lipid abnormality, although different lipid abnormalities often coexist. Treatment of children is more challenging as dietary changes may be difficult to implement and lipid-lowering therapies have not been proven effective.

ANGPTL3 is also known to cause hypobetalipoproteinemia. Hypobetalipoproteinemia is an inherited disease (autosomal recessive) that affects between 1 in 1000 and 1 in 3000 people worldwide. Common symptoms of hypobetalipoproteinemia include plasma levels of LDL cholesterol or apolipoprotein B below the 5th percentile which impairs the body's ability to absorb and transport fats and can lead to retinal degeneration, neuropathy, coagulopathy, or abnormal buildup of fats in the liver called hepatic steatosis. In severely affected patients, hepatic steatosis may progress to chronic liver disease (cirrhosis). Current treatment of hypobetalipoproteinemia includes severe restriction of long-chain fatty acids to 15 grams per day to improve fat absorption. In infants with hypobetalipoproteinemia, brief supplementation with medium-chain triglycerides may be effective but amount must be closely monitored to avoid liver toxicity. Another option for treating hypobetalipoproteinemia is administration high doses of vitamin E to prevent neurologic complications. Alternatively, vitamin A (10,000-25,000 IU/d) supplementation may be effective if an elevated prothrombin time suggests vitamin K depletion.

The target tissue for the systems and methods described herein can be liver tissue. The target gene can be ANGPTL3 which may also be referred to as Angiopoietin 5, ANGPT5, ANG-5, Angiopoietin-Like Protein 3, Angiopoietin-5, FHBL2, and ANL3. ANGPTL3 has a cytogenetic location of lp31.3 and the genomic coordinate are on Chromosome 1 on the forward strand at position 62,597,487-62,606,159.

Loss-of-function mutations that may be made in ANGPTL3 gene using the methods and systems described herein are also provided, including, but are not limited to premature stop codons, destabilizing mutations, altering splicing, etc.

The gene editing system of the present disclosure may reduce expression of functional ANGPTL3 protein encoded by the ANGPTL3 gene in the cell. The modification can reduce expression of functional ANGPTL3 protein encoded by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100%. The modification can reduce expression of functional ANGPTL3 protein encoded by the ANGPTL3 gene in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold. The modification can abolish expression of functional ANGPTL3 protein encoded by the ANGPTL3 gene in the cell.

A splice site disruption generated by a gene editing system disclosed herein can result in the inclusion of intronic sequences in messenger RNA (mRNA) encoded by the ANGPTL3 gene. The splice site disruption can generate a nonsense, frameshift, or an in-frame indel mutation that result in premature stop codons or in insertion/deletion of amino acids that disrupt protein activity. The splice site disruption can generate exclusion of exonic sequences. The splice site disruption can generate exclusion of exonic sequences that results in nonsense, frameshift, or in-frame indel mutations in the ANGPTL3 transcript. Canonical splice donors comprise the DNA sequence GT on the sense strand, whereas canonical splice acceptors comprise the DNA sequence AG. Alteration of the sequence disrupts normal splicing. Splice donors can be disrupted by adenine base editing of the complementary base in the second position in the antisense strand (GT to GC), and splice acceptors can be disrupted by adenine base editing of the first position in the sense strand (AG to GG).

A gene editing system provided herein can affect an A●T to G●C alteration in a ANGPTL3 gene when contacted with the ANGPTL3 gene. The A●T to G●C alteration can be at a splice donor site of the ANGPTL3 gene. The A●T to G●C alteration can be at a splice acceptor site of the ANTPTL3 gene. The A●T to G●C alteration can result in an aberrant ANGPTL3 transcript encoded by the ANGPTL3 gene. The A●T to G●C alteration can result in a non-functional ANGPTL3 polypeptide encoded by the ANGPTL3 gene. The A●T to G●C alteration can be at a 5′ end of a splice donor site of an intron 6 of the ANGPLT3 gene.

The nucleotide sequence of human ANGPTL3 is provided, for example, in NG_028169.1, which is incorporated herein in its entirety. The protein sequence of human ANGPTL3 is provided, for example, AAD34156.1, which is incorporated herein in its entirety.

Mouse, rat, and monkey ANGPTL3 nucleic acid sequences have been deposited; see, e.g., Ensembl accession number ENSMUSG00000028553, ENSRNOG00000008638, and ENSMFAG00000007083 respectively, each of which sequences are incorporated herein its entirety.

The polypeptide and coding nucleic acid sequences of ANGPTL3 and of other members of the family of human origin and those of a number of animals are publicly available, e.g., from the NCBI website or ENSEMBL website. Examples include, but are not limited to the following sequences, each of which sequences are incorporated herein in their entireties:

NG_028169.1 Human angiopoietin like 3 (ANGPTL3), RefSeqGene on chromosome 1

(SEQ ID NO: 1)
AATGACAAACTGAAAAAATCTATTGTTTGTTATATATATAACAAAGAATTAGTATCC
ACAATATGTAAATAATTCCTAAAATTAGTCAGAAAGAGACAAACTTAAAAAGAGGG
TAACAAGGAGGGGAGCAAATTATGTACATAACCAGATGATTCGCAAAGACGGCAAC
AGAGATGGCCAGCAAAACAAACTAGATATATACTTGTCTATTAGATTTATCAACATT
TTTTGCCTTTTTCATTAAAAGCATTTGTAAAAGGATATAGGAAAAGAGGAACTCTCA
TATACTCCTGGCAGGGATGTAAATTGGTACAACCCTTTTGAAGGACAATCTGACAAA
AGCAATCGTAAGTTACAAGTCAACATCTATGAATGTATATGAAAATATTTATATACA
TACATCACCACCATAAAAGCATTTTCTATACATACTGTTTATAATTAGAAAATTGGA
AACAAATGATTAAAAGGGGGCTGATTAAATTAAGGTTCATCTATATAACAGGATTAT
GCAGCTATTAAAAAGGACGTGGTAACTCTATAGACATTCATAGGAAAATAAATTTTA
AAATACTAAGATCCTGAATGATATATATATCATGAGCTATTATACATAACAAGATCC
CACTTGTGTTATAAAAAATTATGTTTAGTCATTCAAAGGGTCTGGTATGATAGACCC
AAAATGTTAATAGAGTCGAGATTTTTATTTTTTATAGGTTTTTGAAATACCTGAATTT
TCACAATAAGTACTTTGCACATTAAAAATCTTAGCTGGGCATGGTGGCTCACGCTTG
TAATCCCAGCACTCTGGGAGGCCAAGGTAGGCAGATCACCTGAGGTCAGGAGTTCG
AGACCAGTCTGGCCAACATGGTGAAACCCCGTCTCTACTAAAAATACAAAAATTAG
CCAGGCTTGGTTGGGGGTGCCTGTAATTCCAGATACTCGGGAAGCTGAGACAGGAG
AATCGCTTGAACCCAGGAGGGGGAAGTTGCAGTGAGCTGAGATCACGACGCTGCAC
TCCAGCCTGGGCAACAAAGAGCAAAACTCCGTCTCAAAAATAAATAAAGAAAAAAT
CTTTACATGTCCAAAGATACGGCTGTTCAACTAAAAAATATATATGTATAAAACTTA
ACATGTTAATAGTGAACACACAAAACAGTAAGATAGATAAAATTATTCCTTCAAAG
CTCACTTAACCTCTGGATCTACACTGTCCAAAAAGACGGTCTAATGAGACAATTGAG
CACTTGATAGGTGAGTGGTTCTAACTGAGATATGTTCTCAGTATAAAACATACAATA
GGATCTTCCTATACAACATTAATTAAAAAACAAACTATTGTAGTTAAAAAGGAAAAA
ATTAGAGATACTATGTAAAAAAGAGCCAAAATACCTTGTATTTTATTTGAAAGACAT
ATCTCCATAAGATTACACAACCTCGTGTAGGATAAAGGACTTTGCTTTGCTTGGAAT
TTAAACAATTTAGGCTCTTAAATGTCCTAAAATTCTCTGTAGCTAAGAAATTTTTATA
TTGGTTCCTAGGAACTAGGAATCCTTAAATTAGGCCCTACATTTGCTTACAAGTTTAT
TTTCCTTGGCATAAAATTTTTTAGTTTTTACATTACTGGTTATATTTGATCAGGGTTCT
ATTTAAATAGGCACAAGTTCAAGCAAAGATCAGATTCTGCTTTTAGCAGTGTGTACT
CAGACAGGAAGTATTAAAAGGCAGGCAGAAAATCCTTTATAAAATTACTACTTTCA
ATGCATTTTCCCACGTTGAAATGCTTCTGCAGTTTATAATTAGGCAAATTACTTTAAT
TATAATCAATAATGCTGTTCAAATTACTATAAGAATTATACAGATATTTATACCAAG
AGACAATATACTAGAAACCAAGACTACGTGACCATTACCTCTACTCTGTCAGTGTTA
TTTGTGAGAAATTGCACAAATTTTGCAAAAAGTGTTAGTATCCTACTACAGTAGGAT
ATAATATAGAAGGAAATAATTTCATAAAGCCTGTCTTTGGTACTAGTGCTCAGTTAC
TTTCATTAACTAAAAAAGGGGCTACTCTTCAAATTCCCTTCTCTAAAAAGAATGTAC
TATATCAAAAGGGGGTAAACACTACTACGTATACATTCTGCACTTAGAAATCCCTAT
ATGTTGATTTTATCATTCTCTTATTCAATAAATATTGTTTCTACAATGTGTAAGGCATT
ACTGTACTAAAGCATTATAAGGAATATAAGTTAAAAACACATACAAATCTTGCCAAT
CAACAGCTTATAGTGTAATAGGGGAGAGAAGCTGGCCCATCTATATTCTCCCTCAAC
TAGCAAGTGGATGAAATATCAGGGTCAATAGTTATAAGCCACAAAAGCTGACAGCT
TAATTAAGAGAAGTTTTGAAATATGTATTTCATGACCAATAATTACAACTGTAACTTT
TCTATTTAAAGAAGGAGAAAATTTGAATTTCTTCTCTAGCTCAACATACACTTCTATA
ATTCCATTACATGAACCAGAGTAAAGGGTAAGATGGAAATGAAGAATATTTTCTTAC
CCTTTTGTGGTTCTATATTGGACACTTAAAAATCATACACAACCTAATCAAAAGATG
TAATTCTTTAAAAAGGTACGAGACCAAAATTCAGAAAATCTAGACTATAACAAAATT
TCTCAATTTACATTATCTTAATATGCAATTAATTTTCACCAGTAAAATACTATAGTAT
GGGTACAAATGCATTGATTAGTTCTAATTACAAAAATGGCTAATATATAATACTGTG
TAGTGTTTATGATACATCAGATAATGTTCTAAGTGCTCTGAAAATATAAACTTTTAAT
CTTTTATACGACCCTATAAAATAGGTGTTATTCTCACTGGAGAGATGAGAAAACAGG
GGTTCAGAGATGTGAAGTAATTTGACCAAAGGTCACAAAGCTGAAGAATATGAAAT
CCGGGATTCTGATTCAGGCAGTCTTATTCCAGAATCATGCTCTTAACCACTATGGAA
TACTGCCTCTACTGTAACTATTATACCCAAAACCCTTAATCCTAAGTCATCAAAAGG
AAGAGCCTCTATTTTACACAATGAAGAGGCATTTCTAAGAATAGAAATTTAGGGACG
AGCACAGTGGCTTACTCCTTTAATATCAGCACTTTGAGAGGCTGATATGGGAGGTTC
ACTTGAAGTCAGGAGTTCAAGGTCAGCTTGGGCAACATAGTGAAACACAGTCTCTAC
AAAATATTTAAAAATTAGCTGGGTGTGGTGGCATGCATCTATAGTCTCAGCTACTTG
GGAGACAGAGGGAGGAGGCTTGCTCGAGCCCAGGAGTTCGTGGCTATAGTGAGCTA
TGATCATGCCACTGCACTCCAGCCTGGACAACAGAGCAAGACCCTGTCTCTAAAAAA
GAAAAGAAATTTGGAAATGGTTTATTTTGTATTAACAATTTATAATTTACACTGAAA
TTTATTATGATAAAACTTTTCCCTGTGTTAAAAAGCTATTAACTTTATGAAAAATTTC
TTTTAGGTAAGGTTGATTATATATACCCACACACATACACAGGTTAAAAGTTAGTTT
CATGTGACATAATAACTAGCATTTTGAGCACTACCTGTTTGCCCAGCACTGTTCTAA
GTGCTCTACATGTATTATTGTTAAATTATCATAACACTATGAATTATGTACTATAATT
ACCCCAGCTTTACAGATGAGGAGACTAATCCATGGGGAGGTTAAGTAACTTGTCCAA
GGCCAGACAGCTAGAGCCGGCTTTTGGACCCACACCACAGTCTGACTCCAGCACCCA
TATTCTTAACAATTTCACCATATTAATATGTCAAGATTAAGCAGTTTTAAAGGATGCT
ATTTTCTCACAAATTTCTTAATATGAACACTCAATAAGAATAATCACTAATATAAGC
ATTTAGTATTTTTTTAACACTAAGTTGGAAGCATAGTGGAACATTTATTTTTAGAAAT
ATTATTAATTGGCTGGGCTCACGCTTGTAATCGGCTGGGCTCATGCCTGTAAATTTTG
GGAGGCCAAGGTAAAAGAATTGCTTGAGCCCAGTATTTCCAGACCAGCATGGGCAA
TACATTAAGACATCATCTTTAAAAAAAAAATGTTATTAATCTCCTCTTTTTGTTAAAT
GTATATTATCAAAATTGTTACTAAGCTAACAAACTTCAGAAAAACTTATGATGGGCA
AGCTGCTTGTGACATTGAAGGTATTTAAGATTCAATTCTAGTTTGGTCCTAGATGACC
ACATATCCATTGTTCCTTCAACGAGCACATGGTAAAGAGCCTAGAACACAGAGACA
CAGAACACAGTGGAGAAAAGGGAGTGAAATGTCTTTAATGACACTTACTATATATG
GGATTTTGTGACAATATACAAGGATGGTTAAGACATATAAGGTGATGCAAAAAAAC
ATATTAACAATTATAGTGACAAAAAATGAGGAGCATATAATTATACATTGATTTATA
CAGAGTACCAGAGGAACACAGCATTGAGAGCCGTAACACCACCTGAGGGAGTGGAG
AAAGGCTTCAGAGAGAAAGTGTTTTTTGGAATGGATCACTGTTTCCAAAAGAACTAA
AGTACAGTTTGAGAAATGCATACTTAATTCATTACTTTTTTCCCCTCAACTTTAATAA
TAAATTTACCCAACAAAAAAGTTTATTTTTGACTTGTAAATCTCTTAAAATCATAAAA
AAGTAAAATTAGCTTTTAAAAACAGGTAGTCACCATAGCATTGAATGTGTAGTTTAT
AATACAGCAAAGTTAAATACAATTTCAAATTACCTATTAAGTTAGTTGCTCATTTCTT
TGATTTCATTTAGCATTGATCTAACTCAATGTGGAAGAAGGTTACATTCGTGCAAGTT
AACACGGCTTAATGATTAACTATGTTCACCTACCAACCTTACCTTTTCTGGGCAAATA
TTGGTATATATAGAGTTAAGAAGTCTAGGTCTGCTTCCAGAAGAAAACAGTTCCACG
TTGCTTGAAATTGAAAATCAAGATAAAAATGTTCACAATTAAGCTCCTTCTTTTTATT
GTTCCTCTAGTTATTTCCTCCAGAATTGATCAAGACAATTCATCATTTGATTCTCTAT
CTCCAGAGCCAAAATCAAGATTTGCTATGTTAGACGATGTAAAAATTTTAGCCAATG
GCCTCCTTCAGTTGGGACATGGTCTTAAAGACTTTGTCCATAAGACGAAGGGCCAAA
TTAATGACATATTTCAAAAACTCAACATATTTGATCAGTCTTTTTATGATCTATCGCT
GCAAACCAGTGAAATCAAAGAAGAAGAAAAGGAACTGAGAAGAACTACATATAAA
CTACAAGTCAAAAATGAAGAGGTAAAGAATATGTCACTTGAACTCAACTCAAAACT
TGAAAGCCTCCTAGAAGAAAAAATTCTACTTCAACAAAAAGTGAAATATTTAGAAG
AGCAACTAACTAACTTAATTCAAAATCAACCTGAAACTCCAGAACACCCAGAAGTA
ACTTCACTTAAAGTAAGTAGAAAATAAAGAGGGTTCATGTTTATGTTTTCAATGTGG
ATCTTTTAAAAAAAATATTTCTAAGGCATGCCATTTGAAATACTTTGTTGCATTGTTG
AAATACTTTTTTTTCCAAGAAAAATAATCTCCAGAAAATAAAATTTCCTATTATAATT
TCAAGTTAGTTTTTTGTTTCCCTAATGTTATATATGAAAACACTGAAAATTTGCATTT
TATATGAAAATTACAAATCGGTTAAATTATACAATCTAGAACACTATGTCATTACAC
TATTGTAAATTACTGAAGGTAAGTAAAAAGTTAAAAAAAATTTAAAACTATTCTCCA
GTGTTTAAAACAGATTAAATAATACAGTAAATGGAAAAGATTTATTCATATGAAAAT
ATGCTGGGCTTTTTCTTTTAATTGAAGTTCAGAAAATCAAATTTTAGAGATAGTACAA
TTTAAATAAAATGTTAAGGACAAAAATATGTGCTATTTGAAAGAAGCATACAAGGG
GAAGGAATTGCCAATATTCATTTTTCAAATCCATTATTAGTTTAAAAATTTAGATTAT
GATAGTGTTACAGGAAATTAATAGAAAAGAAAGAGGAAAGCAACTTATAACCAACC
TACTCTCTATATCCAGACTTTTGTAGAAAAACAAGATAATAGCATCAAAGACCTTCT
CCAGACCGTGGAAGACCAATATAAACAATTAAACCAACAGCATAGTCAAATAAAAG
AAATAGAAAATCAGGTAAGTCAGTATTTTAATGGTATGTCCCATCTTTCACACAGGT
CTGTAAAAACACTGAATCCTAAAATTATTTACAAGCTTTAACTGGATCATGAGTAAA
ATTATCACATCAGCATAACTGTTAAAATTGCAGGCTCTGAAGCTAATAAACTACCTG
CATTTAAACCATGGCTCTAAAACTTTGTGTGACCTTGAATAAATTACTTCACCCCTTT
ATCTCTCAGTTTCCTCACATATACTACAAAGATAATAACAGAACTTATAGGATTATT
GTAAGAAAAAAAATTAATTCATAGCAGCCAATGTCATCTTACTAAAATTCAAATTAG
ATCATGTTTCTCTTTGCTCAAAACCACACAATAGCTTTCCATTTCACTCATATTGGCT
CTTTAGACCAAGATTACCCAACCCTTCGTCATCTCACTGACTTCACCTCCTCTACTCT
AGTTATTCTGACCGCTTTACCAGTATTCAAACACATCAAACATACTGCCACCTCAAA
GCCTTTGCCCTTGTTGTTTCCTCTAACTGGAACGCTCTTCTGCCCTGGTATCTACGTG
GCCCACTCTCTGATTTCCCTTAGGGTCGTTATCAAACAAAAAATTCCCAATGAAGAC
TTACAAGGTCACTTAACCAAAAATCACAACCGCCTGGTCCCATCCCTGAAAACTTCT
ACTTCCTTAGCTACTTTTCTCCTGCACACTCACCTTTATTTAACATAACATAAATTTTA
GTTATTTATCTCTTCTATTCCTGCACTAAAATGTAAGCTCTGTGAATACAGGGATTTT
TTCCATTATCTTCATATTTTCCATTATTTGTATATACTCCAGAATATAGAATACTGTAT
GGCACACAGTAGGCATTTCTGTTGAATTAATAAATGTAATGTCATATTCACACAGAA
GCGTGTGCTATGATTATTATTACTTGGATTACTAGAAATAGTGTGCCTCATAATTAAA
GGTCAACATTCAACAATGTAATTAATCTACAATGTAAACATCTGGTGAAGTGACAGA
GGGAAGCACTTGTTTAGAAAAAAGCTATGTCAGAATCCATGTATTCTAATATGCAGT
ACAATAGTTTAAAAATATTAATAATACTCTCAAACAGCTATTCAAGAGGATTCAAAA
AACATAATATAAACTCAGAGAAACTGGTAAACAAAATCATTTTCAAGAGATATAAA
ACAAATATTATTACCAATTTCCACTAAACAAACATAATGTTAGTAGTGCTGCTAAAA
GGTTTTTTATCAACTACTTTTGGTTTCCATACTTTCCTTCTTATGATGTTATTATTCTA
AATTCTTTTCAATTATATCTTTTACTATGATTAAATGAACCTGCTCCCCAAAGCAAAA
TGTTACTATAGTAATATACATTGTGTCTAAAAATAAAAATGTGTGAAGAAACCAAAA
CAATGAATTTCTGAGTTGGAAGAAGAGTTAGATCATTTAACTTTCTCATATTTAAATT
AAAAAAACAAAACTCTAAAAATTTAAGTAACTTTAAGATCACATAGTTACTTAGTAG
AAAAGAGTAATACCCAGCAAGCAAACTTTACAATAGATCCTTTTAAATAAGGTCCTA
GGAAATATCATTCATGCCAGCATCAAAAAACTAACACTAATAATGCAAGATATTATA
TATTCTGCTTTTCTTACTGTCAATGAGAAAAACTATCATTCAATAAATTGCAAACCCA
ACACACTTAAATAAAAATAAAATGTTACTGCTAAACTAACGATAAACTACTGAATAT
ATAGAAAGTAAGCAAACAAACTTGCCAACCTGCCAACATCTACAGATATGTTTACA
GGTCAAAAATTATCAAATTATCAAGAAAGCCTGGTTCAAATTATGTATTATGTCTTT
ATCACAGGTCTGAAGATCAGTAAGACCTAAAACTGAAAATTATTAAACTTAAAATCT
GAACAGAATATCAAATATATTTTATTCATATAAATAAAAGAATACATTACAATATTC
TAAGCAAAGCAGTCTCTACTTTTGGCCTTGCTCTGTTTTCCGACCAATGTCTGCTTTT
TTGCCTTGCTTTATTTTTTTATCTTATTAAATAATGTCCCTGATTAAATATTTTGAGAA
CAGGTAATCTGTACAATCTGAATAACACTGTTTATCTAAATATCAAACACCGTTATA
ACATTATGAACTGAAAGACAAACTGTACTTCTGACATCCTTACTCAGATTTCCCCTA
ATTGTATATTCAGTATCATTTTAAAAAACAGATTTATATTCTTTTATCAGCTCAGAAG
GACTAGTATTCAAGAACCCACAGAAATTTCTCTATCTTCCAAGCCAAGAGCACCAAG
AACTACTCCCTTTCTTCAGTTGAATGAAATAAGAAATGTAAAACATGATGGTAAGAC
ACTTTGGTGGGTTTCCTTCTTGAAGCTATTATTATCAAATTCCCTATTCTTAGGACTTG
TTCTAGACTAAAAGATAGTTAAGAGATATCCATCAAATACAATGTATCAACCTAAAC
TGGATGCTGGGGTTCTTTTTACACCCTATAAAAGACATACCTAAGACAATCAGAGAA
ATACAAATATGGACTTGATTATTAGATAATATAGAAGGTTTATTAATTTTCTTAGATG
TGATCATGGTATTGCAGTTTTAAAGGAGAACAATCTCCTGTTTAAGAGATACATGCT
GAAATATTTACGGAGTTAAAGGTCACTGGACTCCAGACTGGTGATAGAACAAGACT
CTGTCTCTAAAAAATAATTAATTTTTTAAAAGAAAATAGTTTGGTAAGATGATTCTTA
CATTCTTAAATAACACGCCATCTAAGAAAAATGCTTTAACATAAACATTACTGAAAA
AATGCTACATTTGCCACAACTTCATAAAATGTCAAGTGAAATCTCAAGCTCCAAAGA
TATTATTCCTATTACTAAATCTGATGTAATAACATTTTATTGATTCTAGGCATTCCTG
CTGAATGTACCACCATTTATAACAGAGGTGAACATACAAGTGGCATGTATGCCATCA
GACCCAGCAACTCTCAAGTTTTTCATGTCTACTGTGATGTTATATCAGGTAAAACCTG
TCTAAGGAGAATAGACAGTAGTTAGTTCAACTTACTCATTACGTATTAGGAAGATTA
ACCTGGTTATCATTGTTTTATACATATATATATGAAATATATATGAGTATTCGTATAA
ATATAATACTTTTACCTTGTTTATGTATTTACTCAATATTCTCCTTTTCCTCTAAAATA
ATCTGAAGTGACTATTATCAATAAGTTTACTATGCCAAAATTCATTAATTGCCTTTCA
CTTAACTTTTGGGACCATAATAAATAATAAAATGTATTGCCATAACATTAATAAACT
ACCTTACAAAACCACCAATTAAAATCAAACAAACAAAAAAGTGTTATTTACATCTGT
CAACATAAATCTACTAAAAATACATGATTTCATTCATTATATTCAGGTAGTCCATGG
ACATTAATTCAACATCGAATAGATGGATCACAAAACTTCAATGAAACGTGGGAGAA
CTACAAATATGGTTTTGGGAGGCTTGATGGTAAGGGGACTACATTCAATCATTCATT
CACTTGCTAATCTACAAATATTTACTGAGAACCTCTTATGGACCAGGTATTAGGAAA
AGTAGTAACGAACGAGAAGCAGTCTCAGCCTTCATATAATTTATTATCAAACAATTA
CACATTTGTTAGTAAATTACACTTATTACAACTGTTATTATTTGAATTATATTTATCA
CAATTACATGTCTGTTCTTAAATATACTTATCACAATTTAATTCCACGGCTTACAATG
ATCATAACTATAATTATTAAAGACAATTTTGATTAAATGTTATGTCATAAGTAGTAA
CTGTTACAAATAAGCTGTGAAAAGAACCACTCCTAGCATTAGTCACTCTATTCTCTC
ATTAACGTTTTACATATCAATTAATTGGAAGTTAAAAGGACCAGGAAACTCAGACAT
ACAGTATACATTTTAAAATTTCAATTATTTAAATATAATATATAGAATGTATGGCTTA
TAATGAATTAGTTAACTCAATGCAAATTATTCTATTTTGATTACAAATAGTAAAATA
AGCAAGATAAAATAACAGATGTTTAAAATCCAAAAAGCACATACAAAAATCCATGA
ATGATGTCTAAGTACTCACTTATAAAGTAGAAGACATTCATTATTATATCAAATTTTT
AAATGCTCAGTACTATTTGACCATTTAAAAATTTTGTATTCAAACTACCAGTGAAAG
CCCTACCTAGAAGGTATACTCAGTGATAAGTTTTGTAGCTCCAAATCTTCTAATAGT
GAGTGTAACCCCAAAATAAAAGGCTGACAGGTAAGTCGAGAATACTCACTTAATTC
TGGTAAGAAAGCAACCCATTTGTACTTGTATTTACCAGCAATCCTTAAAATGAAGCT
TCCTACTAACTCAATAGCAATAAGACAATAGTGAATGTTTAATGAAAACAGTATTTT
ATAAATACTTTAATAAAAAGGATTGTGATGAAGAACAATCTATTTATATTTGTTATTT
GTTTTTAATTCCAATAAAAATAATTTTTAAAATTACAGAAAAAAGTTATTAAGAACC
ATGCTTTTAAATTTAAAATGATTTTTTAAATTTATTCCTGTCTTTTTCTACAAAGAAA
GCATACATTAAGCAAATACCAAAGGCCAGGTTTACATTTGAAGAAAGTGACATTATT
ATTACTCAAGTCTCTAGGAATACTTAACACATCTCTTGACTGTATATGGATGTTAATA
AATAGCTGACAGTAAAGTTTATCCATATAAAGACTTGCAAATATTCCTCTACCAATG
ACGAGACTTTAAAATATCTATAATAATGTAACACATTTCACTGGTGAAACATGTCTT
GTCATATGCATTATAGAAAGGATAATCAGACTTTCAGTTATATTAATATTTTTAACAT
TTTTGTGCACATAGCTATCTTCAATAAAATTGTTTTAAAAGGTATTATTTTAAGATAC
ACTAAAATGATCAAGGGATTCAAGACTAAACAACTCAATTAGTTGCACCAATAAAA
AACACTTAAAAAAACTGTCAGTGTCCAACCTGTACTTAATAACTCACAGATTTTTAA
AACTTTTCTTTTCAGGAGAATTTTGGTTGGGCCTAGAGAAGATATACTCCATAGTGA
AGCAATCTAATTATGTTTTACGAATTGAGTTGGAAGACTGGAAAGACAACAAACATT
ATATTGAATATTCTTTTTACTTGGGAAATCACGAAACCAACTATACGCTACATCTAGT
TGCGATTACTGGCAATGTCCCCAATGCAATCCCGGAAAACAAAGATTTGGTGTTTTC
TACTTGGGATCACAAAGCAAAAGGACACTTCAACTGTCCAGAGGGTTATTCAGGTAT
CTTTTTCTGATACCAATACTTTATTTTCATATCTTCAAAGTATCTTCCCACATTATTAG
CTATTATCTGCAATGACAACTTTTAAAAATCCGAATCCCAAATAAGCGTTTTCTCTCT
AGACGAAAACCTCTTAACTATAATGAAAGTGTTCATTCTAGTTCAATCAGGTATTTT
ACCTCTAATCTTCCTCAGATTTTCTATTTTTTGGTAGTGTATAGATTATTTATACAGAT
TATTTAAAATTGGGACTTATACAGATTATTTAAAACTGGGATACATGCATCTAAAAC
ACTGTAATATTTATAAGAAAGGAAGATAAACTTACGGGGAAATACAGTAACAGTAA
CTACATACGAGTCTGTACCCATTAAATTGCATATCTATCTCCTTTAGGAGGCTGGTGG
TGGCATGATGAGTGTGGAGAAAACAACCTAAATGGTAAATATAACAAACCAAGAGC
AAAATCTAAGCCAGAGAGGAGAAGAGGATTATCTTGGAAGTCTCAAAATGGAAGGT
TATACTCTATAAAATCAACCAAAATGTTGATCCATCCAACAGATTCAGAAAGCTTTG
AATGAACTGAGGCAAATTTAAAAGGCAATAATTTAAACATTAACCTCATTCCAAGTT
AATGTGGTCTAATAATCTGGTATTAAATCCTTAAGAGAAAGCTTGAGAAATAGATTT
TTTTTATCTTAAAGTCACTGTCTATTTAAGATTAAACATACAATCACATAACCTTAAA
GAATACCGTTTACATTTCTCAATCAAAATTCTTATAATACTATTTGTTTTAAATTTTGT
GATGTGGGAATCAATTTTAGATGGTCACAATCTAGATTATAATCAATAGGTGAACTT
ATTAAATAACTTTTCTAAATAAAAAATTTAGAGACTTTTATTTTAAAAGGCATCATAT
GAGCTAATATCACAACTTTCCCAGTTTAAAAAACTAGTACTCTTGTTAAAACTCTAA
ACTTGACTAAATACAGAGGACTGGTAATTGTACAGTTCTTAAATGTTGTAGTATTAA
TTTCAAAACTAAAAATCGTCAGCACAGAGTATGTGTAAAAATCTGTAATACAAATTT
TTAAACTGATGCTTCATTTTGCTACAAAATAATTTGGAGTAAATGTTTGATATGATTT
ATTTATGAAACCTAATGAAGCAGAATTAAATACTGTATTAAAATAAGTTCGCTGTCT
TTAAACAAATGGAGATGACTACTAAGTCACATTGACTTTAACATGAGGTATCACTAT
ACCTTATTTGTTAAAATATATACTGTATACATTTTATATATTTTAACACTTAATACTAT
GAAAACAAATAATTGTAAAGGAATCTTGTCAGATTACAGTAAGAATGAACATATTTG
TGGCATCGAGTTAAAGTTTATATTTCCCCTAAATATGCTGTGATTCTAATACATTCGT
GTAGGTTTTCAAGTAGAAATAAACCTCGTAACAAGTTACTGAACGTTTAAACAGCCT
GACAAGCATGTATATATGTTTAAAATTCAATAAACAAAGACCCAGTCCCTAAATTAT
AGAAATTTAAATTATTCTTGCATGTTTATCGACATCACAACAGATCCCTAAATCCCTA
AATCCCTAAAGATTAGATACAAATTTTTTACCACAGTATCACTTGTCAGAATTTATTT
TTAAATATGATTTTTTAAAACTGCCAGTAAGAAATTTTAAATTAAACCCATTTGTTAA
AGGATATAGTGCCCAAGTTATATGGTGACCTACCTTTGTCAATACTTAGCATTATGTA
TTTCAAATTATCCAATATACATGTCATATATATTTTTATATGTCACATATATAAAAGA
TATGTATGATCTATGTGAATCCTAAGTAAATATTTTGTTCCAGAAAAGTACAAAATA
ATAAAGGTAAAAATAATCTATAATTTTCAGGACCACAGACTAAGCTGTCGAAATTAA
CGCTGATTTTTTTAGGGCCAGAATACCAAAATGGCTCCTCTCTTCCCCCAAAATTGG
ACAATTTCAAATGCAAAATAATTCATTATTTAATATATGAGTTGCTTCCTCTATTTGG
TTTCCTTAAAAAAAAAAAAAACTCTCATAGGACATGTTTCATTTTGTTCCTTTCAGGA
GTAGTAAATTAGACGTTTTCCCCATATAAAGCTTTTTTCTACCAGAAAGATACTTCTG
GTAGAAGAAGAGAAAGGAGCTCTTTATGGTTCACACGACTGTCTCCTGTCCTAACTA
CTTTGCTTAAAGTGCTCAAATTCCATCACTACTCACAGTTGTCTAATCTAAGTCTAAT
CCCCTTTGATCTCTCAGACTACCTTCCCTTTTATCTCTCTACTACTTAATAATAAGAAT
ATCTTTTTTTCAAACTTGACCTTCATTTTGCTTTCACAATACTATACTCTCCATGGATT
ATCCCTTATCTGAATCCATCTTTATAACCCTATTCCTTTCTCATATTTAGTACTGTGGG
CCAATGGACAACCTTCAATCATCTTTTCTACACTGACCCTCAGACATTCTATCTGCTC
TCACGGACTCCTTTATTTACCATGAATAAAGTTCCAAAATCTACATATTCATCCCAAG
TCTCTTTCCAGTTCCCCTTCTTACATTGCCTATTTGCCATTTCTCCCTTCAATACCCTA
TACTTCACTCAAATTCAACATACCAAAAATAAAAGGCCAGGCACGGTGGCTCACAC
CTGTAATCCCAGGACTTTGGGAGGCTGAGGCAGGTGGATCACCTGAGGTCAGGAGT
CTGACCAGCCTGACCAATATGGTGAAACCCCGTCTCTACCTAAAATACAAAAATTAG
CCAGGCGTGGTGGCATGTGCCTACAGTCCCAGCTACTCAAGAGGCTGAGACAGGAG
AATCGCTTGAACCCAGGAGGCGGAGGTTGCAGTGAGCTGAGATCACACCAATGCAC
TGGGTGACAGAACAAGACTGACTCAAAAAAAAATAAATAACAAATTCCCCAGCCCC
TTACTGCTACTGCTATCCCTTTCTACCCACCTTTCCCTCCTTTATACTCTTTCACACCA
TCTTCCTCACTTCTTTATATCCATTAATATGACCAGCATGTTCCCAGTCACAGAAGCC
TGGAACCCGGAAGACATCTCTGGCTTTTCACTCAACTTTGTAAACTACCTCTTTTGTA
TCATAAGCCACCAAGTTCAATACAATCTTCTCTTGAAACGTCTCTTAATCTTATAAGC
TTTCTTCCCCAAAGACTGTCTTTAACTTCAGTGCTAGATTATATAAGT
AAD34156.1 human angiopoietin-related protein 3
(SEQ ID NO: 2)
MFTIKLLLFIVPLVISSRIDQDNSSFDSLSPEPKSRFAMLDDVKILANGLLQLGHGLKDFV
HKTKGQINDIFQKLNIFDQSFYDLSLQTSEIKEEEKELRRTTYKLQVKNEEVKNMSLELN
SKLESLLEEKILLQQKVKYLEEQLTNLIQNQPETPEHPEVTSLKTFVEKQDNSIKDLLQTV
EDQYKQLNQQHSQIKEIENQLRRTSIQEPTEISLSSKPRAPRTTPFLQLNEIRNVKHDGIPA
ECTTIYNRGEHTSGMYAIRPSNSQVFHVYCDVISGSPWTLIQHRIDGSQNFNETWENYKY
GFGRLDGEFWLGLEKIYSIVKQSNYVLRIELEDWKDNKHYIEYSFYLGNHETNYTLHLV
AITGNVPNAIPENKDLVFSTWDHKAKGHFNCPEGYSGGWWWHDECGENNLNGKYNKP
RAKSKPERRRGLSWKSQNGRLYSIKSTKMLIHPTDSESFE

### Proprotein Convertase Subtilisin-Kexin Type 9 (PCSK9)

The target gene for modification using the systems and methods disclosed herein can be a gene encoding PCSK9. Proprotein convertase subtilisin-kexin type 9 (PCSK9), also known as neural apoptosis-regulated convertase 1 (NARC-I), is a proteinase K-like subtilase identified as the 9th member of the secretory subtilase family. “Proprotein convertase subtilisin/kexin type 9 (PCSK9)” refers to an enzyme encoded by the PCSK9 gene. PCSK9 binds to the receptor for low-density lipoprotein (LDL) particles. In the liver, the LDL receptor removes LDL particles from the blood through the endocytosis pathway. When PCSK9 binds to the LDL receptor, the receptor is channeled towards the lysosomal pathway and broken down by proteolytic enzymes, limiting the number of times that a given LDL receptor is able to uptake LDL particles from the blood. Thus, blocking PCSK9 activity may lead to more LDL receptors being recycled and present on the surface of the liver cells, and will remove more LDL cholesterol from the blood.

Therefore, blocking PCSK9 can lower blood cholesterol levels. PCSK9 orthologs are found across many species. PCSK9 is inactive when first synthesized, a pre-pro enzyme, because a section of the peptide chain blocks its activity; proprotein convertases remove that section to activate the enzyme. Pro-PCSK9 is a secreted, globular, serine protease capable of proteolytic auto-processing of its N-terminal pro-domain into a potent endogenous inhibitor of PCSK9, which blocks its catalytic site. PCSK9's role in cholesterol homeostasis has been exploited medically. Drugs that block PCSK9 can lower the blood level of low-density lipoprotein cholesterol (LDL-C). The first two PCSK9 inhibitors, alirocumab and evolocumab, were approved by the U.S. Food and Drug Administration in 2015 for lowering cholesterol where statins and other drugs were insufficient.

The human gene for PCSK9 localizes to human chromosome Ip33-p34.3. PCSK9 is expressed in cells capable of proliferation and differentiation including, for example, hepatocytes, kidney mesenchymal cells, intestinal ileum, and colon epithelia as well as embryonic brain telencephalon neurons. See, e.g., Seidah et al., 2003 PNAS 100:928-933, which is incorporated herein by reference.

Original synthesis of PCSK9 is in the form of an inactive enzyme precursor, or zymogen, of 72-kDa, which undergoes autocatalytic, intramolecular processing in the endoplasmic reticulum (ER) to activate its functionality. This internal processing event has been reported to occur at the SSVFAQ jSIP motif, and has been reported as a requirement of exit from the ER. “j” indicates cleavage site. See, Benjannet et al., 2004 J. Biol. Chem. 279:48865-48875, and Seidah et al, 2003 PNAS 100:928-933, each of which are incorporated herein by reference. The cleaved protein is then secreted. The cleaved peptide remains associated with the activated and secreted enzyme.

The gene sequence for human PCSK9 is ˜22-kb long with 12 exons encoding a 692 amino acid protein. The protein sequence of human PCSK9 can be found, for example, at Deposit No. NP_777596.2, which sequence is incorporated herein in its entirety. Human, mouse and rat PCSK9 nucleic acid sequences have been deposited; see, e.g., GenBank Accession Nos.: AX127530 (also AX207686), AX207688, and AX207690, respectively, each of which sequence is incorporated herein in its entirety. The gene sequence of Macaca fascicularis can be found publically, for example, NCBI Gene ID: 102142788, which sequence is incorporated herein in their entirety. Macaca fascicularis proprotein convertase subtilisin/kexin type 9 isoform X2 sequence can be found publically, for example, at NCBI Reference Sequence: XP_005543317.1, which sequence is incorporated herein in its entirety.

The translated protein contains a signal peptide in the NH2-terminus, and in cells and tissues an about 74 kDa zymogen (precursor) form of the full-length protein is found in the endoplasmic reticulum. During initial processing in the cell, the about 14 kDa prodomain peptide is autocatalytically cleaved to yield a mature about 60 kDa protein containing the catalytic domain and a C-terminal domain often referred to as the cysteine-histidine rich domain (CHRD). This about 60 kDa form of PCSK9 is secreted from liver cells. The secreted form of PCSK9 appears to be the physiologically active species, although an intracellular functional role of the about 60 kDa form has not been ruled out.

Numerous PCSK9 variants are disclosed and/or claimed in several patent publications including, but not limited to the following: PCT Publication Nos. WO2001031007, WO2001057081, WO2002014358, WO2001098468, WO2002102993, WO2002102994, WO2002046383, WO2002090526, WO2001077137, and WO2001034768; US Publication Nos. US 2004/0009553 and US 2003/0119038, and European Publication Nos. EP 1 440 981, EP 1 067 182, and EP 1 471 152, each of which are incorporated herein by reference.

Several mutant forms of PCSK9 are well characterized, including S 127R, N157K, F216L, R218S, and D374Y, with S 127R, F216L, and D374Y being linked to autosomal dominant hypercholesterolemia (ADH). Benjannet et al. (J. Biol. Chem., 279(47):48865-48875 (2004)) demonstrated that the S 127R and D374Y mutations result in a significant decrease in the level of pro-PCSK9 processed in the ER to form the active secreted zymogen. As a consequence, it is believed that wild-type PCSK9 increases the turnover rate of the LDL receptor causing inhibition of LDL clearance (Maxwell et al, PNAS, 102(6):2069-2074 (2005); Benjannet et al, and Lalanne et al), while PCSK9 autosomal dominant mutations result in increased levels of LDLR, increased clearance of circulating LDL, and a corresponding decrease in plasma cholesterol levels. See, Rashid et al, PNAS, 102(15):5374-5379 (2005); Abifadel et al, 2003 Nature Genetics 34: 154-156; Timms et al, 2004 Hum. Genet. 114:349-353; and Leren, 2004 Clin. Genet. 65:419-422, each of which are incorporated herein by reference.

A later-published study on the S127R mutation of Abifadel et al, reported that patients carrying such a mutation exhibited higher total cholesterol and apoB 100 in the plasma attributed to (1) an overproduction of apoB 100-containing lipoproteins, such as low density lipoprotein (LDL), very low density lipoprotein (VLDL) and intermediate density lipoprotein (IDL), and (2) an associated reduction in clearance or conversion of said lipoproteins. Together, the studies referenced above evidence the fact that PCSK9 plays a role in the regulation of LDL production. Expression or upregulation of PCSK9 is associated with increased plasma levels of LDL cholesterol, and inhibition or the lack of expression of PCSK9 is associated with low LDL cholesterol plasma levels. Significantly, lower levels of LDL cholesterol associated with sequence variations in PCSK9 have conferred protection against coronary heart disease; Cohen et al, 2006 N. Engl. J. Med. 354: 1264-1272.

Lalanne et al. demonstrated that LDL catabolism was impaired and apolipoprotein B-containing lipoprotein synthesis was enhanced in two patients harboring S 127R mutations in PCSK9 (J. Lipid Research, 46: 1312-1319 (2005)). Sun et al. also provided evidence that mutant forms of PCSK9 are also the cause of unusually severe dominant hypercholesterolaemia as a consequence of its effect of increasing apolipoprotein B secretion (Sun et al, Hum. Mol. Genet, 14(9): 1161-1169 (2005)). These results were consistent with earlier results which demonstrated adenovirus-mediated overexpression of PCSK9 in mice results in severe hypercholesteromia due to drastic decreases in the amount of LDL receptor Dubuc et al., Thromb. Vase. Biol., 24: 1454-1459 (2004), in addition to results demonstrating mutant forms of PCSK9 also reduce the level of LDL receptor (Park et al., J. Biol. Chem., 279:50630-50638 (2004). The overexpression of PCSK9 in cell lines, including liver-derived cells, and in livers of mice in vivo, results in a pronounced reduction in LDLR protein levels and LDLR functional activity without changes in LDLR mRNA level (Maxwell et al., Proc. Nat. Amer. Set, 101:7100-7105 (2004); Benjannet S. et al, J. Bio. Chem. 279: 48865-48875 (2004)).

Various therapeutic approaches to the inhibition of PSCK9 have been proposed, including: inhibition of PSCK9 synthesis by gene silencing agents, e.g., RNAi; inhibition of PCSK9 binding to LDLR by monoclonal antibodies, small peptides or adnectins; and inhibition of PCSK9 autocatalytic processing by small molecule inhibitors. These strategies have been described in Hedrick et al., Curr Opin Investig Drugs 2009; 10:938-46; Hooper et al, Expert Opin Biol Ther, 2013; 13:429-35; Rhainds et al, Clin Lipid, 2012; 7:621-40; Seidah et al, Expert Opin Ther Targets 2009; 13:19-28; and Seidah et al, Nat Rev Drug Discov 2012; 11:367-83, each of which are incorporated herein by reference.

In some embodiments, the loss of function mutation induced in PCSK9 e.g., G106R, L253F, A443T, R93C, etc. In some embodiments, the loss-of-function mutation is engineered (i.e., not naturally occurring), e.g., G24D, S47F, R46H, S 153N, H193Y, etc.

PCSK9 variants that can be useful in the present disclosure are loss-of-function variants that may boost LDL receptor-mediated clearance of LDL cholesterol, alone or in combination with other genes involved in the pathway, e.g., APOC3, LDL-R, or Idol. In some embodiments, the PCSK9 loss-of-function variants produced using the methods of the present disclosure express efficiently in a cell. In some embodiments, the PCKS9 loss-of-function variants produced using the methods of the present disclosure is activated and exported to engage the clathrin-coated pits from unmodified cells in a paracrine mechanism, thus competing with the wild-type PCSK9 protein. In some embodiments, the PCSK9 loss-of-function variant comprises mutations in residues in the LDL-R bonding region that make direct contact with the LDL-R protein. In some embodiments, the residues in the LDL-R bonding region that make direct contact with the LDL-R protein are selected from the group consisting of R194, R237, F379, S372, D374, D375, D378, R46, R237, and A443.

As described herein, a loss-of-function PCSK9 variant, may have reduced activity compared to a wild type PCSK9 protein. PCSK9 activity refers to any known biological activity of the PCSK9 protein in the art. For example, in some embodiments, PCSK9 activity refers to its protease activity. In some embodiments, PCSK9 activity refers to its ability to be secreted through the cellular secretory pathway. In some embodiments, PCSK9 activity refers to its ability to act as a protein-binding adaptor in clathrin-coated vesicles. In some embodiments, PCSK9 activity refers to its ability to interact with LDL receptor. In some embodiments, PCSK9 activity refers to its ability to prevent LDL receptor recycling. These examples are not meant to be limiting.

In some embodiments, the activity of a loss-of-function PCSK9 variant may be reduced by at least 20%, at least 30%, at least 40%, at least 50%, at least 60%, at least 70%, at least 80%, at least 90%, at least 99%, or more. In some embodiments, the loss-of-function PCSK9 variant has no more than 50%, no more than 40%, no more than 30%, no more than 20%, no more than 10%, no more than 5%, no more than 1% or less activity compared to a wild type PCSK9 protein. Non-limiting, exemplary assays for determining PCSK9 activity have been described in the art, e.g., in US Patent Application Publication US20120082680, which are incorporated herein by reference.

In some embodiments, cellular PCSK9 activity may be reduced by reducing the level of properly folded and active PCSK9 protein. Introducing destabilizing mutations into the wild type PCSK9 protein may cause misfolding or deactivation of the protein. A PCSK9 variant comprising one or more destabilizing mutations described herein may have reduced activity compared to the wild type PCSK9 protein. For example, the activity of a PCSK9 variant comprising one or more destabilizing mutations described herein may be reduced by at least about 20%, at least about 30%, at least about 40%, at least about 50%, at least about 60%, at least about 70%, at least about 80%, at least about 90%, at least about 95%, at least about 99%, or more.

In some embodiments, the methods and composition disclosed herein reduce or abolish expression of protein encoded by a target gene and/or function thereof. For example, the methods and composition disclosed herein reduces expression and/or function of PCSK9 protein encoded by the PCSK9 gene by at least 3-fold, at least 4-fold, at least 5-fold, at least 6-fold, at least 7-fold, at least 8-fold, at least 9-fold, or at least 10-fold relative to a control. For example, the methods and composition disclosed herein reduces expression and/or function of APOC3 protein encoded by the APOC3 gene by at least 3-fold, at least 4-fold, at least 5-fold, at least 6-fold, at least 7-fold, at least 8-fold, at least 9-fold, or at least 10-fold relative to a control. For example, the methods and composition disclosed herein reduces expression and/or function of ANGPTL3 protein encoded by the ANGPTL3 gene by at least 3-fold, at least 4-fold, at least 5-fold, at least 6-fold, at least 7-fold, at least 8-fold, at least 9-fold, or at least 10-fold relative to a control.

In some embodiments, the gene modification methods and compositions disclosed herein reduces expression of functional PCSK9 protein encoded by the PCSK9 gene in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100%. In some embodiments, the modification reduces expression of functional PCSK9 protein encoded by the PCSK9 gene in the cell by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold. In some embodiments, the modification abolishes expression of functional PCSK9 protein encoded by the PCSK9 gene in the cell.

Some aspects of the present disclosure provide strategies of reducing cellular PCSK9 activity via preventing PCSK9 mRNA maturation and production. In some embodiments, such strategies involve alterations of splicing sites in the PCSK9 gene. Altered splicing site may lead to altered splicing and maturation of the PCSK9 mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an in frame stop codon is encountered by the translating ribosome in the intron. In some embodiments, a start codon is edited and protein translation initiates at the next ATG codon, which may not be in the correct coding frame.

The splicing sites typically comprises an intron donor site, a Lariat branch point, and an intron acceptor site. The mechanism of splicing is familiar to those skilled in the art. As a non-limiting example, the intron donor site has a consensus sequence of GGGTRAGT, and the C bases paired with the G bases in the intron donor site consensus sequence may be targeted by the methods and compositions described herein, thereby altering the intron donor site. The Lariat branch point also has consensus sequences, e.g., YTRAC, wherein Y is a pyrimidine and R is a purine. The C base in the Lariat branch point consensus sequence may be targeted by the nucleobase editors described herein, leading to the skipping of the following exon. The intron acceptor site has a consensus sequence of YNCAGG, wherein Y is a pyrimidine and N is any nucleotide. The C base of the consensus sequence of the intron acceptor site, and the C base paired with the G bases in the consensus sequence of the intron acceptor site may be targeted by the nucleobase editors described herein, thereby altering the intron acceptor site, in turn leading the skipping of an exon. As described herein, gene sequence for human PCSK9 is −22-kb long and contains 12 exons and 11 introns. Each of the exon-intron junction may be altered to disrupt the processing and maturation of the PCSK9 mRNA.

In some embodiments, a splice site disruption generated by a base editor system disclosed herein can result in the inclusion of intronic sequences in messenger RNA (mRNA) encoded by the PCSK9 gene. In some embodiments, the splice site disruption generates a nonsense, frameshift, or an in-frame indel mutation that result in premature stop codons or in insertion/deletion of amino acids that disrupt protein activity. In some embodiments, the splice site disruption generates exclusion of exonic sequences. In some embodiments, the splice site disruption generates exclusion of exonic sequences that results in nonsense, frameshift, or in-frame indel mutations in the PCSK9 transcript. Canonical splice donors comprise the DNA sequence GT on the sense strand, whereas canonical splice acceptors comprise the DNA sequence AG. Alteration of the sequence disrupts normal splicing. Splice donors can be disrupted by adenine base editing of the complementary base in the second position in the antisense strand (GT to GC), and splice acceptors can be disrupted by adenine base editing of the first position in the sense strand (AG to GG).

Further, the present disclosure also contemplates the use of destabilizing mutations to counteract the effect of gain-of-function PCSK9 variant. Gain-of-function PCSK9 variants (e.g., the gain-of-function variants have been described in the art and are found to be associated with hypercholesterolemia (e.g., in Peterson et al., J Lipid Res. 2008 June; 49(6): 1152-1156; Benjannet et al., J Biol Chem. 2012 Sep. 28; 287(40):33745-55; Abifadel et al, Atherosclerosis. 2012 August; 223(2):394-400; and Cameron et al, Hum. Mol. Genet. (1 May 2006) 15(9): 1551-1558, each of which is incorporated herein by reference). Introducing destabilizing mutations into these gain-of-function PCSK9 variants may cause misfolding and deactivation of these gain-of-function variants, thereby counteracting the hyper-activity caused by the gain-of-function mutation. Further, gain-of-function mutations in several other key factors in the LDL-R mediated cholesterol clearance pathway, e.g., LDL-R, APOB, or APOC, have also been described in the art. Thus, making destabilizing mutations in these factors to counteract the deleterious effect of the gain-of-function mutation using the compositions and methods described herein, is also within the scope of the present disclosure. As such, the present disclosure further provides mutations that cause misfolding of PCSK9 protein or structurally destabilization of PCSK9 protein.

The polypeptide and coding nucleic acid sequences of PCSK9 and of other members of the family of human origin and those of a number of animals are publicly available, e.g., from the NCBI website or ENSEMBL website. Examples include, but are not limited to the following sequences, each of which sequences are incorporated herein in their entireties;

Wild Type PCSK9 Gene (NG_009061.1), Homo sapiens proprotein convertase
subtilisin/kexin type 9 (PCSK9), RefSeqGene (LRG_275) on chromosome 1
(SEQ ID NO: 755)
GTCCGATGGGGCTCTGGTGGCGTGATCTGCGCGCCCCAGGCGTCAAGCACCCACACC
CTAGAAGGTTTCCGCAGCGACGTCGAGGCGCTCATGGTTGCAGGCGGGCGCCGCCG
TTCAGTTCAGGGTCTGAGCCTGGAGGAGTGAGCCAGGCAGTGAGACTGGCTCGGGC
GGGCCGGGACGCGTCGTTGCAGCAGCGGCTCCCAGCTCCCAGCCAGGATTCCGCGC
GCCCCTTCACGCGCCCTGCTCCTGAACTTCAGCTCCTGCACAGTCCTCCCCACCGCAA
GGCTCAAGGCGCCGCCGGCGTGGACCGCGCACGGCCTCTAGGTCTCCTCGCCAGGA
CAGCAACCTCTCCCCTGGCCCTCATGGGCACCGTCAGCTCCAGGCGGTCCTGGTGGC
CGCTGCCACTGCTGCTGCTGCTGCTGCTGCTCCTGGGTCCCGCGGGCGCCCGTGCGC
AGGAGGACGAGGACGGCGACTACGAGGAGCTGGTGCTAGCCTTGCGTTCCGAGGAG
GACGGCCTGGCCGAAGCACCCGAGCACGGAACCACAGCCACCTTCCACCGCTGCGC
CAAGGTGCGGGTGTAGGGATGGGAGGCCGGGGCGAACCCGCAGCCGGGACGGTGC
GGTGCTGTTTCCTCTCGGGCCTCAGTTTCCCCCCATGTAAGAGAGGAAGTGGAGTGC
AGGTCGCCGAGGGCTCTTCGCTTGGCACGATCTTGGGGACTGCAGGCAAGGCGGCG
GGGGAGGACGGGTAGTGGGGAGCACGGTGGAGAGCGGGGACGGCCGGCTCTTTGG
GGACTTGCTGGGGCGTGCGGCTGCGCTATTCAGTGGGAAGGTTCGCGGGGTTGGGA
GACCCGGAGGCCGAGGAAGGGCGAGCAGAGCACTGCCAGGATATCCTGCCCAGATT
TCCCAGTTTCTGCCTCGCCGCGGCACAGGTGGGTGAAGGAGTGAATGCCTGGAACGT
ACTGGGAACTGCACCAGGCACAGAGAAAGCGGGCTTGCCATTATAGTGGGTTCCGA
TTTGGTTTGGAAAACATGGGCAGCGGAGGGTGGAGGGCCTGGAGAGAAGGCCCTAC
CCGAGACAGGGGGGGGGTGGGAAGGACGGCAGATGCTGGGAGCACGAGGCAATTT
CTTTATGACACAGAACTCATGCTCTAGTATTCCATCTGTTTCAGCCGAAGAAAAGAA
CCAGCTGAAGGGGCAGGGGAGAAGGGGCGGAGGTATTCTCGAGGCCCATTGGCGTC
CTTTAGGACTCAGGCAGGGAAGGGCCCTTGGTGCTCTGGAGCCGGAGGTGGTGCGC
CTGGTACTGGGACCCCGGAGCTGAGCCCGGCGCCTCAGCCCACCTGGCTGTCTGCCG
ACCGTGTGCGGGGCGAGTTTGCTCAACAACTCTGCCAGCTTCTGGCCCTCAGGCTGT
GGGAAGCTTCTTCCCGGGGCGAGACCACTAGCTTTTTCTAAGTATTACCAGCCCAGG
ACTTGGCTGAGGTTCTGTGTCCCCCAGCTTGGAGTCAGATGTGGGGTTGAATCTTGG
CTTCCTCTCACTAGCTGTGGTGCTTGACAAGTCACTTATCCTTGAGCCTCCATTGCCT
AATCTTTAAAAGGGAGGTGACAATCGTCCCTACGGCTCAGTGGCAGCAGATGGGGA
GATGAAGGGAAAGTTCTGTTGACCATGAGTGAACTTACAATGCAAGCCCCGGGGGG
ATCACTTGCAGTTTTGTCCCTGTCTGCAGTGTGACCTGTTGGTGACATTGTCTTTGCT
CCAAACCACAGCTCCTGGGGCAGAGGGGAAAATTCTGCCACTCACAGCTGCCTGCC
CACGCTTCTGTCTGAGTGTGCTGGGTGGCAGGATGGCAAGTCCTTACTCAGCTCAGT
ATAGCCCTCTTCCTTGTTCCCTGAGCCTTTGACTTTCTCGAGGGATGTTGTGGGGTTG
TGGCCAGGATAAGAAAGGGCATTTCAAGTTACCACTGCTCCAAAACAACTGTTCTGG
AAATAGTGAGTACCCCATCCTGAGAGGTGAGTAAGCAGAGGCTGTATGACCACCTG
AACCAAGCCCTTGAGGATGTTTCTTCTCTGGTGGAAGTTTGGAACAGGAGCCTCCTC
AAGTTCATTTATTCATTCATTCAATGGTTATTTTGTGGGAATCGAATTTAGAATGAAA
ATATTTTTTGGCAAGCAGAAAATAATTTTTAGACCAATCCTTTTCTTTTAGTCATGAG
AAACTGAGGCCCAGAGAGAGGAGGTCACCCCAGGTGCATTAGAACTGGGTTTCCAG
AACTGACACTCCACTGCACAGAGTACTCTCCCAATTCATTCAATTTTTATTTAGCGGA
AGGCATTTTCAGATGGGTCTTTGAAGCATTAGTAGGAGTTCAGCGATGATGGTGTCA
TGAGAATTTTATTCTAGGATTAGGAGGTACCATGAACAAAGATACAGAGCTGGGAA
AACCAGAGGTGGAAGATAAGGAGCACATGTCCACAGTTCTTTTTCTTTTTTTTTTGAG
ATGGAGTTTCGCTCTTGTTGCCCAGGCTGGAGTGCAATGGTGCAGTCTCAGCTCACT
GCAACATCTGTCTCCCGGGTTCAAGTGGTTCTCCTGCCTCAGCCTCCCAAGAAGCTG
GGATTACAGGTACCTGCCACCACGCCCGGCTAATTTTTGTATTTTTAGTAGAGAAGG
GGTTTCACCACGTTGGCCAGGCTAGTCGCAAACTCCTGACCTCCTCAGTGGATCCGA
GGAGGTGATCCTCCCGCCTCAGCCTCCCAAAGTGCTCGAATTACAGGTGTGAGCCAC
CACGCCTGGCCTCCACAGTTCTTTATCCACCGTCTGAAATGTAAAATGTTACGAAAA
CCAAAAGTTTTTTTTGTGATTTATTTGATGGTAGCACCTGACGTGAACTGACATGAGA
TTATTTTTAATTTAGTTGTGTGAATATGCATATTCATATATTTTGCTGCATAGATTACA
GTATGCAGCTCCAGATTCTTCCAAGCAGACTCTGATTGCCCATTACTGCCTTTCTAAA
ATCCAAACAAGTTCTGAGGTTCAAAACCGTTTTGGCCCTAAGGCTTTGGGTAAAGGG
GGTGGACTCTGTTCTACTCTGACTGGAGTCCAAGATGCATATATACAGAGATATGGG
TGATGGGGCTGCAAGGTAGGTTGAGGTAGGGGCCAAGGAGGAGCATGGAGTTTGGA
CTTGATTCATGAGGCTGTGGGGAGCCAGTGAAGGTTCTTAAGCAGGTATGTCTGCCT
GAGAGCAGTTGGAGCAGACAAGAGCTAAAAACCAAACAAATCACCATAGATAGTG
GCTGCTATAATTTGTTTGTCCCCTCCAAATCTCATGTGGAAATTTGGTCCTCAGTGTT
GGAAGTGGGGCCTAATGGGAGGTGTTTGGGTCATGGGGGAGGAACCCCTGTGAAAG
GCTTGGTGCCGTCCTTGTGATAATGAGTAAGTTCTCCCGCTATGATTTCCCTTGAAGG
CTGATTATTAAAAAGAGCTTGGCACCTCCCTCTCTTCTCTCTTGCTTCTTCTCTTGCCA
TGTGATTGATCTCTGCACATGTAGGCTCCCCTTCACCTTCTGCCATCAGTGAAAGCAG
CTTAAGGCCCTCACCAGAAGCAGATGCTGGTGCCATGCTTCCTGGAGAGCTTGCAGA
ATCATGAGCTGAATAAATCCCTTTTCCTTGTAAATTACTCACCTTCAGGTATTCCTTT
ATATAGCAACACAAAAGGACTAAGACAGTGGCCTTGACTTTTCTCTCTCTTTAAGAA
GTGTTGCCTTTGCTCACTTAGTCATCCCTTCTGCCTGCATTTGTAGAGCATCTGGATG
GGAGATTTATATAACCGTCACTCTTGACTTTCCCAGCAGGCCTATGTCATAGGTACT
GTGGTCTCTACAATACAGCAGAGGTATCTGAGGCTCCGAGAGGTTGAGTGACTTGCT
CATGGCTGCACAACCAGTAAATATTGGAGCTGGAATTCAGGTCCACGGTTTCCTGGC
TCCAAAGCCCATGATTTTTTCCCTCAATTTATTCTGACTGGGGCATGGGGGAGGGGG
TGGCCTTTGGGCAGGGCCACCAGGAGCGACCAGGCCCGTAGAGAGCTGGGTGCAGG
TACAGAGGAAAACCTGTTGTCGAGTGTGGCCCGTAGTTCCCATTTTTGCCTGAATGG
CACATTTGAAAGTGTTATATAACCATGTGAATAATAATAGTTGGCCTATATGAGTTC
TTTAATTTGCTTTTTGGTCCGCATTTGGTAACTTCTTTATCATCTACTATACTCTGTTG
TGTCTCTTTTGTTGTAATTTGTAAGTAGGGGTGAGATAAAGTACACCTAGGGTTTGCT
GGGTTTCTTCCATGTCATCATGTTCCTCCTTGCATGGGGCCAGGATCCGTGGAGGTTG
CCTGGCACCTACGTGGTGGTGCTGAAGGAGGAGACCCACCTCTCGCAGTCAGAGCG
CACTGCCCGCCGCCTGCAGGCCCAGGCTGCCCGCCGGGGATACCTCACCAAGATCCT
GCATGTCTTCCATGGCCTTCTTCCTGGCTTCCTGGTGAAGATGAGTGGCGACCTGCTG
GAGCTGGTGAGCCACCCTTTTTGGGAATGGCACTTCCTGATAGGGCTGGGCCACTGC
ATATACACTGGGGACTGTGCTTAGTAGGCCCATTGCTGAAAATCAGAAGGGGACAG
CAAGTATGTATTGAGCACTTATCGGGTACCAAGCACAGTAACTACTGGCTTTCTGTA
TAGAATTCCCTTTAAGCCTGGCCATGCCCCAGTGGTACGTCTATCTTCATTTGAAAGA
CGAGGAGACTGAAGTTCAGAGGGGACCACACAGACAGCTAGGGGTAGAGCCTGGAT
CAAACCCATTGGTCTGCCTGCCAGCCATTCTTGTGCCAATGCATCTGCTGCCTACGG
AAACCTGTAGGGACAAGGCCCTGGGATGTTCAGTGGAGCCTGAGTCATTTTATAAAA
AAGCATGACTCTAGGGTCCAAAATTCCTTTGAAGCTGTTGCTATCCAGAGTGAAGTC
CCTTCTTTAGGACAGGGTGGCCCTCCTCCCTCCTGGATGTCACATCTTCGGTGGAGG
GGCAGAAAGGGGACTGGGTATTCTCCTCACCCTGGCCCTAGTGCTTCAAATCTTAAA
AAAACGTTTTTATTTGTGCTTCTGCACCACCTTCTAGCCCACCTCGTTTCCTGGCCTCT
AACTTGATGAGAGCGTGTGTCATTTTCACACTGATTCTCCACATGGCAGGCGGTGCT
TCTTAGCCTCCTGCAGACAGTGAGGCCCCACGGTCTTGTCCAAGGTCACACAGCGTG
TAATGGGCAGGGTCAGAGTCTGGAGTCTGGACCTGGGTCTCCTAGCTGCACTGCACT
GCTGCCCCATGGGTTAATCAGCTCAGCATACCGTGGCTGAACAGCTACCTCATACCA
AGGCCTGTGGCGCCATGACAGGGATTGACAGGGTCCCTGCCTTGGAAACCCGTAGTC
TAAGTAGAGGAGACTGACAAGTCAATGCCTTCCATCAGTCTGCTCAACACACGTTTA
CCAAGTGCCTACTGTGTGCTGCAGAGGCGAAGATGACACAGCTCAGGCCTTTCCCTT
GAGCTTACAGTTCAGGAGGAGAGACTGACCAGTGACTGCCAGTACAGTTGACTATG
GGACAATGTGCTCAGCCTTGGGGAGAGACGAAGAAGGTACCCGTATAGCACCAGAT
GACAGGCACGAGCCCCACAGGCCAGGGCAGCTGCTCAGAGGAGAGTAGGCCAAGC
AGAAGGCAAACAGAAGGCTGCAGGCATTTGCCATCGAGAGCTGGACTTCAAACTGG
GCATCATACCAGCCTGGGTTCGAGTCCTGCCCAGCCCCTTATTGGCTGTCTAACCCTG
AGCAAATCCCTTCACCTCTCTGAGCCTCATTCCTCTATCTGTAAACCAGTTATAATAA
TTGGAACATTCATTTAAGGACTAAATGAGGTCGTGAAGCATTCAGCAGATGCTAGGT
ACGGAAACTCGCTGAAGTGGGGGCAGGTTAAGAAGCCTCTGGGGATACGAAGGCAT
CCAGGGACTAGTTGTGGCAGGAGGCTGTTACCACTTAGGTCTGAAGGGTAAGGAGA
GGGAATAGCTTTCCCTCTGCCCAGTTGGAGCCGGTGGCATGGAGGAGAGGCTGCCTG
TGGGGAATCACCCGAGGGTTCACCGCTGCCATGCGCAGGGAGTCAGGAGGTAGGGA
GGGAGTGGGGCAGATGCACACCATTTTTTTTTTTTTTTGAGACTCTGTTGCCCAGACT
GGAGTGCAGTGGTGCCATATCTGCACCTCTGCCTCCCGGGTTCAAGCTCACTGCAAC
CTCTGCCTCCCGGGTTCAAGCGATTCTCCTGCCTCAGCCTCCCGAGTAGCTGGGACT
ACAGGTGTGTGCCACCATGCCTGGCTAATTTTTGTATTTTTAATAGAGATGGGGTTTC
ACCATGTTGGCCAGGCTGGTCTCGAACTCTCGACCTCAGGTGATCCCCCACCTCGGC
CTCCCAAAGTGCTGGGATTACAGGCGTGAGTCACCGCTCCCAGCTGCTGATGCACTC
TTGTCCTTCTAACTCCTGCTAGTGCCTCCCATTGGCTGAGCCCAACTGGAAGCTTTGC
AAGGGAGCTGGTGCTGCAGTTTGCACTGAGCAGGCTGGAGAAGGCTGGAGAATAGA
CTAGGGGACAAACCGAATTGCCAGTGCTGTTATGTCATGATTTAGGCATGGAGTCCA
GGGCCTGAGCTTCACTCCATGTCCATCCTGCCCAGAGCCTTGGCACAGCCTGGCTCC
CAGACAAGATGTCAAGTTCAGAATCCTTCCTAAAAGGAATCCTCTATGCCAGACCGT
GTTGCAGGGATATGGGAGTGCTGGGCTCCCAGCCTGATCAAGGAGCGAGAAAACTC
AGGCTCCTAGTCTGTCCTCCGGGGCACTAGCAGGGACAAGGTGGGAGGCTGCTGGG
CTGGGATGTGGGGACAGGTTTGATCAGGTAAGGCCAGGCTGTGGCTGTGTTTGCTGC
TGTCCAAATGGCTTAAGCAGAGTCCCCCGGCCTCTCTGGCTTCTGCAGGCCTTGAAG
TTGCCCCATGTCGACTACATCGAGGAGGACTCCTCTGTCTTTGCCCAGAGCATCCCG
TGGAACCTGGAGCGGATTACCCCTCCACGGTACCGGGCGGATGAATACCAGCCCCC
CGGTAAGACCCCCATCTGTGCCCTGCCCCACCCCATCTGAGCTGAATCCATTTGCTCT
GCCCTGGCCTGGCCTCCCTGCTGGTGGTTTCCACTTCTCGGGGGGCTTTGGGACTCAG
CACCTCCACTGACCCCTTTTTTTCTGTCCCATCCCCATCCCCTGCAGCCCCCACTGCC
TGCCTTCCTGTTGCCCCACAAATGCAAAAGTCTTGCCTTAAATGATCCTCTTTTCCTT
CTTTTCTCTTGTTTTCCTTTTCTCACCATTTGGAATGGCCCAGCAGGCTGCACTTACCT
TGGAAGGAGGGTTCATCTGATGGTGACTCTACCTAGGGCCCCCAGGCCTCTATAACT
CCCAGTGCCCTGCAGACTGGACCAGATCCTTTAATGGGATAGACACAACCCTGTCTG
GGATGCCTCTGCCTACCTTCCTGTTTTGCTGCTCCACCTGCCTCCAGCTCCGTTTGGC
TTCCTGGGGCTCCCTGCCTGGGCCACTTTGTGTCTTCCCTCTAGGCCTTTCTTTCCACT
GTTCCCTCTGCCTGGTGTGGCCTGGCTATGGAAGGGAGGGAGGAGGAGCGGCCATG
GAAAACGGTCTGCATTCTAGCAGGGACTTGCAGGTGGCAATTCAGTCGGGGAAGAC
TCTAGATGCACCTGGCCTGAGGAGAGAATGAAGGGTTCTAGTTGGACTGTGTTAAGT
TTGAGGTGCCCATGGTGTGAGGTCTGGAGCTCAGCGCAGAGATGATGCAATGTGGT
GGGTCCATGCAACATGGTGCCAGGACGCAGAGCTTGGGGTGAACTCAGCTTTCACCC
CTTACCGGTTCTCGTGGGATCTTGGGAAGCCACTTTCTTCTATGAGCTTTGTCGTTCT
TGTCTGTAAAATGGGCACATAACCCTGTCCCTGTCCTTCTCACAGGTTGCTGTGAGA
CTCCAATGAGTTGAAGGATGTGCAGATGCTTTTGGAAGTGAAAAGTTGGGGGGCTAC
TGTGTGACTTTGCATACACCCAAACTGTGTGACCTTGCATATGTCTGAGTTGCTGCCA
TTGCAACAGATCAGAGCTGGTGGGCTGGGTGTGGAGAAAGGGTTTGTGTGGGGGAC
ATCCTCTGGCAAGGGTGGCAGCAGCAGAAGTGAGGGGCCTGGTCGGTCATGTGTGC
TGACCCGGCCTGGGCAGCCTGTGGCCAGGGAGAGGACAGCTCCTCTGTAGGAAGAG
CCTGTTCCTTTCCAACCAGGTGAGACCTCTTCAGTGGAGCCCTGGAGCCCCCTGTACT
CCACATCAGTGCCTCAGGGACCTCCCGGAGCAGGCTAATATCAGAGACCAAGAGGG
ACACTGGCAGAGGATCACAGAGACCCCAGTCCAGGCAGGGACTGAGAAGATCTTGC
CCCCTAAGTTAGTTTCCTAGCACTGCTGTGACAAATTACCACCCCCTCGGTTGGAAC
AAGTTGATTCTCTGCAGTCCTGGAGGCCAGAAGCCTGAATCAGTGTCGGCAGGACCA
CTTTCTCCCGGGGGGCTCCAGGGAGAAGCTTCTCTTGCCTCTTCCGTGTCCCAACAGC
GGCAGCACACCAATCCCAGCCTCTGTCTTCACACAGCCTTCTCTGTGTCTCTCTCCTC
TTCATTGTCTCATAAGGACACTTGTCATTGGATTTAGGGCCCACTGGATCCTCCAGG
ATGATCTCATGTGGGGAACCTTAACCACATCTGCAAGGACCCTTTTTCCAAATAAGG
TCACAGCCACAGGTTGTGGGGGTTAGGATGTGAGTGTATCTCTTTGGCAGCCACTGT
TCCCTCCTCTCCCTTGGGCCAGAAGCAGACGTGGGGCCCTTTCTTCCCCATAGGATG
CCCATGGATTGCCCCCCTTCCCGCTTCCCCCGAGTGTCTGTGGGAGGTGGCAGGAAT
GGCAGGCAGGGGTGTGGAACCCCTTCTGGAGTCATATCAAGGGCTTGGCTGGAGGA
AGTCCTCCTGGAGCTGTTGGGCTGGCATGGGGCAGGCTGGCTGGGCCCAGCAGCAG
CTTCTTCATTCATGGGGAGGCCACAAGCATGGGCCCTAGAGCTGGCTGCCGCCCTCA
AACCCAGACCCTGCACTCTTAACTGTGTGACCTTGCATACGTCACTCACCCTCTCTGA
TCTTCAGGTTCCTCTGCAAAAGGGAGGTAATGATAACCCTCACTCTGGGGGGCTGTT
TGGAGGGTTAAATCAGTTATTGCTGTAGCATGCATTTCTCTGTCAGGTATTGAGTGA
GGTGCTGTGATTTTAGCCCTGCATTTTTCTTTTCTTACCATTCAATAATAACGTTTTGA
GCACCCACTGTGCGCCAGGCACCATATTAGGTGCTGGGGATACAAATGTGAATGAA
ATGAATGTGGTCTCTTCCCCCAACAGTGTATCCAGAAGATTAATCCATTCCTTAAAC
AAATGCTACTTGACACAGATTAGTTCTGGATAGGCTGAGAGCTCTGAAGGAGTGCA
GGCAGCTGCGAGCCTGTGTATCCAGCAGAAGGATCAGGAAAGGATTCCTGGAGGAA
GCGCTGTTCTAGCCAAGACCTACGGGGGCATTATTAACCAGGCAAAGGGGACGGTG
TCCAAGCAGTGGAATGAACGTGGATTGAAGCTGTGAGGCAGGAGGGAGTGTGGCCT
GTGCAGAAGGGACCGAGGCTGGTGAGACCAGGAGGGCCTGGGTGGCCTCCAGGTCA
GATGTGAAAGGAAGAACTTGGCCACAGTCTGAGCTTCTCAGGCGTATGGCAGGGCT
GCCTGGTGAGAGGGAATGAGCTCCCTGCTCTGGAGGTATGCAAGCAGGACTGGGCT
CTCACCTGCCAGAGGCCACAGAGCTTTCCAGAGGCTGGAAGAGGCCACTCCAAGGC
CTCTTTGCCCCTGAGAGTGGTGGCTCTTCTTGAGGCCACCTTGCCACGCTGTCACAGG
GAACTAGCAGCCCCTGCCTCACCCGGGGGTTTGGAAGATAGAGGGAGGCCTAGGAA
GGGCCCTGTGTCTCATCCGAGCTGGGCCCCTTTCCAGCCTCTCACTGGAAGGAAGCC
CAAGGATGTTCCTGTGGGGGCTTTTACCAGGCCCACCTGCCCTCTGCTGGCCATGCTT
GCAGCCTCCTGACCCTGTCCCAGCAGGACAGTGGGCTGGTGTGAGCGGGCAGGAAC
CGCCTGCACTTAGAAGGTGTGGGGCTGCCTCCCCGAGCTTCCATCTGCCGCTGGGGC
CACACCCCAGGCCCAGGGATGGGACCCCACAGTGGTCACATCATCTTGCAGCAGAA
CCCAGGTACAGCTCCTGGAGCAGATGGTGGTCCCAAGCACGGGTGGGACCAGAAAG
GACTCTCACCTGGGCTAACTCAGCTGCAGCCTCAGTTCCCTCCTCACACACGACGAG
GAACATGGACTGGAAGCCTGCCCAGCAGGCCTTCTGCTCGATGTGCGTTGTGTGGCT
TACGTCCAGGGAGGGAAGCAGCCTCTGTGCTGTCTTCTAGATAAGCCTGTATTCCCC
GGGCTGTCTGCCAATGTATCCAGTTGTCCCGTCAGCCTGGAAGCTCTGAGGGAAAAC
CTTGGGCTGCTTCCTGAGCACCTGTATCCCCTGCAGCCAGCCCGGGGCCTCTGCTAG
GAGCAGACTGAGCATGGCTTATGGGCCTGGCACCATCTGGCCTCTGCCCACCTTGCT
GGCCTTGTCTTGTGTCTGCCCCTTCGACATTCCATAGCCCAGCTCAATATCTAGTGGT
TCCTCTAGGGTGGCGAGCACTGTTTGGTCTCCAGATGTCTTCAGGTCGGAGCTCACA
GCGCTCTCAGCCACCCCTTCCCAGTGTAGCACCGGGCACATGGTAGATGCCTATTGA
TGAGTGAAAGCTCCTAACACACTCAGAGAGCAAGGACTCCGCCTCATCCCACAGCCT
GGGAGGAGAGGCAGACTGCCAAGGACCTGCTCAGCATGCTACAGAAGAAACCAAA
GTGCCCACGGGACTGATCAGTGGAGCTTCCTGCCGAGACTGGAGGCCTTAGGGCAG
GGTAGACAGTGTGTGTGCAGGCTGGGGACTCACAGTTCGGACTGTGCCCAGACCTAC
TAGCATAGTGGGTGGGTGGGAGGATGCGGGACTGGGGGCCGACCTTGCCTGAAATT
CATGTGGGATCTCAGAGCAGCCACTGAATTGCTCTGTAGGGGGCTAAATAGTGGCCC
CCACAGATACACACACCCAGACAGAGCCTGTGAGCCAGACCTTATTTGGAGAAAAG
GTCTTTGTAGATGTAATTAAGCATCTCAAGATGGCATCATCTGGATTATGCGGTGGG
CTGTAAGTCCTGTGATGTGTCTTTATGAGAGAAAGGCAGAGGGAGATTTGACACACA
CAGGAGGGGCCACGTGGAGACAGAGGTGGAGATTGGAGAAATGTGGCCACAAGCC
AGGGAACACCAGCAGCCACCAGAAGCCGGAAGACGTGAGGCAGGGTTCTTCCCAGA
GCCTTCGCTGCTGAGTCTGGGAATTTGTGACCGAAGCCATAAGAAGTGGGTACACGC
CCTGAGCCTCCCACACTTGCTCACCTGTCCTGAGATGAGAATCTCTACTCTGCAGCAT
ATTTGGAGGATCACTGCGGGGGCCACAGAGGTGCTGTTCAGATGGCACTTCAGAAG
ACTCAGGAGACCCTGGGGCAGGAGCAGTTTGACTGACAGCCCAGAGGGCTGCCCTC
TGATTCCACCTGAGGCCCTGCTTTTCCTGGCTGCAGGGGTTCCAGGGCCAGGCCATT
TCCGCTGGCGCAGGACTCTGCTAGCAGCAACCTGCCTGAAGTCTTCCTTTGGCCTGG
CTGAGAGTTTCTGAGACCTGCGCTGGAGCGGAGGTGCTTCCTTCCTTGCTTCCTTTCT
TCCTCTCTCCCTTCTCCATCCAGCAGGCTGGACCTGCCTGGCATCTGTGAGCTCTCCC
TACTTTCTCCTATACCCTAACCTTTGTCCTGCATGGGCGACTCCCCCAGTGAGTCTCT
TGCAGCTTTTACCCCAGTGCCTGCTTCTTGGAGAATCCAAACTGATCCAGTTAGGGA
TGATAAAGTGTAGGGTAGGCGCTCGGTGACTGTTTTCTCTGAGGTTGTGACTCGTGT
GAGGCAGAAGCAGTCCCCGTGAGCCCTCCTGGTATCTTGTGGAGTGGAGAACGCTTG
GACCTGGAGCCAGGAGGCCCAGACATACATCCTGTCCGAGCTGCAGCTTCCTGTCTC
TAAAATGAGCCGGCCAGCGCAGGTGGCCAGACATCACTGTTATTCTCCTTTGAGTCT
TTAAATCTTGTTGTCTTTCTTGCAGACTCGGTGAGCTGTGAAAGGCTATAATAGGGG
CTTTATTTTACACTTTGATACTATTTTTTGAACATTCATATTATTGTTAGATATTGATA
TTCATATGAAGGAGCAGGATGACTTGGGTCCTTCTTGGCAGTAGCATTGCCAGCTGA
TGGCCTTGGACAGTTACCTGCCCTCTCTAGGCCTCCCTTTCCTTGTCTATGAAATACA
TTATAGAATAGGATGTAGTGTGTGAGGATTTTTTGGAGGTTAAACGAGTGAATATAT
TTAAGGCGCTTTCACCAGTGCCTGGGATGTGCTCTGTAGTTTCTGTGTGTTAACTATA
AGGTTGACTTTATGCTCATTCCCTCCTCTCCCACAAATGTCGCCTTGGAAAGACGGA
GGCAGCCTGGTGGAGGTGTATCTCCTAGACACCAGCATACAGAGTGACCACCGGGA
AATCGAGGGCAGGGTCATGGTCACCGACTTCGAGAATGTGCCCGAGGAGGACGGGA
CCCGCTTCCACAGACAGGTAAGCACGGCCGTCTGATGGGAGGGCTGCCTCTGCCCAT
ATCCCCATCCTGGAGGTGGGTGGGGACTGCCACCCCAGAGCGTTGCAGCTGTACTCC
TGGGTTGCACCCCCCCCAGCTGTCACTGTCCCCTCCCTGCCATCAGTTGTGGGAAGG
GCGTTCATCCATCCAGCCACCTGCTGATTTGTTATAGGGTGGAGGGGGGGTCTTTCT
CATGTGGTCCTTGTGTTCGTCGAGCAGGCCAGCAAGTGTGACAGTCATGGCACCCAC
CTGGCAGGGGTGGTCAGCGGCCGGGATGCCGGCGTGGCCAAGGGTGCCAGCATGCG
CAGCCTGCGCGTGCTCAACTGCCAAGGGAAGGGCACGGTTAGCGGCACCCTCATAG
GTAAGTGATGGCCCCAGACGCTGGTCTCTCTCCATCTGGACCTGGCCTGGGAGGTGG
CTTGGGCTGGGCCCAGGGAGAGCTAATGTCTCCTAACCAAGAATGCTGTGGCAGCCT
CTGCCGCAGAGCCAGAGAACCAGAGTGCCAAGGCTGGCAGGGTTCCCAGTGGCCAC
GAGTGCAGATGAAGAAACCCAGGCCCCAAGAGGGTCATGCAGGTAGCCCAGGGAGT
TCAGCCTTGACCCTGGGTCAATGACCTTTCCACAGTTCCACACTGCTCCCCTTTTAAA
ATCCGGTGATGTCTTTATGTCTTTTGTTATGTTATCTTCAATGTGGAGGGACTCGAGG
TGATCTAAGCAAACTTTTTCTATCTTCTGCTTGCATACCTCTGAGACCAGGGGACTCA
CTCACTTGCATGACTGGGCCCTGCAGGTCACACTGGCCAGGCAGATGTGGTGGAGG
AACTGGCAGAGGACTTTTTCTAGACTGTGACTACATTTAGTCCACCCAGCGGCCCCC
CTATGAAGTCCAGTTGAGAACTAGGACTCTGGGGGCCGGTGGACAGAGAAGAGGGA
GGGTTCTCTCCCTTACTGACTTCCTTCTGTGGCCAGACATTGAGCAAGGCCTCTGTAC
AGCATGTCCTGGGGCTGGCCTTGCCGTAGCTGCTAAATAGTTGACGAAACCAGTCCA
GAGAGGGGAGGTGACTGCCAGGGTCGCACAGCTCAAGCTGGGGAACTCGCTGGGAA
AACTGTCAGCTCTGGGCAGCAGCTTGACTTCCACTGTAAGCCCCAGCCCCCAGGGTC
AAACACTGGCTCTGGTGCTGGCAGAGGCAGCCCACTAGCCTGTTTCAAAGGCTGAG
AAGGCCCAGGAGTCTGCCCTGTGCTCCACCAGTTCTGCCCTGAGACTTTCCTACAGA
GTACAGGTTTTGATGTTCAGTTTTAAAGGCAAGAATCAATAACCTTCTGCCCCATCA
GGTGACCCCTTGTGCCTGTCCCACCCCTTTATTGACTGACCTCGGCTCAGTCAGGTCA
GTTCCTGAAGGTCAGTGTGTGGAGGGGAGGCTGTTCTTTCCCAGAAAGGCCTTCCCC
AGGCCTGGTGCTCTGGCCTCTGGAGGACTTCCTGGAGAAGTCCCTTCTTTGGGGTCC
CAGTCAGTGTATGGGAAGCCCTTATTGCATGACCTGGCACGGGGCAGGGGCTCAAC
AGTCACTATTGCCTTCCTTGCCACTGCCATTTCCTCCTCTGTAAGCAGGTGATTGTGT
GTCCAGTCTGAGCACAGAGATAAGCACACAGCAGGTGCTTAATAACTAGCAGCTGT
AGGCTGGGCGCGGTGGCTCATGCCTGTAATCCCAGCACTTTGGGAGGCCGAGGTGG
GCAGATCACCTGAGGTCAGGAGTTCGAGACCAGCCTGTTCAACATGGTGAAACCCC
GTCTCTACTAAAAATACAAAAATTAGCCAGGCATGGTGGTGGGTGTCTGTATCCCAG
CTACTTGGGAGGCTAAGGCAGGAGAATCGCTTGAACCCAGGAGGTGGAGGTTGCAG
TGAGCTGAGATCGTGCCACTGCAATCCAGCCTGAGTGATAGAGCGAGATTCCATCTC
AAAAATAAATAAGTAAATAACTAGCAGCTGTAAATGTGGCTGTTGTTCTTCACCTCC
ACACTCAGTGCCACTCCACTCCCTCCCTCCGTGGTGTGAGGGGCCTCACTAGCTGTCT
CCTAGGAGGAGCATGGCTGTGAGATTCCAGCTCCATCCTTGGCCACGGCTCCTGGAG
ACATCTTAGAGGCCAGGATCCAGAAGGCTCCCACACCTCATTTGACAGGGGAGAAG
CTGTCAGTTCCAGGTCCCCTTGCACATCAGGGCCAGAGCTGCGTTAGGCCTCCAGTC
TCCAGGCCACTGGGCCAGAGCTCACAGGCTGGCAGAGGGTTAGAACTGTTACTGGT
GGCTGGGTGCAGTGGCTCACGCCTGTAATCTTAGCACTTTGGGAGGGCAAGGCGGG
AGGATCATGAGGTCAGGACATCGAGACCATCCTTGCTAACACGGTGAAGCCCCGTCT
CTACTAAAACTACAAAAAATTAGCCGGGCGTGGTGGCAGGCGCCTGTAGTCCCAGC
TACTCAGGAGGCTGAGGCAGGAGAATGGCGTGAACCCGGGAGGCGGAGCTTGCAGT
GAGCCGAGATTGCGCCACTGCACTCCAGCCTGGGCAATAGAGCGAGACTCCGTCTG
GAAAGAAAAAAAAAAAAAAGAGCTGTTACTGTTGACAGTAGCATGAGGTAGACCAT
GGCCTGCACCAAAATGGGGGAGTGGAGTGCCACTGAGGCCAGAAGGAACCACACCC
TCAAGGGTGGGGAGTTATGGTATGGGGGGTCCTAGGCATGGAGTCTTTTAATTCTTT
AGACAATCCTGGGAGCAACTGTCCCTGTTTCACAGAGGGCGGGGCCACACAGCTGG
TGAGTGGGCAGCCAAGACTCTGTTCAAGTTTGTGTGGGTCCAACACTTGCGGCCACG
GTGGAGGGGCATCTGAGCCAGGCCTCAGAGAGTGGCGGGGGAAGTTGGGTGGGGA
AGTGTGCCCTTCTCATTCCTCTGAGGCTCATCCTCTTGGTGCCTCTCTTTCATGGAAA
GGGATAATAAGGTTATTGTGAGGATCCCCTGAGTTCGTATATTCAGACGCTTAGACA
GAGCCAGGCACAGAGAAGGGCCCGGGGTTGGCTAGTTTGATTGCTGGTGTAATTGCT
AATATCTTCCAGTTTGTATTGGTCAAGGTTCTGCAGAGAAGCAGAACCAGTAGGATG
TATATATTAAGAGTTTCAAGCTCATGTGACCGTGCGGGCTGGCAAGTCTGAAATCCG
CAGGGCAGGCCAGGCAGGCTGGCAATTCCTGCAGAATTTGATGTTGCAATACTGAGT
CCTAAGGCAGTCCTGGGGCAGAATTCCTTCTTCCCTGGGAGGCCTCAGTCTGTTCTCT
TAAGGCCTTCAACTGATTAAATGAGGCCTGCCCAAGTTATAGAGAGTAACCTGCCTT
ACTCCGTCTTCTGATTTAAATGTTAGTCACATCTAAAAAATATTTTCGCAGCAGCATT
TCCACTGGCTTTTGACCAAACATCAGGCCACAAAGTTGATCCCCAAAATTAACCATC
ACTCTGTGCCTGTAAGGGAGGGGCTGGGAAAGGGGAGCAGGTCTCCCCAAGGGGTG
ACCTTGGCTTTGTTCCTCCCAGGCCTGGAGTTTATTCGGAAAAGCCAGCTGGTCCAG
CCTGTGGGGCCACTGGTGGTGCTGCTGCCCCTGGCGGGTGGGTACAGCCGCGTCCTC
AACGCCGCCTGCCAGCGCCTGGCGAGGGCTGGGGTCGTGCTGGTCACCGCTGCCGG
CAACTTCCGGGACGATGCCTGCCTCTACTCCCCAGCCTCAGCTCCCGAGGTAGGTGC
TGGGGCTGCTGCCCCAAGGCGCGGGTAGGGGGCGGAGGGCGGAGGGCGGAGGGAG
GGCGGGCGGGCAGGCGGGCTTCTTGTGGCACGTGGGCTTCTTGTGGCACGTTCCTGG
AGGCCGAACCCTTCTGGCTTTGGAAGGAGTCGTCAGAGACCCCCGCCATGCGGGAG
GCTGGGGAGGAAGGGGCTCGAAACCTCCATCATCGCAGAGTCTGAATAGCAGTGGC
CCCGCCATGCGCCCACGTAGCGGCGCCTACGTAGCCACGCCCCCACACCCCGTCCTG
GCCACTCTCCCTCCTGAAGGTCTTCTGGTACCCGCCCCCTCCCCATCTCCATCCCCAG
GCCCTGCGTCCTCTGCCCAATACTCTTTGGGCCTCCCTGTTGTCCAGCTCTCTCCGCG
GCTCCATGACTGACAACTTGAGCAAGGCTAATGTGAATGGGAGCGGTTGAGGGCTC
AGACCTCTCACCCGAGGAACATCCACAGAGTGTGCCGCATGCCCGGTGCAGTGTGG
CTGCGGGGACACAGACACGGAGCCTCGGCCCTGAGGAGCTGGGGGGCAGTGACCGT
CCCTCCTCTGACCCACCACTCCTCCAGTGTCAGGACACTGCGGGTATCTAGGGGAAG
GAATCTTGTTCCACTTCAAGTCTGGAACTTCAAGTCTGTGTGTGTGCGTGCGCGCGC
GCGCGTTGGGGGTGGGGGTTGCAGAGCAGATGCGTACCTGACAGCGGTAACCTAGG
TCCCCCCTGGCCTATCAAGGCTTCCCTGGCGGCCGAATTTAAAGGCATCAAGCAAAC
AAAGCCCAACACATCTCTGCCTTGTCCTCTCAGTTTCCCCCCGTGGCACTTAGAACCA
CTTGATACACCGAATAGTTTCCTATCTCCCCCACTAGGATGTAAACTCCACAGGGGC
ATTGGGAATGCTGCCTGGCTATGGTAGGGACAGAGGGGAGCACCAGGGCGGGGCAG
GGGTGCCAGAGTTCTGCCTGGGCAGTCAGATTTTCCTTAGGAGGGGACATTTGAGTG
GGACCCAAACAGGTGTATAGCAGTTGTCCAGCCCAGCTGGCAAGGCCTGAGTCTGC
CTCTGCAACCCCTCTCTTGGGCTCCTTTCTCTGCCACCCACCTCCTCACCTTTCCAGGT
CATCACAGTTGGGGCCACCAATGCCCAAGACCAGCCGGTGACCCTGGGGACTTTGG
GGACCAACTTTGGCCGCTGTGTGGACCTCTTTGCCCCAGGGGAGGACATCATTGGTG
CCTCCAGCGACTGCAGCACCTGCTTTGTGTCACAGAGTGGGACATCACAGGCTGCTG
CCCACGTGGCTGGTAAGTCACCACCCCACTGCCTCGGCCACCGTGATGCTAACAGCC
CCTTTGGCAGTCAGGGTCTGTGCCGGGACCTCCAGTGCCAGGCTCTGTGCAGGGGGA
CCAGAGATGAAGTAGGCCTGATGGTGCCTTCAAGGACACTCAGTCTGATGAGGGAG
GCGAGTGCACAGAGGAAACACGAGGTCAGGGCTGTATTAGAGGGAGCCCAGAGGA
GGCACCTGCCCAGCCCGAGGGTCAGAGAAGGCATCTTGGAGGAGGGACATTTGATC
GGGAGCTTGATGGATGAATAGGAGTTCACCTGGCCGATAAGACAGCAACTACCAAG
GCTTAGAGGTGTGAGAGGAGGCTGTCTTACCTCACTGAGTAAGGACTGCAGGCGGC
TTACCTTCGAGAAGAGAGCTTAGTGTCTGTGTGCACGTGTGTTTGTGTGTATGTGTGT
GCGTGTGTGCACTGGCAGGAGTCCCCTGCTGGGGCAGGAGGGCCGGGCCATCACCA
TCTTTCACCATTCACCCCTGCACCAGGCATTGCAGCCATGATGCTGTCTGCCGAGCC
GGAGCTCACCCTGGCCGAGTTGAGGCAGAGACTGATCCACTTCTCTGCCAAAGATGT
CATCAATGAGGCCTGGTTCCCTGAGGACCAGCGGGTACTGACCCCCAACCTGGTGGC
CGCCCTGCCCCCCAGCACCCATGGGGCAGGTAAGCAGGATGGCAGGGTGGGCAAGT
CCAGGCTGGGGCTTGGGAGGTCTGTGTGACCTTGACAGTCTCTCCCTTCTCCCTTGTC
TGTGTAAGGAGGATGACGCCACCTTAAATAGGATTAAATGAGAATGGGGCTCTGAA
AGGGCTGTGCAATATTTTCATAACGTGTTTTTATAGAGACAGTTGAGTATGTTCTTTA
AGCCCTCCTCTCTCCTACCATGAACTAAAGATTTCTGTGGAGGTCCCCTCACTCCCAG
CACCCCCTCCTCATCCCAGGCCCTTTTTGCAGGTTGGCAGCTGTTTTGCAGGACTGTA
TGGTCAGCACACTCGGGGCCTACACGGATGGCCACAGCCGTCGCCCGCTGCGCCCCA
GATGAGGAGCTGCTGAGCTGCTCCAGTTTCTCCAGGAGTGGGAAGCGGCGGGGCGA
GCGCATGGAGGTGACTGTACCCCTCCTTCGTGTGTGTGTGTGTGTGTGTGTGTGTGTG
TGTGTGTGTGTGTGTGTGTGTGTCAGTGCTGGGCCCTCAGGGACCCCCAGCAAGCCC
CTCCATCCTCCAGACTCCAGCTCTTCTGTAAGCTTACAGGGCTGGCCAGACCAGGAG
TGGGGCACTCCTCACTTCACGCGGCTGGGGGCTGCTGGAGAGAGCCACAGCGGGAA
GGGTTTCCTAGAGGCTGCAGGACAGTGCTGGATGGATTTTCAATGCTCACCTGGGTG
TGAGCGTGCGGCAGGGCCGCGTGAGGGTCAGCGATCTGCTACTCTGGACTCAGCCAT
CTCTAGGCCCCTCTCACTCAGGTGCTCCATGGTTCTGGGAGCTGAGAAATCTCAAAC
CAGCAAAAAAGTGGAATTGATGTTGATGCTACAGGATAGTGCACAGATGCCATCTG
GTTGCAGCATTTTGGTGGAAGGGCAGTGCCCAGCTAGGAGAGTGAGGAGGGGCAGG
CATTTCTGGCTTGAGGAGATGGGGTCTTAATGCTCGTGTGAGAGGCAGAGTGGGTGG
AGTGGAGCTGGCTGGATCCTTGCTTTGGCCTCCTGGATTTCTCTCTATCTCCATTTTG
AAACCACTCTGTGTTTGGAAGAACTTTTGAGTATTCAGAGCTGCCCACTGGCAGAAC
AGTCTTCCTTGGGCAGGAGTGAGCTCCTTGTCCCCAGAAGGCTGGGTCTGGCTGGCC
CCTGGCAGGGACACTGATGAGGGTGCTTGAGTTGATCCTGTCTAGTCCCTTTCTGTGT
TTTCAAAGCCCATTCTAAAGCAGATTCCCATTTCCGTCTTTGACTCTAAGGCCCAAGG
GGGCAAGCTGGTCTGCCGGGCCCACAACGCTTTTGGGGGTGAGGGTGTCTACGCCAT
TGCCAGGTGCTGCCTGCTACCCCAGGCCAACTGCAGCGTCCACACAGCTCCACCAGC
TGAGGCCAGCATGGGGACCCGTGTCCACTGCCACCAACAGGGCCACGTCCTCACAG
GTAGGAGGCTGGGCTTGCCCTGGGGTGAGGAGGGGTCTCTTTCTCCTTATGCACCCA
CTGCCCGCGAGGCTTGGTCCTCACAAGTGTGATCCATGAGACTCAAGCCTGACTTGC
AGTTCCATACTCTGGTTCTGCCACTTCCATGCCCTTTGAGCCTGGGCAGGTGACCTTA
CTTCTCCTCATCTCAGCTTCCTCCTCCATAAGAGGGAAAAAGGTATTACCTGCCTCAT
TGTGTTGCAAGGAGATGGGCAGCATCTAGGGCACTGGCCTGGAGTATCGCAGGTGC
TTTGCCTAAGGTGGTGCAGTCCAGGAGAGGCAGCTCCAGAGAGAGGCCCCCGGCTG
GGGCTGAAAGGAGGGCAGACCTCGGTTTGAATTTCACCCTGCCGCTCTATAGCTGTG
TGACTTGGGCAAATTACTTAACATCTCTGTATGAGGAAATGATGAGTGCTAAGCACT
TAGCTTAGTGCCGGGACAATATAAATTCTAGCTATCGTTACTATTGTTTTCATCACCC
GTTGCTTTAAAATCCAGCCTCTGGTATAGGCAACTATTGACGGGCTACCCTGTGTCG
AAAACATGCCCAGGCAGGTAGCAGGAAGTCACAGATGGGGACCTCTTGGGGCATCA
AGGGATGGTGCCCTGAGGCTGAGCTGTTCTGGTTGGGTGGAGCATGAGAGGTCTGG
GAAGACAGTGGGACTCCAGCCTGGAATAAGAGGCTCAGAGTTGATTCTCGTCTGAG
CACGTCCAGGGGAACCACTGAGGGTTTGGGAACAGGAGAGTGAGGGTGAGAACCTG
GTTCTGGGCACAGCAGGCTGGCATGTAGGATGGATGTTCAGGAAAGATGAGCATAG
TCAGGTGGCTGGTGCCCTTGTCCAGGGGAGAGGCTCCGTCAGGTTCAGGGGTCCTGG
CTTGGAGGGAAGTCCGCCATGCTCTAATCACGCTCCCCTTTGGAAGTGCTCAGCCGA
TGAGCTCACAGGCACATGTCAGTTTGAAGTCATGGAATCTGACTCCATGAAGCGCAC
CTCAAAGAGCACCATTTTGCAGCTAAGGGAACTGCAGGCTGGACATGCTGAGTGGC
TGCCCCGAGCCCTTGCAGCTAGGACATAGAGAATGCTAGTAACCACAACCCTACCAT
GTTCAGAGCACATGCCAGGCTCCATGCTGGGGCTTCGCACGTGTCATCTTCACAGTG
TCCCTGTGAGTAGGTGTGGTTTCTCTTTCCATCTTACAAATGAGTAAACAGAGCCTCA
GTGTAGCTAAGTAACCACTATTTTAGGTTTCTTAGCCAATGGGTGTGTCTGACTCCTA
AGCCCATGGAGGGCATTCTGAGGTGGTTCAGACAGACCCCGGCTTACCCTTGAACTT
CTGCCTGCTGGCTGCATAGGGAGGGGCTGGGGGGAGTTTGAGCATCTCAGGCCATA
GAGCCCCTGCCTCACTGTCTCCATCTCTGGGTGGAAAGATGGTGTTTTCCCTGAGAA
ACTAAGGCTCAGAGAGGTTGAATGGCTCTCCCAAGGTCACACAGCTGGTCAGCTGC
AGAGTTGAGAACACAGGAGTCCTGGTGCTCAGGCCAGCATCTCTTTTTTTCTTTGAG
TTGTTTCTAGGTTTCCTAGCTCTTGCCTCAGACCTTAAAGAGAGAGGGTCTGATGGG
GATGGGCACTGGAGACGGAGCATCCCAGCATTTCACATCTGAGCTGGCTTTCCTCTG
CCCCAGGCTGCAGCTCCCACTGGGAGGTGGAGGACCTTGGCACCCACAAGCCGCCT
GTGCTGAGGCCACGAGGTCAGCCCAACCAGTGCGTGGGCCACAGGGAGGCCAGCAT
CCACGCTTCCTGCTGCCATGCCCCAGGTCTGGAATGCAAAGTCAAGGAGCATGGAAT
CCCGGCCCCTCAGGAGCAGGTGAAGAGGCCCGTGAGGCCGGGTGGGTGGGGTGCTG
CGTGTCTCTCCTGCACAGCTTTTCTGTGTCAGTTTGTGCCACCACCATACCGCCATGC
ATCAGGGTGGCGGTTTGCCAGGTAGATGCTGTGGGCAGCTTCCGCCATTGTGTGGAC
AGCATGTATATGTGTCTCTGTGTGGCTGGGTCTGTTTTTGCTTTTGTCCAGATCAGTA
AGGTTTGCTACCTGGGTACCCCACTCCACTTGGAGTAGAATGTGCATAAATATGGCA
TAAAGAAATGCAATATGCATGCATTTATTGATTGATCTATTTTTTTCTGAGATGGGGT
CTTGCTGTGTTGCCCAGGCTGGTCTCAAATTCCTGGGCTCAAGCAATCCTCTGGTCTC
AGCCTCCCCAAGTGTTGGGATTATAGGCATGAGCCGCTGCACCTGGCCTCTCTGATC
TATTTAACAAACCTGCTGGGAGGGTCTCAGGGTCAGGAGCAGCACTGGGCTCTGAG
GACACAGAGCTCACTCAGCCGTGACCCAGAGGGGGTGCCTGAGCTGCATGCTGAAG
GTTGTTAGCATGACCAGCAAGGCAAGAAAAGGCCCTGCCGAGATTAGCAAGGCATG
TGCCAAGCCCTGGAATGTGACAGCCGGGCCTTCTAGAAACCTGAGTGTATAACTCTC
CTTAAAAGCCAGTAGGAGCTCCTCAAAAGGCAGCCCTAAGGAGTCCACTCTTAAAT
GAACTCAGAGTCAGTTTTAAAATGCAAGTCTGTGTTGATTCTGGTCTGGATGGTGCA
TTCCTCGAGAGCAAAAGACAGTCTTGGTCTTGGATCCACTTGCCCTGGGTACACTGA
GGGCTGCTAGGTTCCAGGTGCTCTTCCTGGCACTGGGGAGGGATACAGGCCCAAGA
GACATGCTGTTCTCCCTCCTGGAGCATCTATTTTAGTGGAGGAAGACAGAAAACAAA
CCATTAATATAGAGTACTGAAAAGATGCGATGGAGAAAACTATAGCAAGGAAGGGA
ATGGGGTGGGAGAGAGGTCAGGAGAGGTCTCGCTGACAAGGTGGACGAAACAGGC
CATGAGGCAGAGAACATGTTCCAGGCAAAGCAAAGGCCCCCAGGTGGGGATGTGCA
GGGAGTACCAGGAAACCAGAGAGGTGGGAATAGTTATGAGATGGGGGGTGCCTCAG
AGGGGACAGGGCCAAGTCAGGTGAGACCTGAGGGTCACAGTCAGCAGTGAGCTGGG
GCCATGCAGGGGTCTGGCCTCAGAGGAGTGTGGTCTGGCCTGGATCTGAACCTCTCA
CTGTGGCCTAGCTGCTGAGCTGAGAAGAGATGACAAGGACCTTGGGCAGAAGCAGG
GAGACTGGAGGGAGGCGGTGGAGGGTCCAGGCGTTGGGGGGGGCTCAGGCTGGA
GTCTGAAGGGAGCCTGCAGGCCTGGTGGGTGGATGTGGGTGGGAGAGGGGGAGGAT
GGCACCAAGGCTCGGGCCCCTGGACAGATGGAGTTGCCATTAAGTGGGATGGGGCA
GGCTATGGGGCCATCAGTTTCAGAGGGATGAGTTTGGCACTGGCATGGTAGGCATCT
GTCTATCTCCACGGCCCTCAAACCAGGCATGAAGCAGGAGCTCACGTGTTTGGTCAG
CCATGGTGCAGAACCGCCTGGGTGGGAGGTGCGGGGTGGGAGATACACGGTTGTGT
CCCAAATGGGCTCTGAGCCAGCGAGGGCCGTCTGCACTTTGGCCTCACAGAAGGAT
GTCGGAGGGAGAAATGAAGTGTGGGTGGGGGTCCCGGGCCACGCTAGACATGTGCT
TTCTTTTCCTCGGGCTCTGGCAGGTGACCGTGGCCTGCGAGGAGGGCTGGACCCTGA
CTGGCTGCAGTGCCCTCCCTGGGACCTCCCACGTCCTGGGGGCCTACGCCGTAGACA
ACACGTGTGTAGTCAGGAGCCGGGACGTCAGCACTACAGGCAGCACCAGCGAAGGG
GCCGTGACAGCCGTTGCCATCTGCTGCCGGAGCCGGCACCTGGCGCAGGCCTCCCAG
GAGCTCCAGTGACAGCCCCATCCCAGGATGGGTGTCTGGGGAGGGTCAAGGGCTGG
GGCTGAGCTTTAAAATGGTTCCGACTTGTCCCTCTCTCAGCCCTCCATGGCCTGGCAC
GAGGGGATGGGGATGCTTCCGCCTTTCCGGGGCTGCTGGCCTGGCCCTTGAGTGGGG
CAGCCTCCTTGCCTGGAACTCACTCACTCTGGGTGCCTCCTCCCCAGGTGGAGGTGC
CAGGAAGCTCCCTCCCTCACTGTGGGGCATTTCACCATTCAAACAGGTCGAGCTGTG
CTCGGGTGCTGCCAGCTGCTCCCAATGTGCCGATGTCCGTGGGCAGAATGACTTTTA
TTGAGCTCTTGTTCCGTGCCAGGCATTCAATCCTCAGGTCTCCACCAAGGAGGCAGG
ATTCTTCCCATGGATAGGGGAGGGGGCGGTAGGGGCTGCAGGGACAAACATCGTTG
GGGGGTGAGTGTGAAAGGTGCTGATGGCCCTCATCTCCAGCTAACTGTGGAGAAGC
CCCTGGGGGCTCCCTGATTAATGGAGGCTTAGCTTTCTGGATGGCATCTAGCCAGAG
GCTGGAGACAGGTGCGCCCCTGGTGGTCACAGGCTGTGCCTTGGTTTCCTGAGCCAC
CTTTACTCTGCTCTATGCCAGGCTGTGCTAGCAACACCCAAAGGTGGCCTGCGGGGA
GCCATCACCTAGGACTGACTCGGCAGTGTGCAGTGGTGCATGCACTGTCTCAGCCAA
CCCGCTCCACTACCCGGCAGGGTACACATTCGCACCCCTACTTCACAGAGGAAGAAA
CCTGGAACCAGAGGGGGCGTGCCTGCCAAGCTCACACAGCAGGAACTGAGCCAGAA
ACGCAGATTGGGCTGGCTCTGAAGCCAAGCCTCTTCTTACTTCACCCGGCTGGGCTC
CTCATTTTTACGGGTAACAGTGAGGCTGGGAAGGGGAACACAGACCAGGAAGCTCG
GTGAGTGATGGCAGAACGATGCCTGCAGGCATGGAACTTTTTCCGTTATCACCCAGG
CCTGATTCACTGGCCTGGCGGAGATGCTTCTAAGGCATGGTCGGGGGAGAGGGCCA
ACAACTGTCCCTCCTTGAGCACCAGCCCCACCCAAGCAAGCAGACATTTATCTTTTG
GGTCTGTCCTCTCTGTTGCCTTTTTACAGCCAACTTTTCTAGACCTGTTTTGCTTTTGT
AACTTGAAGATATTTATTCTGGGTTTTGTAGCATTTTTATTAATATGGTGACTTTTTA
AAATAAAAACAAACAAACGTTGTCCTAAC
Human PCSK9 Amino Acid Sequence (NP_777596.2)
(SEQ ID NO: 756)
MGTVSSRRSWWPLPLLLLLLLLLGPAGARAQEDEDGDYEELVLALRSEEDGLAEAPEH
GTTATFHRCAKDPWRLPGTYVVVLKEETHLSQSERTARRLQAQAARRGYLTKILHVFH
GLLPGFLVKMSGDLLELALKLPHVDYIEEDSSVFAQSIPWNLERITPPRYRADEYQPPDG
GSLVEVYLLDTSIQSDHREIEGRVMVTDFENVPEEDGTRFHRQASKCDSHGTHLAGVVS
GRDAGVAKGASMRSLRVLNCQGKGTVSGTLIGLEFIRKSQLVQPVGPLVVLLPLAGGYS
RVLNAACQRLARAGVVLVTAAGNFRDDACLYSPASAPEVITVGATNAQDQPVTLGTLG
TNFGRCVDLFAPGEDIIGASSDCSTCFVSQSGTSQAAAHVAGIAAMMLSAEPELTLAELR
QRLIHFSAKDVINEAWFPEDQRVLTPNLVAALPPSTHGAGWQLFCRTVWSAHSGPTRM
ATAVARCAPDEELLSCSSFSRSGKRRGERMEAQGGKLVCRAHNAFGGEGVYAIARCCL
LPQANCSVHTAPPAEASMGTRVHCHQQGHVLTGCSSHWEVEDLGTHKPPVLRPRGQPN
QCVGHREASIHASCCHAPGLECKVKEHGIPAPQEQVTVACEEGWTLTGCSALPGTSHVL
GAYAVDNTCVVRSRDVSTTGSTSEGAVTAVAICCRSRHLAQASQELQ

### Base Editor Protein-gRNA Complexes

In another aspect, provided herein is a complex comprising the single guide RNA as provided herein in complex with the base editor fusion protein, e.g. the adenosine base editor fusion protein, wherein the complex comprises increased stability as compared to a complex with an unmodified single guide RNA and a base editor protein, wherein the stability is measured by half life of the complex ex vivo or in vitro.

In some embodiments, the complex comprises increased stability as compared to a complex with an unmodified single guide RNA and a Cas9 protein. In some embodiments, the complex comprises increased stability by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas9 protein. In some embodiments, the complex comprises increased stability by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas9 protein.

In some embodiments, wherein the stability of the complex is measured by half life of the complex. In some embodiments, wherein the stability of the complex is measured by half life of the complex ex vivo. In some embodiments, wherein the stability of the complex is measured by half life of the complex in vitro.

In some embodiments, the complex comprises increased half life by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas9 protein wherein half life of the complex is measured ex vivo. In some embodiments, the single guide RNA exhibits increased half life of the complex by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas9 protein, wherein half life of the complex is measured ex vivo.

In some embodiments, the complex comprises increased half life when measured in vitro by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to a complex with an unmodified single guide RNA and a Cas9 protein, wherein half life of the complex is measured in vitro. In some embodiments, the single guide RNA exhibits increased half life of the complex by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to a complex with an unmodified single guide RNA and a Cas9 protein, wherein half life of the complex is measured in vitro.

In another aspect, provided herein is a cell comprising the complex as provided herein. In some embodiments, the cell may be an in vitro cell. In some embodiments, the cell may be an ex vivo cell. In some embodiments, the cell may be an in vivo cell. In some embodiments, the cell may be an isolated cell.

### Pharmaceutical Compositions

In some aspects, provided herein is a pharmaceutical composition comprising the gene editing system as provided herein and a pharmaceutically acceptable carrier or excipient.

In some aspects, provided herein, is a pharmaceutical composition comprising a guide nucleic acid described herein and a gene editor protein or a nucleic acid sequence encoding the gene editor protein and a pharmaceutically acceptable carrier. The gene editing system comprising a guide nucleic acid sequence described herein and a gene editor protein or a nucleic acid sequence encoding the gene editor protein can be formulated into pharmaceutical compositions. Pharmaceutical compositions are formulated in a conventional manner using one or more pharmaceutically acceptable inactive ingredients that facilitate processing of the active compounds into preparations that can be used pharmaceutically. Suitable formulations for use in the present disclosure and methods of delivery are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can include a guide nucleic acid sequence described herein and a gene editor protein or a nucleic acid sequence encoding the gene editor protein with one or more of other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the guide nucleic acid sequence described herein and the gene editor protein or a nucleic acid sequence encoding the gene editor protein to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. The pharmaceutical compositions can be administered by intraperitoneal injection, intramuscular injection, subcutaneous injection, or intravenous injection of the subject. The pharmaceutical compositions can be administered parenterally, intravenously, intramuscularly, or orally.

For administration by inhalation, the gene editing systems described herein can be formulated for use as an aerosol, a mist, or a powder. For buccal or sublingual administration, the pharmaceutical compositions may be formulated in the form of tablets, lozenges, or gels formulated in a conventional manner. The pharmaceutical compositions described herein can be prepared as transdermal dosage forms. The gene editing systems described herein can be formulated into a pharmaceutical composition suitable for intramuscular, subcutaneous, or intravenous injection. The gene editing systems described herein can be administered topically and can be formulated into a variety of topically administrable compositions, such as solutions, suspensions, lotions, gels, pastes, medicated sticks, balms, creams, or ointments. The gene editing systems described herein can be formulated in rectal compositions such as enemas, rectal gels, rectal foams, rectal aerosols, suppositories, jelly suppositories, or retention enemas. The gene editing systems described herein can be formulated for oral administration such as a tablet, a capsule, or liquid in the form of aqueous suspensions or solutions selected from the group including, but not limited to, aqueous oral dispersions, emulsions, solutions, elixirs, gels, and syrups.

The pharmaceutical composition comprising a guide nucleic acid sequence described herein and a gene editor protein or a nucleic acid sequence encoding the gene editor protein further comprises a therapeutic agent. The additional therapeutic agent may modulate different aspects of the disease, disorder, or condition being treated and provide a greater overall benefit than administration of either the replication competent recombinant adenovirus or the therapeutic agent alone. Therapeutic agents include, but are not limited to, a chemotherapeutic agent, a radiotherapeutic agent, a hormonal therapeutic agent, and/or an immunotherapeutic agent. The therapeutic agent may be a radiotherapeutic agent. The therapeutic agent may be a hormonal therapeutic agent. The therapeutic agent may be an immunotherapeutic agent. The therapeutic agent can be a chemotherapeutic agent. Preparation and dosing schedules for additional therapeutic agents can be used according to manufacturers' instructions or as determined empirically by a skilled practitioner. For example, preparation and dosing schedules for chemotherapy are also described in The Chemotherapy Source Book, 4th Edition, 2008, M. C. Perry, Editor, Lippincott, Williams & Wilkins, Philadelphia, PA.

The subjects that can be treated with gene editing systems of guide nucleic acid sequences described herein and a gene editor protein or a nucleic acid sequence encoding the gene editor protein and methods described herein can be any subject with a disease or a condition. For example, the subject may be a eukaryotic subject, such as an animal. The subject can be a mammal, e.g., human. The subject can be a human. The subject can be a non-human animal. The subject can be a fetus, an embryo, or a child. The subject can be a non-human primate such as chimpanzee, and other apes and monkey species; farm animals such as cattle, horses, sheep, goats, pigs; domestic animals such as rabbits, dogs, and cats; laboratory animals including rodents, such as rats, mice, and guinea pigs, and the like.

The subject can be prenatal (e.g., a fetus), a child (e.g., a neonate, an infant, a toddler, a preadolescent), an adolescent, a pubescent, or an adult (e.g., an early adult, a middle-aged adult, a senior citizen). The human subject can be between about 0 month and about 120 years old, or older. The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 years old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subjects can include male subjects and/or female subjects.

In some embodiments, the guide RNA and the mRNA are administered at a total amount which results in a plasma Cmax of the iLipid in the human subject is about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78. 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195,200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 μg/mL. In some embodiments, the guide RNA and the mRNA are administered at a total amount which results in a plasma Cmax of the iLipid in the human subject is about 0.1 μg/mL to about 1000 μg/mL, about 0.2 μg/mL to about 900 μg/mL, about 0.3 μg/mL to about 800 μg/mL, about 0.4 μg/mL to about 700 μg/mL, about 0.5 μg/mL to about 600 μg/mL, about 0.6 μg/mL to about 500 μg/mL, about 0.7 μg/mL to about 400 μg/mL, about 0.8 μg/mL to about 300 μg/mL, about 0.9 μg/mL to about 200 μg/mL, 1 μg/mL to about 100 μg/mL, about 2 μg/mL to about 90 μg/mL, about 3 μg/mL to about 80 μg/mL, about 4 μg/mL to about 70 μg/mL, about 5 μg/mL to about 60 μg/mL, about 6 μg/mL to about 50 μg/mL, about 7 μg/mL to about 40 μg/mL, about 8 μg/mL to about 30 μg/mL, or about 9 μg/mL to about 20 μg/mL.

In some embodiments, the guide RNA and the mRNA are administered at a total amount which results in a plasma Cmax of the PEG lipid in the human subject to be about 0.01 μg/mL to about 300 μg/mL, about 0.02 μg/mL to about 250 μg/mL, about 0.03 μg/mL to about 200 μg/mL, about 0.04 μg/mL to about 150 μg/mL, about 0.05 μg/mL to about 100 μg/mL, about 0.06 μg/mL to about 90 μg/mL, about 0.07 μg/mL to about 80 μg/mL, about 0.08 μg/mL to about 70 μg/mL, about 0.09 μg/mL to about 60 μg/mL, about 0.1 μg/mL to about 50 μg/mL, about 0.2 μg/mL to about 45 μg/mL, about 0.3 μg/mL to about 40 μg/mL, about 0.4 μg/mL to about 35 μg/mL, about 0.5 μg/mL to about 30 μg/mL, about 0.6 μg/mL to about 25 μg/mL, about 0.7 μg/mL to about 20 μg/mL, about 0.8 μg/mL to about 19 μg/mL, about 0.9 μg/mL to about 17 μg/mL, about 1 μg/mL to about 15 μg/mL, about 2 μg/mL to about 14 μg/mL, about 3 μg/mL to about 13 μg/mL, about 4 μg/mL to about 12 μg/mL, about 5 μg/mL to about 11 μg/mL, about 6 μg/mL to about 10 μg/mL, or about 7 μg/mL to about 9 μg/mL.

In some embodiments, the guide RNA and the mRNA are administered at a total amount which results in a plasma Cmax of the mRNA in the human subject to be about 0.01 μg/mL to about 5 μg/mL, about 0.02 μg/mL to about 5 μg/mL, about 0.03 μg/mL to about 5 μg/mL, about 0.04 μg/mL to about 5 μg/mL, about 0.05 μg/mL to about 5 μg/mL, about 0.06 μg/mL to about 5 μg/mL, about 0.07 μg/mL to about 5 μg/mL, about 0.08 μg/mL to about 5 μg/mL, about 0.09 μg/mL to about 5 μg/mL, about 0.1 μg/mL to about 5 μg/mL, about 0.2 μg/mL to about 5 μg/mL, about 0.3 μg/mL to about 5 μg/mL, about 0.4 μg/mL to about 5 μg/mL, about 0.5 μg/mL to about 5 μg/mL, about 0.6 μg/mL to about 5 μg/mL, about 0.7 μg/mL to about 5 μg/mL, about 0.8 μg/mL to about 5 μg/mL, 0.9 μg/mL to about 5 μg/mL, about 1 μg/mL to about 5 μg/mL, about 1.5 μg/mL to about 5 μg/mL, about 2 μg/mL to about 5 μg/mL, about 2.5 μg/mL to about 5 μg/mL, about 3 μg/mL to about 5 μg/mL, about 3.5 μg/mL to about 5 μg/mL, about 4 μg/mL to about 5 μg/mL, or about 4.5 μg/mL to about 5 μg/mL.

In some embodiments, the guide RNA and the mRNA are administered at a total amount which results in an area under the plasma concentration-time curve (AUC) of the iLipid in the human subject to be about 5 μg×h/mL to about 60000 μg×h/mL, about 10 μg×h/mL to about 55000 μg×h/mL, about 20 μg×h/mL to about 50000 μg×h/mL, about 30 μg×h/mL to about 45000 μg×h/mL, about 40 μg×h/mL to about 40000 μg×h/mL, about 50 μg×h/mL to about 35000 μg×h/mL, about 60 μg×h/mL to about 30000 μg×h/mL, about 70 μg×h/mL to about 25000 μg×h/mL, about 80 μg×h/mL to about 20000 μg×h/mL, about 90 μg×h/mL to about 15000 μg×h/mL, about 100 μg×h/mL to about 10000 μg×h/mL, about 200 μg×h/mL to about 9000 μg×h/mL, about 300 μg×h/mL to about 8000 μg×h/mL, about 400 μg×h/mL to about 7000 μg×h/mL, about 500 μg×h/mL to about 6000 μg×h/mL, about 600 μg×h/mL to about 5000 μg×h/mL, about 700 μg×h/mL to about 4000 μg×h/mL, about 800 μg×h/mL to about 3000 μg×h/mL, or about 900 μg×h/mL to about 2000 μg×h/mL.

In some embodiments, the guide RNA and the mRNA are administered at a total amount which results in an AUC of the PEG lipid in the human subject to be about 1 μg×h/mL to about 9500 μg×h/mL, about 2 μg×h/mL to about 9000 μg×h/mL, about 3 μg×h/mL to about 8500 μg×h/mL, about 4 μg×h/mL to about 8000 μg×h/mL, about 5 μg×h/mL to about 7500 μg×h/mL, about 6 μg×h/mL to about 7000 μg×h/mL, about 7 μg×h/mL to about 6500 μg×h/mL, about 8 μg×h/mL to about 6000 μg×h/mL, about 9 μg×h/mL to about 5500 μg×h/mL, about 10 μg×h/mL to about 5000 μg×h/mL, about 20 μg×h/mL to about 4500 μg×h/mL, about 30 μg×h/mL to about 4000 μg×h/mL, about 40 μg×h/mL to about 3500 μg×h/mL, about 50 μg×h/mL to about 3000 μg×h/mL, about 60 μg×h/mL to about 2500 μg×h/mL, about 70 μg×h/mL to about 2000 μg×h/mL, about 80 μg×h/mL to about 1500 μg×h/mL, about 90 μg×h/mL to about 1000 μg×h/mL, about 100 μg×h/mL to about 900 μg×h/mL, about 200 μg×h/mL to about 800 μg×h/mL, about 300 μg×h/mL to about 700 μg×h/mL, or about 400 μg×h/mL to about 600 μg×h/mL.

In some embodiments, the guide RNA and the mRNA are administered at a total amount which results in an AUC of the PEG lipid in the human subject to be about 1 μg×h/mL to about 3000 μg×h/mL, about 10 μg×h/mL to about 2900 μg×h/mL, about 20 μg×h/mL to about 2800 μg×h/mL, about 30 μg×h/mL to about 2700 μg×h/mL, about 40 μg×h/mL to about 2600 μg×h/mL, about 50 μg×h/mL to about 2500 μg×h/mL, about 60 μg×h/mL to about 2400 μg×h/mL, about 70 μg×h/mL to about 2300 μg×h/mL, about 80 μg×h/mL to about 2200 μg×h/mL, about 90 μg×h/mL to about 2100 μg×h/mL, about 100 μg×h/mL to about 2000 μg×h/mL, about 200 μg×h/mL to about 1900 μg×h/mL, about 300 μg×h/mL to about 1800 μg×h/mL, about 400 μg×h/mL to about 1700 μg×h/mL, about 500 μg×h/mL to about 1600 μg×h/mL, 600 μg×h/mL to about 1500 μg×h/mL, 700 μg×h/mL to about 1400 μg×h/mL, 800 μg×h/mL to about 1300 μg×h/mL, 900 μg×h/mL to about 1200 μg×h/mL, or 1000 μg×h/mL to about 1100 μg×h/mL.

In some embodiments, the guide RNA and the mRNA are administered at a total amount which results in an AUC of the mRNA in the human subject to be about 0.1 μg×h/mL to about 1000 μg×h/mL, about 0.2 μg×h/mL to about 900 μg×h/mL, about 0.3 μg×h/mL to about 800 μg×h/mL, about 0.4 μg×h/mL to about 700 μg×h/mL, about 0.5 μg×h/mL to about 600 μg×h/mL, about 0.6 μg×h/mL to about 500 μg×h/mL, about 0.7 μg×h/mL to about 400 μg×h/mL, about 0.8 μg×h/mL to about 300 μg×h/mL, about 0.9 μg×h/mL to about 200 μg×h/mL, about 1 μg×h/mL to about 100 μg×h/mL, about 2 μg×h/mL to about 90 μg×h/mL, about 3 μg×h/mL to about 80 μg×h/mL, about 4 μg×h/mL to about 60 μg×h/mL, about 5 μg×h/mL to about 50 μg×h/mL, about 6 μg×h/mL to about 40 μg×h/mL, about 7 μg×h/mL to about 30 μg×h/mL, about 8 μg×h/mL to about 20 μg×h/mL, or about 9 μg×h/mL to about 10 μg×h/mL.

In some embodiments, the guide RNA and the mRNA are administered at the total amount of about 0.01 mg/kg to about 5 mg/kg, about 0.02 mg/kg to about 5 mg/kg, about 0.03 mg/kg to about 5 mg/kg, about 0.04 mg/kg to about 5 mg/kg, about 0.05 mg/kg to about 5 mg/kg, about 0.06 mg/kg to about 5 mg/kg, about 0.07 mg/kg to about 5 mg/kg, about 0.08 mg/kg to about 5 mg/kg, about 0.09 mg/kg to about 5 mg/kg, about 0.1 mg/kg to about 5 mg/kg, about 0.2 mg/kg to about 5 mg/kg, about 0.3 mg/kg to about 5 mg/kg, about 0.4 mg/kg to about 5 mg/kg, about 0.5 mg/kg to about 5 mg/kg, about 0.6 mg/kg to about 5 mg/kg, about 0.7 mg/kg to about 5 mg/kg, about 0.8 mg/kg to about 5 mg/kg, about 0.9 mg/kg to about 5 mg/kg, about 1 mg/kg to about 5 mg/kg, about 1.5 mg/kg to about 5 mg/kg, about 2 mg/kg to about 5 mg/kg, about 2.5 mg/kg to about 5 mg/kg, about 3 mg/kg to about 5 mg/kg, about 3.5 mg/kg to about 5 mg/kg, about 4 mg/kg to about 5 mg/kg, or about 4.5 mg/kg to about 5 mg/kg.

In some embodiments, the guide RNA and the mRNA are administered at the total amount of about 0.01 mg/kg to about 3 mg/kg, about 0.02 mg/kg to about 3 mg/kg, about 0.03 mg/kg to about 3 mg/kg, about 0.04 mg/kg to about 3 mg/kg, about 0.05 mg/kg to about 3 mg/kg, about 0.06 mg/kg to about 3 mg/kg, about 0.07 mg/kg to about 3 mg/kg, about 0.08 mg/kg to about 3 mg/kg, about 0.09 mg/kg to about 3 mg/kg, about 0.1 mg/kg to about 3 mg/kg, about 0.2 mg/kg to about 3 mg/kg, about 0.3 mg/kg to about 3 mg/kg, about 0.4 mg/kg to about 3 mg/kg, about 0.5 mg/kg to about 3 mg/kg, about 0.6 mg/kg to about 3 mg/kg, about 0.7 mg/kg to about 3 mg/kg, about 0.8 mg/kg to about 3 mg/kg, about 0.9 mg/kg to about 3 mg/kg, about 1 mg/kg to about 3 mg/kg, about 1.5 mg/kg to about 3 mg/kg, about 2 mg/kg to about 3 mg/kg, or about 2.5 mg/kg to about 3 mg/kg.

In some embodiments, the guide RNA and the mRNA are administered at the total amount of about 0.01 mg/kg to about 2 mg/kg, about 0.02 mg/kg to about 2 mg/kg, about 0.03 mg/kg to about 2 mg/kg, about 0.04 mg/kg to about 2 mg/kg, about 0.05 mg/kg to about 2 mg/kg, about 0.06 mg/kg to about 2 mg/kg, about 0.07 mg/kg to about 2 mg/kg, about 0.08 mg/kg to about 2 mg/kg, about 0.09 mg/kg to about 2 mg/kg, about 0.1 mg/kg to about 2 mg/kg, about 0.2 mg/kg to about 2 mg/kg, about 0.3 mg/kg to about 2 mg/kg, about 0.4 mg/kg to about 2 mg/kg, about 0.5 mg/kg to about 2 mg/kg, about 0.6 mg/kg to about 2 mg/kg, about 0.7 mg/kg to about 2 mg/kg, about 0.8 mg/kg to about 2 mg/kg, about 0.9 mg/kg to about 2 mg/kg, about 1 mg/kg to about 2 mg/kg, about 1.1 mg/kg to about 2 mg/kg, about 1.2 mg/kg to about 2 mg/kg, about 1.3 mg/kg to about 2 mg/kg, about 1.4 mg/kg to about 2 mg/kg, about 1.5 mg/kg to about 2 mg/kg, about 1.6 mg/kg to about 2 mg/kg, about 1.7 mg/kg to about 2 mg/kg, about 1.8 mg/kg to about 2 mg/kg, or about 1.9 mg/kg to about 2 mg/kg.

In some embodiments, the guide RNA and the mRNA are administered at the total amount of about 0.01 mg/kg to about 1.5 mg/kg, about 0.02 mg/kg to about 1.5 mg/kg, about 0.03 mg/kg to about 1.5 mg/kg, about 0.04 mg/kg to about 1.5 mg/kg, about 0.05 mg/kg to about 1.5 mg/kg, about 0.06 mg/kg to about 1.5 mg/kg, about 0.07 mg/kg to about 1.5 mg/kg, about 0.08 mg/kg to about 1.5 mg/kg, about 0.09 mg/kg to about 1.5 mg/kg, about 0.1 mg/kg to about 1.5 mg/kg, about 0.2 mg/kg to about 1.5 mg/kg, about 0.3 mg/kg to about 1.5 mg/kg, about 0.4 mg/kg to about 1.5 mg/kg, about 0.5 mg/kg to about 1.5 mg/kg, about 0.6 mg/kg to about 1.5 mg/kg, about 0.7 mg/kg to about 1.5 mg/kg, about 0.8 mg/kg to about 1.5 mg/kg, about 0.9 mg/kg to about 1.5 mg/kg, about 1 mg/kg to about 1.5 mg/kg, about 1.1 mg/kg to about 1.5 mg/kg, about 1.2 mg/kg to about 1.5 mg/kg, about 1.3 mg/kg to about 1.5 mg/kg, or about 1.4 mg/kg to about 1.5 mg/kg.

In some embodiments, the guide RNA and the mRNA are administered at the total amount of about 0.01 mg/kg to about 1.25 mg/kg, about 0.02 mg/kg to about 1.25 mg/kg, about 0.03 mg/kg to about 1.25 mg/kg, about 0.04 mg/kg to about 1.25 mg/kg, about 0.05 mg/kg to about 1.25 mg/kg, about 0.06 mg/kg to about 1.25 mg/kg, about 0.07 mg/kg to about 1.25 mg/kg, about 0.08 mg/kg to about 1.25 mg/kg, about 0.09 mg/kg to about 1.25 mg/kg, about 0.1 mg/kg to about 1.25 mg/kg, about 0.2 mg/kg to about 1.25 mg/kg, about 0.3 mg/kg to about 1.25 mg/kg, about 0.4 mg/kg to about 1.25 mg/kg, about 0.5 mg/kg to about 1.25 mg/kg, about 0.6 mg/kg to about 1.25 mg/kg, about 0.7 mg/kg to about 1.25 mg/kg, about 0.8 mg/kg to about 1.25 mg/kg, about 0.9 mg/kg to about 1.25 mg/kg, about 1 mg/kg to about 1.25 mg/kg, about 1.1 mg/kg to about 1.25 mg/kg, or about 1.2 mg/kg to about 1.25 mg/kg.

In some embodiments, the guide RNA and the mRNA are administered at the total amount of about 0.01 mg/kg to about 1 mg/kg, about 0.02 mg/kg to about 1 mg/kg, about 0.03 mg/kg to about 1 mg/kg, about 0.04 mg/kg to about 1 mg/kg, about 0.05 mg/kg to about 1 mg/kg, about 0.06 mg/kg to about 1 mg/kg, about 0.07 mg/kg to about 1 mg/kg, about 0.08 mg/kg to about 1 mg/kg, about 0.09 mg/kg to about 1 mg/kg, about 0.1 mg/kg to about 1 mg/kg, about 0.2 mg/kg to about 1 mg/kg, about 0.3 mg/kg to about 1 mg/kg, about 0.4 mg/kg to about 1 mg/kg, about 0.5 mg/kg to about 1 mg/kg, about 0.6 mg/kg to about 1 mg/kg, about 0.7 mg/kg to about 1 mg/kg, about 0.8 mg/kg to about 1 mg/kg, or about 0.9 mg/kg to about 1 mg/kg.

In some embodiments, the guide RNA and the mRNA are administered at the total amount of about 0.01 mg/kg to about 0.9 mg/kg, about 0.02 mg/kg to about 0.9 mg/kg, about 0.03 mg/kg to about 0.9 mg/kg, about 0.04 mg/kg to about 0.9 mg/kg, about 0.05 mg/kg to about 0.9 mg/kg, about 0.06 mg/kg to about 0.9 mg/kg, about 0.07 mg/kg to about 0.9 mg/kg, about 0.08 mg/kg to about 0.9 mg/kg, about 0.09 mg/kg to about 0.9 mg/kg, about 0.1 mg/kg to about 0.9 mg/kg, about 0.2 mg/kg to about 0.9 mg/kg, about 0.3 mg/kg to about 0.9 mg/kg, about 0.4 mg/kg to about 0.9 mg/kg, about 0.5 mg/kg to about 0.9 mg/kg, about 0.6 mg/kg to about 0.9 mg/kg, about 0.7 mg/kg to about 0.9 mg/kg, or about 0.8 mg/kg to about 0.9 mg/kg.

In some embodiments, the guide RNA and the mRNA are administered at the total amount of about 0.01 mg/kg to about 0.8 mg/kg, about 0.02 mg/kg to about 0.8 mg/kg, about 0.03 mg/kg to about 0.8 mg/kg, about 0.04 mg/kg to about 0.8 mg/kg, about 0.05 mg/kg to about 0.8 mg/kg, about 0.06 mg/kg to about 0.8 mg/kg, about 0.07 mg/kg to about 0.8 mg/kg, about 0.08 mg/kg to about 0.8 mg/kg, about 0.09 mg/kg to about 0.8 mg/kg, about 0.1 mg/kg to about 0.8 mg/kg, about 0.2 mg/kg to about 0.8 mg/kg, about 0.3 mg/kg to about 0.8 mg/kg, about 0.4 mg/kg to about 0.8 mg/kg, about 0.5 mg/kg to about 0.8 mg/kg, about 0.6 mg/kg to about 0.8 mg/kg, or about 0.7 mg/kg to about 0.8 mg/kg.

In some embodiments, the guide RNA and the mRNA are administered at the total amount of about 0.01 mg/kg to about 0.6 mg/kg, about 0.02 mg/kg to about 0.6 mg/kg, about 0.03 mg/kg to about 0.6 mg/kg, about 0.04 mg/kg to about 0.6 mg/kg, about 0.05 mg/kg to about 0.6 mg/kg, about 0.06 mg/kg to about 0.6 mg/kg, about 0.07 mg/kg to about 0.6 mg/kg, about 0.08 mg/kg to about 0.6 mg/kg, about 0.09 mg/kg to about 0.6 mg/kg, about 0.1 mg/kg to about 0.6 mg/kg, about 0.2 mg/kg to about 0.6 mg/kg, about 0.3 mg/kg to about 0.6 mg/kg, about 0.4 mg/kg to about 0.6 mg/kg, or about 0.5 mg/kg to about 0.6 mg/kg.

In some embodiments, the guide RNA and the mRNA are administered at the total amount of about 0.01 mg/kg to about 0.3 mg/kg, about 0.02 mg/kg to about 0.3 mg/kg, about 0.03 mg/kg to about 0.3 mg/kg, about 0.04 mg/kg to about 0.3 mg/kg, about 0.05 mg/kg to about 0.3 mg/kg, about 0.06 mg/kg to about 0.3 mg/kg, about 0.07 mg/kg to about 0.3 mg/kg, about 0.08 mg/kg to about 0.3 mg/kg, about 0.09 mg/kg to about 0.3 mg/kg, about 0.1 mg/kg to about 0.3 mg/kg, or about 0.2 mg/kg to about 0.3 mg/kg.

In some embodiments, the guide RNA and the mRNA are administered at the total amount of about 0.01 mg/kg, about 0.015 mg/kg, about 0.02 mg/kg, about 0.025 mg/kg, about 0.03 mg/kg, about 0.035 mg/kg, about 0.04 mg/kg, about 0.045 mg/kg, about 0.05 mg/kg, about 0.055 mg/kg, about 0.06 mg/kg, about 0.065 mg/kg, about 0.07 mg/kg, about 0.075 mg/kg, about 0.08 mg/kg, about 0.085 mg/kg, about 0.09 mg/kg, about 0.095 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.05 mg/kg, about 1.1 mg/kg, about 1.15 mg/kg, about 1.2 mg/kg, about 1.25 mg/kg, about 1.3 mg/kg, about 1.35 mg/kg, about 1.4 mg/kg, about 1.45 mg/kg, about 1.5 mg/kg, about 1.55 mg/kg, about 1.6 mg/kg, about 1.65 mg/kg, about 1.7 mg/kg, about 1.75 mg/kg, about 1.8 mg/kg, about 1.85 mg/kg, about 1.9 mg/kg, about 1.95 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5 mg/kg.

In some embodiments, the guide RNA and the mRNA are administered at a total amount of about 0.001 mg/kg to about 1000 mg/kg, about 0.002 mg/kg to about 950 mg/kg, about 0.003 mg/kg to about 900 mg/kg, about 0.004 mg/kg to about 850 mg/kg, about 0.005 mg/kg to about 800 mg/kg, about 0.006 mg/kg to about 750 mg/kg, about 0.007 mg/kg to about 700 mg/kg, about 0.008 mg/kg to about 650 mg/kg, about 0.009 mg/kg to about 600 mg/kg, about 0.01 mg/kg to about 500 mg/kg, about 0.02 mg/kg to about 500 mg/kg, about 0.03 mg/kg to about 450 mg/kg, about 0.04 mg/kg to about 400 mg/kg, about 0.05 mg/kg to about 350 mg/kg, about 0.06 mg/kg to about 300 mg/kg, about 0.07 mg/kg to about 250 mg/kg, about 0.08 mg/kg to about 200 mg/kg, about 0.09 mg/kg to about 150 mg/kg, about 0.1 mg/kg to about 100 mg/kg, about 0.2 mg/kg to about 90 mg/kg, about 0.3 mg/kg to about 85 mg/kg, about 0.4 mg/kg to about 80 mg/kg, about 0.5 mg/kg to about 75 mg/kg, about 0.6 mg/kg to about 70 mg/kg, about 0.7 mg/kg to about 65 mg/kg, about 0.8 mg/kg to about 60 mg/kg, about 0.9 mg/kg to about 55 mg/kg, about 1 mg/kg to about 50 mg/kg, about 2 mg/kg to about 45 mg/kg, about 3 mg/kg to about 40 mg/kg, about 4 mg/kg to about 35 mg/kg, about 5 mg/kg to about 30 mg/kg, about 6 mg/kg to about 25 mg/kg, about 7 mg/kg to about 20 mg/kg, about 8 mg/kg to about 15 mg/kg, or about 9 mg/kg to about 10 mg/kg.

In some embodiments, the guide RNA and the mRNA are administered at a total amount of about 0.005 mg/kg to about 1000 mg/kg, about 0.01 mg/kg to about 1000 mg/kg, about 0.05 mg/kg to about 1000 mg/kg, about 0.1 mg/kg to about 1000 mg/kg, about 0.5 mg/kg to about 1000 mg/kg, about 1 mg/kg to about 1000 mg/kg, about 5 mg/kg to about 1000 mg/kg, about 10 mg/kg to about 1000 mg/kg, about 20 mg/kg to about 1000 mg/kg, about 30 mg/kg to about 1000 mg/kg, about 40 mg/kg to about 1000 mg/kg, about 50 mg/kg to about 1000 mg/kg, about 60 mg/kg to about 1000 mg/kg, about 70 mg/kg to about 1000 mg/kg, about 80 mg/kg to about 1000 mg/kg, about 90 mg/kg to about 1000 mg/kg, about 100 mg/kg to about 1000 mg/kg, about 150 mg/kg to about 1000 mg/kg, about 200 mg/kg to about 1000 mg/kg, about 250 mg/kg to about 1000 mg/kg, about 300 mg/kg to about 1000 mg/kg, about 350 mg/kg to about 1000 mg/kg, about 400 mg/kg to about 1000 mg/kg, about 450 mg/kg to about 1000 mg/kg, about 500 mg/kg to about 1000 mg/kg, about 550 mg/kg to about 1000 mg/kg, about 600 mg/kg to about 1000 mg/kg, about 650 mg/kg to about 1000 mg/kg, about 700 mg/kg to about 1000 mg/kg, about 750 mg/kg to about 1000 mg/kg, about 800 mg/kg to about 1000 mg/kg, about 850 mg/kg to about 1000 mg/kg, about 900 mg/kg to about 1000 mg/kg, or about 950 mg/kg to about 1000 mg/kg.

In some embodiments, the guide RNA and the mRNA are administered at a total amount of about 0.001 mg/kg to about 950 mg/kg, about 0.001 mg/kg to about 900 mg/kg, about 0.001 mg/kg to about 850 mg/kg, about 0.001 mg/kg to about 800 mg/kg, about 0.001 mg/kg to about 750 mg/kg, about 0.001 mg/kg to about 700 mg/kg, about 0.001 mg/kg to about 650 mg/kg, about 0.001 mg/kg to about 600 mg/kg, about 0.001 mg/kg to about 550 mg/kg, about 0.001 mg/kg to about 500 mg/kg, about 0.001 mg/kg to about 450 mg/kg, about 0.001 mg/kg to about 400 mg/kg, about 0.001 mg/kg to about 350 mg/kg, about 0.001 mg/kg to about 300 mg/kg, about 0.001 mg/kg to about 250 mg/kg, about 0.001 mg/kg to about 200 mg/kg, about 0.001 mg/kg to about 150 mg/kg, about 0.001 mg/kg to about 100 mg/kg, about 0.001 mg/kg to about 90 mg/kg, about 0.001 mg/kg to about 80 mg/kg, about 0.001 mg/kg to about 70 mg/kg, about 0.001 mg/kg to about 60 mg/kg, about 0.001 mg/kg to about 50 mg/kg, about 0.001 mg/kg to about 40 mg/kg, about 0.001 mg/kg to about 30 mg/kg, about 0.001 mg/kg to about 20 mg/kg, about 0.001 mg/kg to about 10 mg/kg, about 0.001 mg/kg to about 9 mg/kg, about 0.001 mg/kg to about 8 mg/kg, about 0.001 mg/kg to about 7 mg/kg, about 0.001 mg/kg to about 6 mg/kg, about 0.001 mg/kg to about 5 mg/kg, about 0.001 mg/kg to about 4 mg/kg, about 0.001 mg/kg to about 3 mg/kg, about 0.001 mg/kg to about 2 mg/kg, about 0.001 mg/kg to about 1 mg/kg, about 0.001 mg/kg to about 0.9 mg/kg, about 0.001 mg/kg to about 0.8 mg/kg, about 0.001 mg/kg to about 0.7 mg/kg, about 0.001 mg/kg to about 0.6 mg/kg, about 0.001 mg/kg to about 0.5 mg/kg, about 0.001 mg/kg to about 0.4 mg/kg, about 0.001 mg/kg to about 0.3 mg/kg, about 0.001 mg/kg to about 0.2 mg/kg, about 0.001 mg/kg to about 0.1 mg/kg, about 0.001 mg/kg to about 0.09 mg/kg, about 0.001 mg/kg to about 0.08 mg/kg, about 0.001 mg/kg to about 0.07 mg/kg, about 0.001 mg/kg to about 0.06 mg/kg, about 0.001 mg/kg to about 0.05 mg/kg, about 0.001 mg/kg to about 0.04 mg/kg, about 0.001 mg/kg to about 0.03 mg/kg, about 0.001 mg/kg to about 0.02 mg/kg, about 0.001 mg/kg to about 0.01 mg/kg, about 0.001 mg/kg to about 0.009 mg/kg, about 0.001 mg/kg to about 0.008 mg/kg, about 0.001 mg/kg to about 0.007 mg/kg, about 0.001 mg/kg to about 0.006 mg/kg, about 0.001 mg/kg to about 0.005 mg/kg, about 0.001 mg/kg to about 0.004 mg/kg, about 0.001 mg/kg to about 0.003 mg/kg, or about 0.001 mg/kg to about 0.002 mg/kg.

### Lipid Nanoparticle (LNP)

The LNPs described herein can be designed for one or more specific applications or targets. The elements of a nanoparticle (LNP) composition or a composition can be selected based on a particular application or target, and/or based on the efficacy, toxicity, expense, ease of use, and availability. Similarly, the particular formulation of a nanoparticle composition is a composition comprising one or more described lipids. may be selected for the particular application or target. Suitable phosphate charge neutralizers to be used in formulations include, but are not limited to, Spermidine and 1,3-propanediamine.

In some embodiments, LNPs are prepared in accordance with the methods described in Conway, A. et al. 2019 Mol. Ther. 27, 866-877, and Villiger, L. et al 2021 Nat. Biomed. Eng. 5, 179-18, which are incorporated by reference. In some embodiments, LNPs are composed of an amino lipid, a monomethoxypolyethylene glycol (or methoxypoluyethyle glycol) of average molecular weight 2000 Da conjugated to a lipid called PEG-Lipid, cholesterol and 1,2-distearoyl-sn-glycero-3-phosphocholine (DSPC). In some embodiments, LNPs are composed of proprietary ionizable cationic lipid, 1,2-distearoyl-sn-glycero-3-phosphocholine, cholesterol, an ionizable amino lipid, and a PEG-lipid.

In some embodiments, LNPs have an average hydrodynamic diameter of about 30-about 160, about 35-about 160, about 40-about 160, about 45-about 160, about 50-about 160, about 55-about 160, about 60-about 160, about 65-about 160, about 70-about 160, about 75 about 160, about 80-about 160, about 85-about 160, about 90-about 160, about 95-about 160, about 100-about 160, about 105-about 160, about 110-about 160, about 115-about 160, about 120-about 160, about 125-about 160, about 130-about 160, about 135-about 160, about 140-about 160, about 145-about 160, about 150-about 160, about 30-about 155, about 30-about 150, about 30-about 145, about 30-about 140, about 30-about 135, about 30-about 130, about 30-about 125, about 30-about 120, about 30-about 115, about 30-about 110, about 30-about 105, about 30-about 100, about 30-about 95, about 30-about 90, about 30-about 85, about 30-about 80, about 30-about 75, about 30-about 70, about 30-about 65, about 30-about 60, about 30-about 55, about 30-about 50, about 30-about 45, about 30-about 40, about 30-about 45, about 35-about 45, about 35-about 50, about 40-about 50, about 40-about 55, about 45-about 55, about 45-about 60, about 50-about 60, about 50-about 65, about 55-about 60, about 55-about 65, about 55-about 70, about 60-about 70, about 60-about 75, about 65-about 75, about 65-about 80, about 70-about 80, about 70-about 85, about 75-about 85, about 75-about 90, about 80-about 90, about 80-about 95, about 85-about 95, about 85-about 100, about 90-about 100, about 90-about 105, about 95-about 105, about 95-about 110, about 100-about 110, about 100-about 115, about 105-about 115, about 105-about 120, about 110-about 120, about 110-about 125, about 115-about 125, about 115-about 130, about 120-about 130, about 120-about 135, about 125-about 135, about 125-about 140, about 130-about 140, about 130-about 145, about 35-about 140, about 45-about 130, about 55-about 120, about 65-about 110, about 75-about 100, or about 85-about 90 nm.

In some embodiments, LNPs comprise about 1-about 97, about 5-about 97, about 10-about 97, about 15-about 97, about 20-about 97, about 25-about 97, about 30-about 97, about 35-about 97, about 40-about 97, about 45-about 97, about 50-about 97, about 55-about 97, about 60-about 97, about 65-about 97, about 70-about 97, about 75-about 97, about 80-about 97, about 1-about 95, about 1-about 90, about 1-about 85, about 1-about 80, about 1-about 75, about 1-about 70, about 1-about 65, about 1-about 60, about 1-about 55, about 1-about 50, about 1-about 45, about 1-about 40, about 1-about 35, about 1-about 30, about 1-about 25, about 1-about 20, about 1-about 15, about 1-about 10, about 10-about 30, about 10-about 35, about 15-about 35, about 15-about 40, about 20-about 40, about 20-about 45, about 25-about 45, about 25-about 50, about 30-about 50, about 30-about 55, about 35-about 55, about 35-about 60, about 40-about 60, about 40-about 65, about 45-about 65, about 45-about 70, about 50-about 70, about 50-about 75, about 55-about 75, about 55-about 80, or about 60-about 80% of amino lipids (in mol %).

In some embodiments, LNPs comprise about 1-about 40, about 1-about 38, about 1-about 36, about 1-about 34, about 1-about 32, about 1-about 30, about 1-about 28, about 1-about 26, about 1-about 24, about 1-about 22, about 1-about 20, about 1-about 18, about 1-about 16, about 1-about 14, about 1-about 12, about 1-about 10, about 1-about 8, about 1-about 6, about 1-about 4, about 1-about 2, about 2-about 40, about 4-about 40, about 6-about 40, about 8-about 40, about 10-about 40, about 12-about 40, about 14-about 40, about 16-about 40, about 18-about 40, about 20-about 40, about 22-about 40, about 24-about 40, about 26-about 40, about 28-about 40, about 30-about 40, about 32-about 40, about 34-about 40, about 36-about 40, about 38-about 40, about 2-about 35, about 2-about 30, about 2 about 25, about 2-about 20, about 2-about 15, about 2-about 10, about 2-about 8, about 2-about 6, or about 2-about 4% DSPC (in mol %).

In some embodiments, LNPs comprise about 1-about 20, about 1-about 19, about 1-about 18, about 1-about 17, about 1-about 16, about 1-about 15, about 1-about 14, about 1-about 13, about 1-about 12, about 1-about 11, about 1-about 10, about 1-about 9, about 1-about 8, about 1-about 7, about 1-about 6, about 1-about 5, about 1-about 4, about 1-about 3, about 2-about 6, about 3-about 7, about 4-about 8, about 5-about 9, about 6-about 10, about 7-about 11, about 8-about 12, about 9-about 13, about 10-about 14, about 11-about 15, about 12-about 16, about 13-about 17, about 14-about 18, about 15-about 19, or about 16-about 20% PEG-Lipid (in mol %).

In some embodiments, LNPs comprise about 1-about 97, about 5-about 97, about 10-about 97, about 15-about 97, about 20-about 97, about 25-about 97, about 30-about 97, about 35-about 97, about 40-about 97, about 45-about 97, about 50-about 97, about 55-about 97, about 60-about 97, about 65-about 97, about 70-about 97, about 75-about 97, about 80-about 97, about 1-about 95, about 1-about 90, about 1-about 85, about 1-about 80, about 1-about 75, about 1-about 70, about 1-about 65, about 1-about 60, about 1-about 55, about 1-about 50, about 1-about 45, about 1-about 40, about 1-about 35, about 1-about 30, about 1-about 25, about 1-about 20, about 1-about 15, about 1-about 10, about 10-about 30, about 10-about 35, about 15-about 35, about 15-about 40, about 20-about 40, about 20-about 45, about 25-about 45, about 25-about 50, about 30-about 50, about 30-about 55, about 35-about 55, about 35-about 60, about 40-about 60, about 40-about 65, about 45-about 65, about 45-about 70, about 50-about 70, about 50-about 75, about 55-about 75, about 55-about 80, or about 60-about 80% of cholesterol (in mol %).

In some embodiments, LNPs comprise about 1-about 97, 5-about 97, 10-about 97, 15 about 97, 20-about 97, 25-about 97, 30-about 97, 35-about 97, 40-about 97, 45-about 97, 50-about 97, 55-about 97, 60-about 97, 65-about 97, 70-about 97, 75-about 97, 80-about 97, 1-about 95, 1-about 90, 1-about 85, 1-about 80, 1-about 75, 1-about 70, 1-about 65, 1-about 60, 1-about 55, 1-about 50, 1-about 45, 1-about 40, 1-about 35, 1-about 30, 1-about 25, 1-about 20, 1-about 15, 1-about 10, 10-about 30, 10-about 35, 15-about 35, 15-about 40, 20-about 40, 20-about 45, 25-about 45, 25-about 50, 30-about 50, 30-about 55, 35-about 55, 35-about 60, 40-about 60, 40-about 65, 45-about 65, 45-about 70, 50-about 70, 50-about 75, 55-about 75, 55-about 80, or 60-about 80% of amino lipids; 1-about 40, 1-about 38, 1-about 36, 1-about 34, 1-about 32, 1-about 30, 1-about 28, 1-about 26, 1-about 24, 1-about 22, 1-about 20, 1-about 18, 1-about 16, 1-about 14, 1-about 12, 1-about 10, 1-about 8, 1-about 6, 1-about 4, 1-about 2, 2-about 40, 4-about 40, 6-about 40, 8-about 40, 10-about 40, 12-about 40, 14-about 40, 16-about 40, 18-about 40, 20-about 40, 22-about 40, 24-about 40, 26-about 40, 28-about 40, 30-about 40, 32-about 40, 34-about 40, 36-about 40, 38-about 40, 2-about 35, 2-about 30, 2-about 25, 2-about 20, 2-about 15, 2-about 10, 2-about 8, 2-about 6, or 2-about 4% DSPC; 1-about 20, 1-about 19, 1-about 18, 1-about 17, 1-about 16, 1-about 15, 1-about 14, 1-about 13, 1-about 12, 1-about 11, 1-about 10, 1-about 9, 1-about 8, 1-about 7, 1-about 6, 1-about 5, 1 about 4, 1-about 3, 2-about 6, 3-about 7, 4-about 8, 5-about 9, 6-about 10, 7-about 11, 8-about 12, 9-about 13, 10-about 14, 11-about 15, 12-about 16, 13-about 17, 14-about 18, 15-about 19, and about 16-about 20% PEG-Lipid, with the balance being cholesterol (all in mol %).

### Amino Lipid

Described herein are LNP compositions comprising an amino lipid, a phospholipid, a PEGlipid, a cholesterol or a derivative thereof, a payload, or any combination thereof. In some embodiments, the LNP composition comprises an amino lipid. In one aspect, disclosed herein is an amino lipid having the structure of. Formula (I), or a pharmaceutically acceptable salt or solvate thereof,

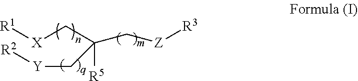

wherein
each of R¹ and R² is independently C₇-C₂₂ alkyl, C₇-C₂₂ alkenyl, C₃-C₈ cycloalkyl, —C₂-C₁₀ alkylene-L-R⁶, or

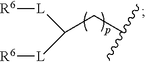

wherein each of the alkyl, alkylene, alkenyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—O, S, or a bond;
each of L is independently —C(═O)NR⁴—, —NR⁴C(═O)—, —C(═O)O—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl,
wherein the alkylene, heterocycloalkyl and heterocycloaryl is independently substituted or unsubstituted; each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
p is an integer selected from 1 to 10; and
each of n, m, and q is independently 0, 1, 2, 3, 4, or 5.

In some embodiments of Formula (I), if the structure carries more than one asymmetric C-atom, each asymmetric C-atom independently represents racemic, chirally pure R and/or chirally pure S isomer, or a combination thereof.

In some embodiments, each of n, in, and q in Formula (I) is independently 0, 1, 2, or 3. In some embodiments, each of n, m, and q in Formula (I) is 1.

In some embodiments, the compound of Formula (1) has a structure of Formula (Ia), or a pharmaceutically acceptable salt or pharmaceutically acceptable solvate thereof:

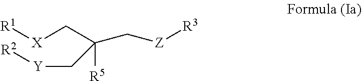

wherein
each of R¹ and R² is independently C₇-C₂₂ alkyl, C₇-C₂₂ alkenyl, C₃-C₈ cycloalkyl, —C₂-C₁₀ alkylene-L-R⁶, or

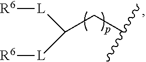

wherein each of the alkyl, alkylene, alkenyl, and cycloalkyl is independently substituted or unsubstituted;
each of X, Y, and Z is independently C(═O)NR⁴—, —NR⁴C(D)-, —C(═O)O—, —OC(═O)—, —OC(═O)O—, —NR⁴C(═O)O—, —OC(═O)NR⁴—, —NR⁴C═O)NR⁴—, —NR⁴C(═NR⁴)NR⁴—, —C(═S)NR⁴—, —NR⁴C(═S)—, —C(E)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene —OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocyclowyl, wherein the alkylene, heterocycloalkyl and heterocycloaryl is independently substituted or unsubstituted;
each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
p is an integer selected from 1 to 10.

In some embodiments of Formula (Ia), if the structure carries more than one asymmetric Catom, each asymmetric C-atom independently represents racemic, chirally pure R and/or chirally pure S isomer, or a combination thereof.

In some embodiments, R¹ and R² in Formula (I) and Formula (Ia) is independently C₇-C₂₂ alkyl, C₇-C₂₂ alkenyl, —C₂-C₁₀ alkylene-L-R⁶, or

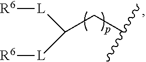

wherein each of the alkyl, alkylene, alkenyl, and cycloalkyl is independently substituted or unsubstituted. In some embodiments, R¹ and R² in Formula (I) and Formula (Ia) is independently C₁₀-C₂₀ alkyl, C₁₀-C₂₀ alkenyl, —C₈-C₇ alkylene-L-R⁶, or

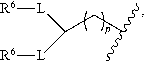

wherein each of the alkyl, alkylene, alkenyl, and cycloalkyl is independently substituted or unsubstituted. In some embodiments, R¹ in Formula (I) and Formula (Ia) is

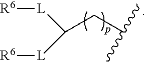

In some embodiments, each of L in Formula (I) and Formula (Ia) is independently O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene.

In some embodiments, each of R⁶ in Formula (I) and Formula (Ia) is independently substituted or unsubstituted linear C₃-C₂₂ alkyl or substituted or unsubstituted linear C₃-C₂₂ alkenyl. In some embodiments, each of R⁶ in Formula (I) and Formula (Ia) is independently substituted or unsubstituted C₃-C₂₀ alkyl or substituted or unsubstituted C₃-C₂₀ alkenyl. In some embodiments, each of R⁶ in Formula (I) and Formula (Ia) is independently substituted or unsubstituted C₃-C₁₀ alkyl or substituted or unsubstituted C₃-C₁₀ alkenyl. In some embodiments, each of R⁶ in Formula (I) and Formula (Ia) is independently substituted or unsubstituted C₃-C₁₀ alkyl. In some embodiments, each of R⁶ in Formula (I) and Formula (Ia) is independently substituted or unsubstituted linear C₃-C₁₀ alkyl. In some embodiments, each of R⁶ in Formula (I) and Formula (Ia) is independently substituted or unsubstituted n-pentyl, n-hexyl, n-heptyl, n-octyl, n-nonyl, n-decyl, n-undecyl, or n-dodecyl. In some embodiments, each of R⁶ in Formula (I) and Formula (Ia) is independently substituted or unsubstituted n-octyl. In some embodiments, each of R⁶ in Formula (I) and Formula (Ia) is n-octyl.

In some embodiments, each of L in Formula (I) and Formula (Ia) is independently —C(═O)O—, —OC(═O)—, —C₁-C₁₀ alkylene-O—, or O. In some embodiments, each of L in Formula (I) and Formula (Ia) is O. In some embodiments, each of L in Formula (I) and Formula (Ia) is —C₁-C₃ alkylene-O—. In some embodiments, p in Formula (I) and Formula (Ia) is 1, 2, 3, 4, or 5. In some embodiments, p in Formula (I) and Formula (Ia) is 2.

In some embodiments, R¹ in Formula (I) and Formula (Ia) is

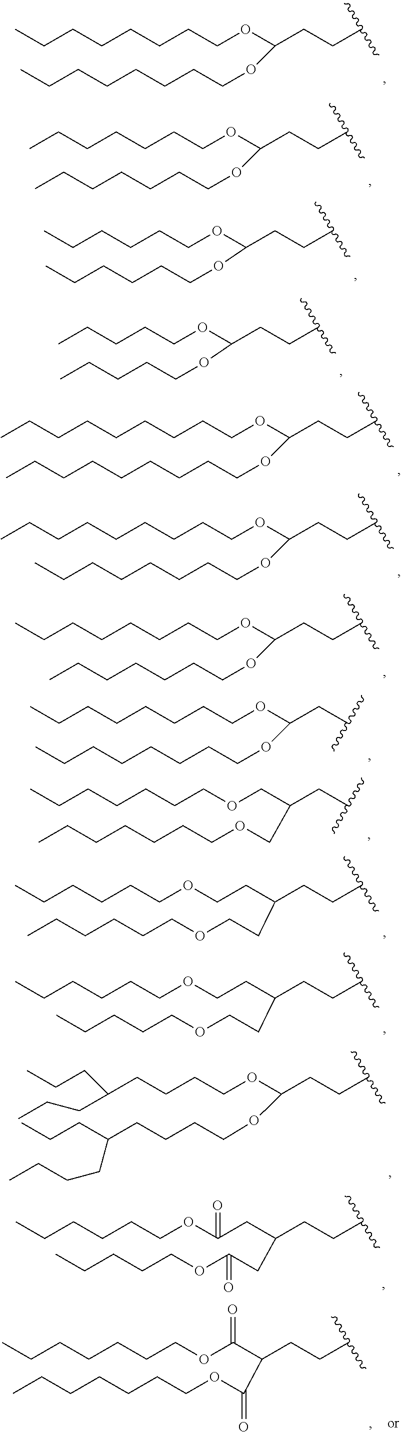

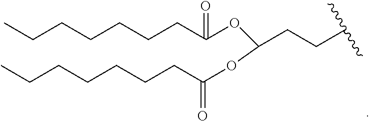 [SMILES: *CCC(OC(=O)CCCCCCC)OC(=O)CCCCCCC]

In some embodiments each of R⁴ in Formula (I) and Formula (Ia) is independently H or substituted or unsubstituted C₁-C₄ alkyl. In some embodiments, each of R⁴ in Formula (I) and Formula (Ia) is independently substituted or unsubstituted linear C₁-C₄ alkyl. In some embodiments, each of R⁴ in Formula (1) and Formula (Ia) is H. In some embodiments, each of R⁴ in Formula (I) and Formula (Ia) is independently H, —CH₃, —CH₂CH₃, —CH₂CH₂CH₃, or —CH(CH₃)₂. In some embodiments, each of R⁴ in Formula (I) and Formula (Ia) is independently H or —CH₃. In some embodiments, each of R⁴ in Formula (1) and Formula (Ia) is —CH₃.

In some embodiments, X in Formula (I) and Formula (Ia) is —C(═O)O— or —OC(═O))—. In some embodiments, X in Formula (I) and Formula (Ia) is —C(═O)NR⁴— or —NR⁴C(═O)—. In some embodiments, X in Formula (I) and Formula (Ia) is —C(═O)N(CH₃)—, —N(CH₃)C(═O)—, —C(═O)NH—, or —NHC(═O)—. In some embodiments, X in Formula (I) and Formula (Ia) is —C(═O))NH—, —C(═O)N(CH₃)—, —OC(═O))—, —NHC(═O)—, —N(CH₃)C(═O))—, —C(═O)O—, —OC(═O)O—, —NHC(═O)O—, —N(CH₃)C(═O)O—, —OC(═O))NH—, —OC(═O)N(CH₃)—, —NHC(═O)NH—, —N(CH₃)C(═O))NH—, —NHC(═O)N(CH₃)—, —N(CH₃)C(═O)N(CH₃)—, NHC(═NH)NH—, —N(CH₃)C(═NH)NH—, —NHC(═NH)N(CH₃)—, —N(CH₃)C(═NH)N(CH₃)—, NHC(═NMe)NH—, —N(CH₃)C(═NMe)NH—, —NHC(═NMe)N(CH₃)—, or —N(CH₃)C(═NMe)N(CH₃)—.

In some embodiments. R² in Formula (I) and Formula (Ia) is C₇-C₂₂ alkyl, C₇-C₂₂ alkenyl, —C₂-C₁₀ alkylene-L-R⁶,

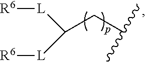

wherein each of the alkyl, alkylene, alkenyl, and cycloalkyl is independently substituted or unsubstituted. In some embodiments, R² in Formula (I) and Formula (Ia) is substituted or unsubstituted C₇-C₂₂ alkyl or substituted or unsubstituted C₇-C₂₂ alkenyl. In some embodiments, R² in Formula (I) and Formula (Ia) is substituted or unsubstituted linear C₇-C₂₂ alkyl or substituted or unsubstituted linear C₇-C₂₂ alkenyl. In some embodiments, R² in Formula (I) and Formula (Ia) is substituted or unsubstituted C₁₀-C₂₀ alkyl or substituted or unsubstituted C₁₀-C₂₀ alkenyl. In some embodiments, R² in Formula (I) and Formula (Ia) is unsubstituted C₁₀-C₂₀ alkyl. In some embodiments, R² in Formula (I) and Formula (Ia) is unsubstituted C₁₀-C₂₀ alkenyl. In some embodiments, R² in Formula (I) and Formula (Ia) is —C₂-C₁₀ alkylene-L-R⁶. In some embodiments, R² in Formula (I) and Formula (Ia) is —C₂-C₁₀ alkylene-C(═O)O— R⁶ or —C₂-C₁₀ alkylene-OC(═O)—R⁶.

In some embodiments, R² in Formula (I) and Formula (Ia) is

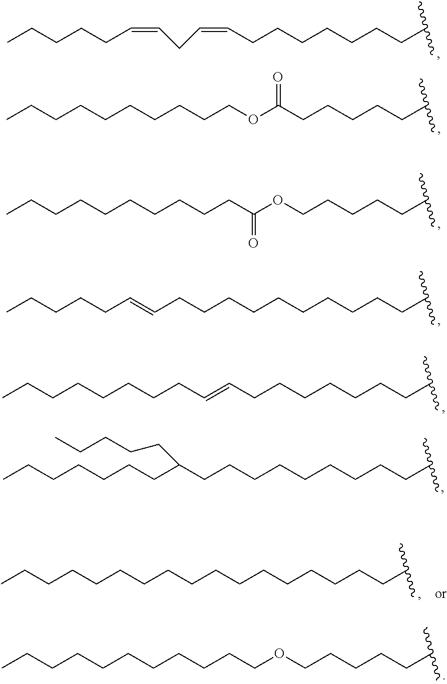

In some embodiments, Y in Formula (I) and Formula (Ia) is —C(═O)O— or —OC(O)—. In some embodiments, Y in Formula (I) and Formula (Ia) is —C(═O)NR⁴— or —NR⁴C(═O)—. In some embodiments, Y in Formula (I) and Formula (Ia) is —C(═O)N(CH₃)—, —N(CH₃)C(═O)—, —C(═O)NH—, or —NHC(═O)—. In some embodiments, Y in Formula (I) and Formula (Ia) is —OC(═O)O—, —NR⁴C(═O)O—, —OC(═O)NR⁴—, or —NR⁴C(═O)NR⁴—. In some embodiments. Y in Formula (I) and Formula (Ia) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)— or —N(CH₃)C(═O)NH—. In some embodiments, Y in Formula (I) and Formula (Ia) is —OC(═O)O—, —NHC(═O)O—, —OC(═O)NH—, or —NHC(═O)NH—.

In some embodiments, R³ in Formula (I) and Formula (Ia) is —C₀-C₁₀ alkylene-NR⁷R⁸ or —C₀-C₁₀ alkylene-heterocycloalkyl, wherein the alkylene and heterocycloalkyl is independently substituted or unsubstituted. In some embodiments, R³ in Formula (I) and Formula (Ta) is —C₀-C₁₀ alkylene-NR⁷R⁸. In some embodiments, R³ in Formula (I) and Formula (Ia) is —C₁-C₆ alkylene-NR⁷R⁸. In some embodiments, R³ in Formula (I) and Formula (Ia) is —C₁-C₄ alkylene-NR⁷R⁸. In some embodiments, R³ in Formula (I) and Formula (Ia) is —C₁— alkylene-NR⁷R⁸. In some embodiments, R³ in Formula (I) and Formula (Ia) is —C₂-alkylene-NR⁷R⁸. In some embodiments, R³ in Formula (I) and Formula (Ia) is —C₃— alkylene-NR⁷R⁸. In some embodiments, R³ in Formula (I) and Formula (Ia) is —C₄— alkylene-NR⁷R⁸. In some embodiments, R³ in Formula (I) and Formula (Ia) is —C₅— alkylene-NR⁷R⁸. In some embodiments, R³ in Formula (I) and Formula (Ia) is —C₀-C₁₀ alkylene-heterocycloalkyl. In some embodiments, R³ in Formula (I) and Formula (Ia) is —C₁-C₆ alkylene-heterocycloalkyl, wherein the heterocycloalkyl comprises 1 to 3 nitrogen and 0-2 oxygen. In some embodiments, R³ in Formula (I) and Formula (Ia) is —C₁-C₆ alkylene-heterocycloaryl.

In some embodiments, each of R⁷ and R⁸ in Formula (I) and Formula (Ia) is independently hydrogen or substituted or unsubstituted C₁-C₆ alkyl. In some embodiments, each of R⁷ and R⁸ is independently hydrogen or substituted or unsubstituted C₁-C₃ alkyl. In some embodiments, each of R⁷ and R⁸ is independently substituted or unsubstituted C₁-C₃ alkyl. In some embodiments, each of R⁷ and R⁸ is independently —CH₃, —CH₂CH₃, —CH₂CH₂CH₃, or —CH(CH₃)₂. In some embodiments, each of R and R⁸ is CH₃. In some embodiments, each of R⁷ and R⁸ is —CH₂CH₃.

In some embodiments, R⁷ and R⁸ in Formula (I) and Formula (Ia) taken together with the nitrogen to which they are attached form a substituted or unsubstituted C₂-C₆ heterocyclyl. In some embodiments, R⁷ and R⁸ taken together with the nitrogen to which they are attached form a substituted or unsubstituted C₂-C₆ heterocycloalkyl. In some embodiments, R⁷ and R⁸ taken together with the nitrogen to which they are attached form a substituted or unsubstituted 3-7 membered heterocycloalkyl.

In some embodiments, R³ in Formula (I) and Formula (Ia) is

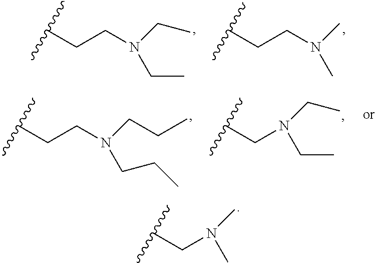

In some embodiments. R³ in Formula (I) and Formula (Ia) is

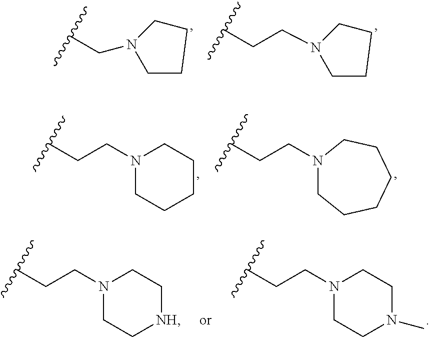

In some embodiments. R³ in Formula (I) and Formula (Ia) is

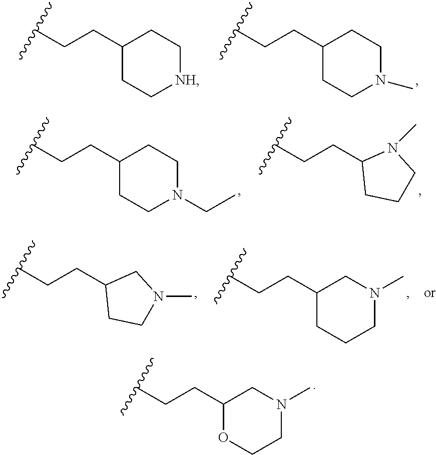

In some embodiments, Z in Formula (I) and Formula (Ia) is —C(═O)O— or —OC(═O)—. In some embodiments, Z in Formula (I) and Formula (Ia) is —C(═O)NR₄— or —NR₄C(═O)—. In some embodiments, Z in Formula (I) and Formula (Ia) is —C(═O)N(CH₃)—, —N(CH₃)C(═O)—, —C(═O)NH—, or —NHC(═O)—. In some embodiments, Z in Formula (I) and Formula (Ia) is —OC(═O)O—, —NR⁴C(═O)O—, —OC(O)NR⁴—, or —NR⁴C(═O)NR⁴—. In some embodiments. Z in Formula (I) and Formula (Ia) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)—, —NHC(═O)N(CH₃)— or —N(CH₃)C(═O)NH—. In some embodiments, Y in Formula (I) and Formula (Ia) is —OC(═O)O—, —NHC(═O)O—, —OC(═O)NH—, or —NHC(═O)NH—.

In some embodiments, R⁵ in Formula (I) and Formula (Ia) is hydrogen or substituted or unsubstituted C₁-C₃ alkyl. In some embodiments, R⁵ in Formula (I) and Formula (Ia) is H, —CH₃, —CH—)CH₃, —CH₂CH₂CH₃, or —CH(CH₃)₂. In some embodiments, R⁵ in Formula (I) and Formula (Ia) is H.

In some embodiments, the LNP comprises a plurality of amino lipids. For example, the LNP composition can comprise 2, 3, 4, 5, 6.7, 8, 9. 10, or more amino lipids. For another example, the LNP composition can comprise at least 2, at least 3, at least 4, at least 5, at least 6, at least 7, at least 9, at least 10, or at least 20 amino lipids. For yet another example, the LNP composition can comprise at most 2, at most 3, at most 4, at most 5, at most 6, at most 7, at most 9, at most 10, at most 20, or at most 30 amino lipids.

In some embodiments, the LNP composition comprises a first amino lipid. In some embodiments, the LNP composition comprises a first amino lipid and a second amino lipid. In some embodiments, the LNP composition comprises a first amino lipid, a second amino lipid, and a third amino lipid. In some embodiments, the LNP composition comprises a first amino lipid, a second amino lipid, a third amino lipid, and a fourth amino lipid. In some embodiments, the LNP composition does not comprise a fourth amino lipid. In some embodiments, the LNP composition does not comprise a third amino lipid. In some embodiments, a molar ratio of the first amino lipid to the second amino lipid is from about 0.1 to about 10. In some embodiments, a molar ratio of the first amino lipid to the second amino lipid is from about 0.20 to about 5. In some embodiments, a molar ratio of the first amino lipid to the second amino lipid is from about 0.25 to about 4. In some embodiments, a molar ratio of the first amino lipid to the second amino lipid is about 0.25, about 0.33, about 0.5, about 1, about 2, about 3, or about 4.

In some embodiments, a molar ratio of the first amino lipid: the second amino lipid: the third amino lipid is about 4:1:1. In some embodiments, a molar ratio of the first amino lipid: the second amino lipid: the third amino lipid is about 1:1:1. In some embodiments, a molar ratio of the first amino lipid: the second amino lipid: the third amino lipid is about 2:1:1. In some embodiments, a molar ratio of the first amino lipid: the second amino lipid: the third amino lipid is about 2:2:1. In some embodiments, a molar ratio of the first amino lipid: the second amino lipid: the third amino lipid is about 3:2:1. In some embodiments, a molar ratio of the first amino lipid: the second amino lipid: the third amino lipid is about 3:1:1. In some embodiments, a molar ratio of the first amino lipid: the second amino lipid: the third amino lipid is about 5:1:1. In some embodiments, a molar ratio of the first amino lipid: the second amino lipid: the third amino lipid is about 3:3:1. In some embodiments, a molar ratio of the first amino lipid: the second amino lipid: the third amino lipid is about 4:4:1.

In some embodiments, the LNP composition comprises one or more amino lipids. In some embodiments, the one or more amino lipids comprise from about 40 mol % to about 65 mol % of the total lipid present in the particle. In some embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle. In some embodiments, the first amino lipid comprises from about 1 mol % to about 99 mol % of the total amino lipids present in the particle. In some embodiments, the first amino lipid comprises from about 16.7 mol % to about 66.7 mol % of the total amino lipids present in the particle. In some embodiments, the first amino lipid comprises from about 20 mol % to about 60 mol % of the total amino lipids present in the particle.

In some embodiments, the amino lipid is an ionizable lipid. An ionizable lipid can comprise one or more ionizable nitrogen atoms. In some embodiments, at least one of the one or more ionizable nitrogen atoms is positively charged. In some embodiments, at least 10 mol %, 20 mol %, 30 mol %, 40 mol %, 50 mol %, 60 mol %, 70 mol %, 80 mol %. 90 mol %, 95 mol %, or 99 mol % of the ionizable nitrogen atoms in the LNP composition are positively charged. In some embodiments, the amino lipid comprises a primary amine, a secondary amine, a tertiary amine, an imine, an amide, a guanidine moiety, a histidine residue, a lysine residue, an arginine residue, or any combination thereof. In some embodiments, the amino lipid comprises a primary amine, a secondary amine, a tertiary amine, a guanidine moiety, or any combination thereof. In some embodiments, the amino lipid comprises a tertiary amine.

In some embodiments, the amino lipid is a cationic lipid. In some embodiments, the amino lipid is an ionizable lipid. In some embodiments, the amino lipid comprises one or more nitrogen atoms. In some embodiments, the amino lipid comprises one or more ionizable nitrogen atoms. Exemplary cationic and/or ionizable lipids include, but are not limited to, 3-(didodecylamino)-N1,N1,4-tri dodecyl-1-piperazineethan amine (KL10), N142-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC 3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethy 1-3-[(9Z,12Z)-octadeca-9,12-dien-1-yloxy]propan-1-amine (Octyl-CLinDMA (2S)).

In some embodiments, an amino lipid described herein can take the form of a salt, such as a pharmaceutically acceptable salt. All pharmaceutically acceptable salts of the amino lipid are encompassed by this disclosure. As used herein, amino lipid also includes its pharmaceutically acceptable salts, and its diastereomeric, enantiomeric, and epimeric forms.

In some embodiments, an amino lipid described herein, possesses one or more stereocenters and each stereocenter exists independently in either the R or S configuration. The lipids presented herein include all diastereomeric, enantiomeric, and epimeric forms as well as the appropriate mixtures thereof. The lipids provided herein include all cis. trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In one aspect, stereoisomers are obtained by stereoselective synthesis.

In some embodiments, the lipids such as the amino lipids are substituted based on the structures disclosed herein. In some embodiments, the lipids such as the amino lipids are unsubstituted. In another embodiment, the lipids described herein are labeled isotopically (e.g. with a radioisotope) or by another other means, including, but not limited to, the use of chromophores or fluorescent moieties, bioluminescent labels, or chemiluminescent labels.

Lipids described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S ¹⁸F, ³⁶Cl. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the asymmetric carbon atom of the amino lipid is present in enantiomerically enriched form. In certain embodiments, the asymmetric carbon atom of the amino lipid has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (S)- or (R)-configuration.

In some embodiments, the disclosed amino lipids can be converted to N-oxides. In some embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as N O or N*—O—. In some embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown and claimed nitrogen containing compounds are also considered. Accordingly, also disclosed herein are N-hydroxy and Nal koxy (e.g., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives of the described amino lipids.

### PEG-Lipid

As used herein, a “PEG lipid” or “PEG-lipid” refers to a lipid comprising a polyethylene glycol component.

In some embodiments, the described LNP composition comprises a PEG-lipid. In some embodiments, the described LNP composition comprises two or more PEG-lipids. Exemplary PEG-lipids include, but are not limited to, the lipids. Exemplary PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof. In some embodiments, PEG moiety is an optionally substituted linear or branched polymer of ethylene glycol or ethylene oxide. In some embodiments, the PEG moiety is substituted, e.g., by one or more alkyl, alkoxy, acyl, hydroxy, or aryl groups. In some embodiments, the PEG moiety includes PEG copolymer such as PEG-polyurethane or PEG-polypropylene (see, e.g., j. Milton Harris, Poly(ethylene glycol) chemistry: biotechnical and biomedical applications (1992)). In some embodiments, the PEG moiety does not include PEG copolymers, e.g., it may be a PEG monopolymer. Exemplary PEG-lipids include, but are not limited to, PEG-dilauroylglycerol, PEG-dimyristoylglycerol (PEG-DMG), PEG-dipalmitoylglycerol, PEG-distearoylgiycerol (PEG-DSPE), PEG-dipalmitoylglycerol, PEG-disteiylglycerol, PEG-dilawylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)−2000]).

In some embodiments, a PEG-lipid is a PEG-lipid conjugate, for example, PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g. POZ-DAA conjugates; see, e.g., WO 2010/006282), polyamide oligomers (e.g., ATTA-lipid conjugates), and mixtures thereof.

A PEG-lipid can comprise one or more ethylene glycol units, for example, at least 1, at least 2, at least 5, at least 10, at least 20, at least 30, at least 40, at least 50, at least 60, at least 70, at least 80, at least 90, at least 100, at least 120, or at least 150 ethylene glycol units. In some embodiments, a number average molecular weight of the PEG-lipids is from about 200 Da to about 5000 Da. In some embodiments, a number average molecular weight of the PEG-lipids is from about 500 Da to about 3000 Da. In some embodiments, a number average molecular weight of the PEG-lipids is from about 750 Da to about 2500 Da. In some embodiments, a number average molecular weight of the PEG-lipids is from about 750 Da to about 2500 Da. In some embodiments, a number average molecular weight of the PEG-lipids is about 500 Da, about 750 Da, about 1000 Da, about 1250 Da, about 1500 Da, about 1750 Da, or about 2000 Da. In some embodiments, a polydispersity index (PD1) of the one or more PEG-lipids is smaller than 2. In some embodiments, a PDI of the one or more PEG-lipids is at most 1.1, 1.2, 1.3, 1.4, 1.5, 1.6, 1.7, 1.8, 1.9, 2.0, 2.1, 2.2, 2.3. 2.4, 2.5, 2.6, 2.7, 2.8, 2.9, or 3.0. In some embodiments, a PDI of the one or more PEG-lipids is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0.

In some embodiments, the PEG-lipid comprises from about 0.1 mol % to about 10 mol % of the total lipid present in the particle. In some embodiments, the PEG-lipid comprises from about 0.1 mol % to about 6 mol % of the total lipid present in the particle. In some embodiments, the PEG-lipid comprises from about 0.5 mol % to about 5 mol % of the total lipid present in the particle. In some embodiments, the PEG-lipid comprises from about 1 mol % to about 3 mol % of the total lipid present in the particle. In some embodiments, the PEG-lipid comprises about 2.0 mol % to about 2.5 mol % of the total lipid present in the particle. In some embodiments, the PEG-lipid comprises about 1 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of the total lipid present in the particle.

### Phospholipid

As used herein, a “phospholipid” refers to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

In some embodiments, the described LNP composition comprises a phospholipid. In some embodiments, the phospholipid comprises a lipid selected from the group consisting of: phosphatidylcholine (PC), phosphatidylethanolamine amine, glycerophospholipid, sphingophospholipids, Guriserohosuhono, sphingolipids phosphono lipids, natural lecithins, and hydrogenated phospholipid. In some embodiments, the phospholipid comprises a phosphatidylcholine. Exemplary phosphatidylcholines include, but are not limited to, soybean phosphatidylcholine, egg yolk phosphatidylcholine (EPC), distearoylphosphatidylcholine, 1,2-distearoyl-sn-glycero-3-phosphocholine (DSPC), dipalmitoyl phosphatidylcholine, dipalmitoyl-sn-glycero-3-phosphocholine (DPPC), 2-Oleoyl-1-palmitoyl-sn-glycero-3-phosphocholine (POPC), dimyristoyl phosphatidylcholine (DMPC), and dioleoyl phosphatidylcholine (DOPC). In certain specific embodiments, the phospholipid is DSPC.

In some embodiments, the phospholipid comprises a phosphatidylethanolamine amine. In some embodiments, the phosphatidylethanolamine amine is distearoyl phosphatidylethanolamine (DSPE), dipalmitoyl phosphatidyl ethanolamine (DPPE), 1,2-dioleoyl-sn-glycero-3-phosphoethanolamine (DOPE), dimyristoyl phosphoethanolamine (DMPE), 16-O-Monome Le PE, 16-O-dimethyl PE, 18-1-trans PE, palmitoyl oleoyl-phosphatidylethanolamine (POPE), or 1-stearoyl-2-oleoyl-phosphatidyl ethanolamine (SOPE). In some embodiments, the phospholipid comprises a glycerophospholipid. In some embodiments, the glycerophospholipid is plasmalogen, phosphatidate, or phosphatidylcholine. In some embodiments, the glycerophospholipid is phosphatidylserine, phosphatidic acid, phosphatidylglycerol, phosphatidylinositol, palmitoyl oleoyl phosphatidylglycerol (POPG), or lysophosphatidylcholine. In some embodiments, the phospholipid comprises a sphingophospholipid. In some embodiments, the sphingophospholipid is sphingomyelin, ceramide phosphoethanolamine, ceramide phosphoglycerol, or ceramide phosphoglycerophosphoric acid. In some embodiments, the phospholipid comprises a natural lecithin. In some embodiments, the natural lecithin is egg yolk lecithin or soybean lecithin. In some embodiments, the phospholipid comprises a hydrogenated phospholipid. In some embodiments, the hydrogenated phospholipid is hydrogenated soybean phosphatidylcholine. In some embodiments, the phospholipid is selected from the group consisting of: phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

In some embodiments, the phospholipid comprises a lipid selected from: 1,2-distearoyl-sn-glycero-3-phosphocholine (DSPC). 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 2-Oleoyl-1-pahnitoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-di arachidonoyl-sn-glycero-3-phosphocholine, 1,2-di docosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diaracliidonoyl-sn-gly cero-3-phosphoethanol amine, 1,2-di docosahexaenoyl-sn-glycero-3-phosphoethanolamine, 1,2-diol eoy I-sn-glycero-3-phospho-rac-(1-glycerol) sodium salt (DOPG), and sphingomyelin.

A phospholipid can comprise a phospholipid moiety and one or more fatty acid moieties. A phospholipid moiety can comprise phosphatidyl choline, phosphatidyl ethanolamine, phosphatidyl glycerol, phosphatidyl serine, phosphatidic acid, 2-lysophosphatidyl choline, or a sphingomyelin. A fatty acid moiety can comprise lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, or docosahexaenoic acid. In some specific embodiments, a phospholipid can be functionalized with or cross-linked to one or more alkynes, which may undergo a copper-catalyzed cycloaddition upon exposure to an azide.

In some embodiments, the LNP composition comprises a plurality of phospholipids, for example, at least 2, 3, 4, 5, or more distinct phospholipids. In some embodiments, the phospholipid comprises from 1 mol % to 20 mol % of the total lipid present in the particle. In some embodiments, the phospholipid comprises from about 5 mol % to about 15 mol % of the total lipid present in the particle. In some embodiments, the phospholipid comprises from about 8 mol % to about 12 mol % of the total lipid present in the particle. In some embodiments, the phospholipid comprises from about 9 mol %, 10 mol %, or 11 mol % of the total lipid present in the particle.

### Cholesterol

In some embodiments, the LNP composition comprises a cholesterol or a derivative thereof. In some embodiments, the LNP composition comprises a structural lipid. The structural lipid can be selected from steroid, sterol, alkyl resoreinol, cholesterol or derivative thereof, fecosterol, sitosterol, ergosterol, campesterol, stigmasterol, brassicasterol, tomatidine, tomatine, ursolic acid, alphatocopherol, and a combination thereof. In some embodiments, the structural lipid is a corticosteroid such as prednisolone, dexamethasone, prednisone, and hydrocortisone. In some embodiments, the cholesterol or derivative thereof is cholesterol, 5-heptadecylresorcinol, or cholesterol hemisuccinate. In some embodiments, the cholesterol or derivative thereof is cholesterol.

In some embodiments, the cholesterol or derivative thereof is a cholesterol derivative. In some embodiments, the cholesterol derivative is a polar cholesterol analogue. In some embodiments, the polar cholesterol analogue is 5a-cholestanol, 513-coprostanol, cholesteryl-(2′-hydroxy)-ethyl ether, cholestelyl-(4′-hydroxy)-butyl ether, or 6-ketocholestanol. In some embodiments, the polar cholesterol analogue is cholesteryl-(4′-hydroxy)-butyl ether. In some embodiments, the cholesterol derivative is a non-polar cholesterol analogue. In some embodiments, the non-polar cholesterol analogue is 5acholestane, cholestenone, 5α-cholestanone, 50-cholestanone, or cholestetyl decanoate.

In some embodiments, the cholesterol or the derivative thereof comprises from 20 mol % to 50 mol % of the total lipid present in the particle. In some embodiments, the cholesterol or the derivative thereof comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %. about 45 mol %. about 46 mol %, about 47 mol %, about 48 mol %, or about 50 mol % of the total lipid present in the particle.

### Phosphate Charge Neutralizer

In some embodiments, the LNP described herein comprises a phosphate charge neutralizer. In some embodiments, the phosphate charge neutralizer comprises arginine, asparagine, glutamine, lysine, histidine, cationic dendrimers, polyamines, or a combination thereof. In some embodiments, the phosphate charge neutralizer comprises one or more nitrogen atoms. In some embodiments, the phosphate charge neutralizer comprises a polyamine. In some embodiments, the polyamine is 1,3-propanediamine, spermine, spermidine, Norspermidine, Tris(2-aminoethy)amine, Cyclen, 1,4,7-Triazacyclononane, 1,1,1-Tris(aminomethyl)ethane, Diethylenetriamine, Triethylenetetramine, or a combination thereof. In some embodiments, the polyamine is 1,3-propanediamine, 1,4-butanediamine, spermine, spermidine, or a combination thereof. In some embodiments, the N/P ratio for the phosphate charge neutralizer is from 0.01 to 10. In some embodiments, the N/P ratio for the phosphate charge neutralizer is from about 0.05 to about 2. In some embodiments, the N/P ratio for the phosphate charge neutralizer is from about 0.1 to about 1. In some embodiments, the N/P ratio for the phosphate charge neutralizer is about 0.1, about 0.2, about 0.3, about 0.4, about 0.5, about 0.6, about 0.7, about 0.8, about 0.9, or about 1. In some embodiments, the NIP ratio for the phosphate charge neutralizer is about 0.25, 0.5, or 0.75.

### Antioxidants

In some embodiments, the LNP described herein comprises one or more antioxidants. In some embodiments, the one or more antioxidants function to reduce a degradation of the cationic lipids, the payload, or both. In some embodiments, the one or more antioxidants comprise a hydrophilic antioxidant. In some embodiments, the one or more antioxidants is a chelating agent such as ethylenediaminetetraacefic acid (EDTA) and citrate. In some embodiments, the one or more antioxidants is EDTA. In some embodiments, the one or more antioxidants comprise a lipophilic antioxidant. In some embodiments, the lipophilic antioxidant comprises a vitamin E isomer or a polyphenol. In some embodiments, the one or more antioxidants are present in the LNP composition at a concentration of at least 1 mM, at least 10 mM, at least 20 mM, at least 50 mM, or at least 100 mM. In some embodiments, the one or more antioxidants are present in the particle at a concentration of about 20 mM.

### Payload

The LNPs described herein can be designed to deliver a payload, such as a therapeutic agent, or a target of interest. In some embodiments, an LNP described herein encloses one or more components of a base editor system as described herein. For example, a LNP may enclose one or more of a guide RNA, a nucleic acid encoding the guide RNA, a vector encoding the guide RNA, a base editor fusion protein, a nucleic acid encoding the base editor fusion protein, a programmable DNA binding domain, a nucleic acid encoding the programmable DNA binding domain, a deaminase, a nucleic acid encoding the deaminase, or all or any combination thereof. In some embodiments, the nucleic acid is a DNA. In some embodiments, the nucleic acid is a RNA, for example, a mRNA.

Additional exemplary therapeutic agents include, but are not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, enzymatic nucleic acids, antisense nucleic acids, triplex forming oligonucleotides, antisense DNA or RNA compositions, chimeric DNA:RNA compositions, allozymes, aptamers, ribozyme, decoys and analogs thereof, plasmids and other types of expression vectors, and small nucleic acid molecules, RNAi agents, short interfering nucleic acid (siNA), messenger ribonucleic acid (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), aiRNA (asymetrical interfering RNA), and siRNA with 1, 2 or more mismatches between the sense and anti-sense strand to relevant cells and/or tissues, such as in a cell culture, subject or organism. Therapeutic agents can be purified or partially purified, and can be naturally occurring or synthetic, or chemically modified. In some embodiments, the therapeutic agent is an RNAi agent, short interfering nucleic acid (siNA), short interfering RNA (siRNA), double-stranded RNA (dsRNA), micro-RNA (miRNA), or a short hairpin RNA (shRNA) molecule. In some embodiments, the therapeutic agent is an mRNA.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), or a Dicer-Substrate dsRNA.

### Other Lipids

In some embodiments, the disclosed LNP compositions comprise a helper lipid. In some embodiments, the disclosed LNP compositions comprise a neutral lipid. In some embodiments, the disclosed LNP compositions comprise a stealth lipid. In some embodiments, the disclosed LNP compositions comprises additional lipids.

As used herein, neutral lipids suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), 1,2-dipalmitoyl-sn-glycero-3-phosphocholine (DPPC), 1,2-distearoyl-sn-glycero-3-phosphocholine (DSPC), phosphocholine (DOPC), 1,2-dimyristoyl-sn-glycero-phosphocholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoylsn-glycero-3-phosphocholine (DBPC), 1-stearoyl-2-palmitoyl phosphatidylcholine (SPPC), 1,2-dieicosenoyl-sn-glycero-3-phosphocholine (DEPC), 2-Oleoyl-1-palmitoyl-sn-glycero-3-phosphocholine (POPC), lysophosphatidyl choline, 1,2-dioleoyl-sn-glycero-3-phosphoethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In some embodiments, the neutral phospholipid is selected from the group consisting of DSPC and dimyristoyl phosphatidyl ethanolamine (DMPE). In some embodiments, the neutral phospholipid is DSPC. Neutral lipids can function to stabilize and improve processing of the LNPs.

Helper lipids can refer to lipids that enhance transfection (e.g. transfection of the nanoparticle (LNP) comprising the composition as provided herein, including the biologically active agent). The mechanism by which the helper lipid enhances transfection includes enhancing particle stability. In some embodiments, the helper lipid enhances membrane fusogenicity. Helper lipids can include steroids, sterols, and alkyl resorcinols. Helper lipids suitable for use in the present disclosure can include, but are not limited to, cholesterol, 5-heptadecylresorcinol, and cholesterol hemisuccinate. In some embodiments, the helper lipid is cholesterol. In some embodiments, the helper lipid is be cholesterol hemisuccinate.

Stealth lipids can refer to lipids that alter the length of time the nanoparticles can exist in vivo (e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and I-Toekstra et al, Biochimica et Biophysica Acta 1660 (2004) 41-52. Additional suitable PEG lipids are disclosed, e.g., in WO 2006/007712.

In some embodiments, the stealth lipid is a PEG-lipid. In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly N-(2-hydroxypropyl)methacrylamide]. Stealth lipids can comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### LNP Formulations

The LNPs described herein can be designed for one or more specific applications or targets. The elements of a nanoparticle (LNP) composition or a composition can be selected based on a particular application or target, and/or based on the efficacy, toxicity, expense, ease of use, and availability. Similarly, the particular formulation of a nanoparticle composition is a composition comprising one or more described lipids. may be selected for the particular application or target. Suitable phosphate charge neutralizers to be used in formulations include, but are not limited to, Spermidine and 1,3-propanediamine.

The described LNP formulations can be designed for one or more specific applications or targets. For example, a nanoparticle composition may be designed to deliver a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ.

The amount of a therapeutic agent in an LNP composition may depend on the size, composition, desired target and/or application, or other properties of the nanoparticle composition. For example, the amount of an RNA comprised in a nanoparticle composition may depend on the size, sequence, and other characteristics of the RNA. The relative amounts of a therapeutic agent and other elements (e.g., lipids) in a nanoparticle composition may also vary. In some embodiments, the wt/wt ratio of the lipid component to a therapeutic agent in a nanoparticle composition may be from about 5:1 to about 60:1, such as about 5:1. 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic agent may be from about 10:1 to about 40:1. In certain embodiments, the wt/wt ratio is about 20:1. The amount of a therapeutic agent in a nanoparticle composition can be measured using absorption spectroscopy (e.g., ultraviolet-visible spectroscopy).

In some embodiments, an LNP composition comprises one or more nucleic acids such as RNAs. In some embodiments, the one or more RNAs, lipids, and amounts thereof may be selected to provide a specific NIP ratio. The NIP ratio can be selected from about 1 to about 30. The N/P ratio can be selected from about 2 to about 10. In some embodiments, the NIP ratio is from about 0.1 to about 50. In some embodiments, the N/P ratio is from about 2 to about 8. hi some embodiments, the NIP ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. hi some embodiments, the N/P ratio is about 2, about 2.5, about 3, about 3.5, about 4, about 4.5, about 5, about 5.5, about 6, or about 6.5. In some embodiments, the NIP ratio is from about 4 to about 6. In some embodiments, the NIP ratio is about 4, about 4.5, about 5, about 5.5, or about 6.

In some embodiments, the LNPs are formed with an average encapsulation efficiency ranging from about 50% to about 70%, from about 70% to about 90%, or from about 90% to about 100%. In some embodiments, the LNPs are formed with an average encapsulation efficiency ranging from about 75% to about 95%.

In another aspect, provided herein is a lipid nanoparticle (LNP) comprising the composition as provided herein. As used herein, a lipid nanoparticle (LNP) composition or a nanoparticle composition is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm. In some embodiments, a liposome having a lipid bilayer with a diameter of 500 rim or less. In some embodiments, the LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 rim to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm- or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

In some embodiments, an LNP may be made from cationic, anionic, or neutral lipids. In some embodiments, an LNP may comprise neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N[1-(2,3-dioleyloxy)propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N—(N′,N′-dimethylaminoethane)-carbamoyl]cholesterol), DOTAP-cholesterol, GAP-DMORIE-DPyPE, and GL67A-DOPE-DMPE (2-Bis(dimethylphosphino)ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce a LNP. In some embodiments, the polynucleotide may be combined with lipid(s) in a wide range of molar ratios to produce an LNP.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioallcyl, alkylsulfonyl, alkylsulfonylalkyl, arylsulfonylalkyl, alkoxy, aryloxy, aralkoxy, aminocarbonyl, alkylaminocarbonyl, aiylaminocarbonyl, alkoxycarbonyl, aryloxycarbonyl, haloalkyl, amino, trifluoromethyl, cyano, nitro, alkylamino, arylamino, alkylaminoalkyl, aiylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary●substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinal), skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl. C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —C₁₋₁₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkenyl” refers to a type of alkyl group in which at least one carbon-carbon double bond is present. In one embodiment, an alkenyl group has the formula —C(R)═CR², wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopenteny 1. Polycyclic radicals include, for example, adamantyl, 1,2-dihydronaphthalenyl, 1,4-dihydronaphthalenyl, tetrainyl, decalinyl, 3,4-dihydronaphthalenyl-.1 (2H)— one. spiro[2.2]pentyl, norbomyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloallcyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzofused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl. thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4Hpyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazoli diny I, 3-az.abicy cl o[3. 1.0]hexany 1,3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, futyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furaz.anyl, benzofuraz.anyl, benzothiophenyl, benzothiazolyl, benzoxazolyl, quinazolinyl, quinoxalinyl, naphthyridinyl, and furopyridinyl. The foregoing groups are either C-attached (or Clinked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heterowyl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “teterocycloalkylene” can refer to a divalent heterocycloalkyl group.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

As used herein, the “N/P ratio” is the molar ratio of ionizable (e.g., in the physiological pH range) nitrogen atoms in a lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH5, about pH 6, about pH 7, about pH 7.5, or about pH 8 or higher. The physiological pH range can include, for example, the pH range of different cellular compartments (such as organs, tissues, and cells) and bodily fluids (such as blood, CSF, gastric juice, milk, bile, saliva, tears, and urine). In certain specific embodiments, the physiological pH range refers to the pH range of blood in a mammal, for example, from about 7.35 to about 7.45. Similarly, for phosphate charge neutralizers that have one or more ionizable nitrogen atoms, the N/P ratio can refer to a molar ratio of ionizable nitrogen atoms in the phosphate charge neutralizer to the phosphate groups in a nucleic acid. In some embodiments, ionizable nitrogen atoms refer to those nitrogen atoms that are ionizable within a pH range between 5 and 14.

It is further contemplated that the LNP formulations that encapsulate the gRNAs and mRNAs drug substances described herein may be modified to include GalNAc lipid formulations such as those disclosed in co-owned U.S. patent application Ser. No. 17/192,709 filed on Mar. 4, 2021 with a parallel PCT application being filed on same date having International Application Number PCT/US21/20955. Employment of such GalNAc lipid formulations are understood to be capable of enhancing LNP uptake in LDL-R deficient cells, such as those associated with heterozygous and homozygous familial hypercholesterolemia patient populations.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

As used herein, amino lipids can contain at least one primary, secondary or tertiary amine moiety that is protonatable (or ionizable) between pH range 4 and 14. In some embodiments, the amine moiety/moieties function as the hydrophilic headgroup of the amino lipids. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLipids). The amino lipids that constitute iLNPs can be called ionizable amino lipids (iLipids). The amino lipid can be an iLipid or a cLipid at physiological pH.

Additional LNPs can be found in PCT Application Publication NO. WO2021/207712, which is hereby incorporated by reference in its entirety.

### GalNAc-LNPs

In some aspects, described herein are methods of preparing a formulation comprising GalNAc-lipid nanoparticles (GalNAc-LNPs). In some embodiments, the nanoparticles comprise (i) one or more nucleic acid active agents, (ii) one or more lipid excipients selected from sterol or a derivative thereof, a phospholipid, a stealth lipid, and an amino lipid, and/or (iii) a GalNAc-lipid receptor targeting conjugate. In some embodiments, the methods can comprise providing a first solution comprising the one or more nucleic acid active agents in aqueous buffer. In some embodiments, the methods can comprise providing a second solution comprising (i) at least one of the one or more lipid excipients and (ii) at least a portion of the receptor targeting conjugate in a water-miscible organic solvent. In some embodiments, the methods can comprise combining an antioxidant with said first solution; In some embodiments, the methods can comprise mixing said first solution and said second solution. In some embodiments, the methods can comprise incubating a mixture of said first and second solutions to form GalNAc-LNP. In some embodiments, the methods can comprise carrying out one or more processes selected from dilution, buffer exchange, concentration, filtration, freezing, thawing, incubation and GalNAc-LNP evaluation.

In some embodiments steps of the methods are performed simultaneously. In some embodiments, steps of the methods are performed sequentially.

In some embodiments, the aqueous buffer comprises polyethylene glycol. In some embodiments, the polyethylene glycol has a number average molecular weight ranging from about 200 to about 1000 (for example, about 200, about 400, about 500, about 600, or about 1000). In some embodiments, the methods further comprise diluting GalNAc-Lipid in an aqueous solution to produce a diluted GalNAc-LNP solution. In some embodiments, the GalNAc-LNP is configured for direct administration to a subject. In some embodiments, the methods further comprise diluting said GalNAc-LNPs in a solution one or more times. In some embodiments, the methods further comprise exchanging said water-miscible organic solvent with a buffer solution one or more times. In some embodiments, the methods further comprise concentrating said GalNAc-LNPs. In some embodiments, the concentrating comprises passing said GalNAc-LNPs through a membrane. In some embodiments, the methods further comprise a second concentrating process, wherein the second concentrating comprises concentrating said GalNAc-LNP by passing the exchanging buffer through a membrane.

In some embodiments, the methods further comprise filtering said GalNAc-LNPs through a membrane. In some embodiments, the methods further comprise a second incubation after step e, wherein incubation occurs from about 1 minute to about 120 minutes. In some embodiments, the methods further comprise storing said GalNAc-LNPs at a temperature of about −80 degrees Celsius (° C.) to about 25° C. In some embodiments, the methods further comprise storing said GalNAc-LNPs at a temperature of about −80 degrees Celsius (° C.) or from about 2° C. to about 8° C.

In some embodiments, the methods further comprise comprising (i) thawing stored GalNAc-LNPs (ii) pooling GalNAc-LNPs (iii) diluting GalNAc-LNPs in a solution and (iv) filtering said GalNAc-LNPs through a membrane prior to administering a dose of said GalNAc-LNPs to a subject. In some embodiments, the order of performing step (iii) and (iv) are reversed. In some embodiments, said miscible organic solvent is ethanol. In some embodiments, said antioxidant is ethylenediaminetetraacetic acid (EDTA). In some embodiments, said second solution comprises all the receptor targeting conjugate. In some embodiments, at least a portion of said receptor targeting conjugate is combined with one or more lipids prior to the mixing step.

In some embodiments, the mixing occurs in an inline mixer, cross mixer, or T mixer apparatus. In some embodiments, the mixing comprises laminar mixing, vortex mixing, turbulent mixing, or a combination thereof. In some embodiments, the methods further comprise using a tangential flow filtration (TFF) process to concentrate said GalNAc-LNPs. In some embodiments, the methods further comprise using a chromatography, dialysis, or a TFF process to perform buffer exchange.

In some embodiments, the receptor targeting conjugate comprises one or more N-acetylgalactosamine (GalNAc) or GalNAc derivatives. In some embodiments, the mixing is performed by an inline mixing apparatus having a first mixing chamber that includes a first port that separately introduces said first solution to said first mixing chamber and a second port that separately and simultaneously introduces said second solution into said first mixing chamber. In some embodiments, said first solution comprises RNA. In some embodiments, a concentration (mol %) of said GalNAc-lipid receptor targeting conjugate is about 0.01 mol % to about 10 mol %. In some embodiments, said neutral lipid is distearoylphosphatidylcholine (DSPC). In some embodiments, said stealth lipid is polyethylene glycol-dimyristoyl glycerol (PEG-DMG). In some embodiments, said stealth lipid concentration in said second solution is 0 mol % to about 5 mol %. In some embodiments, said nucleic acid agent concentration is about 0.1 to about 5 mg/mL (e.g. about 0.1, 0.25, 0.5, 0.75, 1, 1.5, 2, 2.5, 3, 4, or 5 mg/mL). In some embodiments, said mixture is incubated for about 1 minute to about 24 hours. In some embodiments, said mixture is incubated for about 1 minute to about 120 minutes. In some embodiments, said mixture is incubated for about 1 hour. In some embodiments, a final GalNAc-LNP solution comprises Tris buffer.

In some embodiments, a final GalNAc-LNP solution further comprises a cryoprotectant. In some embodiments, said cryoprotectant is sucrose. In some embodiments, a concentration of said cryoprotectant in said final solution is about 0.1 mM to about 500 mM. In some embodiments, a concentration of said cryoprotectant in said final solution is about 150 mM to about 500 mM. In some embodiments, a concentration of said cryoprotectant is in said final solution is about 300 mM.

In some embodiments, GalNAc-LNPs are stored at a temperature of about −80 degrees Celsius (° C.). In some embodiments, a final GalNAc-LNP solution does not further comprise a cryoprotectant. In some embodiments, said GalNAc-LNPs are stored from about 2° C. to about 8° C. In some embodiments, said GalNAc-LNP are in a solution with a pH from about 6 to about 9. In some embodiments, said GalNAc-LNP are in solution with a pH of about 7-8 (e.g. 7-8, 7.2-7.8, 7.3-7.7, or 7.4-7.6).

In some embodiments, the methods further comprise introducing said receptor targeting conjugate in said second solution at a concentration of at least 0.01 (e.g. at least 0.01, 0.05, 0.1, or 0.5) mol % of total volume. In some embodiments, the methods further comprise introducing said receptor targeting conjugate in said second solution at a concentration of at least 1 mol % of total volume. In some embodiments, the methods further comprise introducing said receptor targeting conjugate in said second solution at a concentration of at least 3 mol % of total volume. In some embodiments, the methods further comprise introducing said receptor targeting conjugate in said second solution at a concentration of at least 5 mol % of total volume.

In some embodiments, the methods further comprise introducing said receptor targeting conjugate in said second solution at a concentration of at least 7 mol % of total volume. In some embodiments, the methods further comprise introducing said receptor targeting conjugate in said second solution at a concentration of at least 9 mol % of total volume. In some embodiments, the methods further comprise introducing said receptor targeting conjugate in said second solution at a concentration of at least 10 mol % of total volume.

In another aspect, described herein are GalNAc-LNPs which are capable of being prepared by methods described herein. In some embodiments, a distribution of GalNAc-lipid across said LNP is substantially uniform. In some embodiments, wherein a GalNAc-lipid is present in the GalNAc-LNP at a concentration of about 0.01-0.5 mol %.

In another aspect, described herein are GalNAc-LNPs comprising a receptor targeting conjugate which comprises a compound of Formula (II):

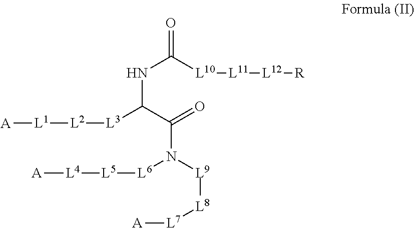

wherein, A is a receptor targeting moiety;
each L¹, L², L³, L⁴, L⁵, L⁶, L⁷, L⁸, L⁹, L¹⁰, and L¹², is independently substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, —(CH₂CH₂O)ₘ—, —(OCH₂CH₂)ₘ—, —O—, —S—, —S(═O)—, —S(═O)₂—, —S(═O)(═NR¹)—, —C(═O)—, —C(═N—OR¹)—, —C(═O)O—, —OC(═O)—, —C(═O)C(═O)—, —C(═O)N(R¹)—, —N(R¹)C(═O)—, —OC(═O)N(R¹)—, —N(R¹)C(═O)O—, —N(R¹)C(═O)N(R¹)—, —C(═O)N(R¹)C(═O)—, —S(═O)₂N(R¹)—, —N(R¹)S(═O)₂—, —N(R¹)—, —N(OR¹)—, —O[(P═O)O—]O—, —O[(P═O)S—]O—, —(CH₂)ₚ—O—, —O—(CH₂)ₚ—O—, —O—(CH₂)ₚ—, —S—S—, or a bond;
   L¹¹ is —(CH₂CH₂O)ₙ—, —(OCH₂CH₂)ₙ—, or a bond;
   each R¹ is independently H or substituted or unsubstituted C₁-C₆ alkyl;
   R is a lipophilic organic residue;
   m is an integer selected from 1 to 10;
   n is an integer selected from 1 to 200; and
p is an integer selected from 1 to 200.

In some embodiments, the receptor targeting conjugate is selected from the following:

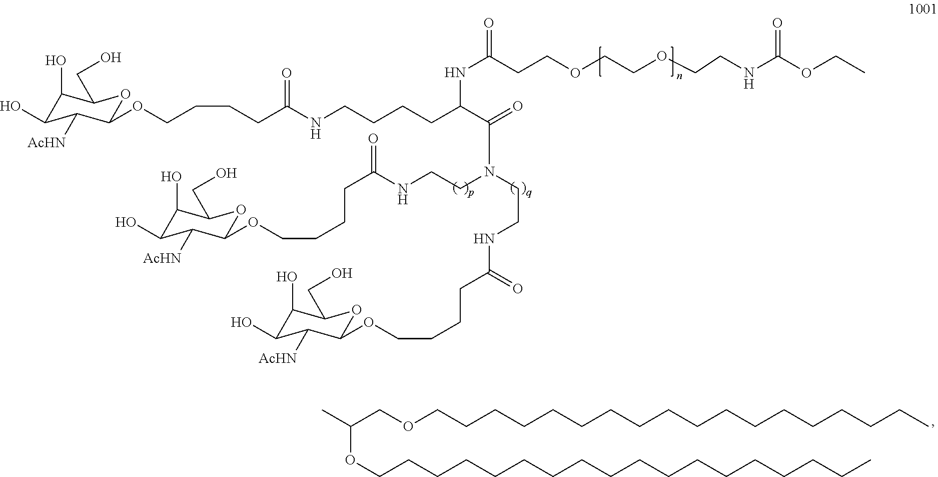

wherein each of the p and q is independently an integer from 1 to 5, and n is an integer from 1 to 50;

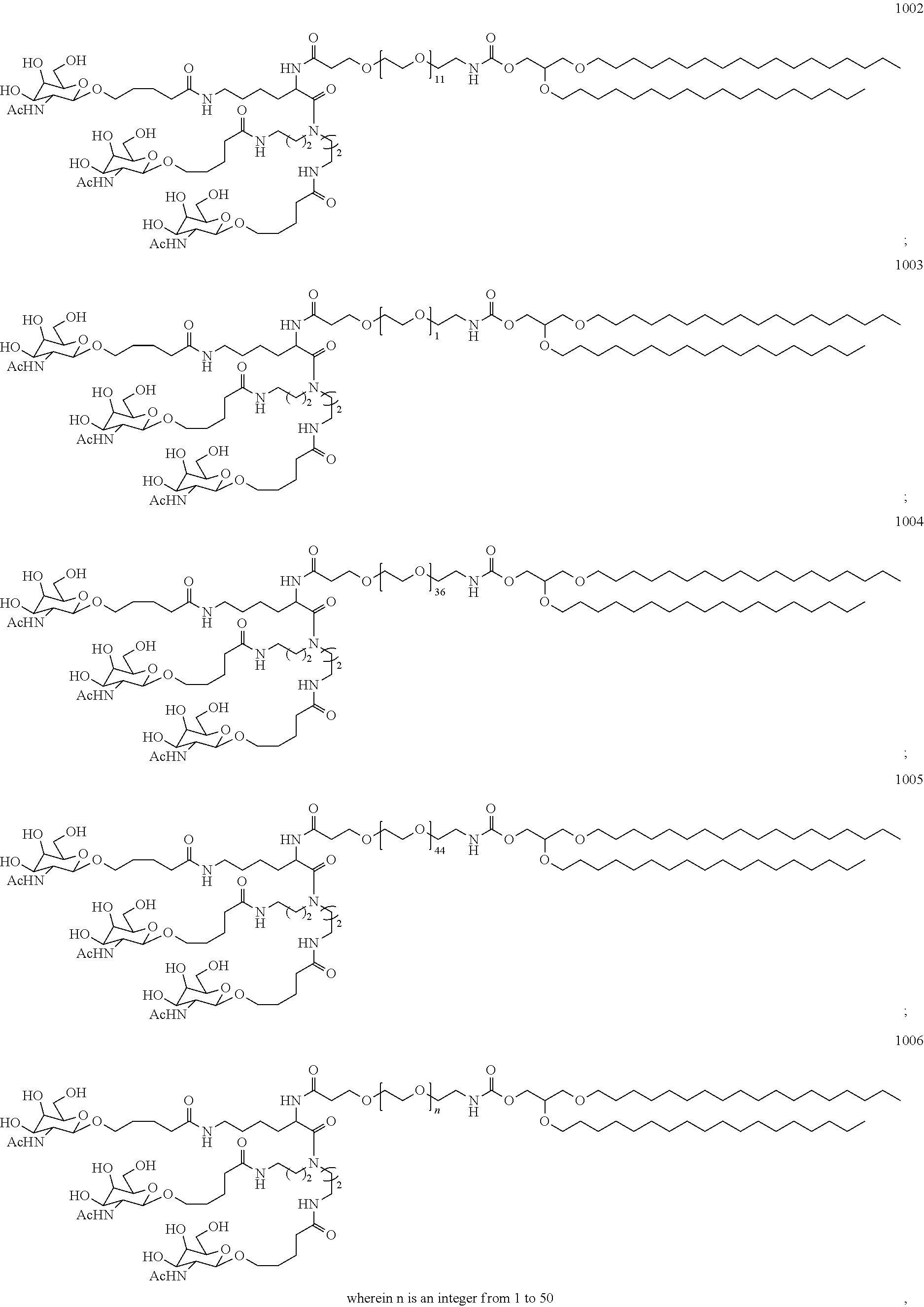

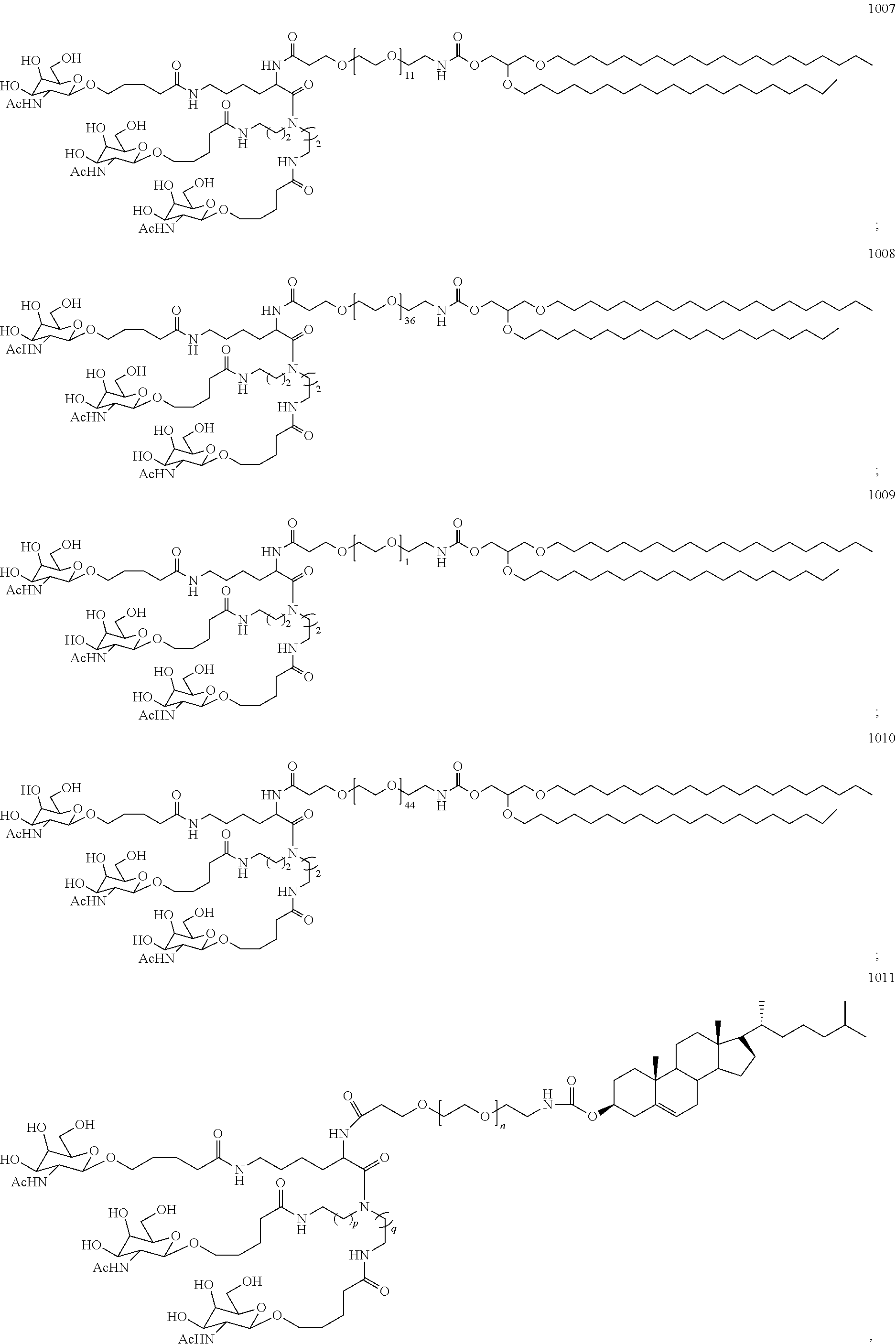

wherein each of the p and q is independently an integer from 1 to 5, and n is an integer from 1 to 50;

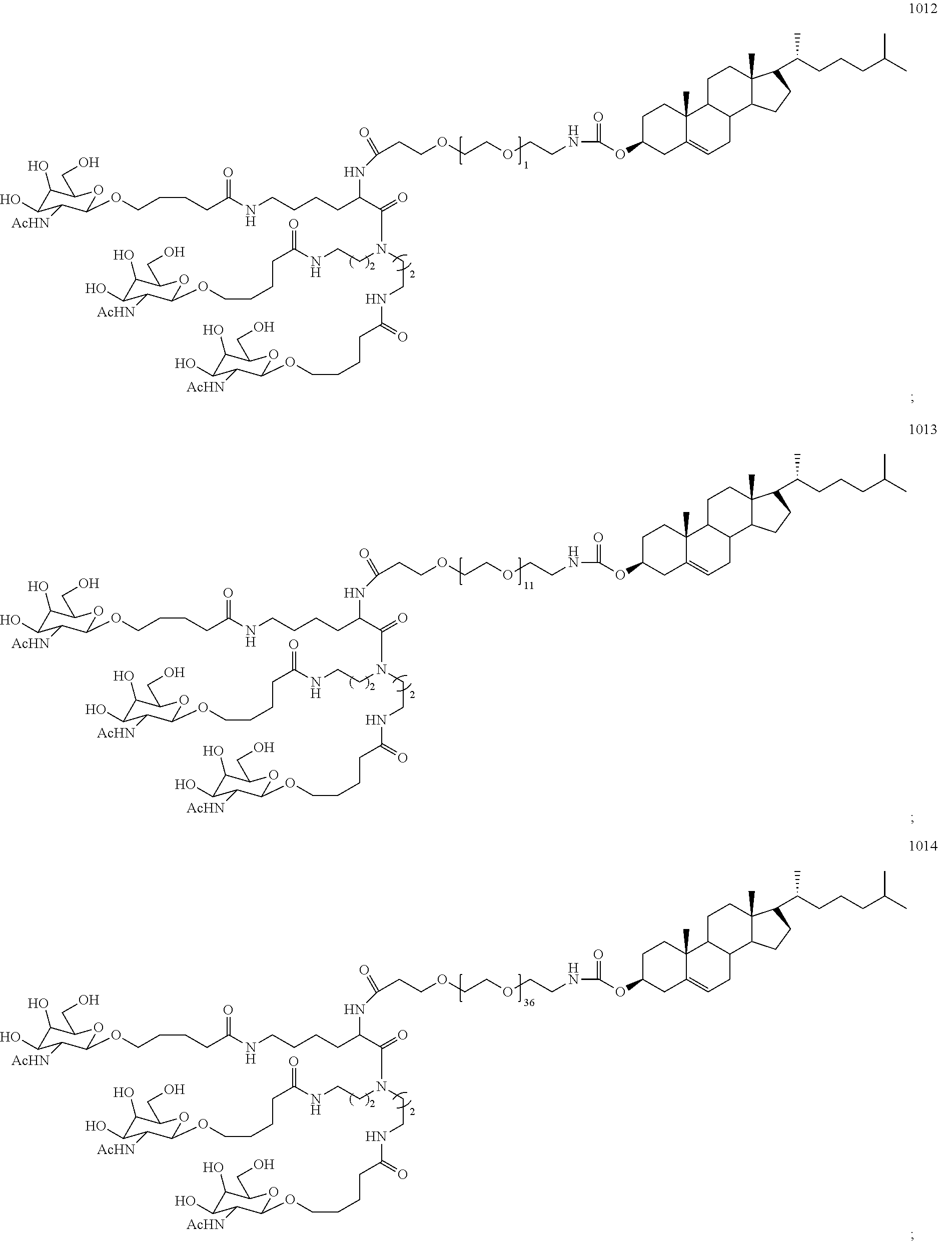

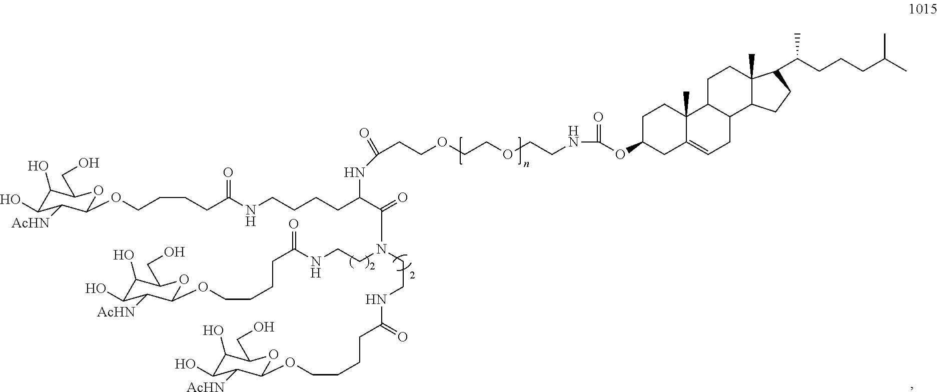

wherein n is an integer from 1 to 50;

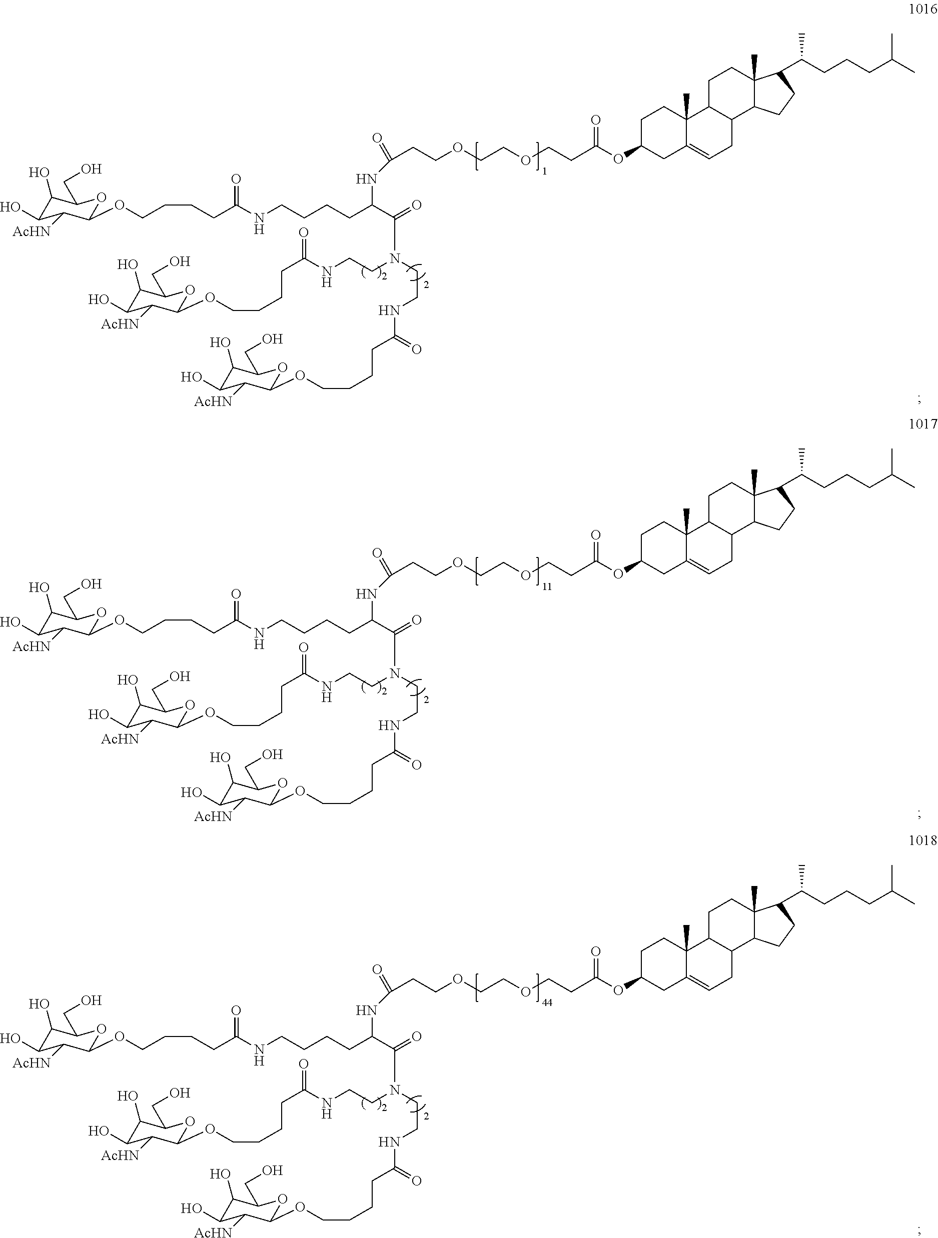

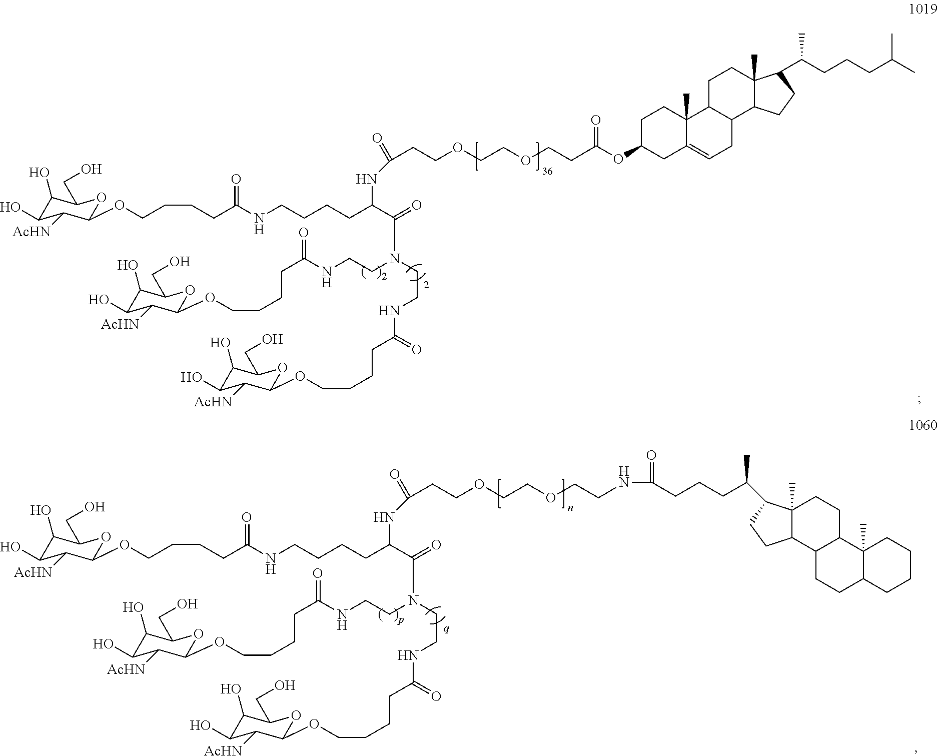

wherein each of the p and q is independently an integer from 1 to 5, and n is an integer from 1 to 50;

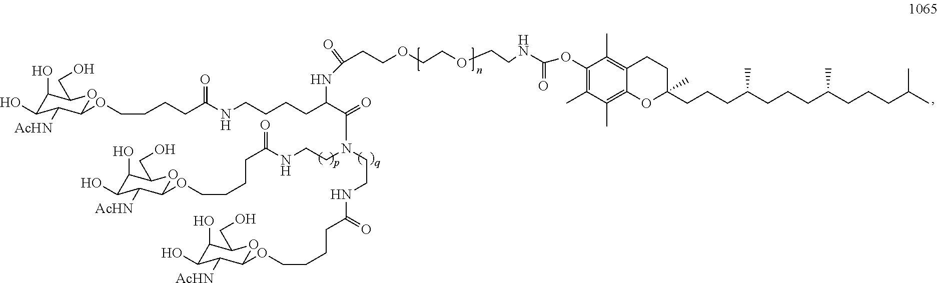

wherein each of the p and q is independently an integer from 1 to 5, and n is an integer from 1 to 50;

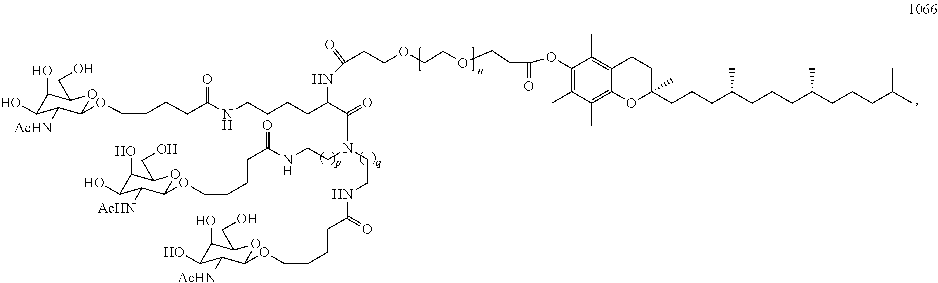

wherein each of the p and q is independently an integer from 1 to 5, and n is an integer from 1 to 50;

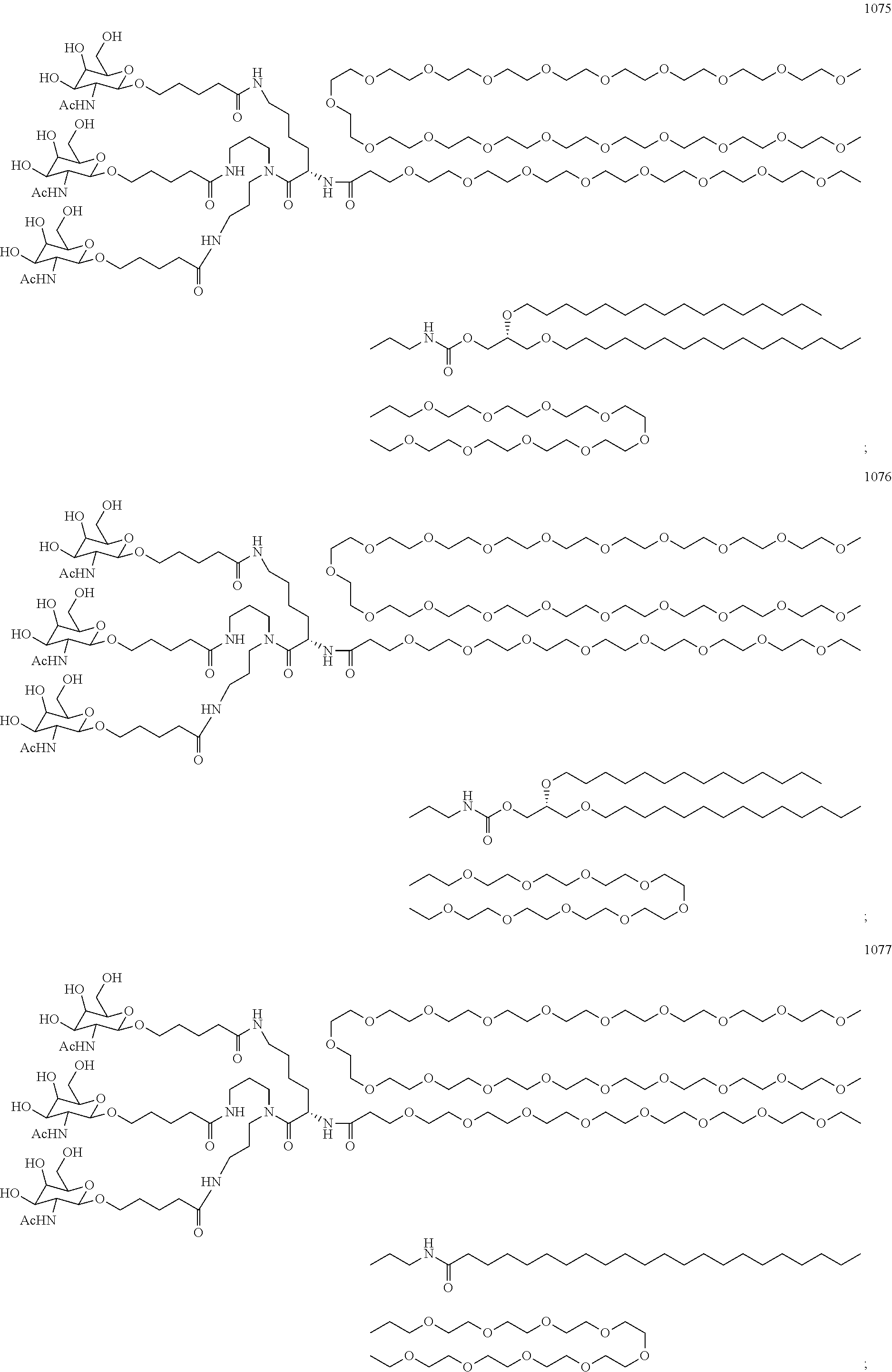

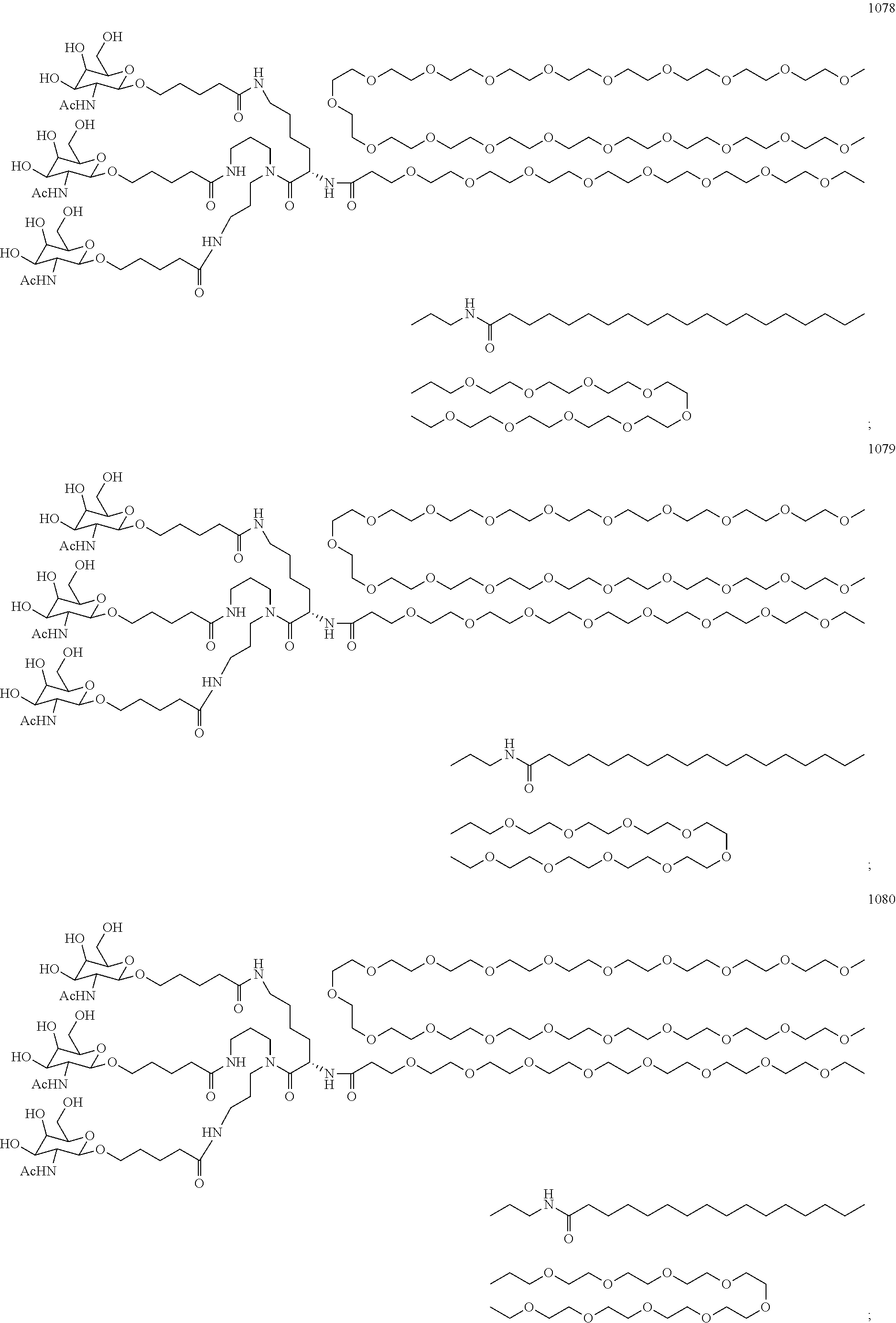

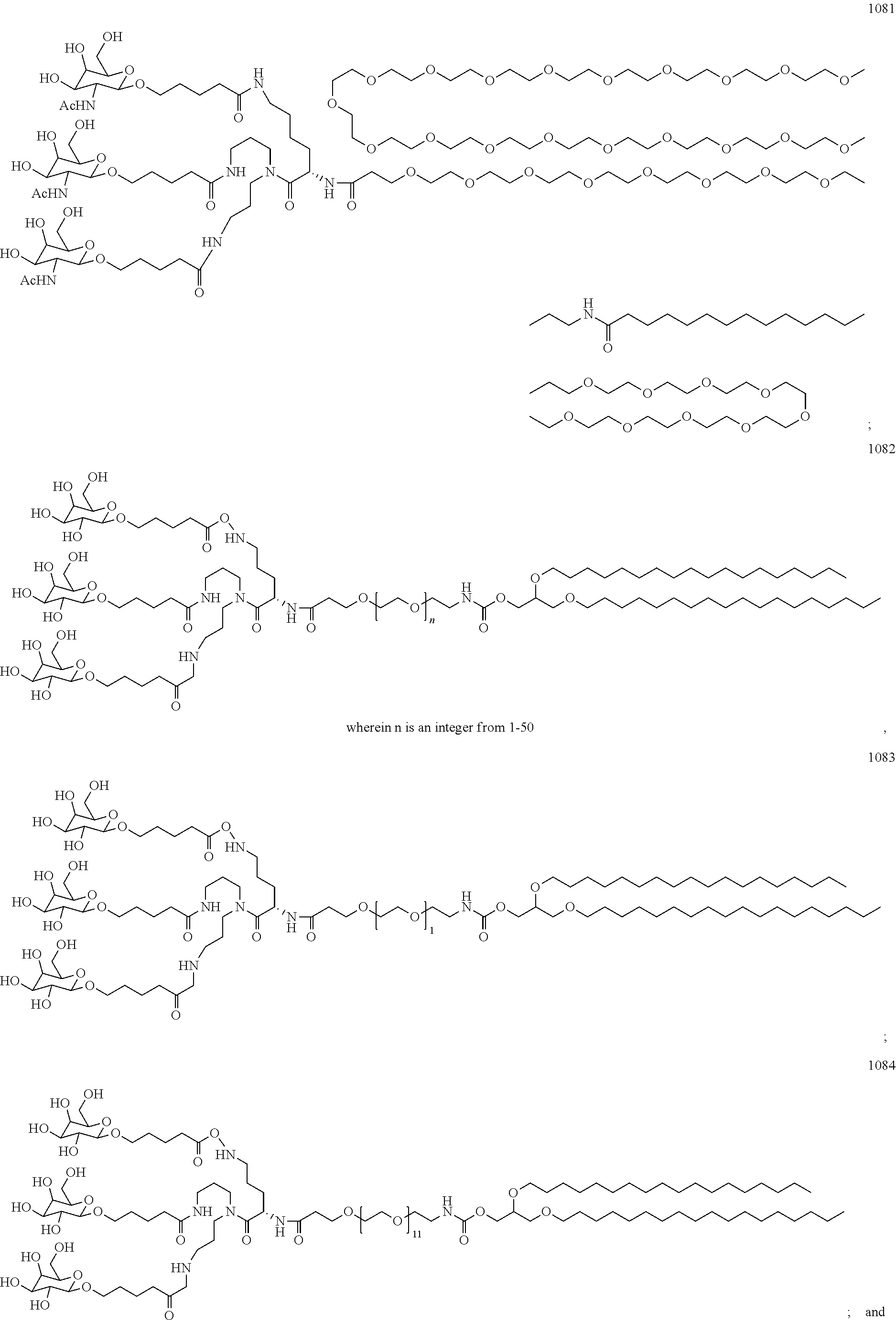

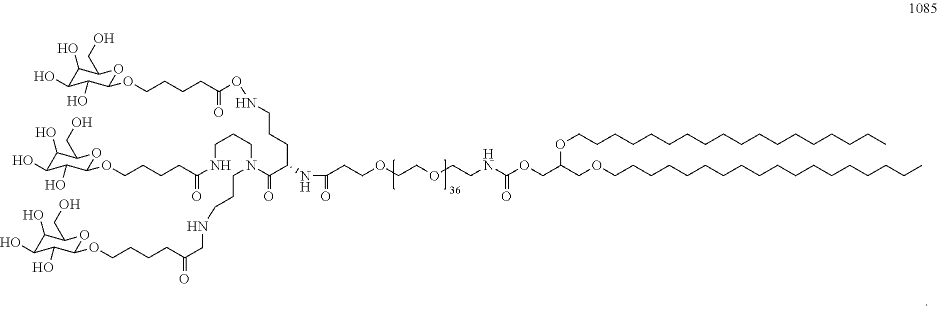

In some embodiments, the receptor targeting conjugate is:

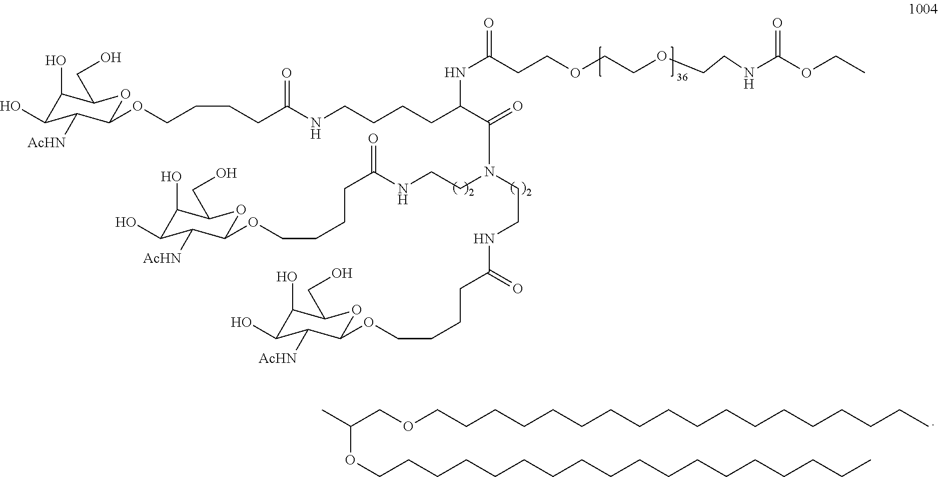

In another aspect described herein are pharmaceutical formulations comprising GalNAc-LNPs, the GalNAc-LNPs comprising:
(i) one or more nucleic acid active agents;
(ii) one or more lipid excipients selected from sterol or a derivative thereof, a phospholipid, a stealth lipid, and an amino lipid; and
(iii) a GalNAc-lipid receptor targeting conjugate, wherein the GalNAc-LNP comprises an excipient mol % ratio selected from Table 11.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

As used herein, amino lipids can contain at least one primary, secondary or tertiary amine moiety that is protonatable (or ionizable) between pH range 4 and 14. In some embodiments, the amine moiety/moieties function as the hydrophilic headgroup of the amino lipids. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLipids). The amino lipids that constitute iLNPs can be called ionizable amino lipids (iLipids). The amino lipid can be an iLipid or a cLipid at physiological pH.

### Method of Treatment

The present disclosure further provides a method for treating or preventing an atherosclerotic cardiovascular disease in a subject in need thereof, the method comprising administering to the subject a therapeutically effective amount of the guide nucleic acid, gene editing system, or the pharmaceutical composition disclosed herein. The subject in need thereof can be a subject that needs gene editing in ANGPTL3 gene. The gene editing system disclosed herein can achieve the editing in ANGPTL3 gene in a subject with high on-target efficiency but low off-target effect. The guide nucleic acid can direct the gene editor protein to affect a nucleobase alternation in an ANGPTL3 gene in the subject. The gene editing or base alteration achieved by the disclosed gene editing system can occur in at least 35% of whole liver cells in the subject as measured by next generation sequencing or Sanger sequencing, thereby treating or preventing the condition in the subject.

The methods disclosed herein can achieve at least about 20%, at least about 30%, at least about 40%, at least about 50%, at least about 60%, at least about 70%, at least about 80%, or at least about 90% of on-target editing in an ANGPTL3 gene in the subject. The methods disclosed herein can achieve at least 40% of on-target editing in an ANGPTL3 gene in the subject. The methods disclosed herein can achieve at least about 45% of on-target editing in an ANGPTL3 gene in the subject. The methods disclosed herein can achieve at least about 50% of on-target editing in an ANGPTL3 gene in the subject. The methods disclosed herein can achieve at least about 55% of on-target editing in an ANGPTL3 gene in the subject. The methods disclosed herein can achieve at least about 60% of on-target editing in an ANGPTL3 gene in the subject. The methods disclosed herein can achieve at least about 65% of on-target editing in an ANGPTL3 gene in the subject. The methods disclosed herein can achieve about 50% of on-target editing in an ANGPTL3 gene in the subject. The methods disclosed herein can achieve about 55% of on-target editing in an ANGPTL3 gene in the subject. The methods disclosed herein can achieve about 60% of on-target editing in an ANGPTL3 gene in the subject.

The methods disclosed herein can achieve at least about 40%, at least about 50%, at least about 60%, at least about 70%, at least about 80%, at least about 90%, or at least about 95% of reductions in plasma ANGPTL3 protein in a subject. The methods disclosed herein can achieve at least about 70% of reductions in plasma ANGPTL3 protein in a subject. The methods disclosed herein can achieve at least about 80% of reductions in plasma ANGPTL3 protein in a subject. The methods disclosed herein can achieve at least about 90% of reductions in plasma ANGPTL3 protein in a subject. The methods disclosed herein can achieve at least about 95% of reductions in plasma ANGPTL3 protein in a subject. The methods disclosed herein can achieve at least about 96% of reductions in plasma ANGPTL3 protein in a subject. The methods disclosed herein can achieve at least about 97% of reductions in plasma ANGPTL3 protein in a subject. The methods disclosed herein can achieve at least about 98% of reductions in plasma ANGPTL3 protein in a subject. The methods disclosed herein can achieve at least about 99% of reductions in plasma ANGPTL3 protein in a subject.

The methods disclosed herein can achieve at least about 20%, at least about 30%, at least about 40%, at least about 50%, at least about 60%, at least about 70%, or at least about 80% of reductions in HDL-C in a subject. The methods disclosed herein can achieve at least about 20% of reductions in HDL-C in a subject. The methods disclosed herein can achieve at least about 30% of reductions in HDL-C in a subject. The methods disclosed herein can achieve at least about 40% of reductions in HDL-C in a subject. The methods disclosed herein can achieve at least about 50% of reductions in HDL-C in a subject. The methods disclosed herein can achieve at least about 60% of reductions in HDL-C in a subject.

The methods disclosed herein can achieve at least about 10%, at least about 20%, at least about 30%, at least about 40%, at least about 50%, at least about 60%, or at least about 70% of reductions in plasma triglycerides in a subject. The methods disclosed herein can achieve at least about 10% of reductions in plasma triglycerides in a subject. The methods disclosed herein can achieve at least about 20% of reductions in plasma triglycerides in a subject. The methods disclosed herein can achieve at least about 30% of reductions in plasma triglycerides in a subject. The methods disclosed herein can achieve at least about 40% of reductions in plasma triglycerides in a subject. The methods disclosed herein can achieve at least about 50% of reductions in plasma triglycerides in a subject. The methods disclosed herein can achieve at least about 60% of reductions in plasma triglycerides in a subject.

The methods disclosed herein can effectively achieve on-target editing in an ANGPTL3 gene, reducing plasma ANGPTL3 protein, reducing HDL-C levels, and/or reducing plasma triglycerides in a subject without serious safety and/or toxicity concerns.

### EXAMPLES

The following examples are not intended to limit the scope of what the inventors regard as various aspects of the present invention.

Generally, as to the data presented herein, it should be understood that while various gRNA:mRNA ratios may be employed, unless otherwise expressly specified to the contrary, the gene editing systems set forth in the studies and data described and presented herein, employ 1:1 gRNA to mRNA ratio by weight. Similarly, while various dosing methods may be employed, unless otherwise expressly specified to the contrary, dosing specified by mg/kg in the studies and data described and presented herein refers to mg of the combined total gRNA and mRNA per kg weight of the dosed subject that is contained in the LNPs and intravenously dosed to the subject.

### Example 1. In-Vitro Potency Study Comparing PCSK9 Editing Percentage of Human Primary Liver Cells Vs. Non-Human Primary Liver Cells at Varied Doses of PCSK9 Base Editor

FIG. 1 presents data illustrating the percent editing of PCSK9 gene in primary human hepatocytes (PHH) as comparted to primary non-human primate hepatocytes (e.g., primary cyno hepatocytes (PCH)) using the LNP base editor formulation embodiments described herein. The vertical axis represents the percent editing of PCSK9 gene measured using next generation sequencing (NGS) to quantitate the A to G edits at the PCSK9 gene editing target site. The horizontal axis plots the log of the concentration of gRNA GA346 that the cells were exposed to. The LNP administered to the PHH and PCH cells comprised a payload of mRNA MA004 and guide RNA GA346 at 1:1 mRNA:gRNA weight ratio and titrated at different concentrations against multiple lots of primary human hepatocytes and primary non-human primate hepatocytes. The concentrations titrated against primary human hepatocytes extended from 3.025 to 3.469 nM of gRNA. The concentrations titrated against primary non-human hepatocytes extended from 9.035 to 11.68 nM of gRNA. For each group of cells, the percent editing of PCSK9 gene was analyzed at each gRNA concentration and plotted on the graph set forth in FIG. 1. The relative potency, as illustrated by FIG. 1, indicates that the formulation has notably greater potency in human primary liver cells than in non-human primate liver cells, which supports the likelihood that the formulation, when administered to humans, is capable of producing PCSK9 editing in human liver cells comparable or superior to those seen in the NHP studies described herein while using clinical doses meaningfully below those used in the NHP studies described herein.

### Example 2. In-Vivo Long-Term Non-Human Primate Study Dosed Intravenously with PCSK9 LNP Formulated with a Payload of ABE mRNA MA004 and Guide RNA GA346

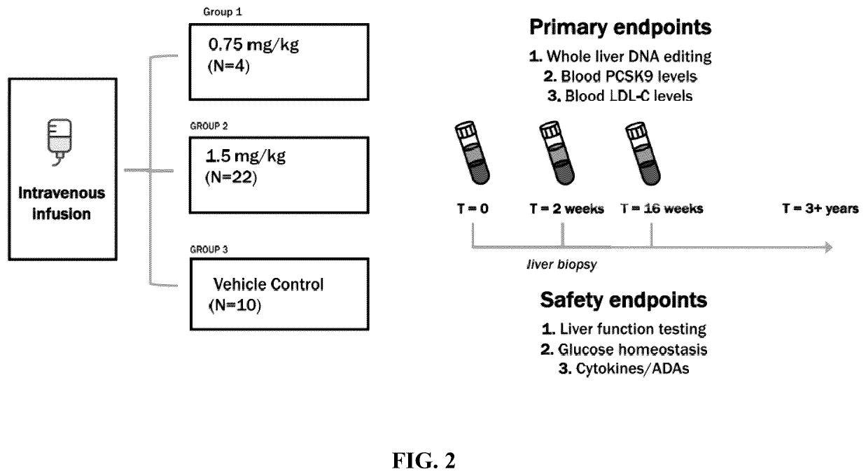

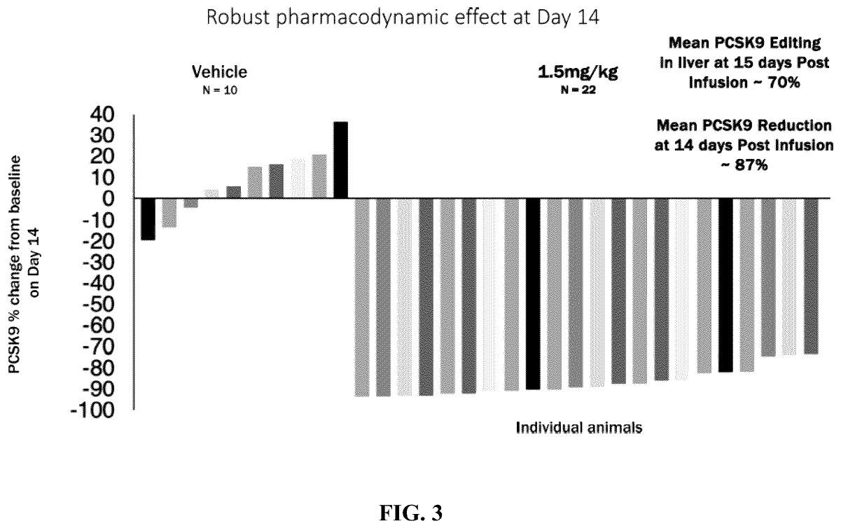

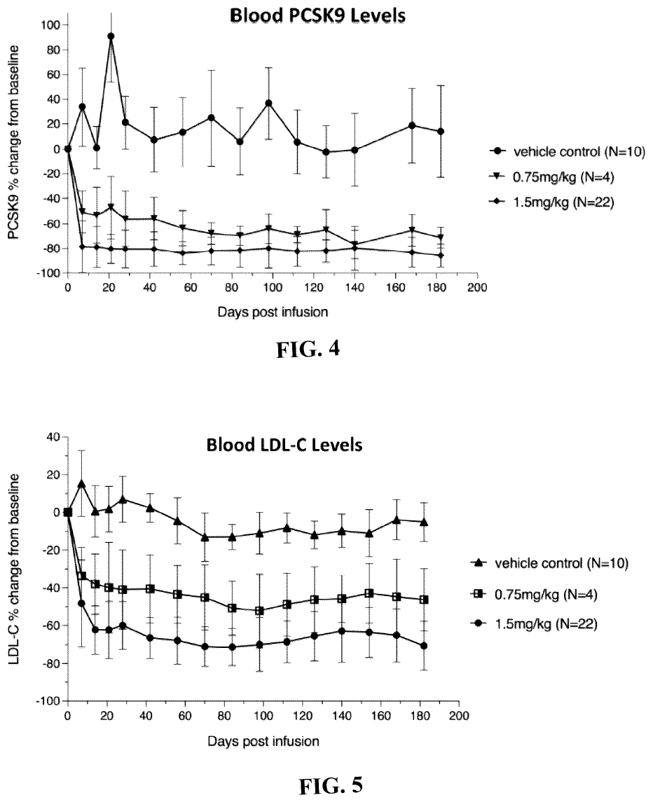

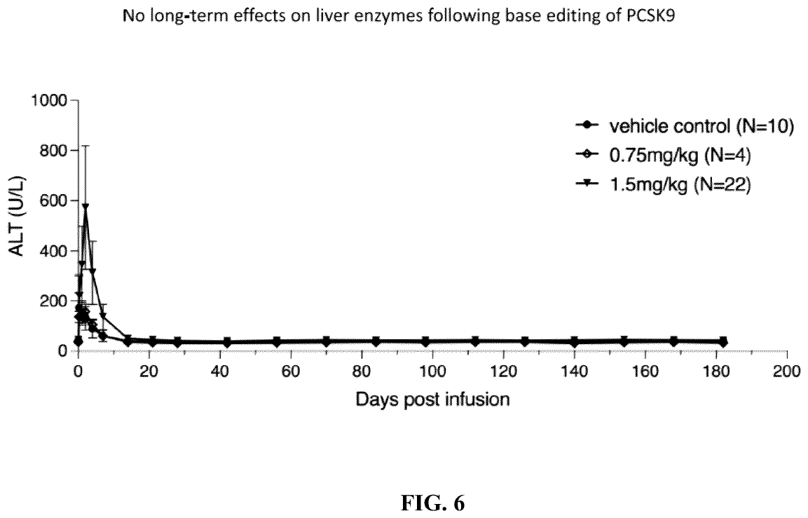

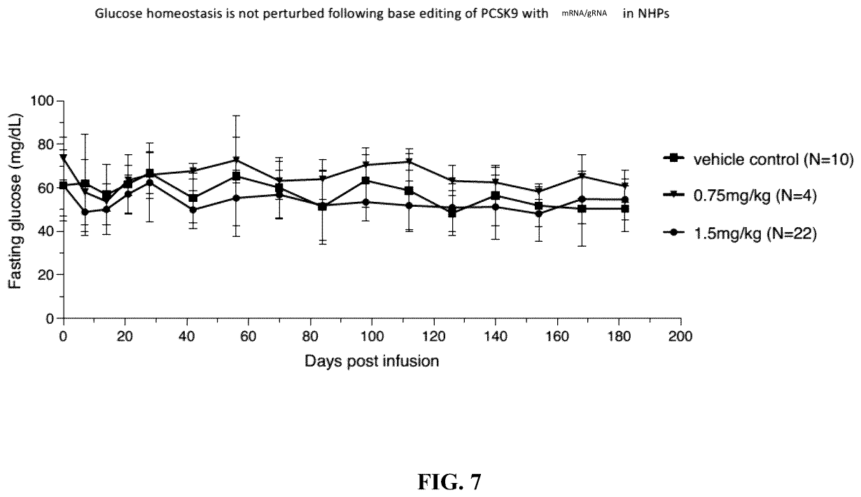

Illustrated in FIGS. 2-7 is a study protocol (FIG. 2) and data generated from the study setting forth PCSK9 editing percent change from baseline through liver biopsy assessing whole liver DNA editing at day 15 post infusion and PCSK9 protein reduction at day 14 post infusion (FIG. 3), PCSK9 protein reduction over an extended period of time post infusion (FIG. 4), LDL-C reduction over an extended period of time post infusion (FIG. 5), ALT enzyme levels over an extended period of time post infusion (FIG. 6), and fasting glucose levels over an extended time period post infusion (FIG. 7).

As illustrated in FIG. 2, the study involved a first group of 4 NHPs intravenously infused with a single dose of 0.75 mg/kg, a second group of 22 NHPs intravenously infused with a single dose of 1.5 mg/kg, and third group (“vehicle control”) of 10 NHPs intravenously infused with a single dose of the same LNP delivery vehicle without the gRNA and mRNA drug substance payload. The 0.75 mg/kg and 1.5 mg/kg doses were calculated using the total RNA drug substance payload (i.e., the combined weight of mRNA and gRNA) and does not include the weight of the lipid nanoparticle delivery vehicle. Thus, for example a dose of 0.75 mg/kg equates to 0.75 mg of combined mRNA and gRNA weight being intravenously infused in the NHP for each kilogram weight of the NHP being dosed. Each group of NHPs was dosed via a single intravenous infusion using the same LNP delivery vehicle, previously described herein. For groups 1 and 2, the formulated LNP comprised a payload of mRNA MA004 and guide RNA GA346 at a 1:1 mRNA:gRNA weight ratio. For the vehicle control group 3, the 10 NHPs were dosed with the same LNP as in groups 1 and 2, except that the LNP dosed did not contain any drug substance (i.e., no mRNA or gRNA). Blood serum samples from each NHP group were collected just prior to dosing and at the intervals noted in FIGS. 2-7 and analyzed for primary endpoints including: (1) blood PCSK9 levels, (2) Blood LDL-C levels as well as safety endpoints, including (1) liver function testing, (2) glucose homeostasis, and (3) adenine deaminase (ADAs) levels, which are used as an indicator of cytokine activity. Liver tissue samples, via liver biopsy, were collected from each group of NHPs at regular intervals and were analyzed for primary endpoints including whole liver PCSK9 DNA editing.

FIG. 3 is a waterfall plot of percent PCSK9 reduction in blood as compared to baseline at day 14 post infusion. The left side of the plot illustrates the 10 vehicle control group NHPs and the right side of the plot illustrates the 22 NHPs that were dosed with 1.5 mg/kg of drug substance. Each bar on the plot represents an individual NHP. As illustrated in the plot the mean PCSK9 reduction of the 22 NHPs in group was approximately 87% at 14 days post infusion. The high level of consistency in PCSK9 reduction is indicated in the plot across all 22 dosed animals as compared to the 10 vehicle treated NHP which PCSK9 blood levels scatter above and below pre-dosed baseline.

FIG. 4 is a graph representing the change in blood PCSK9 levels in the three groups of NHPs over an extended period (measured from post infusion) as a percentage change from baseline (indicated as 0 on the graph). Blood PCSK9 levels were analyzed from the blood serum collected from the NHPs at the intervals indicated in the graph of FIG. 4. As illustrated in FIG. 4, consistent and durable dose response at the two dose levels shown (0.75 mg/kg and 1.5 mg/kg) was observed and provides further confirmation to the study described in the 3 mg/kg dose NHP study described in PCT Application Publication NO. WO2021/207712, which is hereby incorporated by reference in its entirety.

FIG. 5 is a graph illustrating the change in blood LDL-C as a percentage change from pre-dosed baseline at the same intervals and over the same extended period of time post infusion as in FIG. 4. As illustrated in FIG. 5, blood LDL-C is down greater than 60% at the higher dose 1.5 mg/kg in the 22 NHPs of group 2 and reduced greater than 40% at the lower dose of 0.75 mg/kg of 4 NHPs in group 1. The vehicle control group 3 of 10 NHPs remained near post infusion baseline. Notably, the LDL-C reductions in group 1 and 2 were observed to be durability sustained over the test period thereby further supporting that a single dose of the drug substances described herein are capable of durably decreasing lifetime exposure to blood lipids (LDL-C), which are known to cause ASCVD, and thereby provides further confirmation of the described in connection with the study described in PCT Application Publication NO. WO2021/207712, which is hereby incorporated by reference in its entirety.

FIG. 6 is a graph illustrating Alanine Aminotransferase (ALT) levels of the three groups of NHPs in the study as compared to pre-dosed baseline at the same intervals and over the same extended period of time post infusion as in FIGS. 4-5. ALT is a liver enzyme that is commonly used as a marker to measure liver inflammation or damage and is here presented as measure of safety of the dosed drug product and substances described herein. As illustrated in FIG. 6, ALT levels, as measured against pre-dosed baseline rise immediately after dosing and then resolve to baseline levels by about day 14 post infusion and are maintained at pre-dosed baseline levels for the remainder of the extended study period, which provides further confirmation of the study described in connection with in PCT Application Publication NO. WO2021/207712, which is hereby incorporated by reference in its entirety, that no long-term effects on liver enzymes is observed following base editing of PCSK9 gene.

FIG. 7 is a graph illustrating fasting glucose levels of the three groups of NHPs in the study again taken at the same intervals and over the same extended period of time post infusion as in FIGS. 4-6. Glucose homeostasis was assessed via a glucose bioassay. Although prior therapies targeting PCSK9 have not shown impact on glucose homeostasis, given the differences between those prior therapies and the bae editing therapy modality that is the subject of this application, this analysis was undertaken to affirmatively rule out a theoretical risk of impacting glucose homeostasis by systemic PCSK9 inhibition. As illustrated in FIG. 7, glucose homeostasis was not perturbed following base editing of PCSK9 gene of the dosed NHPs. Further, although not illustrated in FIG. 7, a separate sampling of the study NHPs was conducted during ad libitum eating on several days and no impact on post-prandial glucose levels were observed.

### Example 3. Partial Hepatectomy Mouse Model to Assess Durability of PCSK9 Base Editor in Regenerated Liver Cells

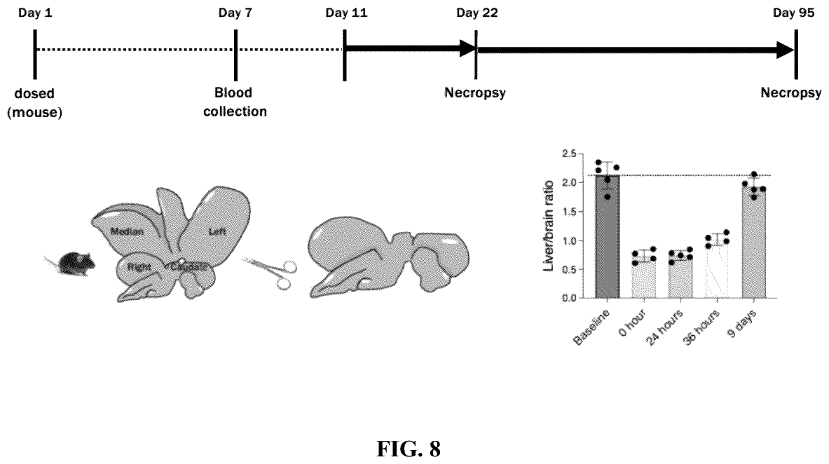

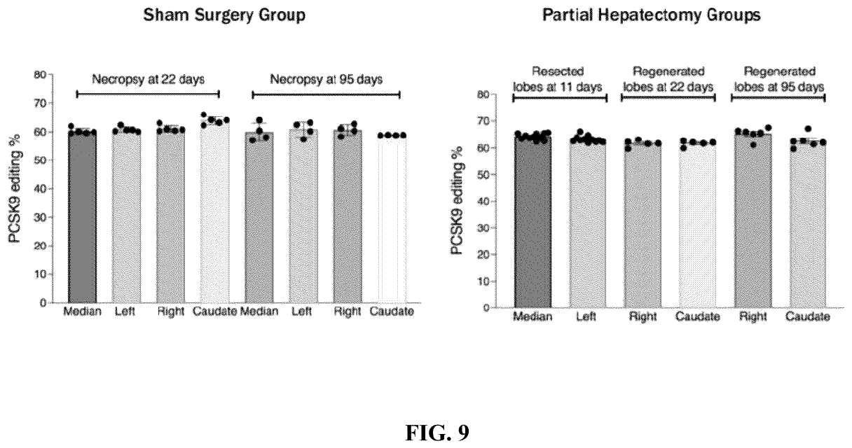

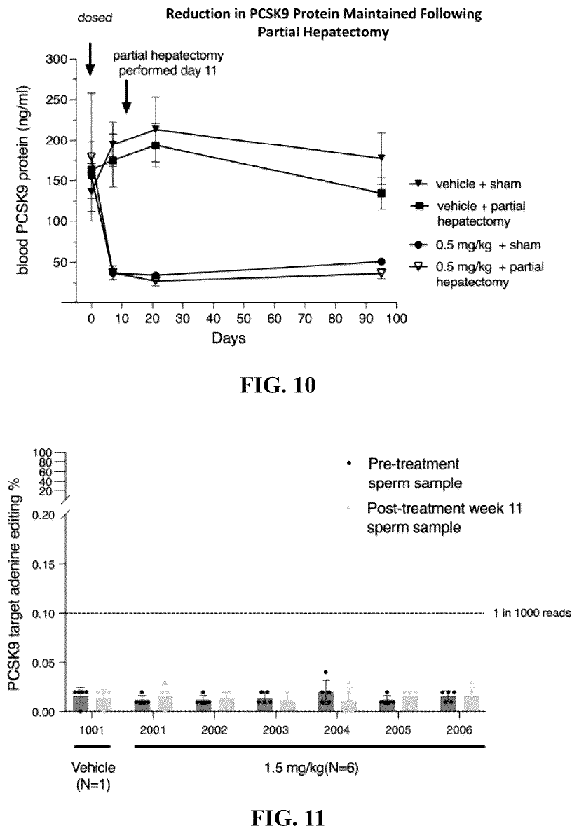

Illustrated in FIGS. 8-10 is a mouse study protocol (FIG. 8) and data generated from the study illustrating the durability of liver editing in regenerated PCSK9 edited liver cells (FIGS. 9-10). As illustrated in FIGS. 8-10, four groups of mice were dosed with a single intravenous infusion of the same LNP delivery vehicle described herein and in connection with Example 2. The first group of mice were dosed via intravenous infusion with 0.5 mg/kg, i.e., 0.5 mg of drug substance RNA (mRNA and guide RNA) at a 1:1 mRNA:guide RNA weight ratio) per kilogram weight of mouse and a partial hepatectomy was performed on day 11 post infusion of the mice in first group and resected lobes of the liver obtained from that surgery collected and analyzed for PCSK9 editing. The second group of mice were dosed via intravenous infusion with the same 0.5 mg/kg as the first group and a sham partial hepatectomy surgery was performed on day 11 post infusion of the mice in this second group. The third group of mice were dosed with the same LNP vehicle minus the mRNA MA004 and guide RNA GA257 payload and a partial hepatectomy surgery was performed on day 11 post infusion of the mice in this third group. A fourth group of mice were dosed with the same LNP vehicle minus the mRNA MA004 and guide RNA GA257 payload and a sham hepatectomy surgery was performed on day 11 post infusion of the mice in this fourth group. Blood serum was collected prior to dosing, 7 days post infusion, and at intervals identified in FIG. 10 and used to measure blood PCSK9 protein. On day 22 post infusion, a portion of each group of mice were sacrificed and the liver tissue in those mice was analyzed for PCSK9 editing. On day 95 post infusion, the remaining mice in the study were sacrificed and the liver tissue in those mice was analyzed for PCSK9 editing.

The lower, right graph of FIG. 8 depicts the time-dependent variation of the liver/brain ratio after partial hepatectomy in the mice that are the subject of the study. As indicated therein a nearly normal ratio of Liver/Brain in the mice was found to be re-established approximately 9 days after partial hepatectomy.

The group 1 mice are referred to in FIG. 10 as “0.5 mg/kg+partial hepatectomy.” The group 2 mice are referred to in FIG. 10 as “0.5 mg/kg+sham.” The group 3 mice are referred to in FIG. 10 as “vehicle+partial hepatectomy.” The group 4 mice are referred to in FIG. 10 as “vehicle+sham.” As illustrated in FIG. 1, reduction in blood PCSK9 measured from the collected blood serum samples was maintained following partial hepatectomy of group 1 and group 2 mice even after multiple liver regeneration cycles post partial hepatectomy.

Illustrated in FIG. 9 is PCSK9 editing data assessed via NGS (as previously described) from the liver samples taken from the resected lobes of the dosed mice that underwent partial hepatectomy and at necropsy of the mice at 22 and 95 day post infusion, As illustrated on the left graph of FIG. 9, the sham surgery group 2 mice that was dosed with 0.5 mg/kg maintained an PCSK9 editing percentage of around 60 percent in the mice sacrificed at both 22 and 95 days post infusion. As illustrated in right side graph of FIG. 9, the group 1 mice that was dosed with 0.5 mg/kg and underwent a partial hepatectomy at day 11 also maintained a PCSK9 editing percentage of around or greater than 60 percent and that PCSK9 editing percentage was comparable to the PCSK9 editing percentages assessed from the resected lobes retained from those mice from the partial hepatectomy surgery on day 11 post infusion. This study therefore confirms that base editing of PCSK9 using the dosed drug formulations described herein is sustained in regenerated liver lobes of treated subjects.

### Example 4. Confirmation of No Evidence of Editing Sperm in Sexually Mature Male NHPs

Illustrated in FIG. 11 is data from an NHP study assessing the level of PCSK9 editing in sperm of sexually mature male NHPs. In this study, 6 sexually mature male NHP were intravenously infused with the same single dose of 1.5 mg/kg that was used in the second group of 22 NHPs in Example 2. A seventh sexually mature male vehicle control monkey was intravenously dosed with the same LNP delivery vehicle without the gRNA GA346 and mRNA MA004 drug substance payload vehicle used in the third group (the “vehicle control”) of 10 NHPs in Example 2. Sperm samples were collected prior to dosing and 11 weeks after treatment which is greater than one full cycle of spermatogenesis. Quantitation of the levels of editing was done using a highly sensitive targeted amplicon assay optimized to have a high sensitivity. As illustrated in FIG. 11, no evidence of editing of the germline sperm was observed as the levels of editing quantitated in the sperm samples of the subject NHPs were similar between control and treated animals.

### Example 5. Off-Target Assessment

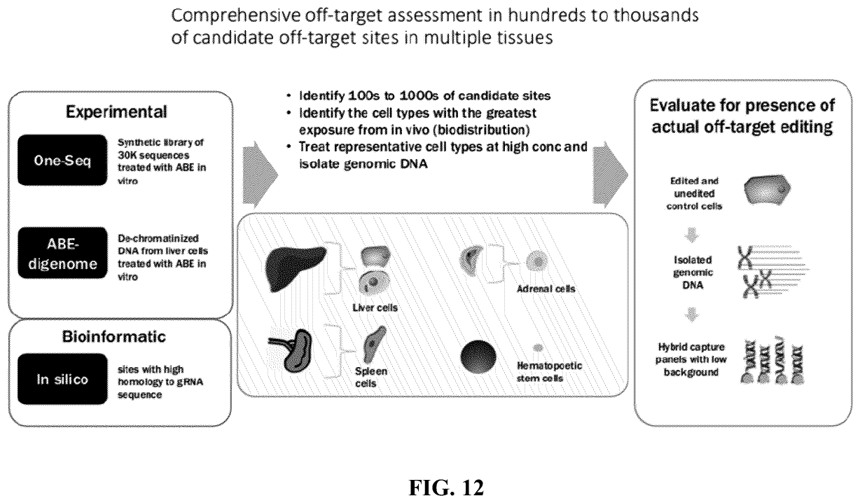

Illustrated in FIGS. 12-15 is a study protocol and data generated therefrom assessing potential off-target candidate sites of the PCSK9 base editor formulations described herein. FIG. 12 is a schematic illustrating the experimental methods used for evaluating the presence of actual off-target editing by the PCSK9 base editor formulations described herein. On the left is a description of the discovery methods to identify potential candidate off-targets sites using multiple techniques. As illustrated therein, the discovery assays comprise the One-Seq assay (Ref: https://www.biorxiv.org/content/10.1101/2021.04.05.438458v1) and an ABE-digenome assay. In One-Seq a synthetic library of tens of thousands of dsDNA molecules corresponding to sites of potential off-target activity based on homology are evaluated in vitro for cutting at a high dose of base editor protein. In ABE-digenome-seq, genomic DNA from primary liver cells are extracted and digested in vitro with ABE protein and potential sites of base modification are determined by a genome wide next gen sequencing analysis. Both of these assays yield sites that could be cleaved via off-target activity. To further guard against potential false negatives, a bioinformatic assessment is made of sites with a high degree of homology that were not identified in either One-Seq or ABE-Digenome discovery assay and these sites are also included in subsequent panel based validation assays.

Once the potential off-target sites are identified (which can number in the hundreds to thousands) with these discovery methods, an evaluation for the presence of actual off target editing was performed using a highly sensitive hybrid capture assay. This evaluation was performed in multiple primary cells from multiple donors and represent multiple tissues that could be exposed following systemic treatment with the PCSK9 base editor formulations described herein. These tissues include liver, spleen, adrenal cells, and hematopoietic stem cells, which is representative of a highly proliferative cell.

Using the foregoing, potential off-target activity panels were prepared, and hybrid capture methods were used to assay hundreds to thousands of sites for off-target activity. The sites corresponding to the panel were enriched by capture using RNA probes. The enriched DNA is then processed for sequencing and quantitation of base modification across all the panel sites.

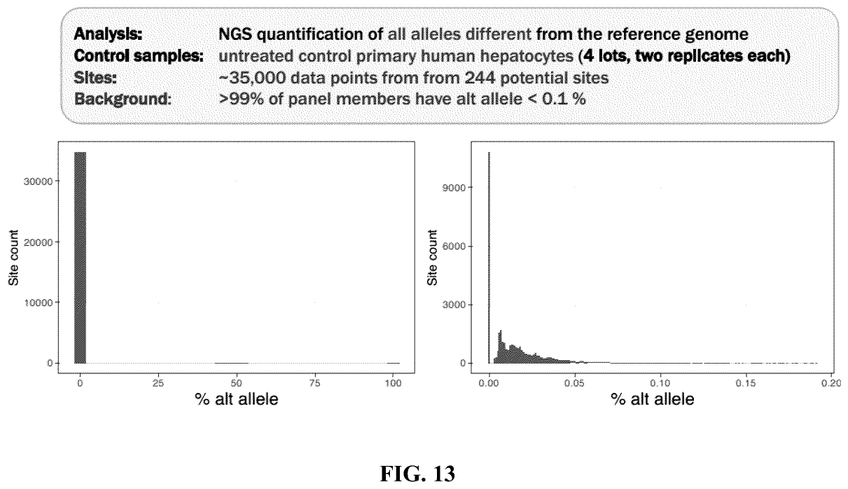

As illustrated in FIG. 13, the assays employed by this methodology result in a highly sensitive hybrid capture off target validation assay that has extremely low background with greater than 99% of the panel members having less than 0.1% of non-reference sequence (referred to as alt allele). The clean sensitivity of this hybrid capture assay as herein optimized for detecting off-target activity has allowed for profiling and detection of potential off-target candidate sites with a high level of sensitivity and is capable of scaling to accommodate thousands of sites.

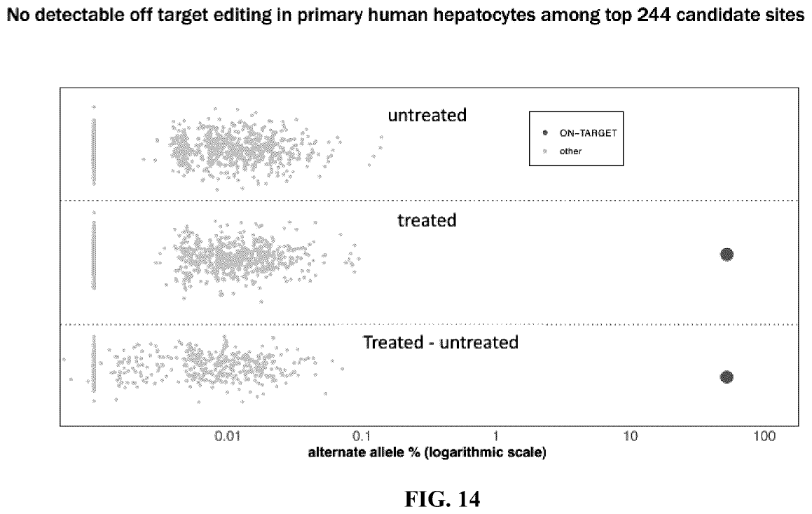

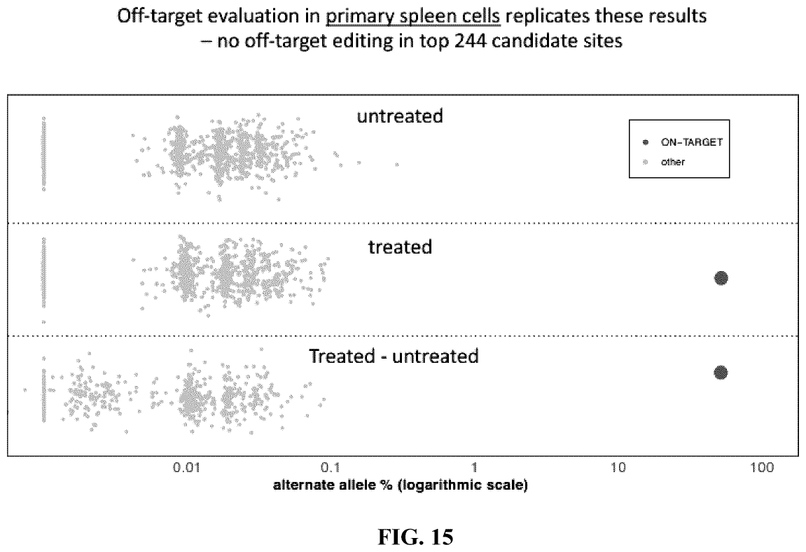

FIGS. 14-15 are jitter plots generated from the foregoing method that illustrate the quantitation of editing levels across base positions for a window of 10 bases around the target base for the top 244 candidate off-target sites in multiple cells. The jitter plot depicted in FIG. 14 represent assessment against primary human hepatocytes across a single PHH lot. As illustrated by comparison of the human liver cells treated with LNP formulation comprising a payload of mRNA MA004 and guide RNA GA346 as compared to untreated human livers, there is no detectable off-target editing in primary hepatocytes among any of the top 244 candidate sites. Similarly, the jitter plot depicted in FIG. 15 represents assessment against primary human spleen cells across a single lot of primary splenic endothelial cells. As illustrated by comparison between the primary human spleen cells transfected (or treated) with mRNA MA004 and guide RNA GA346 as compared to untreated primary human spleen cells, there is no detectable off-target editing observed in primary human spleen cells among any of the top 244 candidate sites.

### Example 6. Bio-Distribution of Editing of PCSK9

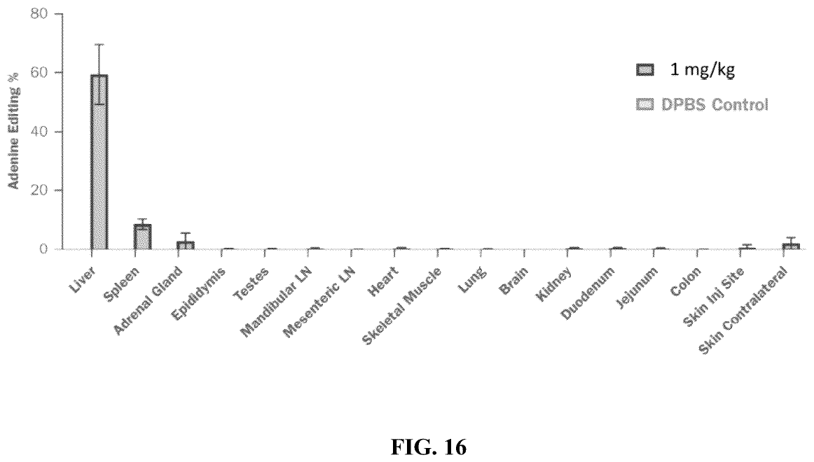

FIG. 16 represents data from a bio-distribution study of NHPs dosed with an LNP constituted with an ABE mRNA MA004 and guide RNA GA346. A single 1 mg/kg dose (1:1 mRNA:gRNA weight ratio) was administered via intravenous infusion to 3 NHPs with two control NHP being administered a Dulbecco phosphate buffered saline on the same day. All the NHPs (dosed and control) were sacrificed on day 15 post infusion and tissue samples of the sacrificed NHPs were collected and analyzed for PCSK9 editing.

Percent adenine editing of the PCSK9 gene in each tissue sample was assessed using NGS and the average adenine editing percentage is set forth in the bar graph in FIG. 16. PCSK9 editing was predominately distributed to the liver.

### Example 7. ABE Variants

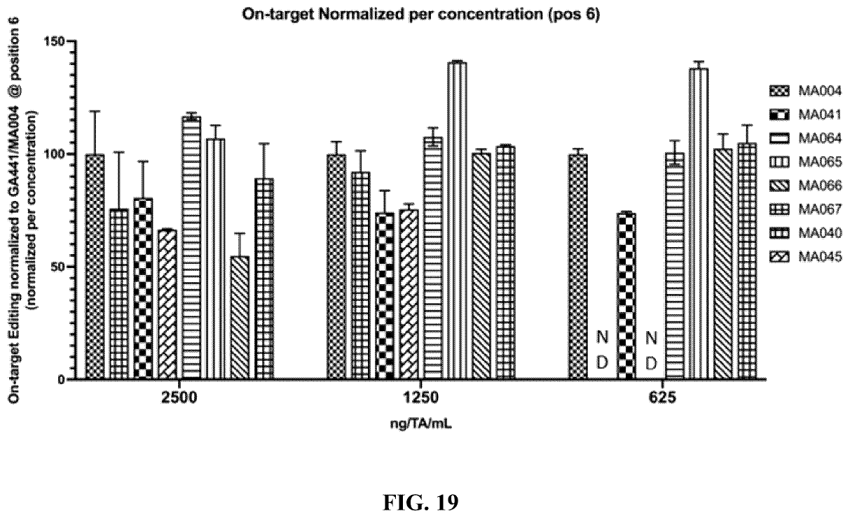

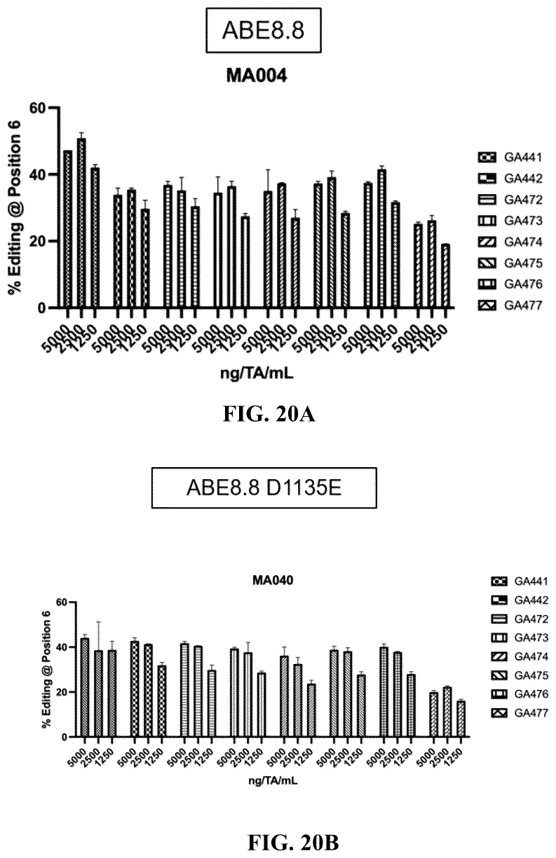

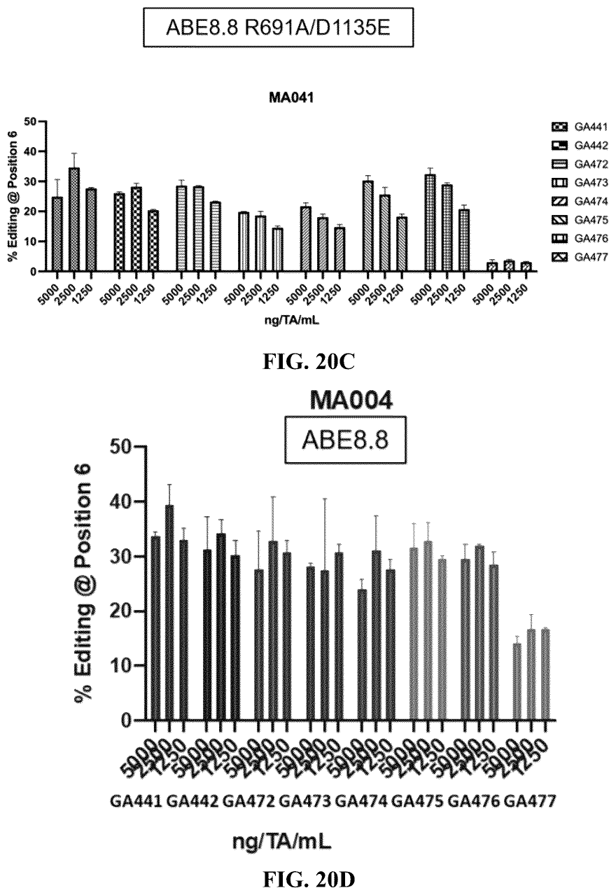

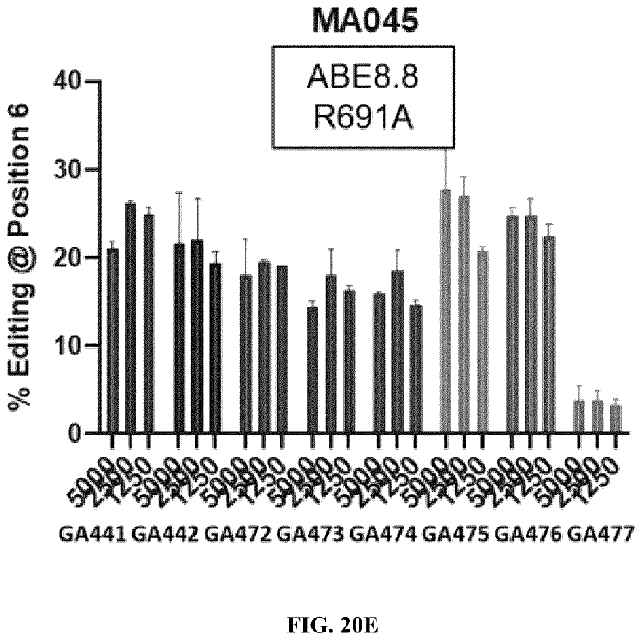

Exemplary modified ABE mRNA variants and sequences are described herein, including as specified in Tables 1 and 2. The on-target editing efficiency of the exemplary modified ABE variants in combination with guide RNAs targeting ANGPTL3 are shown in FIGS. 19 and 20A-20E. The editing of the off-target site candidates B2 and B6 of the exemplary modified ABE variants in combination with guide RNAs targeting ANGPTL3 are shown in FIGS. 26 and 27. The editing experiments were performed similarly as described in previous examples.

TABLE 1
Summary of sequence differences between MA040, MA041, MA045
MA064 MA065, MA066, and MA067

| | Amino acid | |
|---|---|---|
| | mutations | |
| | in SpCas9 | Codon differences in adenine base |
| | nickase | editor coding region vs MA004 |
| ID | region | (bolded and underlined) |
| MA040 | SpCas9 | SpCas9 nickase location: |
| | nickase | Codon 1135 is |
| | location: | changed from |
| | D1135E | GAC to GAG |
| | ABE8.8 | ABE8.8 location: |
| | location: | Codon 1333 is changed from |
| | D1333E | GAC to GAG |
| MA041 | SpCas9 | SpCas9 nickase location: |
| | nickase | Codon 691 is changed from |
| | location: | CGG to GCC |
| | R691A, | Codon 1135 is changed from |
| | D1135E | GAC to GAG |
| | ABE8.8 | ABE8.8 location: |
| | location: | Codon 889 is changed from CGG to GCC |
| | R889A, | Codon 1333 is changed from |
| | D1333E | GAC to GAG |
| MA045 | SpCas9 | SpCas9 nickase |
| | nickase | location: |
| | location: | Codon 691 is changed from |
| | R691A | CGG to GCC |
| | ABE8.8 | ABE8.8 location: |
| | location: | Codon 889 is changed from |
| | R889A | CGG to GCC |
| MA064 | SpCas9 | SpCas9 nickase location: |
| Hypa | nickase | Codon 692 is changed from |
| | location: | AAC to GCC |
| | N692A, | Codon 694 is changed from AUG to GCC |
| | M694A, | Codon 695 is changed from CAG to GCC |
| | Q695A, | Codon 698 is changed from |
| | H698A | CAC to GCC |
| | ABE8.8 | ABE8.8 location: |
| | location: | Codon 890 is changed from AAC to GCC |
| | N890A, | Codon 892 is changed from AUG to GCC |
| | M892A, | Codon 893 is changed from CAG to GCC |
| | Q893A, | Codon 896 is changed from |
| | H896A | CAC to GCC |
| MA065 | SpCas9 | SpCas9 nickase location: |
| | nickase | Codon 855 is changed from |
| | location: | AAG to GCC |
| | K855A | ABE8.8 location: |
| | ABE8.8 | Codon 1053 is |
| | location: | changed from |
| | K1053A | AAG to GCC |
| MA066 | SpCas9 | SpCas9 nickase location: |
| eSpCas9(1.1) | nickase | Codon 848 is changed from |
| | location: | AAG to GCC |
| | K848A, | Codon 1003 is changed from AAG to GCC |
| | K1003A, | Codon 1060 is changed from |
| | R1060A | CGG to GCC |
| | ABE8.8 | ABE8.8 location: |
| | location: | Codon 1046 is changed from AAG to GCC |
| | K1046A, | Codon 1201 is changed from AAG to GCC |
| | K1201A, | Codon 1258 is changed from |
| | K1258A | CGG to GCC |
| MA067 | SpCas9 | SpCas9 nickase location: |
| eSpCas9(1.0) | nickase | Codon 810 is changed from AAG to GCC |
| | location: | Codon 1003 is changed from |
| | K810A, | AAG to GCC |
| | K1003A, | Codon 1060 is changed from |
| | R1060A | CGG to GCC |
| | ABE8.8 | ABE8.8 location: |
| | location: | Codon 1008 is changed from AAG to GCC |
| | K1008A, | Codon 1201 is changed from AAG to GCC |
| | K1201A, | Codon 1258 is changed from |
| | K1258A | CGG to GCC |

TABLE 2
Sequences for high fidelity ABEs MA040, MA041, MA045, MA064 MA065,
MA066, and MA067
MA040 mRNA sequence:

| | | SEQ ID |
|---|---|---|
| Region | Sequence | NO |
| 5′ | AGGAAAu′AAGAGAGAAAAGAAGAGu′AAGAAGAAAu′Au′AA | 689 |
| UTR | GAGCCACC | |
| High | ATGAGCGAGGTGGAGTTCAGCCACGAGTACTGGATGCGGCAC | 717 |
| fidelity | GCCCTGACCCTGGCCAAGCGGGCCCGGGACGAGCGGGAGGT | |
| ABE | GCCCGTGGGCGCCGTGCTGGTGCTGAACAACCGGGTGATCGG | |
| CDS | CGAGGGCTGGAACCGGGCCATCGGCCTGCACGACCCCACCGC | |
| | CCACGCCGAGATCATGGCCCTGCGGCAGGGCGGCCTGGTGAT | |
| | GCAGAACTACCGGCTGATCGACGCCACCCTGTACGTGACCTT | |
| | CGAGCCCTGCGTGATGTGCGCCGGCGCCATGATCCACAGCCG | |
| | GATCGGCCGGGTGGTGTTCGGCGTGCGGAACGCCAAGACCGG | |
| | CGCCGCCGGCAGCCTGATGGACGTGCTGCACCACCCCGGCAT | |
| | GAACCACCGGGTGGAGATCACCGAGGGCATCCTGGCCGACG | |
| | AGTGCGCCGCCCTGCTGTGCCGGTTCTTCCGGATGCCCCGGCG | |
| | GGTGTTCAACGCCCAGAAGAAGGCCCAGAGCAGCACCGACA | |
| | GCGGCGGCAGCAGCGGCGGCAGCAGCGGCAGCGAGACACCC | |
| | GGCACCAGCGAGAGCGCCACCCCCGAGAGCAGCGGCGGCAG | |
| | CAGCGGCGGCAGCGACAAGAAGTACAGCATCGGCCTGGCCA | |
| | TCGGCACCAACAGCGTGGGCTGGGCCGTGATCACCGACGAGT | |
| | ACAAGGTGCCCAGCAAGAAGTTCAAGGTGCTGGGCAACACC | |
| | GACCGGCACAGCATCAAGAAGAACCTGATCGGCGCCCTGCTG | |
| | TTCGACAGCGGCGAGACAGCCGAGGCCACCCGGCTGAAGCG | |
| | GACCGCCCGGCGGCGGTACACCCGGCGGAAGAACCGGATCT | |
| | GCTACCTGCAGGAGATCTTCAGCAACGAGATGGCCAAGGTGG | |
| | ACGACAGCTTCTTCCACCGGCTGGAGGAGAGCTTCCTGGTGG | |
| | AGGAGGACAAGAAGCACGAGCGGCACCCCATCTTCGGCAAC | |
| | ATCGTGGACGAGGTGGCCTACCACGAGAAGTACCCCACCATC | |
| | TACCACCTGCGGAAGAAGCTGGTGGACAGCACCGACAAGGC | |
| | CGACCTGCGGCTGATCTACCTGGCCCTGGCCCACATGATCAA | |
| | GTTCCGGGGCCACTTCCTGATCGAGGGCGACCTGAACCCCGA | |
| | CAACAGCGACGTGGACAAGCTGTTCATCCAGCTGGTGCAGAC | |
| | CTACAACCAGCTGTTCGAGGAGAACCCCATCAACGCCAGCGG | |
| | CGTGGACGCCAAGGCCATCCTGAGCGCCCGGCTGAGCAAGAG | |
| | CCGGCGGCTGGAGAACCTGATCGCCCAGCTGCCCGGCGAGAA | |
| | GAAGAACGGCCTGTTCGGCAACCTGATCGCCCTGAGCCTGGG | |
| | CCTGACCCCCAACTTCAAGAGCAACTTCGACCTGGCCGAGGA | |
| | CGCCAAGCTGCAGCTGAGCAAGGACACCTACGACGACGACCT | |
| | GGACAACCTGCTGGCCCAGATCGGCGACCAGTACGCCGACCT | |
| | GTTCCTGGCCGCCAAGAACCTGAGCGACGCCATCCTGCTGAG | |
| | CGACATCCTGCGGGTGAACACCGAGATCACCAAGGCCCCCCT | |
| | GAGCGCCAGCATGATCAAGCGGTACGACGAGCACCACCAGG | |
| | ACCTGACCCTGCTGAAGGCCCTGGTGCGGCAGCAGCTGCCCG | |
| | AGAAGTACAAGGAGATCTTCTTCGACCAGAGCAAGAACGGCT | |
| | ACGCCGGCTACATCGACGGCGGCGCCAGCCAGGAGGAGTTCT | |
| | ACAAGTTCATCAAGCCCATCCTGGAGAAGATGGACGGCACCG | |
| | AGGAGCTGCTGGTGAAGCTGAACCGGGAGGACCTGCTGCGG | |
| | AAGCAGCGGACCTTCGACAACGGCAGCATCCCCCACCAGATC | |
| | CACCTGGGCGAGCTGCACGCCATCCTGCGGCGGCAGGAGGAC | |
| | TTCTACCCCTTCCTGAAGGACAACCGGGAGAAGATCGAGAAG | |
| | ATCCTGACCTTCCGGATCCCCTACTACGTGGGCCCCCTGGCCC | |
| | GGGGCAACAGCCGGTTCGCCTGGATGACCCGCAAGAGCGAG | |
| | GAGACAATCACCCCCTGGAACTTCGAGGAGGTGGTGGACAAG | |
| | GGCGCCAGCGCCCAGAGCTTCATCGAGCGGATGACCAACTTC | |
| | GACAAGAACCTGCCCAACGAGAAGGTGCTGCCCAAGCACAG | |
| | CCTGCTGTACGAGTACTTCACCGTGTACAACGAGCTGACCAA | |
| | GGTGAAGTACGTGACCGAGGGCATGCGGAAGCCCGCCTTCCT | |
| | GAGCGGCGAGCAGAAGAAGGCCATCGTGGACCTGCTGTTCAA | |
| | GACCAACCGGAAGGTGACCGTGAAGCAGCTGAAGGAGGACT | |
| | ACTTCAAGAAGATCGAGTGCTTCGACAGCGTGGAGATCAGCG | |
| | GCGTGGAGGACCGGTTCAACGCCAGCCTGGGCACCTACCACG | |
| | ACCTGCTGAAGATCATCAAGGACAAGGACTTCCTGGACAACG | |
| | AGGAGAACGAGGACATCCTGGAGGACATCGTGCTGACCCTGA | |
| | CCCTGTTCGAGGACCGGGAGATGATCGAGGAGCGGCTGAAG | |
| | ACCTACGCCCACCTGTTCGACGACAAGGTGATGAAGCAGCTG | |
| | AAGCGGCGGCGGTACACCGGCTGGGGCCGGCTGAGCCGGAA | |
| | GCTGATCAACGGCATCCGGGACAAGCAGAGCGGCAAGACCA | |
| | TCCTGGACTTCCTCAAGAGCGACGGCTTCGCCAACCGGAACT | |
| | TCATGCAGCTGATCCACGACGACAGCCTGACCTTCAAGGAGG | |
| | ACATCCAGAAGGCCCAGGTGAGCGGCCAGGGCGACAGCCTG | |
| | CACGAGCACATCGCCAACCTGGCCGGCAGCCCCGCCATCAAG | |
| | AAGGGCATCCTGCAGACCGTGAAGGTGGTGGACGAGCTGGTG | |
| | AAGGTGATGGGCCGGCACAAGCCCGAGAACATCGTGATCGA | |
| | GATGGCCCGGGAGAACCAGACCACCCAGAAGGGCCAGAAGA | |
| | ACAGCCGGGAGCGGATGAAGCGGATCGAGGAGGGCATCAAG | |
| | GAGCTGGGCAGCCAGATCCTGAAGGAGCACCCCGTGGAGAA | |
| | CACCCAGCTGCAGAACGAGAAGCTGTACCTGTACTACCTGCA | |
| | GAACGGCCGGGACATGTACGTGGACCAGGAGCTGGACATCA | |
| | ACCGGCTGAGCGACTACGACGTGGACCACATCGTGCCCCAGA | |
| | GCTTCCTGAAGGACGACAGCATCGACAACAAGGTGCTGACCC | |
| | GGAGCGACAAGAACCGGGGCAAGAGCGACAACGTGCCCAGC | |
| | GAGGAGGTGGTGAAGAAGATGAAGAACTACTGGCGGCAGCT | |
| | GCTGAACGCCAAGCTGATCACCCAGCGGAAGTTCGACAACCT | |
| | GACCAAGGCCGAGCGGGGCGGCCTGAGCGAGCTGGACAAGG | |
| | CCGGCTTCATCAAGCGGCAGCTGGTGGAGACACGGCAGATCA | |
| | CCAAGCACGTGGCCCAGATCCTGGACAGCCGGATGAACACCA | |
| | AGTACGACGAGAACGACAAGCTGATCCGGGAGGTGAAGGTG | |
| | ATCACCCTCAAGAGCAAGCTGGTGAGCGACTTCCGGAAGGAC | |
| | TTCCAGTTCTACAAGGTGCGGGAGATCAACAACTACCACCAC | |
| | GCCCACGACGCCTACCTGAACGCCGTGGTGGGCACCGCCCTG | |
| | ATCAAGAAGTACCCCAAGCTGGAGAGCGAGTTCGTGTACGGC | |
| | GACTACAAGGTGTACGACGTGCGGAAGATGATCGCCAAGAG | |
| | CGAGCAGGAGATCGGCAAGGCCACCGCCAAGTACTTCTTCTA | |
| | CAGCAACATCATGAACTTCTTCAAGACCGAGATCACCCTGGC | |
| | CAACGGCGAGATCCGGAAGCGGCCCCTGATCGAGACAAACG | |
| | GCGAGACAGGCGAGATCGTGTGGGACAAGGGCCGGGACTTC | |
| | GCCACCGTGCGGAAGGTGCTGAGCATGCCCCAGGTGAACATC | |
| | GTGAAGAAGACCGAGGTGCAGACCGGCGGCTTCAGCAAGGA | |
| | GAGCATCCTGCCCAAGCGGAACAGCGACAAGCTGATCGCCCG | |
| | GAAGAAGGACTGGGACCCCAAGAAGTACGGCGGCTTCGAGA | |
| | GCCCCACCGTGGCCTACAGCGTGCTGGTGGTGGCCAAGGTGG | |
| | AGAAGGGCAAGAGCAAGAAGCTCAAGAGCGTGAAGGAGCTG | |
| | CTGGGCATCACCATCATGGAGCGGAGCAGCTTCGAGAAGAAC | |
| | CCCATCGACTTCCTGGAGGCCAAGGGCTACAAGGAGGTGAAG | |
| | AAGGACCTGATCATCAAGCTGCCCAAGTACAGCCTGTTCGAG | |
| | CTGGAGAACGGCCGGAAGCGGATGCTGGCCAGCGCCGGCGA | |
| | GCTGCAGAAGGGCAACGAGCTGGCCCTGCCCAGCAAGTACGT | |
| | GAACTTCCTGTACCTGGCCAGCCACTACGAGAAGCTGAAGGG | |
| | CAGCCCCGAGGACAACGAGCAGAAGCAGCTGTTCGTGGAGC | |
| | AGCACAAGCACTACCTGGACGAGATCATCGAGCAGATCAGCG | |
| | AGTTCAGCAAGCGGGTGATCCTGGCCGACGCCAACCTGGACA | |
| | AGGTGCTGAGCGCCTACAACAAGCACCGGGACAAGCCCATCC | |
| | GGGAGCAGGCCGAGAACATCATCCACCTGTTCACCCTGACCA | |
| | ACCTGGGCGCCCCCGCCGCCTTCAAGTACTTCGACACCACCA | |
| | TCGACCGGAAGCGGTACACCAGCACCAAGGAGGTGCTGGAC | |
| | GCCACCCTGATCCACCAGAGCATCACCGGCCTGTACGAGACA | |
| | CGGATCGACCTGAGCCAGCTGGGCGGCGACGAGGGCGCCGA | |
| | CAAGCGGACCGCCGACGGCAGCGAGTTCGAGAGCCCCAAGA | |
| | AGAAGCGGAAGGTGTGA | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| MA040 | MSEVEFSHEYWMRHALTLAKRARDEREVPVGAVLVLNNRVIGE | 718 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANR | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEKLYLYYLQNGRDM | |
| | YVDQELDINRLSDYDVDHIVPQSFLKDDSIDNKVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPKLESEFVYGDYKVYDVRKMIAKSEQEIGKATAKYFFYS | |
| | NIMNFFKTEITLANGEIRKRPLIETNGETGEIVWDKGRDFATVRK | |
| | VLSMPQVNIVKKTEVQTGGFSKESILPKRNSDKLIARKKDWDPK | |
| | KYGGFESPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

MA041 mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | ATGAGCGAGGTGGAGTTCAGCCACGAGTACTGGATGCGGCAC | 719 |
| fidelity | GCCCTGACCCTGGCCAAGCGGGCCCGGGACGAGCGGGAGGT | |
| ABE | GCCCGTGGGCGCCGTGCTGGTGCTGAACAACCGGGTGATCGG | |
| CDS | CGAGGGCTGGAACCGGGCCATCGGCCTGCACGACCCCACCGC | |
| | CCACGCCGAGATCATGGCCCTGCGGCAGGGCGGCCTGGTGAT | |
| | GCAGAACTACCGGCTGATCGACGCCACCCTGTACGTGACCTT | |
| | CGAGCCCTGCGTGATGTGCGCCGGCGCCATGATCCACAGCCG | |
| | GATCGGCCGGGTGGTGTTCGGCGTGCGGAACGCCAAGACCGG | |
| | CGCCGCCGGCAGCCTGATGGACGTGCTGCACCACCCCGGCAT | |
| | GAACCACCGGGTGGAGATCACCGAGGGCATCCTGGCCGACG | |
| | AGTGCGCCGCCCTGCTGTGCCGGTTCTTCCGGATGCCCCGGCG | |
| | GGTGTTCAACGCCCAGAAGAAGGCCCAGAGCAGCACCGACA | |
| | GCGGCGGCAGCAGCGGCGGCAGCAGCGGCAGCGAGACACCC | |
| | GGCACCAGCGAGAGCGCCACCCCCGAGAGCAGCGGCGGCAG | |
| | CAGCGGCGGCAGCGACAAGAAGTACAGCATCGGCCTGGCCA | |
| | TCGGCACCAACAGCGTGGGCTGGGCCGTGATCACCGACGAGT | |
| | ACAAGGTGCCCAGCAAGAAGTTCAAGGTGCTGGGCAACACC | |
| | GACCGGCACAGCATCAAGAAGAACCTGATCGGCGCCCTGCTG | |
| | TTCGACAGCGGCGAGACAGCCGAGGCCACCCGGCTGAAGCG | |
| | GACCGCCCGGCGGCGGTACACCCGGCGGAAGAACCGGATCT | |
| | GCTACCTGCAGGAGATCTTCAGCAACGAGATGGCCAAGGTGG | |
| | ACGACAGCTTCTTCCACCGGCTGGAGGAGAGCTTCCTGGTGG | |
| | AGGAGGACAAGAAGCACGAGCGGCACCCCATCTTCGGCAAC | |
| | ATCGTGGACGAGGTGGCCTACCACGAGAAGTACCCCACCATC | |
| | TACCACCTGCGGAAGAAGCTGGTGGACAGCACCGACAAGGC | |
| | CGACCTGCGGCTGATCTACCTGGCCCTGGCCCACATGATCAA | |
| | GTTCCGGGGCCACTTCCTGATCGAGGGCGACCTGAACCCCGA | |
| | CAACAGCGACGTGGACAAGCTGTTCATCCAGCTGGTGCAGAC | |
| | CTACAACCAGCTGTTCGAGGAGAACCCCATCAACGCCAGCGG | |
| | CGTGGACGCCAAGGCCATCCTGAGCGCCCGGCTGAGCAAGAG | |
| | CCGGCGGCTGGAGAACCTGATCGCCCAGCTGCCCGGCGAGAA | |
| | GAAGAACGGCCTGTTCGGCAACCTGATCGCCCTGAGCCTGGG | |
| | CCTGACCCCCAACTTCAAGAGCAACTTCGACCTGGCCGAGGA | |
| | CGCCAAGCTGCAGCTGAGCAAGGACACCTACGACGACGACCT | |
| | GGACAACCTGCTGGCCCAGATCGGCGACCAGTACGCCGACCT | |
| | GTTCCTGGCCGCCAAGAACCTGAGCGACGCCATCCTGCTGAG | |
| | CGACATCCTGCGGGTGAACACCGAGATCACCAAGGCCCCCCT | |
| | GAGCGCCAGCATGATCAAGCGGTACGACGAGCACCACCAGG | |
| | ACCTGACCCTGCTGAAGGCCCTGGTGCGGCAGCAGCTGCCCG | |
| | AGAAGTACAAGGAGATCTTCTTCGACCAGAGCAAGAACGGCT | |
| | ACGCCGGCTACATCGACGGCGGCGCCAGCCAGGAGGAGTTCT | |
| | ACAAGTTCATCAAGCCCATCCTGGAGAAGATGGACGGCACCG | |
| | AGGAGCTGCTGGTGAAGCTGAACCGGGAGGACCTGCTGCGG | |
| | AAGCAGCGGACCTTCGACAACGGCAGCATCCCCCACCAGATC | |
| | CACCTGGGCGAGCTGCACGCCATCCTGCGGCGGCAGGAGGAC | |
| | TTCTACCCCTTCCTGAAGGACAACCGGGAGAAGATCGAGAAG | |
| | ATCCTGACCTTCCGGATCCCCTACTACGTGGGCCCCCTGGCCC | |
| | GGGGCAACAGCCGGTTCGCCTGGATGACCCGCAAGAGCGAG | |
| | GAGACAATCACCCCCTGGAACTTCGAGGAGGTGGTGGACAAG | |
| | GGCGCCAGCGCCCAGAGCTTCATCGAGCGGATGACCAACTTC | |
| | GACAAGAACCTGCCCAACGAGAAGGTGCTGCCCAAGCACAG | |
| | CCTGCTGTACGAGTACTTCACCGTGTACAACGAGCTGACCAA | |
| | GGTGAAGTACGTGACCGAGGGCATGCGGAAGCCCGCCTTCCT | |
| | GAGCGGCGAGCAGAAGAAGGCCATCGTGGACCTGCTGTTCAA | |
| | GACCAACCGGAAGGTGACCGTGAAGCAGCTGAAGGAGGACT | |
| | ACTTCAAGAAGATCGAGTGCTTCGACAGCGTGGAGATCAGCG | |
| | GCGTGGAGGACCGGTTCAACGCCAGCCTGGGCACCTACCACG | |
| | ACCTGCTGAAGATCATCAAGGACAAGGACTTCCTGGACAACG | |
| | AGGAGAACGAGGACATCCTGGAGGACATCGTGCTGACCCTGA | |
| | CCCTGTTCGAGGACCGGGAGATGATCGAGGAGCGGCTGAAG | |
| | ACCTACGCCCACCTGTTCGACGACAAGGTGATGAAGCAGCTG | |
| | AAGCGGCGGCGGTACACCGGCTGGGGCCGGCTGAGCCGGAA | |
| | GCTGATCAACGGCATCCGGGACAAGCAGAGCGGCAAGACCA | |
| | TCCTGGACTTCCTCAAGAGCGACGGCTTCGCCAACGCCAACT | |
| | TCATGCAGCTGATCCACGACGACAGCCTGACCTTCAAGGAGG | |
| | ACATCCAGAAGGCCCAGGTGAGCGGCCAGGGCGACAGCCTG | |
| | CACGAGCACATCGCCAACCTGGCCGGCAGCCCCGCCATCAAG | |
| | AAGGGCATCCTGCAGACCGTGAAGGTGGTGGACGAGCTGGTG | |
| | AAGGTGATGGGCCGGCACAAGCCCGAGAACATCGTGATCGA | |
| | GATGGCCCGGGAGAACCAGACCACCCAGAAGGGCCAGAAGA | |
| | ACAGCCGGGAGCGGATGAAGCGGATCGAGGAGGGCATCAAG | |
| | GAGCTGGGCAGCCAGATCCTGAAGGAGCACCCCGTGGAGAA | |
| | CACCCAGCTGCAGAACGAGAAGCTGTACCTGTACTACCTGCA | |
| | GAACGGCCGGGACATGTACGTGGACCAGGAGCTGGACATCA | |
| | ACCGGCTGAGCGACTACGACGTGGACCACATCGTGCCCCAGA | |
| | GCTTCCTGAAGGACGACAGCATCGACAACAAGGTGCTGACCC | |
| | GGAGCGACAAGAACCGGGGCAAGAGCGACAACGTGCCCAGC | |
| | GAGGAGGTGGTGAAGAAGATGAAGAACTACTGGCGGCAGCT | |
| | GCTGAACGCCAAGCTGATCACCCAGCGGAAGTTCGACAACCT | |
| | GACCAAGGCCGAGCGGGGGGCCTGAGCGAGCTGGACAAGG | |
| | CCGGCTTCATCAAGCGGCAGCTGGTGGAGACACGGCAGATCA | |
| | CCAAGCACGTGGCCCAGATCCTGGACAGCCGGATGAACACCA | |
| | AGTACGACGAGAACGACAAGCTGATCCGGGAGGTGAAGGTG | |
| | ATCACCCTCAAGAGCAAGCTGGTGAGCGACTTCCGGAAGGAC | |
| | TTCCAGTTCTACAAGGTGCGGGAGATCAACAACTACCACCAC | |
| | GCCCACGACGCCTACCTGAACGCCGTGGTGGGCACCGCCCTG | |
| | ATCAAGAAGTACCCCAAGCTGGAGAGCGAGTTCGTGTACGGC | |
| | GACTACAAGGTGTACGACGTGCGGAAGATGATCGCCAAGAG | |
| | CGAGCAGGAGATCGGCAAGGCCACCGCCAAGTACTTCTTCTA | |
| | CAGCAACATCATGAACTTCTTCAAGACCGAGATCACCCTGGC | |
| | CAACGGCGAGATCCGGAAGCGGCCCCTGATCGAGACAAACG | |
| | GCGAGACAGGCGAGATCGTGTGGGACAAGGGCCGGGACTTC | |
| | GCCACCGTGCGGAAGGTGCTGAGCATGCCCCAGGTGAACATC | |
| | GTGAAGAAGACCGAGGTGCAGACCGGCGGCTTCAGCAAGGA | |
| | GAGCATCCTGCCCAAGCGGAACAGCGACAAGCTGATCGCCCG | |
| | GAAGAAGGACTGGGACCCCAAGAAGTACGGCGGCTTCGAGA | |
| | GCCCCACCGTGGCCTACAGCGTGCTGGTGGTGGCCAAGGTGG | |
| | AGAAGGGCAAGAGCAAGAAGCTCAAGAGCGTGAAGGAGCTG | |
| | CTGGGCATCACCATCATGGAGCGGAGCAGCTTCGAGAAGAAC | |
| | CCCATCGACTTCCTGGAGGCCAAGGGCTACAAGGAGGTGAAG | |
| | AAGGACCTGATCATCAAGCTGCCCAAGTACAGCCTGTTCGAG | |
| | CTGGAGAACGGCCGGAAGCGGATGCTGGCCAGCGCCGGCGA | |
| | GCTGCAGAAGGGCAACGAGCTGGCCCTGCCCAGCAAGTACGT | |
| | GAACTTCCTGTACCTGGCCAGCCACTACGAGAAGCTGAAGGG | |
| | CAGCCCCGAGGACAACGAGCAGAAGCAGCTGTTCGTGGAGC | |
| | AGCACAAGCACTACCTGGACGAGATCATCGAGCAGATCAGCG | |
| | AGTTCAGCAAGCGGGTGATCCTGGCCGACGCCAACCTGGACA | |
| | AGGTGCTGAGCGCCTACAACAAGCACCGGGACAAGCCCATCC | |
| | GGGAGCAGGCCGAGAACATCATCCACCTGTTCACCCTGACCA | |
| | ACCTGGGCGCCCCCGCCGCCTTCAAGTACTTCGACACCACCA | |
| | TCGACCGGAAGCGGTACACCAGCACCAAGGAGGTGCTGGAC | |
| | GCCACCCTGATCCACCAGAGCATCACCGGCCTGTACGAGACA | |
| | CGGATCGACCTGAGCCAGCTGGGCGGCGACGAGGGCGCCGA | |
| | CAAGCGGACCGCCGACGGCAGCGAGTTCGAGAGCCCCAAGA | |
| | AGAAGCGGAAGGTGTGA | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| MA041 | MSEVEFSHEYWMRHALTLAKRARDEREVPVGAVLVLNNRVIGE | 720 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANA | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEKLYLYYLQNGRDM | |
| | YVDQELDINRLSDYDVDHIVPQSFLKDDSIDNKVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPKLESEFVYGDYKVYDVRKMIAKSEQEIGKATAKYFFYS | |
| | NIMNFFKTEITLANGEIRKRPLIETNGETGEIVWDKGRDFATVRK | |
| | VLSMPQVNIVKKTEVQTGGFSKESILPKRNSDKLIARKKDWDPK | |
| | KYGGFESPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

MA045 mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | ATGAGCGAGGTGGAGTTCAGCCACGAGTACTGGATGCGGCAC | 721 |
| fidelity | GCCCTGACCCTGGCCAAGCGGGCCCGGGACGAGCGGGAGGT | |
| ABE | GCCCGTGGGCGCCGTGCTGGTGCTGAACAACCGGGTGATCGG | |
| CDS | CGAGGGCTGGAACCGGGCCATCGGCCTGCACGACCCCACCGC | |
| | CCACGCCGAGATCATGGCCCTGCGGCAGGGCGGCCTGGTGAT | |
| | GCAGAACTACCGGCTGATCGACGCCACCCTGTACGTGACCTT | |
| | CGAGCCCTGCGTGATGTGCGCCGGCGCCATGATCCACAGCCG | |
| | GATCGGCCGGGTGGTGTTCGGCGTGCGGAACGCCAAGACCGG | |
| | CGCCGCCGGCAGCCTGATGGACGTGCTGCACCACCCCGGCAT | |
| | GAACCACCGGGTGGAGATCACCGAGGGCATCCTGGCCGACG | |
| | AGTGCGCCGCCCTGCTGTGCCGGTTCTTCCGGATGCCCCGGCG | |
| | GGTGTTCAACGCCCAGAAGAAGGCCCAGAGCAGCACCGACA | |
| | GCGGCGGCAGCAGCGGCGGCAGCAGCGGCAGCGAGACACCC | |
| | GGCACCAGCGAGAGCGCCACCCCCGAGAGCAGCGGCGGCAG | |
| | CAGCGGCGGCAGCGACAAGAAGTACAGCATCGGCCTGGCCA | |
| | TCGGCACCAACAGCGTGGGCTGGGCCGTGATCACCGACGAGT | |
| | ACAAGGTGCCCAGCAAGAAGTTCAAGGTGCTGGGCAACACC | |
| | GACCGGCACAGCATCAAGAAGAACCTGATCGGCGCCCTGCTG | |
| | TTCGACAGCGGCGAGACAGCCGAGGCCACCCGGCTGAAGCG | |
| | GACCGCCCGGCGGCGGTACACCCGGCGGAAGAACCGGATCT | |
| | GCTACCTGCAGGAGATCTTCAGCAACGAGATGGCCAAGGTGG | |
| | ACGACAGCTTCTTCCACCGGCTGGAGGAGAGCTTCCTGGTGG | |
| | AGGAGGACAAGAAGCACGAGCGGCACCCCATCTTCGGCAAC | |
| | ATCGTGGACGAGGTGGCCTACCACGAGAAGTACCCCACCATC | |
| | TACCACCTGCGGAAGAAGCTGGTGGACAGCACCGACAAGGC | |
| | CGACCTGCGGCTGATCTACCTGGCCCTGGCCCACATGATCAA | |
| | GTTCCGGGGCCACTTCCTGATCGAGGGCGACCTGAACCCCGA | |
| | CAACAGCGACGTGGACAAGCTGTTCATCCAGCTGGTGCAGAC | |
| | CTACAACCAGCTGTTCGAGGAGAACCCCATCAACGCCAGCGG | |
| | CGTGGACGCCAAGGCCATCCTGAGCGCCCGGCTGAGCAAGAG | |
| | CCGGCGGCTGGAGAACCTGATCGCCCAGCTGCCCGGCGAGAA | |
| | GAAGAACGGCCTGTTCGGCAACCTGATCGCCCTGAGCCTGGG | |
| | CCTGACCCCCAACTTCAAGAGCAACTTCGACCTGGCCGAGGA | |
| | CGCCAAGCTGCAGCTGAGCAAGGACACCTACGACGACGACCT | |
| | GGACAACCTGCTGGCCCAGATCGGCGACCAGTACGCCGACCT | |
| | GTTCCTGGCCGCCAAGAACCTGAGCGACGCCATCCTGCTGAG | |
| | CGACATCCTGCGGGTGAACACCGAGATCACCAAGGCCCCCCT | |
| | GAGCGCCAGCATGATCAAGCGGTACGACGAGCACCACCAGG | |
| | ACCTGACCCTGCTGAAGGCCCTGGTGCGGCAGCAGCTGCCCG | |
| | AGAAGTACAAGGAGATCTTCTTCGACCAGAGCAAGAACGGCT | |
| | ACGCCGGCTACATCGACGGCGGCGCCAGCCAGGAGGAGTTCT | |
| | ACAAGTTCATCAAGCCCATCCTGGAGAAGATGGACGGCACCG | |
| | AGGAGCTGCTGGTGAAGCTGAACCGGGAGGACCTGCTGCGG | |
| | AAGCAGCGGACCTTCGACAACGGCAGCATCCCCCACCAGATC | |
| | CACCTGGGCGAGCTGCACGCCATCCTGCGGCGGCAGGAGGAC | |
| | TTCTACCCCTTCCTGAAGGACAACCGGGAGAAGATCGAGAAG | |
| | ATCCTGACCTTCCGGATCCCCTACTACGTGGGCCCCCTGGCCC | |
| | GGGGCAACAGCCGGTTCGCCTGGATGACCCGCAAGAGCGAG | |
| | GAGACAATCACCCCCTGGAACTTCGAGGAGGTGGTGGACAAG | |
| | GGCGCCAGCGCCCAGAGCTTCATCGAGCGGATGACCAACTTC | |
| | GACAAGAACCTGCCCAACGAGAAGGTGCTGCCCAAGCACAG | |
| | CCTGCTGTACGAGTACTTCACCGTGTACAACGAGCTGACCAA | |
| | GGTGAAGTACGTGACCGAGGGCATGCGGAAGCCCGCCTTCCT | |
| | GAGCGGCGAGCAGAAGAAGGCCATCGTGGACCTGCTGTTCAA | |
| | GACCAACCGGAAGGTGACCGTGAAGCAGCTGAAGGAGGACT | |
| | ACTTCAAGAAGATCGAGTGCTTCGACAGCGTGGAGATCAGCG | |
| | GCGTGGAGGACCGGTTCAACGCCAGCCTGGGCACCTACCACG | |
| | ACCTGCTGAAGATCATCAAGGACAAGGACTTCCTGGACAACG | |
| | AGGAGAACGAGGACATCCTGGAGGACATCGTGCTGACCCTGA | |
| | CCCTGTTCGAGGACCGGGAGATGATCGAGGAGCGGCTGAAG | |
| | ACCTACGCCCACCTGTTCGACGACAAGGTGATGAAGCAGCTG | |
| | AAGCGGCGGCGGTACACCGGCTGGGGCCGGCTGAGCCGGAA | |
| | GCTGATCAACGGCATCCGGGACAAGCAGAGCGGCAAGACCA | |
| | TCCTGGACTTCCTCAAGAGCGACGGCTTCGCCAACGCCAACT | |
| | TCATGCAGCTGATCCACGACGACAGCCTGACCTTCAAGGAGG | |
| | ACATCCAGAAGGCCCAGGTGAGCGGCCAGGGCGACAGCCTG | |
| | CACGAGCACATCGCCAACCTGGCCGGCAGCCCCGCCATCAAG | |
| | AAGGGCATCCTGCAGACCGTGAAGGTGGTGGACGAGCTGGTG | |
| | AAGGTGATGGGCCGGCACAAGCCCGAGAACATCGTGATCGA | |
| | GATGGCCCGGGAGAACCAGACCACCCAGAAGGGCCAGAAGA | |
| | ACAGCCGGGAGCGGATGAAGCGGATCGAGGAGGGCATCAAG | |
| | GAGCTGGGCAGCCAGATCCTGAAGGAGCACCCCGTGGAGAA | |
| | CACCCAGCTGCAGAACGAGAAGCTGTACCTGTACTACCTGCA | |
| | GAACGGCCGGGACATGTACGTGGACCAGGAGCTGGACATCA | |
| | ACCGGCTGAGCGACTACGACGTGGACCACATCGTGCCCCAGA | |
| | GCTTCCTGAAGGACGACAGCATCGACAACAAGGTGCTGACCC | |
| | GGAGCGACAAGAACCGGGGCAAGAGCGACAACGTGCCCAGC | |
| | GAGGAGGTGGTGAAGAAGATGAAGAACTACTGGCGGCAGCT | |
| | GCTGAACGCCAAGCTGATCACCCAGCGGAAGTTCGACAACCT | |
| | GACCAAGGCCGAGCGGGGCGGCCTGAGCGAGCTGGACAAGG | |
| | CCGGCTTCATCAAGCGGCAGCTGGTGGAGACACGGCAGATCA | |
| | CCAAGCACGTGGCCCAGATCCTGGACAGCCGGATGAACACCA | |
| | AGTACGACGAGAACGACAAGCTGATCCGGGAGGTGAAGGTG | |
| | ATCACCCTCAAGAGCAAGCTGGTGAGCGACTTCCGGAAGGAC | |
| | TTCCAGTTCTACAAGGTGCGGGAGATCAACAACTACCACCAC | |
| | GCCCACGACGCCTACCTGAACGCCGTGGTGGGCACCGCCCTG | |
| | ATCAAGAAGTACCCCAAGCTGGAGAGCGAGTTCGTGTACGGC | |
| | GACTACAAGGTGTACGACGTGCGGAAGATGATCGCCAAGAG | |
| | CGAGCAGGAGATCGGCAAGGCCACCGCCAAGTACTTCTTCTA | |
| | CAGCAACATCATGAACTTCTTCAAGACCGAGATCACCCTGGC | |
| | CAACGGCGAGATCCGGAAGCGGCCCCTGATCGAGACAAACG | |
| | GCGAGACAGGCGAGATCGTGTGGGACAAGGGCCGGGACTTC | |
| | GCCACCGTGCGGAAGGTGCTGAGCATGCCCCAGGTGAACATC | |
| | GTGAAGAAGACCGAGGTGCAGACCGGCGGCTTCAGCAAGGA | |
| | GAGCATCCTGCCCAAGCGGAACAGCGACAAGCTGATCGCCCG | |
| | GAAGAAGGACTGGGACCCCAAGAAGTACGGCGGCTTCGACA | |
| | GCCCCACCGTGGCCTACAGCGTGCTGGTGGTGGCCAAGGTGG | |
| | AGAAGGGCAAGAGCAAGAAGCTCAAGAGCGTGAAGGAGCTG | |
| | CTGGGCATCACCATCATGGAGCGGAGCAGCTTCGAGAAGAAC | |
| | CCCATCGACTTCCTGGAGGCCAAGGGCTACAAGGAGGTGAAG | |
| | AAGGACCTGATCATCAAGCTGCCCAAGTACAGCCTGTTCGAG | |
| | CTGGAGAACGGCCGGAAGCGGATGCTGGCCAGCGCCGGCGA | |
| | GCTGCAGAAGGGCAACGAGCTGGCCCTGCCCAGCAAGTACGT | |
| | GAACTTCCTGTACCTGGCCAGCCACTACGAGAAGCTGAAGGG | |
| | CAGCCCCGAGGACAACGAGCAGAAGCAGCTGTTCGTGGAGC | |
| | AGCACAAGCACTACCTGGACGAGATCATCGAGCAGATCAGCG | |
| | AGTTCAGCAAGCGGGTGATCCTGGCCGACGCCAACCTGGACA | |
| | AGGTGCTGAGCGCCTACAACAAGCACCGGGACAAGCCCATCC | |
| | GGGAGCAGGCCGAGAACATCATCCACCTGTTCACCCTGACCA | |
| | ACCTGGGCGCCCCCGCCGCCTTCAAGTACTTCGACACCACCA | |
| | TCGACCGGAAGCGGTACACCAGCACCAAGGAGGTGCTGGAC | |
| | GCCACCCTGATCCACCAGAGCATCACCGGCCTGTACGAGACA | |
| | CGGATCGACCTGAGCCAGCTGGGCGGCGACGAGGGCGCCGA | |
| | CAAGCGGACCGCCGACGGCAGCGAGTTCGAGAGCCCCAAGA | |
| | AGAAGCGGAAGGTGTGA | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| MA045 | MSEVEFSHEYWMRHALTLAKRARDEREVPVGAVLVLNNRVIGE | 722 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANA | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEKLYLYYLQNGRDM | |
| | YVDQELDINRLSDYDVDHIVPQSFLKDDSIDNKVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPKLESEFVYGDYKVYDVRKMIAKSEQEIGKATAKYFFYS | |
| | NIMNFFKTEITLANGEIRKRPLIETNGETGEIVWDKGRDFATVRK | |
| | VLSMPQVNIVKKTEVQTGGFSKESILPKRNSDKLIARKKDWDPK | |
| | KYGGFDSPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

MA064 (Hypa) mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | Au′GAGCGAGGu′GGAGu′u′CAGCCACGAGu′ACu′GGAu′GCGG | 723 |
| fidelity | CACGCCCu′GACCCu′GGCCAAGCGGGCCCGGGACGAGCGGGA | |
| ABE | GGu′GCCCGu′GGGCGCCGu′GCu′GGu′GCu′GAACAACCGGGu′G | |
| CDS | Au′CGGCGAGGGCu′GGAACCGGGCCAu′CGGCCu′GCACGACC | |
| | CCACCGCCCACGCCGAGAu′CAu′GGCCCu′GCGGCAGGGCGGC | |
| | Cu′GGu′GAu′GCAGAACu′ACCGGCu′GAu′CGACGCCACCCu′Gu′ | |
| | ACGu′GACCu′u′CGAGCCCu′GCGu′GAu′Gu′GCGCCGGCGCCAu′ | |
| | GAu′CCACAGCCGGAu′CGGCCGGGu′GGu′Gu′u′CGGCGu′GCGG | |
| | AACGCCAAGACCGGCGCCGCCGGCAGCCu′GAu′GGACGu′GCu | |
| | ′GCACCACCCCGGCAu′GAACCACCGGGu′GGAGAu′CACCGAG | |
| | GGCAu′CCu′GGCCGACGAGu′GCGCCGCCCu′GCu′Gu′GCCGGu′ | |
| | u′Cu′u′CCGGAu′GCCCCGGCGGGu′Gu′u′CAACGCCCAGAAGAA | |
| | GGCCCAGAGCAGCACCGACAGCGGCGGCAGCAGCGGCGGCA | |
| | GCAGCGGCAGCGAGACACCCGGCACCAGCGAGAGCGCCACC | |
| | CCCGAGAGCAGCGGCGGCAGCAGCGGCGGCAGCGACAAGAA | |
| | Gu′ACAGCAu′CGGCCu′GGCCAu′CGGCACCAACAGCGu′GGGC | |
| | u′GGGCCGu′GAu′CACCGACGAGu′ACAAGGu′GCCCAGCAAGA | |
| | AGu′u′CAAGGu′GCu′GGGCAACACCGACCGGCACAGCAu′CAA | |
| | GAAGAACCu′GAu′CGGCGCCCu′GCu′Gu′u′CGACAGCGGCGAG | |
| | ACAGCCGAGGCCACCCGGCu′GAAGCGGACCGCCCGGCGGCG | |
| | Gu′ACACCCGGCGGAAGAACCGGAu′Cu′GCu′ACCu′GCAGGAG | |
| | Au′Cu′u′CAGCAACGAGAu′GGCCAAGGu′GGACGACAGCu′u′Cu | |
| | ′u′CCACCGGCu′GGAGGAGAGCu′u′CCu′GGu′GGAGGAGGACA | |
| | AGAAGCACGAGCGGCACCCCAu′Cu′u′CGGCAACAu′CGu′GGA | |
| | CGAGGu′GGCCu′ACCACGAGAAGu′ACCCCACCAu′Cu′ACCAC | |
| | Cu′GCGGAAGAAGCu′GGu′GGACAGCACCGACAAGGCCGACCU | |
| | ′GCGGCu′GAu′Cu′ACCu′GGCCCu′GGCCCACAu′GAu′CAAGu′u′ | |
| | CCGGGGCCACu′u′CCu′GAu′CGAGGGCGACCu′GAACCCCGAC | |
| | AACAGCGACGu′GGACAAGCu′Gu′u′CAu′CCAGCu′GGu′GCAGA | |
| | CCu′ACAACCAGCu′Gu′u′CGAGGAGAACCCCAu′CAACGCCAG | |
| | CGGCGu′GGACGCCAAGGCCAu′CCu′GAGCGCCCGGCu′GAGC | |
| | AAGAGCCGGCGGCu′GGAGAACCu′GAu′CGCCCAGCu′GCCCG | |
| | GCGAGAAGAAGAACGGCCu′Gu′u′CGGCAACCu′GAu′CGCCCu′ | |
| | GAGCCu′GGGCCu′GACCCCCAACu′u′CAAGAGCAACu′u′CGAC | |
| | Cu′GGCCGAGGACGCCAAGCu′GCAGCu′GAGCAAGGACACCu′ | |
| | ACGACGACGACCu′GGACAACCu′GCu′GGCCCAGAu′CGGCGA | |
| | CCAGu′ACGCCGACCu′Gu′u′CCu′GGCCGCCAAGAACCu′GAGC | |
| | GACGCCAu′CCu′GCu′GAGCGACAu′CCu′GCGGGu′GAACACCG | |
| | AGAu′CACCAAGGCCCCCCu′GAGCGCCAGCAu′GAu′CAAGCG | |
| | Gu′ACGACGAGCACCACCAGGACCu′GACCCu′GCu′GAAGGCC | |
| | Cu′GGu′GCGGCAGCAGCu′GCCCGAGAAGu′ACAAGGAGAu′Cu′ | |
| | u′Cu′u′CGACCAGAGCAAGAACGGCu′ACGCCGGCu′ACAu′CGA | |
| | CGGCGGCGCCAGCCAGGAGGAGu′u′Cu′ACAAGu′u′CAu′CAAG | |
| | CCCAu′CCu′GGAGAAGAu′GGACGGCACCGAGGAGCu′GCu′GG | |
| | u′GAAGCu′GAACCGGGAGGACCu′GCu′GCGGAAGCAGCGGAC | |
| | Cu′u′CGACAACGGCAGCAu′CCCCCACCAGAu′CCACCu′GGGC | |
| | GAGCu′GCACGCCAu′CCu′GCGGCGGCAGGAGGACu′u′Cu′ACC | |
| | CCu′u′CCu′GAAGGACAACCGGGAGAAGAu′CGAGAAGAu′CCu′ | |
| | GACCu′u′CCGGAu′CCCCu′ACu′ACGu′GGGCCCCCu′GGCCCGG | |
| | GGCAACAGCCGGu′u′CGCCu′GGAu′GACCCGCAAGAGCGAGG | |
| | AGACAAu′CACCCCCu′GGAACu′u′CGAGGAGGu′GGu′GGACAA | |
| | GGGCGCCAGCGCCCAGAGCu′u′CAu′CGAGCGGAu′GACCAACu | |
| | ′u′CGACAAGAACCu′GCCCAACGAGAAGGu′GCu′GCCCAAGCA | |
| | CAGCCu′GCu′Gu′ACGAGu′ACu′u′CACCGu′Gu′ACAACGAGCu′ | |
| | GACCAAGGu′GAAGu′ACGu′GACCGAGGGCAu′GCGGAAGCCC | |
| | GCCu′u′CCu′GAGCGGCGAGCAGAAGAAGGCCAu′CGu′GGACC | |
| | u′GCu′Gu′u′CAAGACCAACCGGAAGGu′GACCGu′GAAGCAGCu′ | |
| | GAAGGAGGACu′ACu′u′CAAGAAGAu′CGAGu′GCu′u′CGACAG | |
| | CGu′GGAGAu′CAGCGGCGu′GGAGGACCGGu′u′CAACGCCAGC | |
| | Cu′GGGCACCu′ACCACGACCu′GCu′GAAGAu′CAu′CAAGGACA | |
| | AGGACu′u′CCu′GGACAACGAGGAGAACGAGGACAu′CCu′GGA | |
| | GGACAu′CGu′GCu′GACCCu′GACCCu′Gu′u′CGAGGACCGGGAG | |
| | Au′GAu′CGAGGAGCGGCu′GAAGACCu′ACGCCCACCu′Gu′u′CG | |
| | ACGACAAGGu′GAu′GAAGCAGCu′GAAGCGGCGGCGGu′ACAC | |
| | CGGCu′GGGGCCGGCu′GAGCCGGAAGCu′GAu′CAACGGCAu′C | |
| | CGGGACAAGCAGAGCGGCAAGACCAu′CCu′GGACu′u′CCu′CA | |
| | AGAGCGACGGCu′u′CGCCAACCGGGCCu′u′CGCCGCCCu′GAu | |
| | ′CGCCGACGACAGCCu′GACCu′u′CAAGGAGGACAu′CCAGAAG | |
| | GCCCAGGu′GAGCGGCCAGGGCGACAGCCu′GCACGAGCACAu | |
| | ′CGCCAACCu′GGCCGGCAGCCCCGCCAu′CAAGAAGGGCAu′C | |
| | Cu′GCAGACCGu′GAAGGu′GGu′GGACGAGCu′GGu′GAAGGu′G | |
| | Au′GGGCCGGCACAAGCCCGAGAACAu′CGu′GAu′CGAGAu′GG | |
| | CCCGGGAGAACCAGACCACCCAGAAGGGCCAGAAGAACAGC | |
| | CGGGAGCGGAu′GAAGCGGAu′CGAGGAGGGCAu′CAAGGAGC | |
| | u′GGGCAGCCAGAu′CCu′GAAGGAGCACCCCGu′GGAGAACAC | |
| | CCAGCu′GCAGAACGAGAAGCu′Gu′ACCu′Gu′ACu′ACCu′GCAG | |
| | AACGGCCGGGACAu′Gu′ACGu′GGACCAGGAGCu′GGACAu′CA | |
| | ACCGGCu′GAGCGACu′ACGACGu′GGACCACAu′CGu′GCCCCA | |
| | GAGCu′u′CCu′GAAGGACGACAGCAu′CGACAACAAGGu′GCu′G | |
| | ACCCGGAGCGACAAGAACCGGGGCAAGAGCGACAACGu′GCC | |
| | CAGCGAGGAGGu′GGu′GAAGAAGAu′GAAGAACu′ACu′GGCG | |
| | GCAGCu′GCu′GAACGCCAAGCu′GAu′CACCCAGCGGAAGu′u′C | |
| | GACAACCu′GACCAAGGCCGAGCGGGGCGGCCu′GAGCGAGCu | |
| | ′GGACAAGGCCGGCu′u′CAu′CAAGCGGCAGCu′GGu′GGAGAC | |
| | ACGGCAGAu′CACCAAGCACGu′GGCCCAGAu′CCu′GGACAGC | |
| | CGGAu′GAACACCAAGu′ACGACGAGAACGACAAGCu′GAu′CC | |
| | GGGAGGu′GAAGGu′GAu′CACCCu′CAAGAGCAAGCu′GGu′GA | |
| | GCGACu′u′CCGGAAGGACu′u′CCAGu′u′Cu′ACAAGGu′GCGGG | |
| | AGAu′CAACAACu′ACCACCACGCCCACGACGCCu′ACCu′GAAC | |
| | GCCGu′GGu′GGGCACCGCCCu′GAu′CAAGAAGu′ACCCCAAGC | |
| | u′GGAGAGCGAGu′u′CGu′Gu′ACGGCGACu′ACAAGGu′Gu′ACG | |
| | ACGu′GCGGAAGAu′GAu′CGCCAAGAGCGAGCAGGAGAu′CGG | |
| | CAAGGCCACCGCCAAGu′ACu′u′Cu′u′Cu′ACAGCAACAu′CAu′G | |
| | AACu′u′Cu′u′CAAGACCGAGAu′CACCCu′GGCCAACGGCGAGA | |
| | u′CCGGAAGCGGCCCCu′GAu′CGAGACAAACGGCGAGACAGG | |
| | CGAGAu′CGu′Gu′GGGACAAGGGCCGGGACu′u′CGCCACCGu′G | |
| | CGGAAGGu′GCu′GAGCAu′GCCCCAGGu′GAACAu′CGu′GAAGA | |
| | AGACCGAGGu′GCAGACCGGCGGCu′u′CAGCAAGGAGAGCAu′ | |
| | CCu′GCCCAAGCGGAACAGCGACAAGCu′GAu′CGCCCGGAAG | |
| | AAGGACu′GGGACCCCAAGAAGu′ACGGCGGCu′u′CGACAGCC | |
| | CCACCGu′GGCCu′ACAGCGu′GCu′GGu′GGu′GGCCAAGGu′GGA | |
| | GAAGGGCAAGAGCAAGAAGCu′CAAGAGCGu′GAAGGAGCu′G | |
| | Cu′GGGCAu′CACCAu′CAu′GGAGCGGAGCAGCu′u′CGAGAAGA | |
| | ACCCCAu′CGACu′u′CCu′GGAGGCCAAGGGCu′ACAAGGAGGu′ | |
| | GAAGAAGGACCu′GAu′CAu′CAAGCu′GCCCAAGu′ACAGCCu′G | |
| | u′u′CGAGCu′GGAGAACGGCCGGAAGCGGAu′GCu′GGCCAGCG | |
| | CCGGCGAGCu′GCAGAAGGGCAACGAGCu′GGCCCu′GCCCAGC | |
| | AAGu′ACGu′GAACu′u′CCu′Gu′ACCu′GGCCAGCCACu′ACGAGA | |
| | AGCu′GAAGGGCAGCCCCGAGGACAACGAGCAGAAGCAGCu′ | |
| | Gu′u′CGu′GGAGCAGCACAAGCACu′ACCu′GGACGAGAu′CAu′C | |
| | GAGCAGAu′CAGCGAGu′u′CAGCAAGCGGGu′GAu′CCu′GGCCG | |
| | ACGCCAACCu′GGACAAGGu′GCu′GAGCGCCu′ACAACAAGCA | |
| | CCGGGACAAGCCCAu′CCGGGAGCAGGCCGAGAACAu′CAu′CC | |
| | ACCu′Gu′u′CACCCu′GACCAACCu′GGGCGCCCCCGCCGCCu′u′ | |
| | CAAGu′ACu′u′CGACACCACCAu′CGACCGGAAGCGGu′ACACC | |
| | AGCACCAAGGAGGu′GCu′GGACGCCACCCu′GAu′CCACCAGA | |
| | GCAu′CACCGGCCu′Gu′ACGAGACACGGAu′CGACCu′GAGCCA | |
| | GCu′GGGCGGCGACGAGGGCGCCGACAAGCGGACCGCCGACG | |
| | GCAGCGAGu′u′CGAGAGCCCCAAGAAGAAGCGGAAGGu′Gu′G | |
| | A | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| MA064 | MSEVEFSHEYWMRHALTLAKRARDEREVPVGAVLVLNNRVIGE | 724 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANR | |
| | AFAALIADDSLTFKEDIQKAQVSGQGDSLHEHIANLAGSPAIKKG | |
| | ILQTVKVVDELVKVMGRHKPENIVIEMARENQTTQKGQKNSRE | |
| | RMKRIEEGIKELGSQILKEHPVENTQLQNEKLYLYYLQNGRDMY | |
| | VDQELDINRLSDYDVDHIVPQSFLKDDSIDNKVLTRSDKNRGKS | |
| | DNVPSEEVVKKMKNYWRQLLNAKLITQRKFDNLTKAERGGLSE | |
| | LDKAGFIKRQLVETRQITKHVAQILDSRMNTKYDENDKLIREVK | |
| | VITLKSKLVSDFRKDFQFYKVREINNYHHAHDAYLNAVVGTALI | |
| | KKYPKLESEFVYGDYKVYDVRKMIAKSEQEIGKATAKYFFYSNI | |
| | MNFFKTEITLANGEIRKRPLIETNGETGEIVWDKGRDFATVRKVL | |
| | SMPQVNIVKKTEVQTGGFSKESILPKRNSDKLIARKKDWDPKKY | |
| | GGFDSPTVAYSVLVVAKVEKGKSKKLKSVKELLGITIMERSSFE | |
| | KNPIDFLEAKGYKEVKKDLIIKLPKYSLFELENGRKRMLASAGEL | |
| | QKGNELALPSKYVNFLYLASHYEKLKGSPEDNEQKQLFVEQHK | |
| | HYLDEIIEQISEFSKRVILADANLDKVLSAYNKHRDKPIREQAENI | |
| | IHLFTLTNLGAPAAFKYFDTTIDRKRYTSTKEVLDATLIHQSITGL | |
| | YETRIDLSQLGGDEGADKRTADGSEFESPKKKRKV | |

MA065 (K855A) mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | Au′GAGCGAGGu′GGAGu′u′CAGCCACGAGu′ACu′GGAu′GCGG | 725 |
| fidelity | CACGCCCu′GACCCu′GGCCAAGCGGGCCCGGGACGAGCGGGA | |
| ABE | GGu′GCCCGu′GGGCGCCGu′GCu′GGu′GCu′GAACAACCGGGu′G | |
| CDS | Au′CGGCGAGGGCu′GGAACCGGGCCAu′CGGCCu′GCACGACC | |
| | CCACCGCCCACGCCGAGAu′CAu′GGCCCu′GCGGCAGGGCGGC | |
| | Cu′GGu′GAu′GCAGAACu′ACCGGCu′GAu′CGACGCCACCCu′Gu′ | |
| | ACGu′GACCu′u′CGAGCCCu′GCGu′GAu′Gu′GCGCCGGCGCCAu′ | |
| | GAu′CCACAGCCGGAu′CGGCCGGGu′GGu′Gu′u′CGGCGu′GCGG | |
| | AACGCCAAGACCGGCGCCGCCGGCAGCCu′GAu′GGACGu′GCu | |
| | ′GCACCACCCCGGCAu′GAACCACCGGGu′GGAGAu′CACCGAG | |
| | GGCAu′CCu′GGCCGACGAGu′GCGCCGCCCu′GCu′Gu′GCCGGu′ | |
| | u′Cu′u′CCGGAu′GCCCCGGCGGGu′Gu′u′CAACGCCCAGAAGAA | |
| | GGCCCAGAGCAGCACCGACAGCGGCGGCAGCAGCGGCGGCA | |
| | GCAGCGGCAGCGAGACACCCGGCACCAGCGAGAGCGCCACC | |
| | CCCGAGAGCAGCGGCGGCAGCAGCGGCGGCAGCGACAAGAA | |
| | Gu′ACAGCAu′CGGCCu′GGCCAu′CGGCACCAACAGCGu′GGGC | |
| | u′GGGCCGu′GAu′CACCGACGAGu′ACAAGGu′GCCCAGCAAGA | |
| | AGu′u′CAAGGu′GCu′GGGCAACACCGACCGGCACAGCAu′CAA | |
| | GAAGAACCu′GAu′CGGCGCCCu′GCu′Gu′u′CGACAGCGGCGAG | |
| | ACAGCCGAGGCCACCCGGCu′GAAGCGGACCGCCCGGCGGCG | |
| | Gu′ACACCCGGCGGAAGAACCGGAu′Cu′GCu′ACCu′GCAGGAG | |
| | Au′Cu′u′CAGCAACGAGAu′GGCCAAGGu′GGACGACAGCu′u′Cu | |
| | ′u′CCACCGGCu′GGAGGAGAGCu′u′CCu′GGu′GGAGGAGGACA | |
| | AGAAGCACGAGCGGCACCCCAu′Cu′u′CGGCAACAu′CGu′GGA | |
| | CGAGGu′GGCCu′ACCACGAGAAGu′ACCCCACCAu′Cu′ACCAC | |
| | Cu′GCGGAAGAAGCu′GGu′GGACAGCACCGACAAGGCCGACCU | |
| | ′GCGGCu′GAu′Cu′ACCu′GGCCCu′GGCCCACAu′GAu′CAAGu′u′ | |
| | CCGGGGCCACu′u′CCu′GAu′CGAGGGCGACCu′GAACCCCGAC | |
| | AACAGCGACGu′GGACAAGCu′Gu′u′CAu′CCAGCu′GGu′GCAGA | |
| | CCu′ACAACCAGCu′Gu′u′CGAGGAGAACCCCAu′CAACGCCAG | |
| | CGGCGu′GGACGCCAAGGCCAu′CCu′GAGCGCCCGGCu′GAGC | |
| | AAGAGCCGGCGGCu′GGAGAACCu′GAu′CGCCCAGCu′GCCCG | |
| | GCGAGAAGAAGAACGGCCu′Gu′u′CGGCAACCu′GAu′CGCCCu′ | |
| | GAGCCu′GGGCCu′GACCCCCAACu′u′CAAGAGCAACu′u′CGAC | |
| | Cu′GGCCGAGGACGCCAAGCu′GCAGCu′GAGCAAGGACACCu′ | |
| | ACGACGACGACCu′GGACAACCu′GCu′GGCCCAGAu′CGGCGA | |
| | CCAGu′ACGCCGACCu′Gu′u′CCu′GGCCGCCAAGAACCu′GAGC | |
| | GACGCCAu′CCu′GCu′GAGCGACAu′CCu′GCGGGu′GAACACCG | |
| | AGAu′CACCAAGGCCCCCCu′GAGCGCCAGCAu′GAu′CAAGCG | |
| | Gu′ACGACGAGCACCACCAGGACCu′GACCCu′GCu′GAAGGCC | |
| | Cu′GGu′GCGGCAGCAGCu′GCCCGAGAAGu′ACAAGGAGAu′Cu′ | |
| | u′Cu′u′CGACCAGAGCAAGAACGGCu′ACGCCGGCu′ACAu′CGA | |
| | CGGCGGCGCCAGCCAGGAGGAGu′u′Cu′ACAAGu′u′CAu′CAAG | |
| | CCCAu′CCu′GGAGAAGAu′GGACGGCACCGAGGAGCu′GCu′GG | |
| | u′GAAGCu′GAACCGGGAGGACCu′GCu′GCGGAAGCAGCGGAC | |
| | Cu′u′CGACAACGGCAGCAu′CCCCCACCAGAu′CCACCu′GGGC | |
| | GAGCu′GCACGCCAu′CCu′GCGGCGGCAGGAGGACu′u′Cu′ACC | |
| | CCu′u′CCu′GAAGGACAACCGGGAGAAGAu′CGAGAAGAu′CCu′ | |
| | GACCu′u′CCGGAu′CCCCu′ACu′ACGu′GGGCCCCCu′GGCCCGG | |
| | GGCAACAGCCGGu′u′CGCCu′GGAu′GACCCGCAAGAGCGAGG | |
| | AGACAAu′CACCCCCu′GGAACu′u′CGAGGAGGu′GGu′GGACAA | |
| | GGGCGCCAGCGCCCAGAGCu′u′CAu′CGAGCGGAu′GACCAACu | |
| | ′u′CGACAAGAACCu′GCCCAACGAGAAGGu′GCu′GCCCAAGCA | |
| | CAGCCu′GCu′Gu′ACGAGu′ACu′u′CACCGu′Gu′ACAACGAGCu′ | |
| | GACCAAGGu′GAAGu′ACGu′GACCGAGGGCAu′GCGGAAGCCC | |
| | GCCu′u′CCu′GAGCGGCGAGCAGAAGAAGGCCAu′CGu′GGACC | |
| | u′GCu′Gu′u′CAAGACCAACCGGAAGGu′GACCGu′GAAGCAGCu′ | |
| | GAAGGAGGACu′ACu′u′CAAGAAGAu′CGAGu′GCu′u′CGACAG | |
| | CGu′GGAGAu′CAGCGGCGu′GGAGGACCGGu′u′CAACGCCAGC | |
| | Cu′GGGCACCu′ACCACGACCu′GCu′GAAGAu′CAu′CAAGGACA | |
| | AGGACu′u′CCu′GGACAACGAGGAGAACGAGGACAu′CCu′GGA | |
| | GGACAu′CGu′GCu′GACCCu′GACCCu′Gu′u′CGAGGACCGGGAG | |
| | Au′GAu′CGAGGAGCGGCu′GAAGACCu′ACGCCCACCu′Gu′u′CG | |
| | ACGACAAGGu′GAu′GAAGCAGCu′GAAGCGGCGGCGGu′ACAC | |
| | CGGCu′GGGGCCGGCu′GAGCCGGAAGCu′GAu′CAACGGCAu′C | |
| | CGGGACAAGCAGAGCGGCAAGACCAu′CCu′GGACu′u′CCu′CA | |
| | AGAGCGACGGCu′u′CGCCAACCGGAACu′u′CAu′GCAGCu′GAu′ | |
| | CCACGACGACAGCCu′GACCu′u′CAAGGAGGACAu′CCAGAAG | |
| | GCCCAGGu′GAGCGGCCAGGGCGACAGCCu′GCACGAGCACAu | |
| | ′CGCCAACCu′GGCCGGCAGCCCCGCCAu′CAAGAAGGGCAu′C | |
| | Cu′GCAGACCGu′GAAGGu′GGu′GGACGAGCu′GGu′GAAGGu′G | |
| | Au′GGGCCGGCACAAGCCCGAGAACAu′CGu′GAu′CGAGAu′GG | |
| | CCCGGGAGAACCAGACCACCCAGAAGGGCCAGAAGAACAGC | |
| | CGGGAGCGGAu′GAAGCGGAu′CGAGGAGGGCAu′CAAGGAGC | |
| | u′GGGCAGCCAGAu′CCu′GAAGGAGCACCCCGu′GGAGAACAC | |
| | CCAGCu′GCAGAACGAGAAGCu′Gu′ACCu′Gu′ACu′ACCu′GCAG | |
| | AACGGCCGGGACAu′Gu′ACGu′GGACCAGGAGCu′GGACAu′CA | |
| | ACCGGCu′GAGCGACu′ACGACGu′GGACCACAu′CGu′GCCCCA | |
| | GAGCu′u′CCu′GAAGGACGACAGCAu′CGACAACGCCGu′GCu′G | |
| | ACCCGGAGCGACAAGAACCGGGGCAAGAGCGACAACGu′GCC | |
| | CAGCGAGGAGGu′GGu′GAAGAAGAu′GAAGAACu′ACu′GGCG | |
| | GCAGCu′GCu′GAACGCCAAGCu′GAu′CACCCAGCGGAAGu′u′C | |
| | GACAACCu′GACCAAGGCCGAGCGGGGCGGCCu′GAGCGAGCu | |
| | ′GGACAAGGCCGGCu′u′CAu′CAAGCGGCAGCu′GGu′GGAGAC | |
| | ACGGCAGAu′CACCAAGCACGu′GGCCCAGAu′CCu′GGACAGC | |
| | CGGAu′GAACACCAAGu′ACGACGAGAACGACAAGCu′GAu′CC | |
| | GGGAGGu′GAAGGu′GAu′CACCCu′CAAGAGCAAGCu′GGu′GA | |
| | GCGACu′u′CCGGAAGGACu′u′CCAGu′u′Cu′ACAAGGu′GCGGG | |
| | AGAu′CAACAACu′ACCACCACGCCCACGACGCCu′ACCu′GAAC | |
| | GCCGu′GGu′GGGCACCGCCCu′GAu′CAAGAAGu′ACCCCAAGC | |
| | u′GGAGAGCGAGu′u′CGu′Gu′ACGGCGACu′ACAAGGu′Gu′ACG | |
| | ACGu′GCGGAAGAu′GAu′CGCCAAGAGCGAGCAGGAGAu′CGG | |
| | CAAGGCCACCGCCAAGu′ACu′u′Cu′u′Cu′ACAGCAACAu′CAu′G | |
| | AACu′u′Cu′u′CAAGACCGAGAu′CACCCu′GGCCAACGGCGAGA | |
| | u′CCGGAAGCGGCCCCu′GAu′CGAGACAAACGGCGAGACAGG | |
| | CGAGAu′CGu′Gu′GGGACAAGGGCCGGGACu′u′CGCCACCGu′G | |
| | CGGAAGGu′GCu′GAGCAu′GCCCCAGGu′GAACAu′CGu′GAAGA | |
| | AGACCGAGGu′GCAGACCGGCGGCu′u′CAGCAAGGAGAGCAu′ | |
| | CCu′GCCCAAGCGGAACAGCGACAAGCu′GAu′CGCCCGGAAG | |
| | AAGGACu′GGGACCCCAAGAAGu′ACGGCGGCu′u′CGACAGCC | |
| | CCACCGu′GGCCu′ACAGCGu′GCu′GGu′GGu′GGCCAAGGu′GGA | |
| | GAAGGGCAAGAGCAAGAAGCu′CAAGAGCGu′GAAGGAGCu′G | |
| | Cu′GGGCAu′CACCAu′CAu′GGAGCGGAGCAGCu′u′CGAGAAGA | |
| | ACCCCAu′CGACu′u′CCu′GGAGGCCAAGGGCu′ACAAGGAGGu′ | |
| | GAAGAAGGACCu′GAu′CAu′CAAGCu′GCCCAAGu′ACAGCCu′G | |
| | u′u′CGAGCu′GGAGAACGGCCGGAAGCGGAu′GCu′GGCCAGCG | |
| | CCGGCGAGCu′GCAGAAGGGCAACGAGCu′GGCCCu′GCCCAGC | |
| | AAGu′ACGu′GAACu′u′CCu′Gu′ACCu′GGCCAGCCACu′ACGAGA | |
| | AGCu′GAAGGGCAGCCCCGAGGACAACGAGCAGAAGCAGCu′ | |
| | Gu′u′CGu′GGAGCAGCACAAGCACu′ACCu′GGACGAGAu′CAu′C | |
| | GAGCAGAu′CAGCGAGu′u′CAGCAAGCGGGu′GAu′CCu′GGCCG | |
| | ACGCCAACCu′GGACAAGGu′GCu′GAGCGCCu′ACAACAAGCA | |
| | CCGGGACAAGCCCAu′CCGGGAGCAGGCCGAGAACAu′CAu′CC | |
| | ACCu′Gu′u′CACCCu′GACCAACCu′GGGCGCCCCCGCCGCCu′u′ | |
| | CAAGu′ACu′u′CGACACCACCAu′CGACCGGAAGCGGu′ACACC | |
| | AGCACCAAGGAGGu′GCu′GGACGCCACCCu′GAu′CCACCAGA | |
| | GCAu′CACCGGCCu′Gu′ACGAGACACGGAu′CGACCu′GAGCCA | |
| | GCu′GGGCGGCGACGAGGGCGCCGACAAGCGGACCGCCGACG | |
| | GCAGCGAGu′u′CGAGAGCCCCAAGAAGAAGCGGAAGGu′Gu′G | |
| | A | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| MA065 | MSEVEFSHEYWMRHALTLAKRARDEREVPVGAVLVLNNRVIGE | 726 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANR | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEKLYLYYLQNGRDM | |
| | YVDQELDINRLSDYDVDHIVPQSFLKDDSIDNAVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPKLESEFVYGDYKVYDVRKMIAKSEQEIGKATAKYFFYS | |
| | NIMNFFKTEITLANGEIRKRPLIETNGETGEIVWDKGRDFATVRK | |
| | VLSMPQVNIVKKTEVQTGGFSKESILPKRNSDKLIARKKDWDPK | |
| | KYGGFDSPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

MA066 (eSpCas9(1.1)) mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | Au′GAGCGAGGu′GGAGu′u′CAGCCACGAGu′ACu′GGAu′GCGG | 727 |
| fidelity | CACGCCCu′GACCCu′GGCCAAGCGGGCCCGGGACGAGCGGGA | |
| ABE | GGu′GCCCGu′GGGCGCCGu′GCu′GGu′GCu′GAACAACCGGGu′G | |
| CDS | Au′CGGCGAGGGCu′GGAACCGGGCCAu′CGGCCu′GCACGACC | |
| | CCACCGCCCACGCCGAGAu′CAu′GGCCCu′GCGGCAGGGCGGC | |
| | Cu′GGu′GAu′GCAGAACu′ACCGGCu′GAu′CGACGCCACCCu′Gu′ | |
| | ACGu′GACCu′u′CGAGCCCu′GCGu′GAu′Gu′GCGCCGGCGCCAu′ | |
| | GAu′CCACAGCCGGAu′CGGCCGGGu′GGu′Gu′u′CGGCGu′GCGG | |
| | AACGCCAAGACCGGCGCCGCCGGCAGCCu′GAu′GGACGu′GCu | |
| | ′GCACCACCCCGGCAu′GAACCACCGGGu′GGAGAu′CACCGAG | |
| | GGCAu′CCu′GGCCGACGAGu′GCGCCGCCCu′GCu′Gu′GCCGGu′ | |
| | u′Cu′u′CCGGAu′GCCCCGGCGGGu′Gu′u′CAACGCCCAGAAGAA | |
| | GGCCCAGAGCAGCACCGACAGCGGCGGCAGCAGCGGCGGCA | |
| | GCAGCGGCAGCGAGACACCCGGCACCAGCGAGAGCGCCACC | |
| | CCCGAGAGCAGCGGCGGCAGCAGCGGCGGCAGCGACAAGAA | |
| | Gu′ACAGCAu′CGGCCu′GGCCAu′CGGCACCAACAGCGu′GGGC | |
| | u′GGGCCGu′GAu′CACCGACGAGu′ACAAGGu′GCCCAGCAAGA | |
| | AGu′u′CAAGGu′GCu′GGGCAACACCGACCGGCACAGCAu′CAA | |
| | GAAGAACCu′GAu′CGGCGCCCu′GCu′Gu′u′CGACAGCGGCGAG | |
| | ACAGCCGAGGCCACCCGGCu′GAAGCGGACCGCCCGGCGGCG | |
| | Gu′ACACCCGGCGGAAGAACCGGAu′Cu′GCu′ACCu′GCAGGAG | |
| | Au′Cu′u′CAGCAACGAGAu′GGCCAAGGu′GGACGACAGCu′u′Cu | |
| | ′u′CCACCGGCu′GGAGGAGAGCu′u′CCu′GGu′GGAGGAGGACA | |
| | AGAAGCACGAGCGGCACCCCAu′Cu′u′CGGCAACAu′CGu′GGA | |
| | CGAGGu′GGCCu′ACCACGAGAAGu′ACCCCACCAu′Cu′ACCAC | |
| | Cu′GCGGAAGAAGCu′GGu′GGACAGCACCGACAAGGCCGACCU | |
| | ′GCGGCu′GAu′Cu′ACCu′GGCCCu′GGCCCACAu′GAu′CAAGu′u′ | |
| | CCGGGGCCACu′u′CCu′GAu′CGAGGGCGACCu′GAACCCCGAC | |
| | AACAGCGACGu′GGACAAGCu′Gu′u′CAu′CCAGCu′GGu′GCAGA | |
| | CCu′ACAACCAGCu′Gu′u′CGAGGAGAACCCCAu′CAACGCCAG | |
| | CGGCGu′GGACGCCAAGGCCAu′CCu′GAGCGCCCGGCu′GAGC | |
| | AAGAGCCGGCGGCu′GGAGAACCu′GAu′CGCCCAGCu′GCCCG | |
| | GCGAGAAGAAGAACGGCCu′Gu′u′CGGCAACCu′GAu′CGCCCu′ | |
| | GAGCCu′GGGCCu′GACCCCCAACu′u′CAAGAGCAACu′u′CGAC | |
| | Cu′GGCCGAGGACGCCAAGCu′GCAGCu′GAGCAAGGACACCu′ | |
| | ACGACGACGACCu′GGACAACCu′GCu′GGCCCAGAu′CGGCGA | |
| | CCAGu′ACGCCGACCu′Gu′u′CCu′GGCCGCCAAGAACCu′GAGC | |
| | GACGCCAu′CCu′GCu′GAGCGACAu′CCu′GCGGGu′GAACACCG | |
| | AGAu′CACCAAGGCCCCCCu′GAGCGCCAGCAu′GAu′CAAGCG | |
| | Gu′ACGACGAGCACCACCAGGACCu′GACCCu′GCu′GAAGGCC | |
| | Cu′GGu′GCGGCAGCAGCu′GCCCGAGAAGu′ACAAGGAGAu′Cu′ | |
| | u′Cu′u′CGACCAGAGCAAGAACGGCu′ACGCCGGCu′ACAu′CGA | |
| | CGGCGGCGCCAGCCAGGAGGAGu′u′Cu′ACAAGu′u′CAu′CAAG | |
| | CCCAu′CCu′GGAGAAGAu′GGACGGCACCGAGGAGCu′GCu′GG | |
| | u′GAAGCu′GAACCGGGAGGACCu′GCu′GCGGAAGCAGCGGAC | |
| | Cu′u′CGACAACGGCAGCAu′CCCCCACCAGAu′CCACCu′GGGC | |
| | GAGCu′GCACGCCAu′CCu′GCGGCGGCAGGAGGACu′u′Cu′ACC | |
| | CCu′u′CCu′GAAGGACAACCGGGAGAAGAu′CGAGAAGAu′CCu′ | |
| | GACCu′u′CCGGAu′CCCCu′ACu′ACGu′GGGCCCCCu′GGCCCGG | |
| | GGCAACAGCCGGu′u′CGCCu′GGAu′GACCCGCAAGAGCGAGG | |
| | AGACAAu′CACCCCCu′GGAACu′u′CGAGGAGGu′GGu′GGACAA | |
| | GGGCGCCAGCGCCCAGAGCu′u′CAu′CGAGCGGAu′GACCAACu | |
| | ′u′CGACAAGAACCu′GCCCAACGAGAAGGu′GCu′GCCCAAGCA | |
| | CAGCCu′GCu′Gu′ACGAGu′ACu′u′CACCGu′Gu′ACAACGAGCu′ | |
| | GACCAAGGu′GAAGu′ACGu′GACCGAGGGCAu′GCGGAAGCCC | |
| | GCCu′u′CCu′GAGCGGCGAGCAGAAGAAGGCCAu′CGu′GGACC | |
| | u′GCu′Gu′u′CAAGACCAACCGGAAGGu′GACCGu′GAAGCAGCu′ | |
| | GAAGGAGGACu′ACu′u′CAAGAAGAu′CGAGu′GCu′u′CGACAG | |
| | CGu′GGAGAu′CAGCGGCGu′GGAGGACCGGu′u′CAACGCCAGC | |
| | Cu′GGGCACCu′ACCACGACCu′GCu′GAAGAu′CAu′CAAGGACA | |
| | AGGACu′u′CCu′GGACAACGAGGAGAACGAGGACAu′CCu′GGA | |
| | GGACAu′CGu′GCu′GACCCu′GACCCu′Gu′u′CGAGGACCGGGAG | |
| | Au′GAu′CGAGGAGCGGCu′GAAGACCu′ACGCCCACCu′Gu′u′CG | |
| | ACGACAAGGu′GAu′GAAGCAGCu′GAAGCGGCGGCGGu′ACAC | |
| | CGGCu′GGGGCCGGCu′GAGCCGGAAGCu′GAu′CAACGGCAu′C | |
| | CGGGACAAGCAGAGCGGCAAGACCAu′CCu′GGACu′u′CCu′CA | |
| | AGAGCGACGGCu′u′CGCCAACCGGAACu′u′CAu′GCAGCu′GAu′ | |
| | CCACGACGACAGCCu′GACCu′u′CAAGGAGGACAu′CCAGAAG | |
| | GCCCAGGu′GAGCGGCCAGGGCGACAGCCu′GCACGAGCACAu | |
| | ′CGCCAACCu′GGCCGGCAGCCCCGCCAu′CAAGAAGGGCAu′C | |
| | Cu′GCAGACCGu′GAAGGu′GGu′GGACGAGCu′GGu′GAAGGu′G | |
| | Au′GGGCCGGCACAAGCCCGAGAACAu′CGu′GAu′CGAGAu′GG | |
| | CCCGGGAGAACCAGACCACCCAGAAGGGCCAGAAGAACAGC | |
| | CGGGAGCGGAu′GAAGCGGAu′CGAGGAGGGCAu′CAAGGAGC | |
| | u′GGGCAGCCAGAu′CCu′GAAGGAGCACCCCGu′GGAGAACAC | |
| | CCAGCu′GCAGAACGAGAAGCu′Gu′ACCu′Gu′ACu′ACCu′GCAG | |
| | AACGGCCGGGACAu′Gu′ACGu′GGACCAGGAGCu′GGACAu′CA | |
| | ACCGGCu′GAGCGACu′ACGACGu′GGACCACAu′CGu′GCCCCA | |
| | GAGCu′u′CCu′GGCCGACGACAGCAu′CGACAACAAGGu′GCu′G | |
| | ACCCGGAGCGACAAGAACCGGGGCAAGAGCGACAACGu′GCC | |
| | CAGCGAGGAGGu′GGu′GAAGAAGAu′GAAGAACu′ACu′GGCG | |
| | GCAGCu′GCu′GAACGCCAAGCu′GAu′CACCCAGCGGAAGu′u′C | |
| | GACAACCu′GACCAAGGCCGAGCGGGGCGGCCu′GAGCGAGCu | |
| | ′GGACAAGGCCGGCu′u′CAu′CAAGCGGCAGCu′GGu′GGAGAC | |
| | ACGGCAGAu′CACCAAGCACGu′GGCCCAGAu′CCu′GGACAGC | |
| | CGGAu′GAACACCAAGu′ACGACGAGAACGACAAGCu′GAu′CC | |
| | GGGAGGu′GAAGGu′GAu′CACCCu′CAAGAGCAAGCu′GGu′GA | |
| | GCGACu′u′CCGGAAGGACu′u′CCAGu′u′Cu′ACAAGGu′GCGGG | |
| | AGAu′CAACAACu′ACCACCACGCCCACGACGCCu′ACCu′GAAC | |
| | GCCGu′GGu′GGGCACCGCCCu′GAu′CAAGAAGu′ACCCCGCCC | |
| | u′GGAGAGCGAGu′u′CGu′Gu′ACGGCGACu′ACAAGGu′Gu′ACG | |
| | ACGu′GCGGAAGAu′GAu′CGCCAAGAGCGAGCAGGAGAu′CGG | |
| | CAAGGCCACCGCCAAGu′ACu′u′Cu′u′Cu′ACAGCAACAu′CAu′G | |
| | AACu′u′Cu′u′CAAGACCGAGAu′CACCCu′GGCCAACGGCGAGA | |
| | u′CCGGAAGGCCCCCCu′GAu′CGAGACAAACGGCGAGACAGG | |
| | CGAGAu′CGu′Gu′GGGACAAGGGCCGGGACu′u′CGCCACCGu′G | |
| | CGGAAGGu′GCu′GAGCAu′GCCCCAGGu′GAACAu′CGu′GAAGA | |
| | AGACCGAGGu′GCAGACCGGCGGCu′u′CAGCAAGGAGAGCAu′ | |
| | CCu′GCCCAAGCGGAACAGCGACAAGCu′GAu′CGCCCGGAAG | |
| | AAGGACu′GGGACCCCAAGAAGu′ACGGCGGCu′u′CGACAGCC | |
| | CCACCGu′GGCCu′ACAGCGu′GCu′GGu′GGu′GGCCAAGGu′GGA | |
| | GAAGGGCAAGAGCAAGAAGCu′CAAGAGCGu′GAAGGAGCu′G | |
| | Cu′GGGCAu′CACCAu′CAu′GGAGCGGAGCAGCu′u′CGAGAAGA | |
| | ACCCCAu′CGACu′u′CCu′GGAGGCCAAGGGCu′ACAAGGAGGu′ | |
| | GAAGAAGGACCu′GAu′CAu′CAAGCu′GCCCAAGu′ACAGCCu′G | |
| | u′u′CGAGCu′GGAGAACGGCCGGAAGCGGAu′GCu′GGCCAGCG | |
| | CCGGCGAGCu′GCAGAAGGGCAACGAGCu′GGCCCu′GCCCAGC | |
| | AAGu′ACGu′GAACu′u′CCu′Gu′ACCu′GGCCAGCCACu′ACGAGA | |
| | AGCu′GAAGGGCAGCCCCGAGGACAACGAGCAGAAGCAGCu′ | |
| | Gu′u′CGu′GGAGCAGCACAAGCACu′ACCu′GGACGAGAu′CAu′C | |
| | GAGCAGAu′CAGCGAGu′u′CAGCAAGCGGGu′GAu′CCu′GGCCG | |
| | ACGCCAACCu′GGACAAGGu′GCu′GAGCGCCu′ACAACAAGCA | |
| | CCGGGACAAGCCCAu′CCGGGAGCAGGCCGAGAACAu′CAu′CC | |
| | ACCu′Gu′u′CACCCu′GACCAACCu′GGGCGCCCCCGCCGCCu′u′ | |
| | CAAGu′ACu′u′CGACACCACCAu′CGACCGGAAGCGGu′ACACC | |
| | AGCACCAAGGAGGu′GCu′GGACGCCACCCu′GAu′CCACCAGA | |
| | GCAu′CACCGGCCu′Gu′ACGAGACACGGAu′CGACCu′GAGCCA | |
| | GCu′GGGCGGCGACGAGGGCGCCGACAAGCGGACCGCCGACG | |
| | GCAGCGAGu′u′CGAGAGCCCCAAGAAGAAGCGGAAGGu′Gu′G | |
| | A | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′ AGGAAGu′Cu′AGA | |
| MA066 | MSEVEFSHEYWMRHALTLAKRARDEREVPVGAVLVLNNRVIGE | 728 |
| amino | GWNRAIGLHDPTAHAEIMALRQGGLVMQNYRLIDATLYVTFEP | |
| acid | CVMCAGAMIHSRIGRVVFGVRNAKTGAAGSLMDVLHHPGMNH | |
| sequence | RVEITEGILADECAALLCRFFRMPRRVFNAQKKAQSSTDSGGSSG | |
| | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANR | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEKLYLYYLQNGRDM | |
| | YVDQELDINRLSDYDVDHIVPQSFLADDSIDNKVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPALESEFVYGDYKVYDVRKMIAKSEQEIGKATAKYFFYS | |
| | NIMNFFKTEITLANGEIRKAPLIETNGETGEIVWDKGRDFATVRK | |
| | VLSMPQVNIVKKTEVQTGGFSKESILPKRNSDKLIARKKDWDPK | |
| | KYGGFDSPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

MA067 (eSpCas9(1.0)) mRNA sequence:

| Region | Sequence | |
|---|---|---|
| 5′ | AGGAAAu′AAGAGAGAAAAGAAGAGu′AAGAAGAAAu′Au′AA | 689 |
| UTR | GAGCCACC | |
| High | Au′GAGCGAGGu′GGAGu′u′CAGCCACGAGu′ACu′GGAu′GCGG | 729 |
| fidelity | CACGCCCu′GACCCu′GGCCAAGCGGGCCCGGGACGAGCGGGA | |
| ABE | GGu′GCCCGu′GGGCGCCGu′GCu′GGu′GCu′GAACAACCGGGu′G | |
| CDS | Au′CGGCGAGGGCu′GGAACCGGGCCAu′CGGCCu′GCACGACC | |
| | CCACCGCCCACGCCGAGAu′CAu′GGCCCu′GCGGCAGGGCGGC | |
| | Cu′GGu′GAu′GCAGAACu′ACCGGCu′GAu′CGACGCCACCCu′Gu′ | |
| | ACGu′GACCu′u′CGAGCCCu′GCGu′GAu′Gu′GCGCCGGCGCCAu′ | |
| | GAu′CCACAGCCGGAu′CGGCCGGGu′GGu′Gu′u′CGGCGu′GCGG | |
| | AACGCCAAGACCGGCGCCGCCGGCAGCCu′GAu′GGACGu′GCu | |
| | ′GCACCACCCCGGCAu′GAACCACCGGGu′GGAGAu′CACCGAG | |
| | GGCAu′CCu′GGCCGACGAGu′GCGCCGCCCu′GCu′Gu′GCCGGu′ | |
| | u′Cu′u′CCGGAu′GCCCCGGCGGGu′Gu′u′CAACGCCCAGAAGAA | |
| | GGCCCAGAGCAGCACCGACAGCGGCGGCAGCAGCGGCGGCA | |
| | GCAGCGGCAGCGAGACACCCGGCACCAGCGAGAGCGCCACC | |
| | CCCGAGAGCAGCGGCGGCAGCAGCGGCGGCAGCGACAAGAA | |
| | Gu′ACAGCAu′CGGCCu′GGCCAu′CGGCACCAACAGCGu′GGGC | |
| | u′GGGCCGu′GAu′CACCGACGAGu′ACAAGGu′GCCCAGCAAGA | |
| | AGu′u′CAAGGu′GCu′GGGCAACACCGACCGGCACAGCAu′CAA | |
| | GAAGAACCu′GAu′CGGCGCCCu′GCu′Gu′u′CGACAGCGGCGAG | |
| | ACAGCCGAGGCCACCCGGCu′GAAGCGGACCGCCCGGCGGCG | |
| | Gu′ACACCCGGCGGAAGAACCGGAu′Cu′GCu′ACCu′GCAGGAG | |
| | Au′Cu′u′CAGCAACGAGAu′GGCCAAGGu′GGACGACAGCu′u′Cu | |
| | ′u′CCACCGGCu′GGAGGAGAGCu′u′CCu′GGu′GGAGGAGGACA | |
| | AGAAGCACGAGCGGCACCCCAu′Cu′u′CGGCAACAu′CGu′GGA | |
| | CGAGGu′GGCCu′ACCACGAGAAGu′ACCCCACCAu′Cu′ACCAC | |
| | Cu′GCGGAAGAAGCu′GGu′GGACAGCACCGACAAGGCCGACCU | |
| | ′GCGGCu′GAu′Cu′ACCu′GGCCCu′GGCCCACAu′GAu′CAAGu′u′ | |
| | CCGGGGCCACu′u′CCu′GAu′CGAGGGCGACCu′GAACCCCGAC | |
| | AACAGCGACGu′GGACAAGCu′Gu′u′CAu′CCAGCu′GGu′GCAGA | |
| | CCu′ACAACCAGCu′Gu′u′CGAGGAGAACCCCAu′CAACGCCAG | |
| | CGGCGu′GGACGCCAAGGCCAu′CCu′GAGCGCCCGGCu′GAGC | |
| | AAGAGCCGGCGGCu′GGAGAACCu′GAu′CGCCCAGCu′GCCCG | |
| | GCGAGAAGAAGAACGGCCu′Gu′u′CGGCAACCu′GAu′CGCCCu′ | |
| | GAGCCu′GGGCCu′GACCCCCAACu′u′CAAGAGCAACu′u′CGAC | |
| | Cu′GGCCGAGGACGCCAAGCu′GCAGCu′GAGCAAGGACACCu′ | |
| | ACGACGACGACCu′GGACAACCu′GCu′GGCCCAGAu′CGGCGA | |
| | CCAGu′ACGCCGACCu′Gu′u′CCu′GGCCGCCAAGAACCu′GAGC | |
| | GACGCCAu′CCu′GCu′GAGCGACAu′CCu′GCGGGu′GAACACCG | |
| | AGAu′CACCAAGGCCCCCCu′GAGCGCCAGCAu′GAu′CAAGCG | |
| | Gu′ACGACGAGCACCACCAGGACCu′GACCCu′GCu′GAAGGCC | |
| | Cu′GGu′GCGGCAGCAGCu′GCCCGAGAAGu′ACAAGGAGAu′Cu′ | |
| | u′Cu′u′CGACCAGAGCAAGAACGGCu′ACGCCGGCu′ACAu′CGA | |
| | CGGCGGCGCCAGCCAGGAGGAGu′u′Cu′ACAAGu′u′CAu′CAAG | |
| | CCCAu′CCu′GGAGAAGAu′GGACGGCACCGAGGAGCu′GCu′GG | |
| | u′GAAGCu′GAACCGGGAGGACCu′GCu′GCGGAAGCAGCGGAC | |
| | Cu′u′CGACAACGGCAGCAu′CCCCCACCAGAu′CCACCu′GGGC | |
| | GAGCu′GCACGCCAu′CCu′GCGGCGGCAGGAGGACu′u′Cu′ACC | |
| | CCu′u′CCu′GAAGGACAACCGGGAGAAGAu′CGAGAAGAu′CCu′ | |
| | GACCu′u′CCGGAu′CCCCu′ACu′ACGu′GGGCCCCCu′GGCCCGG | |
| | GGCAACAGCCGGu′u′CGCCu′GGAu′GACCCGCAAGAGCGAGG | |
| | AGACAAu′CACCCCCu′GGAACu′u′CGAGGAGGu′GGu′GGACAA | |
| | GGGCGCCAGCGCCCAGAGCu′u′CAu′CGAGCGGAu′GACCAACu | |
| | ′u′CGACAAGAACCu′GCCCAACGAGAAGGu′GCu′GCCCAAGCA | |
| | CAGCCu′GCu′Gu′ ACGAGu′ACu′u′CACCGu′Gu′ACAACGAGCu′ | |
| | GACCAAGGu′GAAGu′ACGu′GACCGAGGGCAu′GCGGAAGCCC | |
| | GCCu′u′CCu′GAGCGGCGAGCAGAAGAAGGCCAu′CGu′GGACC | |
| | u′GCu′Gu′u′CAAGACCAACCGGAAGGu′GACCGu′GAAGCAGCu′ | |
| | GAAGGAGGACu′ACu′u′CAAGAAGAu′CGAGu′GCu′u′CGACAG | |
| | CGu′GGAGAu′CAGCGGCGu′GGAGGACCGGu′u′CAACGCCAGC | |
| | Cu′GGGCACCu′ACCACGACCu′GCu′GAAGAu′CAu′CAAGGACA | |
| | AGGACu′u′CCu′GGACAACGAGGAGAACGAGGACAu′CCu′GGA | |
| | GGACAu′CGu′GCu′GACCCu′GACCCu′Gu′u′CGAGGACCGGGAG | |
| | Au′GAu′CGAGGAGCGGCu′GAAGACCu′ACGCCCACCu′Gu′u′CG | |
| | ACGACAAGGu′GAu′GAAGCAGCu′GAAGCGGCGGCGGu′ACAC | |
| | CGGCu′GGGGCCGGCu′GAGCCGGAAGCu′GAu′CAACGGCAu′C | |
| | CGGGACAAGCAGAGCGGCAAGACCAu′CCu′GGACu′u′CCu′CA | |
| | AGAGCGACGGCu′u′CGCCAACCGGAACu′u′CAu′GCAGCu′GAu′ | |
| | CCACGACGACAGCCu′GACCu′u′CAAGGAGGACAu′CCAGAAG | |
| | GCCCAGGu′GAGCGGCCAGGGCGACAGCCu′GCACGAGCACAu | |
| | ′CGCCAACCu′GGCCGGCAGCCCCGCCAu′CAAGAAGGGCAu′C | |
| | Cu′GCAGACCGu′GAAGGu′GGu′GGACGAGCu′GGu′GAAGGu′G | |
| | Au′GGGCCGGCACAAGCCCGAGAACAu′CGu′GAu′CGAGAu′GG | |
| | CCCGGGAGAACCAGACCACCCAGAAGGGCCAGAAGAACAGC | |
| | CGGGAGCGGAu′GAAGCGGAu′CGAGGAGGGCAu′CAAGGAGC | |
| | u′GGGCAGCCAGAu′CCu′GAAGGAGCACCCCGu′GGAGAACAC | |
| | CCAGCu′GCAGAACGAGGCCCu′Gu′ACCu′Gu′ACu′ACCu′GCAG | |
| | AACGGCCGGGACAu′Gu′ACGu′GGACCAGGAGCu′GGACAu′CA | |
| | ACCGGCu′GAGCGACu′ACGACGu′GGACCACAu′CGu′GCCCCA | |
| | GAGCu′u′CCu′GAAGGACGACAGCAu′CGACAACAAGGu′GCu′G | |
| | ACCCGGAGCGACAAGAACCGGGGCAAGAGCGACAACGu′GCC | |
| | CAGCGAGGAGGu′GGu′GAAGAAGAu′GAAGAACu′ACu′GGCG | |
| | GCAGCu′GCu′GAACGCCAAGCu′GAu′CACCCAGCGGAAGu′u′C | |
| | GACAACCu′GACCAAGGCCGAGCGGGGCGGCCu′GAGCGAGCu | |
| | ′GGACAAGGCCGGCu′u′CAu′CAAGCGGCAGCu′GGu′GGAGAC | |
| | ACGGCAGAu′CACCAAGCACGu′GGCCCAGAu′CCu′GGACAGC | |
| | CGGAu′GAACACCAAGu′ACGACGAGAACGACAAGCu′GAu′CC | |
| | GGGAGGu′GAAGGu′GAu′CACCCu′CAAGAGCAAGCu′GGu′GA | |
| | GCGACu′u′CCGGAAGGACu′u′CCAGu′u′Cu′ACAAGGu′GCGGG | |
| | AGAu′CAACAACu′ACCACCACGCCCACGACGCCu′ACCu′GAAC | |
| | GCCGu′GGu′GGGCACCGCCCu′GAu′CAAGAAGu′ACCCCGCCC | |
| | u′GGAGAGCGAGu′u′CGu′Gu′ACGGCGACu′ACAAGGu′Gu′ACG | |
| | ACGu′GCGGAAGAu′GAu′CGCCAAGAGCGAGCAGGAGAu′CGG | |
| | CAAGGCCACCGCCAAGu′ACu′u′Cu′u′Cu′ACAGCAACAu′CAu′G | |
| | AACu′u′Cu′u′CAAGACCGAGAu′CACCCu′GGCCAACGGCGAGA | |
| | u′CCGGAAGGCCCCCCu′GAu′CGAGACAAACGGCGAGACAGG | |
| | CGAGAu′CGu′Gu′GGGACAAGGGCCGGGACu′u′CGCCACCGu′G | |
| | CGGAAGGu′GCu′GAGCAu′GCCCCAGGu′GAACAu′CGu′GAAGA | |
| | AGACCGAGGu′GCAGACCGGCGGCu′u′CAGCAAGGAGAGCAu′ | |
| | CCu′GCCCAAGCGGAACAGCGACAAGCu′GAu′CGCCCGGAAG | |
| | AAGGACu′GGGACCCCAAGAAGu′ACGGCGGCu′u′CGACAGCC | |
| | CCACCGu′GGCCu′ACAGCGu′GCu′GGu′GGu′GGCCAAGGu′GGA | |
| | GAAGGGCAAGAGCAAGAAGCu′CAAGAGCGu′GAAGGAGCu′G | |
| | Cu′GGGCAu′CACCAu′CAu′GGAGCGGAGCAGCu′u′CGAGAAGA | |
| | ACCCCAu′CGACu′u′CCu′GGAGGCCAAGGGCu′ACAAGGAGGu′ | |
| | GAAGAAGGACCu′GAu′CAu′CAAGCu′GCCCAAGu′ACAGCCu′G | |
| | u′u′CGAGCu′GGAGAACGGCCGGAAGCGGAu′GCu′GGCCAGCG | |
| | CCGGCGAGCu′GCAGAAGGGCAACGAGCu′GGCCCu′GCCCAGC | |
| | AAGu′ACGu′GAACu′u′CCu′Gu′ACCu′GGCCAGCCACu′ACGAGA | |
| | AGCu′GAAGGGCAGCCCCGAGGACAACGAGCAGAAGCAGCu′ | |
| | Gu′u′CGu′GGAGCAGCACAAGCACu′ACCu′GGACGAGAu′CAu′C | |
| | GAGCAGAu′CAGCGAGu′u′CAGCAAGCGGGu′GAu′CCu′GGCCG | |
| | ACGCCAACCu′GGACAAGGu′GCu′GAGCGCCu′ACAACAAGCA | |
| | CCGGGACAAGCCCAu′CCGGGAGCAGGCCGAGAACAu′CAu′CC | |
| | ACCu′Gu′u′CACCCu′GACCAACCu′GGGCGCCCCCGCCGCCu′u′ | |
| | CAAGu′ACu′u′CGACACCACCAu′CGACCGGAAGCGGu′ACACC | |
| | AGCACCAAGGAGGu′GCu′GGACGCCACCCu′GAu′CCACCAGA | |
| | GCAu′CACCGGCCu′Gu′ACGAGACACGGAu′CGACCu′GAGCCA | |
| | GCu′GGGCGGCGACGAGGGCGCCGACAAGCGGACCGCCGACG | |
| | GCAGCGAGu′u′CGAGAGCCCCAAGAAGAAGCGGAAGGu′Gu′G | |
| | A | |
| 3′ | GCGGCCGCu′u′AAu′u′AAGCu′GCCu′u′Cu′GCGGGGCu′u′GCCu′ | 700 |
| UTR | u′Cu′GGCCAu′GCCCu′u′Cu′u′Cu′Cu′CCCu′u′GCACCu′Gu′ACCu′ | |
| | Cu′u′GGu′Cu′u′u′GAAu′AAAGCCu′GAGu′AGGAAGu′Cu′AGA | |
| | MSEVEFSHEYWMRHALTLAKRARDEREVPVGAVLVLNNRVIGE | 730 |
| MA067 | GWNRAIGLHDPTAHAEIMALRQGGLVMQNYRLIDATLYVTFEP | |
| amino | CVMCAGAMIHSRIGRVVFGVRNAKTGAAGSLMDVLHHPGMNH | |
| acid | RVEITEGILADECAALLCRFFRMPRRVFNAQKKAQSSTDSGGSSG | |
| sequence | GSSGSETPGTSESATPESSGGSSGGSDKKYSIGLAIGTNSVGWAVI | |
| | TDEYKVPSKKFKVLGNTDRHSIKKNLIGALLFDSGETAEATRLK | |
| | RTARRRYTRRKNRICYLQEIFSNEMAKVDDSFFHRLEESFLVEED | |
| | KKHERHPIFGNIVDEVAYHEKYPTIYHLRKKLVDSTDKADLRLIY | |
| | LALAHMIKFRGHFLIEGDLNPDNSDVDKLFIQLVQTYNQLFEENP | |
| | INASGVDAKAILSARLSKSRRLENLIAQLPGEKKNGLFGNLIALSL | |
| | GLTPNFKSNFDLAEDAKLQLSKDTYDDDLDNLLAQIGDQYADL | |
| | FLAAKNLSDAILLSDILRVNTEITKAPLSASMIKRYDEHHQDLTL | |
| | LKALVRQQLPEKYKEIFFDQSKNGYAGYIDGGASQEEFYKFIKPI | |
| | LEKMDGTEELLVKLNREDLLRKQRTFDNGSIPHQIHLGELHAILR | |
| | RQEDFYPFLKDNREKIEKILTFRIPYYVGPLARGNSRFAWMTRKS | |
| | EETITPWNFEEVVDKGASAQSFIERMTNFDKNLPNEKVLPKHSLL | |
| | YEYFTVYNELTKVKYVTEGMRKPAFLSGEQKKAIVDLLFKTNR | |
| | KVTVKQLKEDYFKKIECFDSVEISGVEDRFNASLGTYHDLLKIIK | |
| | DKDFLDNEENEDILEDIVLTLTLFEDREMIEERLKTYAHLFDDKV | |
| | MKQLKRRRYTGWGRLSRKLINGIRDKQSGKTILDFLKSDGFANR | |
| | NFMQLIHDDSLTFKEDIQKAQVSGQGDSLHEHIANLAGSPAIKK | |
| | GILQTVKVVDELVKVMGRHKPENIVIEMARENQTTQKGQKNSR | |
| | ERMKRIEEGIKELGSQILKEHPVENTQLQNEALYLYYLQNGRDM | |
| | YVDQELDINRLSDYDVDHIVPQSFLKDDSIDNKVLTRSDKNRGK | |
| | SDNVPSEEVVKKMKNYWRQLLNAKLITQRKFDNLTKAERGGLS | |
| | ELDKAGFIKRQLVETRQITKHVAQILDSRMNTKYDENDKLIREV | |
| | KVITLKSKLVSDFRKDFQFYKVREINNYHHAHDAYLNAVVGTA | |
| | LIKKYPALESEFVYGDYKVYDVRKMIAKSEQEIGKATAKYFFYS | |
| | NIMNFFKTEITLANGEIRKAPLIETNGETGEIVWDKGRDFATVRK | |
| | VLSMPQVNIVKKTEVQTGGFSKESILPKRNSDKLIARKKDWDPK | |
| | KYGGFDSPTVAYSVLVVAKVEKGKSKKLKSVKELLGITIMERSS | |
| | FEKNPIDFLEAKGYKEVKKDLIIKLPKYSLFELENGRKRMLASAG | |
| | ELQKGNELALPSKYVNFLYLASHYEKLKGSPEDNEQKQLFVEQ | |
| | HKHYLDEIIEQISEFSKRVILADANLDKVLSAYNKHRDKPIREQA | |
| | ENIIHLFTLTNLGAPAAFKYFDTTIDRKRYTSTKEVLDATLIHQSI | |
| | TGLYETRIDLSQLGGDEGADKRTADGSEFESPKKKRKV | |

Chemistry and modification used MA040, MA041, MA045, MA064 MA065, MA066, and MA067 mRNAs:
-u′ = N¹-methyl pseudouridine
-The first nucleotide in the 5′ UTR has a 2′-O-methyl modification
-The first nucleotide in the 5′ UTR is linked via a 5′-5′ triphosphate bridge to a 7-methylguanosine nucleoside
-mRNAs have a 3′ poly A region

### Example 8. gRNA Variants

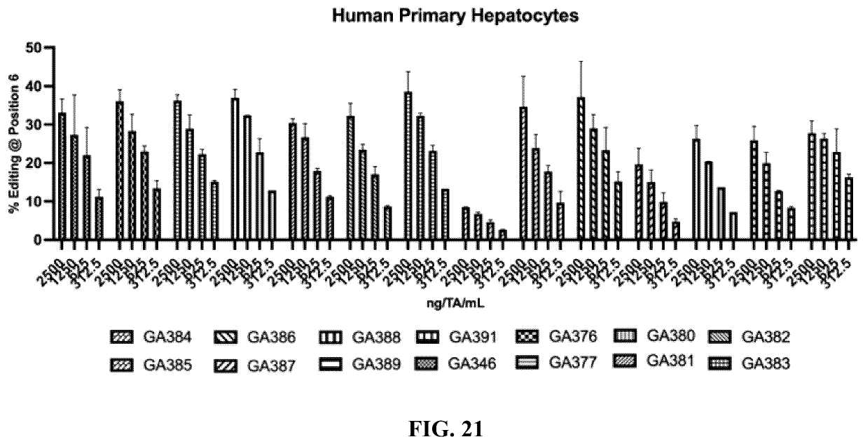

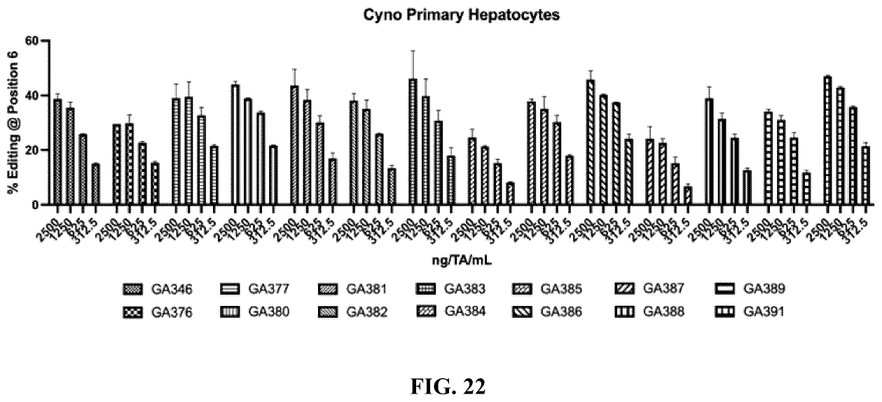

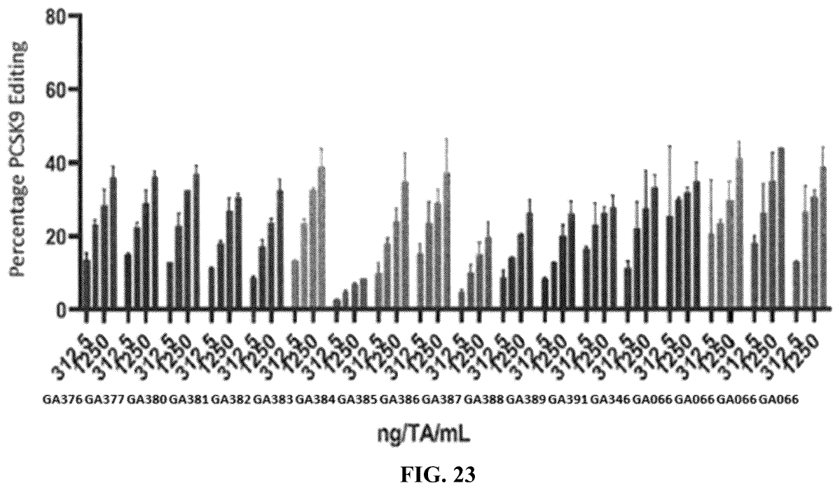

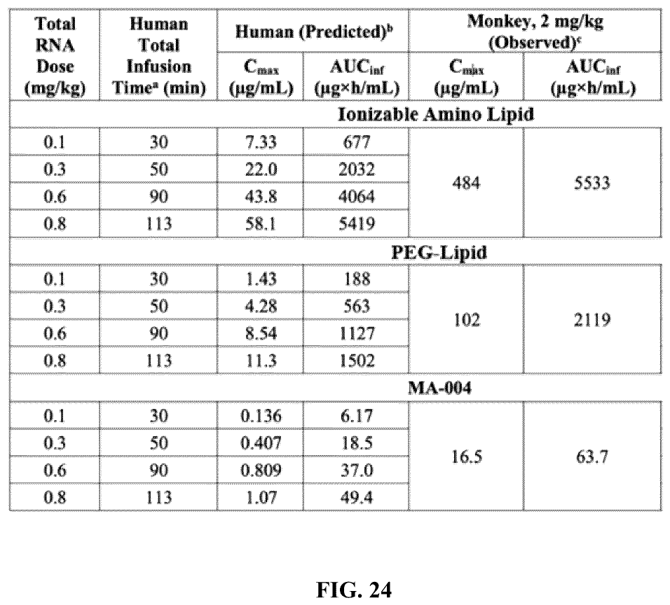

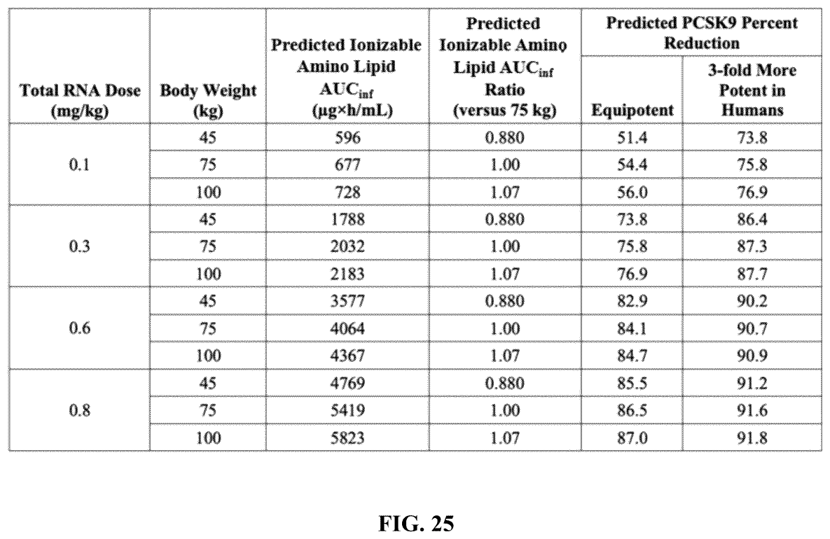

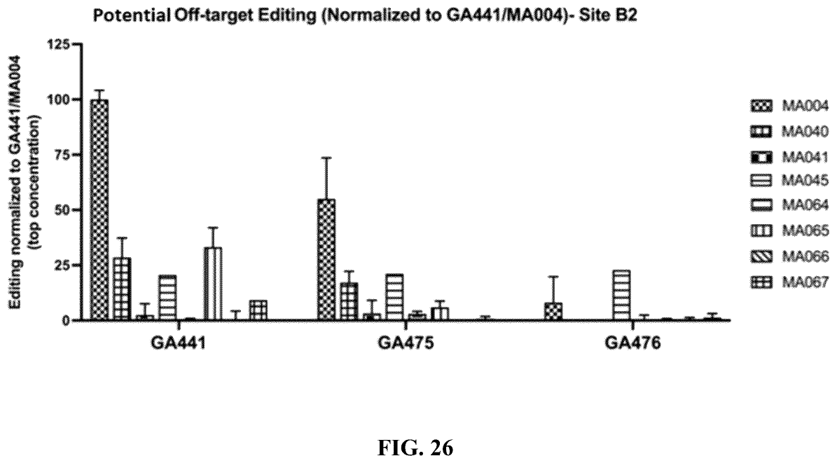

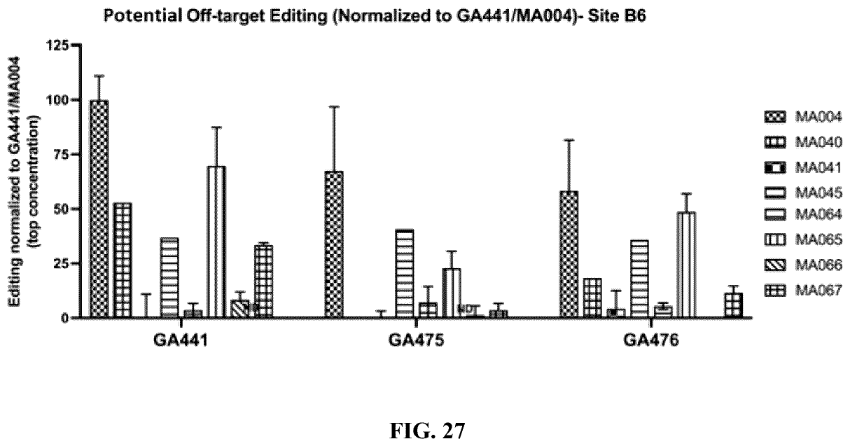

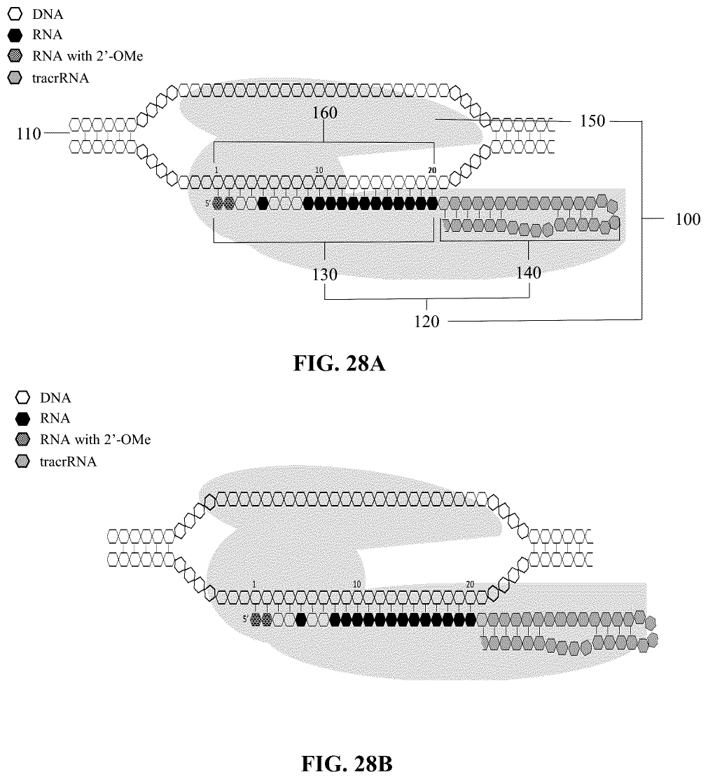

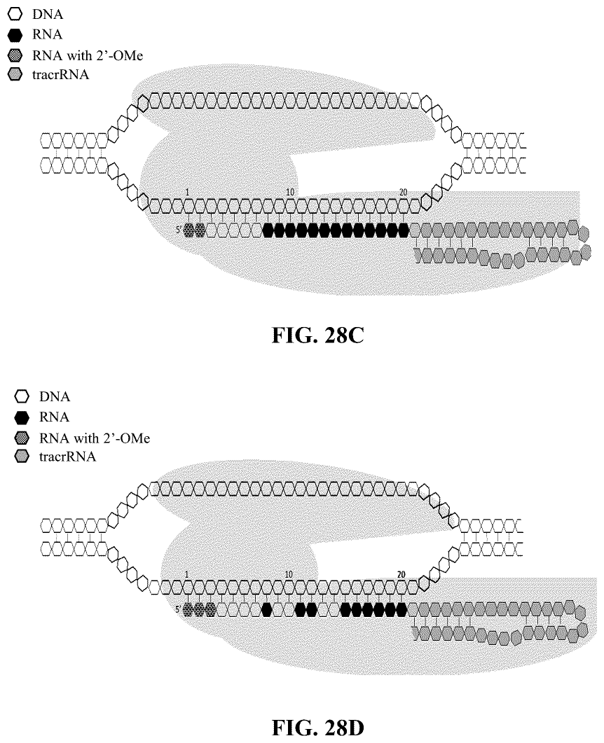

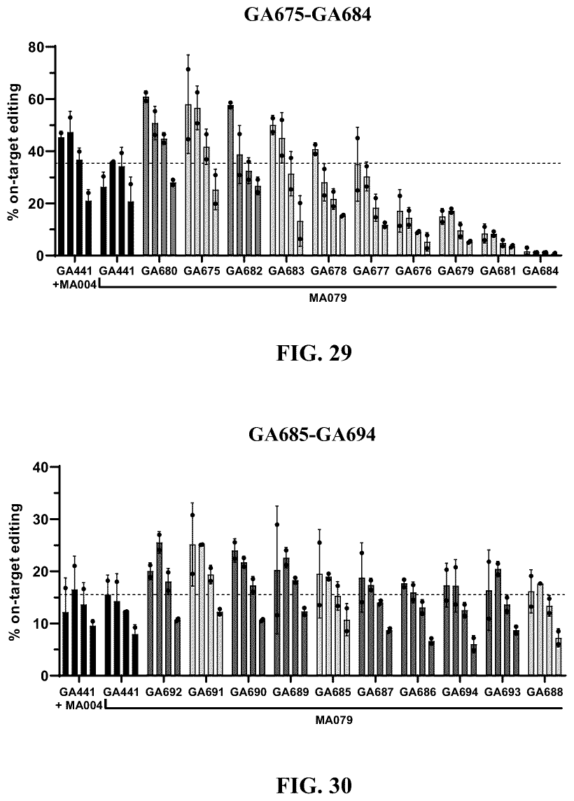

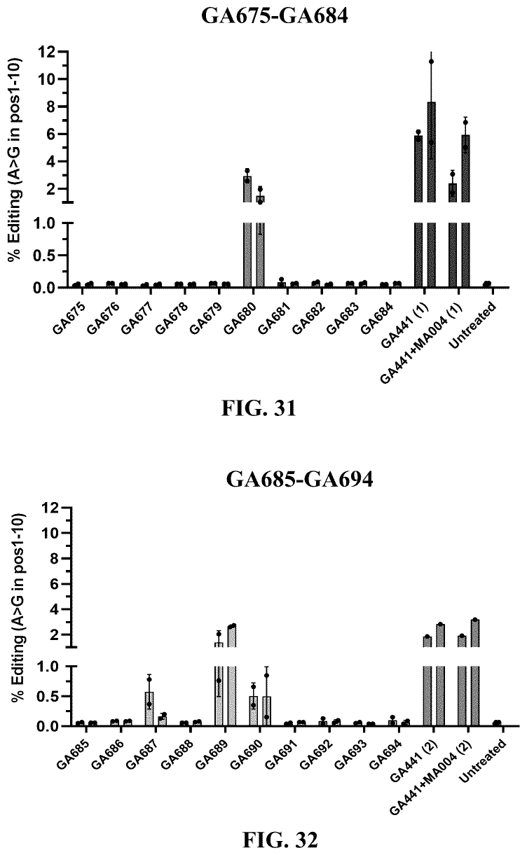

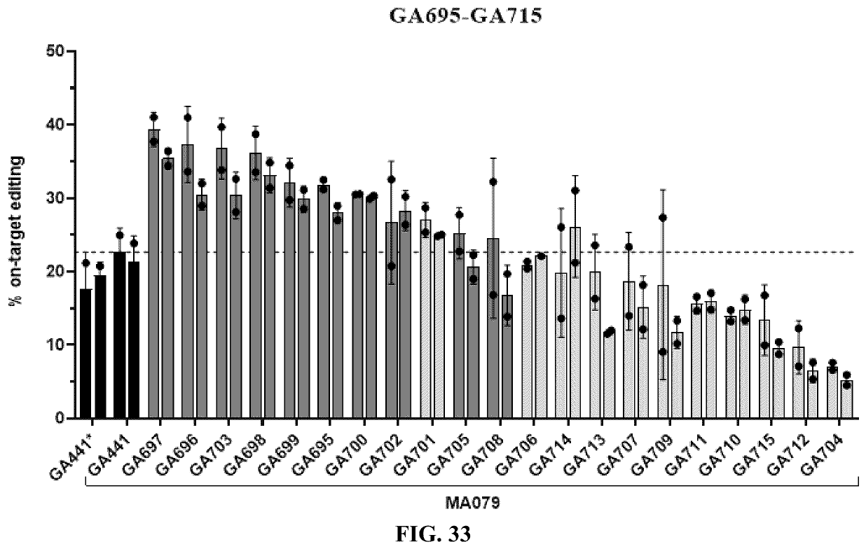

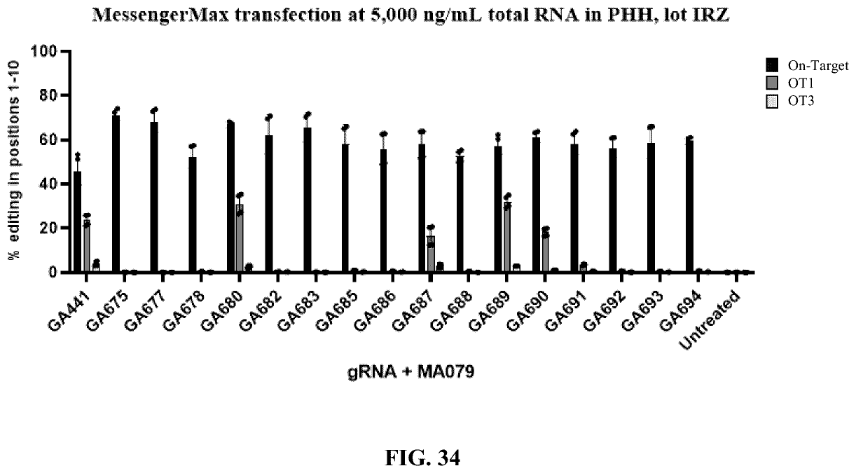

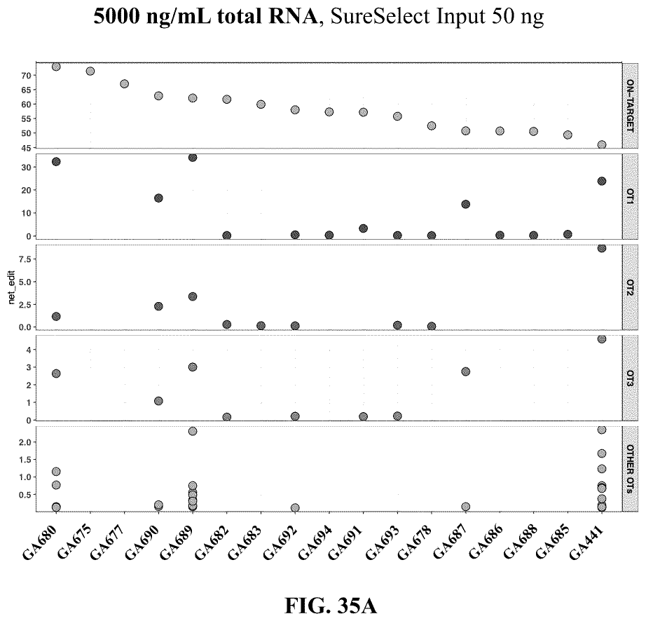

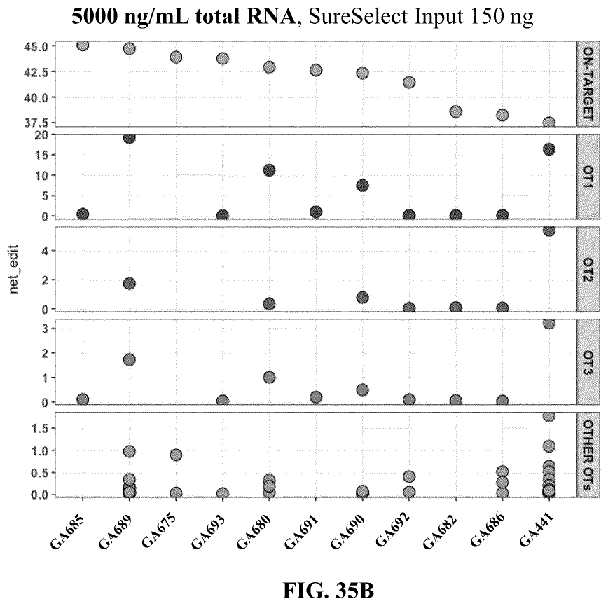

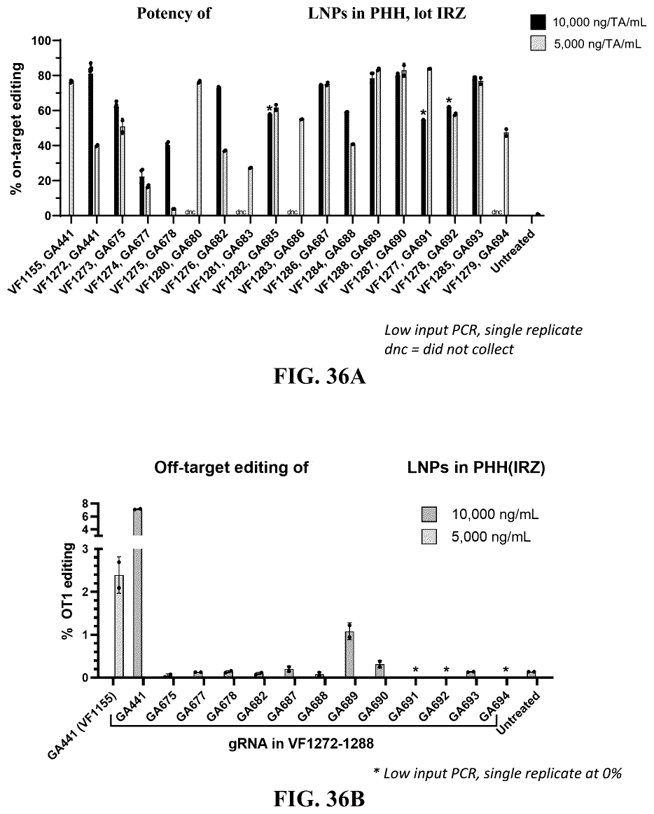

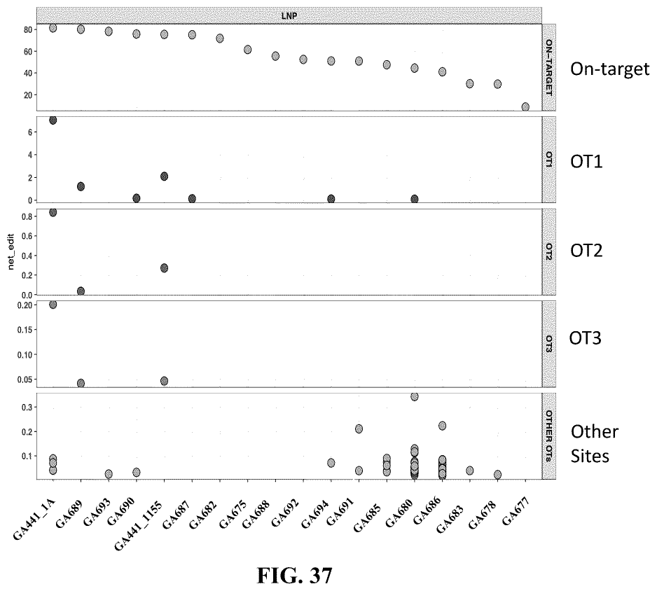

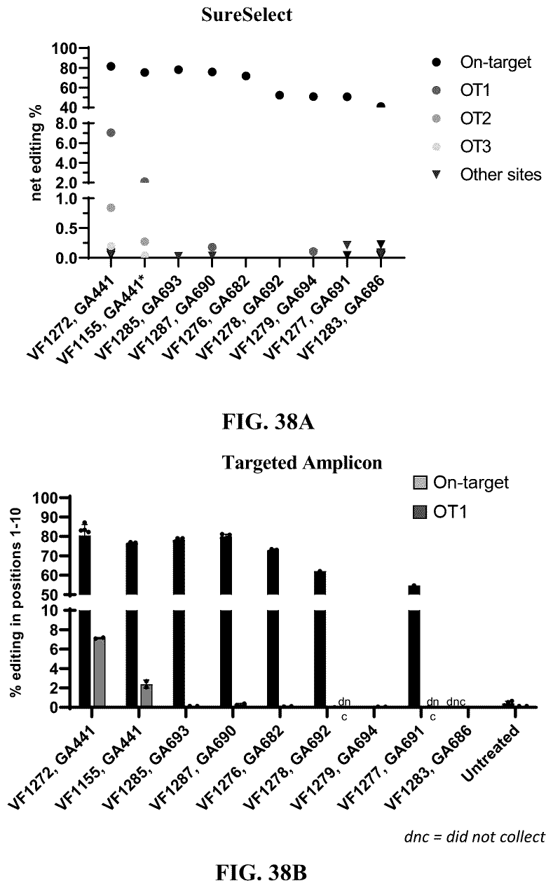

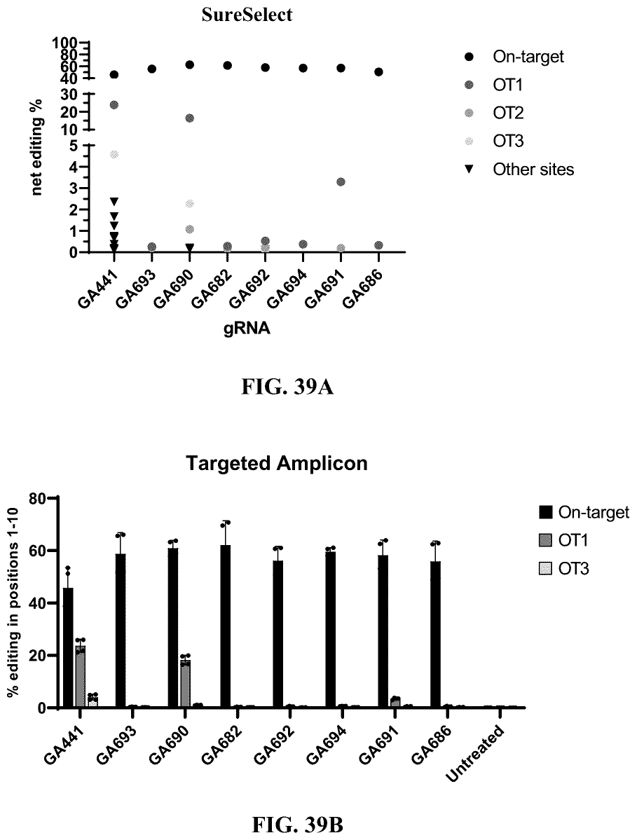

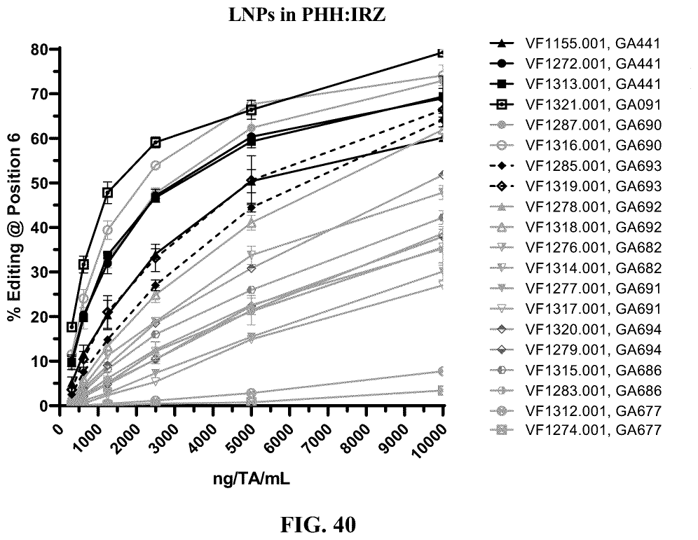

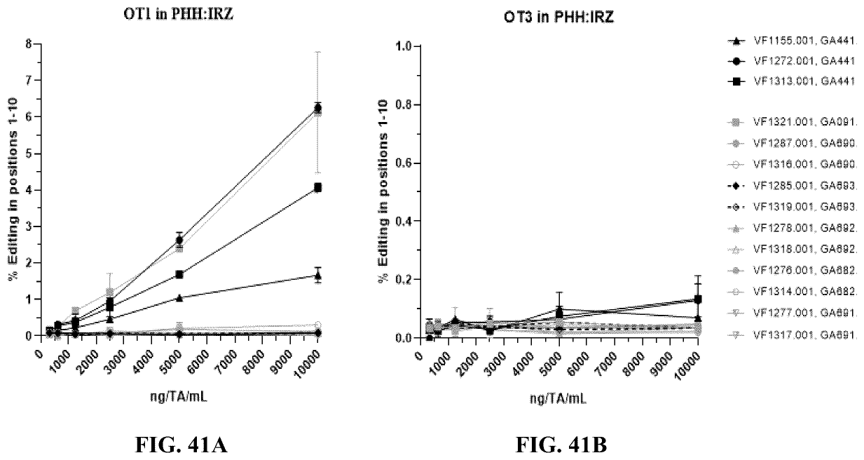

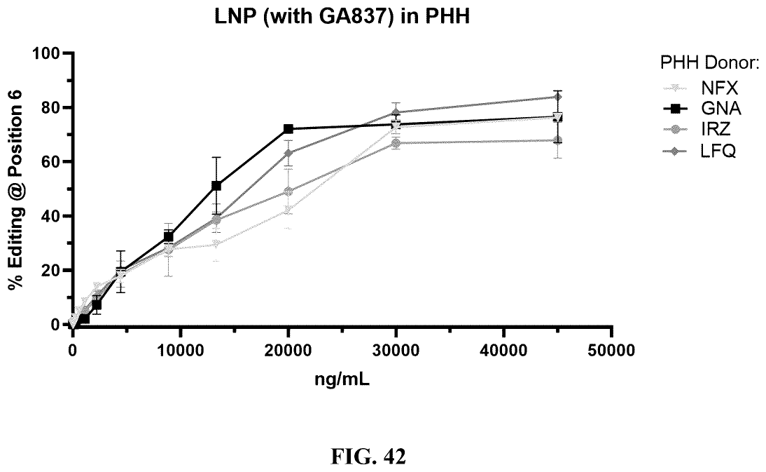

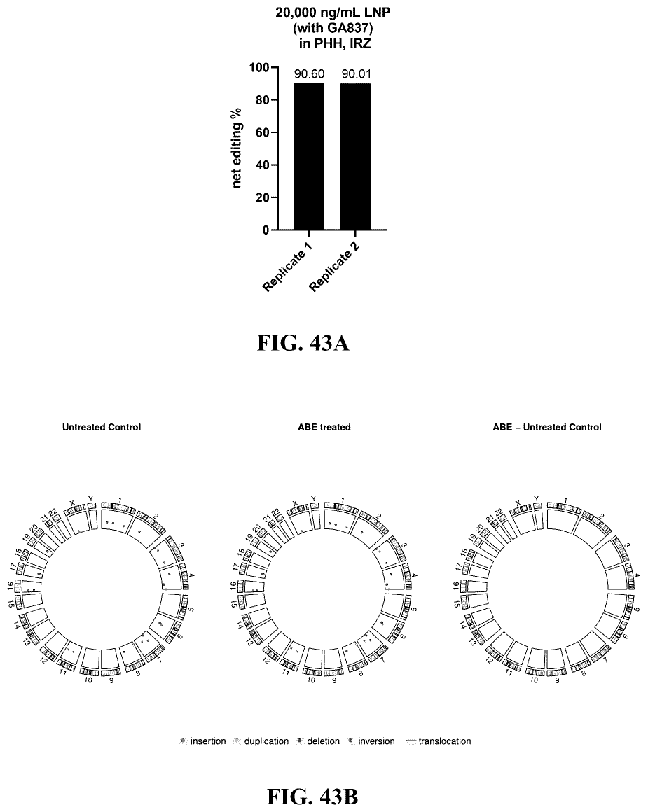

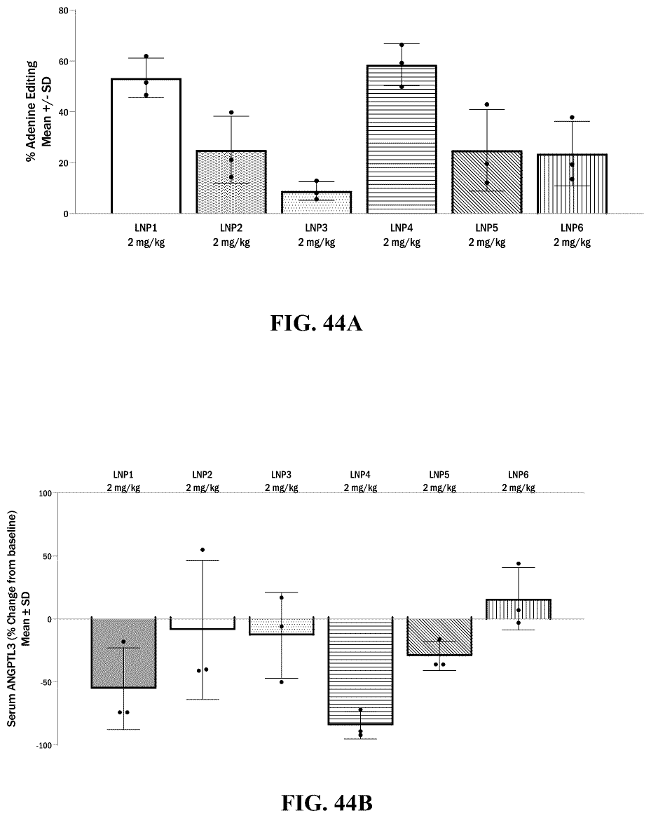

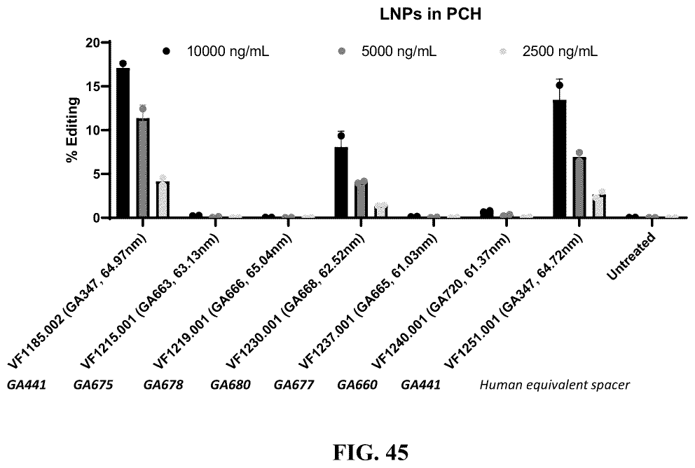

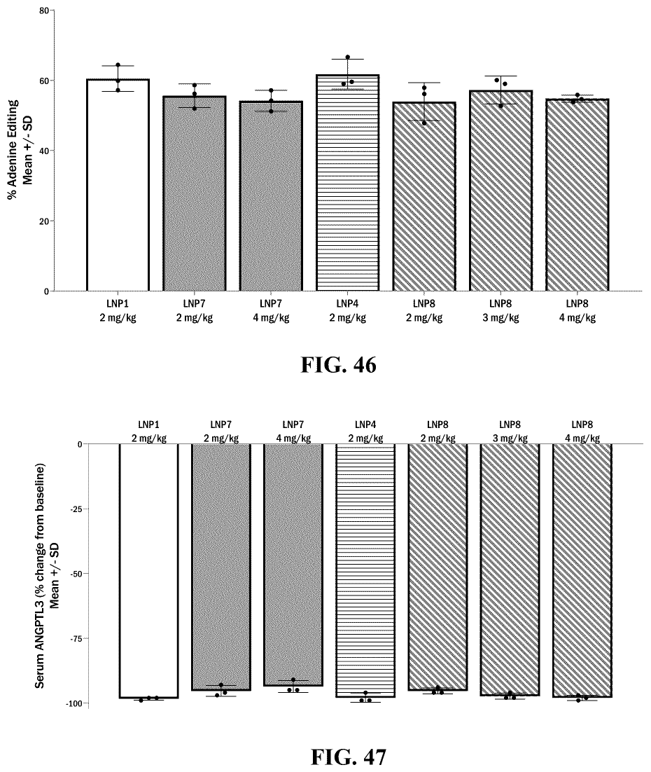

Exemplary PCSK9 guide RNA variants are shown in Table 3. Additional exemplary PCSK9 guide RNA variants can be found in PCT Application Publication NO. WO2021/207712, which is incorporated herein by reference in its entirety. The on-target editing efficiency of those PCSK9 guides are shown in FIGS. 21-23.

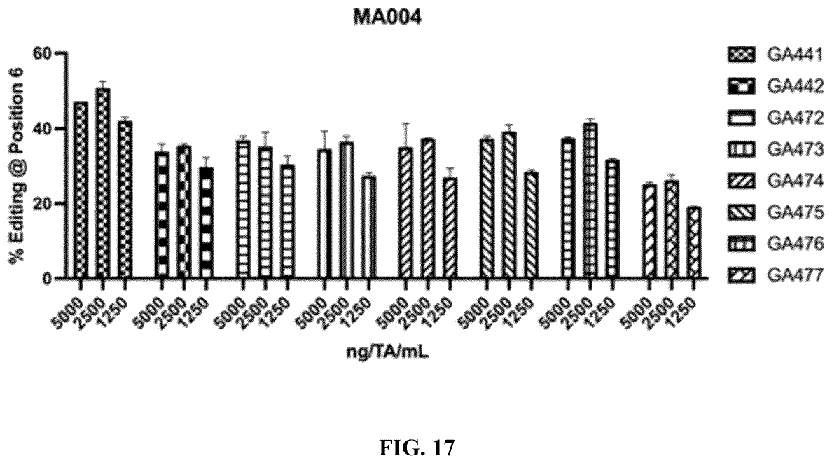

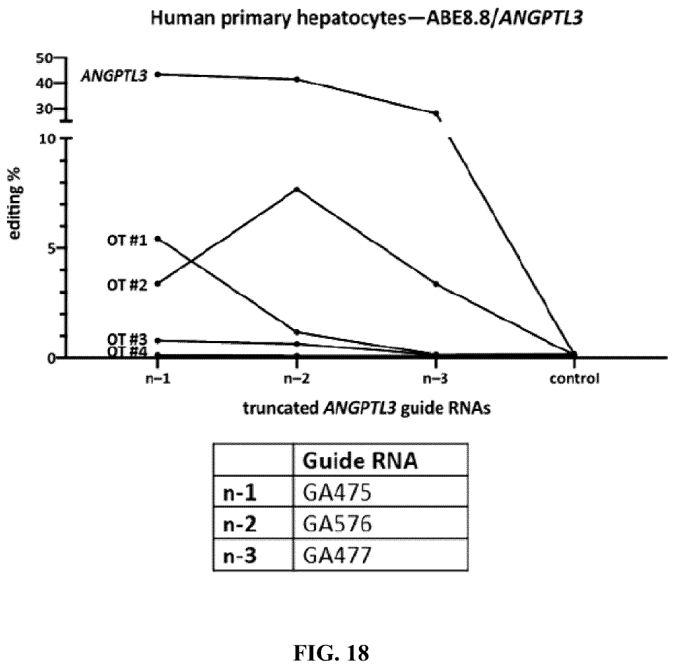

Exemplary ANGPTL3 guide RNA variants are shown in Table 3 and Table 8. Additional exemplary ANGPTL3 guide RNA variants can be found in PCT Application Publication NO. WO2021/207712, which is incorporated herein by reference in its entirety. The on-target editing efficiency of the variant ANGPTL3 guides are shown in FIG. 17 and FIG. 18.

### Assessment of Proliferation of Edited Cells (HuH-7s):

As previously described, In vitro efficacy of exemplary gRNAs and ABE mRNAs was evaluated by transfecting the cells and with the specified concentrations, collecting the cells after transfection (3 days) and analyzing the editing percentage using PCR and sequencing as previously described.

TABLE 3
GA347 Protospacer with Nebularine (E) and Inosine (I/i) modifications

| | | | SEQ | | SEQ |
|---|---|---|---|---|---|
| Gene | gRNA | Protospacer | ID | | ID |
| Target | ID | (5′-3′) | NO | Sequence (5′-3′) | NO. |
| CANG | GA539 | AAGATAC | 177 | asasgsAUXCCUGAAUAACCCUCgUUU | 757 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA540 | AAGATAC | 177 | asasgsAUXCCUGXAUAACCCUCgUUU | 758 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA541 | AAGATAC | 177 | asasgsAUXCCUGAAUAXCCCUCgUUU | 759 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA542 | AAGATAC | 177 | asasgsAUXCCUGXAUXACCCUCgUUU | 760 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA543 | AAGATAC | 177 | asasgsXUXCCUGAAUAACCCUCgUUU | 761 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA544 | AAGATAC | 177 | asasgsXUXCCUGAAUAXCCCUCgUUU | 762 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| cANG | GA545 | AAGATAC | 177 | asasgsXUACCUGAAUXACCCUCgUUU | 763 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA546 | AAGATAC | 177 | asasgsAUACCUGXAUAACCCUCgUUU | 764 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA548 | AAGATAC | 177 | asasgsAUACCUGEAUAXCCCUCgUUU | 765 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA549 | AAGATAC | 177 | asasgsAUXCCUGAAUAXCCCUCgUUU | 759 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA613 | AAGATAC | 177 | asasisAUACCUGAAUAACUCUCgUUU | 766 |
| PTL3 | | CTGAATA | | UAGagcuaGaaauagcaaGUUaAaAuAaggcu | |
| | | ACTCTC | | aGUccGUUAucAAcuuGaaaaagugGcaccga | |
| | | | | gucggugcusususu | |
| CANG | GA614 | AAGATAC | 177 | asasisAUACCUIAAUAACUCUCgUUUU | 767 |
| PTL3 | | CTGAATA | | AGagcuaGaaauagcaaGUUaAaAuAaggcua | |
| | | ACTCTC | | GUccGUUAucAAcuuGaaaaagugGcaccgag | |
| | | | | ucggugcusususu | |

Uppercase nucleotides (A, G, C, I and T) indicate 2′-deoxyribonucleotides, adenine, guanine, cytosine, inosine, and thymine, respectively; in Guide RNA sequence: uppercase nucleotides (A, C, G, I and U) indicate ribonucleotides, adenine, guanine, cytosine, inosine, and uracil, respectively and lowercase nucleotides (a, g, c, i and u) indicate 2′-O-methylribonucleotide (2′-OMe) unless otherwise specified.
X is nebularine.
It is understood that the DNA protospacer is converted to RNA or RNA-equivalent in the guide RNA
s: phosphorothioate (PS).

### Example 9. Human Dosing (ABE MA004, GA346, 1:1 mRNA:RNA Ratio by Weight Encapsulated in an LNP Comprising Ionizable Amino Lipid and PEG-Lipid)

The human pharmacokinetic (PK) parameters (i.e., clearance [CL] and volume of distribution at steady state [Vss]) for Ionizable Amino Lipid, PEG-Lipid, and MA-004 were predicted based on the PK parameters in cynomolgus monkeys using allometric scaling with 0.75 and 1 as the exponent for CL and Vss, respectively. The cynomolgus monkeys were dosed with the total RNA dose ranging from 0.5 to 2.5 mg/kg; within this dose range, the PK parameters were relatively constant. The concentration versus time profiles in humans following different total RNA doses using graded infusions were simulated using a one-compartmental model and Berkeley Madonna® Version 8.3.18 was used for the simulations. The predicted plasma maximum concentration (Cmax) in humans were obtained based on the concentration versus time profiles. The area under the plasma concentration-time curve from time zero to infinity (AUCinf) predicted in humans was calculated as the ratio of the analyte dose over the predicted CL value.

The relationship between Ionizable Amino Lipid AUCinf (after a single dose) and PCSK9% reduction at steady state in cynomolgus monkeys was analyzed using an inhibitory Imax function, and R Software Version 3.6.1 and RStudio Version 1.2.5001 were used for the fitting and plotting. Based on the inhibitory Imax function, Ionizable Amino Lipid AUCinf value associated with 50% of maximal inhibition (AUC50) in cynomolgus monkeys was estimated. This AUC50 value was used to predict human PCSK9 reduction assuming an ABE mRNA MA004 and guide RNA GA346 is equipotent between cynomolgus monkeys and humans. Alternatively, this AUC50 value was divided by 3 and then used to predict human PCSK9 reduction assuming an ABE mRNA MA004 and guide RNA GA346 is 3-fold more potent in humans than in cynomolgus monkeys based on the in vitro testing results of an ABE mRNA MA004 and guide RNA GA346 in primary cynomolgus monkey hepatocytes (PCH) and primary human hepatocytes (PHH). With either scenario (equipotency or 3-fold difference in potency), Ionizable Amino Lipid AUCinf values predicted in humans were used to predict PCSK9% reduction at steady state following different total RNA doses.

Ionizable Amino Lipid AUC in the plasma was used as a surrogate of LNP constituted with an ABE mRNA MA004 and guide RNA GA346 exposure in the plasma and ultimately LNP constituted with an ABE mRNA MA004 and guide RNA GA346 exposure in the liver where the editing takes place.

The relationship between Ionizable Amino Lipid exposure (area under the plasma concentration-time curve [AUC]) and PCSK9 percentage reduction was characterized in NHP. To estimate pharmacological activity in humans at various dose levels, Ionizable Amino Lipid exposure (AUC) in humans was predicted using allometric scaling and subsequently percentage of PCSK9 reduction was estimated. The tables in FIGS. 24 and 25 present the predicted Cmax, AUC and potency for humans at various doses. As noted therein the 0.1 mg/kg total RNA dose is estimated to exhibit approximately 54.4% to 75.8% PCSK9 reduction (considering a 1× to 3×potency factor) in a participant weighing 75 kg. The body weight effect (across the range of 45 kg to 100 kg) on PCSK9 was predicted to be minimal (with an absolute difference ≤3% relative to 75 kg) for a given mg/kg total RNA dose. As set forth in the table in FIG. 25, 0.8 mg/kg total RNA dose is estimated to exhibit approximately 86.5% to 91.8% PCSK9 reduction (considering a 1× to 3× potency factor) in a participant weighing 75 kg. The predicted Cmax and AUC levels relative to those observed in NHP models dosed at 2 mg/Kg are presented in the table in FIG. 24.

### Example 10. On-Target and Off-Target Editing Rates in Primary Human Hepatocytes (PHH)

This example illustrates the use of the guide nucleic acids comprising the spacer sequences (sgRDNA) to evaluate their on-targeting editing percentage of ANGPTL3 gene at either position 6 or positions 1-10 of protospacer region in the ANGPTL3 gene in PHH cells (FIGS. 29-30).

Twenty (20) sgRDNA spacer sequence variants with minimally modified tracrRNA (GA675-GA694, SEQ ID NOs: 72, 75, 79, 83, 86, 89, 93, 96, 98, 100, 102, 106, 110, 114, 116, 120, 124, 128, 132, 136, Table 7) were transfected into PHH cells (lot STL) together with mRNA MA079 according to the protocol of MessengerMax™. The term “minimal modified tracrRNA” refers to a tracrRNA of sequence GUUUUAGAGCUAGAAAUAGCAAGUUAAAAUAAGGCUAGUCCGUUAUCAACUUGA AAAAGUGGCACCGAGUCGGUGCUsususu (SEQ ID NO: 3). Control groups were (a) gRNA GA441 (2′-OMe-enriched tracrRNA) transfected with mRNA MA004, and (b) gRNA GA441 (2′-OMe-enriched tracrRNA) transfected with mRNA MA079. The term “2′-OMe-enriched tracrRNA” refers to a tracr of sequence of gUUUUAGagcuaGaaauagcaaGUUaAaAuAaggcuaGUccGUUAucAAcuuGaaaaagugGcaccgaguc ggugcusususu (SEQ ID NO: 4). The doses of the total RNA were 5,000 ng/mL, 2,500 ng/mL, 1,250 ng/mL, and 625 ng/mL. On-target primer (GAO100_OT_B1) was used to measure editing percentage (A>G) in position 1-10 of protospacer region in the ANGPTL3 gene by Targeted Amplicon Sequencing. Technical replicates were performed. N refers to any non-A nucleotide detected at this position in the sequencing data.

The genomic DNA from PHH cells transfected with one of 20 sgRDNA spacer sequence variants were also tested for off-target editing percentage by targeted amplicon sequencing (FIGS. 31-32). The genomic DNA samples assessed were from cells that received doses of the total RNA of 5,000 ng/mL and 2,500 ng/mL. OT1 primer (GA0100_OT_B2) was used to measure editing percentage (A>G) in position 1-10 of the OT1 site (Table. 4). Technical replicates were performed. The results suggest that majority of the sgRDNA spacer sequences was not binding to and/or editing at OT1. For the rest of the sgRDNAs comprising spacer sequences that indeed edited at OT1 (GA680, GA689, GA690, GA687), about 75% of them have only 2-3 DNA bases.

TABLE 4
Information Regarding the Off-target Site (OT1)

| | | VEP | | CADD | |
|---|---|---|---|---|---|
| Site ID | Alignment | Gene | VEP Consequence | score | COSMIC |
| Chr8_66947473_(+) | RNA21 | TCF24 | 3′UTR, | 6.92 | No |
| | | | downstream gene | | |

Table. 4 shows the information about the off-target site (OT1) assessed in the experiments. Alignment indicates how OT1 differs in sequence from the on-target protospacer sequence—there are 2 mismatches and 1 RNA bulge, which occurs when the genome sequence contains a deletion with respect to the crRNA sequence of the gRNA (i.e., there is a deficit of paired nucleotide in the genome sequence). VEP Gene and VEP Consequence refer to the genomic location and functional role of OT1 as determined by the Ensembl Variant Effect Predictor (VEP) tool v98.3. Ensembl VEP uses an extensive collection of genomic annotations, including transcript models, regulatory features, and variant data, for the human genome, to allow the analysis, annotation and interpretation of genomic variants, or in this case hypothetical editing, in both protein-coding and non-protein-coding regions of the genome. The CADD score was determined using the Combined Annotation-Dependent Depletion v1.6 (CADD) framework, which aims to annotate and interpret human genetic variation through the integration of diverse annotations into a single measure. The CADD score can be used to assess the functional impact of editing at off-target sites on cellular function; a Phred-scaled CADD score of 10 or greater indicates that an edit at that site is in the top 10% of likelihood of having a functional effect. Thus, the CADD score of 6.92 suggests that there is not a high likelihood that editing at OT1 will have a functional consequence. To bioinformatically assess the potential for oncogenesis due to editing at the off-target site, it was assessed whether OT1 is located within a gene in the Cancer Gene Census of the Catalogue of Somatic Mutations in Cancer (COSMIC) v92; it is not.

Additional 21 sgRDNA spacer sequence variants with minimally modified tracrRNA (GA695-GA715, Table 7) were transfected into PHH cells (lot STL) together with mRNA MA079 according to the protocol of MessengerMax™. Control groups were (a) gRNA GA441 (2′-OMe-enriched tracrRNA) transfected with mRNA MA004, and (b) gRNA GA441 (2′-OMe-enriched tracrRNA) transfected with mRNA MA079. The doses of the total RNA were 5,000 ng/mL and 2,500 ng/mL. On-target primer (GA0100_OT_B1) was used to measure editing percentage (A>N) in position 6 of the protospacer region in the ANGPTL3 gene by Targeted Amplicon sequencing. Technical replicates were performed (FIG. 33 illustrates the data of this study).

Sixteen (16) sgRDNA spacer sequence variants with minimally modified tracrRNA (GA675, GA677, GA678, GA680, GA682, GA683, GA685, GA686, GA687, GA688, GA689, GA690, GA691, GA692, GA693, GA694) were selected and transfected into PHH cells (lot IRZ) together with mRNA MA079 according to the protocol of MessengerMax™. Control groups were gRNA GA441 (2′-OMe-enriched tracrRNA) transfected with mRNA MA079. The dose of the total RNA was 5,000 ng/mL. On-target primer (GA0100_OT_B1) was used to measure editing percentage (A>G) in positions 1-10 of protospacer region in the ANGPTL3 gene. OT1 primer (GA0100_OT_B2) and OT3 primer (GA0100_OT_B6) were used to measure off-target editing percentage (A>G) in positions 1-10 of the off-target sites. Technical replicates were performed (FIG. 34 presents data from this study).

The genomic DNA from PHH cells transfected with 16 sgRDNA spacer sequence variants were further tested for off-target editing according to the protocol of SureSelect (FIGS. 35A-35B). For FIG. 35A, the input DNA for SureSelect assay was 50 ng, and a panel of RNA probes targeting 611 candidate off-target sites derived from ONE-seq with GA441 was used. For FIG. 35B, Targeted Amplicon was not used for assessment independently. In a separate experiment, 10 top sgRNA spacer sequence variants with minimally modified tracrRNA were selected and transfected with MA079 in PHH cells (lot IRZ) according to the protocol of MessengerMax™. Control group was gRNA GA441 (2′-OMe-enriched tracrRNA) transfected with MA079. The input DNA for SureSelect assay was 150 ng, and a panel of RNA probes targeting 611 candidate off-target sites derived from ONE-seq with GA441 was used. The plot shows net editing percentage of significant sites (non-adjusted pᵥₐₗ<0.05) and ordered by on-target potency.

Sixteen (16) sgRDNA comprising the spacer sequence variants with minimally modified tracrRNA were selected and transfected via GalNAc LNPs in PHH cells (lot IRZ) by LNP transfection in two separate experiments. Control groups were two different GalNAc LNPs containing gRNA GA441 (2′-OMe-enriched tracrRNA) and MA079. The doses were 5,000 ng/TA/mL for one experiment and 10,000 ng/TA/mL for the other experiment. On-target primer (GAO100_OT_B1) was used to measure editing percentage (A>G) in positions 1-10 of protospacer region in the ANGPTL3 gene by Targeted Amplicon sequencing. Technical replicates were performed (FIG. 36A presents data from this study).

Table 5 shows the LNP formulations used for generating LNP transfection in vitro data described in FIGS. 36A, 36B, 37, 39A, 39B, 40, 41A and 41B.

TABLE 5
LNP formulations

| | | LNPs | | | | |
|---|---|---|---|---|---|---|
| | | Composition: Amino Lipid (502)/ | | | | |
| | | Cholesterol/DSPC/PEG-Lipid (507)/ | | | | |
| | | GalNAc-Lipid (1004) (Reference: | | | | |
| | | U.S. Pat. No. 11,207,416 B2, which | | | | |
| | | is incorporated herein by reference | | | | |
| | | in its entirety) | | | | |
| Total RNA | | | | | PDI | |
| payload | | Formu- | | % | (poly- | |
| 1:1 by wt | | lation | | Encapsu- | dispersity | |
| gRNA | mRNA | ID | Size | lation | index) | |
| GA441 | MA079 | VF1155 | 62.25 | 99.4 | 0.0280 | 2′-OMe- |
| GA441 | MA079 | VF1272 | 66.68 | 99.2 | 0.0073 | enriched |
| GA441 | MA079 | VF1313 | 71.55 | 99.5 | 0.0441 | tracer |
| GA091 | MA079 | VF1321 | 72.43 | 99.5 | 0.0573 | Minimally |
| GA675 | MA079 | VF1273 | 66.51 | 99.2 | 0.0555 | modified |
| GA677 | MA079 | VF1274 | 67.7 | 99.3 | 0.0353 | tracer |
| GA677 | MA079 | VF1312 | 72.06 | 99.5 | 0.0278 | |
| GA678 | MA079 | VF1275 | 66.32 | 99.1 | 0.0347 | |
| GA680 | MA079 | VF1280 | 66.3 | 99.4 | 0.0335 | |
| GA682 | MA079 | VF1276 | 67.67 | 99.2 | 0.0251 | |
| GA682 | MA079 | VF1314 | 71.8 | 99.5 | 0.0534 | |
| GA683 | MA079 | VF1281 | 66.08 | 99.4 | 0.0423 | |
| GA685 | MA079 | VF1282 | 64.63 | 99.5 | 0.0206 | |
| GA686 | MA079 | VF1283 | 64.3 | 99.5 | 0.0183 | |
| GA686 | MA079 | VF1315 | 71.79 | 99.4 | 0.0310 | |
| GA687 | MA079 | VF1286 | 66.52 | 99 | 0.0028 | |
| GA688 | MA079 | VF1284 | 65.15 | 99.5 | 0.0201 | |
| GA689 | MA079 | VF1288 | 65.11 | 99.5 | 0.0433 | |
| GA690 | MA079 | VF1287 | 65.76 | 99.5 | 0.0064 | |
| GA690 | MA079 | VF1316 | 73.57 | 99.4 | 0.0255 | |
| GA691 | MA079 | VF1277 | 68.04 | 99.2 | 0.0385 | |
| GA691 | MA079 | VF1317 | 76.99 | 98.1 | 0.0658 | |
| GA692 | MA079 | VF1278 | 66.03 | 99.4 | 0.0306 | |
| GA692 | MA079 | VF1318 | 76.44 | 99.1 | 0.0330 | |
| GA693 | MA079 | VF1285 | 66.07 | 99.5 | 0.0250 | |
| GA693 | MA079 | VF1319 | 72 | 99.5 | 0.0334 | |
| GA694 | MA079 | VF1279 | 67.42 | 99.2 | 0.0209 | |
| GA694 | MA079 | VF1320 | 71.88 | 99.6 | 0.0496 | |
| GA837 | MA079 | VF1542 | 64.15 | 99.05 | 0.06335 | |

The genomic DNA from PHH cells transfected with LNPs containing one of 16 sgRDNA spacer sequence variants were further tested for off-target editing. (FIG. 36B). Samples dosed with 10,000 ng/TA/mL were assessed, in addition to one of the control groups dosed with 5,000 ng/TA/mL. OT1 primer (GAO100_OT_B2) was used to measure editing percentage (A>G) in positions 1-10 of the OT1 site by Targeted Amplicon sequencing. Technical replicates were performed.

The genomic DNA from PHH cells transfected by LNP with 16 sgRDNA spacer sequence variants were further tested for off-target editing according to the protocol of SureSelect (FIG. 37 presents data from this study). The same genomic DNA samples were used for Targeted Amplicon. The input DNA was 100 ng and a panel of RNA probes of 611 candidate off-target sites derived from ONE-seq with GA441. The plot shows net editing percentage of significant sites (non-adjusted pᵥₐₗ<0.05) and is ordered by on-target potency.

FIGS. 38A & 39A provide subset of SureSelect data of top sgRDNA spacer sequence variants and the corresponding Targeted Amplicon data is in FIGS. 38B & 39B. The PHH cells in FIGS. 38A-38B were transfected by LNP and the PHH cells in FIGS. 39A-39B were transfected by MessengerMax™. These results have consistently shown that the selected spacer sequences can achieve high on-target editing but low off-target editing.

### Example 11. LNP Dose response curves for On-target and Off-target editing in PHH

LNPs were generated either by (1) microfluidic mixing using the Precision Nanosystems NanoAssemblr system according to the manufacturer's protocol, with some optimization for individual payloads or by (2) rapid inline mixing of a solution of lipid excipients in an organic solvent and an aqueous solution of gRNA and mRNA. The lipid solution generally comprises of a mixture of four formulation excipients namely: an amino lipid, a polyethylene glycol of average molecular weight 2000 Da conjugated to a lipid called PEG-Lipid, cholesterol and 1,2-distearoyl-sn-glycero-3-phosphocholine (DSPC) and GalNAc-Lipid 1004 (described in U.S. Ser. No. 11/207,416 B2, which is incorporated herein by reference in its entirety), mixed in a predetermined molar ratio in ethanol. LNP composition comprised 40-65% amino lipid, 2-20% DSPC, 1-5% PEG-Lipid, and 0.01-1% GalNAc-Lipid with the balance being cholesterol (all in mol %). The RNA aqueous solution contains a 1:1 by weight mixture of desired mRNA and guide RNA (gRNA) unless otherwise specified. The aqueous solution of desired mRNA to gRNA was then mixed with the lipid excipients in ethanol by microfluidic or by rapid inline mixing. All LNPs for reported NHP studies were prepared by following the inline mixing protocols as described in WO 2022/060871A 1 and U.S. Ser. No. 11/207,416 B2, which are incorporated herein by reference in their entirety.

Sixteen (16) different gRDNA comprising the spacer sequences with minimally modified tracrRNA were formulated with in different LNP (Table 5) and transfected at different doses to PHH cells (lot IRZ) to assess LNP dose-response. Doses included 10,000 ng/TA/mL, 5,000 ng/TA/mL, 2,500 ng/TA/mL, 1,250 ng/TA/mL, 625 ng/TA/mL, and 312.5 ng/TA/mL.

Two different batches of GalNAc-LNPs containing either 8 gRDNA spacer sequence variants with minimally modified tracrRNA or controls, (a) GA091 with minimally modified tracrRNA and (b) GA441 with 2′-OMe-enriched tracrRNA, and mRNA MA079 were formulated and transfectedin PHH cells (lot IRZ). Doses were 10,000 ng/TA/mL, 5,000 ng/TA/mL, 2,500 ng/TA/mL, 1,250 ng/TA/mL, 625 ng/TA/mL, and 312.5 ng/TA/mL. On-target primer (GA0100_OT_B1) was used to measure editing percentage (A>N) in position 6 of protospacer region in the ANGPTL3 gene by Targeted Amplicon sequencing (FIG. 40). OT1 primer (GA0100_OT_B2) and OT3 primer (GA0100_OT_B6) were used to measure off-target editing percentage (A>G) in positions 1-10 of off-target sites (FIGS. 41A-41B).

A GalNAc-LNP containing guide GA837, which is a gRDNA comprising a spacer sequence and a 2′-OMe-enriched tracrRNA, along with mRNA MA079, was formulated and transfected in PHH cells from four different donors (GNA, IRZ, LFQ, NFX). Doses were 45,000 ng/TA/mL, 30,000 ng/TA/mL, 20,000 ng/TA/mL, 13,333 ng/TA/mL, 8,889 ng/TA/mL, 4,444 ng/TA/mL, 2,222 ng/TA/mL, 1,111 ng/TA/mL, 556 ng/TA/mL, 278 ng/TA/mL, 139 ng/TA/mL and 69 ng/TA/mL. On-target primer (GA0100_OT_B1) was used to measure editing percentage (A>N) in position 6 of protospacer region in the ANGPTL3 gene by Targeted Amplicon sequencing (FIG. 42).

The evaluation of the potential for the gRDNAs to introduce structural variants (SVs) is at the nexus between off-target editing risks and genotoxicity. While karyotyping can be used to evaluate the presence of SVs and clastogenicity, it is a low-throughput, visual assay with low resolution. To confirm the absence of mutagenesis and clastogenicity with X, the Bionano Saphyr optical genome mapping (OGM) method (Bionano Genomics, 2020; Cao et al., 2014; Mak et al., 2016) was used to assess for any evidence of SVs on a genome-wide basis. OGM is a newer, imaging-based approach analogous to karyotyping but performed with greater sensitivity and at a much higher resolution, based on whole-genome coverage >1200×. The sponsor demonstrated approximately 90% detection rate of deletions at 5% VAF and of insertions at 1% VAF, using image data with >1200× effective genome coverage.

Genomic DNA from PHH cells (lot IRZ) transfected with LNP VF1542 at a dose of 20,000 ng/TA/mL was assessed for on-target editing using primer GA0100_OT_B1 (FIG. 43A). This genomic DNA was then analyzed by OGM. Total events across all SV categories (insertions, duplications, deletions, inversions, and translocations) from untreated and treated samples were summed and plotted as Circos plots. The subtraction of SVs from the treated samples that are present in the untreated control sample resulted in no remaining SVs (i.e., no SVs were generated after treatment with VF1542) (FIG. 43B).

### Example 12. On-Target and Off-Target Editing Rates in Primary Cyno Hepatocytes (PCH)

Next, hepatic ANGPTL3 editing by sgRDNA spacer sequences was measured in primary cyno hepatocytes (PCH). The results suggest that hepatic ANGPTL3 editing in LNP treated NHPs correlates with editing in PCH (FIGS. 44-45).

### Example 13. Treatment in Non-Human Primates (NHP)

Two NHP studies were conducted using LNPs constituted with two select set of spacer-modified guide nucleic acids comprising the same spacer sequences and the mRNA MA004 or MA079. The LNPs were formulated as described in Example 2. Table 6 shows the LNP formulations used for dosing in NHP (described in FIGS. 44, 46 and 47) and transfecting PCH cell lines (described in FIG. 45). Three digits after decimal point following formulation, gRNA and mRNA IDs indicate lot or batch number of the respective formulation, gRNA and mRNA.

TABLE 6
LNP Formulations

| | | | LNPs Composition: Amino Lipid (502)/ | | | | |
|---|---|---|---|---|---|---|---|
| | | | Cholesterol/DSPC/PEG-Lipid (507)/GalNAc-Lipid | | | | |
| | Total RNA | | (1004) (Reference: U.S. Pat. No. 11,207,416 B2, which | | | | |
| LNP | payload 1:1 by wt | | is incorporated herein by reference in its entirety) | | | | |
| test | Cyno | | | | % | | |
| articles | gRNA | mRNA | Formulation | Size | Encapsulation | PDI | Study |
| | GA347 | MA079 | VF1355 | 67.92 | 98.4 | 0.08996 | PCH in vitro Transfection |
| LNP 1 | GA347 | MA004 | VF1185 | 64.42 | 98.9 | 0.05933 | PCH in vitro Transfection, NHP Study 1 |
| | GA347 | MA079 | VF1365 | 70.87 | 99.0 | 0.01249 | PCH in vitro Transfection |
| LNP 1 | GA347 | MA079 | VF1371 | 64.76 | 98.9 | 0.05064 | NHP Study 2 |
| | GA748 | MA079 | VF1366 | 82.78 | 97.5 | 0.01597 | PCH in vitro Transfection |
| LNP 7 | GA748 | MA079 | VF1372 | 64.09 | 99.0 | 0.04973 | NHP Study 2 |
| | GA749 | MA079 | VF1367 | 68.27 | 99.2 | 0.02219 | PCH in vitro Transfection |
| LNP 8 | GA749 | MA079 | VF1373 | 66.40 | 99.2 | 0.04976 | NHP study 2 |
| LNP 2 | GA663 | MA004 | VF1215 | 65.40 | 99.1 | 0.0075 | PCH in vitro Transfection, NHP Study 1 |
| | GA663 | MA079 | VF1368 | 73.19 | 99.3 | 0.04705 | PCH in vitro Transfection |
| | GA663 | MA079 | VF1374 | 65.54 | 99.1 | 0.002 | PCH in vitro Transfection |
| LNP 4 | GA668 | MA004 | VF1230 | 61.18 | 99.0 | 0.072 | PCH in vitro Transfection, NHP Study 1 |
| | GA668 | MA079 | VF1369 | 69.01 | 98.9 | 0.006209 | PCH in vitro Transfection |
| LNP 4 | GA668 | MA079 | VF1375 | 63.67 | 99.4 | 0.009886 | NHP study 2 |
| | GA747 | MA079 | VF1370 | 67.61 | 99.2 | 0.0408 | PCH in vitro Transfection |
| | GA747 | MA079 | VF1376 | 68.69 | 99.1 | 0.01766 | PCH in vitro Transfection |
| LNP 3 | GA666 | MA004 | VF1219 | 63.99 | 99.1 | 0.06683 | PCH in vitro Transfection, NHP Study 1 |
| LNP 5 | GA665 | MA004 | VF1237 | 60.11 | 99.2 | 0.02243 | PCH in vitro Transfection, NHP Study 1 |
| LNP 6 | GA720 | MA004 | VF1240 | 61.03 | 99.3 | 0.01343 | PCH in vitro Transfection, NHP Study 1 |

For the first study, female cynomolgus monkeys of Cambodian origin were used as study animals. Three monkeys were dosed with LNP1 (GA347), 3 monkeys were dosed with LNP2 (GA663), 3 monkeys were dosed with LNP3 (GA666), 3 monkeys with LNP4 (GA668), 3 monkeys with LNP5 (GA665) and 3 monkeys with LNP6 (GA720) on day 1 of the study via a single IV infusion at a dose level of 2 mg of combined sgRNA and mRNA per kg of animal body weight and at a dose volume of 6 mL/kg (n=3/group). Blood samples were collected from all animals predose for baseline measurement and post-dose at various time points on days 1 through 15 to assess biomarkers and serum safety parameters. Necropsies were performed on all animals at day 16. Liver biopsy samples were collected to assess ANGPTL3 gene editing.

For second the study, female cynomolgus monkeys of Cambodian origin were used as study animals. Three monkeys were dosed with LNP1 (GA347) at a dose level of 2 mg/kg, 2 groups of monkeys (n=3/group) were dosed with LNP7 (GA748) at a dose level of 2 mg/kg or a dose level of 4 mg/kg, 3 monkeys with LNP4 (GA668), and 3 groups of monkeys with LNP8 (GA749) at a dose level of 2, 3, and 4 mg/kg respectively. All LNPs were administered via a single IV infusion on day 1 of the study at the indicated dose level of combined sgRNA and mRNA per kg of animal body weight and at a dose volume of 6 mL/kg. Blood samples were collected from all animals predose for baseline measurement and post-dose at various time points on days 1 through 15 to assess biomarkers and serum safety parameters. Necropsies were performed on all animals at day 16. Liver biopsy samples were collected to assess ANGPTL3 gene editing.

The extent of gene editing in the liver was evaluated by next-generation sequencing (NGS) of targeted polymerase chain reaction (PCR) amplicons (Targeted Amplicon sequencing) at the ANGPTL3 target site, using species-specific primer cAN_Ex6_3_NEXT_F1, derived from genomic DNA extracted from the liver of the animal using the method described previously (Musunuru et al, Nature 593, no. 7859 (May 2021): 429-34. https://doi.org/10.1038/s41586-021-03534-y). Percent editing was reported as the percent of all reads containing a nonreference allele at the target adenine.

Serum was collected from all animals on days −10, −7, −5 pre-infusion and days 8 and 15 post LNP infusion for ANGPTL3 protein analysis. ANGPTL3 protein levels were quantified using an ANGPTL3 sandwich ELISA with the data obtained from the analysis of Day 15 samples presented in FIG. 47. Values for day −10, −7, and −5 were averaged to obtain the baseline value.

The results in NHP Cynomolgus monkeys show that the guide nucleic acid sequences comprising the spacer sequences disclosed herein were able to achieve >54% editing (A>N) at position 6 in the protospacer region of the ANGPL3 gene by Targeted Amplicon sequencing in liver 15 days post injection (FIG. 46). There was >90% reduction in plasma ANGPTL3 protein compared to baseline, 15 days post injection. (FIG. 47).

The spacer sequences and the guide nucleic acid sequences comprising the spacer sequences targeting human ANGPTL3 gene as well as the corresponding sequences targeting cyno ANGPTL3 gene are provided in Table 7.

TABLE 7
Guide RNA (gRDNA) and Spacer Sequence Pairs Targeting ANGPTL3 gene

| | | | SEQ | | SEQ |
|---|---|---|---|---|---|
| | gRDNA | Spacer | ID | | ID |
| Species | ID | Sequence | NO | gRDNA Sequence | NO |
| Human | GA091 | asasg | 5 | asasgsAUACCUGAAUAACCCUCGUUUUAGAG | 69 |
| (1) | | sAUAC | | CUAGAAAUAGCAAGUUAAAAUAAGGCUAG | |
| | | CUGA | | UCCGUUAUCAACUUGAAAAAGUGGCACCG | |
| | | AUAA | | AGUCGGUGCUsususu | |
| | | CCCU | | | |
| | | C | | | |
| | GA441 | | | asasgsAUACCUGAAUAACCCUCgUUUUAGagcu | 70 |
| | | | | aGaaauagcaaGUUaAaAuAaggcuaGUccGUUAucA | |
| | | | | AcuuGaaaaagugGcaccgagucggugcusususu | |
| Monkey | N/A | asasg | 6 | N/A | |
| (1) | | sAUAC | | | |
| | | CUGA | | | |
| | | AUAA | | | |
| | | CUCU | | | |
| | | C | | | |
| | GA347 | | | asasgsAUACCUGAAUAACUCUCgUUUUAGagc | 71 |
| | | | | uaGaaauagcaaGUUaAaAuAaggcuaGUccGUUAucA | |
| | | | | AcuuGaaaaagugGcaccgagucggugcusususu | |
| Human | GA680 | asasg | 7 | asasgsAUAdCCdUGAAdUAACCCUCGUUUUAG | 72 |
| (2) | | sAUAd | | AGCUAGAAAUAGCAAGUUAAAAUAAGGCU | |
| | | CCdU | | AGUCCGUUAUCAACUUGAAAAAGUGGCAC | |
| | | GAAd | | CGAGUCGGUGCUsususu | |
| | | UAAC | | | |
| | | CCUC | | | |
| Monkey | GA765 | asasgs | 8 | asasgsAUAdCCdUGAAdUAACUCUCGUUUUAG | 73 |
| (2) | | AUAd | | AGCUAGAAAUAGCAAGUUAAAAUAAGGCU | |
| | | CCdU | | AGUCCGUUAUCAACUUGAAAAAGUGGCAC | |
| | | GAAd | | CGAGUCGGUGCUsususu | |
| | | UAAC | | | |
| | | UCUC | | | |
| | GA668 | | | asasgsAUAdCCdUGAAdUAACUCUCgUUUUAG | 74 |
| | | | | agcuaGaaauagcaaGUUaAaAuAaggcuaGUccGUUA | |
| | | | | ucAAcuuGaaaaagugGcaccgagucggugcusususu | |
| Human | GA675 | asdAsg | 9 | asdAsgsadUdAdCdCdUdGdAAUdAAdCdCdCUCG | 75 |
| (3) | | sadUd | | UUUUAGAGCUAGAAAUAGCAAGUUAAAAU | |
| | | AdCdC | | AAGGCUAGUCCGUUAUCAACUUGAAAAAG | |
| | | dUdGd | | UGGCACCGAGUCGGUGCUsususu | |
| | | AAUd | | | |
| | | AAdCd | | | |
| | | CdCUC | | | |
| | GA722 | | | asdAsgsadUdAdCdCdUdGdAAUdAAdCdCdCUCg | 76 |
| | | | | UUUUAGagcuaGaaauagcaaGUUaAaAuAaggcuaG | |
| | | | | UccGUUAucAAcuuGaaaaagugGcaccgagucggugcus | |
| | | | | ususu | |
| Monkey | GA762 | asdAsg | 10 | asdAsgsadUdAdCdCdUdGdAAUdAAdCdUdCUC | 77 |
| (3) | | sadUd | | GUUUUAGAGCUAGAAAUAGCAAGUUAAAA | |
| | | AdCdC | | UAAGGCUAGUCCGUUAUCAACUUGAAAAA | |
| | | dUdGd | | GUGGCACCGAGUCGGUGCUsususu | |
| | | AAUd | | | |
| | | AAdCd | | | |
| | | UdCU | | | |
| | | C | | | |
| | GA663 | | | asdAsgsadUdAdCdCdUdGdAAUdAAdCdUdCUCg | 78 |
| | | | | UUUUAGagcuaGaaauagcaaGUUaAaAuAaggcuaG | |
| | | | | UccGUUAucAAcuuGaaaaagugGcaccgagucggugcus | |
| | | | | ususu | |
| Human | GA682 | asasdG | 11 | asasdGsdAdUdAdCCUGAAUAACCCUCGUUUU | 79 |
| (4) | | sdAdU | | AGAGCUAGAAAUAGCAAGUUAAAAUAAGG | |
| | | dAdCC | | CUAGUCCGUUAUCAACUUGAAAAAGUGGC | |
| | | UGAA | | ACCGAGUCGGUGCUsususu | |
| | | UAAC | | | |
| | | CCUC | | | |
| | GA723 | | | asasdGsdAdUdAdCCUGAAUAACCCUCgUUUU | 80 |
| | | | | AGagcuaGaaauagcaaGUUaAaAuAaggcuaGUccGU | |
| | | | | UAucAAcuuGaaaaagugGcaccgagucggugcusususu | |
| Monkey | GA755 | asasdG | 12 | asasdGsdAdUdAdCCUGAAUAACUCUCGUUUU | 81 |
| (4) | | sdAdU | | AGAGCUAGAAAUAGCAAGUUAAAAUAAGG | |
| | | dAdCC | | CUAGUCCGUUAUCAACUUGAAAAAGUGGC | |
| | | UGAA | | ACCGAGUCGGUGCUsususu | |
| | | UAAC | | | |
| | | UCUC | | | |
| | GA746 | | | asasdGsdAdUdAdCCUGAAUAACUCUCgUUUU | 82 |
| | | | | AGagcuaGaaauagcaaGUUaAaAuAaggcuaGUccGU | |
| | | | | UAucAAcuuGaaaaagugGcaccgagucggugcusususu | |
| Human | GA683 | dAsdAs | 13 | dAsdAsgsadUdAdCdCdUdGdAAUdAAdCdCdCU | 83 |
| (5) | | gsadUd | | CGUUUUAGAGCUAGAAAUAGCAAGUUAAA | |
| | | AdCdC | | AUAAGGCUAGUCCGUUAUCAACUUGAAAA | |
| | | dUdGd | | AGUGGCACCGAGUCGGUGCUsususu | |
| | | AAUd | | | |
| | | AAdCd | | | |
| | | CdCUC | | | |
| | GA635 | | | dAsdAsgsadUdAdCdCdUdGdAAUdAAdCdCdCU | 84 |
| | | | | CgUUUUAGagcuaGaaauagcaaGUUaAaAuAaggcua | |
| | | | | GUccGUUAucAAcuuGaaaaagugGcaccgagucggugc | |
| | | | | usususu | |
| Monkey | GA766 | dAsdAs | 14 | dAsdAsgsadUdAdCdCdUdGdAAUdAAdCdUdCU | 85 |
| (5) | | gsadUd | | CGUUUUAGAGCUAGAAAUAGCAAGUUAAA | |
| | | AdCdC | | AUAAGGCUAGUCCGUUAUCAACUUGAAAA | |
| | | dUdGd | | AGUGGCACCGAGUCGGUGCUsususu | |
| | | AAUd | | | |
| | | AAdCd | | | |
| | | UdC | | | |
| Human | GA678 | asdAsd | 15 | asdAsdGsdAdTdAdCdCdTdGAAUAACCCUCGU | 86 |
| (6) | | GsdAd | | UUUAGAGCUAGAAAUAGCAAGUUAAAAUA | |
| | | UdAdC | | AGGCUAGUCCGUUAUCAACUUGAAAAAGU | |
| | | dCdUd | | GGCACCGAGUCGGUGCUsususu | |
| | | GAAU | | | |
| | | AACC | | | |
| | | CUC | | | |
| Monkey | GA764 | asdAsd | 16 | asdAsdGsdAdTdAdCdCdTdGAAUAACUCUCGU | 87 |
| (6) | | GsdAd | | UUUAGAGCUAGAAAUAGCAAGUUAAAAUA | |
| | | UdAdC | | AGGCUAGUCCGUUAUCAACUUGAAAAAGU | |
| | | dCdUd | | GGCACCGAGUCGGUGCUsususu | |
| | | GAAU | | | |
| | | AACU | | | |
| | | CUC | | | |
| | GA666 | | | asdAsdGsdAdTdAdCdCdTdGAAUAACUCUCgU | 88 |
| | | | | UUUAGagcuaGaaauagcaaGUUaAaAuAaggcuaGUc | |
| | | | | cGUUAucAAcuuGaaaaagugGcaccgagucggugcususu | |
| | | | | su | |
| Human | GA677 | asasdG | 17 | asasdGsdAdUdACdCUdGAdAUdAAdCCdCUdCG | 89 |
| (7) | | sdAdU | | UUUUAGAGCUAGAAAUAGCAAGUUAAAAU | |
| | | dACdC | | AAGGCUAGUCCGUUAUCAACUUGAAAAAG | |
| | | UdGAd | | UGGCACCGAGUCGGUGCUsususu | |
| | | AUdA | | | |
| | | AdCCd | | | |
| | | CUdC | | | |
| | GA629 | | | asasdGsdAdUdACdCUdGAdAUdAAdCCdCUdCg | 90 |
| | | | | UUUUAGagcuaGaaauagcaaGUUaAaAuAaggcuaG | |
| | | | | UccGUUAucAAcuuGaaaaagugGcaccgagucggugcus | |
| | | | | ususu | |
| Monkey | GA763 | asasdG | 18 | asasdGsdAdUdACdCUdGAdAUdAAdCUdCUdCG | 91 |
| (7) | | sdAdU | | UUUUAGAGCUAGAAAUAGCAAGUUAAAAU | |
| | | dACdC | | AAGGCUAGUCCGUUAUCAACUUGAAAAAG | |
| | | UdGAd | | UGGCACCGAGUCGGUGCUsususu | |
| | | AUdA | | | |
| | | AdCUd | | | |
| | | CUdC | | | |
| | GA665 | | | asasdGsdAdUdACdCUdGAdAUdAAdCUdCUdCg | 92 |
| | | | | UUUUAGagcuaGaaauagcaaGUUaAaAuAaggcuaG | |
| | | | | UccGUUAucAAcuuGaaaaagugGcaccgagucggugcus | |
| | | | | ususu | |
| Human | GA676 | asasdG | 19 | asasdGsdAdUdAdCdCdUdGAdAUdAAdCCdCUdC | 93 |
| (8) | | sdAdU | | GUUUUAGAGCUAGAAAUAGCAAGUUAAAA | |
| | | dAdCd | | UAAGGCUAGUCCGUUAUCAACUUGAAAAA | |
| | | CdUdG | | GUGGCACCGAGUCGGUGCUsususu | |
| | | AdAUd | | | |
| | | AAdC | | | |
| | | CdCUd | | | |
| | | C | | | |
| | GA630 | | | asasdGsdAdUdAdCdCdUdGAdAUdAAdCCdCUdC | 94 |
| | | | | gUUUUAGagcuaGaaauagcaaGUUaAaAuAaggcua | |
| | | | | GUccGUUAucAAcuuGaaaaagugGcaccgagucggugc | |
| | | | | usususu | |
| Monkey | GA664 | asasdG | 20 | asasdGsdAdUdAdCdCdUdGAdAUdAAdCUdCUd | 95 |
| (8) | | sdAdU | | CgUUUUAGagcuaGaaauagcaaGUUaAaAuAaggcua | |
| | | dAdCd | | GUccGUUAucAAcuuGaaaaagugGcaccgagucggugc | |
| | | CdUdG | | usususu | |
| | | AdAUd | | | |
| | | AAdC | | | |
| | | UdCUd | | | |
| | | C | | | |
| Human | GA679 | dAsdAs | 21 | dAsdAsdGsdAdUdACdCdUdGdAAUAACCCUCG | 96 |
| (9) | | dGsdA | | UUUUAGAGCUAGAAAUAGCAAGUUAAAAU | |
| | | dUdAC | | AAGGCUAGUCCGUUAUCAACUUGAAAAAG | |
| | | dCdUd | | UGGCACCGAGUCGGUGCUsususu | |
| | | GdAA | | | |
| | | UAAC | | | |
| | | CCUC | | | |
| Monkey | GA667 | dAsdAs | 22 | dAsdAsdGsdAdUdACdCdUdGdAAUAACUCUCg | 97 |
| (9) | | dGsdA | | UUUUAGagcuaGaaauagcaaGUUaAaAuAaggcuaG | |
| | | dUdAC | | UccGUUAucAAcuuGaaaaagugGcaccgagucggugcus | |
| | | dCdUd | | ususu | |
| | | GdAA | | | |
| | | UAAC | | | |
| | | UCUC | | | |
| Human | GA681 | asdAsd | 23 | asdAsdGsdAdUdAdCdCdUdGAdAUdAAdCCdCU | 98 |
| (10) | | GsdAd | | dCGUUUUAGAGCUAGAAAUAGCAAGUUAA | |
| | | UdAdC | | AAUAAGGCUAGUCCGUUAUCAACUUGAAA | |
| | | dCdUd | | AAGUGGCACCGAGUCGGUGCUsususu | |
| | | GAdA | | | |
| | | UdAAd | | | |
| | | CcdCU | | | |
| | | dC | | | |
| | GA669 | asdAsd | 24 | asdAsdGsdAdUdAdCdCdUdGAdAUdAAdCUdCU | 99 |
| | | GsdAd | | dCgUUUUAGagcuaGaaauagcaaGUUaAaAuAaggc | |
| | | UdAdC | | uaGUccGUUAucAAcuuGaaaaagugGcaccgagucggu | |
| | | dCdUd | | gcusususu | |
| | | GAdA | | | |
| | | UdAAd | | | |
| | | CUdC | | | |
| | | UdC | | | |
| Human | GA684 | asasdG | 25 | asasdGsdAdUdAdCdCUGAAUAACCCUCGUUU | 100 |
| (11) | | sdAdU | | UAGAGCUAGAAAUAGCAAGUUAAAAUAAG | |
| | | dAdCd | | GCUAGUCCGUUAUCAACUUGAAAAAGUGG | |
| | | CUGA | | CACCGAGUCGGUGCUsususu | |
| | | AUAA | | | |
| | | CCCU | | | |
| | | C | | | |
| | GA636 | | | asasdGsdAdUdAdCdCUGAAUAACCCUCgUUUU | 101 |
| | | | | AGagcuaGaaauagcaaGUUaAaAuAaggcuaGUccGU | |
| | | | | UAucAAcuuGaaaaagugGcaccgagucggugcusususu | |
| Human | GA691 | asasgsd | 26 | asasgsdAdUdAdCCdUdGAAdUdAACCCUCGUU | 102 |
| (12) | | AdUdA | | UUAGAGCUAGAAAUAGCAAGUUAAAAUAA | |
| | | dCCdU | | GGCUAGUCCGUUAUCAACUUGAAAAAGUG | |
| | | dGAAd | | GCACCGAGUCGGUGCUsususu | |
| | | UdAA | | | |
| | | CCCU | | | |
| | | C | | | |
| | GA724 | | | asasgsdAdUdAdCCdUdGAAdUdAACCCUCgUUU | 103 |
| | | | | UAGagcuaGaaauagcaaGUUaAaAuAaggcuaGUccG | |
| | | | | UUAucAAcuuGaaaaagugGcaccgagucggugcusususu | |
| Monkey | GA757 | asasgsd | 27 | asasgsdAdUdAdCCdUdGAAdUdAACUCUCGUU | 104 |
| (12) | | AdUdA | | UUAGAGCUAGAAAUAGCAAGUUAAAAUAA | |
| | | dCCdU | | GGCUAGUCCGUUAUCAACUUGAAAAAGUG | |
| | | dGAAd | | GCACCGAGUCGGUGCUsususu | |
| | | UdAA | | | |
| | | CUCU | | | |
| | | C | | | |
| | GA747 | | | asasgsdAdUdAdCCdUdGAAdUdAACUCUCgUUU | 105 |
| | | | | UAGagcuaGaaauagcaaGUUaAaAuAaggcuaGUccG | |
| | | | | UUAucAAcuuGaaaaagugGcaccgagucggugcusususu | |
| Human | GA692 | asasdG | 28 | asasdGsdAUdAdCCUGAAUAACCCUCGUUUUA | 106 |
| (13) | | sdAUd | | GAGCUAGAAAUAGCAAGUUAAAAUAAGGC | |
| | | AdCC | | UAGUCCGUUAUCAACUUGAAAAAGUGGCA | |
| | | UGAA | | CCGAGUCGGUGCUsususu | |
| | | UAAC | | | |
| | | CCUC | | | |
| | GA745 | | | asasdGsdAUdAdCCUGAAUAACCCUCgUUUUA | 107 |
| | | | | GagcuaGaaauagcaaGUUaAaAuAaggcuaGUccGUU | |
| | | | | AucAAcuuGaaaaagugGcaccgagucggugcusususu | |
| Monkey | GA758 | asasdG | 29 | asasdGsdAUdAdCCUGAAUAACUCUCGUUUUA | 108 |
| (13) | | sdAUd | | GAGCUAGAAAUAGCAAGUUAAAAUAAGGC | |
| | | AdCC | | UAGUCCGUUAUCAACUUGAAAAAGUGGCA | |
| | | UGAA | | CCGAGUCGGUGCUsususu | |
| | | UAAC | | | |
| | | UCUC | | | |
| | GA748 | | | asasdGsdAUdAdCCUGAAUAACUCUCgUUUUA | 109 |
| | | | | GagcuaGaaauagcaaGUUaAaAuAaggcuaGUccGUU | |
| | | | | AucAAcuuGaaaaagugGcaccgagucggugcusususu | |
| Human | GA693 | asasdG | 30 | asasdGsdAUdAdCdCUGAAUAACCCUCGUUUU | 110 |
| (14) | | sdAUd | | AGAGCUAGAAAUAGCAAGUUAAAAUAAGG | |
| | | AdCdC | | CUAGUCCGUUAUCAACUUGAAAAAGUGGC | |
| | | UGAA | | ACCGAGUCGGUGCUsususu | |
| | | UAAC | | | |
| | | CCUC | | | |
| | GA837 | | | asasdGsdAUdAdCdCUGAAUAACCCUCgUUUU | 111 |
| | | | | AGagcuaGaaauagcaaGUUaAaAuAaggcuaGUccGU | |
| | | | | UAucAAcuuGaaaaagugGcaccgagucggugcusususu | |
| Monkey | GA759 | asasdG | 31 | asasdGsdAUdAdCdCUGAAUAACUCUCGUUUU | 112 |
| (14) | | sdAUd | | AGAGCUAGAAAUAGCAAGUUAAAAUAAGG | |
| | | AdCdC | | CUAGUCCGUUAUCAACUUGAAAAAGUGGC | |
| | | UGAA | | ACCGAGUCGGUGCUsususu | |
| | | UAAC | | | |
| | | UCUC | | | |
| | GA749 | | | asasdGsdAUdAdCdCUGAAUAACUCUCgUUUU | 113 |
| | | | | AGagcuaGaaauagcaaGUUaAaAuAaggcuaGUccGU | |
| | | | | UAucAAcuuGaaaaagugGcaccgagucggugcusususu | |
| Human | GA690 | asasgs | 32 | asasgsAUACCUdGAAdUAACCdCUCGUUUUAG | 114 |
| (15) | | AUAC | | AGCUAGAAAUAGCAAGUUAAAAUAAGGCU | |
| | | CUdG | | AGUCCGUUAUCAACUUGAAAAAGUGGCAC | |
| | | AAdU | | CGAGUCGGUGCUsususu | |
| | | AACC | | | |
| | | dCUC | | | |
| | GA642 | | | asasgsAUACCUdGAAdUAACCdCUCgUUUUAG | 115 |
| | | | | agcuaGaaauagcaaGUUaAaAuAaggcuaGUccGUUA | |
| | | | | ucAAcuuGaaaaagugGcaccgagucggugcusususu | |
| Human | GA689 | asasgs | 33 | asasgsAUdACCUGAAUAAdCCCUCGUUUUAG | 116 |
| (16) | | AUdA | | AGCUAGAAAUAGCAAGUUAAAAUAAGGCU | |
| | | CCUG | | AGUCCGUUAUCAACUUGAAAAAGUGGCAC | |
| | | AAUA | | CGAGUCGGUGCUsususu | |
| | | AdCCC | | | |
| | | UC | | | |
| | GA641 | | | asasgsAUdACCUGAAUAAdCCCUCgUUUUAGa | 117 |
| | | | | gcuaGaaauagcaaGUUaAaAuAaggcuaGUccGUUAu | |
| | | | | cAAcuuGaaaaagugGcaccgagucggugcusususu | |
| Monkey | GA770 | asasgs | 34 | asasgsAUdACCUGAAUAAdCUCUCGUUUUAG | 118 |
| (16) | | AUdA | | AGCUAGAAAUAGCAAGUUAAAAUAAGGCU | |
| | | CCUG | | AGUCCGUUAUCAACUUGAAAAAGUGGCAC | |
| | | AAUA | | CGAGUCGGUGCUsususu | |
| | | AdCU | | | |
| | | CUC | | | |
| | GA0746 | | | asasgsAUdACCUGAAUAAdCUCUCgUUUUAGa | 119 |
| | | | | gcuaGaaauagcaaGUUaAaAuAaggcuaGUccGUUAu | |
| | | | | cAAcuuGaaaaagugGcaccgagucggugcusususu | |
| Human | GA685 | asasgs | 35 | asasgs AdUAdCCdUGdAAdUAdACdCCdUCGUU | 120 |
| (17) | | AdUAd | | UUAGAGCUAGAAAUAGCAAGUUAAAAUAA | |
| | | CCdU | | GGCUAGUCCGUUAUCAACUUGAAAAAGUG | |
| | | GdAAd | | GCACCGAGUCGGUGCUsususu | |
| | | UAdA | | | |
| | | CdCCd | | | |
| | | UC | | | |
| | GA627 | | | asasgsAdUAdCCdUGdAAdUAdACdCCdUCgUUU | 121 |
| | | | | UAGagcuaGaaauagcaaGUUaAaAuAaggcuaGUccG | |
| | | | | UUAucAAcuuGaaaaagugGcaccgagucggugcusususu | |
| Monkey | GA767 | asasgs | 36 | asasgsAdUAdCCdUGdAAdUAdACdUCdUCGUU | 122 |
| (17) | | AdUAd | | UUAGAGCUAGAAAUAGCAAGUUAAAAUAA | |
| | | CCdU | | GGCUAGUCCGUUAUCAACUUGAAAAAGUG | |
| | | GdAAd | | GCACCGAGUCGGUGCUsususu | |
| | | UAdA | | | |
| | | CdUCd | | | |
| | | UC | | | |
| | GA0709 | | | asasgsAdUAdCCdUGdAAdUAdACdUCdUCgUUU | 123 |
| | | | | UAGagcuaGaaauagcaaGUUaAaAuAaggcuaGUccG | |
| | | | | UUAucAAcuuGaaaaagugGcaccgagucggugcusususu | |
| Human | GA687 | asasgsd | 37 | asasgsdAdUdAdCdCdUdGAAUAACCCUCGUUU | 124 |
| (18) | | AdUdA | | UAGAGCUAGAAAUAGCAAGUUAAAAUAAG | |
| | | dCdCd | | GCUAGUCCGUUAUCAACUUGAAAAAGUGG | |
| | | UdGA | | CACCGAGUCGGUGCUsususu | |
| | | AUAA | | | |
| | | CCCU | | | |
| | | C | | | |
| | GA639 | | | asasgsdAdUdAdCdCdUdGAAUAACCCUCgUUU | 125 |
| | | | | UAGagcuaGaaauagcaaGUUaAaAuAaggcuaGUccG | |
| | | | | UUAucAAcuuGaaaaagugGcaccgagucggugcusususu | |
| Monkey | GA768 | asasgsd | 38 | asasgsdAdUdAdCdCdUdGAAUAACUCUCGUUU | 126 |
| (18) | | AdUdA | | UAGAGCUAGAAAUAGCAAGUUAAAAUAAG | |
| | | dCdCd | | GCUAGUCCGUUAUCAACUUGAAAAAGUGG | |
| | | UdGA | | CACCGAGUCGGUGCUsususu | |
| | | AUAA | | | |
| | | CUCU | | | |
| | | C | | | |
| | GA0664 | | | asasgsdAdUdAdCdCdUdGAAUAACUCUCgUUU | 127 |
| | | | | UAGagcuaGaaauagcaaGUUaAaAuAaggcuaGUccG | |
| | | | | UUAucAAcuuGaaaaagugGcaccgagucggugcusususu | |
| Human | GA686 | asasdG | 39 | asasdGsdAdUdAdCdCdUGAAUAACCCUCGUUU | 128 |
| (19) | | sdAdU | | UAGAGCUAGAAAUAGCAAGUUAAAAUAAG | |
| | | dAdCd | | GCUAGUCCGUUAUCAACUUGAAAAAGUGG | |
| | | CdUG | | CACCGAGUCGGUGCUsususu | |
| | | AAUA | | | |
| | | ACCC | | | |
| | | UC | | | |
| | GA638 | | | asasdGsdAdUdAdCdCdUGAAUAACCCUCgUUU | 129 |
| | | | | UAGagcuaGaaauagcaaGUUaAaAuAaggcuaGUccG | |
| | | | | UUAucAAcuuGaaaaagugGcaccgagucggugcusususu | |
| Monkey | GA760 | asasdG | 40 | asasdGsdAdUdAdCdCdUGAAUAACUCUCGUUU | 130 |
| (19) | | sdAdU | | UAGAGCUAGAAAUAGCAAGUUAAAAUAAG | |
| | | dAdCd | | GCUAGUCCGUUAUCAACUUGAAAAAGUGG | |
| | | CdUG | | CACCGAGUCGGUGCUsususu | |
| | | AAUA | | | |
| | | ACUC | | | |
| | | UC | | | |
| | GA750 | | | asasdGsdAdUdAdCdCdUGAAUAACUCUCgUUU | 131 |
| | | | | UAGagcuaGaaauagcaaGUUaAaAuAaggcuaGUccG | |
| | | | | UUAucAAcuuGaaaaagugGcaccgagucggugcusususu | |
| Human | GA694 | asasdG | 41 | asasdGsdAUdAdCdCdUGAAUAACCCUCGUUU | 132 |
| (20) | | sdAUd | | UAGAGCUAGAAAUAGCAAGUUAAAAUAAG | |
| | | AdCdC | | GCUAGUCCGUUAUCAACUUGAAAAAGUGG | |
| | | dUGA | | CACCGAGUCGGUGCUsususu | |
| | | AUAA | | | |
| | | CCCU | | | |
| | | C | | | |
| | GA646 | | | asasdGsdAUdAdCdCdUGAAUAACCCUCgUUUU | 133 |
| | | | | AGagcuaGaaauagcaaGUUaAaAuAaggcuaGUccGU | |
| | | | | UAucAAcuuGaaaaagugGcaccgagucggugcusususu | |
| Monkey | GA761 | asasdG | 42 | asasdGsdAUdAdCdCdUGAAUAACUCUCGUUU | 134 |
| (20) | | sdAUd | | UAGAGCUAGAAAUAGCAAGUUAAAAUAAG | |
| | | AdCdC | | GCUAGUCCGUUAUCAACUUGAAAAAGUGG | |
| | | dUGA | | CACCGAGUCGGUGCUsususu | |
| | | AUAA | | | |
| | | CUCU | | | |
| | | C | | | |
| | GA751 | | | asasdGsdAUdAdCdCdUGAAUAACUCUCgUUUU | 135 |
| | | | | AGagcuaGaaauagcaaGUUaAaAuAaggcuaGUccGU | |
| | | | | UAucAAcuuGaaaaagugGcaccgagucggugcusususu | |
| Human | GA688 | asasdG | 43 | asasdGsdAdUdAdCdCdUdGAAUAACCCUCGUU | 136 |
| (21) | | sdAdU | | UUAGAGCUAGAAAUAGCAAGUUAAAAUAA | |
| | | dAdCd | | GGCUAGUCCGUUAUCAACUUGAAAAAGUG | |
| | | CdUdG | | GCACCGAGUCGGUGCUsususu | |
| | | AAUA | | | |
| | | ACCC | | | |
| | | UC | | | |
| | GA640 | | | asasdGsdAdUdAdCdCdUdGAAUAACCCUCgUU | 137 |
| | | | | UUAGagcuaGaaauagcaaGUUaAaAuAaggcuaGUcc | |
| | | | | GUUAucAAcuuGaaaaagugGcaccgagucggugcususus | |
| | | | | u | |
| Monkey | GA769 | asasdG | 44 | asasdGsdAdUdAdCdCdUdGAAUAACUCUCGUU | 138 |
| (21) | | sdAdU | | UUAGAGCUAGAAAUAGCAAGUUAAAAUAA | |
| | | dAdCd | | GGCUAGUCCGUUAUCAACUUGAAAAAGUG | |
| | | CdUdG | | GCACCGAGUCGGUGCUsususu | |
| | | AAUA | | | |
| | | ACUC | | | |
| | | UC | | | |
| | GA0665 | | | asasdGsdAdUdAdCdCdUdGAAUAACUCUCgUU | 139 |
| | | | | UUAGagcuaGaaauagcaaGUUaAaAuAaggcuaGUcc | |
| | | | | GUUAucAAcuuGaaaaagugGcaccgagucggugcususus | |
| | | | | u | |
| Human | GA695 | asasdG | 45 | asasdGsdAUdAdCdCdUdGAAUAACCCUCGUUU | 140 |
| (22) | | sdAUd | | UAGAGCUAGAAAUAGCAAGUUAAAAUAAG | |
| | | AdCdC | | GCUAGUCCGUUAUCAACUUGAAAAAGUGG | |
| | | dUdGA | | CACCGAGUCGGUGCUsususu | |
| | | AUAA | | | |
| | | CCCU | | | |
| | | C | | | |
| | GA647 | | | asasdGsdAUdAdCdCdUdGAAUAACCCUCgUUU | 141 |
| | | | | UAGagcuaGaaauagcaaGUUaAaAuAaggcuaGUccG | |
| | | | | UUAucAAcuuGaaaaagugGcaccgagucggugcusususu | |
| Human | GA696 | asasdG | 46 | asasdGsdAUAdCdCdUGAAUAACCCUCGUUUU | 142 |
| (23) | | sdAUA | | AGAGCUAGAAAUAGCAAGUUAAAAUAAGG | |
| | | dCdCd | | CUAGUCCGUUAUCAACUUGAAAAAGUGGC | |
| | | UGAA | | ACCGAGUCGGUGCUsususu | |
| | | UAAC | | | |
| | | CCUC | | | |
| | GA648 | | | asasdGsdAUAdCdCdUGAAUAACCCUCgUUUU | 143 |
| | | | | AGagcuaGaaauagcaaGUUaAaAuAaggcuaGUccGU | |
| | | | | UAucAAcuuGaaaaagugGcaccgagucggugcusususu | |
| Human | GA697 | asasdG | 47 | asasdGsdAUAdCdCdUdGAAUAACCCUCGUUU | 144 |
| (24) | | sdAUA | | UAGAGCUAGAAAUAGCAAGUUAAAAUAAG | |
| | | dCdCd | | GCUAGUCCGUUAUCAACUUGAAAAAGUGG | |
| | | UdGA | | CACCGAGUCGGUGCUsususu | |
| | | AUAA | | | |
| | | CCCU | | | |
| | | C | | | |
| | GA649 | | | asasdGsdAUAdCdCdUdGAAUAACCCUCgUUUU | 145 |
| | | | | AGagcuaGaaauagcaaGUUaAaAuAaggcuaGUccGU | |
| | | | | UAucAAcuuGaaaaagugGcaccgagucggugcusususu | |
| Human | GA698 | asasdG | 48 | asasdGsAdUAdCCdUGAAUAACCCUCGUUUUA | 146 |
| (25) | | sAdUA | | GAGCUAGAAAUAGCAAGUUAAAAUAAGGC | |
| | | dCCdU | | UAGUCCGUUAUCAACUUGAAAAAGUGGCA | |
| | | GAAU | | CCGAGUCGGUGCUsususu | |
| | | AACC | | | |
| | | CUC | | | |
| | GA650 | | | asasdGsAdUAdCCdUGAAUAACCCUCgUUUUA | 147 |
| | | | | GagcuaGaaauagcaaGUUaAaAuAaggcuaGUccGUU | |
| | | | | AucAAcuuGaaaaagugGcaccgagucggugcusususu | |
| Human | GA699 | asasgsd | 49 | asasgsdAUdACdCUdGAAUAACCCUCGUUUUA | 148 |
| (26) | | AUdA | | GAGCUAGAAAUAGCAAGUUAAAAUAAGGC | |
| | | CdCUd | | UAGUCCGUUAUCAACUUGAAAAAGUGGCA | |
| | | GAAU | | CCGAGUCGGUGCUsususu | |
| | | AACC | | | |
| | | CUC | | | |
| | GA651 | | | asasgsdAUdACdCUdGAAUAACCCUCgUUUUA | 149 |
| | | | | GagcuaGaaauagcaaGUUaAaAuAaggcuaGUccGUU | |
| | | | | AucAAcuuGaaaaagugGcaccgagucggugcusususu | |
| Human | GA700 | asasgs | 50 | asasgsAUACCdUGAAdUAACCCUCGUUUUAG | 150 |
| (27) | | AUAC | | AGCUAGAAAUAGCAAGUUAAAAUAAGGCU | |
| | | CdUG | | AGUCCGUUAUCAACUUGAAAAAGUGGCAC | |
| | | AAdU | | CGAGUCGGUGCUsususu | |
| | | AACC | | | |
| | | CUC | | | |
| | GA652 | | | asasgsAUACCdUGAAdUAACCCUCgUUUUAGa | 151 |
| | | | | gcuaGaaauagcaaGUUaAaAuAaggcuaGUccGUUAu | |
| | | | | cAAcuuGaaaaagugGcaccgagucggugcusususu | |
| Human | GA701 | asasgsd | 51 | asasgsdAUdACdCUdGAdAUdAAdCCdCUdCGUU | 152 |
| (28) | | AUdA | | UUAGAGCUAGAAAUAGCAAGUUAAAAUAA | |
| | | CdCUd | | GGCUAGUCCGUUAUCAACUUGAAAAAGUG | |
| | | GAdA | | GCACCGAGUCGGUGCUsususu | |
| | | UdAAd | | | |
| | | CCdCU | | | |
| | | dC | | | |
| | GA628 | | | asasgsdAUdACdCUdGAdAUdAAdCCdCUdCgUU | 153 |
| | | | | UUAGagcuaGaaauagcaaGUUaAaAuAaggcuaGUcc | |
| | | | | GUUAucAAcuuGaaaaagugGcaccgagucggugcususus | |
| | | | | u | |
| Monkey | GA0710 | asasgsd | 52 | asasgsdAUdACdCUdGAdAUdAAdCUdCUdCgUU | 154 |
| (28) | | AUdA | | UUAGagcuaGaaauagcaaGUUaAaAuAaggcuaGUcc | |
| | | CdCUd | | GUUAucAAcuuGaaaaagugGcaccgagucggugcususus | |
| | | GAdA | | u | |
| | | UdAAd | | | |
| | | CUdC | | | |
| | | UdC | | | |
| Human | GA702 | asasgs | 53 | asasgsAUACCUGAdAUdAACCCUCGUUUUAG | 155 |
| (28) | | AUAC | | AGCUAGAAAUAGCAAGUUAAAAUAAGGCU | |
| | | CUGA | | AGUCCGUUAUCAACUUGAAAAAGUGGCAC | |
| | | dAUdA | | CGAGUCGGUGCUsususu | |
| | | ACCC | | | |
| | | UC | | | |
| | GA654 | | | asasgsAUACCUGAdAUdAACCCUCgUUUUAGa | 156 |
| | | | | gcuaGaaauagcaaGUUaAaAuAaggcuaGUccGUUAu | |
| | | | | cAAcuuGaaaaagugGcaccgagucggugcusususu | |
| Human | GA703 | sasdGs | 54 | asasdGsAUAdCCUGAdAUdAACCCUCGUUUUA | 157 |
| (29) | | AUAd | | GAGCUAGAAAUAGCAAGUUAAAAUAAGGC | |
| | | CCUG | | UAGUCCGUUAUCAACUUGAAAAAGUGGCA | |
| | | AdAUd | | CCGAGUCGGUGCUsususu | |
| | | AACC | | | |
| | | CUC | | | |
| | GA655 | | | asasdGsAUAdCCUGAdAUdAACCCUCgUUUUA | 158 |
| | | | | GagcuaGaaauagcaaGUUaAaAuAaggcuaGUccGUU | |
| | | | | AucAAcuuGaaaaagugGcaccgagucggugcusususu | |
| Human | GA704 | dAsdAs | 55 | dAsdAsdGsdAdUdAdCdCdUdGAAUAACCCUCG | 159 |
| (29) | | dGsdA | | UUUUAGAGCUAGAAAUAGCAAGUUAAAAU | |
| | | dUdAd | | AAGGCUAGUCCGUUAUCAACUUGAAAAAG | |
| | | CdCdU | | UGGCACCGAGUCGGUGCUsususu | |
| | | dGAA | | | |
| | | UAAC | | | |
| | | CCUC | | | |
| Human | GA705 | asasgsd | 56 | asasgsdAdUdACdCdUdGdAAUAACCCUCGUUU | 160 |
| (30) | | AdUdA | | UAGAGCUAGAAAUAGCAAGUUAAAAUAAG | |
| | | CdCdU | | GCUAGUCCGUUAUCAACUUGAAAAAGUGG | |
| | | dGdAA | | CACCGAGUCGGUGCUsususu | |
| | | UAAC | | | |
| | | CCUC | | | |
| Human | GA706 | asdAsg | 57 | asdAsgsdAdUdAdCdCUGdAdAUAACCCUCGUU | 161 |
| (31) | | sdAdU | | UUAGAGCUAGAAAUAGCAAGUUAAAAUAA | |
| | | dAdCd | | GGCUAGUCCGUUAUCAACUUGAAAAAGUG | |
| | | CUGd | | GCACCGAGUCGGUGCUsususu | |
| | | AdAU | | | |
| | | AACC | | | |
| | | CUC | | | |
| | GA631 | | | asdAsgsdAdUdAdCdCUGdAdAUAACCCUCgUU | 162 |
| | | | | UUAGagcuaGaaauagcaaGUUaAaAuAaggcuaGUcc | |
| | | | | GUUAucAAcuuGaaaaagugGcaccgagucggugcususus | |
| | | | | u | |
| Human | GA707 | asasdG | 58 | asasdGsdAdUdACdCdUdGdAAUAACCCUCGUU | 163 |
| (32) | | sdAdU | | UUAGAGCUAGAAAUAGCAAGUUAAAAUAA | |
| | | dACdC | | GGCUAGUCCGUUAUCAACUUGAAAAAGUG | |
| | | dUdGd | | GCACCGAGUCGGUGCUsususu | |
| | | AAUA | | | |
| | | ACCC | | | |
| | | UC | | | |
| Human | GA708 | asasgsd | 59 | asasgsdAdUdAdCCUGAdAdUAACCCUdCGUUU | 164 |
| (33) | | AdUdA | | UAGAGCUAGAAAUAGCAAGUUAAAAUAAG | |
| | | dCCU | | GCUAGUCCGUUAUCAACUUGAAAAAGUGG | |
| | | GAdAd | | CACCGAGUCGGUGCUsususu | |
| | | UAAC | | | |
| | | CCUdC | | | |
| Human | GA709 | Asasgs | 60 | dAsasgsdAdUdAdCCUGAdAdUAACCCUdCGUU | 165 |
| (34) | | dAdUd | | UUAGAGCUAGAAAUAGCAAGUUAAAAUAA | |
| | | AdCC | | GGCUAGUCCGUUAUCAACUUGAAAAAGUG | |
| | | UGAd | | GCACCGAGUCGGUGCUsususu | |
| | | AdUA | | | |
| | | ACCC | | | |
| | | UdC | | | |
| Human | GA710 | dAsasg | 61 | dAsasgsdAdUAdCdCdUdGdAAdUAACdCCUCGU | 166 |
| (35) | | sdAdU | | UUUAGAGCUAGAAAUAGCAAGUUAAAAUA | |
| | | AdCdC | | AGGCUAGUCCGUUAUCAACUUGAAAAAGU | |
| | | dUdGd | | GGCACCGAGUCGGUGCUsususu | |
| | | AAdU | | | |
| | | AACd | | | |
| | | CCUC | | | |
| Human | GA711 | asasgsd | 62 | asasgsdAdUAdCdCdUdGdAAdUAACdCCUCGUU | 167 |
| (36) | | AdUAd | | UUAGAGCUAGAAAUAGCAAGUUAAAAUAA | |
| | | CdCdU | | GGCUAGUCCGUUAUCAACUUGAAAAAGUG | |
| | | dGdAA | | GCACCGAGUCGGUGCUsususu | |
| | | dUAA | | | |
| | | CdCCU | | | |
| | | C | | | |
| Human | GA712 | asasdG | 63 | asasdGsdAdUdACCUGAAUAACCCUCGUUUUA | 168 |
| (37) | | sdAdU | | GAGCUAGAAAUAGCAAGUUAAAAUAAGGC | |
| | | dACC | | UAGUCCGUUAUCAACUUGAAAAAGUGGCA | |
| | | UGAA | | CCGAGUCGGUGCUsususu | |
| | | UAAC | | | |
| | | CCUC | | | |
| | GA632 | | | asasdGsdAdUdACCUGAAUAACCCUCgUUUUA | 169 |
| | | | | GagcuaGaaauagcaaGUUaAaAuAaggcuaGUccGUU | |
| | | | | AucAAcuuGaaaaagugGcaccgagucggugcusususu | |
| Monkey | GA0749 | asasdG | 64 | asasdGsdAdUdACCUGAAUAACUCUCgUUUUA | 170 |
| (37) | | sdAdU | | GagcuaGaaauagcaaGUUaAaAuAaggcuaGUccGUU | |
| | | dACC | | AucAAcuuGaaaaagugGcaccgagucggugcusususu | |
| | | UGAA | | | |
| | | UAAC | | | |
| | | UCUC | | | |
| Human | GA713 | asdAsd | 65 | asdAsdGsdAdUdACdCUdGAdAUdAAdCCdCUdC | 171 |
| (38) | | GsdAd | | GUUUUAGAGCUAGAAAUAGCAAGUUAAAA | |
| | | UdACd | | UAAGGCUAGUCCGUUAUCAACUUGAAAAA | |
| | | CUdG | | GUGGCACCGAGUCGGUGCUsususu | |
| | | AdAUd | | | |
| | | AAdC | | | |
| | | CdCUd | | | |
| | | C | | | |
| | GA0666 | | | asdAsdGsdAdUdACdCUdGAdAUdAAdCCdCUdC | 172 |
| | | | | gUUUUAGagcuaGaaauagcaaGUUaAaAuAaggcua | |
| | | | | GUccGUUAucAAcuuGaaaaagugGcaccgagucggugc | |
| | | | | usususu | |
| Monkey | GA0711 | asdAsd | 66 | asdAsdGsdAdUdACdCUdGAdAUdAAdCUdCUdC | 173 |
| (38) | | GsdAd | | gUUUUAGagcuaGaaauagcaaGUUaAaAuAaggcua | |
| | | UdACd | | GUccGUUAucAAcuuGaaaaagugGcaccgagucggugc | |
| | | CUdG | | usususu | |
| | | AdAUd | | | |
| | | AAdC | | | |
| | | UdCUd | | | |
| | | C | | | |
| Human | GA714 | dAsdAs | 67 | dAsdAsgsadUdAdCdCdUdGdAAUdAAdCCdCUC | 174 |
| (39) | | gsadUd | | GUUUUAGAGCUAGAAAUAGCAAGUUAAAA | |
| | | AdCdC | | UAAGGCUAGUCCGUUAUCAACUUGAAAAA | |
| | | dUdGd | | GUGGCACCGAGUCGGUGCUsususu | |
| | | AAUd | | | |
| | | AAdC | | | |
| | | CdCUC | | | |
| Human | GA715 | dAsdAs | 68 | dAsdAsgsadUdAdCdCdUdGdAAUdAACdCCU | 175 |
| (39) | | gsadUd | | CGUUUUAGAGCUAGAAAUAGCAAGUUAAAAU | |
| | | AdCdC | | AAGGCUAGUCCGUUAUCAACUUGAAAAAG | |
| | | dUdGd | | UGGCACCGAGUCGGUGCUsususu | |
| | | AAUd | | | |
| | | AACd | | | |
| | | CCUC | | | |
| | GA633 | | | dAsdAsgsadUdAdCdCdUdGdAAUdAACdCCUCg | 176 |
| | | | | UUUUAGagcuaGaaauagcaaGUUaAaAuAaggcuaG | |
| | | | | UccGUUAucAAcuuGaaaaagugGcaccgagucggug | |
| | | | | cusususu | |
| Monkey | N/A | No | | N/A | |
| (40) | | monkey | | | |
| | | sequence | | | |
| | | designed | | | |
| | | and | | | |
| | | evaluated | | | |

Uppercase nucleotides (A, C, G, I and U) indicate ribonucleotides, adenine, guanine, cytosine, inosine, and uracil, respectively and lowercase nucleotides (a, g, c, i and u) indicate 2′-O-methylribonucleotide (2′-OMe) unless otherwise specified. dA, dC and dG indicate 2′-deoxyadenosine, 2′-deoxycytidine and 2′-deoxyguanosine; dU indicates thymidine and/or 2′-deoxyuridine. Underline in gRNA sequence indicate spacer sequence. s: phosphorothioate (PS).

A summary of the spacer sequences and the guide nucleic acids is provided in Table 8.

TABLE 8
Guide nucleic acid sequences

| | Guide | | SEQ | | SEQ |
|---|---|---|---|---|---|
| | nucleic | Protospacer | ID | | ID |
| Gene Target | acid ID | (5′-3′) | NO | Sequence (5′-3′) | NO |
| cANG | GA547 | AAGATACC | 177 | asasgsAUACCUGAAUAACUCUCgUU | 432 |
| PTL3 | | TGAATAAC | | UUAGagcuaGaaauagcaaGUUaAaAuAag | |
| | | TCTC | | gcuaGUccGUUAucAAcuuGaaaaagugGca | |
| | | | | ccgagucggugcusususuUUu | |
| hANG | GA627 | AAGATACC | 178 | asasgsAdUAdCCdUGdAAdUAdACdCC | 433 |
| PTL3 | | TGAATAAC | | dUCgUUUUAGagcuaGaaauagcaaGUUa | |
| | | CCTC | | AaAuAaggcuaGUccGUUAucAAcuuGaa | |
| | | | | aaagugGcaccgagucggugcusususu | |
| hANG | GA628 | AAGATACC | 179 | asasgsdAUdACdCUdGAdAUdAAdCCd | 434 |
| PTL3 | | TGAATAAC | | CUdCgUUUUAGagcuaGaaauagcaaGUU | |
| | | CCTC | | aAaAuAaggcuaGUccGUUAucAAcuuGa | |
| | | | | aaaagugGcaccgagucggugcusususu | |
| hANG | GA629 | AAGATACC | 180 | asasdGsdAdUdACdCUdGAdAUdAAdC | 435 |
| PTL3 | | TGAATAAC | | CdCUdCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA630 | AAGATACC | 181 | asasdGsdAdUdAdCdCdUdGAdAUdAAd | 436 |
| PTL3 | | TGAATAAC | | CCdCUdCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0631 | AAGATACC | 182 | asasgsdAdUdACCUGAAUAACCCUCg | 437 |
| PTL3 | | TGAATAAC | | UUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA632 | AAGATACC | 183 | asasdGsdAdUdACCUGAAUAACCCUC | 438 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA0633 | AAGATACC | 184 | asasgsdAdUdAdCCUGAAUAACCCUCg | 439 |
| PTL3 | | TGAATAAC | | UUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA723 | AAGATACC | 185 | asasdGsdAdUdAdCCUGAAUAACCCU | 440 |
| PTL3 | | TGAATAAC | | CgUUUUAGagcuaGaaauagcaaGUUaAa | |
| | | CCTC | | AuAaggcuaGUccGUUAucAAcuuGaaaaa | |
| | | | | gugGcaccgagucggugcusususu | |
| hANG | GA0635 | AAGATACC | 186 | asasgsdAdUdAdCdCUGAAUAACCCUC | 441 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA636 | AAGATACC | 187 | asasdGsdAdUdAdCdCUGAAUAACCC | 442 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA0637 | AAGATACC | 188 | asasgsdAdUdAdCdCdUGAAUAACCCU | 443 |
| PTL3 | | TGAATAAC | | CgUUUUAGagcuaGaaauagcaaGUUaAa | |
| | | CCTC | | AuAaggcuaGUccGUUAucAAcuuGaaaaa | |
| | | | | gugGcaccgagucggugcusususu | |
| hANG | GA638 | AAGATACC | 189 | asasdGsdAdUdAdCdCdUGAAUAACCC | 444 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA639 | AAGATACC | 190 | asasgsdAdUdAdCdCdUdGAAUAACCC | 445 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA640 | AAGATACC | 191 | asasdGsdAdUdAdCdCdUdGAAUAACC | 446 |
| PTL3 | | TGAATAAC | | CUCgUUUUAGagcuaGaaauagcaaGUUa | |
| | | CCTC | | AaAuAaggcuaGUccGUUAucAAcuuGaa | |
| | | | | aaagugGcaccgagucggugcusususu | |
| hANG | GA641 | AAGATACC | 192 | asasgsAUdACCUGAAUAAdCCCUCgU | 447 |
| PTL3 | | TGAATAAC | | UUUAGagcuaGaaauagcaaGUUaAaAuAa | |
| | | CCTC | | ggcuaGUccGUUAucAAcuuGaaaaagugG | |
| | | | | caccgagucggugcusususu | |
| hANG | GA642 | AAGATACC | 193 | asasgsAUACCUdGAAdUAACCdCUCg | 448 |
| PTL3 | | TGAATAAC | | UUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA643 | AAGATACC | 194 | asasgsdAdUdAdCCdUdGAAdUdAACC | 449 |
| PTL3 | | TGAATAAC | | CUCguuuuagagcuagaaauagcaaguuaaaaua | |
| | | CCTC | | aggcuaguccguuaucaacuugaaaaaguggcaccg | |
| | | | | agucggugcusususu | |
| hANG | GA745 | AAGATACC | 195 | asasdGsdAUdAdCCUGAAUAACCCUC | 450 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA837 | AAGATACC | 196 | asasdGsdAUdAdCdCUGAAUAACCCU | 451 |
| PTL3 | | TGAATAAC | | CgUUUUAGagcuaGaaauagcaaGUUaAa | |
| | | CCTC | | AuAaggcuaGUccGUUAucAAcuuGaaaaa | |
| | | | | gugGcaccgagucggugcusususu | |
| hANG | GA646 | AAGATACC | 197 | asasdGsdAUdAdCdCdUGAAUAACCC | 452 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA647 | AAGATACC | 198 | asasdGsdAUdAdCdCdUdGAAUAACCC | 453 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA648 | AAGATACC | 199 | asasdGsdAUAdCdCdUGAAUAACCCU | 454 |
| PTL3 | | TGAATAAC | | CgUUUUAGagcuaGaaauagcaaGUUaAa | |
| | | CCTC | | AuAaggcuaGUccGUUAucAAcuuGaaaaa | |
| | | | | gugGcaccgagucggugcusususu | |
| hANG | GA649 | AAGATACC | 200 | asasdGsdAUAdCdCdUdGAAUAACCC | 455 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA650 | AAGATACC | 201 | asasdGsAdUAdCCdUGAAUAACCCUC | 456 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA651 | AAGATACC | 202 | asasgsdAUdACdCUdGAAUAACCCUCg | 457 |
| PTL3 | | TGAATAAC | | UUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA652 | AAGATACC | 203 | asasgsAUACCdUGAAdUAACCCUCgU | 458 |
| PTL3 | | TGAATAAC | | UUUAGagcuaGaaauagcaaGUUaAaAuAa | |
| | | CCTC | | ggcuaGUccGUUAucAAcuuGaaaaagugG | |
| | | | | caccgagucggugcusususu | |
| hANG | GA653 | AAGATACC | 204 | asasdGsAUAdCCdUGAAdUAACCCUC | 459 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA654 | AAGATACC | 205 | asasgsAUACCUGAdAUdAACCCUCgU | 460 |
| PTL3 | | TGAATAAC | | UUUAGagcuaGaaauagcaaGUUaAaAuAa | |
| | | CCTC | | ggcuaGUccGUUAucAAcuuGaaaaagugG | |
| | | | | caccgagucggugcusususu | |
| hANG | GA655 | AAGATACC | 206 | asasdGsAUAdCCUGAdAUdAACCCUC | 461 |
| PTL3 | | TGAATAAC | | gUUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA718 | AAGATACC | 207 | asasgsAUA+CCUGAAUAACCCUCguu | 462 |
| PTL3 | | TGAATAAC | | uuagagcuagaaauagcaaguuaaaauaaggcuagu | |
| | | CCTC | | ccguuaucaacuugaaaaaguggcaccgagucggug | |
| | | | | cusususu | |
| hANG | GA719 | AAGATACC | 208 | asasgsAUA+C+CUGAAUAACCCUCgu | 463 |
| PTL3 | | TGAATAAC | | uuuagagcuagaaauagcaaguuaaaauaaggcuag | |
| | | CCTC | | uccguuaucaacuugaaaaaguggcaccgagucggu | |
| | | | | gcusususu | |
| hANG | GA670 | AAGATACC | 209 | asasgsAUACCUGAAUAACZCUCgUU | 464 |
| PTL3/ | | TGAATAAC | | UUAGagcuaGaaauagcaaGUUaAaAuAag | |
| cANG | | CCTC/AAG | | gcuaGUccGUUAucAAcuuGaaaaagugGca | |
| PTL3 | | ATACCTGA | | ccgagucggugcusususu | |
| | | ATAACTCT | | | |
| | | C | | | |
| hANG | GA671 | AAGATACC | 210 | asasgsAZACCZGAAUAACCCUCgUUU | 465 |
| PTL3 | | TGAATAAC | | UAGagcuaGaaauagcaaGUUaAaAuAaggc | |
| | | CCTC/AAG | | uaGUccGUUAucAAcuuGaaaaagugGcacc | |
| | | ATACCTGA | | gagucggugusususu | |
| | | ATAACTCT | | | |
| | | C | | | |
| hANG | GA0660 | TCGGAAG | 211 | ucggasasgsAUACCUGAAZAACZCUCg | 466 |
| PTL3/ | | ATACCTGA | | UUUUAGagcuaGaaauagcaaGUUaAaAu | |
| cANG | | ATAACCCT | | AaggcuaGUccGUUAucAAcuuGaaaaagu | |
| PTL3 | | C/TCGGAA | | gGcaccgagugcusususu | |
| | | GATACCTG | | | |
| | | AATAACTC | | | |
| | | TC | | | |
| hANG | GA673 | AAGATACC | 212 | asasgsAZACCUGAAZAACZCUCgUUU | 467 |
| PTL3/ | | TGAATAAC | | UAGagcuaGaaauagcaaGUUaAaAuAaggc | |
| cANG | | CCTC/AAG | | uaGUccGUUAucAAcuuGaaaaagugGcacc | |
| PTL3 | | ATACCTGA | | gagucggugcusususu | |
| | | ATAACTCT | | | |
| | | C | | | |
| hANG | GA674 | AAGATACC | 213 | asasgsAZACCZGAAUAACZCUCgUUU | 468 |
| PTL3/ | | TGAATAAC | | UAGagcuaGaaauagcaaGUUaAaAuAaggc | |
| cANG | | CCTC/AAG | | uaGUccGUUAucAAcuuGaaaaagugGcacc | |
| PTL3 | | ATACCTGA | | gagucggugcusususu | |
| | | ATAACTCT | | | |
| | | C | | | |
| cANG | GA664 | AAGATACC | 214 | asasdGsdAdUdAdCdCdUdGAdAUdAAd | 469 |
| PTL3 | | TGAATAAC | | CUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0664 | AAGATACC | 215 | asasgsdAdUdAdCdCdUdGAAUAACUC | 470 |
| PTL3 | | TGAATAAC | | UCgUUUUAGagcuaGaaauagcaaGUUaA | |
| | | TCTC | | aAuAaggcuaGUccGUUAucAAcuuGaaaa | |
| | | | | agugGcaccgagucggugcusususu | |
| cANG | GA0665 | AAGATACC | 216 | asasdGsdAdUdAdCdCdUdGAAUAACU | 471 |
| PTL3 | | TGAATAAC | | CUCgUUUUAGagcuaGaaauagcaaGUUa | |
| | | TCTC | | AaAuAaggcuaGUccGUUAucAAcuuGaa | |
| | | | | aaagugGcaccgagucggugcusususu | |
| cANG | GA0666 | AAGATACC | 217 | asdAsdGsdAdUdACdCUdGAdAUdAAd | 472 |
| PTL3 | | TGAATAAC | | CCdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0667 | AAGATACC | 218 | asasdGsdAdUdAdCdCUdGAdAUdAAdC | 473 |
| PTL3 | | TGAATAAC | | CdCUdCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| hANG | GA0668 | AAGATACC | 219 | asasgsdAdUdACdCUdGAdAUdAAdCCd | 474 |
| PTL3 | | TGAATAAC | | CUdCgUUUUAGagcuaGaaauagcaaGUU | |
| | | CCTC | | aAaAuAaggcuaGUccGUUAucAAcuuGa | |
| | | | | aaaagugGcaccgagucggugcusususu | |
| hANG | GA0669 | AAGATACC | 220 | asasdGsdAdUdAdCdCdUdGdAdAUdAA | 475 |
| PTL3 | | TGAATAAC | | dCCdCUdCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0670 | AAGATACC | 221 | asasdGsdAdUdAdCdCdUdGdAdAdUdA | 476 |
| PTL3 | | TGAATAAC | | AdCCdCUdCgUUUUAGagcuaGaaauagc | |
| | | CCTC | | aaGUUaAaAuAaggcuaGUccGUUAucA | |
| | | | | AcuuGaaaaagugGcaccgagucggugcususus | |
| | | | | u | |
| hANG | GA0671 | AAGATACC | 222 | asasdGsdAdUdAdCdCdUdGdAdAdUdA | 477 |
| PTL3 | | TGAATAAC | | dAdCCdCUdCgUUUUAGagcuaGaaauag | |
| | | CCTC | | caaGUUaAaAuAaggcuaGUccGUUAucA | |
| | | | | AcuuGaaaaagugGcaccgagucggugcususus | |
| | | | | u | |
| hANG | GA0672 | AAGATACC | 223 | asasdGsdAdUdAdCdCdUdGdAdAdUdA | 478 |
| PTL3 | | TGAATAAC | | dAdCdCdCUdCgUUUUAGagcuaGaaaua | |
| | | CCTC | | gcaaGUUaAaAuAaggcuaGUccGUUAuc | |
| | | | | AAcuuGaaaaagugGcaccgagucggugcususu | |
| | | | | su | |
| hANG | GA0673 | AAGATACC | 224 | asasdGsdAdUdAdCdCdUdGdAdAdUdA | 479 |
| PTL3 | | TGAATAAC | | dAdCdCdCdUdCgUUUUAGagcuaGaaau | |
| | | CCTC | | agcaaGUUaAaAuAaggcuaGUccGUUAuc | |
| | | | | AAcuuGaaaaagugGcaccgagucggugcususu | |
| | | | | su | |
| hANG | GA0674 | AAGATACC | 225 | asdAsdGsdAdUdAdCdCdUdGdAdAdUd | 480 |
| PTL3 | | TGAATAAC | | AdAdCdCdCdUdCgUUUUAGagcuaGaa | |
| | | CCTC | | auagcaaGUUaAaAuAaggcuaGUccGUUA | |
| | | | | ucAAcuuGaaaaagugGcaccgagucggugcusu | |
| | | | | susu | |
| hANG | GA0675 | AAGATACC | 226 | asasgsdAdUAdCCdUGdAAdUAdACdC | 481 |
| PTL3 | | TGAATAAC | | CdUCgUUUUAGagcuaGaaauagcaaGUU | |
| | | CCTC | | aAaAuAaggcuaGUccGUUAucAAcuuGa | |
| | | | | aaaagugGcaccgagucggugcusususu | |
| hANG | GA0676 | AAGATACC | 227 | asasdGsAdUAdCCdUGdAAdUAdACdC | 482 |
| PTL3 | | TGAATAAC | | CdUCgUUUUAGagcuaGaaauagcaaGUU | |
| | | CCTC | | aAaAuAaggcuaGUccGUUAucAAcuuGa | |
| | | | | aaaagugGcaccgagucggugcusususu | |
| hANG | GA0677 | AAGATACC | 228 | asasdGsdAdUAdCCdUGdAAdUAdACd | 483 |
| PTL3 | | TGAATAAC | | CCdUCgUUUUAGagcuaGaaauagcaaGU | |
| | | CCTC | | UaAaAuAaggcuaGUccGUUAucAAcuuG | |
| | | | | aaaaagugGcaccgagucggugcusususu | |
| hANG | GA0678 | AAGATACC | 229 | asasdGsdAdUdAdCCdUGdAAdUAdACd | 484 |
| PTL3 | | TGAATAAC | | CCdUCgUUUUAGagcuaGaaauagcaaGU | |
| | | CCTC | | UaAaAuAaggcuaGUccGUUAucAAcuuG | |
| | | | | aaaaagugGcaccgagucggugcusususu | |
| hANG | GA0679 | AAGATACC | 230 | asasdGsdAdUdAdCdCdUGdAAdUAdAC | 485 |
| PTL3 | | TGAATAAC | | dCCdUCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA0680 | AAGATACC | 231 | asasdGsdAdUdAdCdCdUdGdAAdUAdA | 486 |
| PTL3 | | TGAATAAC | | CdCCdUCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0681 | AAGATACC | 232 | asasdGsdAdUdAdCdCdUdGdAdAdUAd | 487 |
| PTL3 | | TGAATAAC | | ACdCCdUCgUUUUAGagcuaGaaauagca | |
| | | CCTC | | aGUUaAaAuAaggcuaGUccGUUAucAA | |
| | | | | cuuGaaaaagugGcaccgagucggugcusususu | |
| hANG | GA0682 | AAGATACC | 233 | asasdGsdAdUdAdCdCdUdGdAdAdUdA | 488 |
| PTL3 | | TGAATAAC | | dACdCCdUCgUUUUAGagcuaGaaauagc | |
| | | CCTC | | aaGUUaAaAuAaggcuaGUccGUUAucA | |
| | | | | AcuuGaaaaagugGcaccgagucggugcususus | |
| | | | | u | |
| hANG | GA0683 | AAGATACC | 234 | asasdGsdAdUdAdCdCdUdGdAdAdUdA | 489 |
| PTL3 | | TGAATAAC | | dAdCdCCdUCgUUUUAGagcuaGaaauag | |
| | | CCTC | | caaGUUaAaAuAaggcuaGUccGUUAucA | |
| | | | | AcuuGaaaaagugGcaccgagucggugcususus | |
| | | | | u | |
| hANG | GA0684 | AAGATACC | 235 | asasdGsdAdUdAdCdCdUdGdAdAdUdA | 490 |
| PTL3 | | TGAATAAC | | dAdCdCdCdUCgUUUUAGagcuaGaaaua | |
| | | CCTC | | gcaaGUUaAaAuAaggcuaGUccGUUAuc | |
| | | | | AAcuuGaaaaagugGcaccgagucggugcususu | |
| | | | | su | |
| hANG | GA0685 | AAGATACC | 236 | asasgsdAdUdAdCdCUdGAdAUdAAdCC | 491 |
| PTL3 | | TGAATAAC | | dCUdCgUUUUAGagcuaGaaauagcaaGU | |
| | | CCTC | | UaAaAuAaggcuaGUccGUUAucAAcuuG | |
| | | | | aaaaagugGcaccgagucggugcusususu | |
| hANG | GA0686 | AAGATACC | 237 | asasgsdAdUdAdCdCdUdGAdAUdAAdC | 492 |
| PTL3 | | TGAATAAC | | CdCUdCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA0687 | AAGATACC | 238 | asasgsdAdUdAdCdCdUdGdAdAUdAAd | 493 |
| PTL3 | | TGAATAAC | | CCdCUdCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0688 | AAGATACC | 239 | asasgsdAdUdAdCdCdUdGdAdAdUdAA | 494 |
| PTL3 | | TGAATAAC | | dCCdCUdCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0689 | AAGATACC | 240 | asasgsdAdUdAdCdCdUdGdAdAdUdAd | 495 |
| PTL3 | | TGAATAAC | | AdCCdCUdCgUUUUAGagcuaGaaauagc | |
| | | CCTC | | aaGUUaAaAuAaggcuaGUccGUUAucA | |
| | | | | AcuuGaaaaagugGcaccgagucggugcususus | |
| | | | | u | |
| hANG | GA0690 | AAGATACC | 241 | asasgsdAdUdAdCdCdUdGdAdAdUdAd | 496 |
| PTL3 | | TGAATAAC | | AdCdCdCUdCgUUUUAGagcuaGaaauag | |
| | | CCTC | | caaGUUaAaAuAaggcuaGUccGUUAucA | |
| | | | | AcuuGaaaaagugGcaccgagucggugcususus | |
| | | | | u | |
| hANG | GA0691 | AAGATACC | 242 | asasgsdAdUdAdCdCdUdGdAdAdUdAd | 497 |
| PTL3 | | TGAATAAC | | AdCdCdCdUdCgUUUUAGagcuaGaaaua | |
| | | CCTC | | gcaaGUUaAaAuAaggcuaGUccGUUAuc | |
| | | | | AAcuuGaaaaagugGcaccgagucggugcususu | |
| | | | | su | |
| hANG | GA0692 | AAGATACC | 243 | asasgsdAdUdAdCCdUGdAAdUAdACdC | 498 |
| PTL3 | | TGAATAAC | | CdUCgUUUUAGagcuaGaaauagcaaGUU | |
| | | CCTC | | aAaAuAaggcuaGUccGUUAucAAcuuGa | |
| | | | | aaaagugGcaccgagucggugcusususu | |
| hANG | GA0693 | AAGATACC | 244 | asasgsdAdUdAdCdCdUGdAAdUAdACd | 499 |
| PTL3 | | TGAATAAC | | CCdUCgUUUUAGagcuaGaaauagcaaGU | |
| | | CCTC | | UaAaAuAaggcuaGUccGUUAucAAcuuG | |
| | | | | aaaaagugGcaccgagucggugcusususu | |
| hANG | GA0694 | AAGATACC | 245 | asasgsdAdUdAdCdCdUdGdAAdUAdAC | 500 |
| PTL3 | | TGAATAAC | | dCCdUCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA0695 | AAGATACC | 246 | asasgsdAdUdAdCdCdUdGdAdAdUAdA | 501 |
| PTL3 | | TGAATAAC | | CdCCdUCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA0696 | AAGATACC | 247 | asasgsdAdUdAdCdCdUdGdAdAdUdAd | 502 |
| PTL3 | | TGAATAAC | | ACdCCdUCgUUUUAGagcuaGaaauagca | |
| | | CCTC | | aGUUaAaAuAaggcuaGUccGUUAucAA | |
| | | | | cuuGaaaaagugGcaccgagucggugcusususu | |
| hANG | GA0697 | AAGATACC | 248 | asasgsdAdUdAdCdCdUdGdAdAdUdAd | 503 |
| PTL3 | | TGAATAAC | | AdCdCCdUCgUUUUAGagcuaGaaauagc | |
| | | CCTC | | aaGUUaAaAuAaggcuaGUccGUUAucA | |
| | | | | AcuuGaaaaagugGcaccgagucggugcususus | |
| | | | | u | |
| hANG | GA0698 | AAGATACC | 249 | asasgsdAdUdAdCdCdUdGdAdAdUdAd | 504 |
| PTL3 | | TGAATAAC | | AdCdCdCdUCgUUUUAGagcuaGaaauag | |
| | | CCTC | | caaGUUaAaAuAaggcuaGUccGUUAucA | |
| | | | | AcuuGaaaaagugGcaccgagucggugcususus | |
| | | | | u | |
| hANG | GA0699 | AAGATACC | 250 | asasgsAdUACCUGAAUAdACCCUCgU | 505 |
| PTL3 | | TGAATAAC | | UUUAGagcuaGaaauagcaaGUUaAaAuAa | |
| | | CCTC | | ggcuaGUccGUUAucAAcuuGaaaaagugG | |
| | | | | caccgagucggugcusususu | |
| hANG | GA0700 | AAGATACC | 251 | asasgsAUACCUGdAAUdAACCCdUCg | 506 |
| PTL3 | | TGAATAAC | | UUUUAGagcuaGaaauagcaaGUUaAaAu | |
| | | CCTC | | AaggcuaGUccGUUAucAAcuuGaaaaagu | |
| | | | | gGcaccgagucggugcusususu | |
| hANG | GA441 | AAGATACC | 252 | asasgsAUACCUGAAUAACCCUCgUU | 507 |
| PTL3 | | TGAATAAC | | UUAGagcuaGaaauagcaaGUUaAaAuAag | |
| | | CCTC | | gcuaGUccGUUAucAAcuuGaaaaagugGca | |
| | | | | ccgagucggugcusususu | |
| hANG | GA0702 | AAGATACC | 253 | asasgsAZACCUGAAUAACCCUCgUU | 508 |
| PTL3 | | TGAATAAC | | UUAGagcuaGaaauagcaaGUUaAaAuAag | |
| | | CCTC | | gcuaGUccGUUAucAAcuuGaaaaagugGca | |
| | | | | ccgagucggugcusususu | |
| hANG | GA665 | AAGATACC | 254 | asasdGsdAdUdACdCUdGAdAUdAAdC | 509 |
| PTL3 | | TGAATAAC | | UdCUdCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA0704 | AAGATACC | 255 | asasgsAUACZUGAAUAACCCUCgUU | 510 |
| PTL3 | | TGAATAAC | | UUAGagcuaGaaauagcaaGUUaAaAuAag | |
| | | CCTC | | gcuaGUccGUUAucAAcuuGaaaaagugGca | |
| | | | | ccgagucggugcusususu | |
| hANG | GA0705 | AAGATACC | 256 | asasgsAZACZUGAAUAACCCUCgUUU | 511 |
| PTL3 | | TGAATAAC | | UAGagcuaGaaauagcaaGUUaAaAuAaggc | |
| | | CCTC | | uaGUccGUUAucAAcuuGaaaaagugGcacc | |
| | | | | gagucggugcusususu | |
| hANG | GA0706 | AAGATACC | 257 | asasgsAUACZUGAAUAACZCUCgUU | 512 |
| PTL3/ | | TGAATAAC | | UUAGagcuaGaaauagcaaGUUaAaAuAag | |
| cANG | | CCTC/AAG | | gcuaGUccGUUAucAAcuuGaaaaagugGca | |
| PTL3 | | ATACCTGA | | ccgagucggugcusususu | |
| | | ATAACTCT | | | |
| | | C | | | |
| hANG | GA0707 | AAGATACC | 258 | asasgsAUACZUGAAZAACZCUCgUUU | 513 |
| PTL3/ | | TGAATAAC | | UAGagcuaGaaauagcaaGUUaAaAuAaggc | |
| cANG | | CCTC/AAG | | uaGUccGUUAucAAcuuGaaaaagugGcacc | |
| PTL3 | | ATACCTGA | | gagucggugcusususu | |
| | | ATAACTCT | | | |
| | | C | | | |
| hANG | GA0708 | AAGATACC | 259 | asasgsAZACCUGAAUAACZCUCgUUU | 514 |
| PTL3/ | | TGAATAAC | | UAGagcuaGaaauagcaaGUUaAaAuAaggc | |
| cANG | | CCTC/AAG | | uaGUccGUUAucAAcuuGaaaaagugGcacc | |
| PTL3 | | ATACCTGA | | gagucggugcusususu | |
| | | ATAACTCT | | | |
| | | C | | | |
| cANG | GA0709 | AAGATACC | 260 | asasgsAdUAdCCdUGdAAdUAdACdUC | 515 |
| PTL3 | | TGAATAAC | | dUCgUUUUAGagcuaGaaauagcaaGUUa | |
| | | TCTC | | AaAuAaggcuaGUccGUUAucAAcuuGaa | |
| | | | | aaagugGcaccgagucggugcusususu | |
| cANG | GA0710 | AAGATACC | 261 | asasgsdAUdACdCUdGAdAUdAAdCUd | 516 |
| PTL3 | | TGAATAAC | | CUdCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA0711 | AAGATACC | 262 | asdAsdGsdAdUdACdCUdGAdAUdAAd | 517 |
| PTL3 | | TGAATAAC | | CUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0712 | AAGATACC | 263 | asasdGsdAdUdAdCdCUdGAdAUdAAdC | 518 |
| PTL3 | | TGAATAAC | | UdCUdCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA0713 | AAGATACC | 264 | asasgsdAdUdACdCUdGAdAUdAAdCUd | 519 |
| PTL3 | | TGAATAAC | | CUdCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA0714 | AAGATACC | 265 | asasdGsdAdUdAdCdCdUdGdAdAUdAA | 520 |
| PTL3 | | TGAATAAC | | dCUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0715 | AAGATACC | 266 | asasdGsdAdUdAdCdCdUdGdAdAdUdA | 521 |
| PTL3 | | TGAATAAC | | AdCUdCUdCgUUUUAGagcuaGaaauagc | |
| | | TCTC | | aaGUUaAaAuAaggcuaGUccGUUAucA | |
| | | | | AcuuGaaaaagugGcaccgagucggugcususus | |
| | | | | u | |
| cANG | GA0716 | AAGATACC | 267 | asasdGsdAdUdAdCdCdUdGdAdAdUdA | 522 |
| PTL3 | | TGAATAAC | | dAdCUdCUdCgUUUUAGagcuaGaaauag | |
| | | TCTC | | caaGUUaAaAuAaggcuaGUccGUUAucA | |
| | | | | AcuuGaaaaagugGcaccgagucggugcususus | |
| | | | | u | |
| cANG | GA0716 | AAGATACC | 268 | asasdGsdAdUdAdCdCdUdGdAdAdUdA | 523 |
| PTL3 | | TGAATAAC | | dAdCdUdCUdCgUUUUAGagcuaGaaaua | |
| | | TCTC | | gcaaGUUaAaAuAaggcuaGUccGUUAuc | |
| | | | | AAcuuGaaaaagugGcaccgagucggugcususu | |
| | | | | su | |
| cANG | GA0718 | AAGATACC | 269 | asasdGsdAdUdAdCdCdUdGdAdAdUdA | 524 |
| PTL3 | | TGAATAAC | | dAdCdUdCdUdCgUUUUAGagcuaGaaau | |
| | | TCTC | | agcaaGUUaAaAuAaggcuaGUccGUUAuc | |
| | | | | AAcuuGaaaaagugGcaccgagucggugcususu | |
| | | | | su | |
| cANG | GA0719 | AAGATACC | 270 | asdAsdGsdAdUdAdCdCdUdGdAdAdUd | 525 |
| PTL3 | | TGAATAAC | | AdAdCdUdCdUdCgUUUUAGagcuaGaa | |
| | | TCTC | | auagcaaGUUaAaAuAaggcuaGUccGUUA | |
| | | | | ucAAcuuGaaaaagugGcaccgagucggugcusu | |
| | | | | susu | |
| cANG | GA0720 | AAGATACC | 271 | asasgsdAdUAdCCdUGdAAdUAdACdU | 526 |
| PTL3 | | TGAATAAC | | CdUCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA0721 | AAGATACC | 272 | asasdGsAdUAdCCdUGdAAdUAdACdU | 527 |
| PTL3 | | TGAATAAC | | CdUCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA0722 | AAGATACC | 273 | asasdGsdAdUAdCCdUGdAAdUAdACd | 528 |
| PTL3 | | TGAATAAC | | UCdUCgUUUUAGagcuaGaaauagcaaGU | |
| | | TCTC | | UaAaAuAaggcuaGUccGUUAucAAcuuG | |
| | | | | aaaaagugGcaccgagucggugcusususu | |
| cANG | GA0723 | AAGATACC | 274 | asasdGsdAdUdAdCdCdUGdAAdUAdAC | 529 |
| PTL3 | | TGAATAAC | | dUCdUCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA0724 | AAGATACC | 275 | asasdGsdAdUdAdCdCdUdGdAAdUAdA | 530 |
| PTL3 | | TGAATAAC | | CdUCdUCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0725 | AAGATACC | 276 | asasdGsdAdUdAdCdCdUdGdAdAdUAd | 531 |
| PTL3 | | TGAATAAC | | ACdUCdUCgUUUUAGagcuaGaaauagca | |
| | | TCTC | | aGUUaAaAuAaggcuaGUccGUUAucAA | |
| | | | | cuuGaaaaagugGcaccgagucggugcusususu | |
| cANG | GA0726 | AAGATACC | 277 | asasdGsdAdUdAdCdCdUdGdAdAdUdA | 532 |
| PTL3 | | TGAATAAC | | dACdUCdUCgUUUUAGagcuaGaaauagc | |
| | | TCTC | | aaGUUaAaAuAaggcuaGUccGUUAucA | |
| | | | | AcuuGaaaaagugGcaccgagucggugcususus | |
| | | | | u | |
| cANG | GA0727 | AAGATACC | 278 | asasdGsdAdUdAdCdCdUdGdAdAdUdA | 533 |
| PTL3 | | TGAATAAC | | dAdCdUCdUCgUUUUAGagcuaGaaauag | |
| | | TCTC | | caaGUUaAaAuAaggcuaGUccGUUAucA | |
| | | | | AcuuGaaaaagugGcaccgagucggugcususus | |
| | | | | u | |
| cANG | GA0728 | AAGATACC | 279 | asasdGsdAdUdAdCdCdUdGdAdAdUdA | 534 |
| PTL3 | | TGAATAAC | | dAdCdUdCdUCgUUUUAGagcuaGaaaua | |
| | | TCTC | | gcaaGUUaAaAuAaggcuaGUccGUUAuc | |
| | | | | AAcuuGaaaaagugGcaccgagucggugcususu | |
| | | | | su | |
| cANG | GA0729 | AAGATACC | 280 | asasgsdAdUdAdCdCUdGAdAUdAAdCU | 535 |
| PTL3 | | TGAATAAC | | dCUdCgUUUUAGagcuaGaaauagcaaGU | |
| | | TCTC | | UaAaAuAaggcuaGUccGUUAucAAcuuG | |
| | | | | aaaaagugGcaccgagucggugcusususu | |
| cANG | GA0730 | AAGATACC | 281 | asasgsdAdUdAdCdCdUdGAdAUdAAdC | 536 |
| PTL3 | | TGAATAAC | | UdCUdCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA0731 | AAGATACC | 282 | asasgsdAdUdAdCdCdUdGdAdAUdAAd | 537 |
| PTL3 | | TGAATAAC | | CUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0732 | AAGATACC | 283 | asasgsdAdUdAdCdCdUdGdAdAdUdAA | 538 |
| PTL3 | | TGAATAAC | | dCUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0733 | AAGATACC | 284 | asasgsdAdUdAdCdCdUdGdAdAdUdAd | 539 |
| PTL3 | | TGAATAAC | | AdCUdCUdCgUUUUAGagcuaGaaauagc | |
| | | TCTC | | aaGUUaAaAuAaggcuaGUccGUUAucA | |
| | | | | AcuuGaaaaagugGcaccgagucggugcususus | |
| | | | | u | |
| cANG | GA0734 | AAGATACC | 285 | asasgsdAdUdAdCdCdUdGdAdAdUdAd | 540 |
| PTL3 | | TGAATAAC | | AdCdUdCUdCgUUUUAGagcuaGaaauag | |
| | | TCTC | | caaGUUaAaAuAaggcuaGUccGUUAucA | |
| | | | | AcuuGaaaaagugGcaccgagucggugcususus | |
| | | | | u | |
| cANG | GA0735 | AAGATACC | 286 | asasgsdAdUdAdCdCdUdGdAdAdUdAd | 541 |
| PTL3 | | TGAATAAC | | AdCdUdCdUdCgUUUUAGagcuaGaaaua | |
| | | TCTC | | gcaaGUUaAaAuAaggcuaGUccGUUAuc | |
| | | | | AAcuuGaaaaagugGcaccgagucggugcususu | |
| | | | | su | |
| cANG | GA0736 | AAGATACC | 287 | asasgsdAdUdAdCCdUGdAAdUAdACdU | 542 |
| PTL3 | | TGAATAAC | | CdUCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA0737 | AAGATACC | 288 | asasgsdAdUdAdCdCdUGdAAdUAdACd | 543 |
| PTL3 | | TGAATAAC | | UCdUCgUUUUAGagcuaGaaauagcaaGU | |
| | | TCTC | | UaAaAuAaggcuaGUccGUUAucAAcuuG | |
| | | | | aaaaagugGcaccgagucggugcusususu | |
| cANG | GA0738 | AAGATACC | 289 | asasgsdAdUdAdCdCdUdGdAAdUAdAC | 544 |
| PTL3 | | TGAATAAC | | dUCdUCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA0739 | AAGATACC | 290 | asasgsdAdUdAdCdCdUdGdAdAdUAdA | 545 |
| PTL3 | | TGAATAAC | | CdUCdUCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA0742 | AAGATACC | 291 | asasgsdAdUdAdCdCdUdGdAdAdUdAd | 546 |
| PTL3 | | TGAATAAC | | ACdUCdUCgUUUUAGagcuaGaaauagca | |
| | | TCTC | | aGUUaAaAuAaggcuaGUccGUUAucAA | |
| | | | | cuuGaaaaagugGcaccgagucggugcusususu | |
| cANG | GA0743 | AAGATACC | 292 | asasgsdAdUdAdCdCdUdGdAdAdUdAd | 547 |
| PTL3 | | TGAATAAC | | AdCdUCdUCgUUUUAGagcuaGaaauagc | |
| | | TCTC | | aaGUUaAaAuAaggcuaGUccGUUAucA | |
| | | | | AcuuGaaaaagugGcaccgagucggugcususus | |
| | | | | u | |
| cANG | GA0744 | AAGATACC | 293 | asasgsdAdUdAdCdCdUdGdAdAdUdAd | 548 |
| PTL3 | | TGAATAAC | | AdCdUdCdUCgUUUUAGagcuaGaaauag | |
| | | TCTC | | caaGUUaAaAuAaggcuaGUccGUUAucA | |
| | | | | AcuuGaaaaagugGcaccgagucggugcususus | |
| | | | | u | |
| cANG | GA0745 | AAGATACC | 294 | asasgsAdUACCUGAAUAdACUCUCgU | 549 |
| PTL3 | | TGAATAAC | | UUUAGagcuaGaaauagcaaGUUaAaAuAa | |
| | | TCTC | | ggcuaGUccGUUAucAAcuuGaaaaagugG | |
| | | | | caccgagucggugcusususu | |
| cANG | GA0746 | AAGATACC | 295 | asasgsAUdACCUGAAUAAdCUCUCgU | 550 |
| PTL3 | | TGAATAAC | | UUUAGagcuaGaaauagcaaGUUaAaAuAa | |
| | | TCTC | | ggcuaGUccGUUAucAAcuuGaaaaagugG | |
| | | | | caccgagucggugcusususu | |
| cANG | GA753 | AAGATACC | 296 | asasgsAUACCUdGAAdUAACUdCUCg | 551 |
| PTL3 | | TGAATAAC | | UUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA0748 | AAGATACC | 297 | asasgsdAdUdACCUGAAUAACUCUCg | 552 |
| PTL3 | | TGAATAAC | | UUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA0749 | AAGATACC | 298 | asasdGsdAdUdACCUGAAUAACUCUC | 553 |
| PTL3 | | TGAATAAC | | gUUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA0750 | AAGATACC | 299 | asasgsdAdUdAdCCUGAAUAACUCUC | 554 |
| PTL3 | | TGAATAAC | | gUUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA746 | AAGATACC | 300 | asasdGsdAdUdAdCCUGAAUAACUCU | 555 |
| PTL3 | | TGAATAAC | | CgUUUUAGagcuaGaaauagcaaGUUaAa | |
| | | TCTC | | AuAaggcuaGUccGUUAucAAcuuGaaaaa | |
| | | | | gugGcaccgagucggugcusususu | |
| cANG | GA0752 | AAGATACC | 301 | asasgsdAdUdAdCdCUGAAUAACUCU | 556 |
| PTL3 | | TGAATAAC | | CgUUUUAGagcuaGaaauagcaaGUUaAa | |
| | | TCTC | | AuAaggcuaGUccGUUAucAAcuuGaaaaa | |
| | | | | gugGcaccgagucggugcusususu | |
| cANG | GA0753 | AAGATACC | 302 | asasdGsdAdUdAdCdCUGAAUAACUC | 557 |
| PTL3 | | TGAATAAC | | UCgUUUUAGagcuaGaaauagcaaGUUaA | |
| | | TCTC | | aAuAaggcuaGUccGUUAucAAcuuGaaaa | |
| | | | | agugGcaccgagucggugcusususu | |
| cANG | GA0754 | AAGATACC | 303 | asasgsAZACCUGAAUAACUCUCgUU | 558 |
| PTL3 | | TGAATAAC | | UUAGagcuaGaaauagcaaGUUaAaAuAag | |
| | | TCTC | | gcuaGUccGUUAucAAcuuGaaaaagugGca | |
| | | | | ccgagucggugcusususu | |
| cANG | GA0755 | AAGATACC | 304 | asasgsAUACZUGAAUAACUCUCgUU | 559 |
| PTL3 | | TGAATAAC | | UUAGagcuaGaaauagcaaGUUaAaAuAag | |
| | | TCTC | | gcuaGUccGUUAucAAcuuGaaaaagugGca | |
| | | | | ccgagucggugcusususu | |
| cANG | GA0756 | AAGATACC | 305 | asasgsAZACZUGAAUAACUCUCgUU | 560 |
| PTL3 | | TGAATAAC | | UUAGagcuaGaaauagcaaGUUaAaAuAag | |
| | | TCTC | | gcuaGUccGUUAucAAcuuGaaaaagugGca | |
| | | | | ccgagucggugcusususu | |
| cANG | GA0757 | AAGATACC | 306 | asasgsAZACCZGAAUAACUCUCgUUU | 561 |
| PTL3 | | TGAATAAC | | UAGagcuaGaaauagcaaGUUaAaAuAaggc | |
| | | TCTC | | uaGUccGUUAucAAcuuGaaaaagugGcacc | |
| | | | | gagucggugcusususu | |
| hANG | GA0758 | AAGATACC | 307 | asasgsAUACCUGAAYAACCCUCguuuu | 562 |
| PTL3 | | TGAATAAC | | agagcuagaaauagcaaguuaaaauaaggcuagucc | |
| | | CCTC | | guuaucaacuugaaaaaguggcaccgagucggugc | |
| | | | | usususu | |
| hANG | GA0759 | AAGATACC | 308 | asasgsAUACCUGAA(N1mY)AACCCU | 563 |
| PTL3 | | TGAATAAC | | Cguuuuagagcuagaaauagcaaguuaaaauaagg | |
| | | CCTC | | cuaguccguuaucaacuugaaaaaguggcaccgagu | |
| | | | | cggugcusususu | |
| hANG | GA0760 | AAGATACC | 309 | asasgsAUACCUGAAdWAACCCUCguu | 564 |
| PTL3 | | TGAATAAC | | uuagagcuagaaauagcaaguuaaaauaaggcuagu | |
| | | CCTC | | ccguuaucaacuugaaaaaguggcaccgagucggug | |
| | | | | cusususu | |
| hANG | GA0761 | AAGATACC | 310 | asasgsAUACCUGAATAACCCUCguuuu | 565 |
| PTL3 | | TGAATAAC | | agagcuagaaauagcaaguuaaaauaaggcuagucc | |
| | | CCTC | | guuaucaacuugaaaaaguggcaccgagucggugc | |
| | | | | usususu | |
| hANG | GA0762 | AAGATACC | 311 | asasgsAUACCYGAAUAACCCUCguuuu | 566 |
| PTL3 | | TGAATAAC | | agagcuagaaauagcaaguuaaaauaaggcuagucc | |
| | | CCTC | | guuaucaacuugaaaaaguggcaccgagucggugc | |
| | | | | usususu | |
| hANG | GA0763 | AAGATACC | 312 | asasgsAUACC(N1mY)GAAUAACCCU | 567 |
| PTL3 | | TGAATAAC | | Cguuuuagagcuagaaauagcaaguuaaaauaagg | |
| | | CCTC | | cuaguccguuaucaacuugaaaaaguggcaccgagu | |
| | | | | cggugcusususu | |
| hANG | GA0764 | AAGATACC | 313 | asasgsAUACCdWGAAUAACCCUCguu | 568 |
| PTL3 | | TGAATAAC | | uuagagcuagaaauagcaaguuaaaauaaggcuagu | |
| | | CCTC | | ccguuaucaacuugaaaaaguggcaccgagucggug | |
| | | | | cusususu | |
| hANG | GA0765 | AAGATACC | 314 | asasgsAUACCTdUGAAUAACCCUCgu | 569 |
| PTL3 | | TGAATAAC | | uuuagagcuagaaauagcaaguuaaaauaaggcuag | |
| | | CCTC | | uccguuaucaacuugaaaaaguggcaccgagucggu | |
| | | | | gcusususu | |
| hANG | GA0766 | AAGATACC | 315 | asasgsAUACCYGAAYAACCCUCguuuu | 570 |
| PTL3 | | TGAATAAC | | agagcuagaaauagcaaguuaaaauaaggcuagucc | |
| | | CCTC | | guuaucaacuugaaaaaguggcaccgagucggugc | |
| | | | | usususu | |
| hANG | GA0767 | AAGATACC | 316 | asasgsAUACC(N1mY)GAA(N1mY)AA | 571 |
| PTL3 | | TGAATAAC | | CCCUCguuuuagagcuagaaauagcaaguuaaa | |
| | | CCTC | | auaaggcuaguccguuaucaacuugaaaaaguggca | |
| | | | | ccgagucggugcusususu | |
| hANG | GA0768 | AAGATACC | 317 | asasgsAUACCdWGAAdWAACCCUCgu | 572 |
| PTL3 | | TGAATAAC | | uuuagagcuagaaauagcaaguuaaaauaaggcuag | |
| | | CCTC | | uccguuaucaacuugaaaaaguggcaccgagucggu | |
| | | | | gcusususu | |
| hANG | GA0769 | AAGATACC | 318 | asasgsAUACCTdUGAATdUAACCCUC | 573 |
| PTL3 | | TGAATAAC | | guuuuagagcuagaaauagcaaguuaaaauaaggcu | |
| | | CCTC | | aguccguuaucaacuugaaaaaguggcaccgagucg | |
| | | | | gugcusususu | |
| hANG | GA0770 | AAGATACC | 319 | asasgsAUACCUGA(2,6- | 574 |
| PTL3 | | TGAATAAC | | DAP)UAACCCUCguuuuagagcuagaaauag | |
| | | CCTC | | caaguuaaaauaaggcuaguccguuaucaacuugaa | |
| | | | | aaaguggcaccgagucggugcusususu | |
| hANG | GA0771 | AAGATACC | 320 | asasgsAUACCUGAAU(2,6- | 575 |
| PTL3 | | TGAATAAC | | DAP)ACCCUCguuuuagagcuagaaauagcaa | |
| | | CCTC | | guuaaaauaaggcuaguccguuaucaacuugaaaaa | |
| | | | | guggcaccgagucggugcusususu | |
| hANG | GA0772 | AAGATACC | 321 | asasgsAUACCUGA(2,6-DAP)U(2,6- | 576 |
| PTL3 | | TGAATAAC | | DAP)ACCCUCguuuuagagcuagaaauagcaa | |
| | | CCTC | | guuaaaauaaggcuaguccguuaucaacuugaaaaa | |
| | | | | guggcaccgagucggugcusususu | |
| hANG | GA0773 | AAGATACC | 322 | asasgsAUACCUGAAdUAACCCUCguuu | 577 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0774 | AAGATACC | 323 | asasgsAUACCUGAAdUAACCCUCguuu | 578 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0775 | AAGATACC | 324 | asasgsAdUACCUGAAdUAACCCUCguu | 579 |
| PTL3 | | TGAATAAC | | uuagagcuagaaauagcaaguuaaaauaaggcuagu | |
| | | CCTC | | ccguuaucaacuugaaaaaguggcaccgagucggug | |
| | | | | cusususu | |
| hANG | GA0776 | AAGATACC | 325 | asasgsAUACCUdGAAUAACCCUCguuu | 580 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0777 | AAGATACC | 326 | +As+As+GsAUACCUGAAUAACCCUC | 581 |
| PTL3 | | TGAATAAC | | guuuuagagcuagaaauagcaaguuaaaauaaggcu | |
| | | CCTC | | aguccguuaucaacuugaaaaaguggcaccgagucg | |
| | | | | gugcusususu | |
| hANG | GA0778 | AAGATACC | 327 | asasgsAU+A+CCUGAAUAACCCUCgu | 582 |
| PTL3 | | TGAATAAC | | uuuagagcuagaaauagcaaguuaaaauaaggcuag | |
| | | CCTC | | uccguuaucaacuugaaaaaguggcaccgagucggu | |
| | | | | gcusususu | |
| hANG | GA0779 | AAGATACC | 328 | asasgsdAUACCUGAAUAACCCUCguuu | 583 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0780 | AAGATACC | 329 | asasgsAdUACCUGAAUAACCCUCguuu | 584 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0781 | AAGATACC | 330 | asasgsAUdACCUGAAUAACCCUCguuu | 585 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0782 | AAGATACC | 331 | asasgsAUAdCCUGAAUAACCCUCguuu | 586 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0783 | AAGATACC | 332 | asasgsAUACCdUGAAUAACCCUCguuu | 587 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0784 | AAGATACC | 333 | asasgsAUACCUGAAUdAACCCUCguuu | 588 |
| PTL3 | | TGAATAAC | | uagagcuagaaauagcaaguuaaaauaaggcuaguc | |
| | | CCTC | | cguuaucaacuugaaaaaguggcaccgagucggugc | |
| | | | | usususu | |
| hANG | GA0785 | AAGATACC | 334 | asasgsAUACCUIAAUAACCCUCguuuu | 589 |
| PTL3 | | TGAATAAC | | agagcuagaaauagcaaguuaaaauaaggcuagucc | |
| | | CCTC | | guuaucaacuugaaaaaguggcaccgagucggugc | |
| | | | | usususu | |
| hANG | GA0786 | AAGATACC | 335 | asasgsAUACC(MP- | 590 |
| PTL3 | | TGAATAAC | | U)GAAUAACCCUCguuuuagagcuagaaau | |
| | | CCTC | | agcaaguuaaaauaaggcuaguccguuaucaacuug | |
| | | | | aaaaaguggcaccgagucggugcusususu | |
| hANG | GA0787 | AAGATACC | 336 | asasgsAUACCUGAA(MP- | 591 |
| PTL3 | | TGAATAAC | | U)AACCCUCguuuuagagcuagaaauagcaag | |
| | | CCTC | | uuaaaauaaggcuaguccguuaucaacuugaaaaag | |
| | | | | uggcaccgagucggugcusususu | |
| hANG | GA0788 | AAGATACC | 337 | asasgsAUACC(MP-U)GAA(MP- | 592 |
| PTL3 | | TGAATAAC | | U)AACCCUCguuuuagagcuagaaauagcaag | |
| | | CCTC | | uuaaaauaaggcuaguccguuaucaacuugaaaaag | |
| | | | | uggcaccgagucggugcusususu | |
| hANG | GA472 | AAGATACC | 338 | asasisAUACCUGAAUAACCCUCgUUU | 593 |
| PTL3 | | TGAATAAC | | UAGagcuaGaaauagcaaGUUaAaAuAaggc | |
| | | CCTC | | uaGUccGUUAucAAcuuGaaaaagugGcacc | |
| | | | | gagucggugcusususu | |
| hANG | GA473 | AAGATACC | 339 | asasisAUACCUIAAUAACCCUCgUUU | 594 |
| PTL3 | | TGAATAAC | | UAGagcuaGaaauagcaaGUUaAaAuAaggc | |
| | | CCTC | | uaGUccGUUAucAAcuuGaaaaagugGcacc | |
| | | | | gagucggugcusususu | |
| hANG | GA631 | AAGATACC | 340 | asdAsgsdAdUdAdCdCUGdAdAUAACC | 595 |
| PTL3 | | TGAATAAC | | CUCgUUUUAGagcuaGaaauagcaaGUUa | |
| | | CCTC | | AaAuAaggcuaGUccGUUAucAAcuuGaa | |
| | | | | aaagugGcaccgagucggugcusususu | |
| hANG | GA633 | AAGATACC | 341 | dAsdAsgsadUdAdCdCdUdGdAAUdAA | 596 |
| PTL3 | | TGAATAAC | | CdCCUCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA635 | AAGATACC | 342 | dAsdAsgsadUdAdCdCdUdGdAAUdAAd | 597 |
| PTL3 | | TGAATAAC | | CdCdCUCgUUUUAGagcuaGaaauagcaa | |
| | | CCTC | | GUUaAaAuAaggcuaGUccGUUAucAAc | |
| | | | | uuGaaaaagugGcaccgagucggugcusususu | |
| hANG | GA636 | AAGATACC | 343 | asasdGsdAdUdAdCdCUGAAUAACCC | 598 |
| PTL3 | | TGAATAAC | | UCgUUUUAGagcuaGaaauagcaaGUUaA | |
| | | CCTC | | aAuAaggcuaGUccGUUAucAAcuuGaaaa | |
| | | | | agugGcaccgagucggugcusususu | |
| hANG | GA722 | AAGATACC | 344 | asdAsgsadUdAdCdCdUdGdAAUdAAdC | 599 |
| PTL3 | | TGAATAAC | | dCdCUCgUUUUAGagcuaGaaauagcaaG | |
| | | CCTC | | UUaAaAuAaggcuaGUccGUUAucAAcuu | |
| | | | | GaaaaagugGcaccgagucggugcusususu | |
| hANG | GA672 | AAGATACC | 345 | asasgsAUACCUGAAZAACZCUCgUUU | 600 |
| PTL3/c | | TGAATAAC | | UAGagcuaGaaauagcaaGUUaAaAuAaggc | |
| ANGP | | CCTC/AAG | | uaGUccGUUAucAAcuuGaaaaagugGcacc | |
| TL3 | | ATACCTGA | | gagucggugcusususu | |
| | | ATAACTCT | | | |
| | | C | | | |
| hANG | GA675 | AAGATACC | 346 | asdAsgsadUdAdCdCdUdGdAAUdAAdC | 601 |
| PTL3 | | TGAATAAC | | dCdCUCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| hANG | GA676 | AAGATACC | 347 | asasdGsdAdUdAdCdCdUdGAdAUdAAd | 602 |
| PTL3 | | TGAATAAC | | CCdCUdCGUUUUAGAGCUAGAAAU | |
| | | CCTC | | AGCAAGUUAAAAUAAGGCUAGUC | |
| | | | | CGUUAUCAACUUGAAAAAGUGGC | |
| | | | | ACCGAGUCGGUGCUsususu | |
| hANG | GA677 | AAGATACC | 348 | asasdGsdAdUdACdCUdGAdAUdAAdC | 603 |
| PTL3 | | TGAATAAC | | CdCUdCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| hANG | GA678 | AAGATACC | 349 | asdAsdGsdAdTdAdCdCdTdGAAUAAC | 604 |
| PTL3 | | TGAATAAC | | CCUCGUUUUAGAGCUAGAAAUAG | |
| | | CCTC | | CAAGUUAAAAUAAGGCUAGUCCG | |
| | | | | UUAUCAACUUGAAAAAGUGGCAC | |
| | | | | CGAGUCGGUGCUsususu | |
| hANG | GA679 | AAGATACC | 350 | dAsdAsdGsdAdUdACdCdUdGdAAUAA | 605 |
| PTL3 | | TGAATAAC | | CCCUCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| hANG | GA680 | AAGATACC | 351 | asasgsAUAdCCdUGAAdUAACCCUCG | 606 |
| PTL3 | | TGAATAAC | | UUUUAGAGCUAGAAAUAGCAAGU | |
| | | CCTC | | UAAAAUAAGGCUAGUCCGUUAUC | |
| | | | | AACUUGAAAAAGUGGCACCGAGU | |
| | | | | CGGUGCUsususu | |
| hANG | GA681 | AAGATACC | 352 | asdAsdGsdAdUdAdCdCdUdGAdAUdA | 607 |
| PTL3 | | TGAATAAC | | AdCCdCUdCGUUUUAGAGCUAGAA | |
| | | CCTC | | AUAGCAAGUUAAAAUAAGGCUAG | |
| | | | | UCCGUUAUCAACUUGAAAAAGUG | |
| | | | | GCACCGAGUCGGUGCUsususu | |
| hANG | GA682 | AAGATACC | 353 | asasdGsdAdUdAdCCUGAAUAACCCU | 608 |
| PTL3 | | TGAATAAC | | CGUUUUAGAGCUAGAAAUAGCAA | |
| | | CCTC | | GUUAAAAUAAGGCUAGUCCGUUA | |
| | | | | UCAACUUGAAAAAGUGGCACCGA | |
| | | | | GUCGGUGCUsususu | |
| hANG | GA683 | AAGATACC | 354 | dAsdAsgsadUdAdCdCdUdGdAAUdAAd | 609 |
| PTL3 | | TGAATAAC | | CdCdCUCGUUUUAGAGCUAGAAAU | |
| | | CCTC | | AGCAAGUUAAAAUAAGGCUAGUC | |
| | | | | CGUUAUCAACUUGAAAAAGUGGC | |
| | | | | ACCGAGUCGGUGCUsususu | |
| hANG | GA684 | AAGATACC | 355 | asasdGsdAdUdAdCdCUGAAUAACCC | 610 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA685 | AAGATACC | 356 | asasgsAdUAdCCdUGdAAdUAdACdCC | 611 |
| PTL3 | | TGAATAAC | | dUCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA686 | AAGATACC | 357 | asasdGsdAdUdAdCdCdUGAAUAACCC | 612 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA687 | AAGATACC | 358 | asasgsdAdUdAdCdCdUdGAAUAACCC | 613 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA688 | AAGATACC | 359 | asasdGsdAdUdAdCdCdUdGAAUAACC | 614 |
| PTL3 | | TGAATAAC | | CUCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA689 | AAGATACC | 360 | asasgsAUdACCUGAAUAAdCCCUCGU | 615 |
| PTL3 | | TGAATAAC | | UUUAGAGCUAGAAAUAGCAAGUU | |
| | | CCTC | | AAAAUAAGGCUAGUCCGUUAUCA | |
| | | | | ACUUGAAAAAGUGGCACCGAGUC | |
| | | | | GGUGCUsususu | |
| hANG | GA690 | AAGATACC | 361 | asasgsAUACCUdGAAdUAACCdCUCG | 616 |
| PTL3 | | TGAATAAC | | UUUUAGAGCUAGAAAUAGCAAGU | |
| | | CCTC | | UAAAAUAAGGCUAGUCCGUUAUC | |
| | | | | AACUUGAAAAAGUGGCACCGAGU | |
| | | | | CGGUGCUsususu | |
| hANG | GA691 | AAGATACC | 362 | asasgsdAdUdAdCCdUdGAAdUdAACC | 617 |
| PTL3 | | TGAATAAC | | CUCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA692 | AAGATACC | 363 | asasdGsdAUdAdCCUGAAUAACCCUC | 618 |
| PTL3 | | TGAATAAC | | GUUUUAGAGCUAGAAAUAGCAAG | |
| | | CCTC | | UUAAAAUAAGGCUAGUCCGUUAU | |
| | | | | CAACUUGAAAAAGUGGCACCGAG | |
| | | | | UCGGUGCUsususu | |
| hANG | GA693 | AAGATACC | 364 | asasdGsdAUdAdCdCUGAAUAACCCU | 619 |
| PTL3 | | TGAATAAC | | CGUUUUAGAGCUAGAAAUAGCAA | |
| | | CCTC | | GUUAAAAUAAGGCUAGUCCGUUA | |
| | | | | UCAACUUGAAAAAGUGGCACCGA | |
| | | | | GUCGGUGCUsususu | |
| hANG | GA694 | AAGATACC | 365 | asasdGsdAUdAdCdCdUGAAUAACCC | 620 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA695 | AAGATACC | 366 | asasdGsdAUdAdCdCdUdGAAUAACCC | 621 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA696 | AAGATACC | 367 | asasdGsdAUAdCdCdUGAAUAACCCU | 622 |
| PTL3 | | TGAATAAC | | CGUUUUAGAGCUAGAAAUAGCAA | |
| | | CCTC | | GUUAAAAUAAGGCUAGUCCGUUA | |
| | | | | UCAACUUGAAAAAGUGGCACCGA | |
| | | | | GUCGGUGCUsususu | |
| hANG | GA697 | AAGATACC | 368 | asasdGsdAUAdCdCdUdGAAUAACCC | 623 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA698 | AAGATACC | 369 | asasdGsAdUAdCCdUGAAUAACCCUC | 624 |
| PTL3 | | TGAATAAC | | GUUUUAGAGCUAGAAAUAGCAAG | |
| | | CCTC | | UUAAAAUAAGGCUAGUCCGUUAU | |
| | | | | CAACUUGAAAAAGUGGCACCGAG | |
| | | | | UCGGUGCUsususu | |
| hANG | GA699 | AAGATACC | 370 | asasgsdAUdACdCUdGAAUAACCCUC | 625 |
| PTL3 | | TGAATAAC | | GUUUUAGAGCUAGAAAUAGCAAG | |
| | | CCTC | | UUAAAAUAAGGCUAGUCCGUUAU | |
| | | | | CAACUUGAAAAAGUGGCACCGAG | |
| | | | | UCGGUGCUsususu | |
| hANG | GA700 | AAGATACC | 371 | asasgsAUACCdUGAAdUAACCCUCGU | 626 |
| PTL3 | | TGAATAAC | | UUUAGAGCUAGAAAUAGCAAGUU | |
| | | CCTC | | AAAAUAAGGCUAGUCCGUUAUCA | |
| | | | | ACUUGAAAAAGUGGCACCGAGUC | |
| | | | | GGUGCUsususu | |
| hANG | GA701 | AAGATACC | 372 | asasgsdAUdACdCUdGAdAUdAAdCCd | 627 |
| PTL3 | | TGAATAAC | | CUdCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA702 | AAGATACC | 373 | asasgsAUACCUGAdAUdAACCCUCGU | 628 |
| PTL3 | | TGAATAAC | | UUUAGAGCUAGAAAUAGCAAGUU | |
| | | CCTC | | AAAAUAAGGCUAGUCCGUUAUCA | |
| | | | | ACUUGAAAAAGUGGCACCGAGUC | |
| | | | | GGUGCUsususu | |
| hANG | GA703 | AAGATACC | 374 | asasdGsAUAdCCUGAdAUdAACCCUC | 629 |
| PTL3 | | TGAATAAC | | GUUUUAGAGCUAGAAAUAGCAAG | |
| | | CCTC | | UUAAAAUAAGGCUAGUCCGUUAU | |
| | | | | CAACUUGAAAAAGUGGCACCGAG | |
| | | | | UCGGUGCUsususu | |
| hANG | GA704 | AAGATACC | 375 | dAsdAsdGsdAdUdAdCdCdUdGAAUAA | 630 |
| PTL3 | | TGAATAAC | | CCCUCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| hANG | GA705 | AAGATACC | 376 | asasgsdAdUdACdCdUdGdAAUAACCC | 631 |
| PTL3 | | TGAATAAC | | UCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA706 | AAGATACC | 377 | asdAsgsdAdUdAdCdCUGdAdAUAACC | 632 |
| PTL3 | | TGAATAAC | | CUCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA707 | AAGATACC | 378 | asasdGsdAdUdACdCdUdGdAAUAACC | 633 |
| PTL3 | | TGAATAAC | | CUCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA708 | AAGATACC | 379 | asasgsdAdUdAdCCUGAdAdUAACCCU | 634 |
| PTL3 | | TGAATAAC | | dCGUUUUAGAGCUAGAAAUAGCAA | |
| | | CCTC | | GUUAAAAUAAGGCUAGUCCGUUA | |
| | | | | UCAACUUGAAAAAGUGGCACCGA | |
| | | | | GUCGGUGCUsususu | |
| hANG | GA709 | AAGATACC | 380 | dAsasgsdAdUdAdCCUGAdAdUAACCC | 635 |
| PTL3 | | TGAATAAC | | UdCGUUUUAGAGCUAGAAAUAGCA | |
| | | CCTC | | AGUUAAAAUAAGGCUAGUCCGUU | |
| | | | | AUCAACUUGAAAAAGUGGCACCG | |
| | | | | AGUCGGUGCUsususu | |
| hANG | GA710 | AAGATACC | 381 | dAsasgsdAdUAdCdCdUdGdAAdUAAC | 636 |
| PTL3 | | TGAATAAC | | dCCUCGUUUUAGAGCUAGAAAUAG | |
| | | CCTC | | CAAGUUAAAAUAAGGCUAGUCCG | |
| | | | | UUAUCAACUUGAAAAAGUGGCAC | |
| | | | | CGAGUCGGUGCUsususu | |
| hANG | GA711 | AAGATACC | 382 | asasgsdAdUAdCdCdUdGdAAdUAACdC | 637 |
| PTL3 | | TGAATAAC | | CUCGUUUUAGAGCUAGAAAUAGC | |
| | | CCTC | | AAGUUAAAAUAAGGCUAGUCCGU | |
| | | | | UAUCAACUUGAAAAAGUGGCACC | |
| | | | | GAGUCGGUGCUsususu | |
| hANG | GA712 | AAGATACC | 383 | asasdGsdAdUdACCUGAAUAACCCUC | 638 |
| PTL3 | | TGAATAAC | | GUUUUAGAGCUAGAAAUAGCAAG | |
| | | CCTC | | UUAAAAUAAGGCUAGUCCGUUAU | |
| | | | | CAACUUGAAAAAGUGGCACCGAG | |
| | | | | UCGGUGCUsususu | |
| hANG | GA713 | AAGATACC | 384 | asdAsdGsdAdUdACdCUdGAdAUdAAd | 639 |
| PTL3 | | TGAATAAC | | CCdCUdCGUUUUAGAGCUAGAAAU | |
| | | CCTC | | AGCAAGUUAAAAUAAGGCUAGUC | |
| | | | | CGUUAUCAACUUGAAAAAGUGGC | |
| | | | | ACCGAGUCGGUGCUsususu | |
| hANG | GA714 | AAGATACC | 385 | dAsdAsgsadUdAdCdCdUdGdAAUdAAd | 640 |
| PTL3 | | TGAATAAC | | CCdCUCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| hANG | GA715 | AAGATACC | 386 | dAsdAsgsadUdAdCdCdUdGdAAUdAA | 641 |
| PTL3 | | TGAATAAC | | CdCCUCGUUUUAGAGCUAGAAAUA | |
| | | CCTC | | GCAAGUUAAAAUAAGGCUAGUCC | |
| | | | | GUUAUCAACUUGAAAAAGUGGCA | |
| | | | | CCGAGUCGGUGCUsususu | |
| cANG | GA720 | AGAAAAA | 387 | asgsaasaaAGAUACCUGAAUAACUCU | 642 |
| PTL3 | | GATACCTG | | CgUUUUAGagcuaGaaauagcaaGUUaAa | |
| | | AATAACTC | | AuAaggcuaGUccGUUAucAAcuuGaaaaa | |
| | | TC | | gugGcaccgagucggugcusususu | |
| cANG | GA721 | TCAGAAA | 388 | uscsagsaaAAAGAUACCUGAAUAACU | 643 |
| PTL3 | | AAGATACC | | CUCgUUUUAGagcuaGaaauagcaaGUUa | |
| | | TGAATAAC | | AaAuAaggcuaGUccGUUAucAAcuuGaa | |
| | | TCTC | | aaagugGcaccgagucggugcusususu | |
| hANG | GA724 | AAGATACC | 389 | asasgsdAdUdAdCCdUdGAAdUdAACC | 644 |
| PTL3 | | TGAATAAC | | CUCgUUUUAGagcuaGaaauagcaaGUUa | |
| | | CCTC | | AaAuAaggcuaGUccGUUAucAAcuuGaa | |
| | | | | aaagugGcaccgagucggugcusususu | |
| hANG | GA735 | AAGATACC | 390 | GGAAGAUACCUGAAUAACCCUCGU | 645 |
| PTL3 | | TGAATAAC | | UUUAGAGCUAGAAAUAGCAAGUU | |
| | | CCTC | | AAAAUAAGGCUAGUCCGUUAUCA | |
| | | | | ACUUGAAAAAGUGGCACCGAGUC | |
| | | | | GGUGCUUUU | |
| hANG | GA741 | AAGATACC | 39 | GAAGAUACCUGAAUAACCCUCGUU | 646 |
| PTL3 | | TGAATAAC | | UUAGAGCUAGAAAUAGCAAGUUA | |
| | | CCTC | | AAAUAAGGCUAGUCCGUUAUCAA | |
| | | | | CUUGAAAAAGUGGCACCGAGUCG | |
| | | | | GUGCUUUU | |
| cANG | GA344 | aagatacCTG | 392 | asasgsAUACCUGAAUAACUCUCGUU | 647 |
| PTL3 | | AATAACTC | | UUAGAGCUAGAAAUAGCAAGUUA | |
| | | TC | | AAAUAAGGCUAGUCCGUUAUCAA | |
| | | | | CUUGAAAAAGUGGCACCGAGUCG | |
| | | | | GUGCUsususu | |
| cANG | GA557 | AAGATACC | 393 | asasgsAUACCUGAAUAACUCUCgUU | 648 |
| PTL3 | | TGAATAAC | | UUAGagcuaGaaauagcaaGUUaAaAuAag | |
| | | TCTC | | gcuaGUccGUUAucAAcuuGaaaaaguggca | |
| | | | | ccgagucggugcusususuUUu | |
| cANG | GA663 | AAGATACC | 394 | asdAsgsadUdAdCdCdUdGdAAUdAAdC | 649 |
| PTL3 | | TGAATAAC | | dUdCUCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA664 | AAGATACC | 395 | asasdGsdAdUdAdCdCdUdGAdAUdAAd | 650 |
| PTL3 | | TGAATAAC | | CUdCUdCgUUUUAGagcuaGaaauagcaa | |
| | | TCTC | | GUUaAaAuAaggcuaGUccGUUAucAAc | |
| | | | | uuGaaaaagugGcaccgagucggugcusususu | |
| cANG | GA665 | AAGATACC | 396 | asasdGsdAdUdACdCUdGAdAUdAAdC | 651 |
| PTL3 | | TGAATAAC | | UdCUdCgUUUUAGagcuaGaaauagcaaG | |
| | | TCTC | | UUaAaAuAaggcuaGUccGUUAucAAcuu | |
| | | | | GaaaaagugGcaccgagucggugcusususu | |
| cANG | GA666 | AAGATACC | 397 | asdAsdGsdAdUdAdCdCdUdGAAUAAC | 652 |
| PTL3 | | TGAATAAC | | UCUCgUUUUAGagcuaGaaauagcaaGUU | |
| | | TCTC | | aAaAuAaggcuaGUccGUUAucAAcuuGa | |
| | | | | aaaagugGcaccgagucggugcusususu | |
| cANG | GA667 | AAGATACC | 398 | dAsdAsdGsdAdUdACdCdUdGdAAUAA | 653 |
| PTL3 | | TGAATAAC | | CUCUCgUUUUAGagcuaGaaauagcaaGU | |
| | | TCTC | | UaAaAuAaggcuaGUccGUUAucAAcuuG | |
| | | | | aaaaagugGcaccgagucggugcusususu | |
| cANG | GA668 | AAGATACC | 399 | asasgsAUAdCCdUGAAdUAACUCUCg | 654 |
| PTL3 | | TGAATAAC | | UUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA669 | AAGATACC | 400 | asdAsdGsdAdUdAdCdCdUdGAdAUdA | 655 |
| PTL3 | | TGAATAAC | | AdCUdCUdCgUUUUAGagcuaGaaauagc | |
| | | TCTC | | aaGUUaAaAuAaggcuaGUccGUUAucA | |
| | | | | AcuuGaaaaagugGcaccgagucggugcususus | |
| | | | | u | |
| cANG | GA734 | AAGATACC | 401 | GGAAGAUACCUGAAUAACUCUCG | 656 |
| PTL3 | | TGAATAAC | | UUUUAGAGCUAGAAAUAGCAAGU | |
| | | TCTC | | UAAAAUAAGGCUAGUCCGUUAUC | |
| | | | | AACUUGAAAAAGUGGCACCGAGU | |
| | | | | CGGUGCUUUU | |
| cANG | GA740 | AAGATACC | 402 | GAAGAUACCUGAAUAACUCUCGU | 657 |
| PTL3 | | TGAATAAC | | UUUAGAGCUAGAAAUAGCAAGUU | |
| | | TCTC | | AAAAUAAGGCUAGUCCGUUAUCA | |
| | | | | ACUUGAAAAAGUGGCACCGAGUC | |
| | | | | GGUGCUUUU | |
| cANG | GA747 | AAGATACC | 403 | asasgsdAdUdAdCCdUdGAAdUdAACU | 658 |
| PTL3 | | TGAATAAC | | CUCgUUUUAGagcuaGaaauagcaaGUUa | |
| | | TCTC | | AaAuAaggcuaGUccGUUAucAAcuuGaa | |
| | | | | aaagugGcaccgagucggugcusususu | |
| cANG | GA748 | AAGATACC | 404 | asasdGsdAUdAdCCUGAAUAACUCUC | 659 |
| PTL3 | | TGAATAAC | | gUUUUAGagcuaGaaauagcaaGUUaAaAu | |
| | | TCTC | | AaggcuaGUccGUUAucAAcuuGaaaaagu | |
| | | | | gGcaccgagucggugcusususu | |
| cANG | GA749 | AAGATACC | 405 | asasdGsdAUdAdCdCUGAAUAACUCU | 660 |
| PTL3 | | TGAATAAC | | CgUUUUAGagcuaGaaauagcaaGUUaAa | |
| | | TCTC | | AuAaggcuaGUccGUUAucAAcuuGaaaaa | |
| | | | | gugGcaccgagucggugcusususu | |
| cANG | GA750 | AAGATACC | 406 | asasdGsdAdUdAdCdCdUGAAUAACUC | 661 |
| PTL3 | | TGAATAAC | | UCgUUUUAGagcuaGaaauagcaaGUUaA | |
| | | TCTC | | aAuAaggcuaGUccGUUAucAAcuuGaaaa | |
| | | | | agugGcaccgagucggugcusususu | |
| cANG | GA751 | AAGATACC | 407 | asasdGsdAUdAdCdCdUGAAUAACUC | 662 |
| PTL3 | | TGAATAAC | | UCgUUUUAGagcuaGaaauagcaaGUUaA | |
| | | TCTC | | aAuAaggcuaGUccGUUAucAAcuuGaaaa | |
| | | | | agugGcaccgagucggugcusususu | |
| cANG | GA752 | AAGATACC | 408 | asasgAsdUAdCCdUGdAAdUAdACdUC | 663 |
| PTL3 | | TGAATAAC | | dUCgUUUUAGagcuaGaaauagcaaGUUa | |
| | | TCTC | | AaAuAaggcuaGUccGUUAucAAcuuGaa | |
| | | | | aaagugGcaccgagucggugcusususu | |
| cANG | GA755 | AAGATACC | 409 | asasdGsdAdUdAdCCUGAAUAACUCU | 664 |
| PTL3 | | TGAATAAC | | CGUUUUAGAGCUAGAAAUAGCAA | |
| | | TCTC | | GUUAAAAUAAGGCUAGUCCGUUA | |
| | | | | UCAACUUGAAAAAGUGGCACCGA | |
| | | | | GUCGGUGCUsususu | |
| cANG | GA756 | AAGATACC | 410 | asasgsAUACCUdGAAdUAACUdCUCG | 665 |
| PTL3 | | TGAATAAC | | UUUUAGAGCUAGAAAUAGCAAGU | |
| | | TCTC | | UAAAAUAAGGCUAGUCCGUUAUC | |
| | | | | AACUUGAAAAAGUGGCACCGAGU | |
| | | | | CGGUGCUsususu | |
| cANG | GA757 | AAGATACC | 411 | asasgsdAdUdAdCCdUdGAAdUdAACU | 666 |
| PTL3 | | TGAATAAC | | CUCGUUUUAGAGCUAGAAAUAGC | |
| | | TCTC | | AAGUUAAAAUAAGGCUAGUCCGU | |
| | | | | UAUCAACUUGAAAAAGUGGCACC | |
| | | | | GAGUCGGUGCUsususu | |
| cANG | GA758 | AAGATACC | 412 | asasdGsdAUdAdCCUGAAUAACUCUC | 667 |
| PTL3 | | TGAATAAC | | GUUUUAGAGCUAGAAAUAGCAAG | |
| | | TCTC | | UUAAAAUAAGGCUAGUCCGUUAU | |
| | | | | CAACUUGAAAAAGUGGCACCGAG | |
| | | | | UCGGUGCUsususu | |
| cANG | GA759 | AAGATACC | 413 | asasdGsdAUdAdCdCUGAAUAACUCU | 668 |
| PTL3 | | TGAATAAC | | CGUUUUAGAGCUAGAAAUAGCAA | |
| | | TCTC | | GUUAAAAUAAGGCUAGUCCGUUA | |
| | | | | UCAACUUGAAAAAGUGGCACCGA | |
| | | | | GUCGGUGCUsususu | |
| cANG | GA760 | AAGATACC | 414 | asasdGsdAdUdAdCdCdUGAAUAACUC | 669 |
| PTL3 | | TGAATAAC | | UCGUUUUAGAGCUAGAAAUAGCA | |
| | | TCTC | | AGUUAAAAUAAGGCUAGUCCGUU | |
| | | | | AUCAACUUGAAAAAGUGGCACCG | |
| | | | | AGUCGGUGCUsususu | |
| cANG | GA761 | AAGATACC | 415 | asasdGsdAUdAdCdCdUGAAUAACUC | 670 |
| PTL3 | | TGAATAAC | | UCGUUUUAGAGCUAGAAAUAGCA | |
| | | TCTC | | AGUUAAAAUAAGGCUAGUCCGUU | |
| | | | | AUCAACUUGAAAAAGUGGCACCG | |
| | | | | AGUCGGUGCUsususu | |
| cANG | GA762 | AAGATACC | 416 | asdAsgsadUdAdCdCdUdGdAAUdAAdC | 671 |
| PTL3 | | TGAATAAC | | dUdCUCGUUUUAGAGCUAGAAAUA | |
| | | TCTC | | GCAAGUUAAAAUAAGGCUAGUCC | |
| | | | | GUUAUCAACUUGAAAAAGUGGCA | |
| | | | | CCGAGUCGGUGCUsususu | |
| cANG | GA763 | AAGATACC | 417 | asasdGsdAdUdACdCUdGAdAUdAAdC | 672 |
| PTL3 | | TGAATAAC | | UdCUdCGUUUUAGAGCUAGAAAUA | |
| | | TCTC | | GCAAGUUAAAAUAAGGCUAGUCC | |
| | | | | GUUAUCAACUUGAAAAAGUGGCA | |
| | | | | CCGAGUCGGUGCUsususu | |
| cANG | GA764 | AAGATACC | 418 | asdAsdGsdAdTdAdCdCdTdGAAUAAC | 673 |
| PTL3 | | TGAATAAC | | UCUCGUUUUAGAGCUAGAAAUAG | |
| | | TCTC | | CAAGUUAAAAUAAGGCUAGUCCG | |
| | | | | UUAUCAACUUGAAAAAGUGGCAC | |
| | | | | CGAGUCGGUGCUsususu | |
| cANG | GA765 | AAGATACC | 419 | asasgsAUAdCCdUGAAdUAACUCUCG | 674 |
| PTL3 | | TGAATAAC | | UUUUAGAGCUAGAAAUAGCAAGU | |
| | | TCTC | | UAAAAUAAGGCUAGUCCGUUAUC | |
| | | | | AACUUGAAAAAGUGGCACCGAGU | |
| | | | | CGGUGCUsususu | |
| cANG | GA766 | AAGATACC | 420 | dAsdAsgsadUdAdCdCdUdGdAAUdAAd | 675 |
| PTL3 | | TGAATAAC | | CdUdCUCGUUUUAGAGCUAGAAAU | |
| | | TCTC | | AGCAAGUUAAAAUAAGGCUAGUC | |
| | | | | CGUUAUCAACUUGAAAAAGUGGC | |
| | | | | ACCGAGUCGGUGCUsususu | |
| cANG | GA767 | AAGATACC | 421 | asasgsAdUAdCCdUGdAAdUAdACdUC | 676 |
| PTL3 | | TGAATAAC | | dUCGUUUUAGAGCUAGAAAUAGCA | |
| | | TCTC | | AGUUAAAAUAAGGCUAGUCCGUU | |
| | | | | AUCAACUUGAAAAAGUGGCACCG | |
| | | | | AGUCGGUGCUsususu | |
| cANG | GA768 | AAGATACC | 422 | asasgsdAdUdAdCdCdUdGAAUAACUC | 677 |
| PTL3 | | TGAATAAC | | UCGUUUUAGAGCUAGAAAUAGCA | |
| | | TCTC | | AGUUAAAAUAAGGCUAGUCCGUU | |
| | | | | AUCAACUUGAAAAAGUGGCACCG | |
| | | | | AGUCGGUGCUsususu | |
| cANG | GA769 | AAGATACC | 423 | asasdGsdAdUdAdCdCdUdGAAUAACU | 678 |
| PTL3 | | TGAATAAC | | CUCGUUUUAGAGCUAGAAAUAGC | |
| | | TCTC | | AAGUUAAAAUAAGGCUAGUCCGU | |
| | | | | UAUCAACUUGAAAAAGUGGCACC | |
| | | | | GAGUCGGUGCUsususu | |
| cANG | GA770 | AAGATACC | 424 | asasgsAUdACCUGAAUAAdCUCUCGU | 679 |
| PTL3 | | TGAATAAC | | UUUAGAGCUAGAAAUAGCAAGUU | |
| | | TCTC | | AAAAUAAGGCUAGUCCGUUAUCA | |
| | | | | ACUUGAAAAAGUGGCACCGAGUC | |
| | | | | GGUGCUsususu | |
| hANG | GA656 | AAAGATAC | 425 | asasasGAUACCUGAAUAACCCUCgU | 680 |
| PTL3 | | CTGAATAA | | UUUAGagcuaGaaauagcaaGUUaAaAuAa | |
| | | CCCTC | | ggcuaGUccGUUAucAAcuuGaaaaagugG | |
| | | | | caccgagucggugcusususu | |
| hANG | GA657 | AAAAGATA | 426 | asasaasGAUACCUGAAUAACCCUCgU | 681 |
| PTL3 | | CCTGAATA | | UUUAGagcuaGaaauagcaaGUUaAaAuAa | |
| | | ACCCTC | | ggcuaGUccGUUAucAAcuuGaaaaagugG | |
| | | | | caccgagucggugcusususu | |
| hANG | GA658 | AAAAAGAT | 427 | asasaasaGAUACCUGAAUAACCCUCg | 682 |
| PTL3 | | ACCTGAAT | | UUUUAGagcuaGaaauagcaaGUUaAaAu | |
| | | AACCCTC | | AaggcuaGUccGUUAucAAcuuGaaaaagu | |
| | | | | gGcaccgagucggugcusususu | |
| hANG | GA659 | GAAAAAG | 428 | gsasaasaAGAUACCUGAAUAACCCUC | 683 |
| PTL3 | | ATACCTGA | | gUUUUAGagcuaGaaauagcaaGUUaAaAu | |
| | | ATAACCCT | | AaggcuaGUccGUUAucAAcuuGaaaaagu | |
| | | C | | gGcaccgagucggugcusususu | |
| hANG | GA660 | AGAAAAA | 429 | asgsaasaaAGAUACCUGAAUAACCCU | 684 |
| PTL3 | | GATACCTG | | CgUUUUAGagcuaGaaauagcaaGUUaAa | |
| | | AATAACCC | | AuAaggcuaGUccGUUAucAAcuuGaaaaa | |
| | | TC | | gugGcaccgagucggugcusususu | |
| hANG | GA661 | TCAGAAA | 430 | uscsagsaaAAAGAUACCUGAAUAACC | 685 |
| PTL3 | | AAGATACC | | CUCgUUUUAGagcuaGaaauagcaaGUUa | |
| | | TGAATAAC | | AaAuAaggcuaGUccGUUAucAAcuuGaa | |
| | | CCTC | | aaagugGcaccgagucggugcusususu | |
| hANG | GA662 | TATCAGAA | 431 | usasucsagAAAAAGAUACCUGAAUAA | 686 |
| PTL3 | | AAAGATAC | | CCCUCgUUUUAGagcuaGaaauagcaaGU | |
| | | CTGAATAA | | UaAaAuAaggcuaGUccGUUAucAAcuuG | |
| | | CCCTC | | aaaaagugGcaccgagucggugcusususu | |

Uppercase nucleotides (A, G, C, I and T) indicate 2′-deoxyribonucleotides, adenine, guanine, cytosine, inosine, and thymine, respectively; in Guide RNA sequence: uppercase nucleotides (A, C, G, I and U) indicate ribonucleotides, adenine, guanine, cytosine, inosine, and uracil, respectively and lowercase nucleotides (a, g, c, i and u) indicate 2′-O-methylribonucleotide (2′-OMe) unless otherwise specified.
It is understood that the DNA protospacer is converted to RNA or RNA-equivalent in the guide RNA design except when other modification including 2′-deoxyribonucleotides are introduced into the protospacer section of the single guide RNA; s: phosphorothioate (PS).
dA, dC and dG indicate 2′-deoxyadenosine, 2′-deoxycytidine and 2′-deoxyguanosine; dU indicates thymidine and/or 2′-deoxyuridine, and Z indicate zebularine or 4-deoxyuridine/4-deazacytidine.
Y or Y indicates pseudouridine, N1mY or N1mY indicates N1m-pseudouridine, MP-U indicates 2′-O-methyl-3′-phosphonoacetate-uridine, dW indicates 2′-deoxyethynylmethylpyridone-cytidine, 2,6-DAP indicates 2,6-diaminopurine, and a ″+″ symbol (+A, +G, +C, +U) indicate locked nucleic acids.
X is nebularine.

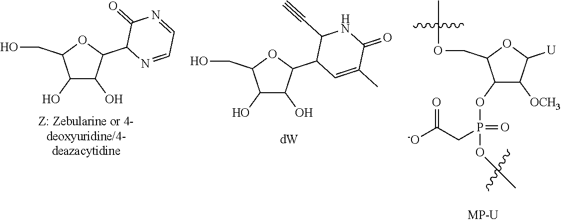

A summary of tracrRNA sequences is provided in Table 9.

TABLE 9
TracrRNA sequences

| | Sequence | Description | SEQ ID NO. |
|---|---|---|---|
| tracrRNA | GUUUUAGAGCUAGAAAUAGCAAGUUAA | Minimally | 3 |
| #1 | AAUAAGGCUAGUCCGUUAUCAACUUGAA | modified | |
| | AAAGUGGCACCGAGUCGGUGCUsususu | tracrRNA | |
| tracrRNA | gUUUUAGagcuaGaaauagcaaGUUaAaAuAaggc | 2′-OMe-enriched | 4 |
| #2 | uaGUccGUUAucAAcuuGaaaaagugGcaccgagucg | tracrRNA | |
| | gugcusususu | | |

A summary of gene editor proteins is provided in Table 10.

TABLE 10
Sequences of gene editor proteins and nucleic acids encoding thereof.

| | | |
|---|---|---|
| | Des- | SEQ ID |
| Sequences | cription | NO. |

MA004

| | | | |
|---|---|---|---|
| MA004 | AGGAAAu′AAGAGAGAAAAGAAGAGu′AAGAAGAAAu′ | mRNA | 687 |
| | Au′AAGAGCCACCAu′GAGCGAGGu′GGAGu′u′CAGCCA | | |
| | CGAGu′ACu′GGAu′GCGGCACGCCCu′GACCCu′GGCCA | | |
| | AGCGGGCCCGGGACGAGCGGGAGGu′GCCCGu′GGGCG | | |
| | CCGu′GCu′GGu′GCu′GAACAACCGGGu′GAu′CGGCGAG | | |
| | GGCu′GGAACCGGGCCAu′CGGCCu′GCACGACCCCACC | | |
| | GCCCACGCCGAGAu′CAu′GGCCCu′GCGGCAGGGCGGC | | |
| | Cu′GGu′GAu′GCAGAACu′ACCGGCu′GAu′CGACGCCAC | | |
| | CCu′Gu′ACGu′GACCu′u′CGAGCCCu′GCGu′GAu′Gu′GCG | | |
| | CCGGCGCCAu′GAu′CCACAGCCGGAu′CGGCCGGGu′GG | | |
| | u′Gu′u′CGGCGu′GCGGAACGCCAAGACCGGCGCCGCCG | | |
| | GCAGCCu′GAu′GGACGu′GCu′GCACCACCCCGGCAu′G | | |
| | AACCACCGGGu′GGAGAu′CACCGAGGGCAu′CCu′GGCC | | |
| | GACGAGu′GCGCCGCCCu′GCu′Gu′GCCGGu′u′Cu′u′CCG | | |
| | GAu′GCCCCGGCGGGu′Gu′u′CAACGCCCAGAAGAAGG | | |
| | CCCAGAGCAGCACCGACAGCGGCGGCAGCAGCGGCG | | |
| | GCAGCAGCGGCAGCGAGACACCCGGCACCAGCGAGA | | |
| | GCGCCACCCCCGAGAGCAGCGGCGGCAGCAGCGGCG | | |
| | GCAGCGACAAGAAGu′ACAGCAu′CGGCCu′GGCCAu′C | | |
| | GGCACCAACAGCGu′GGGCu′GGGCCGu′GAu′CACCGAC | | |
| | GAGu′ACAAGGu′GCCCAGCAAGAAGu′u′CAAGGu′GCu′ | | |
| | GGGCAACACCGACCGGCACAGCAu′CAAGAAGAACCU′ | | |
| | GAu′CGGCGCCCu′GCu′Gu′u′CGACAGCGGCGAGACAG | | |
| | CCGAGGCCACCCGGCu′GAAGCGGACCGCCCGGCGGC | | |
| | GGu′ACACCCGGCGGAAGAACCGGAu′Cu′GCu′ACCu′G | | |
| | CAGGAGAu′Cu′u′CAGCAACGAGAu′GGCCAAGGu′GGA | | |
| | CGACAGCu′u′Cu′u′CCACCGGCu′GGAGGAGAGCu′u′CC | | |
| | u′GGu′GGAGGAGGACAAGAAGCACGAGCGGCACCCCA | | |
| | u′Cu′u′CGGCAACAu′CGu′GGACGAGGu′GGCCu′ACCAC | | |
| | GAGAAGu′ACCCCACCAu′Cu′ACCACCu′GCGGAAGAAG | | |
| | Cu′GGu′GGACAGCACCGACAAGGCCGACCu′GCGGCu′G | | |
| | Au′Cu′ACCu′GGCCCu′GGCCCACAu′GAu′CAAGu′u′CCG | | |
| | GGGCCACu′u′CCu′GAu′CGAGGGCGACCu′GAACCCCG | | |
| | ACAACAGCGACGu′GGACAAGCu′Gu′u′CAu′CCAGCu′G | | |
| | Gu′GCAGACCu′ACAACCAGCu′Gu′u′CGAGGAGAACCC | | |
| | CAu′CAACGCCAGCGGCGu′GGACGCCAAGGCCAu′CCu′ | | |
| | GAGCGCCCGGCu′GAGCAAGAGCCGGCGGCu′GGAGAA | | |
| | CCu′GAu′CGCCCAGCu′GCCCGGCGAGAAGAAGAACGG | | |
| | CCu′Gu′u′CGGCAACCu′GAu′CGCCCu′GAGCCu′GGGCCu | | |
| | ′GACCCCCAACu′u′CAAGAGCAACu′u′CGACCu′GGCCG | | |
| | AGGACGCCAAGCu′GCAGCu′GAGCAAGGACACCu′ACG | | |
| | ACGACGACCu′GGACAACCu′GCu′GGCCCAGAu′CGGCG | | |
| | ACCAGu′ACGCCGACCu′Gu′u′CCu′GGCCGCCAAGAACC | | |
| | u′GAGCGACGCCAu′CCu′GCu′GAGCGACAu′CCu′GCGG | | |
| | Gu′GAACACCGAGAu′CACCAAGGCCCCCCu′GAGCGCC | | |
| | AGCAu′GAu′CAAGCGGu′ACGACGAGCACCACCAGGAC | | |
| | Cu′GACCCu′GCu′GAAGGCCCu′GGu′GCGGCAGCAGCu′ | | |
| | GCCCGAGAAGu′ACAAGGAGAu′Cu′u′Cu′u′CGACCAGA | | |
| | GCAAGAACGGCu′ACGCCGGCu′ACAu′CGACGGCGGCG | | |
| | CCAGCCAGGAGGAGu′u′Cu′ACAAGu′u′CAu′CAAGCCC | | |
| | Au′CCu′GGAGAAGAu′GGACGGCACCGAGGAGCu′GCu′ | | |
| | GGu′GAAGCu′GAACCGGGAGGACCu′GCu′GCGGAAGC | | |
| | AGCGGACCu′u′CGACAACGGCAGCAu′CCCCCACCAGA | | |
| | u′CCACCu′GGGCGAGCu′GCACGCCAu′CCu′GCGGCGGC | | |
| | AGGAGGACu′u′Cu′ACCCCu′u′CCu′GAAGGACAACCGG | | |
| | GAGAAGAu′CGAGAAGAu′CCu′GACCu′u′CCGGAu′CCC | | |
| | Cu′ACu′ACGu′GGGCCCCCu′GGCCCGGGGCAACAGCCG | | |
| | Gu′u′CGCCu′GGAu′GACCCGCAAGAGCGAGGAGACAAu | | |
| | ′CACCCCCu′GGAACu′u′CGAGGAGGu′GGu′GGACAAGG | | |
| | GCGCCAGCGCCCAGAGCu′u′CAu′CGAGCGGAu′GACCA | | |
| | ACu′u′CGACAAGAACCu′GCCCAACGAGAAGGu′GCu′G | | |
| | CCCAAGCACAGCCu′GCu′Gu′ACGAGu′ACu′u′CACCGu′ | | |
| | Gu′ACAACGAGCu′GACCAAGGu′GAAGu′ACGu′GACCG | | |
| | AGGGCAu′GCGGAAGCCCGCCu′u′CCu′GAGCGGCGAGC | | |
| | AGAAGAAGGCCAu′CGu′GGACCu′GCu′Gu′u′CAAGACC | | |
| | AACCGGAAGGu′GACCGu′GAAGCAGCu′GAAGGAGGA | | |
| | Cu′ACu′u′CAAGAAGAu′CGAGu′GCu′u′CGACAGCGu′GG | | |
| | AGAu′CAGCGGCGu′GGAGGACCGGu′u′CAACGCCAGC | | |
| | Cu′GGGCACCu′ACCACGACCu′GCu′GAAGAu′CAu′CAA | | |
| | GGACAAGGACu′u′CCu′GGACAACGAGGAGAACGAGG | | |
| | ACAu′CCu′GGAGGACAu′CGu′GCu′GACCCu′GACCCu′G | | |
| | u′u′CGAGGACCGGGAGAu′GAu′CGAGGAGCGGCu′GAA | | |
| | GACCu′ACGCCCACCu′Gu′u′CGACGACAAGGu′GAu′GA | | |
| | AGCAGCu′GAAGCGGCGGCGGu′ACACCGGCu′GGGGCC | | |
| | GGCu′GAGCCGGAAGCu′GAu′CAACGGCAu′CCGGGAC | | |
| | AAGCAGAGCGGCAAGACCAu′CCu′GGACu′u′CCu′CAA | | |
| | GAGCGACGGCu′u′CGCCAACCGGAACu′u′CAu′GCAGC | | |
| | u′GAu′CCACGACGACAGCCu′GACCu′u′CAAGGAGGAC | | |
| | Au′CCAGAAGGCCCAGGu′GAGCGGCCAGGGCGACAGC | | |
| | Cu′GCACGAGCACAu′CGCCAACCu′GGCCGGCAGCCCC | | |
| | GCCAu′CAAGAAGGGCAu′CCu′GCAGACCGu′GAAGGu′ | | |
| | GGu′GGACGAGCu′GGu′GAAGGu′GAu′GGGCCGGCACA | | |
| | AGCCCGAGAACAu′CGu′GAu′CGAGAu′GGCCCGGGAG | | |
| | AACCAGACCACCCAGAAGGGCCAGAAGAACAGCCGG | | |
| | GAGCGGAu′GAAGCGGAu′CGAGGAGGGCAu′CAAGGA | | |
| | GCu′GGGCAGCCAGAu′CCu′GAAGGAGCACCCCGu′GG | | |
| | AGAACACCCAGCu′GCAGAACGAGAAGCu′Gu′ACCu′Gu | | |
| | ′ACu′ACCu′GCAGAACGGCCGGGACAu′Gu′ACGu′GGAC | | |
| | CAGGAGCu′GGACAu′CAACCGGCu′GAGCGACu′ACGA | | |
| | CGu′GGACCACAu′CGu′GCCCCAGAGCu′u′CCu′GAAGG | | |
| | ACGACAGCAu′CGACAACAAGGu′GCu′GACCCGGAGCG | | |
| | ACAAGAACCGGGGCAAGAGCGACAACGu′GCCCAGCG | | |
| | AGGAGGu′GGu′GAAGAAGAu′GAAGAACu′ACu′GGCGG | | |
| | CAGCu′GCu′GAACGCCAAGCu′GAu′CACCCAGCGGAAG | | |
| | u′u′CGACAACCu′GACCAAGGCCGAGCGGGGCGGCCu′G | | |
| | AGCGAGCu′GGACAAGGCCGGCu′u′CAu′CAAGCGGCA | | |
| | GCu′GGu′GGAGACACGGCAGAu′CACCAAGCACGu′GG | | |
| | CCCAGAu′CCu′GGACAGCCGGAu′GAACACCAAGu′ACG | | |
| | ACGAGAACGACAAGCu′GAu′CCGGGAGGu′GAAGGu′G | | |
| | Au′CACCCu′CAAGAGCAAGCu′GGu′GAGCGACu′u′CCG | | |
| | GAAGGACu′u′CCAGu′u′Cu′ACAAGGu′GCGGGAGAu′CA | | |
| | ACAACu′ACCACCACGCCCACGACGCCu′ACCu′GAACG | | |
| | CCGu′GGu′GGGCACCGCCCu′GAu′CAAGAAGu′ACCCC | | |
| | AAGCu′GGAGAGCGAGu′u′CGu′Gu′ACGGCGACu′ACAA | | |
| | GGu′Gu′ACGACGu′GCGGAAGAu′GAu′CGCCAAGAGCG | | |
| | AGCAGGAGAu′CGGCAAGGCCACCGCCAAGu′ACu′u′Cu | | |
| | ′u′Cu′ACAGCAACAu′CAu′GAACu′u′Cu′u′CAAGACCGA | | |
| | GAu′CACCCu′GGCCAACGGCGAGAu′CCGGAAGCGGCC | | |
| | CCu′GAu′CGAGACAAACGGCGAGACAGGCGAGAu′CGu | | |
| | ′Gu′GGGACAAGGGCCGGGACu′u′CGCCACCGu′GCGGA | | |
| | AGGu′GCu′GAGCAu′GCCCCAGGu′GAACAu′CGu′GAAG | | |
| | AAGACCGAGGu′GCAGACCGGCGGCu′u′CAGCAAGGA | | |
| | GAGCAu′CCu′GCCCAAGCGGAACAGCGACAAGCu′GAu | | |
| | ′CGCCCGGAAGAAGGACu′GGGACCCCAAGAAGu′ACG | | |
| | GCGGCu′u′CGAGAGCCCCACCGu′GGCCu′ACAGCGu′G | | |
| | Cu′GGu′GGu′GGCCAAGGu′GGAGAAGGGCAAGAGCAA | | |
| | GAAGCu′CAAGAGCGu′GAAGGAGCu′GCu′GGGCAu′CA | | |
| | CCAu′CAu′GGAGCGGAGCAGCu′u′CGAGAAGAACCCC | | |
| | Au′CGACu′u′CCu′GGAGGCCAAGGGCu′ACAAGGAGGu′ | | |
| | GAAGAAGGACCu′GAu′CAu′CAAGCu′GCCCAAGu′ACA | | |
| | GCCu′Gu′u′CGAGCu′GGAGAACGGCCGGAAGCGGAu′G | | |
| | Cu′GGCCAGCGCCGGCGAGCu′GCAGAAGGGCAACGAG | | |
| | Cu′GGCCCu′GCCCAGCAAGu′ACGu′GAACu′u′CCu′Gu′A | | |
| | CCu′GGCCAGCCACu′ACGAGAAGCu′GAAGGGCAGCCC | | |
| | CGAGGACAACGAGCAGAAGCAGCu′Gu′u′CGu′GGAGC | | |
| | AGCACAAGCACu′ACCu′GGACGAGAu′CAu′CGAGCAG | | |
| | Au′CAGCGAGu′u′CAGCAAGCGGGu′GAu′CCu′GGCCGA | | |
| | CGCCAACCu′GGACAAGGu′GCu′GAGCGCCu′ACAACAA | | |
| | GCACCGGGACAAGCCCAu′CCGGGAGCAGGCCGAGAA | | |
| | CAu′CAu′CCACCu′Gu′u′CACCCu′GACCAACCu′GGGCG | | |
| | CCCCCGCCGCCu′u′CAAGu′ACu′u′CGACACCACCAu′CG | | |
| | ACCGGAAGCGGu′ACACCAGCACCAAGGAGGu′GCu′GG | | |
| | ACGCCACCCu′GAu′CCACCAGAGCAu′CACCGGCCu′Gu′ | | |
| | ACGAGACACGGAu′CGACCu′GAGCCAGCu′GGGCGGCG | | |
| | ACGAGGGCGCCGACAAGCGGACCGCCGACGGCAGCG | | |
| | AGu′u′CGAGAGCCCCAAGAAGAAGCGGAAGGu′Gu′GA | | |
| | GCGGCCGCu′u′AAu′u′AAGCu′GCCu′u′Cu′GCGGGGCu′u | | |
| | ′GCCu′u′Cu′GGCCAu′GCCCu′u′Cu′u′Cu′Cu′CCCu′u′GCA | | |
| | CCu′Gu′ACCu′Cu′u′GGu′Cu′u′u′GAAu′AAAGCCu′GAGu′ | | |
| | AGGAAGu′Cu′AGA | | |
| | MSEVEFSHEYWMRHALTLAKRARDEREVPVGAVLVLN | Protein | 688 |
| | NRVIGEGWNRAIGLHDPTAHAEIMALRQGGLVMQNYRL | | |
| | IDATLYVTFEPCVMCAGAMIHSRIGRVVFGVRNAKTGA | | |
| | AGSLMDVLHHPGMNHRVEITEGILADECAALLCRFFRMP | | |
| | RRVFNAQKKAQSSTDSGGSSGGSSGSETPGTSESATPESS | | |
| | GGSSGGSDKKYSIGLAIGTNSVGWAVITDEYKVPSKKFK | | |
| | VLGNTDRHSIKKNLIGALLFDSGETAEATRLKRTARRRY | | |
| | TRRKNRICYLQEIFSNEMAKVDDSFFHRLEESFLVEEDKK | | |
| | HERHPIFGNIVDEVAYHEKYPTIYHLRKKLVDSTDKADL | | |
| | RLIYLALAHMIKFRGHFLIEGDLNPDNSDVDKLFIQLVQT | | |
| | YNQLFEENPINASGVDAKAILSARLSKSRRLENLIAQLPG | | |
| | EKKNGLFGNLIALSLGLTPNFKSNFDLAEDAKLQLSKDT | | |
| | YDDDLDNLLAQIGDQYADLFLAAKNLSDAILLSDILRVN | | |
| | TEITKAPLSASMIKRYDEHHQDLTLLKALVRQQLPEKYK | | |
| | EIFFDQSKNGYAGYIDGGASQEEFYKFIKPILEKMDGTEE | | |
| | LLVKLNREDLLRKQRTFDNGSIPHQIHLGELHAILRRQED | | |
| | FYPFLKDNREKIEKILTFRIPYYVGPLARGNSRFAWMTRK | | |
| | SEETITPWNFEEVVDKGASAQSFIERMTNFDKNLPNEKV | | |
| | LPKHSLLYEYFTVYNELTKVKYVTEGMRKPAFLSGEQK | | |
| | KAIVDLLFKTNRKVTVKQLKEDYFKKIECFDSVEISGVED | | |
| | RFNASLGTYHDLLKIIKDKDFLDNEENEDILEDIVLTLTLF | | |
| | EDREMIEERLKTYAHLFDDKVMKQLKRRRYTGWGRLSR | | |
| | KLINGIRDKQSGKTILDFLKSDGFANRNFMQLIHDDSLTF | | |
| | KEDIQKAQVSGQGDSLHEHIANLAGSPAIKKGILQTVKV | | |
| | VDELVKVMGRHKPENIVIEMARENQTTQKGQKNSRERM | | |
| | KRIEEGIKELGSQILKEHPVENTQLQNEKLYLYYLQNGR | | |
| | DMYVDQELDINRLSDYDVDHIVPQSFLKDDSIDNKVLTR | | |
| | SDKNRGKSDNVPSEEVVKKMKNYWRQLLNAKLITQRKF | | |
| | DNLTKAERGGLSELDKAGFIKRQLVETRQITKHVAQILD | | |
| | SRMNTKYDENDKLIREVKVITLKSKLVSDFRKDFQFYKV | | |
| | REINNYHHAHDAYLNAVVGTALIKKYPKLESEFVYGDY | | |
| | KVYDVRKMIAKSEQEIGKATAKYFFYSNIMNFFKTEITL | | |
| | ANGEIRKRPLIETNGETGEIVWDKGRDFATVRKVLSMPQ | | |
| | VNIVKKTEVQTGGFSKESILPKRNSDKLIARKKDWDPKK | | |
| | YGGFESPTVAYSVLVVAKVEKGKSKKLKSVKELLGITIM | | |
| | ERSSFEKNPIDFLEAKGYKEVKKDLIIKLPKYSLFELENGR | | |
| | KRMLASAGELQKGNELALPSKYVNFLYLASHYEKLKGS | | |
| | PEDNEQKQLFVEQHKHYLDEIIEQISEFSKRVILADANLD | | |
| | KVLSAYNKHRDKPIREQAENIIHLFTLTNLGAPAAFKYFD | | |
| | TTIDRKRYTSTKEVLDATLIHQSITGLYETRIDLSQLGGDE | | |
| | GADKRTADGSEFESPKKKRKV | | |
| 5′ UTR | AGGAAAu′AAGAGAGAAAAGAAGAGu′AAGAAGAAAu′ | mRNA | 689 |
| | Au′AAGAGCCACC | | |
| TadA | Au′GAGCGAGGu′GGAGu′u′CAGCCACGAGu′ACu′GGAu′ | mRNA | 690 |
| | GCGGCACGCCCu′GACCCu′GGCCAAGCGGGCCCGGGA | | |
| | CGAGCGGGAGGu′GCCCGu′GGGCGCCGu′GCu′GGu′GCu | | |
| | ′GAACAACCGGGu′GAu′CGGCGAGGGCu′GGAACCGGG | | |
| | CCAu′CGGCCu′GCACGACCCCACCGCCCACGCCGAGAu | | |
| | ′CAu′GGCCCu′GCGGCAGGGCGGCCu′GGu′GAu′GCAGA | | |
| | ACu′ACCGGCu′GAu′CGACGCCACCCu′Gu′ACGu′GACCu | | |
| | ′u′CGAGCCCu′GCGu′GAu′Gu′GCGCCGGCGCCAu′GAu′C | | |
| | CACAGCCGGAu′CGGCCGGGu′GGu′Gu′u′CGGCGu′GCG | | |
| | GAACGCCAAGACCGGCGCCGCCGGCAGCCu′GAu′GGA | | |
| | CGu′GCu′GCACCACCCCGGCAu′GAACCACCGGGu′GGA | | |
| | GAu′CACCGAGGGCAu′CCu′GGCCGACGAGu′GCGCCGC | | |
| | CCu′GCu′Gu′GCCGGu′u′Cu′u′CCGGAu′GCCCCGGCGGG | | |
| | u′Gu′u′CAACGCCCAGAAGAAGGCCCAGAGCAGCACCG | | |
| | AC | | |
| | MSEVEFSHEYWMRHALTLAKRARDEREVPVGAVLVLN | Protein | 691 |
| | NRVIGEGWNRAIGLHDPTAHAEIMALRQGGLVMQNYRL | | |
| | IDATLYVTFEPCVMCAGAMIHSRIGRVVFGVRNAKTGA | | |
| | AGSLMDVLHHPGMNHRVEITEGILADECAALLCRFFRMP | | |
| | RRVFNAQKKAQSSTD | | |
| Linker | AGCGGCGGCAGCAGCGGCGGCAGCAGCGGCAGCGAG | mRNA | 692 |
| between | ACACCCGGCACCAGCGAGAGCGCCACCCCCGAGAGCA | | |
| TadA | GCGGCGGCAGCAGCGGCGGCAGC | | |
| and Cas9 | SGGSSGGSSGSETPGTSESATPESSGGSSGGS | Protein | 693 |
| nickase | | | |
| Cas9 | GACAAGAAGu′ACAGCAu′CGGCCu′GGCCAu′CGGCACC | mRNA | 694 |
| nickase | AACAGCGu′GGGCu′GGGCCGu′GAu′CACCGACGAGu′A | | |
| | CAAGGu′GCCCAGCAAGAAGu′u′CAAGGu′GCu′GGGCA | | |
| | ACACCGACCGGCACAGCAu′CAAGAAGAACCu′GAu′CG | | |
| | GCGCCCu′GCu′Gu′u′CGACAGCGGCGAGACAGCCGAGG | | |
| | CCACCCGGCu′GAAGCGGACCGCCCGGCGGCGGu′ACA | | |
| | CCCGGCGGAAGAACCGGAu′Cu′GCu′ACCu′GCAGGAG | | |
| | Au′Cu′u′CAGCAACGAGAu′GGCCAAGGu′GGACGACAG | | |
| | Cu′u′Cu′u′CCACCGGCu′GGAGGAGAGCu′u′CCu′GGu′GG | | |
| | AGGAGGACAAGAAGCACGAGCGGCACCCCAu′Cu′u′CG | | |
| | GCAACAu′CGu′GGACGAGGu′GGCCu′ACCACGAGAAGu | | |
| | ′ACCCCACCAu′Cu′ACCACCu′GCGGAAGAAGCu′GGu′G | | |
| | GACAGCACCGACAAGGCCGACCu′GCGGCu′GAu′Cu′AC | | |
| | Cu′GGCCCu′GGCCCACAu′GAu′CAAGu′u′CCGGGGCCA | | |
| | Cu′u′CCu′GAu′CGAGGGCGACCu′GAACCCCGACAACA | | |
| | GCGACGu′GGACAAGCu′Gu′u′CAu′CCAGCu′GGu′GCAG | | |
| | ACCu′ACAACCAGCu′Gu′u′CGAGGAGAACCCCAu′CAA | | |
| | CGCCAGCGGCGu′GGACGCCAAGGCCAu′CCu′GAGCGC | | |
| | CCGGCu′GAGCAAGAGCCGGCGGCu′GGAGAACCu′GAu | | |
| | ′CGCCCAGCu′GCCCGGCGAGAAGAAGAACGGCCu′Gu′u | | |
| | ′CGGCAACCu′GAu′CGCCCu′GAGCCu′GGGCCu′GACCC | | |
| | CCAACu′u′CAAGAGCAACu′u′CGACCu′GGCCGAGGAC | | |
| | GCCAAGCu′GCAGCu′GAGCAAGGACACCu′ACGACGAC | | |
| | GACCu′GGACAACCu′GCu′GGCCCAGAu′CGGCGACCAG | | |
| | u′ACGCCGACCu′Gu′u′CCu′GGCCGCCAAGAACCU′GAG | | |
| | CGACGCCAu′CCu′GCu′GAGCGACAu′CCu′GCGGGu′GA | | |
| | ACACCGAGAu′CACCAAGGCCCCCCu′GAGCGCCAGCA | | |
| | u′GAu′CAAGCGGu′ACGACGAGCACCACCAGGACCu′G | | |
| | ACCCu′GCu′GAAGGCCCu′GGu′GCGGCAGCAGCu′GCCC | | |
| | GAGAAGu′ACAAGGAGAu′Cu′u′Cu′u′CGACCAGAGCAA | | |
| | GAACGGCu′ACGCCGGCu′ACAu′CGACGGCGGCGCCAG | | |
| | CCAGGAGGAGu′u′Cu′ACAAGu′u′CAu′CAAGCCCAu′CC | | |
| | u′GGAGAAGAu′GGACGGCACCGAGGAGCu′GCu′GGu′G | | |
| | AAGCu′GAACCGGGAGGACCu′GCu′GCGGAAGCAGCG | | |
| | GACCu′u′CGACAACGGCAGCAu′CCCCCACCAGAu′CCA | | |
| | CCu′GGGCGAGCu′GCACGCCAu′CCu′GCGGCGGCAGGA | | |
| | GGACu′u′Cu′ACCCCu′u′CCu′GAAGGACAACCGGGAGA | | |
| | AGAu′CGAGAAGAu′CCu′GACCu′u′CCGGAu′CCCCu′AC | | |
| | u′ACGu′GGGCCCCCu′GGCCCGGGGCAACAGCCGGu′u′C | | |
| | GCCu′GGAu′GACCCGCAAGAGCGAGGAGACAAu′CACC | | |
| | CCCu′GGAACu′u′CGAGGAGGu′GGu′GGACAAGGGCGC | | |
| | CAGCGCCCAGAGCu′u′CAu′CGAGCGGAu′GACCAACu′u | | |
| | ′CGACAAGAACCu′GCCCAACGAGAAGGu′GCu′GCCCA | | |
| | AGCACAGCCu′GCu′Gu′ACGAGu′ACu′u′CACCGu′Gu′AC | | |
| | AACGAGCu′GACCAAGGu′GAAGu′ACGu′GACCGAGGG | | |
| | CAu′GCGGAAGCCCGCCu′u′CCu′GAGCGGCGAGCAGAA | | |
| | GAAGGCCAu′CGu′GGACCu′GCu′Gu′u′CAAGACCAACC | | |
| | GGAAGGu′GACCGu′GAAGCAGCu′GAAGGAGGACu′AC | | |
| | u′u′CAAGAAGAu′CGAGu′GCu′u′CGACAGCGu′GGAGAu | | |
| | ′CAGCGGCGu′GGAGGACCGGu′u′CAACGCCAGCCu′GG | | |
| | GCACCu′ACCACGACCu′GCu′GAAGAu′CAu′CAAGGAC | | |
| | AAGGACu′u′CCu′GGACAACGAGGAGAACGAGGACAu′ | | |
| | CCu′GGAGGACAu′CGu′GCu′GACCCu′GACCCu′Gu′u′CG | | |
| | AGGACCGGGAGAu′GAu′CGAGGAGCGGCu′GAAGACCu | | |
| | ′ACGCCCACCu′Gu′u′CGACGACAAGGu′GAu′GAAGCAG | | |
| | Cu′GAAGCGGCGGCGGu′ACACCGGCu′GGGGCCGGCu′ | | |
| | GAGCCGGAAGCu′GAu′CAACGGCAu′CCGGGACAAGCA | | |
| | GAGCGGCAAGACCAu′CCu′GGACu′u′CCu′CAAGAGCG | | |
| | ACGGCu′u′CGCCAACCGGAACu′u′CAu′GCAGCu′GAu′C | | |
| | CACGACGACAGCCu′GACCu′u′CAAGGAGGACAu′CCAG | | |
| | AAGGCCCAGGu′GAGCGGCCAGGGCGACAGCCu′GCAC | | |
| | GAGCACAu′CGCCAACCu′GGCCGGCAGCCCCGCCAu′C | | |
| | AAGAAGGGCAu′CCu′GCAGACCGu′GAAGGu′GGu′GGA | | |
| | CGAGCu′GGu′GAAGGu′GAu′GGGCCGGCACAAGCCCG | | |
| | AGAACAu′CGu′GAu′CGAGAu′GGCCCGGGAGAACCAG | | |
| | ACCACCCAGAAGGGCCAGAAGAACAGCCGGGAGCGG | | |
| | Au′GAAGCGGAu′CGAGGAGGGCAu′CAAGGAGCu′GGG | | |
| | CAGCCAGAu′CCu′GAAGGAGCACCCCGu′GGAGAACAC | | |
| | CCAGCu′GCAGAACGAGAAGCu′Gu′ACCu′Gu′ACu′ACC | | |
| | u′GCAGAACGGCCGGGACAu′Gu′ACGu′GGACCAGGAG | | |
| | Cu′GGACAu′CAACCGGCu′GAGCGACu′ACGACGu′GGA | | |
| | CCACAu′CGu′GCCCCAGAGCu′u′CCu′GAAGGACGACA | | |
| | GCAu′CGACAACAAGGu′GCu′GACCCGGAGCGACAAGA | | |
| | ACCGGGGCAAGAGCGACAACGu′GCCCAGCGAGGAGG | | |
| | u′GGu′GAAGAAGAu′GAAGAACu′ACu′GGCGGCAGCu′G | | |
| | Cu′GAACGCCAAGCu′GAu′CACCCAGCGGAAGu′u′CGA | | |
| | CAACCu′GACCAAGGCCGAGCGGGGCGGCCu′GAGCGA | | |
| | GCu′GGACAAGGCCGGCu′u′CAu′CAAGCGGCAGCu′GGu | | |
| | ′GGAGACACGGCAGAu′CACCAAGCACGu′GGCCCAGAu | | |
| | ′CCu′GGACAGCCGGAu′GAACACCAAGu′ACGACGAGA | | |
| | ACGACAAGCu′GAu′CCGGGAGGu′GAAGGu′GAu′CACC | | |
| | Cu′CAAGAGCAAGCu′GGu′GAGCGACu′u′CCGGAAGGA | | |
| | Cu′u′CCAGu′u′Cu′ACAAGGu′GCGGGAGAu′CAACAACu′ | | |
| | ACCACCACGCCCACGACGCCu′ACCu′GAACGCCGu′GG | | |
| | u′GGGCACCGCCCu′GAu′CAAGAAGu′ACCCCAAGCu′G | | |
| | GAGAGCGAGu′u′CGu′Gu′ACGGCGACu′ACAAGGu′Gu′A | | |
| | CGACGu′GCGGAAGAu′GAu′CGCCAAGAGCGAGCAGG | | |
| | AGAu′CGGCAAGGCCACCGCCAAGu′ACu′u′Cu′u′Cu′AC | | |
| | AGCAACAu′CAu′GAACu′u′Cu′u′CAAGACCGAGAu′CAC | | |
| | CCu′GGCCAACGGCGAGAu′CCGGAAGCGGCCCCu′GAu′ | | |
| | CGAGACAAACGGCGAGACAGGCGAGAu′CGu′Gu′GGG | | |
| | ACAAGGGCCGGGACu′u′CGCCACCGu′GCGGAAGGu′G | | |
| | Cu′GAGCAu′GCCCCAGGu′GAACAu′CGu′GAAGAAGAC | | |
| | CGAGGu′GCAGACCGGCGGCu′u′CAGCAAGGAGAGCAu | | |
| | ′CCu′GCCCAAGCGGAACAGCGACAAGCu′GAu′CGCCC | | |
| | GGAAGAAGGACu′GGGACCCCAAGAAGu′ACGGCGGCu | | |
| | ′u′CGAGAGCCCCACCGu′GGCCu′ACAGCGu′GCu′GGu′G | | |
| | Gu′GGCCAAGGu′GGAGAAGGGCAAGAGCAAGAAGCu′ | | |
| | CAAGAGCGu′GAAGGAGCu′GCu′GGGCAu′CACCAu′CA | | |
| | u′GGAGCGGAGCAGCu′u′CGAGAAGAACCCCAu′CGACu | | |
| | ′u′CCu′GGAGGCCAAGGGCu′ACAAGGAGGu′GAAGAAG | | |
| | GACCu′GAu′CAu′CAAGCu′GCCCAAGu′ACAGCCu′Gu′u′ | | |
| | CGAGCu′GGAGAACGGCCGGAAGCGGAu′GCu′GGCCA | | |
| | GCGCCGGCGAGCu′GCAGAAGGGCAACGAGCu′GGCCC | | |
| | u′GCCCAGCAAGu′ACGu′GAACu′u′CCu′Gu′ACCu′GGCC | | |
| | AGCCACu′ACGAGAAGCu′GAAGGGCAGCCCCGAGGAC | | |
| | AACGAGCAGAAGCAGCu′Gu′u′CGu′GGAGCAGCACAA | | |
| | GCACu′ACCu′GGACGAGAu′CAu′CGAGCAGAu′CAGCG | | |
| | AGu′u′CAGCAAGCGGGu′GAu′CCu′GGCCGACGCCAAC | | |
| | Cu′GGACAAGGu′GCu′GAGCGCCu′ACAACAAGCACCG | | |
| | GGACAAGCCCAu′CCGGGAGCAGGCCGAGAACAu′CAu′ | | |
| | CCACCu′Gu′u′CACCCu′GACCAACCu′GGGCGCCCCCGC | | |
| | CGCCu′u′CAAGu′ACu′u′CGACACCACCAu′CGACCGGA | | |
| | AGCGGu′ACACCAGCACCAAGGAGGu′GCu′GGACGCCA | | |
| | CCCu′GAu′CCACCAGAGCAu′CACCGGCCu′Gu′ACGAGA | | |
| | CACGGAu′CGACCu′GAGCCAGCu′GGGCGGCGAC | | |
| | DKKYSIGLAIGTNSVGWAVITDEYKVPSKKFKVLGNTDR | Protein | 695 |
| | HSIKKNLIGALLFDSGETAEATRLKRTARRRYTRRKNRIC | | |
| | YLQEIFSNEMAKVDDSFFHRLEESFLVEEDKKHERHPIFG | | |
| | NIVDEVAYHEKYPTIYHLRKKLVDSTDKADLRLIYLALA | | |
| | HMIKFRGHFLIEGDLNPDNSDVDKLFIQLVQTYNQLFEE | | |
| | NPINASGVDAKAILSARLSKSRRLENLIAQLPGEKKNGLF | | |
| | GNLIALSLGLTPNFKSNFDLAEDAKLQLSKDTYDDDLDN | | |
| | LLAQIGDQYADLFLAAKNLSDAILLSDILRVNTEITKAPL | | |
| | SASMIKRYDEHHQDLTLLKALVRQQLPEKYKEIFFDQSK | | |
| | NGYAGYIDGGASQEEFYKFIKPILEKMDGTEELLVKLNR | | |
| | EDLLRKQRTFDNGSIPHQIHLGELHAILRRQEDFYPFLKD | | |
| | NREKIEKILTFRIPYYVGPLARGNSRFAWMTRKSEETITP | | |
| | WNFEEVVDKGASAQSFIERMTNFDKNLPNEKVLPKHSLL | | |
| | YEYFTVYNELTKVKYVTEGMRKPAFLSGEQKKAIVDLL | | |
| | FKTNRKVTVKQLKEDYFKKIECFDSVEISGVEDRFNASL | | |
| | GTYHDLLKIIKDKDFLDNEENEDILEDIVLTLTLFEDREMI | | |
| | EERLKTYAHLFDDKVMKQLKRRRYTGWGRLSRKLINGI | | |
| | RDKQSGKTILDFLKSDGFANRNFMQLIHDDSLTFKEDIQ | | |
| | KAQVSGQGDSLHEHIANLAGSPAIKKGILQTVKVVDELV | | |
| | KVMGRHKPENIVIEMARENQTTQKGQKNSRERMKRIEE | | |
| | GIKELGSQILKEHPVENTQLQNEKLYLYYLQNGRDMYV | | |
| | DQELDINRLSDYDVDHIVPQSFLKDDSIDNKVLTRSDKN | | |
| | RGKSDNVPSEEVVKKMKNYWRQLLNAKLITQRKFDNLT | | |
| | KAERGGLSELDKAGFIKRQLVETRQITKHVAQILDSRMN | | |
| | TKYDENDKLIREVKVITLKSKLVSDFRKDFQFYKVREIN | | |
| | NYHHAHDAYLNAVVGTALIKKYPKLESEFVYGDYKVY | | |
| | DVRKMIAKSEQEIGKATAKYFFYSNIMNFFKTEITLANGE | | |
| | IRKRPLIETNGETGEIVWDKGRDFATVRKVLSMPQVNIV | | |
| | KKTEVQTGGFSKESILPKRNSDKLIARKKDWDPKKYGGF | | |
| | ESPTVAYSVLVVAKVEKGKSKKLKSVKELLGITIMERSS | | |
| | FEKNPIDFLEAKGYKEVKKDLIIKLPKYSLFELENGRKRM | | |
| | LASAGELQKGNELALPSKYVNFLYLASHYEKLKGSPEDN | | |
| | EQKQLFVEQHKHYLDEIIEQISEFSKRVILADANLDKVLS | | |
| | AYNKHRDKPIREQAENIIHLFTLTNLGAPAAFKYFDTTID | | |
| | RKRYTSTKEVLDATLIHQSITGLYETRIDLSQLGGD | | |
| Linker | GAGGGCGCCGAC | mRNA | 696 |
| between | | | |
| Cas9 | | | |
| nickase | EGAD | Protein | 697 |
| and NLS | | | |
| Nuclear | AAGCGGACCGCCGACGGCAGCGAGu′u′CGAGAGCCCC | mRNA | 698 |
| local- | AAGAAGAAGCGGAAGGu′Gu′GA | | |
| ization | KRTADGSEFESPKKKRKV | Protein | 699 |
| sequence | | | |
| (NLS) | | | |
| 3′ UTR | GCGGCCGCu′u′AAu′u′AAGCu′GCCu′u′Cu′GCGGGGCu′u | mRNA | 700 |
| | ′GCCu′u′Cu′GGCCAu′GCCCu′u′Cu′u′Cu′Cu′CCCu′u′GCA | | |
| | CCu′Gu′ACCu′Cu′u′GGu′Cu′u′u′GAAu′AAAGCCu′GAGu′ | | |
| | AGGAAGu′Cu′AGA | | |

MA079

| | | | |
|---|---|---|---|
| MA079 | AGGACu′CCu′CCCCAu′CCu′Cu′CCCu′Cu′Gu′CCCu′Cu′G | mRNA | 701 |
| | u′CCCu′Cu′GACCCu′GCACu′Gu′CCCAGCACCAu′GAGC | | |
| | GAGGu′GGAGu′u′CAGCCACGAGu′ACu′GGAu′GCGCCA | | |
| | CGCCCu′GACCCu′GGCCAAGCGCGCCCGCGACGAGCG | | |
| | CGAGGu′GCCCGu′GGGCGCCGu′GCu′GGu′GCu′GAACA | | |
| | ACCGCGu′GAu′CGGCGAGGGCu′GGAACCGCGCCAu′CG | | |
| | GCCu′GCACGACCCCACCGCCCACGCCGAGAu′CAu′GG | | |
| | CCCu′GCGCCAGGGCGGCCu′GGu′GAu′GCAGAACu′AC | | |
| | CGCCu′GAu′CGACGCCACCCu′Gu′ACGu′GACCu′u′CGA | | |
| | GCCCu′GCGu′GAu′Gu′GCGCCGGCGCCAu′GAu′CCACA | | |
| | GCCGCAu′CGGCCGCGu′GGu′Gu′u′CGGCGu′GCGCAAC | | |
| | GCCAAGACCGGCGCCGCCGGCAGCCu′GAu′GGACGu′G | | |
| | Cu′GCACCACCCCGGCAu′GAACCACCGCGu′GGAGAu′C | | |
| | ACCGAGGGCAu′CCu′GGCCGACGAGu′GCGCCGCCCu′G | | |
| | Cu′Gu′GCCGCu′u′Cu′u′CCGCAu′GCCCCGCCGCGu′Gu′u′ | | |
| | CAACGCCCAGAAGAAGGCCCAGAGCAGCACCGACAG | | |
| | CGGCGGCAGCAGCGGCGGCAGCAGCGGCAGCGAGAC | | |
| | ACCCGGCACCAGCGAGAGCGCCACCCCCGAGAGCAGC | | |
| | GGCGGCAGCAGCGGCGGCAGCGACAAGAAGu′ACAGC | | |
| | Au′CGGCCu′GGCCAu′CGGCACCAACAGCGu′GGGCu′G | | |
| | GGCCGu′GAu′CACCGACGAGu′ACAAGGu′GCCCAGCA | | |
| | AGAAGu′u′CAAGGu′GCu′GGGCAACACCGACCGCCAC | | |
| | AGCAu′CAAGAAGAACCu′GAu′CGGCGCCCu′GCu′Gu′u′ | | |
| | CGACAGCGGCGAGACAGCCGAGGCCACCCGCCu′GAA | | |
| | GCGCACCGCCCGCCGCCGCu′ACACCCGCCGCAAGAAC | | |
| | CGCAu′Cu′GCu′ACCu′GCAGGAGAu′Cu′u′CAGCAACGA | | |
| | GAu′GGCCAAGGu′GGACGACAGCu′u′Cu′u′CCACCGCCu | | |
| | ′GGAGGAGAGCu′u′CCu′GGu′GGAGGAGGACAAGAAGC | | |
| | ACGAGCGCCACCCCAu′Cu′u′CGGCAACAu′CGu′GGACG | | |
| | AGGu′GGCCu′ACCACGAGAAGu′ACCCCACCAu′Cu′ACC | | |
| | ACCu′GCGCAAGAAGCu′GGu′GGACAGCACCGACAAGG | | |
| | CCGACCu′GCGCCu′GAu′Cu′ACCu′GGCCCu′GGCCCACA | | |
| | u′GAu′CAAGu′u′CCGCGGCCACu′u′CCu′GAu′CGAGGGC | | |
| | GACCu′GAACCCCGACAACAGCGACGu′GGACAAGCu′G | | |
| | u′u′CAu′CCAGCu′GGu′GCAGACCu′ACAACCAGCu′Gu′u′ | | |
| | CGAGGAGAACCCCAu′CAACGCCAGCGGCGu′GGACGC | | |
| | CAAGGCCAu′CCu′GAGCGCCCGCCu′GAGCAAGAGCCG | | |
| | CCGCCu′GGAGAACCu′GAu′CGCCCAGCu′GCCCGGCGA | | |
| | GAAGAAGAACGGCCu′Gu′u′CGGCAACCu′GAu′CGCCCu | | |
| | ′GAGCCu′GGGCCu′GACCCCCAACu′u′CAAGAGCAACu′ | | |
| | u′CGACCu′GGCCGAGGACGCCAAGCu′GCAGCu′GAGCA | | |
| | AGGACACCu′ACGACGACGACCu′GGACAACCu′GCu′GG | | |
| | CCCAGAu′CGGCGACCAGu′ACGCCGACCu′Gu′u′CCu′G | | |
| | GCCGCCAAGAACCu′GAGCGACGCCAu′CCu′GCu′GAGC | | |
| | GACAu′CCu′GCGCGu′GAACACCGAGAu′CACCAAGGCC | | |
| | CCCCu′GAGCGCCAGCAu′GAu′CAAGCGCu′ACGACGAG | | |
| | CACCACCAGGACCu′GACCCu′GCu′GAAGGCCCu′GGu′G | | |
| | CGCCAGCAGCu′GCCCGAGAAGu′ACAAGGAGAu′Cu′u′ | | |
| | Cu′u′CGACCAGAGCAAGAACGGCu′ACGCCGGCu′ACAu | | |
| | ′CGACGGCGGCGCCAGCCAGGAGGAGu′u′Cu′ACAAGu′ | | |
| | u′CAu′CAAGCCCAu′CCu′GGAGAAGAu′GGACGGCACC | | |
| | GAGGAGCu′GCu′GGu′GAAGCu′GAACCGCGAGGACCu′ | | |
| | GCu′GCGCAAGCAGCGCACCu′u′CGACAACGGCAGCAu′ | | |
| | CCCCCACCAGAu′CCACCu′GGGCGAGCu′GCACGCCAu′ | | |
| | CCu′GCGCCGCCAGGAGGACu′u′Cu′ACCCCu′u′CCu′GA | | |
| | AGGACAACCGCGAGAAGAu′CGAGAAGAu′CCu′GACCu | | |
| | ′u′CCGCAu′CCCCu′ACu′ACGu′GGGCCCCCu′GGCCCGC | | |
| | GGCAACAGCCGCu′u′CGCCu′GGAu′GACCCGCAAGAGC | | |
| | GAGGAGACAAu′CACCCCCu′GGAACu′u′CGAGGAGGu′ | | |
| | GGu′GGACAAGGGCGCCAGCGCCCAGAGCu′u′CAu′CG | | |
| | AGCGCAu′GACCAACu′u′CGACAAGAACCu′GCCCAACG | | |
| | AGAAGGu′GCu′GCCCAAGCACAGCCu′GCu′Gu′ACGAGu | | |
| | ′ACu′u′CACCGu′Gu′ACAACGAGCu′GACCAAGGu′GAAG | | |
| | u′ACGu′GACCGAGGGCAu′GCGCAAGCCCGCCu′u′CCu′ | | |
| | GAGCGGCGAGCAGAAGAAGGCCAu′CGu′GGACCu′GCu | | |
| | ′Gu′u′CAAGACCAACCGCAAGGu′GACCGu′GAAGCAGC | | |
| | u′GAAGGAGGACu′ACu′u′CAAGAAGAu′CGAGu′GCu′u′ | | |
| | CGACAGCGu′GGAGAu′CAGCGGCGu′GGAGGACCGCu′u | | |
| | ′CAACGCCAGCCu′GGGCACCu′ACCACGACCu′GCu′GA | | |
| | AGAu′CAu′CAAGGACAAGGACu′u′CCu′GGACAACGAG | | |
| | GAGAACGAGGACAu′CCu′GGAGGACAu′CGu′GCu′GAC | | |
| | CCu′GACCCu′Gu′u′CGAGGACCGCGAGAu′GAu′CGAGG | | |
| | AGCGCCu′GAAGACCu′ACGCCCACCu′Gu′u′CGACGACA | | |
| | AGGu′GAu′GAAGCAGCu′GAAGCGCCGCCGCu′ACACC | | |
| | GGCu′GGGGCCGCCu′GAGCCGCAAGCu′GAu′CAACGGC | | |
| | Au′CCGCGACAAGCAGAGCGGCAAGACCAu′CCu′GGAC | | |
| | u′u′CCu′CAAGAGCGACGGCu′u′CGCCAACCGCAACu′u′ | | |
| | CAu′GCAGCu′GAu′CCACGACGACAGCCu′GACCu′u′CA | | |
| | AGGAGGACAu′CCAGAAGGCCCAGGu′GAGCGGCCAGG | | |
| | GCGACAGCCu′GCACGAGCACAu′CGCCAACCu′GGCCG | | |
| | GCAGCCCCGCCAu′CAAGAAGGGCAu′CCu′GCAGACCG | | |
| | u′GAAGGu′GGu′GGACGAGCu′GGu′GAAGGu′GAu′GGG | | |
| | CCGCCACAAGCCCGAGAACAu′CGu′GAu′CGAGAu′GGC | | |
| | CCGCGAGAACCAGACCACCCAGAAGGGCCAGAAGAA | | |
| | CAGCCGCGAGCGCAu′GAAGCGCAu′CGAGGAGGGCAu′ | | |
| | CAAGGAGCu′GGGCAGCCAGAu′CCu′GAAGGAGCACCC | | |
| | CGu′GGAGAACACCCAGCu′GCAGAACGAGAAGCu′Gu′ | | |
| | ACCu′Gu′ACu′ACCu′GCAGAACGGCCGCGACAu′Gu′AC | | |
| | Gu′GGACCAGGAGCu′GGACAu′CAACCGCCu′GAGCGA | | |
| | Cu′ACGACGu′GGACCACAu′CGu′GCCCCAGAGCu′u′CCu | | |
| | ′GAAGGACGACAGCAu′CGACAACAAGGu′GCu′GACCC | | |
| | GCAGCGACAAGAACCGCGGCAAGAGCGACAACGu′GC | | |
| | CCAGCGAGGAGGu′GGu′GAAGAAGAu′GAAGAACu′AC | | |
| | u′GGCGCCAGCu′GCu′GAACGCCAAGCu′GAu′CACCCA | | |
| | GCGCAAGu′u′CGACAACCu′GACCAAGGCCGAGCGCGG | | |
| | CGGCCu′GAGCGAGCu′GGACAAGGCCGGCu′u′CAu′CA | | |
| | AGCGCCAGCu′GGu′GGAGACACGCCAGAu′CACCAAGC | | |
| | ACGu′GGCCCAGAu′CCu′GGACAGCCGCAu′GAACACCA | | |
| | AGu′ACGACGAGAACGACAAGCu′GAu′CCGCGAGGu′G | | |
| | AAGGu′GAu′CACCCu′CAAGAGCAAGCu′GGu′GAGCGA | | |
| | Cu′u′CCGCAAGGACu′u′CCAGu′u′Cu′ACAAGGu′GCGCG | | |
| | AGAu′CAACAACu′ACCACCACGCCCACGACGCCu′ACC | | |
| | u′GAACGCCGu′GGu′GGGCACCGCCCu′GAu′CAAGAAGu | | |
| | ′ACCCCAAGCu′GGAGAGCGAGu′u′CGu′Gu′ACGGCGAC | | |
| | u′ACAAGGu′Gu′ACGACGu′GCGCAAGAu′GAu′CGCCAA | | |
| | GAGCGAGCAGGAGAu′CGGCAAGGCCACCGCCAAGu′A | | |
| | Cu′u′Cu′u′Cu′ACAGCAACAu′CAu′GAACu′u′Cu′u′CAAG | | |
| | ACCGAGAu′CACCCu′GGCCAACGGCGAGAu′CCGCAAG | | |
| | CGCCCCCu′GAu′CGAGACAAACGGCGAGACAGGCGAG | | |
| | Au′CGu′Gu′GGGACAAGGGCCGCGACu′u′CGCCACCGu′ | | |
| | GCGCAAGGu′GCu′GAGCAu′GCCCCAGGu′GAACAu′CGu | | |
| | ′GAAGAAGACCGAGGu′GCAGACCGGCGGCu′u′CAGCA | | |
| | AGGAGAGCAu′CCu′GCCCAAGCGCAACAGCGACAAGC | | |
| | u′GAu′CGCCCGCAAGAAGGACu′GGGACCCCAAGAAGu | | |
| | ′ACGGCGGCu′u′CGACAGCCCCACCGu′GGCCu′ACAGC | | |
| | Gu′GCu′GGu′GGu′GGCCAAGGu′GGAGAAGGGCAAGAG | | |
| | CAAGAAGCu′CAAGAGCGu′GAAGGAGCu′GCu′GGGCA | | |
| | u′CACCAu′CAu′GGAGCGCAGCAGCu′u′CGAGAAGAAC | | |
| | CCCAu′CGACu′u′CCu′GGAGGCCAAGGGCu′ACAAGGA | | |
| | GGu′GAAGAAGGACCu′GAu′CAu′CAAGCu′GCCCAAGu′ | | |
| | ACAGCCu′Gu′u′CGAGCu′GGAGAACGGCCGCAAGCGC | | |
| | Au′GCu′GGCCAGCGCCGGCGAGCu′GCAGAAGGGCAAC | | |
| | GAGCu′GGCCCu′GCCCAGCAAGu′ACGu′GAACu′u′CCu′ | | |
| | Gu′ACCu′GGCCAGCCACu′ACGAGAAGCu′GAAGGGCA | | |
| | GCCCCGAGGACAACGAGCAGAAGCAGCu′Gu′u′CGu′G | | |
| | GAGCAGCACAAGCACu′ACCu′GGACGAGAu′CAu′CGA | | |
| | GCAGAu′CAGCGAGu′u′CAGCAAGCGCGu′GAu′CCu′GG | | |
| | CCGACGCCAACCu′GGACAAGGu′GCu′GAGCGCCu′ACA | | |
| | ACAAGCACCGCGACAAGCCCAu′CCGCGAGCAGGCCG | | |
| | AGAACAu′CAu′CCACCu′Gu′u′CACCCu′GACCAACCu′G | | |
| | GGCGCCCCCGCCGCCu′u′CAAGu′ACu′u′CGACACCACC | | |
| | Au′CGACCGCAAGCGCu′ACACCAGCACCAAGGAGGu′G | | |
| | Cu′GGACGCCACCCu′GAu′CCACCAGAGCAu′CACCGGC | | |
| | Cu′Gu′ACGAGACACGCAu′CGACCu′GAGCCAGCu′GGG | | |
| | CGGCGACGAGGGCGCCGACAAGCGCACCGCCGACGG | | |
| | CAGCGAGu′u′CGAGAGCCCCAAGAAGAAGCGCAAGGu | | |
| | ′Gu′AAAGu′Gu′CCAGACCAu′u′Gu′Cu′u′CCAACCCCAG | | |
| | Cu′GGCCu′Cu′AGAACACCCACu′GGCCAGu′CCu′AGAG | | |
| | Cu′CCu′Gu′CCCu′ACCCACu′Cu′u′u′GCu′ACAAu′AAAu′G | | |
| | Cu′GAAu′GAAu′CCAu′Cu′AGA | | |
| | MSEVEFSHEYWMRHALTLAKRARDEREVPVGAVLVLN | Protein | 702 |
| | NRVIGEGWNRAIGLHDPTAHAEIMALRQGGLVMQNYRL | | |
| | IDATLYVTFEPCVMCAGAMIHSRIGRVVFGVRNAKTGA | | |
| | AGSLMDVLHHPGMNHR VEITEGILADECAALLCRFFRMP | | |
| | RRVFNAQKKAQSSTDSGGSSGGSSGSETPGTSESATPESS | | |
| | GGSSGGSDKKYSIGLAIGTNSVGWAVITDEYKVPSKKFK | | |
| | VLGNTDRHSIKKNLIGALLFDSGETAEATRLKRTARRRY | | |
| | TRRKNRICYLQEIFSNEMAKVDDSFFHRLEESFLVEEDKK | | |
| | HERHPIFGNIVDEVAYHEKYPTIYHLRKKLVDSTDKADL | | |
| | RLIYLALAHMIKFRGHFLIEGDLNPDNSDVDKLFIQLVQT | | |
| | YNQLFEENPINASGVDAKAILSARLSKSRRLENLIAQLPG | | |
| | EKKNGLFGNLIALSLGLTPNFKSNFDLAEDAKLQLSKDT | | |
| | YDDDLDNLLAQIGDQYADLFLAAKNLSDAILLSDILRVN | | |
| | TEITKAPLSASMIKRYDEHHQDLTLLKALVRQQLPEKYK | | |
| | EIFFDQSKNGYAGYIDGGASQEEFYKFIKPILEKMDGTEE | | |
| | LLVKLNREDLLRKQRTFDNGSIPHQIHLGELHAILRRQED | | |
| | FYPFLKDNREKIEKILTFRIPYYVGPLARGNSRFAWMTRK | | |
| | SEETITPWNFEEVVDKGASAQSFIERMTNFDKNLPNEKV | | |
| | LPKHSLLYEYFTVYNELTKVKYVTEGMRKPAFLSGEQK | | |
| | KAIVDLLFKTNRKVTVKQLKEDYFKKIECFDSVEISGVED | | |
| | RFNASLGTYHDLLKIIKDKDFLDNEENEDILEDIVLTLTLF | | |
| | EDREMIEERLKTYAHLFDDKVMKQLKRRRYTGWGRLSR | | |
| | KLINGIRDKQSGKTILDFLKSDGFANRNFMQLIHDDSLTF | | |
| | KEDIQKAQVSGQGDSLHEHIANLAGSPAIKKGILQTVKV | | |
| | VDELVKVMGRHKPENIVIEMARENQTTQKGQKNSRERM | | |
| | KRIEEGIKELGSQILKEHPVENTQLQNEKLYLYYLQNGR | | |
| | DMYVDQELDINRLSDYDVDHIVPQSFLKDDSIDNKVLTR | | |
| | SDKNRGKSDNVPSEEVVKKMKNYWRQLLNAKLITQRKF | | |
| | DNLTKAERGGLSELDKAGFIKRQLVETRQITKHVAQILD | | |
| | SRMNTKYDENDKLIREVKVITLKSKLVSDFRKDFQFYKV | | |
| | REINNYHHAHDAYLNAVVGTALIKKYPKLESEFVYGDY | | |
| | KVYDVRKMIAKSEQEIGKATAKYFFYSNIMNFFKTEITL | | |
| | ANGEIRKRPLIETNGETGEIVWDKGRDFATVRKVLSMPQ | | |
| | VNIVKKTEVQTGGFSKESILPKRNSDKLIARKKDWDPKK | | |
| | YGGFDSPTVAYSVLVVAKVEKGKSKKLKSVKELLGITIM | | |
| | ERSSFEKNPIDFLEAKGYKEVKKDLIIKLPKYSLFELENGR | | |
| | KRMLASAGELQKGNELALPSKYVNFLYLASHYEKLKGS | | |
| | PEDNEQKQLFVEQHKHYLDEIIEQISEFSKRVILADANLD | | |
| | KVLSAYNKHRDKPIREQAENIIHLFTLTNLGAPAAFKYFD | | |
| | TTIDRKRYTSTKEVLDATLIHQSITGLYETRIDLSQLGGDE | | |
| | GADKRTADGSEFESPKKKRKV | | |
| 5′ UTR | AGGACu′CCu′CCCCAu′CCu′Cu′CCCu′Cu′Gu′CCCu′Cu′G | mRNA | 703 |
| | u′CCCu′Cu′GACCCu′GCACu′Gu′CCCAGCACC | | |
| TadA | Au′GAGCGAGGu′GGAGu′u′CAGCCACGAGu′ACu′GGAu′ | mRNA | 704 |
| | GCGCCACGCCCu′GACCCu′GGCCAAGCGCGCCCGCGA | | |
| | CGAGCGCGAGGu′GCCCGu′GGGCGCCGu′GCu′GGu′GCu | | |
| | ′GAACAACCGCGu′GAu′CGGCGAGGGCu′GGAACCGCG | | |
| | CCAu′CGGCCu′GCACGACCCCACCGCCCACGCCGAGAu | | |
| | ′CAu′GGCCCu′GCGCCAGGGCGGCCu′GGu′GAu′GCAGA | | |
| | ACu′ACCGCCu′GAu′CGACGCCACCCu′Gu′ACGu′GACCu | | |
| | ′u′CGAGCCCu′GCGu′GAu′Gu′GCGCCGGCGCCAu′GAu′C | | |
| | CACAGCCGCAu′CGGCCGCGu′GGu′Gu′u′CGGCGu′GCG | | |
| | CAACGCCAAGACCGGCGCCGCCGGCAGCCu′GAu′GGA | | |
| | CGu′GCu′GCACCACCCCGGCAu′GAACCACCGCGu′GGA | | |
| | GAu′CACCGAGGGCAu′CCu′GGCCGACGAGu′GCGCCGC | | |
| | CCu′GCu′Gu′GCCGCu′u′Cu′u′CCGCAu′GCCCCGCCGCGu | | |
| | ′Gu′u′CAACGCCCAGAAGAAGGCCCAGAGCAGCACCG | | |
| | AC | | |
| | MSEVEFSHEYWMRHALTLAKRARDEREVPVGAVLVLN | Protein | 705 |
| | NRVIGEGWNRAIGLHDPTAHAEIMALRQGGLVMQNYRL | | |
| | IDATLYVTFEPCVMCAGAMIHSRIGRVVFGVRNAKTGA | | |
| | AGSLMDVLHHPGMNHRVEITEGILADECAALLCRFFRMP | | |
| | RRVFNAQKKAQSSTD | | |
| Linker | AGCGGCGGCAGCAGCGGCGGCAGCAGCGGCAGCGAG | mRNA | 692 |
| between | ACACCCGGCACCAGCGAGAGCGCCACCCCCGAGAGCA | | |
| TadA | GCGGCGGCAGCAGCGGCGGCAGC | | |
| and Cas9 | SGGSSGGSSGSETPGTSESATPESSGGSSGGS | Protein | 693 |
| nickase | | | |
| Cas9 | GACAAGAAGu′ACAGCAu′CGGCCu′GGCCAu′CGGCACC | mRNA | 706 |
| nickase | AACAGCGu′GGGCu′GGGCCGu′GAu′CACCGACGAGu′A | | |
| | CAAGGu′GCCCAGCAAGAAGu′u′CAAGGu′GCu′GGGCA | | |
| | ACACCGACCGCCACAGCAu′CAAGAAGAACCu′GAu′CG | | |
| | GCGCCCu′GCu′Gu′u′CGACAGCGGCGAGACAGCCGAGG | | |
| | CCACCCGCCu′GAAGCGCACCGCCCGCCGCCGCu′ACAC | | |
| | CCGCCGCAAGAACCGCAu′Cu′GCu′ACCu′GCAGGAGAu | | |
| | ′Cu′u′CAGCAACGAGAu′GGCCAAGGu′GGACGACAGCu′ | | |
| | u′Cu′u′CCACCGCCu′GGAGGAGAGCu′u′CCu′GGu′GGAG | | |
| | GAGGACAAGAAGCACGAGCGCCACCCCAu′Cu′u′CGGC | | |
| | AACAu′CGu′GGACGAGGu′GGCCu′ACCACGAGAAGu′A | | |
| | CCCCACCAu′Cu′ACCACCu′GCGCAAGAAGCu′GGu′GGA | | |
| | CAGCACCGACAAGGCCGACCu′GCGCCu′GAu′Cu′ACCu′ | | |
| | GGCCCu′GGCCCACAu′GAu′CAAGu′u′CCGCGGCCACu′u | | |
| | ′CCu′GAu′CGAGGGCGACCu′GAACCCCGACAACAGCG | | |
| | ACGu′GGACAAGCu′Gu′u′CAu′CCAGCu′GGu′GCAGACC | | |
| | u′ACAACCAGCu′Gu′u′CGAGGAGAACCCCAu′CAACGC | | |
| | CAGCGGCGu′GGACGCCAAGGCCAu′CCu′GAGCGCCCG | | |
| | CCu′GAGCAAGAGCCGCCGCCu′GGAGAACCu′GAu′CGC | | |
| | CCAGCu′GCCCGGCGAGAAGAAGAACGGCCu′Gu′u′CG | | |
| | GCAACCu′GAu′CGCCCu′GAGCCu′GGGCCu′GACCCCCA | | |
| | ACu′u′CAAGAGCAACu′u′CGACCu′GGCCGAGGACGCC | | |
| | AAGCu′GCAGCu′GAGCAAGGACACCu′ACGACGACGAC | | |
| | Cu′GGACAACCu′GCu′GGCCCAGAu′CGGCGACCAGu′A | | |
| | CGCCGACCu′Gu′u′CCu′GGCCGCCAAGAACCu′GAGCGA | | |
| | CGCCAu′CCu′GCu′GAGCGACAu′CCu′GCGCGu′GAACA | | |
| | CCGAGAu′CACCAAGGCCCCCCu′GAGCGCCAGCAu′GA | | |
| | u′CAAGCGCu′ACGACGAGCACCACCAGGACCU′GACCC | | |
| | u′GCu′GAAGGCCCu′GGu′GCGCCAGCAGCu′GCCCGAG | | |
| | AAGu′ACAAGGAGAu′Cu′u′Cu′u′CGACCAGAGCAAGAA | | |
| | CGGCu′ACGCCGGCu′ACAu′CGACGGCGGCGCCAGCCA | | |
| | GGAGGAGu′u′Cu′ACAAGu′u′CAu′CAAGCCCAu′CCu′GG | | |
| | AGAAGAu′GGACGGCACCGAGGAGCu′GCu′GGu′GAAG | | |
| | Cu′GAACCGCGAGGACCu′GCu′GCGCAAGCAGCGCACC | | |
| | u′u′CGACAACGGCAGCAu′CCCCCACCAGAu′CCACCu′G | | |
| | GGCGAGCu′GCACGCCAu′CCu′GCGCCGCCAGGAGGAC | | |
| | u′u′Cu′ACCCCu′u′CCu′GAAGGACAACCGCGAGAAGAu′ | | |
| | CGAGAAGAu′CCu′GACCu′u′CCGCAu′CCCCu′ACu′ACGu | | |
| | ′GGGCCCCCu′GGCCCGCGGCAACAGCCGCu′u′CGCCu′ | | |
| | GGAu′GACCCGCAAGAGCGAGGAGACAAu′CACCCCCu′ | | |
| | GGAACu′u′CGAGGAGGu′GGu′GGACAAGGGCGCCAGC | | |
| | GCCCAGAGCu′u′CAu′CGAGCGCAu′GACCAACu′u′CGA | | |
| | CAAGAACCu′GCCCAACGAGAAGGu′GCu′GCCCAAGCA | | |
| | CAGCCu′GCu′Gu′ACGAGu′ACu′u′CACCGu′Gu′ACAACG | | |
| | AGCu′GACCAAGGu′GAAGu′ACGu′GACCGAGGGCAu′G | | |
| | CGCAAGCCCGCCu′u′CCu′GAGCGGCGAGCAGAAGAAG | | |
| | GCCAu′CGu′GGACCu′GCu′Gu′u′CAAGACCAACCGCAA | | |
| | GGu′GACCGu′GAAGCAGCu′GAAGGAGGACu′ACu′u′CA | | |
| | AGAAGAu′CGAGu′GCu′u′CGACAGCGu′GGAGAu′CAGC | | |
| | GGCGu′GGAGGACCGCu′u′CAACGCCAGCCu′GGGCACC | | |
| | u′ACCACGACCu′GCu′GAAGAu′CAu′CAAGGACAAGGA | | |
| | Cu′u′CCu′GGACAACGAGGAGAACGAGGACAu′CCu′GG | | |
| | AGGACAu′CGu′GCu′GACCCu′GACCCu′Gu′u′CGAGGAC | | |
| | CGCGAGAu′GAu′CGAGGAGCGCCu′GAAGACCu′ACGC | | |
| | CCACCu′Gu′u′CGACGACAAGGu′GAu′GAAGCAGCu′GA | | |
| | AGCGCCGCCGCu′ACACCGGCu′GGGGCCGCCu′GAGCC | | |
| | GCAAGCu′GAu′CAACGGCAu′CCGCGACAAGCAGAGCG | | |
| | GCAAGACCAu′CCu′GGACu′u′CCu′CAAGAGCGACGGCu | | |
| | ′u′CGCCAACCGCAACu′u′CAu′GCAGCu′GAu′CCACGAC | | |
| | GACAGCCu′GACCu′u′CAAGGAGGACAu′CCAGAAGGC | | |
| | CCAGGu′GAGCGGCCAGGGCGACAGCCu′GCACGAGCA | | |
| | CAu′CGCCAACCu′GGCCGGCAGCCCCGCCAu′CAAGAA | | |
| | GGGCAu′CCu′GCAGACCGu′GAAGGu′GGu′GGACGAGC | | |
| | u′GGu′GAAGGu′GAu′GGGCCGCCACAAGCCCGAGAAC | | |
| | Au′CGu′GAu′CGAGAu′GGCCCGCGAGAACCAGACCACC | | |
| | CAGAAGGGCCAGAAGAACAGCCGCGAGCGCAu′GAAG | | |
| | CGCAu′CGAGGAGGGCAu′CAAGGAGCu′GGGCAGCCA | | |
| | GAu′CCu′GAAGGAGCACCCCGu′GGAGAACACCCAGCu′ | | |
| | GCAGAACGAGAAGCu′Gu′ACCu′Gu′ACu′ACCu′GCAGA | | |
| | ACGGCCGCGACAu′Gu′ACGu′GGACCAGGAGCu′GGAC | | |
| | Au′CAACCGCCu′GAGCGACu′ACGACGu′GGACCACAu′ | | |
| | CGu′GCCCCAGAGCu′u′CCu′GAAGGACGACAGCAu′CG | | |
| | ACAACAAGGu′GCu′GACCCGCAGCGACAAGAACCGCG | | |
| | GCAAGAGCGACAACGu′GCCCAGCGAGGAGGu′GGu′G | | |
| | AAGAAGAu′GAAGAACu′ACu′GGCGCCAGCu′GCu′GAA | | |
| | CGCCAAGCu′GAu′CACCCAGCGCAAGu′u′CGACAACCu′ | | |
| | GACCAAGGCCGAGCGCGGCGGCCu′GAGCGAGCu′GGA | | |
| | CAAGGCCGGCu′u′CAu′CAAGCGCCAGCu′GGu′GGAGA | | |
| | CACGCCAGAu′CACCAAGCACGu′GGCCCAGAu′CCu′GG | | |
| | ACAGCCGCAu′GAACACCAAGu′ACGACGAGAACGACA | | |
| | AGCu′GAu′CCGCGAGGu′GAAGGu′GAu′CACCCu′CAAG | | |
| | AGCAAGCu′GGu′GAGCGACu′u′CCGCAAGGACu′u′CCA | | |
| | Gu′u′Cu′ACAAGGu′GCGCGAGAu′CAACAACu′ACCACC | | |
| | ACGCCCACGACGCCu′ACCu′GAACGCCGu′GGu′GGGCA | | |
| | CCGCCCu′GAu′CAAGAAGu′ACCCCAAGCu′GGAGAGCG | | |
| | AGu′u′CGu′Gu′ACGGCGACu′ACAAGGu′Gu′ACGACGu′G | | |
| | CGCAAGAu′GAu′CGCCAAGAGCGAGCAGGAGAu′CGG | | |
| | CAAGGCCACCGCCAAGu′ACu′u′Cu′u′Cu′ACAGCAACAu | | |
| | ′CAu′GAACu′u′Cu′u′CAAGACCGAGAu′CACCCu′GGCCA | | |
| | ACGGCGAGAu′CCGCAAGCGCCCCCu′GAu′CGAGACAA | | |
| | ACGGCGAGACAGGCGAGAu′CGu′Gu′GGGACAAGGGC | | |
| | CGCGACu′u′CGCCACCGu′GCGCAAGGu′GCu′GAGCAu′ | | |
| | GCCCCAGGu′GAACAu′CGu′GAAGAAGACCGAGGu′GC | | |
| | AGACCGGCGGCu′u′CAGCAAGGAGAGCAu′CCu′GCCCA | | |
| | AGCGCAACAGCGACAAGCu′GAu′CGCCCGCAAGAAGG | | |
| | ACu′GGGACCCCAAGAAGu′ACGGCGGCu′u′CGACAGCC | | |
| | CCACCGu′GGCCu′ACAGCGu′GCu′GGu′GGu′GGCCAAG | | |
| | Gu′GGAGAAGGGCAAGAGCAAGAAGCu′CAAGAGCGu′ | | |
| | GAAGGAGCu′GCu′GGGCAu′CACCAu′CAu′GGAGCGCA | | |
| | GCAGCu′u′CGAGAAGAACCCCAu′CGACu′u′CCu′GGAG | | |
| | GCCAAGGGCu′ACAAGGAGGu′GAAGAAGGACCu′GAu′ | | |
| | CAu′CAAGCu′GCCCAAGu′ACAGCCu′Gu′u′CGAGCu′GG | | |
| | AGAACGGCCGCAAGCGCAu′GCu′GGCCAGCGCCGGCG | | |
| | AGCu′GCAGAAGGGCAACGAGCu′GGCCCu′GCCCAGCA | | |
| | AGu′ACGu′GAACu′u′CCu′Gu′ACCu′GGCCAGCCACu′AC | | |
| | GAGAAGCu′GAAGGGCAGCCCCGAGGACAACGAGCAG | | |
| | AAGCAGCu′Gu′u′CGu′GGAGCAGCACAAGCACu′ACCu′ | | |
| | GGACGAGAu′CAu′CGAGCAGAu′CAGCGAGu′u′CAGCA | | |
| | AGCGCGu′GAu′CCu′GGCCGACGCCAACCu′GGACAAGG | | |
| | u′GCu′GAGCGCCu′ACAACAAGCACCGCGACAAGCCCA | | |
| | u′CCGCGAGCAGGCCGAGAACAu′CAu′CCACCu′Gu′u′C | | |
| | ACCCu′GACCAACCu′GGGCGCCCCCGCCGCCu′u′CAAG | | |
| | u′ACu′u′CGACACCACCAu′CGACCGCAAGCGCu′ACACC | | |
| | AGCACCAAGGAGGu′GCu′GGACGCCACCCu′GAu′CCAC | | |
| | CAGAGCAu′CACCGGCCu′Gu′ACGAGACACGCAu′CGAC | | |
| | Cu′GAGCCAGCu′GGGCGGCGAC | | |
| | DKKYSIGLAIGTNSVGWAVITDEYKVPSKKFKVLGNTDR | Protein | 707 |
| | HSIKKNLIGALLFDSGETAEATRLKRTARRRYTRRKNRIC | | |
| | YLQEIFSNEMAKVDDSFFHRLEESFLVEEDKKHERHPIFG | | |
| | NIVDEVAYHEKYPTIYHLRKKLVDSTDKADLRLIYLALA | | |
| | HMIKFRGHFLIEGDLNPDNSDVDKLFIQLVQTYNQLFEE | | |
| | NPINASGVDAKAILSARLSKSRRLENLIAQLPGEKKNGLF | | |
| | GNLIALSLGLTPNFKSNFDLAEDAKLQLSKDTYDDDLDN | | |
| | LLAQIGDQYADLFLAAKNLSDAILLSDILRVNTEITKAPL | | |
| | SASMIKRYDEHHQDLTLLKALVRQQLPEKYKEIFFDQSK | | |
| | NGYAGYIDGGASQEEFYKFIKPILEKMDGTEELLVKLNR | | |
| | EDLLRKQRTFDNGSIPHQIHLGELHAILRRQEDFYPFLKD | | |
| | NREKIEKILTFRIPYYVGPLARGNSRFAWMTRKSEETITP | | |
| | WNFEEVVDKGASAQSFIERMTNFDKNLPNEKVLPKHSLL | | |
| | YEYFTVYNELTKVKYVTEGMRKPAFLSGEQKKAIVDLL | | |
| | FKTNRKVTVKQLKEDYFKKIECFDSVEISGVEDRFNASL | | |
| | GTYHDLLKIIKDKDFLDNEENEDILEDIVLTLTLFEDREMI | | |
| | EERLKTYAHLFDDKVMKQLKRRRYTGWGRLSRKLINGI | | |
| | RDKQSGKTILDFLKSDGFANRNFMQLIHDDSLTFKEDIQ | | |
| | KAQVSGQGDSLHEHIANLAGSPAIKKGILQTVKVVDELV | | |
| | KVMGRHKPENIVIEMARENQTTQKGQKNSRERMKRIEE | | |
| | GIKELGSQILKEHPVENTQLQNEKLYLYYLQNGRDMYV | | |
| | DQELDINRLSDYDVDHIVPQSFLKDDSIDNKVLTRSDKN | | |
| | RGKSDNVPSEEVVKKMKNYWRQLLNAKLITQRKFDNLT | | |
| | KAERGGLSELDKAGFIKRQLVETRQITKHVAQILDSRMN | | |
| | TKYDENDKLIREVKVITLKSKLVSDFRKDFQFYKVREIN | | |
| | NYHHAHDAYLNAVVGTALIKKYPKLESEFVYGDYKVY | | |
| | DVRKMIAKSEQEIGKATAKYFFYSNIMNFFKTEITLANGE | | |
| | IRKRPLIETNGETGEIVWDKGRDFATVRKVLSMPQVNIV | | |
| | KKTEVQTGGFSKESILPKRNSDKLIARKKDWDPKKYGGF | | |
| | DSPTVAYSVLVVAKVEKGKSKKLKSVKELLGITIMERSS | | |
| | FEKNPIDFLEAKGYKEVKKDLIIKLPKYSLFELENGRKRM | | |
| | LASAGELQKGNELALPSKYVNFLYLASHYEKLKGSPEDN | | |
| | EQKQLFVEQHKHYLDEIIEQISEFSKRVILADANLDKVLS | | |
| | AYNKHRDKPIREQAENIIHLFTLTNLGAPAAFKYFDTTID | | |
| | RKRYTSTKEVLDATLIHQSITGLYETRIDLSQLGGD | | |
| Linker | GAGGGCGCCGAC | mRNA | 696 |
| between | EGAD | Protein | 697 |
| Cas9 | | | |
| nickase | | | |
| and NLS | | | |
| Nuclear | AAGCGCACCGCCGACGGCAGCGAGu′u′CGAGAGCCCC | mRNA | 708 |
| local- | AAGAAGAAGCGCAAGGu′G | | |
| ization | KRTADGSEFESPKKKRKV | Protein | 709 |
| sequence | | | |
| (NLS) | | | |
| 3′ UTR | AGu′Gu′CCAGACCAu′u′Gu′Cu′u′CCAACCCCAGCu′GGC | mRNA | 710 |
| | Cu′Cu′AGAACACCCACu′GGCCAGu′CCu′AGAGCu′CCu′ | | |
| | Gu′CCCu′ACCCACu′Cu′u′u′GCu′ACAAu′AAAu′GCu′GAA | | |
| | u′GAAu′CCAu′Cu′AGA | | |

The mutations at amino acid positions 691 and 1135 of the nCas9 component and their corresponding nucleotide sequences are indicated as bold and underlined.
u′: N¹-methylpseudouridine
The first nucleotide in the 5′ UTR has a 2′-O-methyl modification.

### Example 14. Exemplary LNP Formulations

LNP formulations are shown in Table 11. GalNAc-LNP formulation parameters and characteristics using 1004 as GalNAc-lipid, except 7-95 through 7-98, and 7-109 through 7-111. 7-95 was 1044; 7-96 and 7-109 were 1002; 7-97 and 7-110 were 1014; and 7-98 and 7-111 were 1078. 1004 or another indicated GalNAc-lipid was added to the formulation process in the mixture of lipids (e.g. ionizable lipid, stealth lipid and helper lipid) in ethanol, except where indicated post addition was used. The amino lipid 502 was used for all LNP IDs except 7-78, and 7-80 to 7-86, which used amino lipid VL422. The LNP composition varies in all examples. Different formulation buffers of choice at different pH were used for the LNP preparation whereas final formulation buffer pH was maintained in 6-8 range. All the GalNAc-LNPs were carrying mRNA MA004 and gRNA GA256 at 1:1 weight ratio, except 7-64, 7-65, 7-87 through 7-98, and 7-105 through 7-111 where the gRNA used was gRNA GA260, and 7-78 to 7-86 which used gRNA GA257. For the preparation of LNP 7-77, cross mixing and TFF were used.

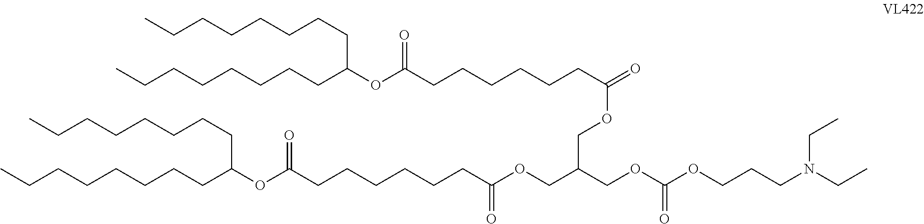 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

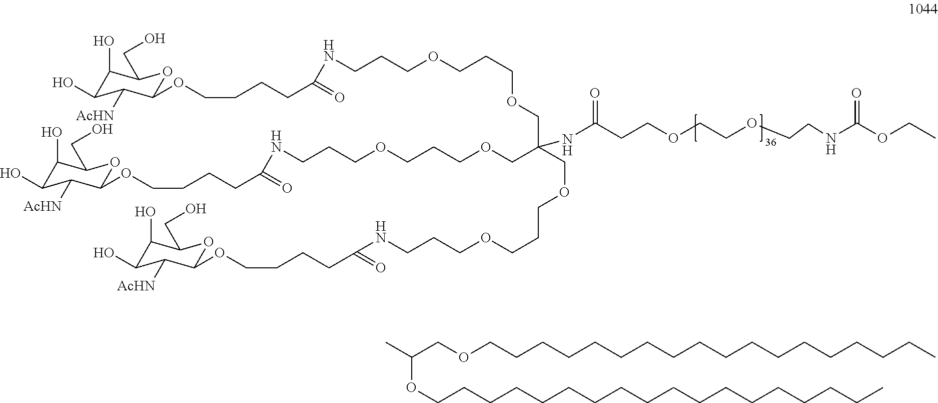

It is to be understood that lipid mix addition method refers to a method, wherein one channel/line was used for delivering the lipid mixture (including but not limited to: ionizable lipid, stealth lipid, and helper lipid) to a mixing chamber. The GalNAc-Lipid 1004 was included in the lipid excipients channel such that the final mol % of the GalNAc-Lipid in the LNPs was in the range of 0.01-10%. The second channel/line was used to deliver the cargo dissolved in an aqueous solution (including but not limited to: guide RNA and mRNA). The lipid and RNA channels mixed in a mixing device of desired geometry (e.g. T mixer, Cross Mixer, among others). The dilution buffer was then introduced through a downstream second mixer (T mixer, cross mixer, among others) to the resulting ethanol/aqueous mix to produce the desired LNPs. The LNPs thus produced were allowed to stand for 1 min to 120 min before being buffer exchanged into the final formulation buffer and stored. The buffer exchange occurred via PD-10 column, dialysis, or TFF.

It is to be understood that Inline Dilution method refers to a method, wherein one channel delivered the lipid mixture (including but not limited to: ionizable lipid, stealth lipid, helper lipid, etc.). The second channel/line was used to deliver the cargo dissolved in an aqueous solution (including but not limited to: guide RNA and mRNA). The lipid and RNA channels mix in a mixing device of desired geometry (e.g. T mixer, Cross Mixer, among others). The GalNAc-Lipid 1004 was included in the dilution buffer such that the final mol % of the GalNAc-Lipid in the LNPs is in the range of 0.01-10%. The dilution buffer containing the desired amount of the GalNAc-Lipid was then immediately introduced to the resulting ethanol/aqueous mix through a third channel to produce the desired LNPs. The LNPs were allowed to stand for 1 min to 120 min before being buffer exchanged into the final formulation buffer and was stored. The buffer exchange occurred via PD-10 column, dialysis, or TFF.

TABLE 11

| | Excipient mol % | Drug | | Z | | | 1004 |
|---|---|---|---|---|---|---|---|
| LNP | 502/Cholesterol/ | Product | | average | | Encapsulation | Addition |
| ID | DSPC/507/1004 | Buffer | Storage | (nm) | PDI | (%) | Method |
| 7-42 | 55/38.4/4.7/1.6/0.3 | Tris | 2-4C | 105.405 | 0.082 | 95.57 | Inline Dilution |
| 7-43 | 55/37.7/4.7/2.1/0.5 | Tris | 2-4C | 89.59 | 0.048 | 95.16 | Inline Dilution |
| 7-44 | 55/38.6/4.7/1.6/0.1 | PBS | 2-4C | 96.47 | 0.021 | 94.48 | Lipid Mix |
| 7-45 | 55/38.5/4.7/1.6/0.2 | PBS | 2-4C | 90.54 | 0.094 | 94.76 | Lipid Mix |
| 7-46 | 55/38.4/4.7/1.6/0.3 | PBS | 2-4C | 92.28 | 0.077 | 95.35 | Lipid Mix |
| 7-47 | 55/38.3/4.7/1.6/0.4 | PBS | 2-4C | 92.03 | 0.01 | 95.6 | Lipid Mix |
| 7-48 | 55/38.2/4.7/1.6/0.5 | PBS | 2-4C | 83.4 | 0.065 | 94.99 | Lipid Mix |
| 7-55 | 55/38.2/4.7/2.1/0.5 | PBS | 2-4C | 88.37 | 0.051 | 95.1 | Lipid Mix |
| 7-49 | 55/38.6/4.7/1.6/0.1 | PBS | 2-4C | 124.1 | 0.0918 | 93.6 | Post Addition |
| 7-50 | 55/38.65/4.7/1.6/0.50 | PBS | 2-4C | 138 | 0.1133 | 93.4 | Lipid Mix |
| 7-51 | 55/38.95/4.7/1.3/0.05 | PBS | 2-4C | 117.7 | 0.01225 | 92.8 | Lipid Mix |
| 7-52 | 55/38.9/4.7/1.3/0.1 | PBS | 2-4C | 133.2 | 0.04513 | 91.4 | Lipid Mix |
| 7-53 | 55/38.8/4.7/1.3/0.2 | PBS | 2-4C | 117.2 | 0.02134 | 93.5 | Lipid Mix |
| 7-54 | 55/38.7/4.7/1.3/0.3 | PBS | 2-4C | 107 | 0.0641 | 93.4 | Lipid Mix |
| 7-56 | 55/38.15/4.7/2.1/0.05 | PBS | 2-4C | 73.8 | 0.01 | 95.14 | Lipid Mix |
| 7-57 | 55/38.675/4.7/1.6/0.025 | PBS | 2-4C | 86.04 | 0.022 | 94.18 | Lipid Mix |
| 7-58 | 55/38.69/4.7/1.6/0.01 | PBS | 2-4C | 87.2 | 0.036 | 93.9 | Lipid Mix |
| 7-59 | 55/38.975/4.7/1.3/0.025 | PBS | 2-4C | 93.24 | 0.06 | 93.91 | Lipid Mix |
| 7-64 | 55/38.65/4.7/1.6/0.05 | PBS | 2-4C | 103.4 | 0.01883 | 95.97 | Lipid Mix |
| 7-65 | 55/38.15/4.7/2.1/0.05 | Tris | 2-4C | 102.2 | 0.01135 | 92.55 | Lipid Mix |
| 7-66 | 55/38.15/4.7/2.1/0.05 | Tris | 2-4C | 101 | 0.05521 | 95.51 | Lipid Mix |
| 7-67 | 55/37.25/4.7/3/0.05 | Tris | 2-4C | 91.23 | 0.03484 | 90.36 | Lipid Mix |
| 7-68 | 52.4/34.55/10/3/0.05 | Tris | 2-4C | 86.79 | 0.04927 | 91.13 | Lipid Mix |
| 7-69 | 55/38.15/4.7/3/0.05 | Tris | 2-4C | 103.5 | 0.007838 | 89.49 | Lipid Mix |
| 7-70 | 52.4/34.55/10/3/0.05 | Tris | 2-4C | 70.94 | 0.07394 | 93.09 | Lipid Mix |
| 7-71 | 50/37.95/9/3/0.05 | Tris | 2-4C | 72.3 | 0.04853 | 92.23 | Lipid Mix |
| 7-72 | 47/40.95/9/3/0.05 | Tris | 2-4C | 66.33 | 0.05201 | 92.98 | Lipid Mix |
| 7-74 | 50/37.95/9/3/0.05 | Tris | 2-4C | 67.91 | 0.08725 | 91.48 | Lipid Mix |
| 7-75 | 50/37.95/9/3/0.05 | Tris | 2-4C | 82.7 | 0.2582 | 91.36 | Lipid Mix |
| 7-77 | 50/37.95/9/3/0.05 | Tris | 2-4C | 70.22 | 0.06838 | 91.57 | Lipid Mix |
| 7-78 | 50/38/9/3/0 | Tris | 2-4C | 66.85 | 0.06473 | 93.53 | Control |
| 7-79 | 50/37.95/9/3/0.05 | Tris | 2-4C | 70.9 | 0.1017 | 95.23 | Lipid Mix |
| 7-80 | 50/37.99/9/3/0.01 | Tris | 2-4C | 66.91 | 0.05535 | 93.6 | Lipid Mix |
| 7-81 | 50/37.95/9/3/0.05 | Tris | 2-4C | 66.96 | 0.05259 | 93.89 | Lipid Mix |
| 7-82 | 50/37.9/9/3/0.1 | Tris | 2-4C | 65.32 | 0.05412 | 93.28 | Lipid Mix |
| 7-83 | 50/37.7/9/3/0.3 | Tris | 2-4C | 64.83 | 0.07319 | 93.33 | Lipid Mix |
| 7-84 | 50/37.5/9/3/0.5 | Tris | 2-4C | 63 | 0.09219 | 93.21 | Lipid Mix |
| 7-85 | 50/37/9/3/1 | Tris | 2-4C | 62.7 | 0.1178 | 92.19 | Lipid Mix |
| 7-86 | 50/36/9/3/2 | Tris | 2-4C | 60.86 | 0.06607 | 91.41 | Lipid Mix |
| 7-87 | 55/38.2/4.7/2.1/0 | Tris | 2-4C | 106.6 | 0.008889 | 94.85 | Control |
| 7-88 | 55/38.15/4.7/2.1/0.05 | Tris | 2-4C | 120.6 | 0.01052 | 94.27 | Post Addition |
| 7-89 | 55/37.7/4.7/2.1/0.5 | Tris | 2-4C | 92.61 | 0.01561 | 95.7 | Post Addition |
| 7-90 | 55/38.19/4.7/2.1/0.01 | Tris | 2-4C | 98.35 | 0.01628 | 95.01 | Lipid Mix |
| 7-91 | 55/38.15/4.7/2.1/0.05 | Tris | 2-4C | 98.61 | 0.01343 | 94.91 | Lipid Mix |
| 7-92 | 55/37.95/4.7/2.1/0.25 | Tris | 2-4C | 95.98 | 0.01044 | 95.84 | Lipid Mix |
| 7-93 | 55/37.7/4.7/2.1/0.5 | Tris | 2-4C | 89.63 | 0.02471 | 96.15 | Lipid Mix |
| 7-94 | 55/37.2/4.7/2.1/1 | Tris | 2-4C | 84.69 | 0.02132 | 95.28 | Lipid Mix |
| 7-95 | 55/38.15/4.7/2.1/0.05 | Tris | 2-4C | 100.4 | 0.0228 | 94.27 | Lipid Mix |
| 7-96 | 55/38.15/4.7/2.1/0.05 | Tris | 2-4C | 100.6 | 0.007328 | 95.09 | Lipid Mix |
| 7-97 | 55/38.15/4.7/2.1/0.05 | Tris | 2-4C | 94.89 | 0.02021 | 94.41 | Lipid Mix |
| 7-98 | 55/38.15/4.7/2.1/0.05 | Tris | 2-4C | 94.82 | 0.01814 | 94.86 | Lipid Mix |
| 7-105 | 55/38.2/4.7/2.1/0 | Tris | 2-4C | 99.27 | 0.02149 | 91.7 | Control |
| 7-106 | 55/38.2/4.7/2.1/0 | Tris | 2-4C | 107.6 | 0.003511 | 93.20 | Control |
| 7-107 | 55/38.15/4.7/2.1/0.05 | Tris | 2-4C | 100.4 | 0.0343 | 93.59 | Lipid Mix |
| 7-108 | 55/37.7/4.7/2.1/0.5 | Tris | 2-4C | 93.59 | 0.04789 | 92.33 | Lipid Mix |
| 7-109 | 55/38.15/4.7/2.1/0.05 | Tris | 2-4C | 100.6 | 0.0288 | 92.48 | Lipid Mix |
| 7-110 | 55/38.15/4.7/2.1/0.05 | Tris | 2-4C | 98.78 | 0.03208 | 92.86 | Lipid Mix |
| 7-111 | 55/38.15/4.7/2.1/0.05 | Tris | 2-4C | 100.9 | 0.002 | 92.65 | Lipid Mix |
| 7-112 | 47.1/46.1/4.7/2.1/0 | PBS | 2-4C | 85.5 | 0.0313 | 96.3 | Control |
| 7-113 | 55/38.2/4.7/2.1/0 | PBS | 2-4C | 84.8 | 0.0263 | 96.7 | Control |
| 7-114 | 55/38.2/4.7/2.1/0 | PBS | 2-4C | 90.35 | 0.01 | 96.4 | Control |
| 7-115 | 55/38.2/4.7/2.1/0 | PBS | 2-4C | 87.21 | 0.0077 | 94.10 | Control |
| 7-116 | 55/38.6/4.7/1.6/0.1 | PBS | 2-4C | 107.5 | 0.297 | 94.00 | Lipid Mix |
| 7-117 | 55/38.2/4.7/2.1/0 | PBS | 2-4C | 89 | 0.01 | 93.74 | Control |
| 7-118 | 55/38.65/4.7/1.6/0.05 | PBS | 2-4C | 83.2 | 0.024 | 94.26 | Lipid Mix |
| 7-119 | 55/38.95/4.7/1.3/0.05 | PBS | 2-4C | 94.8 | 0.06 | 93.33 | Lipid Mix |
| 7-120 | 55/38.2/4.7/2.1/0 | PBS | 2-4C | 89.77 | 0.03029 | 93.32 | Control |
| 7-121 | 55/38.65/4.7/1.6/0.05 | PBS | 2-4C | 101.1 | 0.03086 | 92.21 | Lipid Mix |
| 7-122 | 55/38.2/4.7/2.1/0 | Tris | 2-4C | 104.7 | 0.01344 | 90.52 | Control |
| 7-123 | 55/37.3/4.7/3/0 | Tris | 2-4C | 90.9 | 0.03435 | 90.91 | Control |
| 7-124 | 52.4/34.6/10/3/0 | Tris | 2-4C | 87.66 | 0.01843 | 91.54 | Control |
| 7-125 | 55/38.15/4.7/2.1/0.05 | PBS | 2-4C | 82.07 | 0.02038 | 96.00 | Lipid Mix |
| 7-126 | 55/38.2/4.7/2.1/0 | Tris | 2-4C | 103.7 | 0.01282 | 90.98 | Control |
| 7-127 | 52.4/34.6/10/3/0 | Tris | 2-4C | 71.56 | 0.03463 | 93.43 | Control |
| 7-128 | 50/38/9/3/0 | Tris | 2-4C | 72.09 | 0.03283 | 92.57 | Control |
| 7-129 | 47/41/9/3/0 | Tris | 2-4C | 64.48 | 0.07433 | 93.88 | Control |
| 7-130 | 50/38/9/3/0 | Tris | 2-4C | 67.24 | 0.07985 | 90.26 | Control |

TABLE 13
Lipids and PEG-Lipids excipients used for LNP preparations

| Compound | |
|---|---|
| number | Structure |
| 502 | 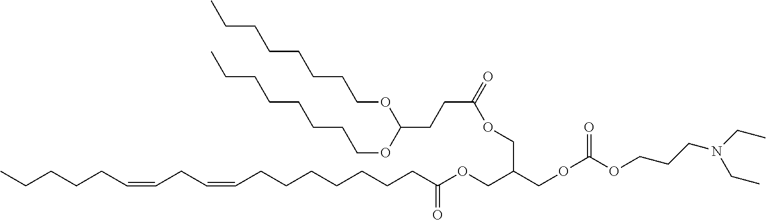 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 507 | 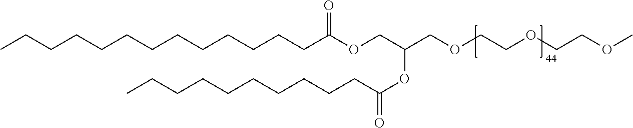 |

### Methods

### Plating, Culturing, and Transfection of Primary Hepatocytes.

Primary human liver hepatocytes (PHH) and primary cynomolgus liver hepatocytes (PCH) from BioIVT were cultured per the manufacturer's protocol. Briefly, primary human hepatocytes and primary cyno hepatocytes were obtained as frozen aliquots from BioIVT. Two lots of primary human hepatocytes, each derived from a de-identified individual donor, were used for the experiments: STL was used for initial screening experiments; IRZ was used for potency dose-response and off-target experiments. For experiments with primary cyno hepatocytes, the UMP lot was used. Following the manufacturer's instructions, cells were thawed and rinsed with pre-warmed INVITROGRO HT medium prior to centrifugation at 4° C. for either 50 g for 5 min (for PCH) or at 100 g for 10 min (for PCH). The supernatant was discarded, the pelleted cells resuspended in plating medium, INVITROGRO CP, and plated onto either 24- or 48-well plates, which were either pre-coated or had been coated with bovine collagen overnight. For each 24-well plate, 1 vial of cells was used to achieve a density of approximately 350,000 cells/well; for each 48-well plate, 2 vials of cells were used to achieve a density of approximately 200,000 cells/well. Plated cells were allowed to settle and adhere for at least 4 h, but at times up to 24 hours, in a tissue culture incubator at 37° C. under 5% CO₂ atmosphere. After incubation, cells were checked for monolayer formation. The incubating media were then replaced with fresh hepatocyte maintenance media (complete INVITROGRO medium). The cells thus became ready for transfection. Cells were transfected either using a lipofectamine transfection agent or with LNP.

When using the lipofectamine reagent MessengerMax (ThermoFisher) for transfection of primary hepatocytes, gRNAs or gRDNAs were co-transfected with an equivalent amount of in vitro transcribed ABE8.8 mRNA (1:1 ratio by molecular weight) via, at doses ranging from 1,250-5,000 ng/mL of total RNA. Solution A: desired amount of guide RNA or guide RDNA was mixed with 1:1 wt ratio of mRNA in OptiMEM. Solution B: MessengerMAX in OptiMEM, incubated for 10 min at room temperature. After mixing solutions A and B, the mixture was incubated at room temperature for 5 min. In the initial screening experiments, this mix was then serially diluted 1:2 into OptiMEM for a dose titration. Regardless, 60 μL of the respective solution was added dropwise to each well of cells. The cells were then allowed to remain at 37° C. for 3 days. Cells were harvested and prepared for genomic DNA extraction using either a Thermo Kingfisher (for 48-well plate experiments), or Qiagen DNEasy blood and Tissue Kit (for 24-well plate experiments), per manufacturer's instructions, and quantified using a Qubit 3.0 Fluorometer.

Transfections were also performed with LNPs. The desired LNPs were mixed with plating media, containing 10% cynomolgous monkey serum prior to plating, at concentrations dependent on the desired top dose of test article, which was between 10,000 and 45,000 ng/TA/mL. When necessary, this mix was serially diluted 1:1.5 or 1:2 into media. LNP dilution mixes were incubated at 37° C. for 15 min and then added to plated cells, at doses ranging from 45,000 to 69 ng/TA/mL, after the previous media had been aspirated. The cells were then allowed to remain at 37° C. for 3 days. Cells were harvested and prepared for genomic DNA extraction using either a Thermo Kingfisher or Promega Maxwell RSC 48 (for 48-well plate experiments), or Qiagen DNEasy blood and Tissue Kit (for 6-well and 24-well plate experiments), per manufacturer's instructions, and quantified using a Qubit 3.0 Fluorometer. For OGM, genomic DNA was extracted following Bionano Genomic's protocol for high molecular weight (HMW) gDNA extraction. Pellets of 1.5×106 cells were resuspended in 40 μL DNA Stabilizing Buffer followed by Proteinase K digestion, RNaseA treatment and lysis in 220 μL Lysis and Binding Buffer (LBB). Genomic DNA was isolated and bound to Nanobind Disks, washed, and eluted by 65 μL Elution Buffer. Genomic DNA was then homogenized overnight and quantified using the Qubit Broad Range dsDNA Assay Kit.

### Targeted Amplicon Sequencing.

To assess for on-target and off-target editing, 10 ng of genomic DNA input was used for PCR amplification with Q5 HotStart polymerase (NEB) and primers specific to the target genomic sites-designed with Primer3 v.4.1.0 (https://primer3.ut.ee/)—with 5′ Nextera adaptor overhang sequences. The primer sequences were as follows:

TABLE 12
Primer Sequences

| | | Forward | Forward Primer | SEQ | Reverse | Reverse Primer | SEQ |
|---|---|---|---|---|---|---|---|
| | | Primer | Sequence | ID | Primer | Sequence | ID |
| Species | Target | Name | (5′ to 3′) | NO. | Name | (5′ to 3′) | NO. |
| Human | On- | GA0100_ | TCGTCGGCAGCGT | 711 | GA0100_ | GTCTCGTGGG | 714 |
| | target | OT_B1+ | CAGATGTGTATA | | OT_B1− | CTCGGAGATG | |
| | | | AGAGACAGAATG | | | TGTATAAGAG | |
| | | | TGGGAAGATACT | | | ACAGCGATTA | |
| | | | TTGAAGA | | | CTGGCAATGT | |
| | | | | | | CCCC | |
| Human | OT1 | GA0100_ | TCGTCGGCAGCGT | 712 | GA0100_ | GTCTCGTGGG | 715 |
| | | OT_B2+ | CAGATGTGTATA | | OT_B2− | CTCGGAGATG | |
| | | | AGAGACAGCCTG | | | TGTATAAGAG | |
| | | | GTCTCCTGTTCAC | | | ACAGAGAAAT | |
| | | | ACT | | | GGTTTGCTCA | |
| | | | | | | GAGGA | |
| Cyno- | On- | cAN_Ex6_ | TCGTCGGCAGCGT | 713 | cAN_Ex6_ | GTCTCGTGGG | 716 |
| mologus | target | 3_NEXT_ | CAGATGTGTATA | | 3_NEXT_ | CTCGGAGATG | |
| monkey | | F1 | AGAGACAGGGGA | | R1 | TGTATAAGAG | |
| | | | TTCGGATTTTTAA | | | ACAGCCCAAT | |
| | | | AAGTTGTC | | | GCAATCCCGG | |
| | | | | | | AAAA | |

After purification of the PCR amplicons with the ThermoFisher Sequalprep Normalization Plate Kit, a second round of PCR with Q5 HotStart polymerase was performed with Illumina primers containing unique dual indexes (UDIs), Nextera XT Index Kit V2 Set A and/or Nextera XT Index Kit V2 Set D, to generate barcoded libraries. Following a final purification with the Sequalprep Normalization Plate Kit, the individual amplicons were pooled and quantified using a Qubit 3.0 Fluorometer. After denaturation, dilution to 8 pM, and supplementation with 30% PhiX, the pooled libraries underwent 2×150-bp paired-end sequencing on an Illumina MiSeq System.

Targeted amplicon sequencing data were analyzed with CRISPResso2 v2.0.31 in batch mode (CRISPRessoBatch). For ABE experiments, the following parameters were set: “--default_min_aln_score 95--quantification_window_center-10--quantification_window_size 10--base_editor output --conversion_nuc_from A --conversion_nuc_to G --mn_frequency_alleles_around_cut to_plot 0.1--max_rows_alleles_around_cut to_plot 100”. For NHP experiments, an additional parameter was set to exclude low quality reads: “--min_single_bp_quality 30”. Moreover, in all cases, the parameter “--max_paired_end_reads_overlap” was set to 2R-F+0.25*F, following FLASH recommendations (http://ccb.jhu.edu/software/FLASH/), where R was the read length and F was the amplicon length.

For the on-target data from NHP samples, as well as for some of the cellular on-target data, editing was quantified from the “Quantification_window_nucleotide_percentage_table.txt” output table as the percentage of reads that supported any A-to-G/C/T substitution in the main edited position (position 6 of the protospacer DNA sequence). Indels were quantified from the “Alleles_frequency_table_around_sgRNA_*.txt” output table as the percentage of reads that supported insertions or deletions over a 5-bp window on either side of the nick site (at position −3 upstream of the PAM sequence), having excluded reads that supported deletions larger than 30 bp. The off-target site (OT1), as well as certain instances of assessing the on-target site, editing was quantified from the “Alleles_frequency_table_around_sgRNA_*.txt” output table as the percentage of reads from alleles with an A->G substitution in the editing window (positions 1-10 in the PAM distal side of the protospacer), having excluded reads from alleles with deletions larger than 30 bp. For each site, base editing was quantified by summing the percentage of reads supporting an A to G substitution in the editing window, requiring a minimum of 1000 reads.

### SureSelect Targeted Enrichment Sequencing.

SureSelect panels were designed and purchased from Agilent Technologies. Panels of SureSelect RNA probes for target enrichment were designed using the Agilent SureDesign module, using each candidate off-target site protospacer sequences nominated by ONE-seq (using GA441 and recombinant ABE8.8 protein), with an additional 150 nt genomic sequence flanking each side as the input. The RefSeq, Ensembl, CCDS, Gencode, VEGA, SNP, and CytoBand databases were used for the target parameters, with the least stringent masking and 3× tiling density applied in the design.

To perform SureSelect, 50-150 ng of genomic DNA, which contains approximately 60,000 copies of genome, was used for each experimental replicate. This ensures that over 1000 unique molecules were analyzed to support detection of rare substitution events. SureSelect assays were performed with the resulting RNA probes following Agilent's XT HS2 protocol. Briefly, gDNA samples were fragmented to 180 to 250 bp size range using a Covaris M220 focused ultrasonicator (75 W Peak Incident Power, 20% Duty Factor, 200 cycles/burst, for 150 seconds). Sheared gDNA samples were subjected to NGS library preparation steps: end-repair, dA-tailing, UMI adaptor ligation, whole genome amplification, probe hybridization and capturing, and post capturing amplification. The final library samples were sequenced externally by Broad Institute on an Illumina NovaSeq S4 with 2×150-bp paired-end sequencing. Two biological replicates for each condition, treated and control, were performed.

To analyze the SureSelect data, Adapters were trimmed using the “Trimmer” script in Agilent's AGeNT v2.0.5 tool. Reads were aligned to the GRCh38 reference human genome (ftp://ftp.ncbi.nlm.nih.gov/genomes/all/GCA/000/001/405/GCA_000001405.15_GRCh38/seqs_for_alignment_pipelines.ucsc_ids/GCA_000001405.15_GRCh38_no_alt_analysis_set.fna) using BWA MEM v 0.7.17-r1188 with parameter “—C”. Aligned reads were processed and duplicates were removed using the “LocatIt” script in Agilent's AGeNT v2.0.5 tool, with parameters “-i -R -IB -OB -U -L”. Nucleotide distributions at each position in the editing window (positions 1 to 10 in the PAM distal side of the protospacer) were determined using perbase v0.6.3 (https://github.com/sstadick/perbase), with parameters “base-depth -F 3848-Q 30”.

The first step of quantification of off-target editing was performed by computing allelic totals of reads containing A to G edits in the editing window of each site. This step was followed by statistical testing that tested the association of difference in allelic read counts to treatment condition (treated vs untreated label). For each site, base editing was quantified by summing the percentage of reads supporting an A-to-G substitution in the editing window, requiring a minimum of 1000 reads. Net editing (mean of treated edit % minus mean of untreated edit %) was computed as a measure of effect size of editing.

Editing across treated and control samples was then assessed for significance using P values derived from a logistic regression model that models the log odds ratio based on the proportion of edited reads, using the treatment vector treatmentᵢ (treated=1, control=0), as follows:

log πi1−πi=β0+β1*treatmenti log πi1−πi=β0+β1*treatmenti

The logistic regression model is fitted for each site and the significance calculation tests if the outcome (log odds) is associated with the treatment vector (i.e., the null hypothesis being β₁=0). Sites with P values of 0.05 or less and with editing levels in control samples less than 0.10% are shown as off-target sites.

### Lipid Nanoparticle Formulation and Analysis.

LNPs used were formulated as previously described (Conway, A. et al. 2019 Mol. Ther. 27, 866-877; Villiger, L. et al 2021 Nat. Biomed. Eng. 5, 179-189) and generated either by (1) microfluidic mixing using the Precision Nanosystems NanoAssemblr system according to the manufacturer's protocol, with some optimization for individual payloads or by (2) rapid inline mixing of a solution of lipid excipients in an organic solvent and an aqueous solution of gRNA and mRNA. The lipid solution generally comprises of a mixture of four formulation excipients namely: an amino lipid, a monomethoxypolyethylene glycol (or methoxypoluyethyle glycol) of average molecular weight 2000 Da conjugated to a lipid called PEG-Lipid, cholesterol and 1,2-distearoyl-sn-glycero-3-phosphocholine (DSPC), mixed in a predetermined molar ratio in ethanol. LNP composition comprised 40-65% of amino lipid, 2-20% DSPC, 1-5% PEG-Lipid, with the balance being cholesterol (all in mol %). The RNA aqueous solution contains a 1:1 by weight mixture of desired mRNA and guide RNA (gRNA) unless otherwise specified. For evaluating impact of mRNA to gRNA ratio, aqueous solution containing desired weight ratio of mRNA and gRNA were prepared prior to the preparation of corresponding LNPs, for evaluation; for example, the preparation of LNP test article to evaluate 6:1, 3:1, 2:1, 1:1, 1:2, 1:3 and 1:6 mRNA to gRNA ratio in mice. In some other instances, the mRNA and two gRNAs were mixed in 1:0.5:0.5 (mRNA:gRNA1:gRNA2) weight ratio to prepare LNPs containing desired mRNA and two gRNAs in a single test article. The aqueous solution of desired mRNA to gRNA was then mixed with the lipid excipients in ethanol by microfluidic or by rapid inline mixing. All LNPs for reported NHP studies were prepared by following the inline mixing protocols as described WO 2019/036028 A1, WO 2015/199952 A1, WO 2017/004143, WO 2020/081938 A1 and WO 2020/061426 A2. The PEG-Lipid used for the preparation of the NHP LNP test articles was selected from WO 2019/036028 A1, WO 2015/199952 A1. The LNP compositions and ionizable amino lipids were selected from the from Patent Publications WO/2017/004143A1, WO/2017/075531A1 and WO/2018/191719 A1.

As an example, the LNPs used for cellular and NHP studies had an average hydrodynamic diameter range of 55 to 70 nm, with a polydispersity index of <0.2 as determined by dynamic light scattering and 85-100% total RNA encapsulation as measured by the Quant-iT Ribogreen Assay. The LNP particle size (Z-Ave, hydrodynamic diameter), polydispersity index and total RNA encapsulation were measured as described in the literature prior to administration.

### Genomic DNA Extraction.

100200 mg of monkey liver was loaded into 2 mL lysing matrix tubes (MP Bio). Livers were lysed with 0.5 ml PBS for the mouse liver or 0.25 mL PBS for the monkey liver, using the FastPrep-24 system (MP Bio) according to the manufacturer's protocol. Genomic DNA was isolated from approximately 20 μL of monkey liver lysate using a bead-based extraction kit, MagMAX-96 DNA Multi-Sample Kit (Thermo-Fisher Scientific) on the KingFisher Flex automated extraction instrument (Thermo-Fisher Scientific) according to the manufacturer's protocols. For monkey liver biopsy samples, Qiagen DNEasy Blood & Tissue kit extraction was used to extract genomic DNA according to the manufacturer's instructions. Extracted genomic DNA was stored at 4° C. until further use or at −80° C. for long term storage.

### ANGPTL3 Protein Levels Quantified by ELISA.

The plasma cynomolgus ANGPTL3 levels were determined by ELISA; briefly, test samples or standards of purified cynomolgus monkey ANGPTL3 diluted in calibrator diluent RD6Q (R&D, part #895128) were incubated with assay diluent RD1-76 (R&D, part #895812) in a 96-well microplate coated with a monoclonal antibody specific for human ANGPTL3 (R&D, part #893734). After four washes with wash buffer (R&D, part #895003), human ANGPTL3 conjugate (R&D, part #893735) which contains a polyclonal antibody specific for human ANGPTL3 conjugated to horseradish peroxidase (HRP) were next incubated in individual wells. After four washes, TMB substrate solution (R&D, part #895000 and 895001) was used to develop the plate. Optical density was determined using on a microplate reader set to 450 nm. Readings at 540 nm were subtracted from the readings at 450 nm to correct for optical imperfections in the plate.

### Quantifying Lipid Levels.

Reagent kits each analyte contain reagent, cholesterol, triglycerides and HDL-C are quantified using absorbance measurements of specific enzymatic reaction products. LDL-C is determined indirectly. Most of the circulating cholesterol is found in three major lipoprotein fractions: very low-density lipoproteins (VLDL), LDL and HDL. [Total C]=[VLDL-C]+[LDL-C]+[HDL-C]. Thus the LDL-C can be calculated from measured values of total cholesterol, triglycerides and HDL-C according to the relationship: [LDL-C]=[total C]−[HDL-C]−[TG]/5, where [TG]/5 is an estimate of VLDL-cholesterol expressed.

### Direct Measurement of Triglyceride Levels.

A clinical analyzer instrument was used to measure a ‘lipid panel’ in serum samples. This entails the direct measurement of cholesterol (total C), triglycerides (TG) and high-density lipoprotein cholesterol (HDL-C). A reagent kit specific for triglycerides contains buffers, calibrators, blanks and controls. Using the provided reagents, serum samples from the study were analyzed. Triglycerides were measured using a series of coupled enzymatic reactions. H₂O₂ was the end product of the last one and its absorbance at 500 nm is used to quantify the analyte. The color intensity was proportional to triglyceride concentrations. All values were reported in mg/dL.

### LNP Treatment of NHPs.

The NHP studies were approved by the Institutional Animal Care and Use Committees of Envol Biomedical and Altasciences, respectively. NHPs studies were performed at Envol Biomedical (study #VTP2001) and Altasciences (study #1388.02, 04, 05, 09, and 11), with both studies using Macaca fascicularis) of Cambodian origin. The animals were 2-3 years of age and 2-3 kilograms in weight at the time of study initiation. All the animals were genotyped at the PCSK9 and/or ANGPTL3 editing site(s) to ensure that any animals receiving LNPs were homozygous for the protospacer DNA sequences perfectly matching the gRNA sequence, and animals were randomly assigned to various experimental groups. The animals were premedicated with 1 mg/kg dexamethasone, 0.5 mg/kg famotidine, and 5 mg/kg diphenhydramine prior to LNP administration unless otherwise stated. The LNP were administered using a temporary catheter inserted into a peripheral vein connected to a primed infusion line, over the course of 1 hour (+/−5 minutes). Dose formulations were administered at a volume of 6 ml/kg, unless otherwise specified, and dosed at mg/kg corresponding to mg of total RNA per animal weight (kilogram). The dose was calculated based on total RNA that constitute the amount of mRNA and gRNA, after formulating the LNP. The appropriate volume based on the weight of the animal was delivered using an infusion pump. Control animals received phosphate-buffered saline instead of LNP under the same infusion conditions. When there were two LNPs constituted from amino lipid 1 and amino lipid 2 used in the same NHP study, the test articles were identified as LNP #1 and LNP #2 where mRNA and gRNA used for preparing these LNPs were the same. If there was only one LNP composition used in a study, the test article was identified as LNP.

For blood chemistry samples, animals were fasted for at least 4 hours before collection via peripheral venipuncture. NHP studies generally followed collection on the following schedule: day −10, day −7, day −5, day 1 (6 hours after LNP infusion), day 2, day 3, day 5, day 8, and day 15. In the long-term study, samples were also collected at day 21 and day 28 and have generally been collected every 2 weeks thereafter and analyzed by the study site for LDL cholesterol, HDL cholesterol, total cholesterol, triglycerides, AST, and ALT. For each analyte, the mean of the values at day −10, day −7, and day −5 were regarded as the baseline value. A portion of each blood sample was sent to the investigators for ANGPTL3 protein measurement.

### Optical Genome Mapping.

A total of 750 ng high molecular weight (HMW) genomic DNA per sample was used for labeling according to Bionano Prep Direct Label and Stain DLS Protocol. Genomic DNA was labeled at a specific motif (CTTAAG) throughout the genome with DL-Green fluorophores by DLE-1 enzyme. After proteinase K digestion to inactivate the DLE-1 enzyme, samples were cleaned up through successive membrane adsorptions. Genomic DNA backbone was counterstained for size identification, incubated and homogenized overnight. The genomic DNA concentration of labeled samples was measured using the Qubit High Sensitivity dsDNA Assay Kit per manufacturer's instructions. The labeled samples with concentration of 4 to 12 ng/μL were loaded onto Saphyr chips and run on a Saphyr instrument to collect images of fluorescence-labeled genomic DNA molecules.

To identify treatment-specific structural variants (SVs), the Dual Analysis mode of the Structural Variant Annotation pipeline, following best practice recommendations from Bionano, was used for evaluating all SV calls from an untreated control and treated samples.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-219. (canceled)

220. A hybrid guide nucleic acid, wherein the hybrid guide nucleic acid comprises (i) a spacer sequence comprising a deoxyribonucleotide and a ribonucleotide comprising a ribose, wherein a 2′ hydroxyl group of the ribose is covalently linked to a methyl group (2′-OMe), and (ii) a binding scaffold.

221. The hybrid guide nucleic acid of claim 220, wherein the spacer sequence comprises an unmodified ribonucleotide.

222. The hybrid guide nucleic acid of claim 220, wherein the deoxyribonucleotide is located on position 3, 4, 6, 7 or 8 from the 5′ end of the spacer sequence.

223. The hybrid guide nucleic acid of claim 220, wherein the spacer sequence comprises deoxyribonucleotides on positions
3, 4, 6 and 7,
3 and 4,
6 and 7, or
3, 4, 6, 7 and 8,
from the 5′ end of the spacer sequence.

224. The hybrid guide nucleic acid of claim 220, wherein the spacer sequence comprises one to ten deoxyribonucleotides.

225. The hybrid guide nucleic acid of claim 220, wherein the spacer sequence comprises a phosphorothioate backbone modification (PS).

226. The hybrid guide nucleic acid of claim 220, wherein the spacer sequence comprises a (2′-OMe)PS(2′-OMe)PS(DNA)PS(DNA)(RNA)(DNA)(DNA) motif, optionally wherein the (2′-OMe)PS(2′-OMe)PS(DNA)PS(DNA)(RNA)(DNA)(DNA) motif is located at the 5′ end of the spacer sequence.

227. The hybrid guide nucleic acid of claim 2206, wherein the spacer sequence comprises a sequence with at least about 80%, about 90%, about 95%, about 99%, about 99.5%, or about 100% sequence identity or sequence similarity to SEQ ID NO: 28 or 29.

228. The hybrid guide nucleic acid of claim 220, wherein the spacer sequence comprises a (2′-OMe)PS(2′-OMe)PS(DNA)PS(DNA)(RNA)(DNA)(DNA)(DNA) motif, optionally wherein the (2′-OMe)PS(2′-OMe)PS(DNA)PS(DNA)(RNA)(DNA)(DNA)(DNA) motif is located at the 5′ end of the spacer sequence.

229. The hybrid guide nucleic acid of claim 220, wherein the spacer sequence comprises a sequence with at least about 80%, about 90%, about 95%, about 99%, about 99.5%, or about 100% sequence identity or sequence similarity to SEQ ID NO: 30 or 31.

230. The hybrid guide nucleic acid of claim 220, wherein the spacer sequence comprises a (2′-OMe)PS(2′-OMe)PS(DNA)PS(DNA)(DNA)(DNA)(DNA) motif, optionally wherein the (2′-OMe)PS(2′-OMe)PS(DNA)PS(DNA)(DNA)(DNA)(DNA) motif is located at the 5′ end of the spacer sequence.

231. The hybrid guide nucleic acid of claim 220, wherein the spacer sequence comprises a sequence with at least about 80%, about 90%, about 95%, about 99%, about 99.5%, or about 100% sequence identity or sequence similarity to SEQ ID NO: 11 or 12.

232. The hybrid guide nucleic acid of claim 220, wherein the spacer sequence corresponds to a protospacer on a target gene, and wherein the target gene is ANGPTL3.

233. The hybrid guide nucleic acid of claim 220, wherein the hybrid guide nucleic acid comprises a sequence with at least about 80%, about 90%, about 95%, about 99%, about 99.5%, or about 100% sequence identity or sequence similarity to a sequence selected from the group consisting of SEQ ID NOs: 79-82 and 106-113.

234. An in vivo hybrid guide gene editing system comprising:
(a) the hybrid guide nucleic acid of claim 220, and
(b) a gene editor protein or a component thereof comprising a nucleic acid binding domain, or a nucleic acid encoding the gene editor protein or the component thereof.

235. The in vivo hybrid guide gene editing system of claim 234, wherein the gene editor protein or the component thereof further comprises a deaminase.

236. The in vivo hybrid guide gene editing system of claim 234, wherein the nucleic acid binding domain is capable of binding to DNA or RNA.

237. The in vivo hybrid guide gene editing system of claim 234, wherein the nucleic acid binding domain comprises a CRISPR protein or a fragment thereof, a catalytically impaired nuclease, or a prime editing protein or a fragment thereof.

238. The in vivo hybrid guide gene editing system of claim 234, wherein the gene editor protein or the component thereof comprises a single fusion protein or two or more proteins.

239. The in vivo hybrid guide gene editing system of claim 234, wherein the nucleic acid encoding the gene editor protein or the component thereof is an mRNA.

240. The in vivo hybrid guide gene editing system of claim 234, wherein the gene editor protein or the component thereof affects less than about 10%, about 9%, about 8%, about 7%, about 6%, about 5%, about 4%, about 3%, about 2%, or about 1% editing on all off-target sites as compared to a gene editing system comprising a corresponding guide nucleic acid without the deoxyribonucleotide.

241. The in vivo hybrid guide gene editing system of claim 234, wherein the gene editor protein or the component thereof affects over about 50%, about 60%, about 70%, about 80%, about 90%, or about 95% editing on the target gene as compared to a gene editing system comprising a corresponding guide nucleic acid without the deoxyribonucleotide.

242. A pharmaceutical composition comprising the hybrid guide nucleic acid of claim 220 or the in vivo hybrid guide gene editing system of claim 234.

243. A method for treating or preventing an atherosclerotic cardiovascular disease in a subject in need thereof, the method comprising administering to the subject a therapeutically effective amount of the hybrid guide nucleic acid of claim 220, the in vivo hybrid guide gene editing system of claim 234, or the pharmaceutical composition of claim 242.
